(19) United States
(12) Patent Application Publication
(10) Pub. No.: US 2025/0346616 A1
(43) Pub. Date: Nov. 13, 2025

(54) ROCK INHIBITORS AND USES THEREOF

(71) Applicant: DIZAL (JIANGSU) PHARMACEUTICAL CO., LTD., Shanghai (CN)

(72) Inventors: Rumin WANG, Shanghai (CN); Changhe QI, Shanghai (CN); Honchung TSUI, Shanghai (CN); Qingbei ZENG, Shanghai (CN); Zhenfan YANG, Shanghai (CN); Xiaolin ZHANG, Shanghai (CN)

(21) Appl. No.: 18/869,379

(22) PCT Filed: May 23, 2023
(86) PCT No.: PCT/CN2023/095713

§ 371 Date: Nov. 26, 2024

(30) Foreign Application Priority Data
May 26, 2022 (WO) PCT/CN2022/095127

(51) Int. Cl. C07F 9/6558; A61K 31/4155; A61K 31/416; A61K 31/4178; A61K 31/437; A61K 31/4439; A61K 31/454; A61K 31/4725; A61K 31/496; A61K 31/519; A61K 31/675; A61K 45/06; C07D 401/14; C07D 403/04; C07D 403/14; C07D 405/14; C07D 471/04; C07D 487/04

(52) U.S. Cl. CPC C07F 9/65583; A61K 31/4155; A61K 31/416; A61K 31/4178; A61K 31/437; A61K 31/4439; A61K 31/454; A61K 31/4725; A61K 31/496; A61K 31/519; A61K 31/675; A61K 45/06; C07D 401/14; C07D 403/04; C07D 403/14; C07D 405/14; C07D 471/04; C07D 487/04

(57) ABSTRACT

Disclosed herein are compounds of Formula (I), and pharmaceutically acceptable salts thereof, that are useful as ROCK inhibitors. Also disclosed are pharmaceutical compositions comprising a compound of Formula (I), and methods of using such compounds or compositions to treat ROCK-related disorder (e.g., glaucoma).

### FIELD OF THE DISCLOSURE

The present disclosure generally relates to novel compounds inhibiting Rho-related protein kinase (“ROCK”), and pharmaceutically acceptable salts thereof. The present disclosure also relates to pharmaceutical compositions comprising the compound as an active ingredient and use of the compounds in the treatment of ROCK-related disorder, including glaucoma.

### BACKGROUND

Rho-related protein kinase (ROCK) is a member of the serine-threonine protein kinase family. ROCK exists in two isoforms, ROCK1 and ROCK2. Both isoforms are activated by GTP-bound forms of Rho GTPase. ROCK plays important roles in numerous cellular processes including smooth muscle cell contraction, cell proliferation, adhesion and migration. Inhibition of ROCK activity has demonstrated potential therapeutic applicability in a wide range of pathological conditions.

Currently available ROCK inhibitor drugs include Eril (for the treatment of cerebral vasospasm) from Asahi Kasei, Glanatec (for the treatment of ocular hypertension and glaucoma) from Kowa, and Rhopressa (for the reduction of elevated intraocular pressure (IOP) in patients with open-angle glaucoma or ocular hypertension) from Aerie.

Therefore, novel compounds that inhibiting ROCK are needed as pharmacological tools and are of considerable interest as drugs for treating ROCK related disorders such as glaucoma.

### SUMMARY OF THE DISCLOSURE

Disclosed herein are novel compounds that are capable of inhibiting ROCK. As a result, the compounds of the present disclosure are useful in the treatment of ROCK-related diseases such as glaucoma.

In one aspect, the present disclosure provides a compound of Formula (I):

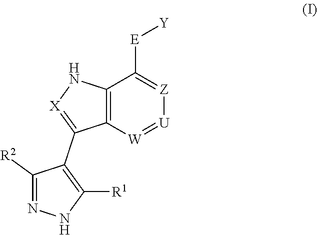

or a pharmaceutically acceptable salt thereof,
wherein,
   each of R¹ and R² is independently selected from the group consisting of hydrogen, hydroxy, halogen, cyano, amino, alkyl, alkenyl, alkynyl, heteroalkyl, heteroalkenyl, and heteroalkynyl, wherein the alkyl, alkenyl, alkynyl, heteroalkyl, heteroalkenyl, and heteroalkynyl are each optionally substituted with one or more groups independently selected from hydroxyl, halogen, cyano or amino;
   each of X, W, Z and U is independently N or C(R³);
      R³ is selected from the group consisting of hydrogen, hydroxy, halogen, cyano, amino, alkyl, alkenyl, alkynyl, heteroalkyl, heteroalkenyl, and heteroalkynyl, wherein the alkyl, alkenyl, alkynyl, heteroalkyl, heteroalkenyl, and heteroalkynyl are each optionally substituted with one or more groups independently selected from hydroxyl, halogen, cyano or amino;
   E is —N(R⁴)C(═O)—, —C(═O)N(R⁴)—, —N(R⁴)SO₂—, or —SO₂N(R⁴)—;
      R⁴ is selected from the group consisting of hydrogen, alkyl, alkenyl, alkynyl, heteroalkyl, heteroalkenyl, and heteroalkynyl;
   Y is —Y¹—Y²—(Y³)ₙ, wherein
      Y¹ is null or —C(R⁵)₂—,
         each R⁵ is independently selected from the group consisting of hydrogen, —N(R^{a})₂, alkyl, alkenyl, alkynyl, heteroalkyl, heteroalkenyl, heteroalkynyl, cycloalkyl, heterocyclyl, aryl, heteroaryl, and -alkyl-heterocyclyl, wherein the alkyl, alkenyl, alkynyl, heteroalkyl, heteroalkenyl, heteroalkynyl, cycloalkyl, heterocyclyl, aryl, heteroaryl and -alkyl-heterocyclyl are optionally substituted with one or more R⁶,
         each R⁶ is independently selected from the group consisting of hydroxy, halogen, cyano, amino, alkyl, alkenyl, alkynyl, heteroalkyl, heteroalkenyl, heteroalkynyl, and -alkyl-N(R^{a})₂;
         each R^{a} is independently selected from hydrogen or alkyl;
      Y² is selected from —(CH₂)ₚ-cycloalkyl-*, —(CH₂)ₚ-heterocyclyl-*, —(CH₂)ₚ-aryl-*, or —(CH₂)ₚ-heteroaryl-*, each of which can be optionally substituted with one or more groups independently selected from hydroxy, halogen, cyano, amino, alkyl, alkenyl, alkynyl, heteroalkyl, heteroalkenyl, or heteroalkynyl, and wherein p is 0, 1 or 2, and * end of Y² is connected to Y³;
      Y³ is null or —Y³¹—Y³²—Y³³; wherein
         Y³¹ is selected from null, alkyl, alkenyl or alkynyl;
         Y³² is selected from null, —O—#, —OC(═O)—#, —C(═O)O—#, —P(═O)(R^{b})—#, —OC(═O)N(R^{b})—#, —N(R^{b})C(═O)—#, or —C(═O)N(R^{b})—#, wherein each R^{b} is independently selected from hydrogen or alkyl, and #end of Y³² is connected to Y³³;
         Y³³ is selected from a group consisting of hydrogen, hydroxyl, cyano, halogen, —N(R^{c})₂, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl, and heteroaryl are optionally substituted with one or more R⁷, wherein each R⁷ is independently selected from halogen, hydroxy, amino, cyano, nitrooxy, alkyl, alkenyl, alkynyl, heteroalkyl, heteralkenyl, heteroalkynyl, or haloalkyl, and each R^{c} is independently selected from hydrogen or alkyl; and n is an integer from 1 to 5.

In another aspect, the present disclosure provides a pharmaceutical composition comprising the compound of the present disclosure or a pharmaceutically acceptable salt thereof, and a pharmaceutically acceptable carrier.

In a further aspect, the present disclosure provides a method for inhibiting ROCK activity in a subject in need thereof, comprising administering an effective amount of a compound of the present disclosure or a pharmaceutically acceptable salt thereof or the pharmaceutical composition of the present disclosure to the subject.

In a further aspect, the present disclosure provides a method for treating a ROCK related disorder comprising administering an effective amount of a compound of the present disclosure or a pharmaceutically acceptable salt thereof or the pharmaceutical composition of the present disclosure to a subject in need thereof.

In another aspect, the present disclosure provides use of the compound of the present disclosure or a pharmaceutically acceptable salt thereof or the pharmaceutical composition of the present disclosure in the manufacture of a medicament for treating ROCK-related disorders.

In another aspect, the present disclosure provides a compound of present disclosure or a pharmaceutically acceptable salt thereof or the pharmaceutical composition of the present disclosure, for use in the treatment of ROCK-related disorder.

### DETAILED DESCRIPTION OF THE DISCLOSURE

Reference will now be made in detail to certain embodiments of the present disclosure, examples of which are illustrated in the accompanying structures and formulas. While the present disclosure will be described in conjunction with the enumerated embodiments, it will be understood that they are not intended to limit the present disclosure to those embodiments. On the contrary, the present disclosure is intended to cover all alternatives, modifications, and equivalents, which may be included within the scope of the present disclosure as defined by the claims. One skilled in the art will recognize many methods and materials similar or equivalent to those described herein, which could be used in the practice of the present disclosure. The present disclosure is in no way limited to the methods and materials described. In the event that one or more of the incorporated references and similar materials differs from or contradicts this application, including but not limited to defined terms, term usage, described techniques, or the like, the present disclosure controls. All references, patents, patent applications cited in the present disclosure are hereby incorporated by reference in their entireties.

It is appreciated that certain features of the present disclosure, which are, for clarity, described in the context of separate embodiments, can also be provided in combination in a single embodiment. Conversely, various features of the present disclosure, which are, for brevity, described in the context of a single embodiment, can also be provided separately or in any suitable sub-combination. It must be noted that, as used in the specification and the appended claims, the singular forms “a,” “an,” and “the” include plural forms of the same unless the context clearly dictates otherwise. Thus, for example, reference to “a compound” includes a plurality of compounds.

### Definition

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

At various places in the present disclosure, linking substituents are described. Where the structure clearly requires a linking group, the Markush variables listed for that group are understood to be linking groups. For example, if the structure requires a linking group and the Markush group definition for that variable lists “alkyl”, then it is understood that the “alkyl” represents a linking alkylene group.

When any variable (e.g., Rⁱ) occurs more than one time in any constituent or formula for a compound, its definition at each occurrence is independent of its definition at every other occurrence. Thus, for example, if a group is shown to be substituted with 0-2 Rⁱ moieties, then the group may optionally be substituted with up to two Rⁱ moieties and Rⁱ at each occurrence is selected independently from the definition of Rⁱ. Also, combinations of substituents and/or variables are permissible, but only if such combinations result in stable compounds.

As used herein, a dash “—” at the front or end of a chemical group is used, a matter of convenience, to indicate a point of attachment for a substituent. For example, —OH is attached through the oxygen atom; chemical groups may be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named. As used herein, a solid line coming out of the center of a ring indicates that the point of attachment for a substituent on the ring can be at any ring atom. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

Recitation of ranges of values are merely intended to serve as a shorthand method of referring individually to each separate value falling within the range, unless otherwise indicated herein, and each separate value is incorporated into the specification as if it were individually recited herein. A range used herein, unless otherwise specified, includes the two limits of the range. For example, the expressions “n is an integer between 1 and 6” and “n being an integer of 1 to 6” both mean “n being 1, 2, 3, 4, 5, or 6”.

As used herein, the term “compounds provided herein”, or “compounds disclosed herein” or “compounds of the present disclosure” refers to the compounds of Formula (I) as well as the specific compounds disclosed herein.

As used herein, the term “Cᵢ₋ⱼ” indicates a range of the carbon atoms numbers, wherein i and j are integers and the range of the carbon atoms numbers includes the endpoints (i.e. i and j) and each integer point in between, and wherein j is greater than i. For examples, C₁₋₆ indicates a range of one to six carbon atoms, including one carbon atom, two carbon atoms, three carbon atoms, four carbon atoms, five carbon atoms and six carbon atoms. In some embodiments, the term “C₁₋₁₂” indicates 1 to 12, particularly 1 to 10, particularly 1 to 8, particularly 1 to 6, particularly 1 to 5, particularly 1 to 4, particularly 1 to 3 or particularly 1 to 2 carbon atoms.

As used herein, the term “alkyl”, whether as part of another term or used independently, refers to a saturated linear or branched-chain hydrocarbon radical, which may be optionally substituted independently with one or more substituents described below. The term “Cᵢ₋ⱼ alkyl” refers to an alkyl having i to j carbon atoms. In some embodiments, alkyl groups contain 1 to 10 carbon atoms. In some embodiments, alkyl groups contain 1 to 9 carbon atoms. In some embodiments, alkyl groups contain 1 to 8 carbon atoms, 1 to 7 carbon atoms, 1 to 6 carbon atoms, 1 to 5 carbon atoms, 1 to 4 carbon atoms, 1 to 3 carbon atoms, or 1 to 2 carbon atoms. Examples of “C₁₋₁₀ alkyl” include, but are not limited to, methyl, ethyl, propyl, butyl, pentyl, hexyl, heptyl, octyl, nonyl, and decyl. Examples of “C₁₋₆ alkyl” are methyl, ethyl, propyl, isopropyl, n-butyl, i-butyl, s-butyl, t-butyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, and the like.

As used herein, the term “alkenyl”, whether as part of another term or used independently, refers to linear or branched-chain hydrocarbon radical having at least one carbon-carbon double bond, which may be optionally substituted independently with one or more substituents described herein, and includes radicals having “cis” and “trans” orientations, or alternatively, “E” and “Z” orientations. In some embodiments, alkenyl groups contain 2 to 12 carbon atoms. In some embodiments, alkenyl groups contain 2 to 11 carbon atoms. In some embodiments, alkenyl groups contain 2 to 11 carbon atoms, 2 to 10 carbon atoms, 2 to 9 carbon atoms, 2 to 8 carbon atoms, 2 to 7 carbon atoms, 2 to 6 carbon atoms, 2 to 5 carbon atoms, 2 to 4 carbon atoms, 2 to 3 carbon atoms, and in some embodiments, alkenyl groups contain 2 carbon atoms. Examples of alkenyl group include, but are not limited to, ethylenyl (or vinyl), propenyl (allyl), butenyl, pentenyl, 1-methyl-2 buten-1-yl, 5-hexenyl, and the like.

As used herein, the term “alkynyl”, whether as part of another term or used independently, refers to a linear or branched hydrocarbon radical having at least one carbon-carbon triple bond, which may be optionally substituted independently with one or more substituents described herein. In some embodiments, alkenyl groups contain 2 to 12 carbon atoms. In some embodiments, alkynyl groups contain 2 to 11 carbon atoms. In some embodiments, alkynyl groups contain 2 to 11 carbon atoms, 2 to 10 carbon atoms, 2 to 9 carbon atoms, 2 to 8 carbon atoms, 2 to 7 carbon atoms, 2 to 6 carbon atoms, 2 to 5 carbon atoms, 2 to 4 carbon atoms, 2 to 3 carbon atoms, and in some embodiments, alkynyl groups contain 2 carbon atoms. Examples of alkynyl group include, but are not limited to, ethynyl, 1-propynyl, 2-propynyl, and the like.

As used herein, the term “amino” refers to —NH₂ group. Amino groups may also be substituted with one or more groups such as alkyl, aryl, carbonyl or other amino groups.

As used herein, the term “aryl”, whether as part of another term or used independently, refers to monocyclic and polycyclic ring systems having a total of 5 to 20 ring members, wherein at least one ring in the system is aromatic and wherein each ring in the system contains 3 to 12 ring members. Examples of “aryl” include, but are not limited to, phenyl, biphenyl, naphthyl, anthracyl and the like, which may bear one or more substituents. Also included within the scope of the term “aryl”, as it is used herein, is a group in which an aromatic ring is fused to one or more additional rings. In the case of polycyclic ring system, only one of the rings needs to be aromatic (e.g., 2,3-dihydroindole), although all of the rings may be aromatic (e.g., quinoline). The second ring can also be fused or bridged. Examples of polycyclic aryl include, but are not limited to, benzofuranyl, indanyl, phthalimidyl, naphthimidyl, phenanthridinyl, or tetrahydronaphthyl, and the like. Aryl groups can be substituted at one or more ring positions with substituents as described above.

As used herein, the term “cyano” refers to —CN.

As used herein, the term “cycloalkyl”, whether as part of another term or used independently, refer to a monovalent non-aromatic, saturated or partially unsaturated monocyclic and polycyclic ring system, in which all the ring atoms are carbon and which contains at least three ring forming carbon atoms. In some embodiments, the cycloalkyl may contain 3 to 12 ring forming carbon atoms, 3 to 10 ring forming carbon atoms, 3 to 9 ring forming carbon atoms, 3 to 8 ring forming carbon atoms, 3 to 7 ring forming carbon atoms, 3 to 6 ring forming carbon atoms, 3 to 5 ring forming carbon atoms, 4 to 12 ring forming carbon atoms, 4 to 10 ring forming carbon atoms, 4 to 9 ring forming carbon atoms, 4 to 8 ring forming carbon atoms, 4 to 7 ring forming carbon atoms, 4 to 6 ring forming carbon atoms, 4 to 5 ring forming carbon atoms. Cycloalkyl groups may be saturated or partially unsaturated. Cycloalkyl groups may be substituted. In some embodiments, the cycloalkyl group may be a saturated cyclic alkyl group. In some embodiments, the cycloalkyl group may be a partially unsaturated cyclic alkyl group that contains at least one double bond or triple bond in its ring system. In some embodiments, the cycloalkyl group may be monocyclic or polycyclic. The fused, spiro and bridged ring systems are also included within the scope of this definition. Examples of monocyclic cycloalkyl group include, but are not limited to, cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl and cyclododecyl. Examples of polycyclic cycloalkyl group include, but are not limited to, adamantyl, norbornyl, fluorenyl, spiro-pentadienyl, spiro[3.6]-decanyl, bicyclo[1,1,1]pentenyl, bicyclo[2,2,1]heptenyl, and the like.

As used herein, the term “halogen” refers to an atom selected from fluorine (or fluoro), chlorine (or chloro), bromine (or bromo) and iodine (or iodo).

As used herein, the term “heteroatom” refers to nitrogen, oxygen, or sulfur, and includes any oxidized form of nitrogen or sulfur, and any quaternized form of a basic nitrogen (including N-oxides).

As used herein, the term “heteroalkyl” refers to an alkyl, at least one of the carbon atoms of which is replaced with a heteroatom selected from N, O, or S. The heteroalkyl may be a carbon radical or heteroatom radical (i.e., the heteroatom may appear in the middle or at the end of the radical), and may be optionally substituted independently with one or more substituents described herein. The term “heteroalkyl” encompasses alkoxy and heteroalkoxy radicals.

As used herein, the term “heteroalkenyl” refers to an alkenyl, at least one of the carbon atoms of which is replaced with a heteroatom selected from N, O, or S. The heteroalkenyl may be a carbon radical or heteroatom radical (i.e., the heteroatom may appear in the middle or at the end of the radical), and may be optionally substituted independently with one or more substituents described herein.

As used herein, the term “heteroalkynyl” refers to an alkynyl, at least one of the carbon atoms of which is replaced with a heteroatom selected from N, O, or S. The heteroalkynyl may be a carbon radical or heteroatom radical (i.e., the heteroatom may appear in the middle or at the end of the radical), and may be optionally substituted independently with one or more substituents described herein.

As used herein, the term “heteroaryl”, whether as part of another term or used independently, refers to an aryl group having, in addition to carbon atoms, one or more heteroatoms. The heteroaryl group can be monocyclic. Examples of monocyclic heteroaryl include, but are not limited to, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, benzofuranyl and pteridinyl. The heteroaryl group also includes polycyclic groups in which a heteroaromatic ring is fused to one or more aryl, cycloaliphatic, or heterocyclyl rings, where the radical or point of attachment is on the heteroaromatic ring. Examples of polycyclic heteroaryl include, but are not limited to, indolyl, isoindolyl, benzothienyl, benzofuranyl, benzo[1,3]dioxolyl, dibenzofuranyl, indazolyl, benzimidazolyl, benzthiazolyl, quinolyl, isoquinolyl, dihydroquinolinyl, dihydroisoquinolinyl, tetrahydroquinolinyl, tetrahydroisoquinolinyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and the like.

As used herein, the term “heterocyclyl” refers to a saturated or partially unsaturated carbocyclyl group in which one or more ring atoms are heteroatoms independently selected from oxygen, sulfur, nitrogen, phosphorus, and the like, the remaining ring atoms being carbon, wherein one or more ring atoms may be optionally substituted independently with one or more substituents. In some embodiments, the heterocyclyl is a saturated heterocyclyl. In some embodiments, the heterocyclyl is a partially unsaturated heterocyclyl having one or more double bonds in its ring system. In some embodiments, the heterocyclyl may contains any oxidized form of carbon, nitrogen or sulfur, and any quaternized form of a basic nitrogen. “Heterocyclyl” also includes radicals wherein the heterocyclyl radicals are fused with a saturated, partially unsaturated, or fully unsaturated (i.e., aromatic) carbocyclic or heterocyclic ring. The heterocyclyl radical may be carbon linked or nitrogen linked where such is possible. In some embodiments, the heterocycle is carbon linked. In some embodiments, the heterocycle is nitrogen linked. For example, a group derived from pyrrole may be pyrrol-1-yl (nitrogen linked) or pyrrol-3-yl (carbon linked). Further, a group derived from imidazole may be imidazol-1-yl (nitrogen linked) or imidazol-3-yl (carbon linked).

In some embodiments, the term “3- to 12-membered heterocyclyl” refers to a 3- to 12-membered saturated or partially unsaturated monocyclic or polycyclic heterocyclic ring system having 1 to 3 heteroatoms independently selected from nitrogen, oxygen, or sulfur. The fused, spiro and bridged ring systems are also included within the scope of this definition. Examples of monocyclic heterocyclyl include, but are not limited to oxetanyl, 1,1-dioxothietanylpyrrolidyl, tetrahydrofuryl, tetrahydrothienyl, pyrrolyl, furanyl, thienyl, pyrazolyl, imidazolyl, triazolyl, oxazolyl, thiazolyl, piperidyl, piperazinyl, piperidinyl, morpholinyl, pyridinyl, pyrazinyl, pyrimidinyl, pyridazinyl, triazinyl, pyridonyl, pyrimidonyl, pyrazinonyl, pyrimidonyl, pyridazonyl, pyrrolidinyl, triazinonyl, and the like. Examples of fused heterocyclyl include, but are not limited to, phenyl fused ring or pyridinyl fused ring, such as quinolinyl, isoquinolinyl, tetrahydroquinolinyl, tetrahydroisoquinolinyl, quinoxalinyl, quinolizinyl, quinazolinyl, azaindolizinyl, pteridinyl, chromenyl, isochromenyl, indolyl, isoindolyl, indolizinyl, indazolyl, purinyl, benzofuranyl, isobenzofuranyl, benzimidazolyl, benzothienyl, benzothiazolyl, carbazolyl, phenazinyl, phenothiazinyl, phenanthridinyl, hexahydro-1H-pyrrolizinyl, imidazo[1,2-a]pyridinyl, [1,2,4]triazolo[4,3-a]pyridinyl, [1,2,3]triazolo[4,3-a]pyridinyl groups, and the like. Examples of spiro heterocyclyl include, but are not limited to, spiropyranyl, spirooxazinyl, and the like. Examples of bridged heterocyclyl include, but are not limited to, morphanyl, hexamethylenetetraminyl, 3-aza-bicyclo[3.1.0]hexane, 8-aza-bicyclo[3.2.1]octane, 1-aza-bicyclo[2.2.2]octane, 1,4-diazabicyclo[2.2.2]octane (DABCO), and the like.

As used herein, the term “hydroxyl” or “hydroxy” refers to —OH.

As used herein, the term “partially unsaturated” refers to a radical that includes at least one double or triple bond. The term “partially unsaturated” is intended to encompass rings having multiple sites of unsaturation, but is not intended to include aromatic (i.e., fully unsaturated) moieties.

As used herein, the term “optional” or “optionally” means that the subsequently described event or circumstance may or may not occur, and that the description includes instances where the said event or circumstance occurs and instances in which it does not. As used herein, the term “substituted”, whether preceded by the term “optionally” or not, means that one or more hydrogens of the designated moiety are replaced with a suitable substituent. It will be understood that “substitution” or “substituted with” includes the implicit proviso that such substitution is in accordance with permitted valence of the substituted atom and that the substitution results in a stable or chemically feasible compound, e.g., which does not spontaneously undergo transformation such as by rearrangement, cyclization, elimination, etc. Unless otherwise indicated, an “optionally substituted” group may have a suitable substituent at each substitutable position of the group, and when more than one position in any given structure may be substituted with more than one substituent selected from a specified group, the substituent may be either the same or different at every position. The substituents may include, but not limited to, alkyl, alkenyl, alkynyl, alkoxy, acyl, amino, amido, amidino, aryl, azido, carbamoyl, carboxyl, carboxyl ester, cyano, guanidino, halo, haloalkyl, heteroalkyl, heteroaryl, heterocyclyl, hydroxy, hydrazino, imino, oxo, nitro, alkylsulfinyl, sulfonic acid, alkylsulfonyl, thiocyanate, thiol, thione, or combinations thereof. It will be understood by those skilled in the art that substituents can themselves be substituted, if appropriate. Unless specifically stated as “unsubstituted”, references to chemical moieties herein are understood to include substituted variants. For example, reference to an “aryl” group or moiety implicitly includes both substituted and unsubstituted variants.

As used herein, the term “substituted”, whether preceded by the term “optionally” or not, means that one or more hydrogens of the designated moiety are replaced with a suitable substituent. It will be understood that “substitution” or “substituted with” includes the implicit proviso that such substitution is in accordance with permitted valence of the substituted atom and that the substitution results in a stable or chemically feasible compound, e.g., which does not spontaneously undergo transformation such as by rearrangement, cyclization, elimination, etc. Unless otherwise indicated, an “optionally substituted” group may have a suitable substituent at each substitutable position of the group, and when more than one position in any given structure may be substituted with more than one substituent selected from a specified group, the substituent may be either the same or different at every position. It will be understood by those skilled in the art that substituents can themselves be substituted, if appropriate. Unless specifically stated as “unsubstituted”, references to chemical moieties herein are understood to include substituted variants. For example, reference to an “aryl” group or moiety implicitly includes both substituted and unsubstituted variants.

The symbols “R” and “S” represent the configuration of substituents around a chiral carbon atom(s). The isomeric descriptors “R” and “S” are used as described herein for indicating atom configuration(s) relative to a core molecule and are intended to be used as defined in the literature (IUPAC Recommendations 1996, Pure and Applied Chemistry, 68:2193-2222 (1996)).

### Compound

In one aspect, the present disclosure provides compounds of Formula (I):

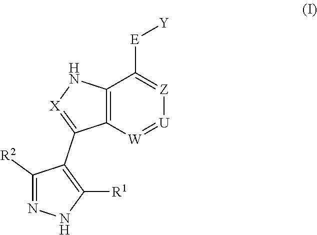

or a pharmaceutically acceptable salt thereof,
wherein,
   each of R¹ and R² is independently selected from the group consisting of hydrogen, hydroxy, halogen, cyano, amino, alkyl, alkenyl, alkynyl, heteroalkyl, heteroalkenyl, and heteroalkynyl, wherein the alkyl, alkenyl, alkynyl, heteroalkyl, heteroalkenyl, and heteroalkynyl are each optionally substituted with one or more groups independently selected from hydroxyl, halogen, cyano or amino;
   each of X, W, Z and U is independently N or C(R³);
      R³ is selected from the group consisting of hydrogen, hydroxy, halogen, cyano, amino, alkyl, alkenyl, alkynyl, heteroalkyl, heteroalkenyl, and heteroalkynyl, wherein the alkyl, alkenyl, alkynyl, heteroalkyl, heteroalkenyl, and heteroalkynyl are each optionally substituted with one or more groups independently selected from hydroxyl, halogen, cyano or amino;
   E is —N(R⁴)C(═O)—, —C(═O)N(R⁴)—, —N(R⁴)SO₂—, or —SO₂N(R⁴)—;
      R⁴ is selected from the group consisting of hydrogen, alkyl, alkenyl, alkynyl, heteroalkyl, heteroalkenyl, and heteroalkynyl;
   Y is —Y¹—Y²—(Y³)ₙ, wherein
      Y¹ is null or —C(R⁵)₂—,
         each R⁵ is independently selected from the group consisting of hydrogen, —N(R^{a})₂, alkyl, alkenyl, alkynyl, heteroalkyl, heteroalkenyl, heteroalkynyl, cycloalkyl, heterocyclyl, aryl, heteroaryl, and -alkyl-heterocyclyl, wherein the alkyl, alkenyl, alkynyl, heteroalkyl, heteroalkenyl, heteroalkynyl, cycloalkyl, heterocyclyl, aryl, heteroaryl and -alkyl-heterocyclyl are optionally substituted with one or more R⁶,
         each R⁶ is independently selected from the group consisting of hydroxy, halogen, cyano, amino, alkyl, alkenyl, alkynyl, heteroalkyl, heteroalkenyl, heteroalkynyl, and -alkyl-N(R^{a})₂;
         each R^{a} is independently selected from hydrogen or alkyl;
      Y² is selected from —(CH₂)ₚ-cycloalkyl-*, —(CH₂)ₚ-heterocyclyl-*, —(CH₂)ₚ-aryl-*, or —(CH₂)ₚ-heteroaryl-*, each of which can be optionally substituted with one or more groups independently selected from hydroxy, halogen, cyano, amino, alkyl, alkenyl, alkynyl, heteroalkyl, heteroalkenyl, or heteroalkynyl, and wherein p is 0, 1 or 2, and * end of Y² is connected to Y³;
      Y³ is null or —Y³¹—Y³²—Y³³; wherein
         Y³¹ is selected from null, alkyl, alkenyl or alkynyl;
         Y³² is selected from null, —O—#, —OC(═O)—#, —C(═O)O—#, —P(═O)(R^{b})—#, —OC(═O)N(R^{b})—#, —N(R^{b})C(═O)—#, or —C(═O)N(R^{b})—#, wherein each R^{b} is independently selected from hydrogen or alkyl, and #end of Y³² is connected to Y³³;
         Y³³ is selected from a group consisting of hydrogen, hydroxyl, cyano, halogen, —N(R^{c})₂, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl, and heteroaryl are optionally substituted with one or more R⁷, wherein each R⁷ is independently selected from halogen, hydroxy, amino, cyano, nitrooxy, alkyl, alkenyl, alkynyl, heteroalkyl, heteralkenyl, heteroalkynyl, or haloalkyl, and each R^{e} is independently selected from hydrogen or alkyl; and
   n is an integer from 1 to 5.

In some embodiments, each of R¹ and R² is independently hydrogen, halogen, hydrogen, cyano, amino, or alkyl optionally substituted with one or more halogens.

In some embodiments, R¹ is hydrogen or alkyl, and R² is hydrogen, halogen or alkyl. In certain embodiments, R¹ is hydrogen or C₁₋₆ alkyl, and R² is hydrogen, halogen or C₁₋₆ alkyl.

In some embodiments, R¹ and R² are both hydrogen.

In some embodiments, R¹ and R² are both alkyl. In certain embodiments, R¹ is C₁₋₆ alkyl, C₁₋₅ alkyl, C₁₋₄ alkyl, C₁₋₃ alkyl or C₁₋₂ alkyl, and R² is C₁₋₆ alkyl, C₁₋₅ alkyl, C₁₋₄ alkyl, C₁₋₃ alkyl or C₁₋₂ alkyl. In certain embodiments, R¹ is methyl, and R² is methyl.

In some embodiments, R¹ is hydrogen, and R² is halogen. In certain embodiments, R¹ is hydrogen, and R² is fluoro.

In some embodiments, R¹ is hydrogen, and R² is alkyl. In certain embodiments, R¹ is hydrogen, and R² is C₁₋₆ alkyl, C₁₋₅ alkyl, C₁₋₄ alkyl, C₁₋₃ alkyl or C₁₋₂ alkyl. In certain embodiments, R¹ is hydrogen, and R² is methyl.

In some embodiments, X, W, U and Z are C(R³). In certain embodiments, W, U and Z are C(R³), and R³ is hydrogen.

In some embodiments, X and U are C(R³), and W and Z are N. In certain embodiments, X and U are C(R³) where R³ is hydrogen, and W and Z are N.

In some embodiments, E is —N(R⁴)C(═O)— or —C(═O)N(R⁴)—. In certain embodiments, E is —N(R⁴)C(═O)— or —C(═O)N(R⁴)—, and R⁴ is hydrogen.

In some embodiments, Y¹ is null.

In some embodiments, Y¹ is —C(R⁵)₂—.

In certain embodiments, Y¹ is —C(R⁵)₂—, one R⁵ is hydrogen, the other R⁵ is selected from —N(R^{a})₂, alkyl, cycloalkyl, heterocyclyl and -alkyl-heterocyclyl, wherein the alkyl, cycloalkyl, heterocyclyl and -alkyl-heterocyclyl are optionally substituted with one or more R⁶.

In certain embodiments, Y¹ is —C(R⁵)₂—, one R⁵ is hydrogen, the other R⁵ is selected from —N(R^{a})₂, C₁₋₆ alkyl, C₃-12 cycloalkyl, 5- to 12-membered heterocyclyl or —(C₁₋₆ alkyl)-(5- to 12-membered heterocyclyl), wherein the C₁₋₆ alkyl, C₃-12 cycloalkyl, 5- to 12-membered heterocyclyl and —(C₁₋₆ alkyl)-(5- to 12-membered heterocyclyl) are optionally substituted with one or more R⁶.

In certain embodiments, Y¹ is —C(R⁵)₂—, one R⁵ is alkyl, the other R⁵ is selected from —N(R^{a})₂, alkyl, cycloalkyl, or heterocyclyl, wherein the alkyl, cycloalkyl, and heterocyclyl are optionally substituted with one or more R⁶.

In certain embodiments, Y¹ is —C(R⁵)₂—, one R⁵ is C₁₋₆ alkyl, the other R⁵ is selected from —N(R^{a})₂, C₁₋₆ alkyl, C₃-12 cycloalkyl, or 5- to 12-membered heterocyclyl, wherein the C₁-6 alkyl, C₃₋₁₂ cycloalkyl, or 5- to 12-membered heterocyclyl are optionally substituted with one or more R⁶.

In some embodiments, R⁶ is selected from amino, alkyl or -alkyl-N(R^{a})₂. In certain embodiments, R⁶ is selected from amino, C₁₋₆ alkyl or —(C₁₋₆ alkyl)-N(R^{a})₂.

In some embodiments, Y¹ is selected from the group consisting of:

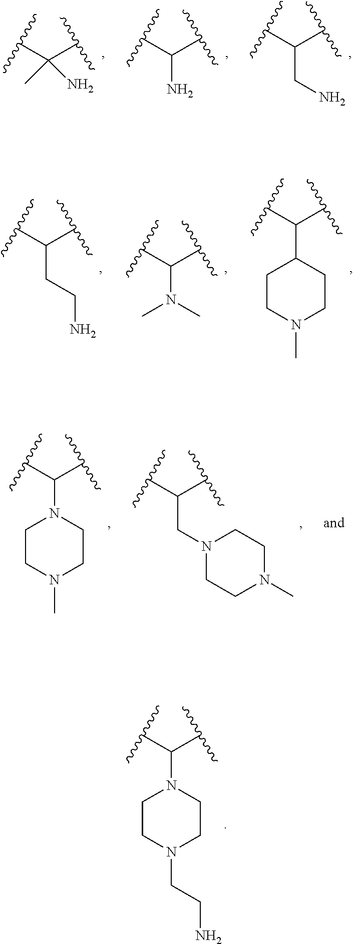

In some embodiments, Y² is —(CH₂)ₚ-cycloalkyl-*.

In certain embodiments, Y² is —(CH₂)ₚ—(C₃₋₁₀ cycloalkyl)-*, and p is 0 or 1 In certain embodiments, Y² is cyclopentyl or cyclohexyl.

In certain embodiments, Y² is —(CH₂)ₚ-heterocyclyl-*.

In certain embodiments, Y² is —(CH₂)ₚ-(5- to 12-membered heterocyclyl)-*, and p is 0 or 1.

In certain embodiments, Y² is tetrahydrofuranyl, pyrrolidinyl, or tetrahydropyranyl.

In some embodiments, Y² is —(CH₂)ₚ-aryl-*.

In certain embodiments, Y² is —(CH₂)ₚ—(C₅₋₁₂ aryl)-*, and p is 0 or 1.

In certain embodiments, Y² is phenyl or —CH₂-phenyl-*.

In some embodiments, Y² is —(CH₂)ₚ-heteroaryl-*.

In certain embodiments, Y² is —(CH₂)ₚ-(5- to 12-membered heteroaryl)-*, and p is 0 or 1.

In certain embodiments, Y² is pyridinyl, tetrahydroisoquinolinyl, —CH₂-imidazolyl-* or —CH₂-indolyl-*.

In some embodiments, Y³¹ is null or alkyl. In certain embodiments, Y³¹ is null, C₁-6 alkyl, C₁₋₅ alkyl, C₁₋₄ alkyl, C₁₋₃ alkyl or C₁₋₂ alkyl.

In some embodiments, Y³¹ is null, and Y³² is selected from null, —O—#, —C(═O)O—#, —P(═O)(R^{b})—#, —N(R^{b})C(═O)—#, or —C(═O)N(R^{b})—#.

In some embodiments, Y³¹ is null, Y³² is selected from null, —O—#, —C(═O)O—#, —P(═O)(R^{b})—#, —N(R^{b})C(═O)—#, or —C(═O)N(R^{b})—#, and Y³³ is selected from —N(R^{c})₂, alkyl, or aryl, wherein the alkyl and aryl are optionally substituted with one or more R⁷.

In some embodiments, Y³¹ is null, Y³² is selected from null, —O—#, —C(═O)O—#, —P(═O)(R^{b})—#, —N(R^{b})C(═O)—#, or —C(═O)N(R^{b})—#, and Y³³ is selected from —N(R^{c})₂, C₁₋₆ alkyl, or C₅₋₁₂ aryl, wherein the C₁₋₆ alkyl and C₅₋₁₂ aryl are optionally substituted with one or more R⁷.

In certain embodiments, Y³¹ is null, Y³² is selected from null, —O—#, —C(═O)O—#, —P(═O)(R^{b})—#, —N(R^{b})C(═O)—#, or —C(═O)N(R^{b})—#, and Y³³ is selected from —N(R^{c})₂, C₁₋₆ alkyl, or C₅₋₁₂ aryl, wherein the C₁₋₆ alkyl and C₅₋₁₂ aryl are optionally substituted with one or more R⁷ independently selected from halogen, hydroxy, amino, cyano, nitrooxy, or alkyl.

In certain embodiments, Y³¹ is null, Y³² is selected from null, —O—#, —C(═O)O—#, —P(═O)(R^{b})—#, —N(R^{b})C(═O)—#, or —C(═O)N(R^{b})—#, and Y³³ is selected from hydrogen, hydroxyl, cyano, halogen, —NH₂, methyl, —CH₂CH₂OCH₃.

In some embodiments, Y³¹ is alkyl, and Y³² is selected from null, —O—#, —OC(═O)—#, —OC(═O)N(R^{b})—#, or —N(R^{b})C(═O)—#.

In some embodiments, Y³¹ is C₁₋₆ alkyl, C₁₋₅ alkyl, C₁₋₄ alkyl, C₁₋₃ alkyl or C₁₋₂ alkyl, and Y³² is selected from null, —O—#, —OC(═O)—#, —OC(═O)N(R^{b})—#, or —N(R^{b})C(═O)—#.

In some embodiments, Y³¹ is C₁₋₆ alkyl, C₁₋₅ alkyl, C₁₋₄ alkyl, C₁₋₃ alkyl or C₁₋₂ alkyl, Y³² is selected from null, —O—#, —OC(═O)—#, —OC(═O)N(R^{b})—#, or —N(R^{b})C(═O)—#, and Y³³ is selected from —N(R^{c})₂, alkyl, or aryl, wherein the alkyl and aryl are optionally substituted with one or more R⁷.

In some embodiments, Y³¹ is C₁₋₆ alkyl, C₁₋₅ alkyl, C₁₋₄ alkyl, C₁₋₃ alkyl or C₁₋₂ alkyl, Y³² is selected from null, —O—#, —OC(═O)—#, —OC(—O)N(R^{b})—#, or —N(R^{b})C(═O)—#, and Y³³ is selected from —NH₂, —N(CH₃)₂, methyl, dimethylphenyl, or nitrooxypentyl, wherein the alkyl and aryl are optionally substituted with one or more R⁷.

In certain embodiments, Y³¹ is C₁₋₆ alkyl, C₁₋₅ alkyl, C₁₋₄ alkyl, C₁₋₃ alkyl or C₁₋₂ alkyl, Y³² is selected from null, —O—#, —OC(═O)—#, —OC(═O)N(R^{b})—#, or —N(R^{b})C(—O)—#, and Y³³ is selected from —NH₂, —N(CH₃)₂, methyl, dimethylphenyl, or nitrooxypentyl.

In some embodiments, n is 1 or 2.

Exemplary compounds of Formula (I) are set forth in Table 1 below.

TABLE 1
Exemplary Compounds of Formula (I)

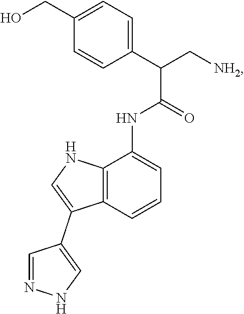 [SMILES: NCC(C(=O)Nc1cccc2c(-c3cn[nH]c3)c[nH]c12)c1ccc(CO)cc1]

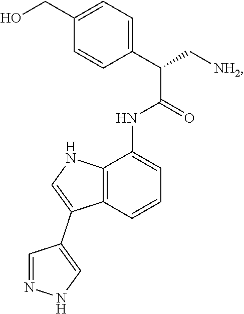 [SMILES: NC[C@H](C(=O)Nc1cccc2c(-c3cn[nH]c3)c[nH]c12)c1ccc(CO)cc1]

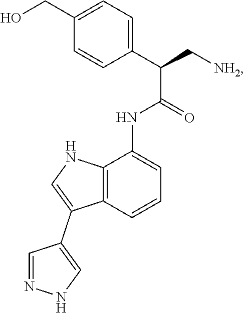 [SMILES: NC[C@@H](C(=O)Nc1cccc2c(-c3cn[nH]c3)c[nH]c12)c1ccc(CO)cc1]

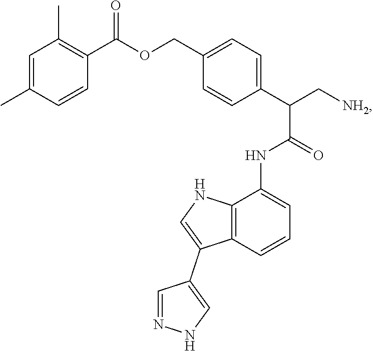 [SMILES: Cc1ccc(C(=O)OCc2ccc(C(CN)C(=O)Nc3cccc4c(-c5cn[nH]c5)c[nH]c34)cc2)c(C)c1]

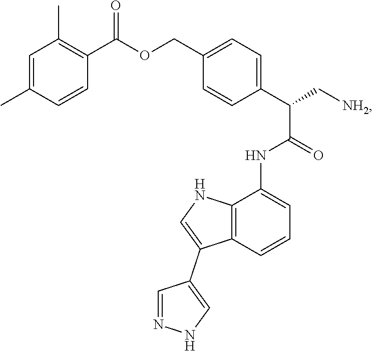 [SMILES: Cc1ccc(C(=O)OCc2ccc([C@H](CN)C(=O)Nc3cccc4c(-c5cn[nH]c5)c[nH]c34)cc2)c(C)c1]

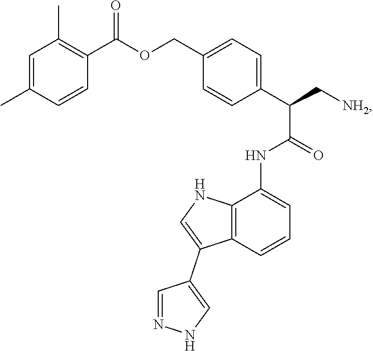 [SMILES: Cc1ccc(C(=O)OCc2ccc([C@@H](CN)C(=O)Nc3cccc4c(-c5cn[nH]c5)c[nH]c34)cc2)c(C)c1]

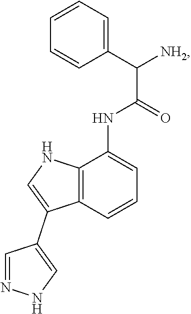 [SMILES: NC(C(=O)Nc1cccc2c(-c3cn[nH]c3)c[nH]c12)c1ccccc1]

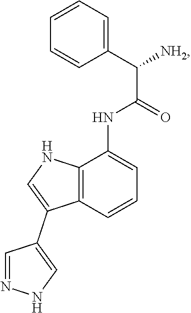 [SMILES: N[C@H](C(=O)Nc1cccc2c(-c3cn[nH]c3)c[nH]c12)c1ccccc1]

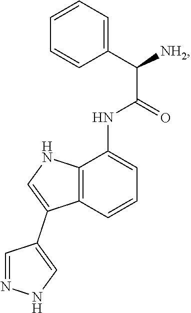 [SMILES: N[C@@H](C(=O)Nc1cccc2c(-c3cn[nH]c3)c[nH]c12)c1ccccc1]

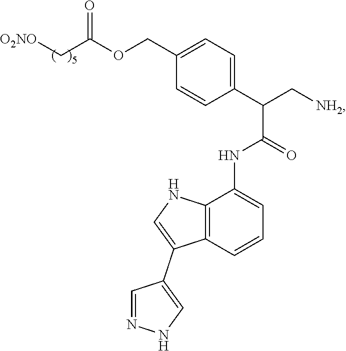

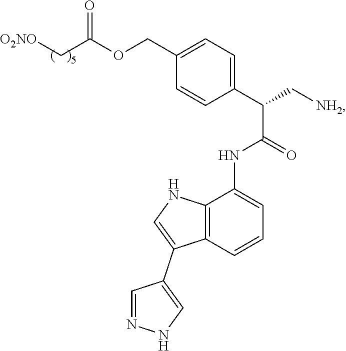

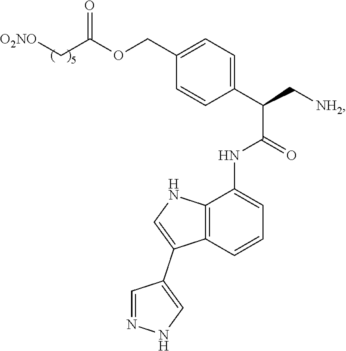

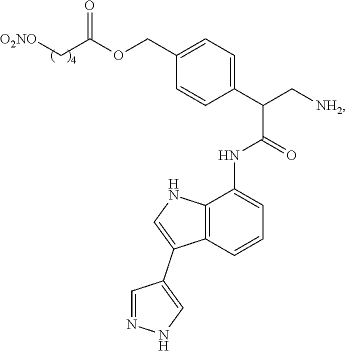

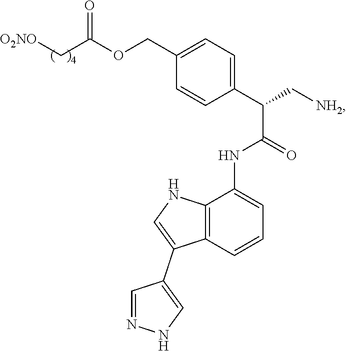

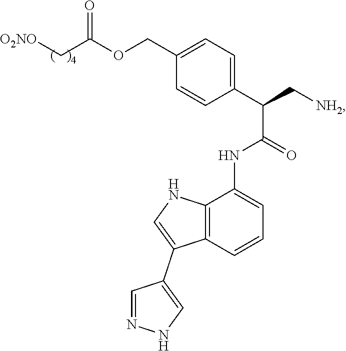

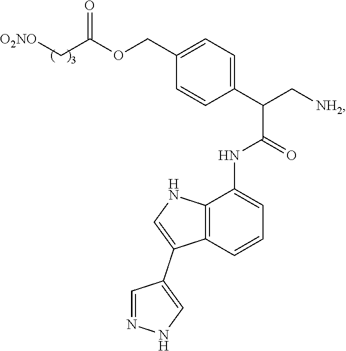

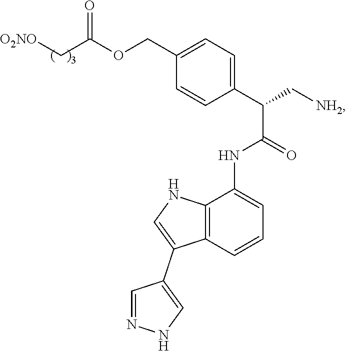

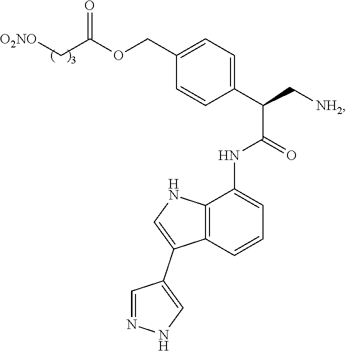

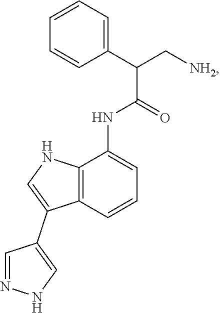 [SMILES: NCC(C(=O)Nc1cccc2c(-c3cn[nH]c3)c[nH]c12)c1ccccc1]

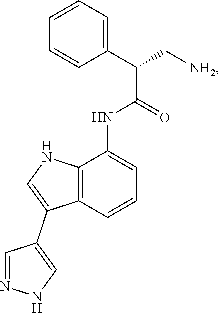 [SMILES: NC[C@H](C(=O)Nc1cccc2c(-c3cn[nH]c3)c[nH]c12)c1ccccc1]

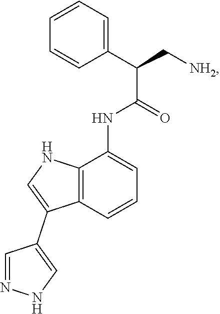 [SMILES: NC[C@@H](C(=O)Nc1cccc2c(-c3cn[nH]c3)c[nH]c12)c1ccccc1]

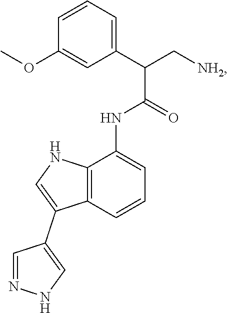 [SMILES: COc1cccc(C(CN)C(=O)Nc2cccc3c(-c4cn[nH]c4)c[nH]c23)c1]

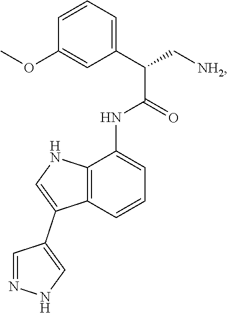 [SMILES: COc1cccc([C@H](CN)C(=O)Nc2cccc3c(-c4cn[nH]c4)c[nH]c23)c1]

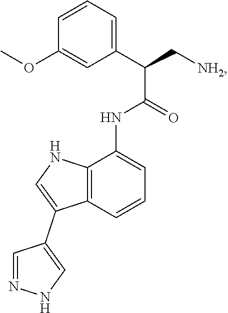 [SMILES: COc1cccc([C@@H](CN)C(=O)Nc2cccc3c(-c4cn[nH]c4)c[nH]c23)c1]

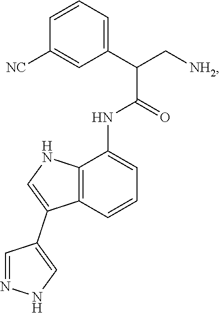 [SMILES: N#Cc1cccc(C(CN)C(=O)Nc2cccc3c(-c4cn[nH]c4)c[nH]c23)c1]

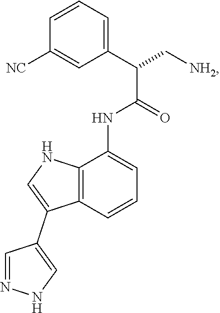 [SMILES: N#Cc1cccc([C@H](CN)C(=O)Nc2cccc3c(-c4cn[nH]c4)c[nH]c23)c1]

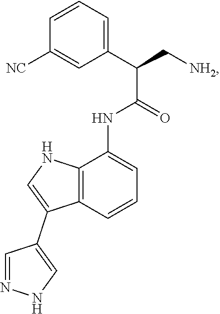 [SMILES: N#Cc1cccc([C@@H](CN)C(=O)Nc2cccc3c(-c4cn[nH]c4)c[nH]c23)c1]

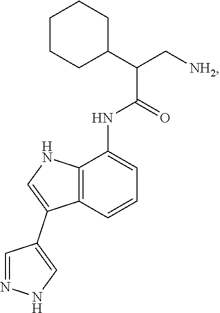 [SMILES: NCC(C(=O)Nc1cccc2c(-c3cn[nH]c3)c[nH]c12)C1CCCCC1]

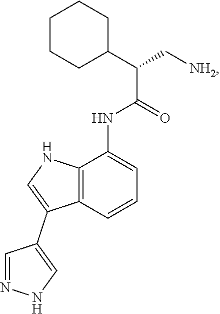 [SMILES: NC[C@H](C(=O)Nc1cccc2c(-c3cn[nH]c3)c[nH]c12)C1CCCCC1]

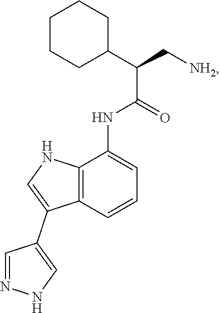 [SMILES: NC[C@@H](C(=O)Nc1cccc2c(-c3cn[nH]c3)c[nH]c12)C1CCCCC1]

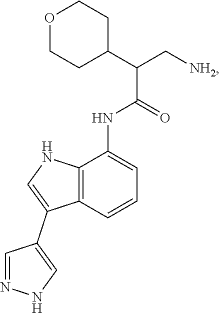 [SMILES: NCC(C(=O)Nc1cccc2c(-c3cn[nH]c3)c[nH]c12)C1CCOCC1]

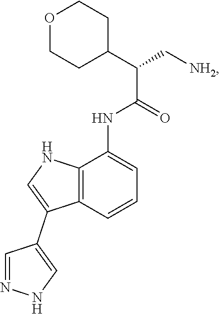 [SMILES: NC[C@H](C(=O)Nc1cccc2c(-c3cn[nH]c3)c[nH]c12)C1CCOCC1]

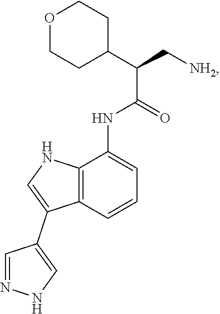 [SMILES: NC[C@@H](C(=O)Nc1cccc2c(-c3cn[nH]c3)c[nH]c12)C1CCOCC1]

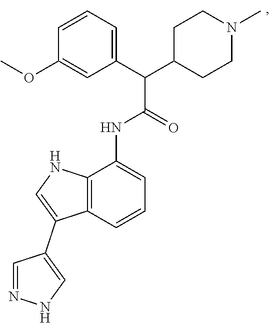

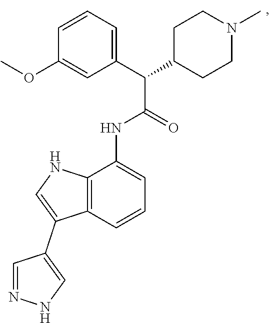

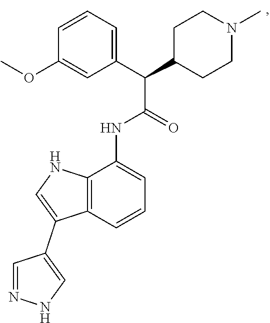

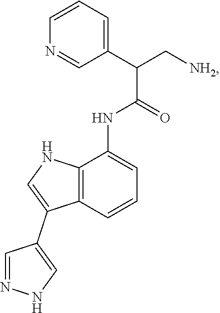 [SMILES: NCC(C(=O)Nc1cccc2c(-c3cn[nH]c3)c[nH]c12)c1cccnc1]

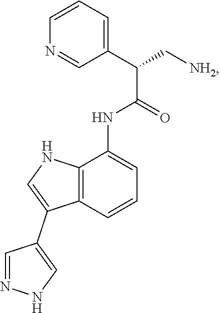 [SMILES: NC[C@H](C(=O)Nc1cccc2c(-c3cn[nH]c3)c[nH]c12)c1cccnc1]

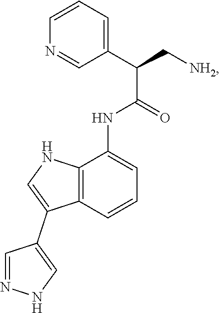 [SMILES: NC[C@@H](C(=O)Nc1cccc2c(-c3cn[nH]c3)c[nH]c12)c1cccnc1]

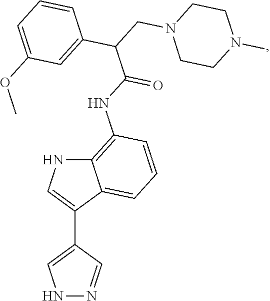

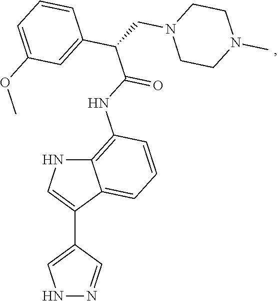 [SMILES: COc1cccc([C@H](CN2CCN(C)CC2)C(=O)Nc2cccc3c(-c4cn[nH]c4)c[nH]c23)c1]

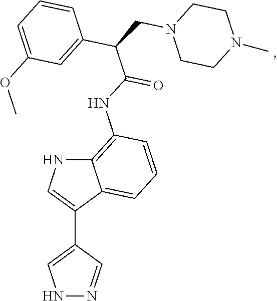 [SMILES: COc1cccc([C@@H](CN2CCN(C)CC2)C(=O)Nc2cccc3c(-c4cn[nH]c4)c[nH]c23)c1]

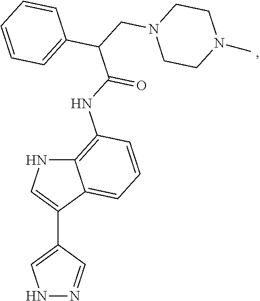 [SMILES: CN1CCN(CC(C(=O)Nc2cccc3c(-c4cn[nH]c4)c[nH]c23)c2ccccc2)CC1]

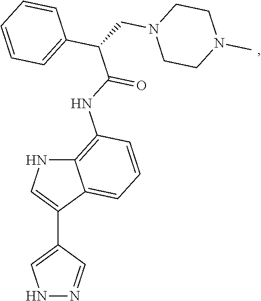 [SMILES: CN1CCN(C[C@H](C(=O)Nc2cccc3c(-c4cn[nH]c4)c[nH]c23)c2ccccc2)CC1]

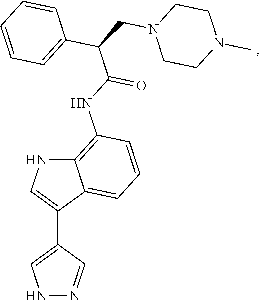 [SMILES: CN1CCN(C[C@@H](C(=O)Nc2cccc3c(-c4cn[nH]c4)c[nH]c23)c2ccccc2)CC1]

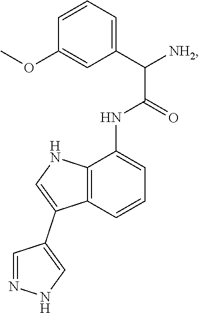 [SMILES: COc1cccc(C(N)C(=O)Nc2cccc3c(-c4cn[nH]c4)c[nH]c23)c1]

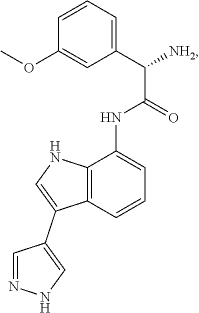 [SMILES: COc1cccc([C@H](N)C(=O)Nc2cccc3c(-c4cn[nH]c4)c[nH]c23)c1]

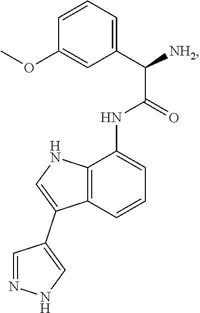 [SMILES: COc1cccc([C@@H](N)C(=O)Nc2cccc3c(-c4cn[nH]c4)c[nH]c23)c1]

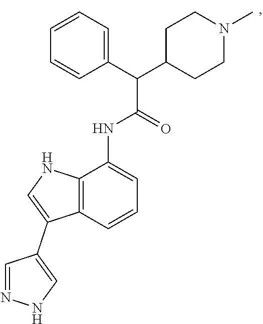 [SMILES: CN1CCC(C(C(=O)Nc2cccc3c(-c4cn[nH]c4)c[nH]c23)c2ccccc2)CC1]

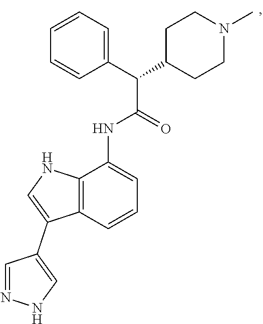 [SMILES: CN1CCC([C@H](C(=O)Nc2cccc3c(-c4cn[nH]c4)c[nH]c23)c2ccccc2)CC1]

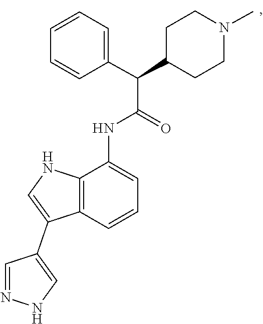 [SMILES: CN1CCC([C@@H](C(=O)Nc2cccc3c(-c4cn[nH]c4)c[nH]c23)c2ccccc2)CC1]

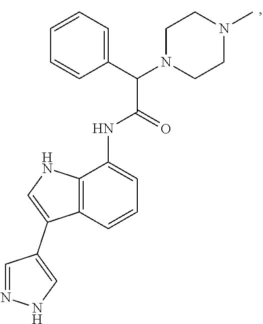 [SMILES: CN1CCN(C(C(=O)Nc2cccc3c(-c4cn[nH]c4)c[nH]c23)c2ccccc2)CC1]

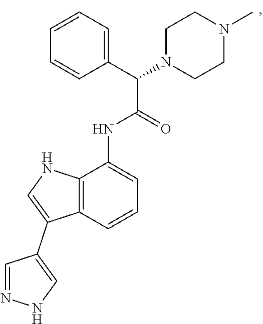 [SMILES: CN1CCN([C@H](C(=O)Nc2cccc3c(-c4cn[nH]c4)c[nH]c23)c2ccccc2)CC1]

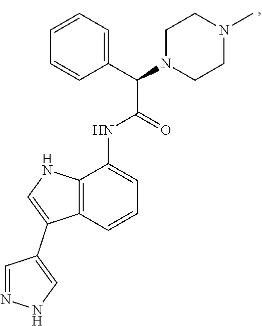 [SMILES: CN1CCN([C@@H](C(=O)Nc2cccc3c(-c4cn[nH]c4)c[nH]c23)c2ccccc2)CC1]

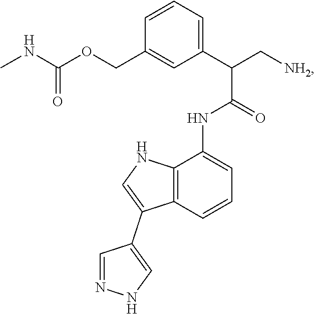 [SMILES: CNC(=O)OCc1cccc(C(CN)C(=O)Nc2cccc3c(-c4cn[nH]c4)c[nH]c23)c1]

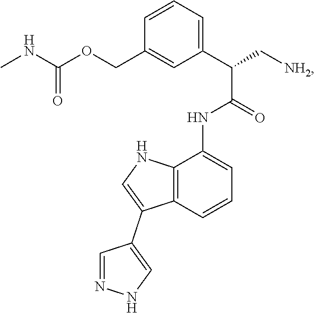 [SMILES: CNC(=O)OCc1cccc([C@H](CN)C(=O)Nc2cccc3c(-c4cn[nH]c4)c[nH]c23)c1]

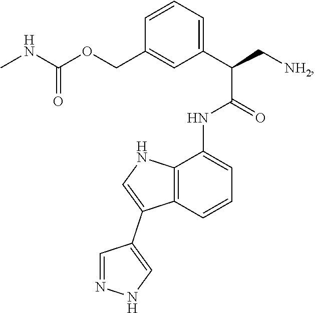 [SMILES: CNC(=O)OCc1cccc([C@@H](CN)C(=O)Nc2cccc3c(-c4cn[nH]c4)c[nH]c23)c1]

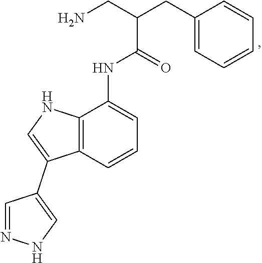 [SMILES: NCC(Cc1ccccc1)C(=O)Nc1cccc2c(-c3cn[nH]c3)c[nH]c12]

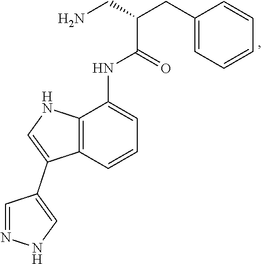 [SMILES: NC[C@H](Cc1ccccc1)C(=O)Nc1cccc2c(-c3cn[nH]c3)c[nH]c12]

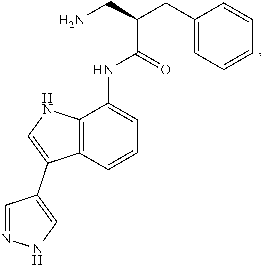 [SMILES: NC[C@@H](Cc1ccccc1)C(=O)Nc1cccc2c(-c3cn[nH]c3)c[nH]c12]

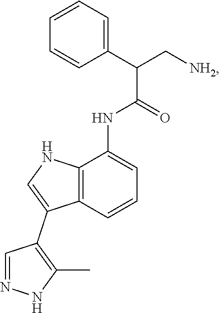 [SMILES: Cc1[nH]ncc1-c1c[nH]c2c(NC(=O)C(CN)c3ccccc3)cccc12]

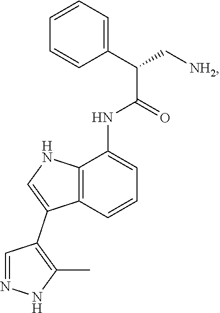 [SMILES: Cc1[nH]ncc1-c1c[nH]c2c(NC(=O)[C@@H](CN)c3ccccc3)cccc12]

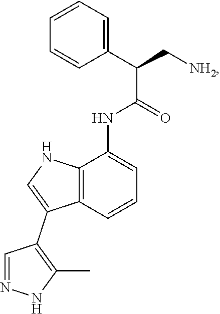 [SMILES: Cc1[nH]ncc1-c1c[nH]c2c(NC(=O)[C@H](CN)c3ccccc3)cccc12]

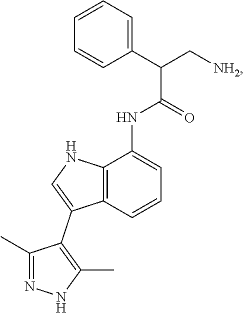 [SMILES: Cc1n[nH]c(C)c1-c1c[nH]c2c(NC(=O)C(CN)c3ccccc3)cccc12]

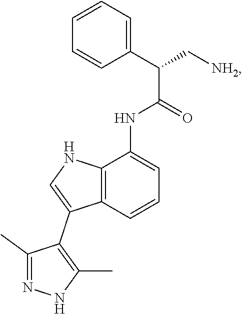 [SMILES: Cc1n[nH]c(C)c1-c1c[nH]c2c(NC(=O)[C@@H](CN)c3ccccc3)cccc12]

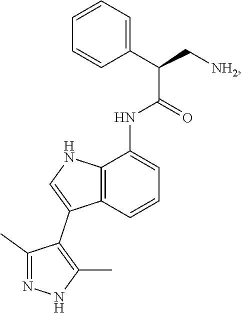 [SMILES: Cc1n[nH]c(C)c1-c1c[nH]c2c(NC(=O)[C@H](CN)c3ccccc3)cccc12]

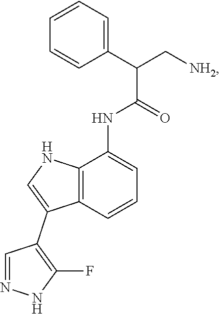 [SMILES: NCC(C(=O)Nc1cccc2c(-c3cn[nH]c3F)c[nH]c12)c1ccccc1]

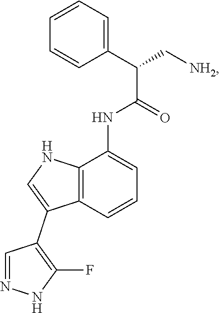 [SMILES: NC[C@H](C(=O)Nc1cccc2c(-c3cn[nH]c3F)c[nH]c12)c1ccccc1]

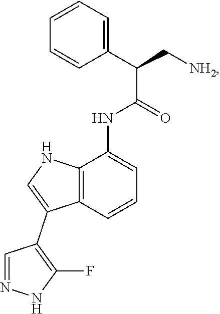 [SMILES: NC[C@@H](C(=O)Nc1cccc2c(-c3cn[nH]c3F)c[nH]c12)c1ccccc1]

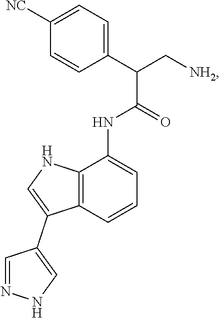 [SMILES: N#Cc1ccc(C(CN)C(=O)Nc2cccc3c(-c4cn[nH]c4)c[nH]c23)cc1]

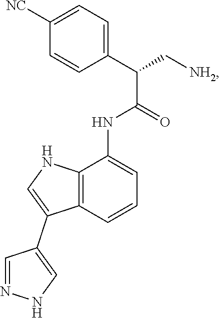 [SMILES: N#Cc1ccc([C@H](CN)C(=O)Nc2cccc3c(-c4cn[nH]c4)c[nH]c23)cc1]

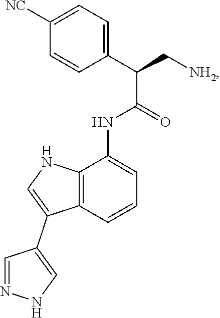 [SMILES: N#Cc1ccc([C@@H](CN)C(=O)Nc2cccc3c(-c4cn[nH]c4)c[nH]c23)cc1]

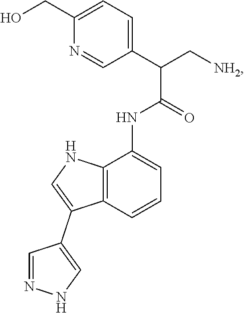 [SMILES: NCC(C(=O)Nc1cccc2c(-c3cn[nH]c3)c[nH]c12)c1ccc(CO)nc1]

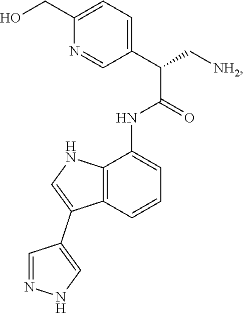 [SMILES: NC[C@H](C(=O)Nc1cccc2c(-c3cn[nH]c3)c[nH]c12)c1ccc(CO)nc1]

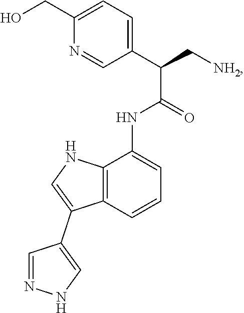 [SMILES: NC[C@@H](C(=O)Nc1cccc2c(-c3cn[nH]c3)c[nH]c12)c1ccc(CO)nc1]

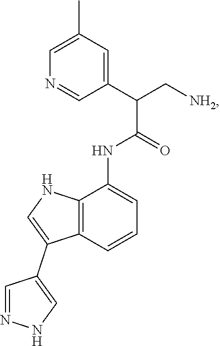 [SMILES: Cc1cncc(C(CN)C(=O)Nc2cccc3c(-c4cn[nH]c4)c[nH]c23)c1]

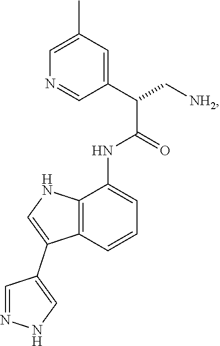 [SMILES: Cc1cncc([C@H](CN)C(=O)Nc2cccc3c(-c4cn[nH]c4)c[nH]c23)c1]

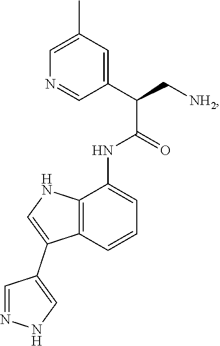 [SMILES: Cc1cncc([C@@H](CN)C(=O)Nc2cccc3c(-c4cn[nH]c4)c[nH]c23)c1]

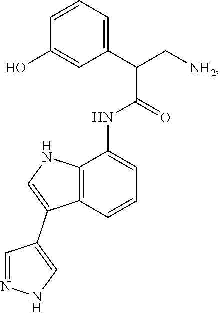 [SMILES: NCC(C(=O)Nc1cccc2c(-c3cn[nH]c3)c[nH]c12)c1cccc(O)c1]

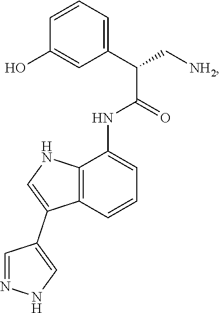 [SMILES: NC[C@H](C(=O)Nc1cccc2c(-c3cn[nH]c3)c[nH]c12)c1cccc(O)c1]

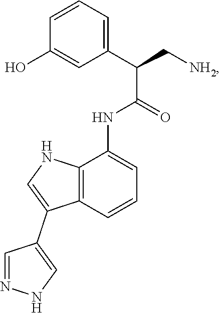 [SMILES: NC[C@@H](C(=O)Nc1cccc2c(-c3cn[nH]c3)c[nH]c12)c1cccc(O)c1]

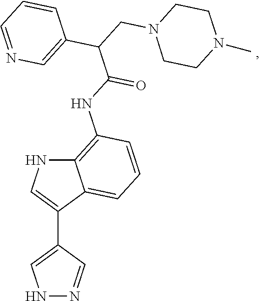 [SMILES: CN1CCN(CC(C(=O)Nc2cccc3c(-c4cn[nH]c4)c[nH]c23)c2cccnc2)CC1]

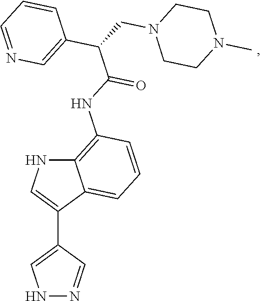 [SMILES: CN1CCN(C[C@H](C(=O)Nc2cccc3c(-c4cn[nH]c4)c[nH]c23)c2cccnc2)CC1]

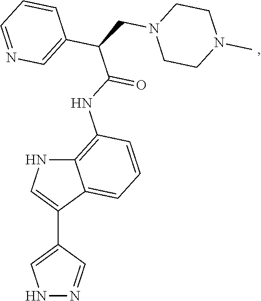 [SMILES: CN1CCN(C[C@@H](C(=O)Nc2cccc3c(-c4cn[nH]c4)c[nH]c23)c2cccnc2)CC1]

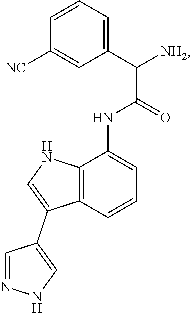 [SMILES: N#Cc1cccc(C(N)C(=O)Nc2cccc3c(-c4cn[nH]c4)c[nH]c23)c1]

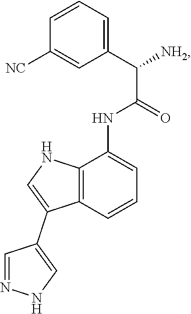 [SMILES: N#Cc1cccc([C@H](N)C(=O)Nc2cccc3c(-c4cn[nH]c4)c[nH]c23)c1]

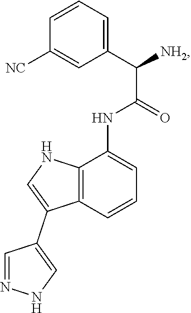 [SMILES: N#Cc1cccc([C@@H](N)C(=O)Nc2cccc3c(-c4cn[nH]c4)c[nH]c23)c1]

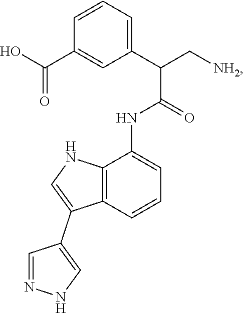 [SMILES: NCC(C(=O)Nc1cccc2c(-c3cn[nH]c3)c[nH]c12)c1cccc(C(=O)O)c1]

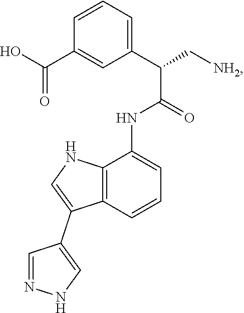 [SMILES: NC[C@H](C(=O)Nc1cccc2c(-c3cn[nH]c3)c[nH]c12)c1cccc(C(=O)O)c1]

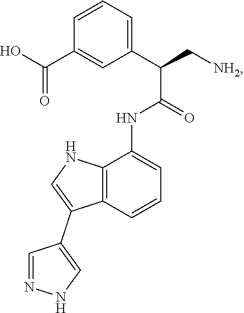 [SMILES: NC[C@@H](C(=O)Nc1cccc2c(-c3cn[nH]c3)c[nH]c12)c1cccc(C(=O)O)c1]

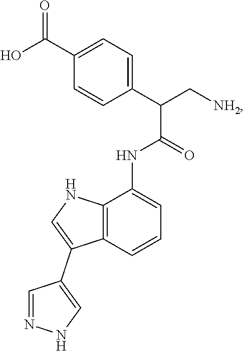 [SMILES: NCC(C(=O)Nc1cccc2c(-c3cn[nH]c3)c[nH]c12)c1ccc(C(=O)O)cc1]

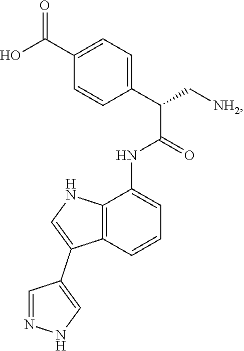 [SMILES: NC[C@H](C(=O)Nc1cccc2c(-c3cn[nH]c3)c[nH]c12)c1ccc(C(=O)O)cc1]

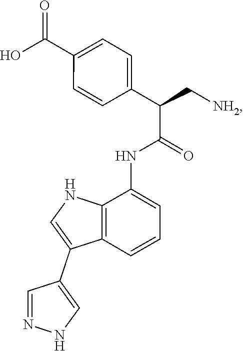 [SMILES: NC[C@@H](C(=O)Nc1cccc2c(-c3cn[nH]c3)c[nH]c12)c1ccc(C(=O)O)cc1]

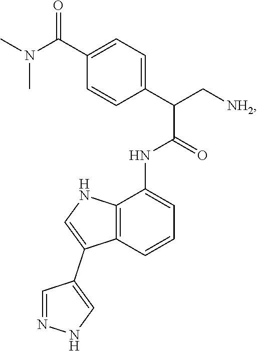 [SMILES: CN(C)C(=O)c1ccc(C(CN)C(=O)Nc2cccc3c(-c4cn[nH]c4)c[nH]c23)cc1]

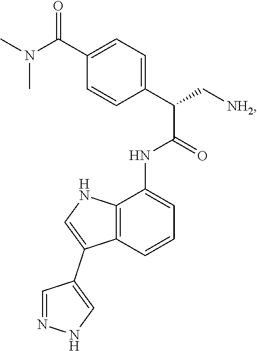 [SMILES: CN(C)C(=O)c1ccc([C@H](CN)C(=O)Nc2cccc3c(-c4cn[nH]c4)c[nH]c23)cc1]

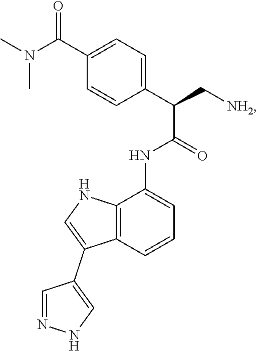 [SMILES: CN(C)C(=O)c1ccc([C@@H](CN)C(=O)Nc2cccc3c(-c4cn[nH]c4)c[nH]c23)cc1]

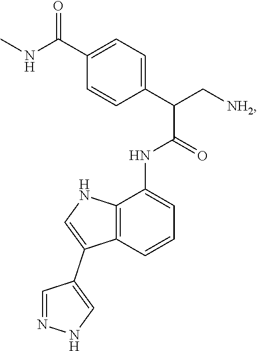 [SMILES: CNC(=O)c1ccc(C(CN)C(=O)Nc2cccc3c(-c4cn[nH]c4)c[nH]c23)cc1]

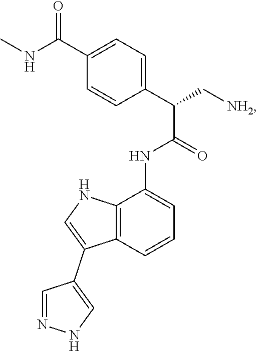 [SMILES: CNC(=O)c1ccc([C@H](CN)C(=O)Nc2cccc3c(-c4cn[nH]c4)c[nH]c23)cc1]

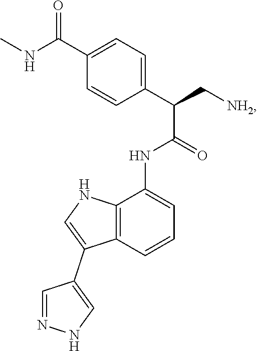 [SMILES: CNC(=O)c1ccc([C@@H](CN)C(=O)Nc2cccc3c(-c4cn[nH]c4)c[nH]c23)cc1]

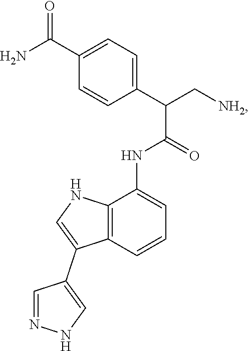 [SMILES: NCC(C(=O)Nc1cccc2c(-c3cn[nH]c3)c[nH]c12)c1ccc(C(N)=O)cc1]

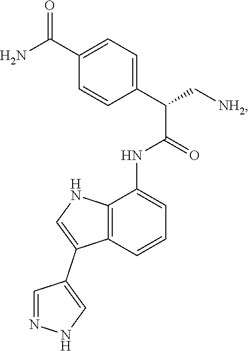 [SMILES: NC[C@H](C(=O)Nc1cccc2c(-c3cn[nH]c3)c[nH]c12)c1ccc(C(N)=O)cc1]

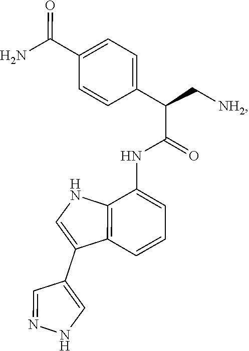 [SMILES: NC[C@@H](C(=O)Nc1cccc2c(-c3cn[nH]c3)c[nH]c12)c1ccc(C(N)=O)cc1]

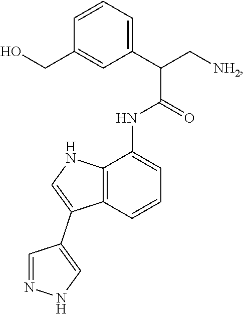 [SMILES: NCC(C(=O)Nc1cccc2c(-c3cn[nH]c3)c[nH]c12)c1cccc(CO)c1]

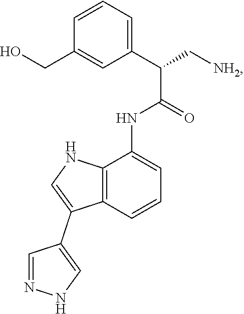 [SMILES: NC[C@H](C(=O)Nc1cccc2c(-c3cn[nH]c3)c[nH]c12)c1cccc(CO)c1]

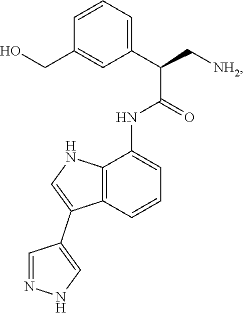 [SMILES: NC[C@@H](C(=O)Nc1cccc2c(-c3cn[nH]c3)c[nH]c12)c1cccc(CO)c1]

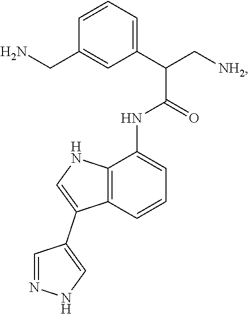 [SMILES: NCc1cccc(C(CN)C(=O)Nc2cccc3c(-c4cn[nH]c4)c[nH]c23)c1]

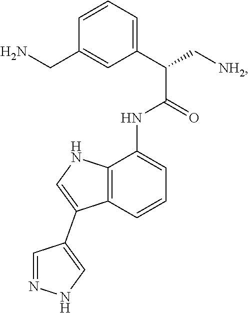 [SMILES: NCc1cccc([C@H](CN)C(=O)Nc2cccc3c(-c4cn[nH]c4)c[nH]c23)c1]

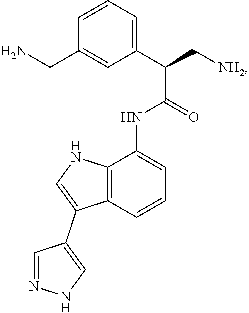 [SMILES: NCc1cccc([C@@H](CN)C(=O)Nc2cccc3c(-c4cn[nH]c4)c[nH]c23)c1]

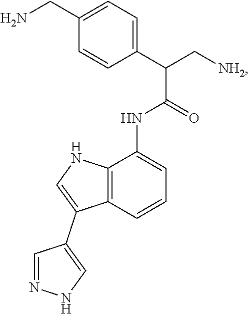 [SMILES: NCc1ccc(C(CN)C(=O)Nc2cccc3c(-c4cn[nH]c4)c[nH]c23)cc1]

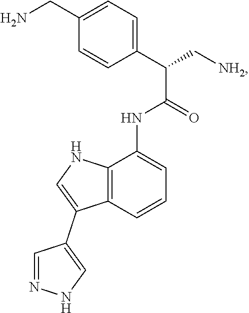 [SMILES: NCc1ccc([C@H](CN)C(=O)Nc2cccc3c(-c4cn[nH]c4)c[nH]c23)cc1]

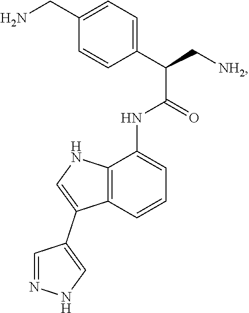 [SMILES: NCc1ccc([C@@H](CN)C(=O)Nc2cccc3c(-c4cn[nH]c4)c[nH]c23)cc1]

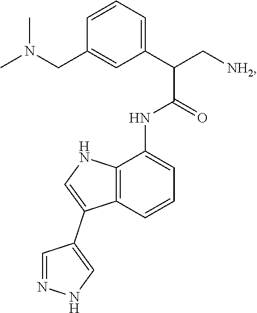 [SMILES: CN(C)Cc1cccc(C(CN)C(=O)Nc2cccc3c(-c4cn[nH]c4)c[nH]c23)c1]

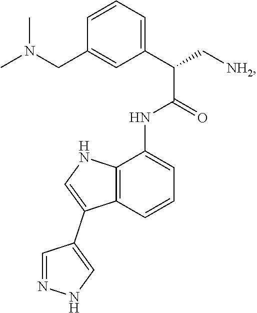 [SMILES: CN(C)Cc1cccc([C@H](CN)C(=O)Nc2cccc3c(-c4cn[nH]c4)c[nH]c23)c1]

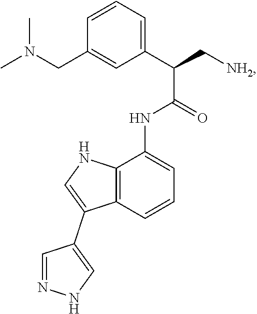 [SMILES: CN(C)Cc1cccc([C@@H](CN)C(=O)Nc2cccc3c(-c4cn[nH]c4)c[nH]c23)c1]

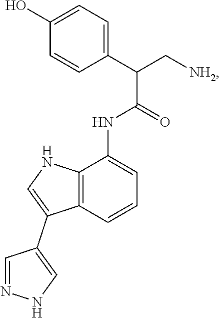 [SMILES: NCC(C(=O)Nc1cccc2c(-c3cn[nH]c3)c[nH]c12)c1ccc(O)cc1]

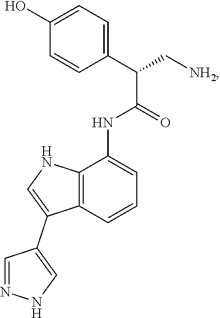 [SMILES: NC[C@H](C(=O)Nc1cccc2c(-c3cn[nH]c3)c[nH]c12)c1ccc(O)cc1]

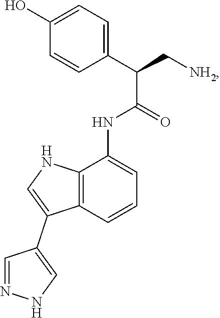 [SMILES: NC[C@@H](C(=O)Nc1cccc2c(-c3cn[nH]c3)c[nH]c12)c1ccc(O)cc1]

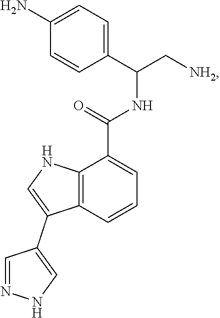 [SMILES: NCC(NC(=O)c1cccc2c(-c3cn[nH]c3)c[nH]c12)c1ccc(N)cc1]

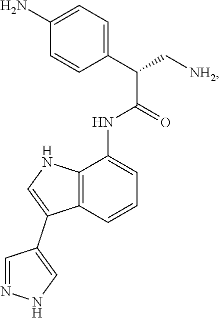 [SMILES: NC[C@H](C(=O)Nc1cccc2c(-c3cn[nH]c3)c[nH]c12)c1ccc(N)cc1]

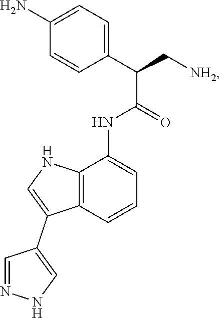 [SMILES: NC[C@@H](C(=O)Nc1cccc2c(-c3cn[nH]c3)c[nH]c12)c1ccc(N)cc1]

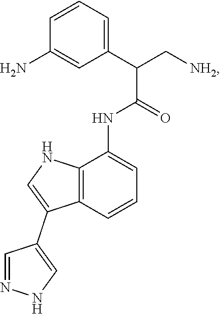 [SMILES: NCC(C(=O)Nc1cccc2c(-c3cn[nH]c3)c[nH]c12)c1cccc(N)c1]

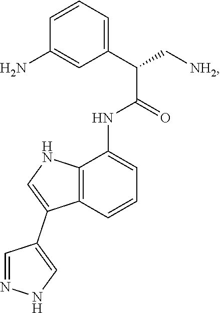 [SMILES: NC[C@H](C(=O)Nc1cccc2c(-c3cn[nH]c3)c[nH]c12)c1cccc(N)c1]

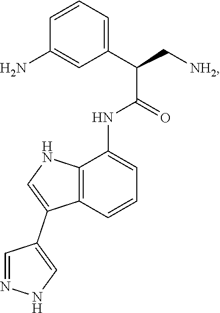 [SMILES: NC[C@@H](C(=O)Nc1cccc2c(-c3cn[nH]c3)c[nH]c12)c1cccc(N)c1]

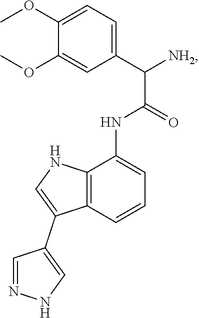 [SMILES: COc1ccc(C(N)C(=O)Nc2cccc3c(-c4cn[nH]c4)c[nH]c23)cc1OC]

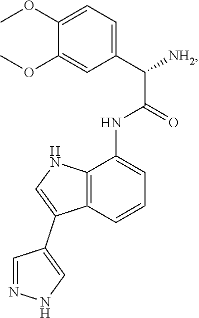 [SMILES: COc1ccc([C@H](N)C(=O)Nc2cccc3c(-c4cn[nH]c4)c[nH]c23)cc1OC]

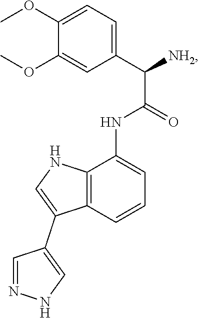 [SMILES: COc1ccc([C@@H](N)C(=O)Nc2cccc3c(-c4cn[nH]c4)c[nH]c23)cc1OC]

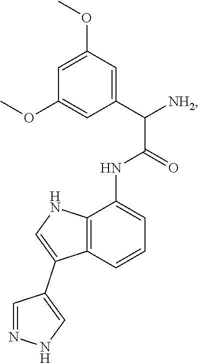 [SMILES: COc1cc(OC)cc(C(N)C(=O)Nc2cccc3c(-c4cn[nH]c4)c[nH]c23)c1]

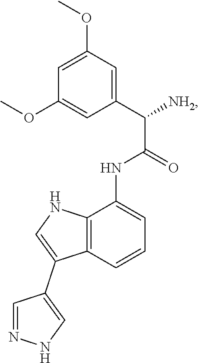 [SMILES: COc1cc(OC)cc([C@H](N)C(=O)Nc2cccc3c(-c4cn[nH]c4)c[nH]c23)c1]

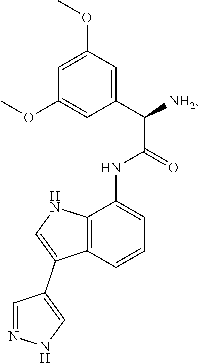 [SMILES: COc1cc(OC)cc([C@@H](N)C(=O)Nc2cccc3c(-c4cn[nH]c4)c[nH]c23)c1]

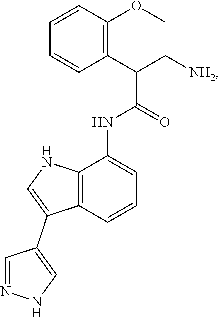 [SMILES: COc1ccccc1C(CN)C(=O)Nc1cccc2c(-c3cn[nH]c3)c[nH]c12]

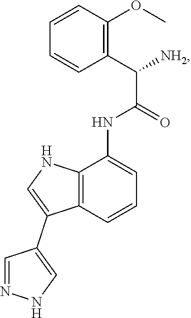 [SMILES: COc1ccccc1[C@H](N)C(=O)Nc1cccc2c(-c3cn[nH]c3)c[nH]c12]

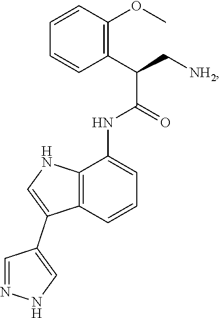 [SMILES: COc1ccccc1[C@@H](CN)C(=O)Nc1cccc2c(-c3cn[nH]c3)c[nH]c12]

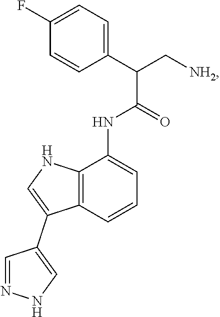 [SMILES: NCC(C(=O)Nc1cccc2c(-c3cn[nH]c3)c[nH]c12)c1ccc(F)cc1]

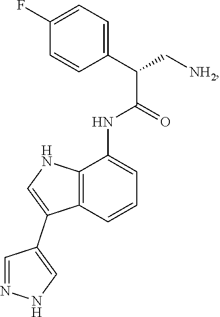 [SMILES: NC[C@H](C(=O)Nc1cccc2c(-c3cn[nH]c3)c[nH]c12)c1ccc(F)cc1]

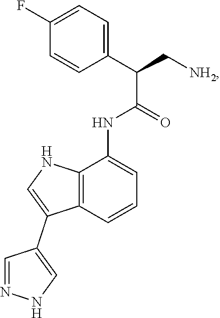 [SMILES: NC[C@@H](C(=O)Nc1cccc2c(-c3cn[nH]c3)c[nH]c12)c1ccc(F)cc1]

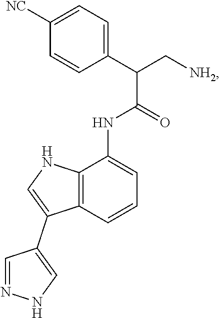 [SMILES: N#Cc1ccc(C(CN)C(=O)Nc2cccc3c(-c4cn[nH]c4)c[nH]c23)cc1]

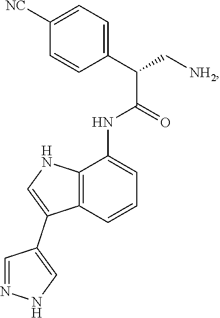 [SMILES: N#Cc1ccc([C@H](CN)C(=O)Nc2cccc3c(-c4cn[nH]c4)c[nH]c23)cc1]

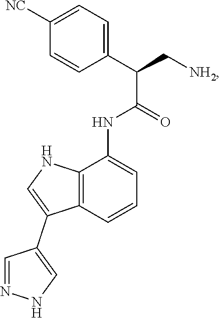 [SMILES: N#Cc1ccc([C@@H](CN)C(=O)Nc2cccc3c(-c4cn[nH]c4)c[nH]c23)cc1]

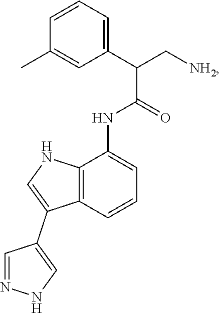 [SMILES: Cc1cccc(C(CN)C(=O)Nc2cccc3c(-c4cn[nH]c4)c[nH]c23)c1]

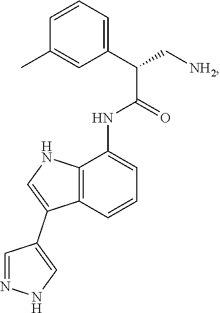 [SMILES: Cc1cccc([C@H](CN)C(=O)Nc2cccc3c(-c4cn[nH]c4)c[nH]c23)c1]

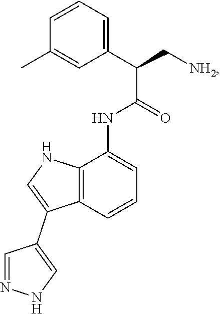 [SMILES: Cc1cccc([C@@H](CN)C(=O)Nc2cccc3c(-c4cn[nH]c4)c[nH]c23)c1]

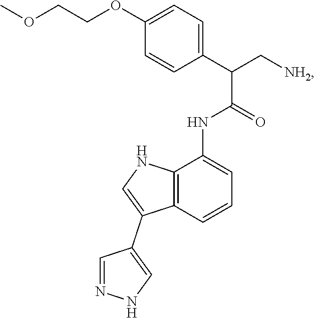 [SMILES: COCCOc1ccc(C(CN)C(=O)Nc2cccc3c(-c4cn[nH]c4)c[nH]c23)cc1]

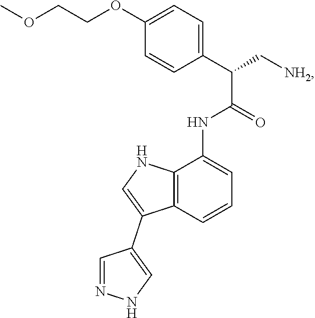 [SMILES: COCCOc1ccc([C@H](CN)C(=O)Nc2cccc3c(-c4cn[nH]c4)c[nH]c23)cc1]

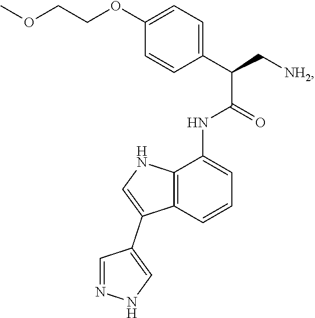 [SMILES: COCCOc1ccc([C@@H](CN)C(=O)Nc2cccc3c(-c4cn[nH]c4)c[nH]c23)cc1]

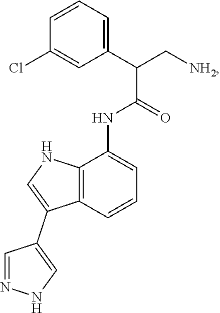 [SMILES: NCC(C(=O)Nc1cccc2c(-c3cn[nH]c3)c[nH]c12)c1cccc(Cl)c1]

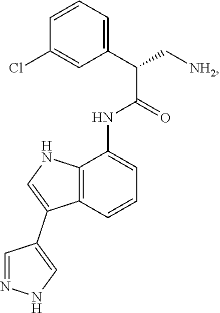 [SMILES: NC[C@H](C(=O)Nc1cccc2c(-c3cn[nH]c3)c[nH]c12)c1cccc(Cl)c1]

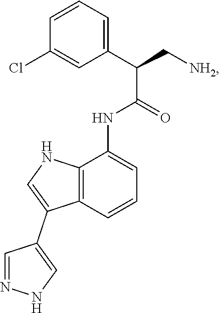 [SMILES: NC[C@@H](C(=O)Nc1cccc2c(-c3cn[nH]c3)c[nH]c12)c1cccc(Cl)c1]

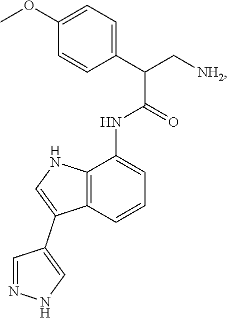 [SMILES: COc1ccc(C(CN)C(=O)Nc2cccc3c(-c4cn[nH]c4)c[nH]c23)cc1]

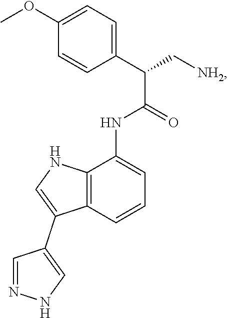 [SMILES: COc1ccc([C@H](CN)C(=O)Nc2cccc3c(-c4cn[nH]c4)c[nH]c23)cc1]

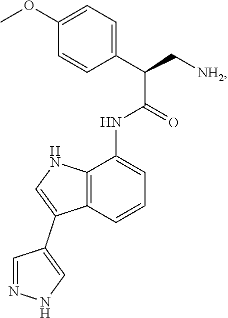 [SMILES: COc1ccc([C@@H](CN)C(=O)Nc2cccc3c(-c4cn[nH]c4)c[nH]c23)cc1]

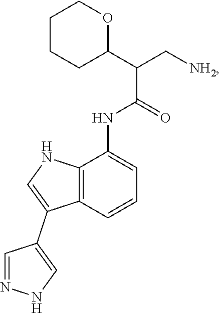 [SMILES: NCC(C(=O)Nc1cccc2c(-c3cn[nH]c3)c[nH]c12)C1CCCCO1]

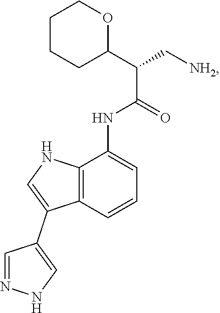 [SMILES: NC[C@H](C(=O)Nc1cccc2c(-c3cn[nH]c3)c[nH]c12)C1CCCCO1]

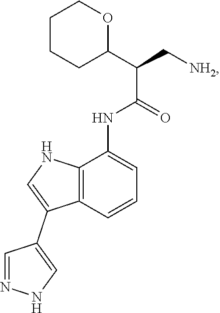 [SMILES: NC[C@@H](C(=O)Nc1cccc2c(-c3cn[nH]c3)c[nH]c12)C1CCCCO1]

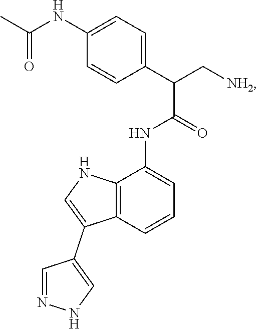 [SMILES: CC(=O)Nc1ccc(C(CN)C(=O)Nc2cccc3c(-c4cn[nH]c4)c[nH]c23)cc1]

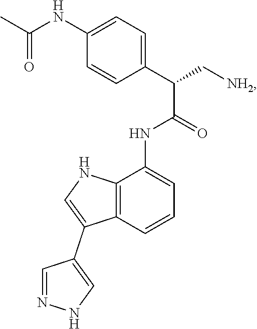 [SMILES: CC(=O)Nc1ccc([C@H](CN)C(=O)Nc2cccc3c(-c4cn[nH]c4)c[nH]c23)cc1]

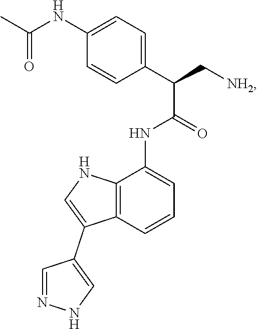 [SMILES: CC(=O)Nc1ccc([C@@H](CN)C(=O)Nc2cccc3c(-c4cn[nH]c4)c[nH]c23)cc1]

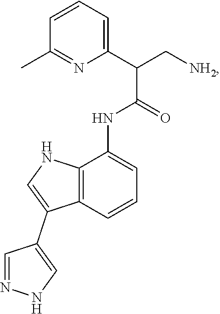 [SMILES: Cc1cccc(C(CN)C(=O)Nc2cccc3c(-c4cn[nH]c4)c[nH]c23)n1]

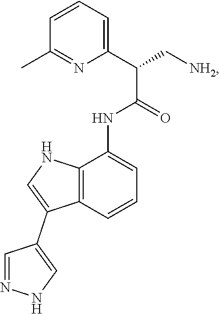 [SMILES: Cc1cccc([C@H](CN)C(=O)Nc2cccc3c(-c4cn[nH]c4)c[nH]c23)n1]

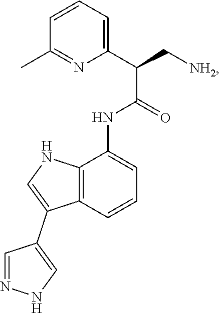 [SMILES: Cc1cccc([C@@H](CN)C(=O)Nc2cccc3c(-c4cn[nH]c4)c[nH]c23)n1]

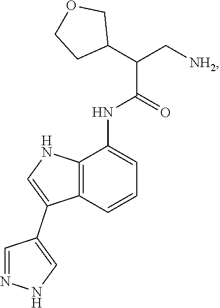 [SMILES: NCC(C(=O)Nc1cccc2c(-c3cn[nH]c3)c[nH]c12)C1CCOC1]

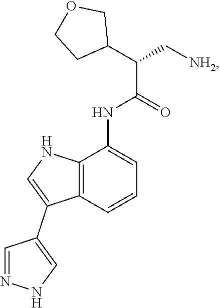 [SMILES: NC[C@H](C(=O)Nc1cccc2c(-c3cn[nH]c3)c[nH]c12)C1CCOC1]

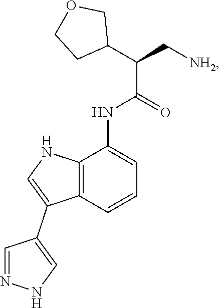 [SMILES: NC[C@@H](C(=O)Nc1cccc2c(-c3cn[nH]c3)c[nH]c12)C1CCOC1]

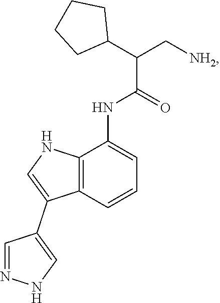 [SMILES: NCC(C(=O)Nc1cccc2c(-c3cn[nH]c3)c[nH]c12)C1CCCC1]

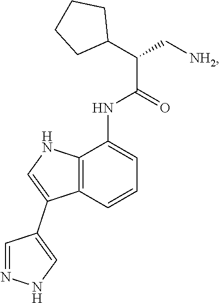 [SMILES: NC[C@H](C(=O)Nc1cccc2c(-c3cn[nH]c3)c[nH]c12)C1CCCC1]

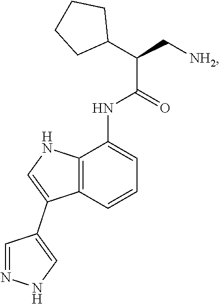 [SMILES: NC[C@@H](C(=O)Nc1cccc2c(-c3cn[nH]c3)c[nH]c12)C1CCCC1]

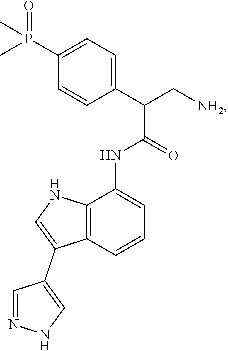 [SMILES: CP(C)(=O)c1ccc(C(CN)C(=O)Nc2cccc3c(-c4cn[nH]c4)c[nH]c23)cc1]

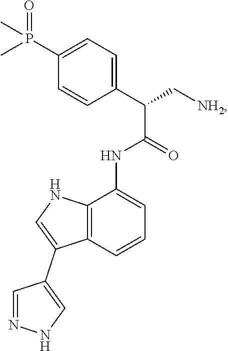 [SMILES: CP(C)(=O)c1ccc([C@H](CN)C(=O)Nc2cccc3c(-c4cn[nH]c4)c[nH]c23)cc1]

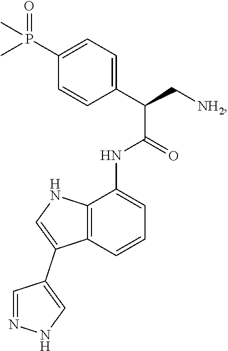 [SMILES: CP(C)(=O)c1ccc([C@@H](CN)C(=O)Nc2cccc3c(-c4cn[nH]c4)c[nH]c23)cc1]

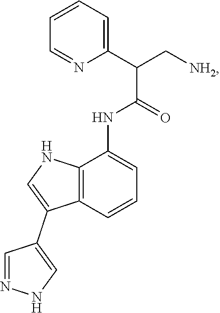 [SMILES: NCC(C(=O)Nc1cccc2c(-c3cn[nH]c3)c[nH]c12)c1ccccn1]

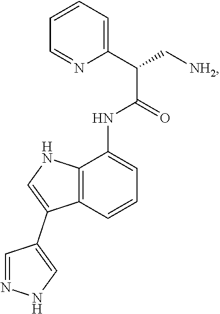 [SMILES: NC[C@H](C(=O)Nc1cccc2c(-c3cn[nH]c3)c[nH]c12)c1ccccn1]

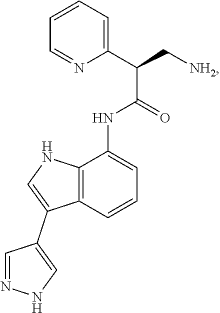 [SMILES: NC[C@@H](C(=O)Nc1cccc2c(-c3cn[nH]c3)c[nH]c12)c1ccccn1]

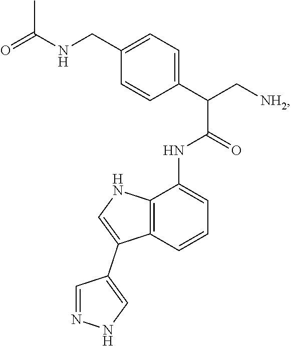 [SMILES: CC(=O)NCc1ccc(C(CN)C(=O)Nc2cccc3c(-c4cn[nH]c4)c[nH]c23)cc1]

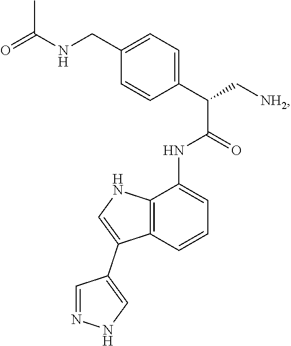 [SMILES: CC(=O)NCc1ccc([C@H](CN)C(=O)Nc2cccc3c(-c4cn[nH]c4)c[nH]c23)cc1]

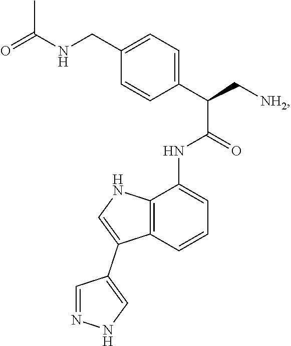 [SMILES: CC(=O)NCc1ccc([C@@H](CN)C(=O)Nc2cccc3c(-c4cn[nH]c4)c[nH]c23)cc1]

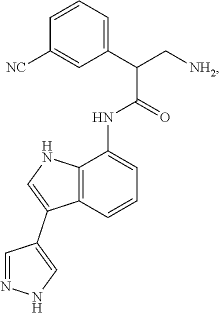 [SMILES: N#Cc1cccc(C(CN)C(=O)Nc2cccc3c(-c4cn[nH]c4)c[nH]c23)c1]

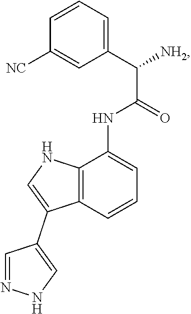 [SMILES: N#Cc1cccc([C@H](N)C(=O)Nc2cccc3c(-c4cn[nH]c4)c[nH]c23)c1]

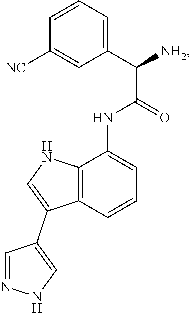 [SMILES: N#Cc1cccc([C@@H](N)C(=O)Nc2cccc3c(-c4cn[nH]c4)c[nH]c23)c1]

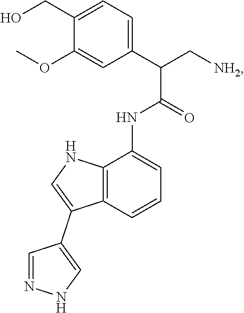 [SMILES: COc1cc(C(CN)C(=O)Nc2cccc3c(-c4cn[nH]c4)c[nH]c23)ccc1CO]

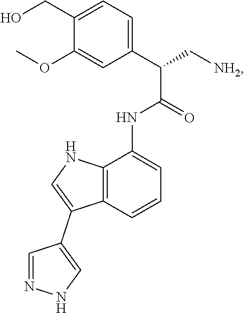 [SMILES: COc1cc([C@H](CN)C(=O)Nc2cccc3c(-c4cn[nH]c4)c[nH]c23)ccc1CO]

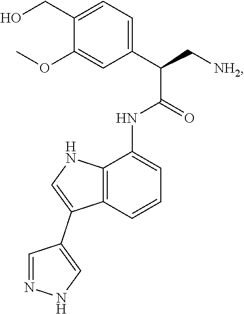 [SMILES: COc1cc([C@@H](CN)C(=O)Nc2cccc3c(-c4cn[nH]c4)c[nH]c23)ccc1CO]

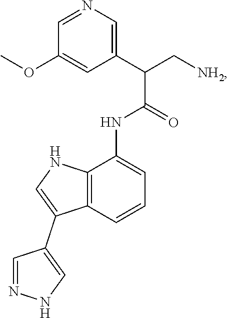 [SMILES: COc1cncc(C(CN)C(=O)Nc2cccc3c(-c4cn[nH]c4)c[nH]c23)c1]

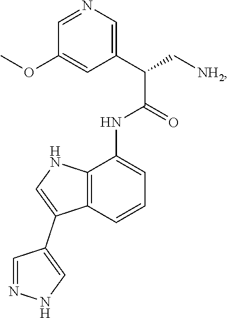 [SMILES: COc1cncc([C@H](CN)C(=O)Nc2cccc3c(-c4cn[nH]c4)c[nH]c23)c1]

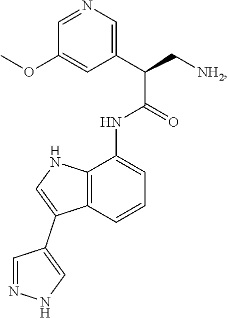 [SMILES: COc1cncc([C@@H](CN)C(=O)Nc2cccc3c(-c4cn[nH]c4)c[nH]c23)c1]

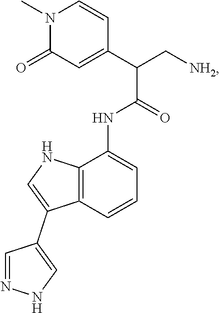 [SMILES: Cn1ccc(C(CN)C(=O)Nc2cccc3c(-c4cn[nH]c4)c[nH]c23)cc1=O]

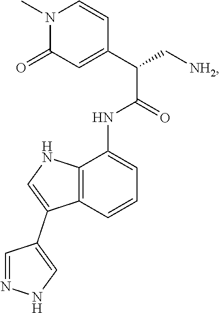 [SMILES: Cn1ccc([C@H](CN)C(=O)Nc2cccc3c(-c4cn[nH]c4)c[nH]c23)cc1=O]

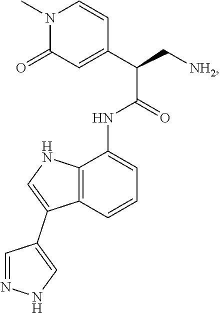 [SMILES: Cn1ccc([C@@H](CN)C(=O)Nc2cccc3c(-c4cn[nH]c4)c[nH]c23)cc1=O]

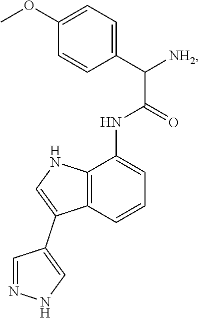 [SMILES: COc1ccc(C(N)C(=O)Nc2cccc3c(-c4cn[nH]c4)c[nH]c23)cc1]

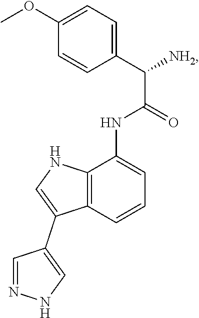 [SMILES: COc1ccc([C@H](N)C(=O)Nc2cccc3c(-c4cn[nH]c4)c[nH]c23)cc1]

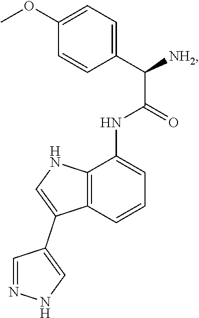 [SMILES: COc1ccc([C@@H](N)C(=O)Nc2cccc3c(-c4cn[nH]c4)c[nH]c23)cc1]

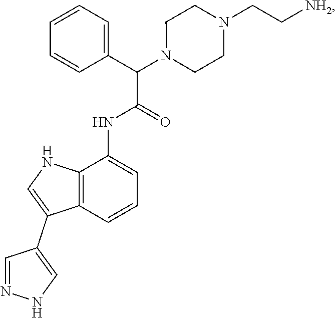 [SMILES: NCCN1CCN(C(C(=O)Nc2cccc3c(-c4cn[nH]c4)c[nH]c23)c2ccccc2)CC1]

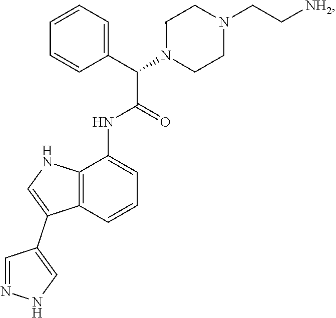 [SMILES: NCCN1CCN([C@H](C(=O)Nc2cccc3c(-c4cn[nH]c4)c[nH]c23)c2ccccc2)CC1]

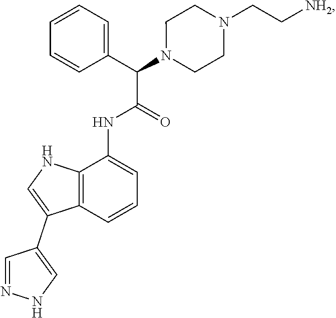 [SMILES: NCCN1CCN([C@@H](C(=O)Nc2cccc3c(-c4cn[nH]c4)c[nH]c23)c2ccccc2)CC1]

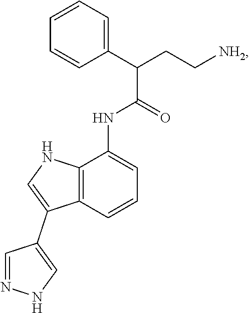 [SMILES: NCCC(C(=O)Nc1cccc2c(-c3cn[nH]c3)c[nH]c12)c1ccccc1]

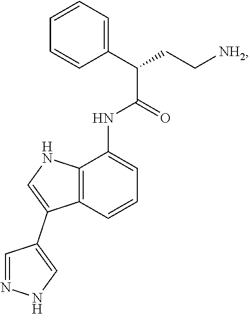 [SMILES: NCC[C@H](C(=O)Nc1cccc2c(-c3cn[nH]c3)c[nH]c12)c1ccccc1]

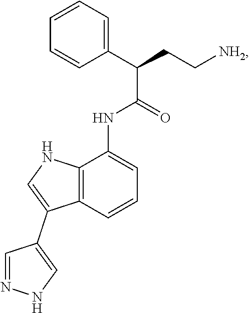 [SMILES: NCC[C@@H](C(=O)Nc1cccc2c(-c3cn[nH]c3)c[nH]c12)c1ccccc1]

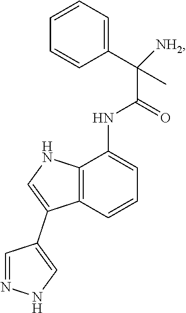 [SMILES: CC(N)(C(=O)Nc1cccc2c(-c3cn[nH]c3)c[nH]c12)c1ccccc1]

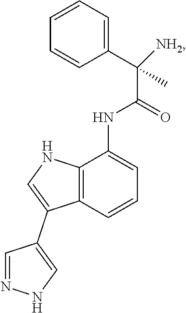 [SMILES: C[C@](N)(C(=O)Nc1cccc2c(-c3cn[nH]c3)c[nH]c12)c1ccccc1]

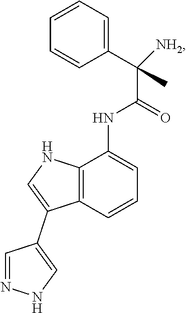 [SMILES: C[C@@](N)(C(=O)Nc1cccc2c(-c3cn[nH]c3)c[nH]c12)c1ccccc1]

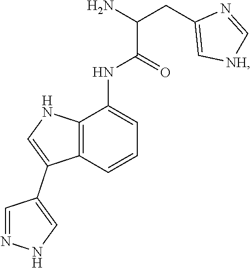 [SMILES: NC(Cc1c[nH]cn1)C(=O)Nc1cccc2c(-c3cn[nH]c3)c[nH]c12]

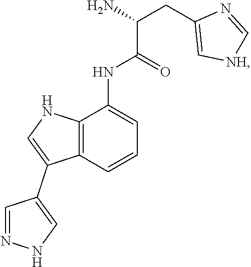 [SMILES: N[C@H](Cc1c[nH]cn1)C(=O)Nc1cccc2c(-c3cn[nH]c3)c[nH]c12]

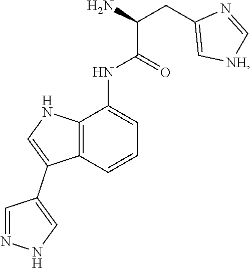 [SMILES: N[C@@H](Cc1c[nH]cn1)C(=O)Nc1cccc2c(-c3cn[nH]c3)c[nH]c12]

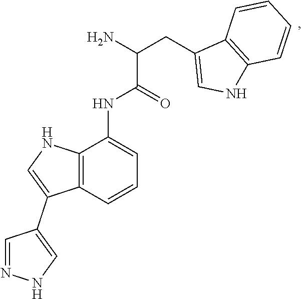 [SMILES: NC(Cc1c[nH]c2ccccc12)C(=O)Nc1cccc2c(-c3cn[nH]c3)c[nH]c12]

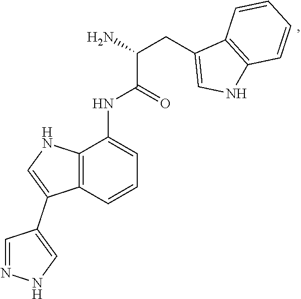 [SMILES: N[C@H](Cc1c[nH]c2ccccc12)C(=O)Nc1cccc2c(-c3cn[nH]c3)c[nH]c12]

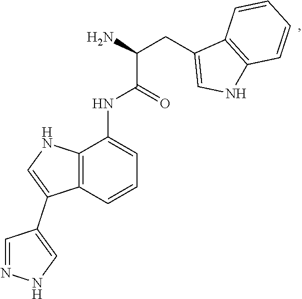 [SMILES: N[C@@H](Cc1c[nH]c2ccccc12)C(=O)Nc1cccc2c(-c3cn[nH]c3)c[nH]c12]

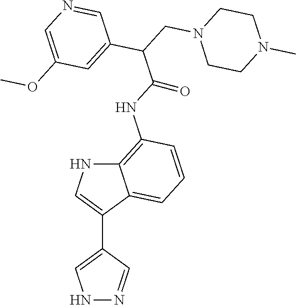

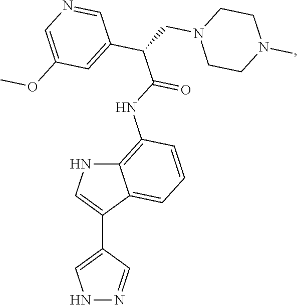

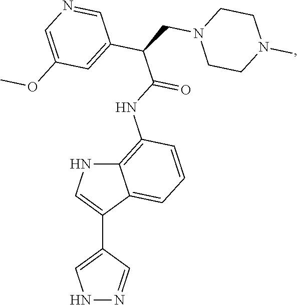

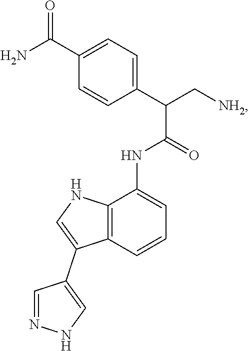 [SMILES: NCC(C(=O)Nc1cccc2c(-c3cn[nH]c3)c[nH]c12)c1ccc(C(N)=O)cc1]

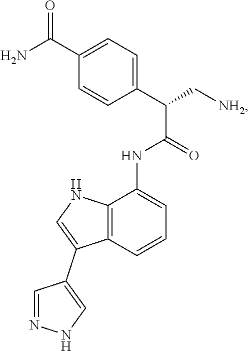 [SMILES: NC[C@H](C(=O)Nc1cccc2c(-c3cn[nH]c3)c[nH]c12)c1ccc(C(N)=O)cc1]

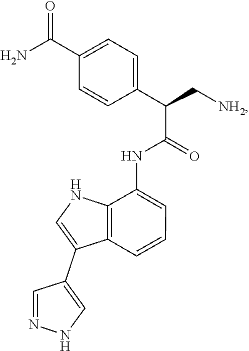 [SMILES: NC[C@@H](C(=O)Nc1cccc2c(-c3cn[nH]c3)c[nH]c12)c1ccc(C(N)=O)cc1]

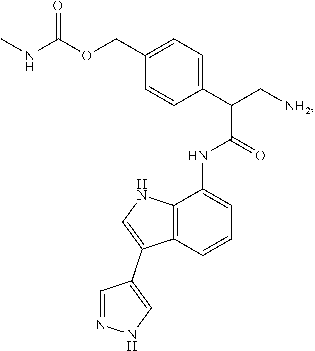 [SMILES: CNC(=O)OCc1ccc(C(CN)C(=O)Nc2cccc3c(-c4cn[nH]c4)c[nH]c23)cc1]

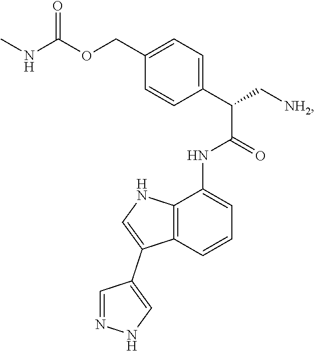 [SMILES: CNC(=O)OCc1ccc([C@H](CN)C(=O)Nc2cccc3c(-c4cn[nH]c4)c[nH]c23)cc1]

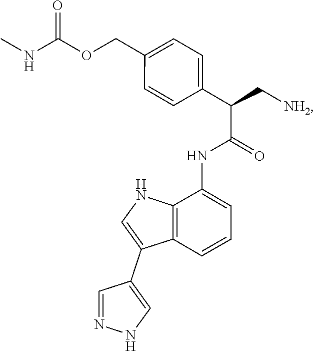 [SMILES: CNC(=O)OCc1ccc([C@@H](CN)C(=O)Nc2cccc3c(-c4cn[nH]c4)c[nH]c23)cc1]

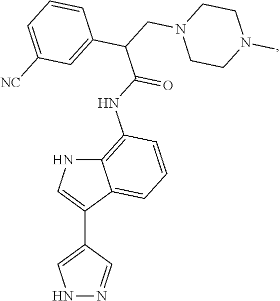

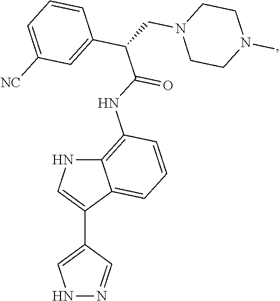

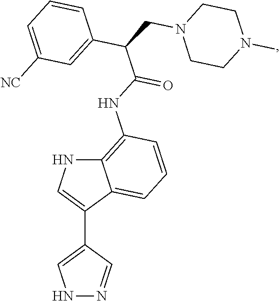

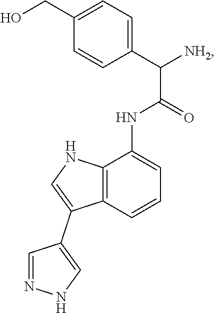 [SMILES: NC(C(=O)Nc1cccc2c(-c3cn[nH]c3)c[nH]c12)c1ccc(CO)cc1]

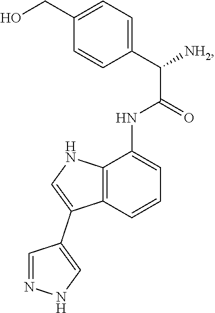 [SMILES: N[C@H](C(=O)Nc1cccc2c(-c3cn[nH]c3)c[nH]c12)c1ccc(CO)cc1]

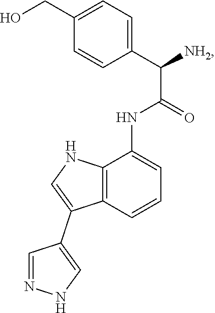 [SMILES: N[C@@H](C(=O)Nc1cccc2c(-c3cn[nH]c3)c[nH]c12)c1ccc(CO)cc1]

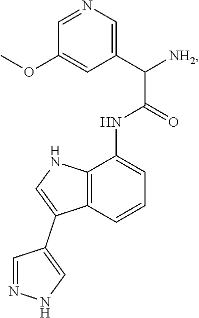 [SMILES: COc1cncc(C(N)C(=O)Nc2cccc3c(-c4cn[nH]c4)c[nH]c23)c1]

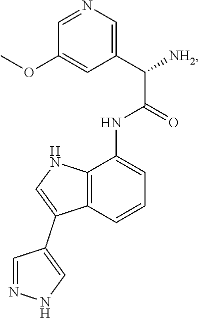 [SMILES: COc1cncc([C@H](N)C(=O)Nc2cccc3c(-c4cn[nH]c4)c[nH]c23)c1]

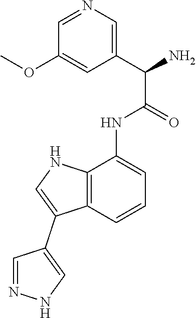 [SMILES: COc1cncc([C@@H](N)C(=O)Nc2cccc3c(-c4cn[nH]c4)c[nH]c23)c1]

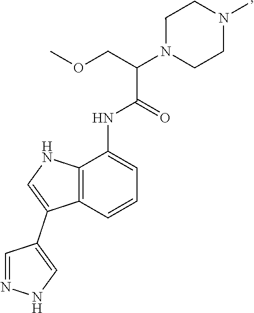

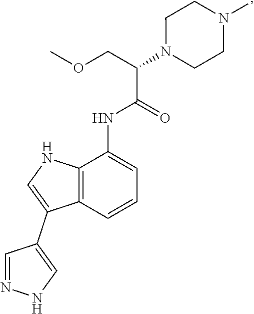

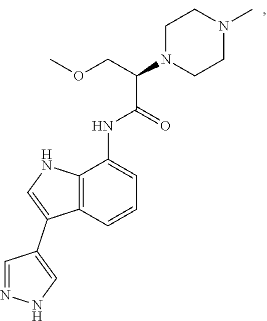 [SMILES: COC[C@H](C(=O)Nc1cccc2c(-c3cn[nH]c3)c[nH]c12)N1CCN(C)CC1]

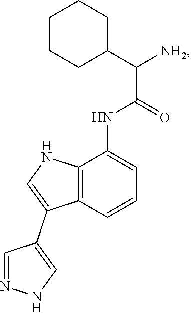 [SMILES: NC(C(=O)Nc1cccc2c(-c3cn[nH]c3)c[nH]c12)C1CCCCC1]

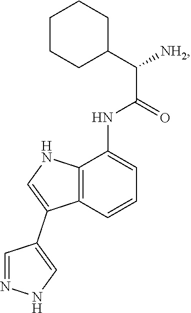 [SMILES: N[C@H](C(=O)Nc1cccc2c(-c3cn[nH]c3)c[nH]c12)C1CCCCC1]

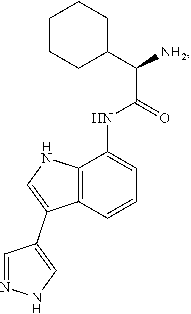 [SMILES: N[C@@H](C(=O)Nc1cccc2c(-c3cn[nH]c3)c[nH]c12)C1CCCCC1]

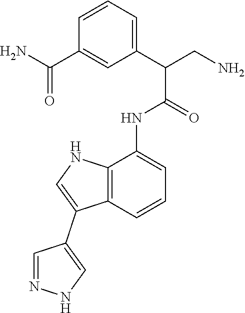 [SMILES: NCC(C(=O)Nc1cccc2c(-c3cn[nH]c3)c[nH]c12)c1cccc(C(N)=O)c1]

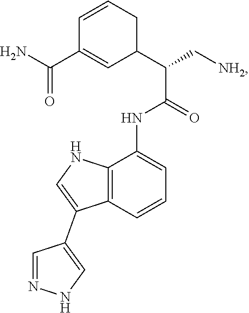 [SMILES: NC[C@H](C(=O)Nc1cccc2c(-c3cn[nH]c3)c[nH]c12)C1C=C(C(N)=O)C=CC1]

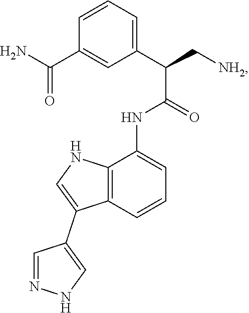 [SMILES: NC[C@@H](C(=O)Nc1cccc2c(-c3cn[nH]c3)c[nH]c12)c1cccc(C(N)=O)c1]

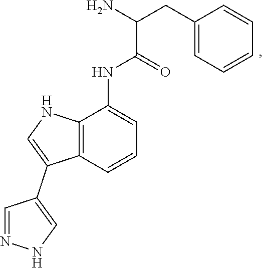 [SMILES: NC(Cc1ccccc1)C(=O)Nc1cccc2c(-c3cn[nH]c3)c[nH]c12]

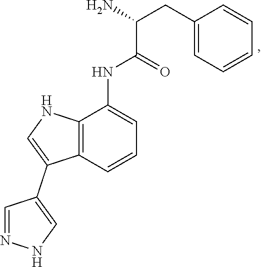 [SMILES: N[C@H](Cc1ccccc1)C(=O)Nc1cccc2c(-c3cn[nH]c3)c[nH]c12]

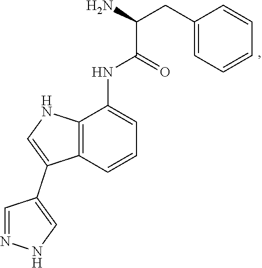 [SMILES: N[C@@H](Cc1ccccc1)C(=O)Nc1cccc2c(-c3cn[nH]c3)c[nH]c12]

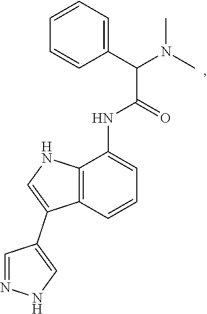 [SMILES: CN(C)C(C(=O)Nc1cccc2c(-c3cn[nH]c3)c[nH]c12)c1ccccc1]

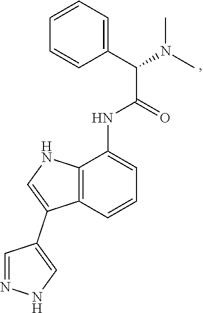 [SMILES: CN(C)[C@H](C(=O)Nc1cccc2c(-c3cn[nH]c3)c[nH]c12)c1ccccc1]

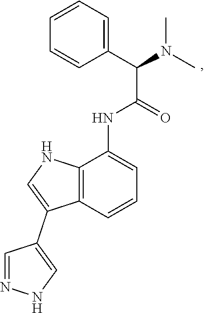 [SMILES: CN(C)[C@@H](C(=O)Nc1cccc2c(-c3cn[nH]c3)c[nH]c12)c1ccccc1]

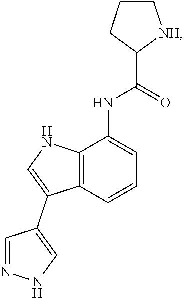 [SMILES: O=C(Nc1cccc2c(-c3cn[nH]c3)c[nH]c12)C1CCCN1]

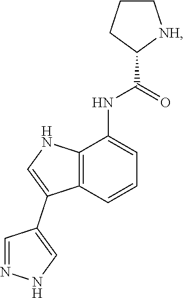 [SMILES: O=C(Nc1cccc2c(-c3cn[nH]c3)c[nH]c12)[C@@H]1CCCN1]

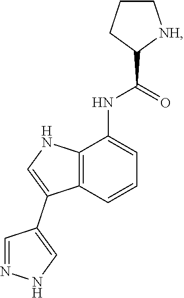

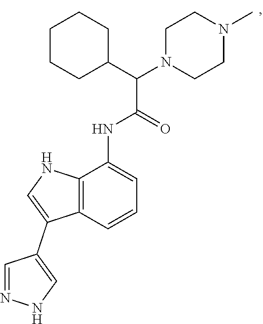 [SMILES: CN1CCN(C(C(=O)Nc2cccc3c(-c4cn[nH]c4)c[nH]c23)C2CCCCC2)CC1]

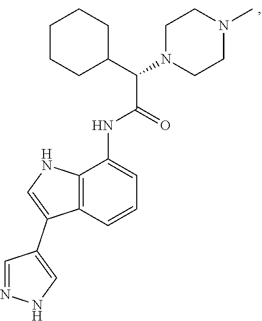 [SMILES: CN1CCN([C@H](C(=O)Nc2cccc3c(-c4cn[nH]c4)c[nH]c23)C2CCCCC2)CC1]

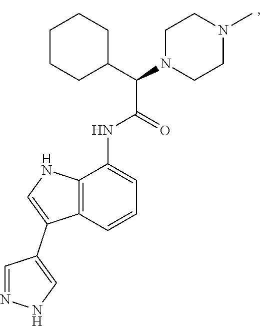 [SMILES: CN1CCN([C@@H](C(=O)Nc2cccc3c(-c4cn[nH]c4)c[nH]c23)C2CCCCC2)CC1]

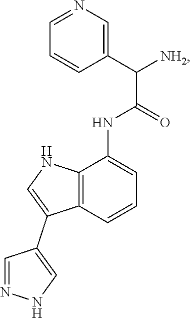 [SMILES: NC(C(=O)Nc1cccc2c(-c3cn[nH]c3)c[nH]c12)c1cccnc1]

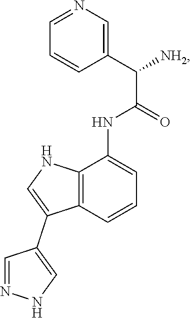 [SMILES: N[C@H](C(=O)Nc1cccc2c(-c3cn[nH]c3)c[nH]c12)c1cccnc1]

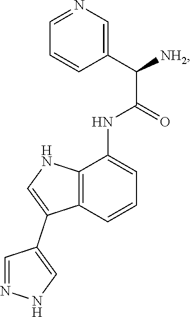 [SMILES: N[C@@H](C(=O)Nc1cccc2c(-c3cn[nH]c3)c[nH]c12)c1cccnc1]

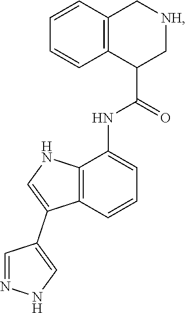 [SMILES: O=C(Nc1cccc2c(-c3cn[nH]c3)c[nH]c12)C1CNCc2ccccc21]

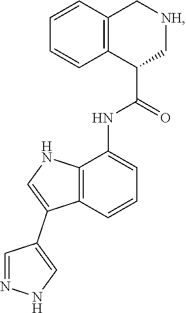 [SMILES: O=C(Nc1cccc2c(-c3cn[nH]c3)c[nH]c12)[C@H]1CNCc2ccccc21]

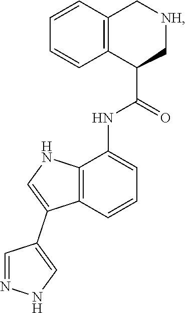 [SMILES: O=C(Nc1cccc2c(-c3cn[nH]c3)c[nH]c12)[C@@H]1CNCc2ccccc21]

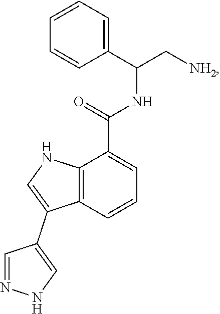 [SMILES: NCC(NC(=O)c1cccc2c(-c3cn[nH]c3)c[nH]c12)c1ccccc1]

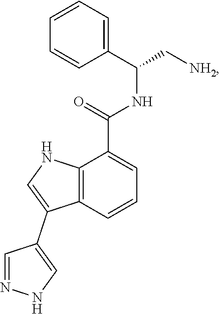 [SMILES: NC[C@H](NC(=O)c1cccc2c(-c3cn[nH]c3)c[nH]c12)c1ccccc1]

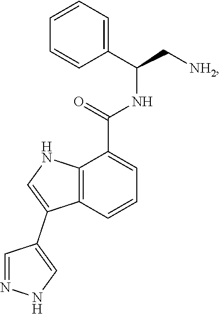 [SMILES: NC[C@@H](NC(=O)c1cccc2c(-c3cn[nH]c3)c[nH]c12)c1ccccc1]

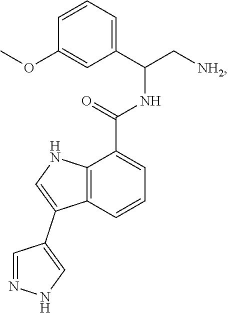 [SMILES: COc1cccc(C(CN)NC(=O)c2cccc3c(-c4cn[nH]c4)c[nH]c23)c1]

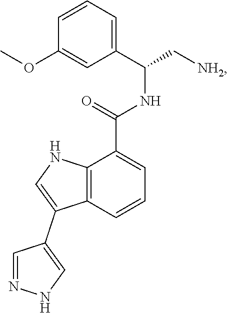 [SMILES: COc1cccc([C@H](CN)NC(=O)c2cccc3c(-c4cn[nH]c4)c[nH]c23)c1]

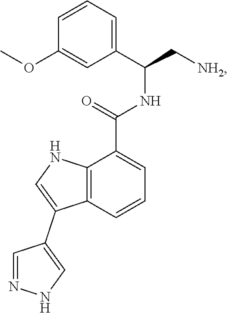 [SMILES: COc1cccc([C@@H](CN)NC(=O)c2cccc3c(-c4cn[nH]c4)c[nH]c23)c1]

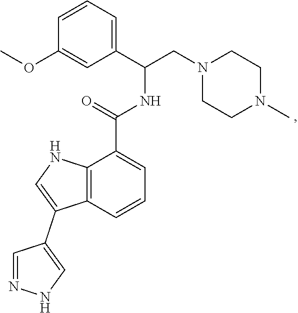

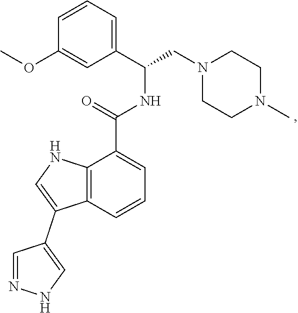

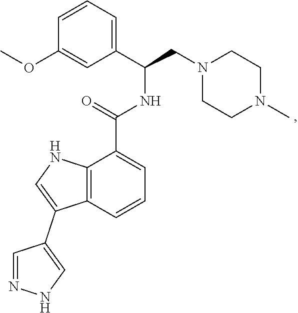

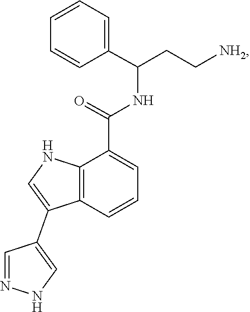 [SMILES: NCCC(NC(=O)c1cccc2c(-c3cn[nH]c3)c[nH]c12)c1ccccc1]

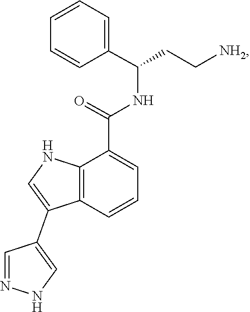 [SMILES: NCC[C@H](NC(=O)c1cccc2c(-c3cn[nH]c3)c[nH]c12)c1ccccc1]

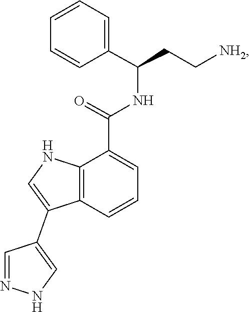 [SMILES: NCC[C@@H](NC(=O)c1cccc2c(-c3cn[nH]c3)c[nH]c12)c1ccccc1]

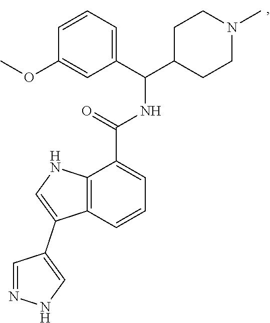

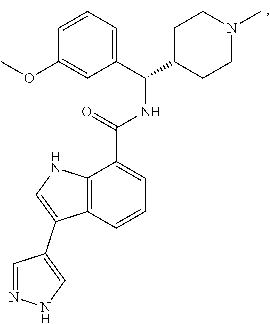

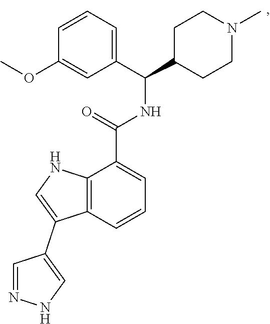

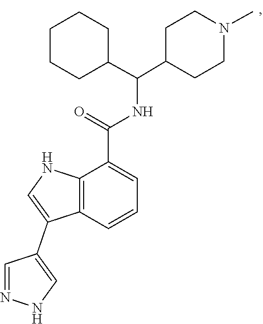 [SMILES: CN1CCC(C(NC(=O)c2cccc3c(-c4cn[nH]c4)c[nH]c23)C2CCCCC2)CC1]

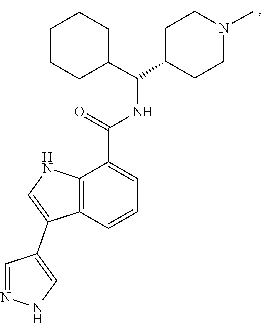

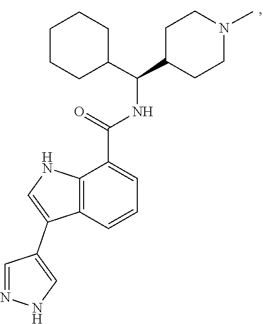 [SMILES: CN1CCC([C@@H](NC(=O)c2cccc3c(-c4cn[nH]c4)c[nH]c23)C2CCCCC2)CC1]

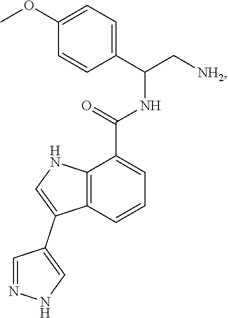 [SMILES: COc1ccc(C(CN)NC(=O)c2cccc3c(-c4cn[nH]c4)c[nH]c23)cc1]

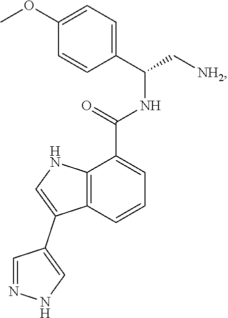 [SMILES: COc1ccc([C@H](CN)NC(=O)c2cccc3c(-c4cn[nH]c4)c[nH]c23)cc1]

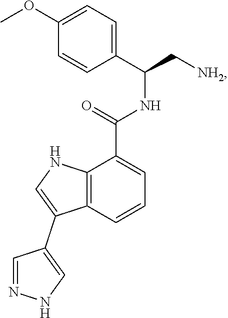 [SMILES: COc1ccc([C@@H](CN)NC(=O)c2cccc3c(-c4cn[nH]c4)c[nH]c23)cc1]

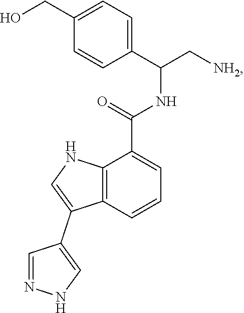 [SMILES: NCC(NC(=O)c1cccc2c(-c3cn[nH]c3)c[nH]c12)c1ccc(CO)cc1]

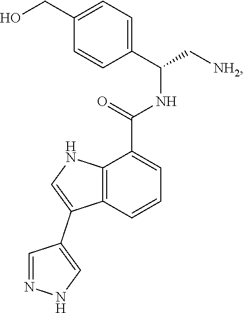 [SMILES: NC[C@H](NC(=O)c1cccc2c(-c3cn[nH]c3)c[nH]c12)c1ccc(CO)cc1]

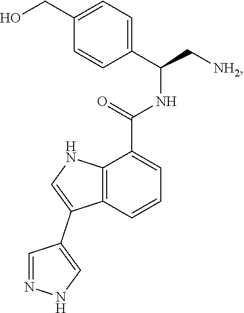 [SMILES: NC[C@@H](NC(=O)c1cccc2c(-c3cn[nH]c3)c[nH]c12)c1ccc(CO)cc1]

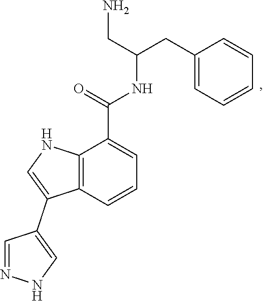 [SMILES: NCC(Cc1ccccc1)NC(=O)c1cccc2c(-c3cn[nH]c3)c[nH]c12]

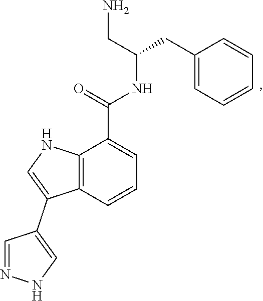 [SMILES: NC[C@H](Cc1ccccc1)NC(=O)c1cccc2c(-c3cn[nH]c3)c[nH]c12]

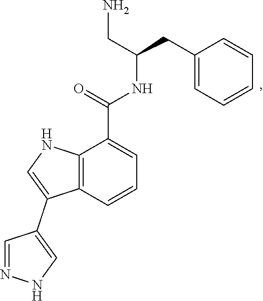 [SMILES: NC[C@@H](Cc1ccccc1)NC(=O)c1cccc2c(-c3cn[nH]c3)c[nH]c12]

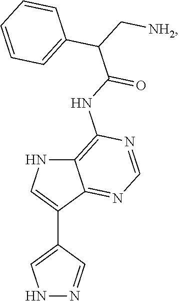 [SMILES: NCC(C(=O)Nc1ncnc2c(-c3cn[nH]c3)c[nH]c12)c1ccccc1]

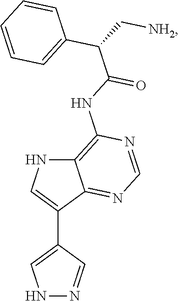 [SMILES: NC[C@H](C(=O)Nc1ncnc2c(-c3cn[nH]c3)c[nH]c12)c1ccccc1]

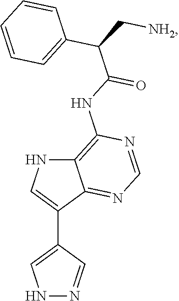 [SMILES: NC[C@@H](C(=O)Nc1ncnc2c(-c3cn[nH]c3)c[nH]c12)c1ccccc1]

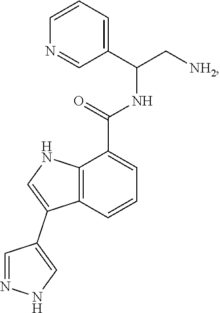 [SMILES: NCC(NC(=O)c1cccc2c(-c3cn[nH]c3)c[nH]c12)c1cccnc1]

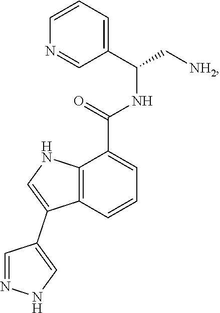 [SMILES: NC[C@H](NC(=O)c1cccc2c(-c3cn[nH]c3)c[nH]c12)c1cccnc1]

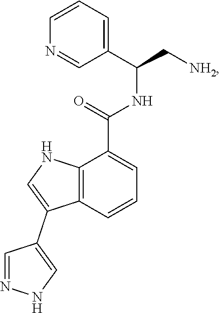 [SMILES: NC[C@@H](NC(=O)c1cccc2c(-c3cn[nH]c3)c[nH]c12)c1cccnc1]

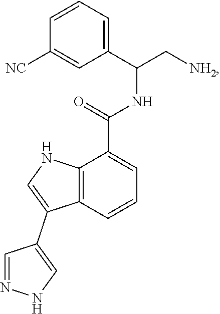 [SMILES: N#Cc1cccc(C(CN)NC(=O)c2cccc3c(-c4cn[nH]c4)c[nH]c23)c1]

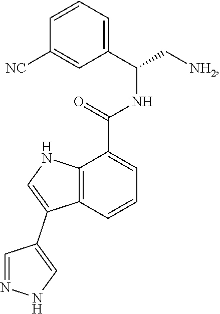 [SMILES: N#Cc1cccc([C@H](CN)NC(=O)c2cccc3c(-c4cn[nH]c4)c[nH]c23)c1]

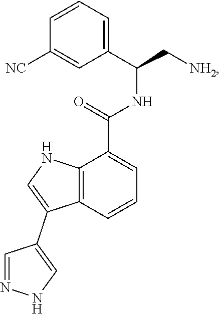 [SMILES: N#Cc1cccc([C@@H](CN)NC(=O)c2cccc3c(-c4cn[nH]c4)c[nH]c23)c1]

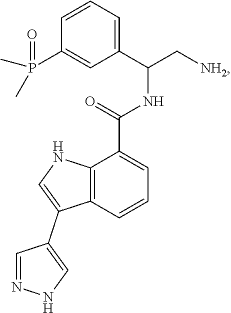 [SMILES: CP(C)(=O)c1cccc(C(CN)NC(=O)c2cccc3c(-c4cn[nH]c4)c[nH]c23)c1]

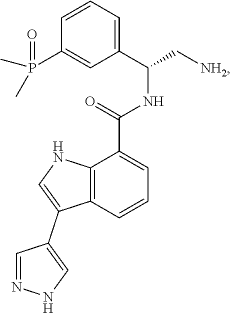 [SMILES: CP(C)(=O)c1cccc([C@H](CN)NC(=O)c2cccc3c(-c4cn[nH]c4)c[nH]c23)c1]

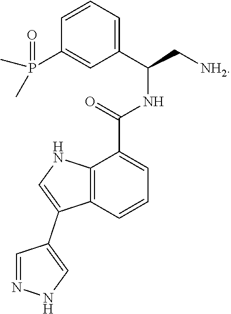 [SMILES: CP(C)(=O)c1cccc([C@@H](CN)NC(=O)c2cccc3c(-c4cn[nH]c4)c[nH]c23)c1]

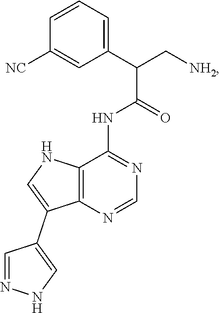 [SMILES: N#Cc1cccc(C(CN)C(=O)Nc2ncnc3c(-c4cn[nH]c4)c[nH]c23)c1]

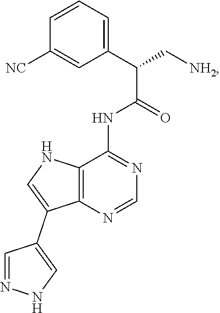 [SMILES: N#Cc1cccc([C@H](CN)C(=O)Nc2ncnc3c(-c4cn[nH]c4)c[nH]c23)c1]

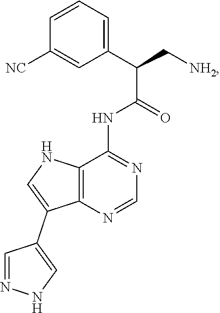 [SMILES: N#Cc1cccc([C@@H](CN)C(=O)Nc2ncnc3c(-c4cn[nH]c4)c[nH]c23)c1]

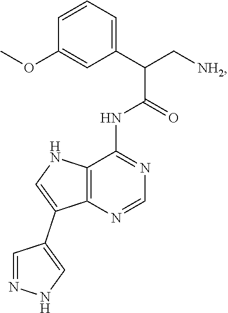 [SMILES: COc1cccc(C(CN)C(=O)Nc2ncnc3c(-c4cn[nH]c4)c[nH]c23)c1]

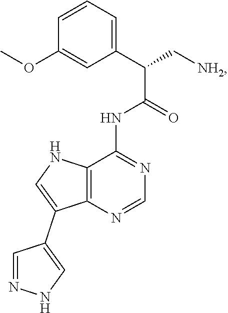 [SMILES: COc1cccc([C@H](CN)C(=O)Nc2ncnc3c(-c4cn[nH]c4)c[nH]c23)c1]

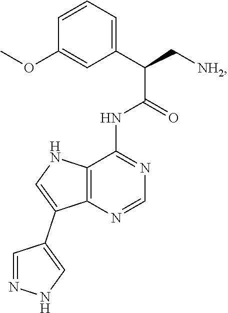 [SMILES: COc1cccc([C@@H](CN)C(=O)Nc2ncnc3c(-c4cn[nH]c4)c[nH]c23)c1]

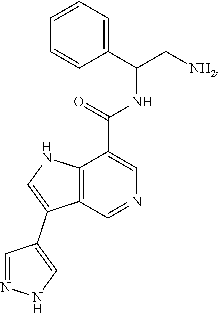 [SMILES: NCC(NC(=O)c1cncc2c(-c3cn[nH]c3)c[nH]c12)c1ccccc1]

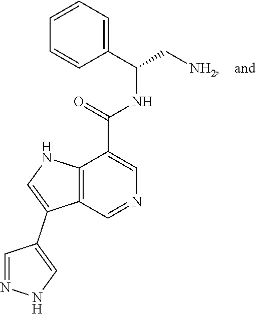 [SMILES: NC[C@H](NC(=O)c1cncc2c(-c3cn[nH]c3)c[nH]c12)c1ccccc1 ; "and"]

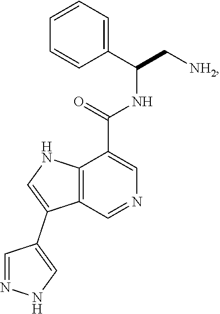 [SMILES: NC[C@@H](NC(=O)c1cncc2c(-c3cn[nH]c3)c[nH]c12)c1ccccc1]

Compounds provided herein are described with reference to both generic formulae and specific compounds. In addition, the compounds of the present disclosure may exist in a number of different forms or derivatives, including but not limited to prodrugs, active metabolic derivatives (active metabolites), solvates, pharmaceutically acceptable salts or isotope derivatives, all of which are within the scope of the present disclosure.

As used herein, the term “prodrugs” refers to compounds or pharmaceutically acceptable salts thereof which, when metabolized under physiological conditions or when converted by solvolysis, yield the desired active compound. Prodrugs include, without limitation, esters, amides, carbamates, carbonates, ureides, solvates, or hydrates of the active compound. Typically, the prodrug is inactive, or less active than the active compound, but may provide one or more advantageous handling, administration, and/or metabolic properties. For example, some prodrugs are esters of the active compound; during metabolysis, the ester group is cleaved to yield the active drug. Also, some prodrugs are activated enzymatically to yield the active compound, or a compound which, upon further chemical reaction, yields the active compound. Prodrugs may proceed from prodrug form to active form in a single step or may have one or more intermediate forms which may themselves have activity or may be inactive. Preparation and use of prodrugs is discussed in T. Higuchi and V. Stella, “Pro-drugs as Novel Delivery Systems”, Vol. 14 of the A.C.S. Symposium Series, in Bioreversible Carriers in Drug Design, ed. Edward B. Roche, American Pharmaceutical Association and Pergamon Press, 1987; in Prodrugs: Challenges and Rewards, ed. V. Stella, R. Borchardt, M. Hageman, R. Oliyai, H. Maag, J. Tilley, Springer-Verlag New York, 2007, all of which are hereby incorporated by reference in their entirety.

As used herein, the term “metabolite”, e.g., active metabolite overlaps with prodrug as described above. Thus, such metabolites are pharmacologically active compounds or compounds that further metabolize to pharmacologically active compounds that are derivatives resulting from metabolic process in the body of a subject. For example, such metabolites may result from oxidation, reduction, hydrolysis, amidation, deamidation, esterification, deesterification, enzymatic cleavage, and the like, of the administered compound or salt or prodrug. Of these, active metabolites are such pharmacologically active derivative compounds. For prodrugs, the prodrug compound is generally inactive or of lower activity than the metabolic product. For active metabolites, the parent compound may be either an active compound or may be an inactive prodrug.

Prodrugs and active metabolites may be identified using routine techniques know in the art. See, e.g., Bertolini et al, 1997, J Med Chem 40:2011-2016; Shan et al., J Pharm Sci 86:756-757; Bagshawe, 1995, DrugDev Res 34:220-230; Wermuth, supra.

As used herein, the term “pharmaceutically acceptable” indicates that the substance or composition is compatible chemically and/or toxicologically, with the other ingredients comprising a formulation, and/or the subjects being treated therewith.

As used herein, the term “pharmaceutically acceptable salt”, unless otherwise indicated, includes salts that retain the biological effectiveness of the free acids and bases of the specified compound and that are not biologically or otherwise undesirable. Contemplated pharmaceutically acceptable salt forms include, but are not limited to, mono, bis, tris, tetrakis, and so on. Pharmaceutically acceptable salts are non-toxic in the amounts and concentrations at which they are administered. The preparation of such salts can facilitate the pharmacological use by altering the physical characteristics of a compound without preventing it from exerting its physiological effect. Useful alterations in physical properties include lowering the melting point to facilitate transmucosal administration and increasing the solubility to facilitate administering higher concentrations of the drug.

Pharmaceutically acceptable salts include acid addition salts such as those containing sulfate, chloride, hydrochloride, fumarate, maleate, phosphate, sulfamate, acetate, citrate, lactate, tartrate, methanesulfonate, ethanesulfonate, benzenesulfonate, p-toluenesulfonate, cyclohexylsulfamate and quinate. Pharmaceutically acceptable salts can be obtained from acids such as hydrochloric acid, maleic acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, fumaric acid, and quinic acid.

Pharmaceutically acceptable salts also include basic addition salts such as those containing benzathine, chloroprocaine, choline, diethanolamine, ethanolamine, t-butylamine, ethylenediamine, meglumine, procaine, aluminum, calcium, lithium, magnesium, potassium, sodium, ammonium, alkylamine, and zinc, when acidic functional groups, such as carboxylic acid or phenol are present. For example, see Remington's Pharmaceutical Sciences, 19^{th} ed., Mack Publishing Co., Easton, PA, Vol. 2, p. 1457, 1995; “Handbook of Pharmaceutical Salts: Properties, Selection, and Use” by Stahl and Wermuth, Wiley-VCH, Weinheim, Germany, 2002. Such salts can be prepared using the appropriate corresponding bases.

Pharmaceutically acceptable salts can be prepared by standard techniques. For example, the free-base form of a compound can be dissolved in a suitable solvent, such as an aqueous or aqueous-alcohol solution containing the appropriate acid and then isolated by evaporating the solution. Thus, if the particular compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

Similarly, if the particular compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as L-glycine, L-lysine, and L-arginine, ammonia, primary, secondary, and tertiary amines, and cyclic amines, such as hydroxyethylpyrrolidine, piperidine, morpholine or piperazine, and inorganic salts derived from sodium, calcium, potassium, magnesium, manganese, iron, copper, zinc, aluminum and lithium.

It is also to be understood that the compounds of present disclosure can exist in unsolvated forms, solvated forms (e.g., hydrated forms), and solid forms (e.g., crystal or polymorphic forms), and the present disclosure is intended to encompass all such forms.

As used herein, the term “solvate” or “solvated form” refers to solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O. Examples of solvents that form solvates include, but are not limited to, water, isopropanol, ethanol, methanol, DMSO, ethyl acetate, acetic acid, and ethanolamine.

The present disclosure is also intended to include all isotopes of atoms in the compounds. Isotopes of an atom include atoms having the same atomic number but different mass numbers. For example, unless otherwise specified, hydrogen, carbon, nitrogen, oxygen, phosphorous, sulfur, fluorine, chlorine, bromide or iodine in the compounds of present disclosure are meant to also include their isotopes, such as but not limited to ¹H, ²H, ³H, ¹¹C, ¹²C, ¹³C, ¹⁴C, ¹⁴N, ¹⁵N, ¹⁶O, ¹⁷O, ¹⁸O, ³¹P, ³²P, ³²S, ³³S, ³⁴S, ³⁶S, ¹⁷F, ¹⁸F, ¹⁹F, ³⁵Cl, ³⁷Cl, ⁷⁹Br, ⁸¹Br, ¹²⁴I, ¹²⁷I and ¹³¹I. In some embodiments, hydrogen includes protium, deuterium and tritium. In some embodiments, carbon includes ¹²C and ¹³C.

Compounds provided herein or pharmaceutically acceptable salts thereof may contain one or more asymmetric centers and thus give rise to enantiomers, diastereomers, and other stereoisomeric forms that may be defined, in terms of absolute stereochemistry, as (R)- or (S)- or, as (D)- or (L)- for amino acids, or in terms of relative configuration, as rel-(R)- or rel-(S)-. The present disclosure includes all such possible isomers, as well as their racemic and optically pure forms. Optically active (+) and (−), (R)- and (S)-, or (D)- and (L)-isomers may be prepared using chiral synthons or chiral reagents, or resolved by conventional techniques, such as, chromatography and fractional crystallization. Traditional techniques for the preparation, isolation of individual enantiomers include chiral synthesis from a suitable optically pure precursor or resolution of the racemate (or the racemate of a salt or derivative) using, for example, chiral high pressure liquid chromatography (HPLC). Wherever compounds are represented in their chiral form, it is understood that the embodiment includes, but is not limited to, the specific diastereomerically or enantiomerically enriched form. In situations that the chirality is not specified but is present, it is understood that the embodiment is intended to include either the specific diastereomerically or enantiomerically enriched form; or a racemic or scalemic mixture of such compound(s).

The term “stereoisomer” refers to a compound containing the same atoms bonded by the same bonds but having different three-dimensional structures, which are not interchangeable. The present disclosure contemplates various stereoisomers and mixtures thereof and includes “enantiomers”, which refers to two stereoisomers whose molecules are non-superimposable mirror images of one another.

The term “enantiomers” represent a pair of stereoisomers that are non-superimposable mirror images of each other. A 1:1 mixture of a pair of enantiomers is a “racemic” mixture. A mixture of enantiomers at a ratio other than 1:1 is a “scalemic” mixture.

The term “diastereoisomers” represent stereoisomers that have at least two asymmetric atoms, but which are not mirror-images of each other.

The term “tautomer” or “tautomeric form” refers to structural isomers of different energies which are interconvertible via a low energy barrier. The presence and concentrations of the isomeric forms will depend on the environment the compound is found in and may be different depending upon, for example, whether the compound is a solid or is in an organic or aqueous solution. By way of examples, proton tautomers (also known as prototropic tautomers) include interconversions via migration of a proton, such as keto-enol, amide-imidic acid, lactam-lactim, imine-enamine isomerizations and annular forms where a proton can occupy two or more positions of a heterocyclic system. Valence tautomers include interconversions by reorganization of some of the bonding electrons. Tautomers can be in equilibrium or sterically locked into one form by appropriate substitution. Compounds of the present disclosure identified by name or structure as one particular tautomeric form are intended to include other tautomeric forms unless otherwise specified.

When the compounds provided herein contain olefinic double bonds or other centers of geometric asymmetry, and unless specified otherwise, these compounds include both E and Z geometric isomers.

### Synthetic Method

The compounds provided herein can be prepared using any known organic synthesis techniques and can be synthesized according to any of numerous possible synthetic routes

Reactions for preparing compounds of the present disclosure can be carried out in suitable solvents, which can be readily selected by one skilled in the art of organic synthesis. Suitable solvents can be substantially non-reactive with starting materials (reactants), intermediates, or products at the temperatures at which the reactions are carried out, e.g. temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected by one skilled in the art.

Preparation of compounds of the present disclosure can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd Ed., Wiley & Sons, Inc., New York (1999), in P. Kocienski, Protecting Groups, Georg Thieme Verlag, 2003, and in Peter G. M. Wuts, Greene's Protective Groups in Organic Synthesis, 5^{th} Edition, Wiley, 2014, all of which are incorporated herein by reference in its entirety.

Reactions can be monitored according to any suitable method known in the art. For example, product formation can be monitored by spectroscopic means, such as nuclear magnetic resonance spectroscopy (e.g. ¹H or ¹³C), infrared spectroscopy, spectrophotometry (e.g. UV-visible), mass spectrometry, or by chromatographic methods such as high performance liquid chromatography (HPLC), liquid chromatography-mass spectroscopy (LCMS), or thin layer chromatography (TLC). Compounds can be purified by one skilled in the art by a variety of methods, including high performance liquid chromatography (HPLC) (“Preparative LC-MS Purification: Improved Compound Specific Method Optimization” Karl F. Blom, Brian Glass, Richard Sparks, Andrew P. Combs J. Combi. Chem. 2004, 6 (6), 874-883, which is incorporated herein by reference in its entirety), and normal phase silica chromatography.

### Pharmaceutical Composition

In a further aspect, there is provided pharmaceutical compositions comprising one or more compounds of the present disclosure, or a pharmaceutically acceptable salt thereof. In some embodiments, the pharmaceutical compositions of the present disclosure comprise a first compound of Formula (I) or a pharmaceutically acceptable salt thereof and one or more additional compounds of the same formula but said first compound and additional compounds are not the same molecules.

In another aspect, there is provided pharmaceutical composition comprising one or more compounds of the present disclosure, or a pharmaceutically acceptable salt thereof, and at least one pharmaceutical acceptable excipient.

In some embodiments, the pharmaceutical compositions of the present disclosure comprises a therapeutically effective amount of one or more compounds of the present disclosure or a pharmaceutically acceptable salt thereof.

In some embodiments, the pharmaceutical compositions of the present disclosure comprises a therapeutically effective amount of one or more compounds of the present disclosure or a pharmaceutically acceptable salt thereof, and at least one pharmaceutical acceptable excipient.

As used herein, the term “therapeutically effective amount” refers to an amount of a molecule, compound, or composition comprising the molecule or compound to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; the rate of administration; the therapeutic or combination of therapeutics selected for administration; and the discretion of the prescribing physician. Therapeutically effective amounts for a given situation can be determined by routine experimentation that is within the skill and judgment of the clinician.

As used herein, the term “pharmaceutical composition” refers to a formulation containing the molecules or compounds of the present disclosure in a form suitable for administration to a subject. The pharmaceutical compositions include compositions suitable adapted for oral administration, rectal administration, topical administration, parenteral (including subcutaneous, intramuscular, and intravenous) administration, sublingual administration, ocular administration, transdermal administration or nasal administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used herein includes both one and more than one such excipient. The term “pharmaceutically acceptable excipient” also encompasses “pharmaceutically acceptable carrier” and “pharmaceutically acceptable diluent”.

The particular excipient used will depend upon the means and purpose for which the compounds of the present disclosure are being applied. Solvents are generally selected based on solvents recognized by persons skilled in the art as safe to be administered to a mammal including humans. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof.

In some embodiments, suitable excipients may include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In some embodiments, suitable excipients may include one or more stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present disclosure or pharmaceutical composition thereof) or aid in the manufacturing of the pharmaceutical product (i.e., medicament). The active pharmaceutical ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980). A “liposome” is a small vesicle composed of various types of lipids, phospholipids and/or surfactant which is useful for delivery of a drug (such as the compounds disclosed herein and, optionally, a chemotherapeutic agent) to a mammal including humans. The components of the liposome are commonly arranged in a bilayer formation, similar to the lipid arrangement of biological membranes.

The pharmaceutical compositions provided herein can be in any form that allows for the composition to be administered to a subject, including, but not limited to a human, and formulated to be compatible with an intended route of administration.

A variety of routes are contemplated for the pharmaceutical compositions provided herein, and accordingly the pharmaceutical composition provided herein may be supplied in bulk or in unit dosage form depending on the intended administration route. For example, for oral, buccal, and sublingual administration, powders, suspensions, granules, tablets, pills, capsules, gelcaps, and caplets may be acceptable as solid dosage forms, and emulsions, syrups, elixirs, suspensions, and solutions may be acceptable as liquid dosage forms. For injection administration, emulsions and suspensions may be acceptable as liquid dosage forms, and a powder suitable for reconstitution with an appropriate solution as solid dosage forms. For inhalation administration, solutions, sprays, dry powders, and aerosols may be acceptable dosage form. For topical (including buccal and sublingual) or transdermal administration, powders, sprays, ointments, pastes, creams, lotions, gels, solutions, and patches may be acceptable dosage form. For vaginal administration, pessaries, tampons, creams, gels, pastes, foams and spray may be acceptable dosage form.

In some embodiments, the pharmaceutical compositions of the present disclosure may be in a form of formulation for oral administration.

In certain embodiments, the pharmaceutical compositions of the present disclosure may be in the form of tablet formulations. Suitable pharmaceutically-acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case using conventional coating agents and procedures well known in the art.

In certain embodiments, the pharmaceutical compositions of the present disclosure may be in a form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

In certain embodiments, the pharmaceutical compositions of the present disclosure may be in the form of aqueous suspensions, which generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), coloring agents, flavoring agents, and/or sweetening agents (such as sucrose, saccharine or aspartame).

In certain embodiments, the pharmaceutical compositions of the present disclosure may be in the form of oily suspensions, which generally contain suspended active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

In certain embodiments, the pharmaceutical compositions of the present disclosure may be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

In certain embodiments, the pharmaceutical compositions provided herein may be in the form of syrups and elixirs, which may contain sweetening agents such as glycerol, propylene glycol, sorbitol, aspartame or sucrose, a demulcent, a preservative, a flavoring and/or coloring agent.

In some embodiments, the pharmaceutical compositions of the present disclosure may be in a form of formulation for injection administration.

In certain embodiments, the pharmaceutical compositions of the present disclosure may be in the form of a sterile injectable preparation, such as a sterile injectable aqueous or oleaginous suspension. This suspension may be formulated according to the known art using those suitable dispersing or wetting agents and suspending agents, which have been mentioned above. The sterile injectable preparation may also be a sterile injectable solution or suspension in a non-toxic parenterally acceptable diluent or solvent, such as a solution in 1,3-butanediol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose, any bland fixed oil may be employed including synthetic mono- or diglycerides. In addition, fatty acids such as oleic acid may likewise be used in the preparation of injectables.

In some embodiments, the pharmaceutical compositions of the present disclosure may be in a form of formulation for inhalation administration.

In certain embodiments, the pharmaceutical compositions of the present disclosure may be in the form of aqueous and nonaqueous (e.g., in a fluorocarbon propellant) aerosols containing any appropriate solvents and optionally other compounds such as, but not limited to, stabilizers, antimicrobial agents, antioxidants, pH modifiers, surfactants, bioavailability modifiers and combinations of these. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols.

In some embodiments, the pharmaceutical compositions of the present disclosure may be in a form of formulation for topical or transdermal administration. In certain embodiments, the pharmaceutical compositions provided herein may be in the form of creams, ointments, gels and aqueous or oily solutions or suspensions, which may generally be obtained by formulating an active ingredient with a conventional, topically acceptable excipients such as animal and vegetable fats, oils, waxes, paraffins, starch, tragacanth, cellulose derivatives, polyethylene glycols, silicones, bentonites, silicic acid, talc and zinc oxide, or mixtures thereof.

In certain embodiments, the pharmaceutical compositions provided herein may be formulated for administration ocularly. In certain embodiments, the pharmaceutical compostions provided herein may be in the form of ophthalmic formulation, such as eye ointments, powders, solutions and the like. In certain embodiments, ophthalmic formulations are prepared at a comfortable pH with an appropriate buffer system.

Besides those representative dosage forms described above, pharmaceutically acceptable excipients and carriers are generally known to those skilled in the art and are thus included in the present disclosure. Such excipients and carriers are described, for example, in “Remingtons Pharmaceutical Sciences” Mack Pub. Co., New Jersey (1991), in “Remington: The Science and Practice of Pharmacy”, Ed. University of the Sciences in Philadelphia, 21^{st} Edition, LWW (2005), which are incorporated herein by reference.

The dosage regimen for the compounds provided herein will vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. A physician or veterinarian can determine and prescribe the effective amount of the drug required to prevent, counter, or arrest the progress of the disorder.

In some embodiments, the pharmaceutical compositions of the present disclosure can be formulated so that a dosage of between 0.001-1000 mg/kg body weight/day, for example, 0.01-800 mg/kg body weight/day, 0.01-700 mg/kg body weight/day, 0.01-600 mg/kg body weight/day, 0.01-500 mg/kg body weight/day, 0.01-400 mg/kg body weight/day, 0.01-300 mg/kg body weight/day, 0.1-200 mg/kg body weight/day, 0.1-150 mg/kg body weight/day, 0.1-100 mg/kg body weight/day, 0.5-100 mg/kg body weight/day, 0.5-80 mg/kg body weight/day, 0.5-60 mg/kg body weight/day, 0.5-50 mg/kg body weight/day, 1-50 mg/kg body weight/day, 1-45 mg/kg body weight/day, 1-40 mg/kg body weight/day, 1-35 mg/kg body weight/day, 1-30 mg/kg body weight/day, 1-25 mg/kg body weight/day of the compounds provided herein, or a pharmaceutically acceptable salt thereof, can be administered. In some instances, dosage levels below the lower limit of the aforesaid range may be more than adequate, while in other cases still larger doses may be employed without causing any harmful side effect, provided that such larger doses are first divided into several small doses for administration throughout the day. For further information on routes of administration and dosage regimes, see Chapter 25.3 in Volume 5 of Comprehensive Medicinal Chemistry (Corwin Hansch; Chairman of Editorial Board), Pergamon Press 1990, which is specifically incorporated herein by reference.

In some embodiments, the pharmaceutical compositions of the present disclosure can be formulated as a single dosage form. The amount of the compounds provided herein in the single dosage form will vary depending on the subject treated and particular mode of administration.

In some embodiments, dosage forms suitable for administration may contain from about 1 mg to about 1000 mg of active ingredient per dosage unit. In these pharmaceutical compositions the active ingredient will ordinarily be present in an amount of about 0.1-95% by weight based on the total weight of the composition.

In some embodiments, the pharmaceutical compositions of the present disclosure can be formulated as short-acting, fast-releasing, long-acting, and sustained-releasing. Accordingly, the pharmaceutical formulations of the present disclosure may also be formulated for controlled release or for slow release.

In some embodiments, a dose of the compounds provided herein or the pharmaceutical compositions provided herein is administered to a subject every day, every other day, every couple of days, every third day, once a week, twice a week, three times a week, or once every two weeks. If desired, the effective daily dose of the active compound may be administered as two, three, four, five, six or more sub-doses administered separately at appropriate intervals throughout the day, optionally, in unit dosage forms. In some embodiments, a dose of the compounds provided herein or the pharmaceutical compositions provided herein is administered for 2 days, 3 days, 5 days, 7 days, 14 days, 21 days, 1 month, 2 months, 2.5 months, 3 months, 4 months, 5 months, 6 months or more.

In a further aspect, there is also provided veterinary compositions comprising one or more molecules or compounds of the present disclosure or pharmaceutically acceptable salts thereof and a veterinary carrier. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

The pharmaceutical compositions or veterinary compositions may be packaged in a variety of ways depending upon the method used for administering the drug. For example, an article for distribution can include a container having deposited therein the compositions in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings. The compositions may also be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described.

In some embodiments, the pharmaceutical composition of the present disclosure comprising one or more compounds provided herein or pharmaceutically acceptable salts thereof further comprises one or more additional therapeutically active agents.

The additional therapeutically active agents have complementary activities to the compound provided herein such that they do not adversely affect each other. Such agents are suitably present in combination in amounts that are effective for the purpose intended.

In certain embodiments, the additional therapeutic agent is selected from beta blockers, alpha-agonists, carbonic anhydrase inhibitors, prostaglandin-like compounds, miotic or cholinergic agents, or epinephrine compounds.

Beta blockers reduce the production of aqueous humor. Examples include levobunolol (BETAGAN®), timolol (BETIMOL®, TIMOPTIC®), betaxolol (BETOPTIC®) and metipranolol (OPTIPRANOLOL®).

Alpha-agonists reduce the production of aqueous humor and increase drainage. Examples include apraclonidine (IOPIDINE®) and brimonidine (ALPHAGAN®).

Carbonic anhydrase inhibitors reduce the production of aqueous humor. Examples include dorzolamide (TRUSOPT®) and brinzolamide (AZOPT®).

Prostaglandins and prostaglandin-like compounds increase the outflow of aqueous humor. Examples include latanoprost (XALATAN®), bimatoprost (LUMIGAN®), and travoprost (TRAVATAN™).

Miotic or cholinergic agents increase the outflow of aqueous humor. Examples include pilocarpine (ISOPTO CARPINE®, PILOPINE®) and carbachol (ISOPTO CARBACHOL®).

Epinephrine compounds, such as dipivefrin (PROPINE®), also increase the outflow of aqueous humor.

The additional therapeutic agent or agents may be administered simultaneously or sequentially with the compounds provided herein. Sequential administration includes administration before or after the compounds provided herein. In some embodiments, the additional therapeutic agent or agents may be administered in the same composition as the compounds provided herein. In other embodiments, there may be an interval of time between administration of the additional therapeutic agent and the compounds provided herein.

In some embodiments, the administration of an additional therapeutic agent with a compound provided herein may enable lower doses of the other therapeutic agents and/or administration at less frequent intervals.

### Method for Treatment

Compounds of the present disclosure and pharmaceutical composition comprising the same are capable of inhibiting ROCK, and thus can be useful for inhibiting ROCK activity in a subject in need thereof, and for preventing or treating ROCK-related disorders.

In a further aspect, the present disclosure provides a method of treating ROCK-related disorders, comprising administering an effective amount of the compound or a pharmaceutically acceptable salt thereof or the pharmaceutical composition provided herein to a subject in need thereof.

As used herein, the term “treating”, “treatment” or “therapy” is intended to have its normal meaning of dealing with a disease in order to entirely or partially relieve one, some or all of its symptoms, or to correct or compensate for the underlying pathology, thereby achieving beneficial or desired clinical results. For purposes of this disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treating” can also mean prolonging survival as compared to expected survival if not receiving it. Those in need of therapy include those already with the condition or disorder as well as those prone to have the condition or disorder or those in which the condition or disorder is to be prevented.

As used herein, the term “preventing”, “prevention” or “prophylaxis” is intended to have its normal meaning and includes primary prophylaxis to prevent the development of the disease and secondary prophylaxis whereby the disease has already developed and the patient is temporarily or permanently protected against exacerbation or worsening of the disease or the development of new symptoms associated with the disease.

In some embodiments, the compounds or pharmaceutically acceptable salts thereof and the compositions provided herein may be used for the treatment of a wide variety of ROCK-related disorders including cancer, cardiovascular diseases, autoimmune diseases, ocular diseases, metabolic syndrome, respiratory distress syndrome, kidney disease, overactive bladder, epilepsy, migraine, diabetes, high altitude pulmonary edema, psychiatric disorders, etc.

In certain embodiments, the compounds or pharmaceutically acceptable salts thereof and the compositions provided herein may be used for treating eye disease including glaucoma, ocular hypertension, retinal diseases including Wet AMD, Dry AMD and DME, bone disorder including osteoporosis and osteoarthritis, vascular disease including cerebral vasospasm, coronary vasospasm, hypertension, pulmonary hypertension, high-altitude essential hypertension, sudden death syndrome, angina, myocardial infarction, restenosis, stroke, hypertensive vascular disease, heart failure, cardiac allograft vasculopathy, atherosclerosis, arterial obstruction, peripheral arterial disease, peripheral circulation disorder, vein graft disease, pulmonary disease including chronic obstructive pulmonary disease (COPD) and asthma, neurological disorder including spinal cord injury, dementia, Alzheimer's disease, Parkinson's disease, neuronal degeneration, multiple sclerosis, depression, attention deficit-hyperactivity disorder and neuropathic pain, multiple sclerosis, amyotrophic lateral sclerosis, neovascular disorders, cancer including astrocytic, breast, cervical, colorectal, endometrial, esophageal, gastric, head and neck, hepatocellular, laryngeal, lung, oral, ovarian, prostate and thyroid carcinomas and sarcomas, obesity, urinary organ diseases including overactive bladder (OAB) and benign prostatic hypertrophy (BPH), erectile dysfunction, acute and chronic pain, asthma, spinal cord injury, rheumatoid arthritis, psoriasis, virus infection, myocardial protection.

In certain embodiments, the eye disease that can be treated with the compounds or pharmaceutically acceptable salts thereof and the compositions provided herein is glaucoma.

The concentration and route of administration to the subject will vary depending on the ROCK-related disorders to be treated. In certain embodiments, the administering is conducted via a route selected from the group consisting of parenteral, intraperitoneal, intradermal, intracardiac, intraventricular, intracranial, intracerebrospinal, intrasynovial, intrathecal administration, intramuscular injection, intravitreous injection, intravenous injection, intra-arterial injection, oral, buccal, sublingual, transdermal, topical, intratracheal, intrarectal, subcutaneous, and ocular administration.

### EXAMPLES

The followings further explain the general methods of the present disclosure. The compounds of the present disclosure may be prepared by the methods known in the art. The following illustrates the detailed preparation methods of the preferred compounds of the present disclosure. However, they are by no means limiting the preparation methods of the compounds of the present disclosure.

### Synthetic Examples

For the purpose of illustration, the following examples are included. The Examples provided herein describe the synthesis of compounds disclosed herein as well as intermediates used to prepare the compounds. However, it is to be understood that these examples do not limit the present disclosure and are only meant to suggest a method of practicing the present disclosure. Persons skilled in the art will recognize that the chemical reactions described may be readily adapted to prepare a number of other compounds of the present disclosure, and alternative methods for preparing the compounds of the present disclosure are deemed to be within the scope of the present disclosure. For example, the synthesis of non-exemplified compounds according to the present disclosure may be successfully performed by modifications apparent to those skilled in the art, e.g., by appropriately protecting interfering groups, by utilizing other suitable reagents and building blocks known in the art other than those described, and/or by making routine modifications of reaction conditions. Besides, persons skilled in the art will also understand that individual steps described herein or in the separate batches of a compound may be combined. Alternatively, other reactions disclosed herein or known in the art will be recognized as having applicability for preparing other compounds of the present disclosure. The following description is, therefore, not intended to limit the scope of the present disclosure, but rather is specified by the claims appended hereto.

Abbreviations for chemicals used in the synthesis of the compounds provided herein are listed below:

| | |
|---|---|
| ¹H NMR | Proton Nuclear Magnetic Resonance Spectroscopy |
| (Boc)₂O | Di-tert-butyl dicarbonate |
| aq | aqueous |
| CDCl₃ | deuterated chloroform |
| CDI | 1,l′-Carbonyldiimidazole |
| CH₂Cl₂ | Dichloromethane |
| CH₃CN or MeCN or ACN | Acetonitrile |
| CoCl₂ • 6H₂O | Cobaltous chloride hexahydrate |
| DIAD | Diisopropyl azodicarboxylate |
| DIEA or DIPEA | N,N,-diisopropylethylamine |
| DMAP | 4-Dimethylaminopyridine |
| DMF | dimethyl formamide |
| DMSO | dimethyl sulfoxide |
| DMSO-d₆ | Deuterated dimethyl sulfoxide |
| EDCI | 1-Ethyl-3-(3-dimethylaminopropyl)carbodiimide |
| ee | Enantiomeric excess |
| EtOAc or EA | Ethyl acetate |
| EtOH | Ethanol |
| H₂ | Hydrogen |
| H₂O | Water |
| HATU | (1-[Bis(dimethylamino)methylene]-1H-1,2,3-triazolo[4,5- |
| | b]pyridinium 3-oxide hexafluorophosphate |
| HCl | hydrochloric acid |
| HPLC | High-performance liquid chromatography |
| IPA | Isopropanol |
| K₂CO₃ | potassium carbonate |
| LCMS | Liquid chromatography mass spectrometry |
| LiOH | Lithium hydroxide |
| m/z (ESI) | Mass/charge ratio (Electrospray ionization) |
| MeOH | Methanol |
| CD₃OD | Deuterated methanol |
| MTBE | methyl tert-butyl ether |
| Na₂SO₄ | sodium sulfate |
| NaBH₄ | Sodium borohydride |
| NaCl | Sodium chloride |
| NH₃ | Ammonia |
| NH₄Cl | Ammonium chloride |
| NH₄HCO₃ | Ammonium bicarbonate |
| NMI | N-methylimidazole |
| PE | Petroleum ether |
| Pd(dppf)Cl₂ | [1,1′-Bis(diphenylphosphino)ferrocene]palladium(II) |
| | dichloride |
| Pd(OAc)₂ | Palladium(II) acetate |
| Pd(PPh₃)₄ | Tetrakis(triphenylphosphine)palladium(0) |
| Pd/C | palladium on carbon |
| Pd₂(dba)₃ | Tris(dibenzylideneacetone)dipalladium(0) |
| Prep-TLC | Preparatory thin layer chromatography |
| s, d, t, q, m | Singlet, doublet, triplet, quartet, multiplet |
| SFC | Supercritical fluid chromatography |
| Silica | Silica |
| TBSCl | tert-Butyldimethylsilyl chloride |
| TCFH | Chloro-N,N,N′,N′-tetramethylformamidinium |
| | hexafluorophosphate |
| TEA or Et₃N | Triethylamine |
| TFA | Trifluoroaceti acid |
| THF | Tetrahydrofuran |
| t_{R} | Retention time |
| v/v | Volume/volume |
| XPhos | Dicyclohexyl[2′,4′,6′-tris(propan-2-yl)[1,1′-biphenyl]-2- |
| | yl]phosphane |
| Zn(CN)₂ | Zinc cyanide |

### Synthesis of Intermediates

### Synthesis of Intermediate I-1

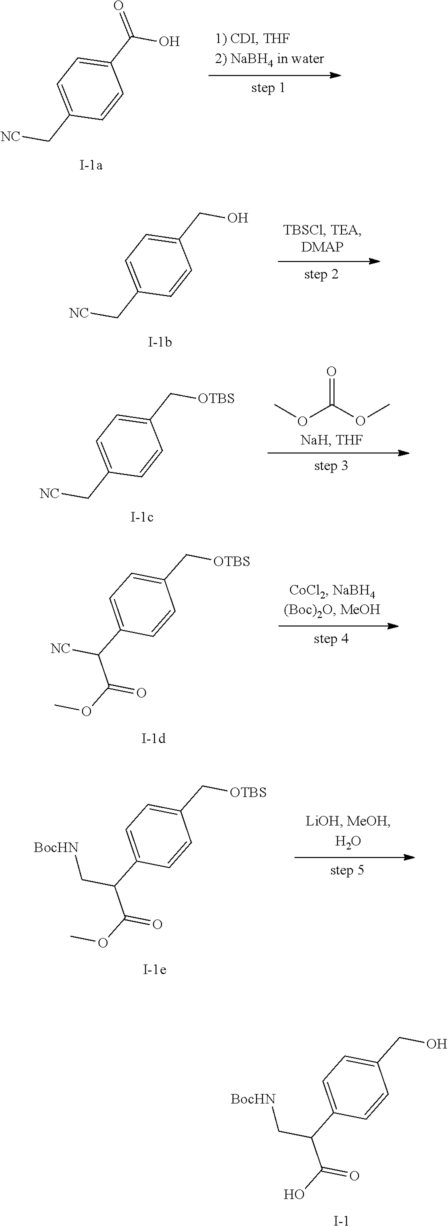

Step 1: A solution of p-cyanomethyl benzoic acid I-1a (20 g, 124.10 mmol, 1 equiv) and CDI (22.14 g, 136.51 mmol, 1.1 equiv) in THF (50 mL) was stirred for 3 h at room temperature under nitrogen atmosphere. Into the mixture was added NaBH₄ (14.08 g, 372.30 mmol, 3 equiv) in H₂O (45 mL) dropwise at 0° C. under nitrogen atmosphere. The resulting mixture was stirred for overnight at room temperature under nitrogen atmosphere. The reaction was quenched with water at room temperature. The resulting mixture was extracted with ethyl acetate (3×500 mL). The combined organic layers were dried over anhydrous Na₂SO₄. After filtration, the filtrate was concentrated under reduced pressure. The residue was purified by silica column chromatography, eluted with CH₂Cl₂/EtOAc (8:1, v/v) to afford 2-[4-(hydroxymethyl)phenyl]acetonitrile I-1b (10.9 g, 59.67%, crude) as a light yellow liquid. ¹H NMR (CD₃OD, 400 MHZ) δ 3.87-3.91 (2H, m), 4.62 (2H, s), 7.37 (4H, q).

Step 2: To a stirred mixture of alcohol I-1b (10.9 g, 74.06 mmol, 1 equiv) and triethylamine (22.48 g, 222.18 mmol, 3 equiv), DMAP (0.09 g, 0.741 mmol, 0.01 equiv) in DCM (100 mL) was added TBSCl (13.39 g, 88.87 mmol, 1.2 equiv) in portions at 0° C. under nitrogen atmosphere. The resulting mixture was stirred for 4 h at room temperature under nitrogen atmosphere. The resulting mixture was diluted with water (260 mL). The resulting mixture was extracted with CH₂Cl₂ (3×100 mL), the combined organic layers were dried over anhydrous Na₂SO₄. After filtration, the filtrate was concentrated under reduced pressure. The residue was purified by prep-TLC (PE/EA=6:1, v/v) to afford 2-(4-(((tert-butyldimethylsilyl)oxy)methyl)phenyl) acetonitrile I-1c (14.0 g, 72.31%) as a light yellow liquid. ¹H NMR (CD₃OD, 400 MHZ) δ 0.13 (6H, s), 0.96 (9H, s), 3.89 (2H, s), 4.77 (2H, s), 7.31-7.40 (4H, m).

Step 3: To a stirred solution of nitrile I-1c (14.0 g, 53.55 mmol, 1 equiv) in THF (20 mL) was added sodium hydride (1.93 g, 80.33 mmol, 1.5 equiv) in portions. The resulting mixture was stirred for 1 h at 0° C. under nitrogen atmosphere. Dimethyl carbonate (19.29 g, 214.20 mmol, 4 equiv) was added to the above mixture in portions at 0° C. under nitrogen atmosphere. The resulting mixture was stirred for overnight at room temperature under nitrogen atmosphere. The reaction was quenched with water (200 ml) at room temperature. The resulting mixture was extracted with ethyl acetate (3×250 mL), the combined organic layers were dried over anhydrous Na₂SO₄. After filtration, the filtrate was concentrated under reduced pressure. The residue was purified by silica column chromatography (PE/EA=6:1, v/v) to afford methyl 2-(4-{[(tert-butyldimethylsilyl)oxy]methyl}phenyl)-2-cyanoacetate I-1d (12.6 g, 73.65%) as a light yellow liquid. ¹H NMR (CD₃OD, 400 MHZ) δ 0.13 (6H, s), 0.97 (9H, s), 3.78 (3H, s), 4.78 (2H, s), 7.37-7.47 (4H, m).

Step 4: Into a 250 mL round-bottom flask were added ester I-1d (12.6 g, 39.44 mmol, 1 equiv) and Boc₂O (34.44 g, 157.76 mmol, 4 equiv), CoCl₂·6H₂O (24.42 g, 102.55 mmol, 2.6 equiv) in methanol (150 mL) at room temperature. NaBH₄ (11.94 g, 315.53 mmol, 8 equiv) was added to the above mixture at 0° C. The resulting mixture was stirred for 15 h at room temperature under nitrogen atmosphere. The combined organic layers were dried over anhydrous Na₂SO₄. After filtration, the filtrate was concentrated under reduced pressure. The residue was purified by silica column chromatography, eluted with PE/EA (0-5.5%) to afford methyl 3-[(tert-butoxycarbonyl)amino]-2-(4-{[(tert-butyldimethylsilyl)oxy]methyl}phenyl)propanoate I-1e (7.1 g, 42.51%) as a light yellow oil. ¹H NMR (DMSO-d₆, 400 MHZ) δ 0.08 (6H, s), 0.91 (9H, s), 1.34 (9H, s), 3.25 (1H, dt), 3.48 (1H, ddd), 3.59 (3H, s), 3.84 (1H, t), 4.68 (2H, s), 6.93 (1H, t), 7.22 (2H, d), 7.27 (2H, d).

Step 5: A solution of ester I-1e (10.1 g, 23.84 mmol, 1 equiv) in methanol (10 mL) and LiOH·H₂O (2.00 g, 47.68 mmol, 2 equiv) in H₂O (6 mL) was stirred for overnight at 60° C. under nitrogen atmosphere. The resulting mixture was concentrated under reduced pressure. The mixture was acidified to pH 6 with HCl (aq.). The residue was purified by reverse flash chromatography with the following conditions: column, C18 silica; mobile phase, methanol in water, 10% to 50% gradient in 40 min; detector, UV 220 nm. This resulted in 3-[(tert-butoxycarbonyl)amino]-2-[4-(hydroxymethyl)phenyl]propanoic acid I-1 (3.58 g, 50.84%) as an off-white solid. LCMS: m/z (ESI), [M+Na]⁺=318.00.

### Synthesis of Intermediate I-2

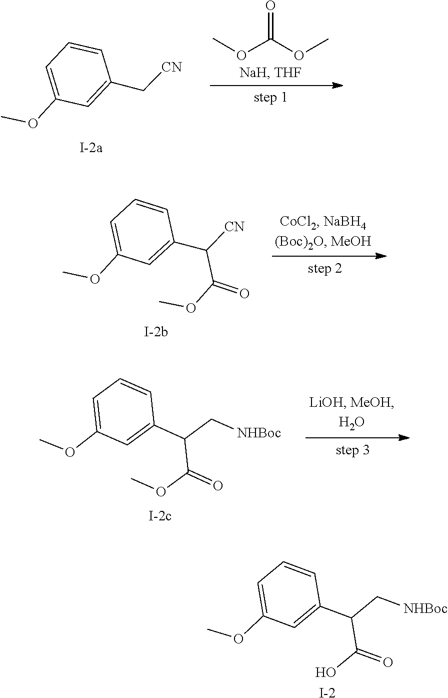

Step 1: To a solution of 2-(3-methoxyphenyl) acetonitrile I-2a (3 g, 20.38 mmol, 1 equiv) in THF (50 mL) was added sodium hydride (0.54 g, 22.42 mmol, 1.1 equiv) at 0° C. The mixture was stirred for 50 min. Dimethyl carbonate (14.69 g, 163.07 mmol, 8 equiv) was added and the mixture and the resulting mixture was allowed to warm to RT and stirred for 2 h. The reaction mixture was quenched by water (50 mL) and extracted with DCM (3×50 mL). The combined organic layers were dried over anhydrous Na₂SO₄. After filtration, the filtrate was concentrated under reduced pressure. The residue was purified by silica column chromatography, eluted with PE/EA (12:1, v/v) to afford methyl 2-cyano-2-(3-methoxyphenyl)acetate I-2b (2.84 g, 67.89%) as a yellow oil. ¹H NMR (400 MHZ, DMSO-d₆) δ 3.76 (d, 6H), 5.63 (s, 1H), 6.81-7.11 (m, 3H), 7.39 (t, 1H).

Step 2: A mixture of ester I-2b (800 mg, 3.90 mmol, 1 equiv), Boc₂O (1701.62 mg, 7.80 mmol, 2 equiv) CoCl₂·6H₂O (1113.06 mg, 4.68 mmol, 1.2 equiv) and NaBH₄ (368.72 mg, 9.75 mmol, 2.5 equiv) in methanol (20 mL) was stirred for overnight at room temperature under nitrogen atmosphere. The resulting mixture was diluted with water (30 mL), extracted with CH₂Cl₂ (3×40 mL), dried over anhydrous Na₂SO₄. After filtration, the filtrate was concentrated under reduced pressure. The residue was purified by prep-TLC (PE/EA=2:1, v/v) to afford methyl 3-[(tert-butoxycarbonyl)amino]-2-(3-methoxyphenyl)propanoate I-2c (504 mg, 41.79%) as a light yellow oil. LCMS: m/z (ESI), [M+Na]⁺=332.00.

Step 3: Into a 50 mL round-bottom flask were added ester I-2c (760 mg, 2.46 mmol, 1 equiv) and LiOH (117.67 mg, 4.91 mmol, 2.0 equiv) in THF (3 mL) and water (3 mL) at room temperature. The resulting mixture was stirred for 3 h at room temperature. The resulting mixture was concentrated under reduced pressure. The mixture was acidified to pH 5 with HCl (1M). The residue was purified by reverse flash chromatography with the following conditions: column, C18 silica; mobile phase, water in methanol, 0% to 100% gradient in 50 min; detector, UV 254 nm. The resulting mixture was concentrated under vacuum, to afford 3-[(tert-butoxycarbonyl)amino]-2-(3-methoxyphenyl)propanoic acid I-2 (200 mg, 27.57%) as a brown yellow solid. LCMS: m/z (ESI), [M-^{t}Bu]⁺=240.25.

### Synthesis of Intermediate I-3

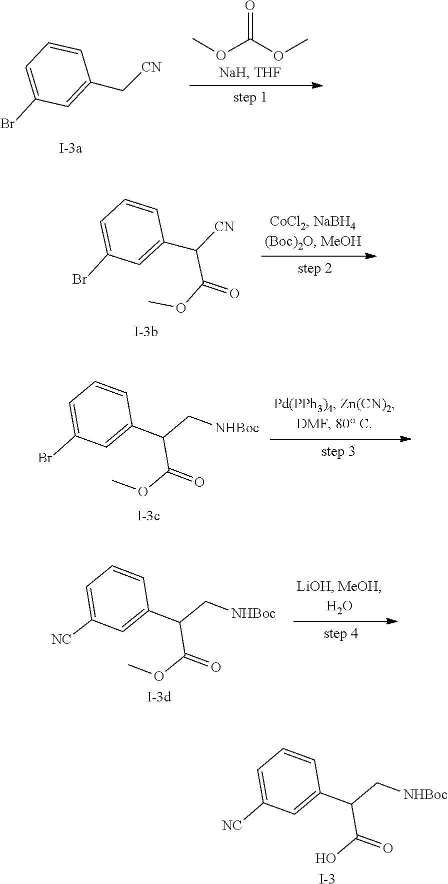

Step 1: To a solution of 2-(3-bromophenyl) acetonitrile I-3a (10 g, 51.01 mmol, 1 equiv) in THF (200 mL) was added sodium hydride (4.08 g, 102.02 mmol, 2 equiv, 60%) at 0° C. The mixture was stirred for 40 min. Dimethyl carbonate (18.38 g, 204.03 mmol, 4 equiv) was added and the mixture was allowed to warm to RT and stirred for 2 h. The resulting mixture was quenched with water at 0° C. (500 mL). The resulting mixture was extracted with ethyl acetate (3×200 mL). The combined organic layers were washed with brine (2×200 mL), dried over anhydrous Na₂SO₄. After filtration, the filtrate was concentrated under reduced pressure. The crude product was re-crystallized from DCM/PE (1:10 v/v, 500 mL) to afford methyl 2-(3-bromophenyl)-2-cyanoacetate I-3b (6.5 g, 50.15%) as a white solid. LCMS: m/z (ESI), [M−H]⁻=253.70. ¹H NMR (DMSO-d₆, 400 MHZ) δ 3.43 (3H, s), 6.67 (1H, ddd), 6.94 (1H, t), 7.39 (1H, d), 7.86 (1H, s).

Step 2: To a stirred mixture of ester I-3b (5.5 g, 21.65 mmol, 1 equiv), CoCl₂·6H₂O (15.45 g, 64.94 mmol, 3 equiv) and Boc₂O (14.17 g, 64.94 mmol, 3 equiv) in methanol (100 mL) was added NaBH₄ (4.91 g, 129.88 mmol, 6 equiv) dropwise at 0° C. under air atmosphere, the mixture was allowed to warm to RT and stirred for 2 h. The resulting mixture was quenched with water at 0° C. (200 mL). The resulting mixture was filtered. The filtered cake was washed with DCM (4×100 mL). The filtrate was extracted with CH₂Cl₂ (2×300 mL). The combined organic layers were dried over anhydrous Na₂SO₄. After filtration, the filtrate was concentrated under reduced pressure. The residue was purified by silica column chromatography, eluted with PE/EA (10:1, v/v) to afford methyl 2-(3-bromophenyl)-3-[(tert-butoxycarbonyl)amino]propanoate I-3c (4 g, 30.95%) as a yellow oil. LCMS: m/z (ESI), [M+H−^{t}Bu]⁺=303.85.

Step 3: A mixture of Pd(PPh₃)₄ (0.16 g, 0.14 mmol, 0.05 equiv), Zn(CN)₂ (0.20 g, 1.68 mmol, 0.6 equiv) and ester I-3c (1 g, 2.79 mmol, 1 equiv) in DMF (15 mL) was stirred for 4 h at 80° C. under nitrogen atmosphere. The resulting mixture was diluted with water (50 mL). The resulting mixture was extracted with ethyl acetate (3×50 mL). The combined organic layers were washed with brine (3×70 mL), dried over anhydrous Na₂SO₄. After filtration, the filtrate was concentrated under reduced pressure. The residue was purified by prep-TLC (PE/EA=3:1, v/v) to afford methyl 3-[(tert-butoxycarbonyl)amino]-2-(3-cyanophenyl)propanoate I-3d (400 mg, 47.08%) as a white solid. LCMS: m/z (ESI), [M+H−^{t}Bu]⁺=248.95. ¹H NMR (400 MHZ, DMSO-d₆) δ 1.31 (9H, s), 3.33-3.42 (1H, m), 3.46-3.58 (1H, m), 3.63 (3H, s), 3.96 (1H, t), 6.96 (1H, t), 7.47-7.65 (2H, m), 7.73 (1H, d), 7.77 (1H, d).

Step 4: A mixture of LiOH·H₂O (86.17 mg, 2.05 mmol, 2.5 equiv) and nitrile I-3d (250 mg, 0.82 mmol, 1 equiv) in THF (5 mL) and H₂O (1 mL) was stirred for 2 h at room temperature under air atmosphere. The resulting mixture was concentrated under reduced pressure to afford 3-[(tert-butoxycarbonyl)amino]-2-(3-cyanophenyl)propanoic acid I-3 (200 mg, 83.87%) as a white solid. LCMS: m/z (ESI), [M+H]⁺=291.00.

### Synthesis of Intermediate I-4

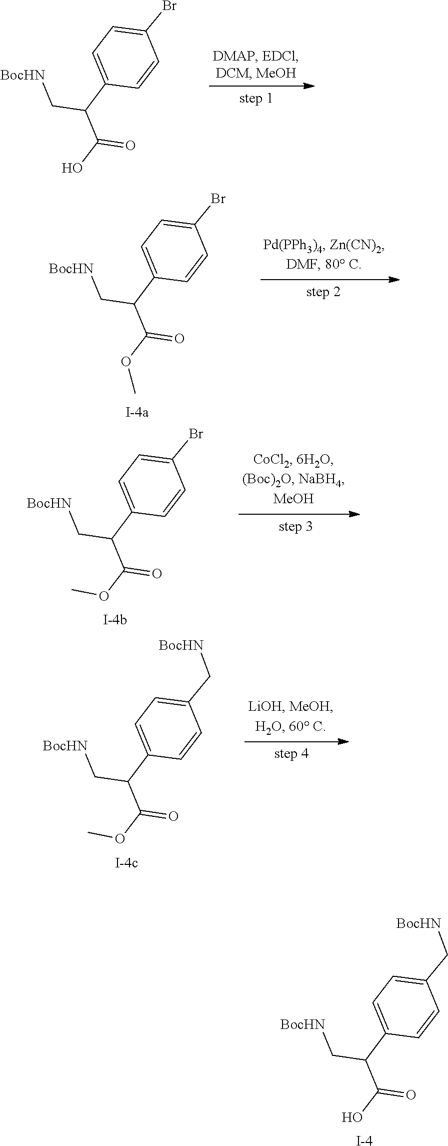

Step 1: To a stirred mixture of 2-(4-bromophenyl)-3-[(tert-butoxycarbonyl)amino]propanoic acid (2.0 g, 5.81 mmol, 1 equiv) and DMAP (141.97 mg, 1.16 mmol, 0.2 equiv) in DCM (10 mL) and methanol (1.0 mL, 24.70 mmol, 4.25 equiv) was added EDCI (2227.74 mg, 11.620 mmol, 2 equiv) in portions at room temperature under nitrogen atmosphere. The resulting mixture was stirred for overnight at room temperature under nitrogen atmosphere. After reaction, the resulting mixture was diluted with water (40 mL). The resulting mixture was extracted with ethyl acetate (3×30 mL). The combined organic layers were washed with water (3×20 mL), dried over anhydrous Na₂SO₄. After filtration, the filtrate was concentrated under reduced pressure. The residue was purified by silica column chromatography, eluted with PE/EA (4:1, v/v) to afford methyl 2-(4-bromophenyl)-3-[(tert-butoxycarbonyl)amino]propanoate I-4a (1.58 g, 75.91%) as a white solid. LCMS: m/z (ESI), [M-t-Bu]⁺=301.80.

Step 2: To a stirred mixture of ester I-4a (1.48 g, 4.13 mmol, 1 equiv) and Zn(CN)₂ (494.81 mg, 4.21 mmol, 1.02 equiv) in DMF (4 mL) was added Pd(PPh₃)₄ (477.42 mg, 0.41 mmol, 0.1 equiv) in portions at room temperature under nitrogen atmosphere. The resulting mixture was stirred for 3 h at 80° C. under nitrogen atmosphere. After cooling to room temperature, the resulting mixture was diluted with water (25 mL). The resulting mixture was extracted with ethyl acetate (3×30 mL). The combined organic layers were dried over anhydrous Na₂SO₄. After filtration, the filtrate was concentrated under reduced pressure. The crude was diluted with DCM (3 mL). The residue was purified by prep-TLC with PE/EA (3:1, v/v) to afford methyl 3-[(tert-butoxycarbonyl)amino]-2-(4-cyanophenyl)propanoate I-4b (1.1 g, 87.48%) as a yellow oil. LCMS: m/z (ESI), [M-t-Bu]⁺=249.30.

Step 3: To a stirred mixture of nitrile I-4b (300 mg, 0.99 mmol, 1 equiv) and (Boc)₂O (430.26 mg, 1.97 mmol, 2 equiv), CoCl₂·6H₂O (469.05 mg, 1.97 mmol, 2 equiv) in methanol (10 mL) was added NaBH₄ (298.32 mg, 7.89 mmol, 8 equiv) in portions at 0° C. under nitrogen atmosphere. The resulting mixture was stirred for 3 h at 25° C. under nitrogen atmosphere. The reaction was quenched with ice water (30 mL) at 0° C. The resulting mixture was filtered and the filtered cake was washed with methanol (3×20 mL). The filtrate was concentrated under reduced pressure. The resulting mixture was extracted with ethyl acetate (3×30 mL). The combined organic layers were washed with water (3×10 mL), dried over anhydrous Na₂SO₄. After filtration, the filtrate was concentrated under reduced pressure. The residue was purified by prep-TLC with PE/EA (5:1, v/v) to afford methyl 3-[(tert-butoxycarbonyl)amino]-2-(4-{[(tert-butoxycarbonyl)amino]methyl}phenyl)propanoate I-4c (170 mg, 42.22%) as a yellow oil. LCMS: m/z (ESI), [M+Na]⁺=431.10.

Step 4: To a stirred mixture of ester I-4c (160 mg, 0.39 mmol, 1 equiv) in methanol (4.0 mL) was added LiOH·H₂O (65.74 mg, 1.57 mmol, 4.0 equiv) in water (2.0 mL) dropwise at 25° C. under nitrogen atmosphere. The resulting mixture was stirred for 3 h at 60° C. under nitrogen atmosphere. After cooling to room temperature, the mixture was acidified to pH 4 with HCl (1 M). The resulting mixture was concentrated under reduced pressure. The residue was purified by reverse flash chromatography with the following conditions: column, C18 silica; mobile phase, MeCN in water, 10% to 50% gradient in 10 min; detector, UV 254 nm, hold for 20 min at 30%. After evaporated the solvent afforded 3-[(tert-butoxycarbonyl)amino]-2-(4-{[(tert-butoxycarbonyl)amino]methyl}phenyl)propanoic acid I-4 (90 mg, 58.25%) as a white solid. LCMS: m/z (ESI), [M−H]=393.15.

### Synthesis of Intermediate I-5

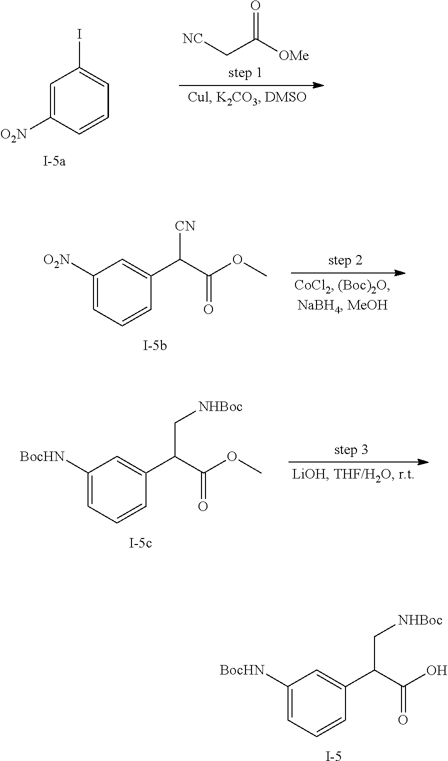

Step 1: Into a 20 mL vial were added 3-iodonitrobenzene I-5a (12.56 g, 50.46 mmol, 1 equiv), K₂CO₃ (20.92 g, 151.38 mmol, 3 equiv), CuI (9.61 g, 50.46 mmol, 1 equiv) and methyl cyanoacetate (5 g, 50.46 mmol, 1 equiv) in DMSO (100 mL) at room temperature. The resulting mixture was stirred for 3 h at 120° C. under nitrogen atmosphere. The resulting mixture was diluted with EA (200 mL), and washed with 1×200 mL of saturated NaHCO₃ (aq.), water (2×200 mL), and saturated brine (1×200 mL). The organic layers were concentrated under reduced pressure. The residue was purified by silica column chromatography, eluted with PE/EA (1:1, v/v). The residue was purified by silica column chromatography, eluted with PE/EA (25:1) to afford methyl 2-cyano-2-(3-nitrophenyl)acetate I-5b (3.0 g, 27.00%) as a red oil. ¹H NMR (DMSO-d₆, 400 MHZ) δ 3.77 (3H, s), 6.01 (1H, s), 7.79 (1H, t), 7.93 (1H, d), 8.30 (1H, dd), 8.34 (1H, t).

Step 2: NaBH₄ (604.77 mg, 15.98 mmol, 8 equiv) was added to a mixture of ester I-5b (440 mg, 2.00 mmol, 1 equiv), CoCl₂·6H₂O (1.24 g, 5.20 mmol, 2.6 equiv) and Boc₂O (1.74 g, 7.99 mmol, 4 equiv) in methanol (10 mL) at 0° C. The resulting mixture was stirred for 1 h at room temperature. The resulting mixture was quenched with water (50 mL) filtered and the filtered cake was washed with DCM (3×10 mL). The filtrate was concentrated under reduced pressure. The resulting mixture was extracted with DCM (3×50 mL). The organic layers were concentrated under reduced pressure. The residue was purified by prep-TLC with PE/EA (2:1, v/v) to afford methyl3-[(tert-butoxycarbonyl)amino]-2-{3-[(tert-butoxycarbonyl)amino]phenyl}propanoate I-5c (300 mg, 38.06%) as a yellow oil. LCMS: m/z (ESI), [M+H]⁺=395.20. ¹H NMR (CD₃OD, 400 MHz) δ 1.43 (9H, s), 1.54 (9H, s), 3.35-3.42 (1H, m), 3.61 (1H, dd), 3.70 (3H, s), 3.85 (1H, dd), 6.94 (1H, d), 7.23 (1H, t), 7.27-7.43 (2H, m).

Step 3: Into a 50 mL round-bottom flask were added ester I-5c (300 mg, 0.76 mmol, 1 equiv), and LiOH (36.43 mg, 1.52 mmol, 2 equiv) in THF (2.5 mL) and water (2.5 mL) at room temperature. The resulting mixture was stirred for 1.5 h at room temperature. The mixture acidified to pH 5 with HCl (1M). The residue was purified by reverse flash chromatography with the following conditions: column, C18 silica; mobile phase, methanol in water, 0% to 20% gradient in 30 min; detector, UV 254 nm. The resulting mixture was concentrated under vacuum to afford 3-[(tert-butoxycarbonyl)amino]-2-{3-[(tert-butoxycarbonyl)amino]phenyl}propanoic acid I-5 (200 mg, 69.12%) as a yellow oil. ¹H NMR (CD₃OD, 400 MHz) δ 1.42 (9H, s), 1.53 (9H, s), 3.40 (1H, dd), 3.48 (1H, dd), 3.61 (1H, dd), 7.03 (1H, d), 7.17 (1H, d), 7.21 (1H, dd), 7.42 (1H, d).

### Synthesis of Intermediate I-6

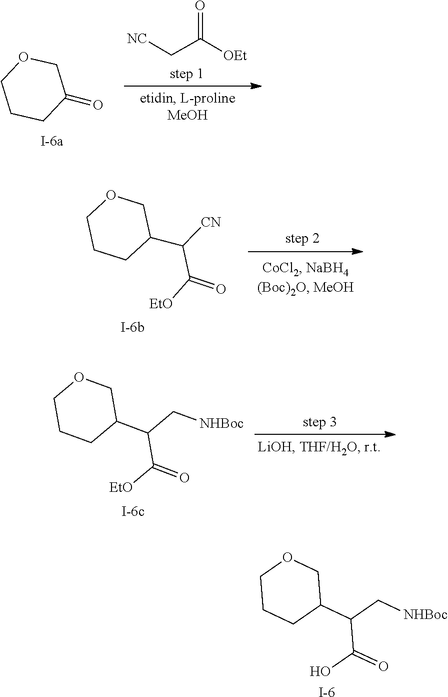

Step 1: Into a 250 mL round-bottom flask were added ethyl cyanoacetate (2.26 g, 19.98 mmol, 1 equiv) and oxan-3-one I-6a (2 g, 19.98 mmol, 1 equiv) and etidin (5.06 g, 19.98 mmol, 1 equiv) and L-proline (0.46 g, 4.00 mmol, 0.2 equiv) and methanol (100 mL) at room temperature. The resulting mixture was stirred for 15 h at 25° C. under nitrogen atmosphere. The resulting mixture was concentrated under reduced pressure. The residue was purified by silica column chromatography, eluted with PE/EA (2:1, v/v) to afford ethyl 2-cyano-2-(oxan-3-yl)acetate I-6b (3.2 g, 81.22%) as a yellow solid. ¹H NMR (CDCl₃, 400 MHz) δ 1.35 (3H, td), 1.47-1.79 (3H, m), 1.85-2.04 (1H, m), 2.29-2.40 (1H, m), 3.36-3.53 (3H, m), 3.88 (1H, ddt), 4.26-4.34 (2H, m).

Step 2: Into a 250 mL round-bottom flask were added ester I-6b (3.1 g, 15.72 mmol, 1 equiv) and Boc₂O (6.86 g, 31.43 mmol, 2 equiv) and CoCl₂·6H₂O (7.48 g, 31.43 mmol, 2 equiv) in methanol (80 mL) at 0° C. NaBH₄ (2.97 g, 78.59 mmol, 5 equiv) was added to the above mixture at 0° C. portion wise. The resulting mixture was stirred for 12 h at room temperature under nitrogen atmosphere. The reaction was quenched by the addition of water (200 mL) at 0° C. The resulting mixture was filtered and the filter cake was washed with DCM (6×50 mL). The aqueous layer was extracted with CH₂Cl₂ (3×200 mL). The resulting mixture was concentrated under reduced pressure. The residue was purified by silica column chromatography, eluted with PE/EA (2:1, v/v) to afford ethyl 3-[(tert-butoxycarbonyl)amino]-2-(oxan-3-yl)propanoate I-6c (3 g, 63.33%) as a yellow oil. LCMS: m/z (ESI), [M+H−^{t}Bu]⁺=246.05. ¹H NMR (CDCl₃, 400 MHz) δ 1.26-1.31 (3H, m), 1.44 (9H, s), 1.63 (2H, dq), 1.78 (1H, d), 1.93 (2H, dh), 2.51 (1H, dtd), 3.15-3.48 (4H, m), 3.77-3.96 (2H, m), 4.13-4.23 (2H, m).

Step 3: Into a 50 mL round-bottom flask were added ester I-6c (1 g, 3.32 mmol, 1 equiv) and LiOH·H₂O (158.93 mg, 6.64 mmol, 2 equiv) and H₂O (4 mL) and methanol (8 mL) at room temperature. The resulting mixture was stirred for 15 h at 60° C. under nitrogen atmosphere. The mixture was acidified to pH 5 with HCl (1M). The resulting mixture was concentrated under reduced pressure. The residue was purified by reverse flash chromatography with the following conditions: column, C18 silica; mobile phase, methanol in water, 0% to 30% gradient in 40 min; detector, UV 220 nm. The resulting mixture was concentrated under reduced pressure. The resulting solid was dried by lyophilization. This resulted in 3-[(tert-butoxycarbonyl)amino]-2-(oxan-3-yl)propanoic acid I-6 (650 mg, 71.67%) as a white solid. LCMS: m/z (ESI), [M+H−^{t}Bu]⁺=218.15. ¹H NMR (CDCl₃, 400 MHz) δ 1.48 (9H, d), 1.65 (2H), 1.74-2.06 (2H, m), 2.51 (1H, s), 3.14 (1H, s), 3.20-3.54 (4H, m), 3.89 (2H, dt), 6.77 (1H, s), 10.29 (1H, s).

### Synthesis of Intermediate I-7

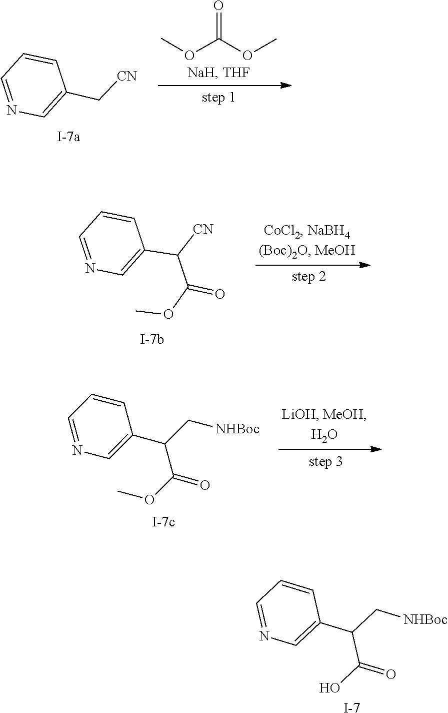

Step 1: Into a 250 mL round-bottom flask were added 2-(pyridin-3-yl) acetonitrile I-7a (6 g, 50.79 mmol, 1 equiv) and THF (40 mL, 493.71 mmol), sodium hydride (2.23 g, 55.87 mmol, 1.1 equiv, 60%) at 0° C. The resulting mixture was stirred for 1 h at room temperature, to the above mixture was add dimethyl carbonate (36.60 g, 406.30 mmol, 8 equiv). The resulting mixture was stirred for 4 h at room temperature. The resulting mixture was extracted with CH₂Cl₂/methanol=20:1 (3×30 mL). The combined organic layers were dried over anhydrous Na₂SO₄. After filtration, the filtrate was concentrated under reduced pressure. The residue was purified by silica column chromatography, eluted with PE/EA (1:1, v/v) to afford methyl 2-cyano-2-(pyridin-3-yl)acetate I-7b (7.4 g, 54.83%) as a brown yellow solid. LCMS: m/z (ESI), [M+H]⁺=177.25.

Step 2: Into a 100 mL round-bottom flask were added ester I-7b (2 g, 11.35 mmol, 1 equiv), CoCl₂·6H₂O (8.10 g, 34.06 mmol, 3 equiv) and Boc₂O (4.96 g, 22.70 mmol, 2 equiv) in methanol (40 mL) was added. NaBH₄ (3.44 g, 90.82 mmol, 8 equiv) at 0° C. The resulting mixture was stirred for 2 h at 0° C. The reaction was quenched with water at room temperature. The resulting mixture was extracted with CH₂Cl₂ (3×30 mL), dried over anhydrous Na₂SO₄. After filtration, the filtrate was concentrated under reduced pressure. The residue was purified by prep-TLC with DCM/EA (1:1, v/v) to afford methyl3-[(tert-butoxycarbonyl)amino]-2-(pyridin-3-yl)propanoate I-7c (310 mg, 8.14%) as a yellow solid. LCMS: m/z (ESI), [M+H]⁺=281.15.

Step 3: Into a 50 mL round-bottom flask were added ester I-7c (300 mg, 1.07 mmol, 1 equiv), LiOH (51.26 mg, 2.14 mmol, 2 equiv), THF (5 mL) and water (5 mL) at room temperature. The residue was purified by reverse flash chromatography with the following conditions: column, C18 silica; mobile phase, MeCN in water, 10% to 50% gradient in 40 min; detector, UV 220 nm. This resulted in 3-[(tert-butoxycarbonyl)amino]-2-(pyridin-3-yl)propanoic acid I-7 (140 mg, 41.16%) as a yellow solid. LCMS: m/z (ESI), [M+H]⁺=267.05.

### Synthesis of Intermediate I-8

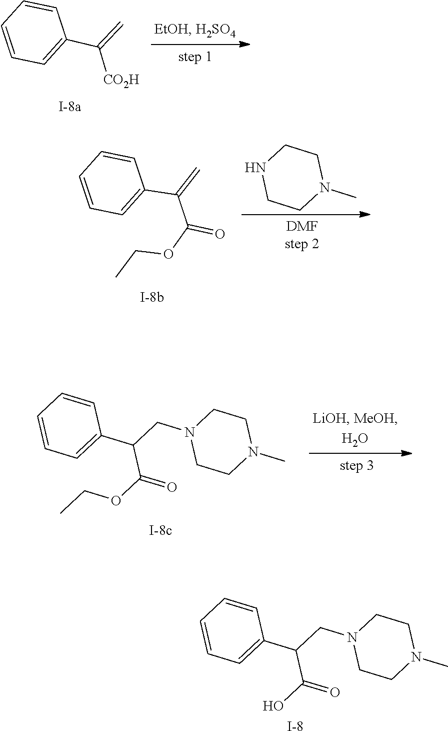

Step 1. Into a 250 mL round-bottom flask were added atropic acid I-8a (8 g, 54.00 mmol, 1 equiv) and ethanol (220 mL), H₂SO₄ (0.5 mL, 9.38 mmol, 0.17 equiv) at room temperature. The resulting mixture was stirred for overnight at 80° C. After cooling to room temperature. The mixture was neutralized to pH 8 with saturated NaHCO₃ (aq.). The resulting mixture was diluted with water (70 mL). The aqueous layer was extracted with ethyl acetate (3×100 mL). The resulting mixture was concentrated under reduced pressure. The residue was purified by silica column chromatography, eluted with PE/EA (10:1, v/v) to afford ethyl 2-phenylprop-2-enoate I-8b (7.5 g, 78.82%) as a colorless liquid. ¹H NMR (400 MHZ, DMSO-d₆) δ 1.3 (t, 3H), 4.2 (q, 2H), 6.0 (d, 1H), 6.2 (d, 1H), 7.2-7.5 (m, 5H).

Step 2: A solution of ester I-8b (250 mg, 1.50 mmol, 1 equiv) and 1-methylpiperazine (568.42 mg, 5.68 mmol, 4 equiv) in DMF (3 mL) was stirred for 1 h at room temperature under nitrogen atmosphere. The residue was dissolved in PE (20 mL). The combined organic layers were washed with water (3×10 mL), dried over anhydrous Na₂SO₄. After filtration, the filtrate was concentrated under reduced pressure afford ethyl 3-(4-methylpiperazin-1-yl)-2-phenylpropanoate I-8c (330 mg, 84.16%) as an off-white solid. LCMS: m/z (ESI), [M+H]⁺=277.25.

Step 3: To a stirred solution of piperazin I-8c (500 mg, 1.81 mmol, 1 equiv) in methanol (10 mL) and H₂O (5 mL) was added LiOH (173.31 mg, 7.24 mmol, 4 equiv) dropwise at room temperature under air atmosphere. The resulting mixture was stirred for 3 h at room temperature under air atmosphere. The resulting mixture was concentrated under reduced pressure. The residue was purified by reverse flash chromatography with the following conditions: column, C18 silica; mobile phase, MeCN in water, 20% to 30% gradient in 10 min; detector, UV 254 nm to afford 3-(4-methylpiperazin-1-yl)-2-phenylpropanoic acid I-8 (220 mg, 48.97%) as a white solid. LCMS: m/z (ESI), [M+H]⁺=249.15.

### Synthesis of Intermediate I-9

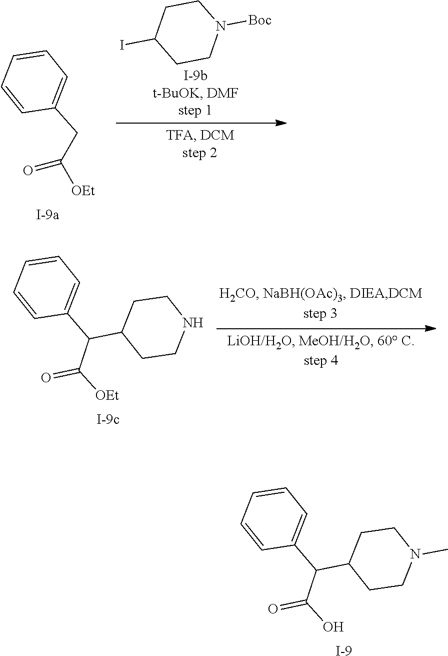

Step 1: To a stirred mixture of ethyl phenylacetate I-9a (1000 mg, 6.09 mmol, 1 equiv) and tert-butyl 4-iodopiperidine-1-carboxylate I-9b (2.84 g, 9.14 mmol, 1.5 equiv) in DMF (10 mL) was added t-BuOK (1.37 g, 12.18 mmol, 2 equiv) in portions at 0° C. under air atmosphere. The resulting mixture was stirred for 16 h at 25° C. under air atmosphere. The reaction was quenched with sat. NH₄Cl (aq.) at 0° C. The resulting mixture was extracted with ethyl acetate (3×50 mL), dried over anhydrous Na₂SO₄. After filtration, the filtrate was concentrated under reduced pressure. The residue was purified by reverse flash chromatography with the following conditions: column, silica; mobile phase, MeCN in water, 50% to 90% gradient in 10 min; detector, UV 254 nm to afford tert-butyl 4-(2-ethoxy-2-oxo-1-phenylethyl) piperidine-1-carboxylate (550 mg, 25.99%) as a yellow solid. ¹H NMR (400 MHZ, DMSO-d₆) δ 1.13 (4H, t), 1.37 (9H, s), 1.67 (1H, d), 2.01-2.16 (1H, m), 3.37 (4H, d), 3.83 (1H, d), 3.90-4.17 (3H, m), 7.25-7.37 (5H, m).

Step 2: To a stirred solution of tert-butyl 4-(2-ethoxy-2-oxo-1-phenylethyl) piperidine-1-carboxylate (500 mg, 1.44 mmol, 1 equiv) in DCM (6 mL) was added TFA (2 mL). The resulting mixture was stirred for 2 h at room temperature under air atmosphere. The resulting mixture was concentrated under vacuum to afford ethyl 2-phenyl-2-(piperidin-4-yl)acetate I-9c (340 mg, 95.52%) as a yellow oil. LCMS: m/z (ESI), [M+H]⁺=248.15.

Step 3: To a stirred mixture of ester I-9c (350 mg, 1.42 mmol, 1 equiv) and formaldehyde (424.89 mg, 14.15 mmol, 10 equiv) in DCM (10 mL) was added DIEA (914.47 mg, 7.08 mmol, 5 equiv). The resulting mixture was stirred for 0.5 h at room temperature under air atmosphere. To the above mixture was added NaBH(OAc)₃ (899.73 mg, 4.25 mmol, 3 equiv). The resulting mixture was stirred for additional 2 h at room temperature. The reaction was quenched by the addition of water (4 mL) at 0° C. The resulting mixture was extracted with ethyl acetate (3×30 mL), dried over anhydrous Na₂SO₄. After filtration, the filtrate was concentrated under reduced pressure. This resulted in ethyl 2-(1-methylpiperidin-4-yl)-2-phenylacetate (350 mg, 94.63%) as a yellow oil. LCMS: m/z (ESI), [M+H]⁺=262.10.

Step 4: To a stirred solution of ethyl 2-(1-methylpiperidin-4-yl)-2-phenylacetate (350 mg, 1.34 mmol, 1 equiv) in THF (4 mL) and H₂O (1 mL) was added LiOH·H₂O (67.43 mg, 1.61 mmol, 1.2 equiv). The resulting mixture was stirred for 16 h at 70° C. under nitrogen atmosphere. The mixture was acidified to pH 6 with HCl (1M). The resulting mixture was concentrated under vacuum. The residue was purified by reverse flash chromatography with the following conditions: column, C18 silica; mobile phase, methanol in water, 10% to 50% gradient in 10 min; detector, UV 254 nm to afford (1-methylpiperidin-4-yl) (phenyl) acetic acid I-9 (240 mg, 76.82%) as a white solid. LCMS: m/z (ESI), [M+H]⁺=234.15. ¹H NMR (400 MHZ, DMSO-d₆) δ 7.41-7.19 (5H, m), 3.18 (1H, d), 2.85 (1H, d), 2.71 (1H, d), 2.19 (3H, t), 1.99 (1H, s), 1.86 (2H, d), 1.76 (1H, d), 1.31 (1H, q), 1.14-0.99 (2H, m).

### Synthesis of Intermediate I-10

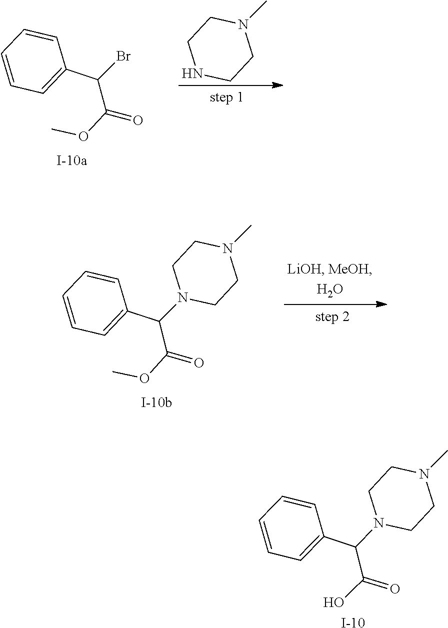

Step 1: A mixture of methyl 2-bromo-2-phenylacetate I-10a (10 g, 43.65 mmol, 1 equiv), 1-methyl-piperazine (13.12 g, 130.96 mmol, 3 equiv) and triethylamine (4.42 g, 43.65 mmol, 1 equiv) in THF (100 mL) was stirred for 4 h at 70° C. under nitrogen atmosphere. The resulting mixture was concentrated under reduced pressure. The residue was purified by silica column chromatography, eluted with CH₂Cl₂/methanol (15:1, v/v) to afford methyl 2-(4-methylpiperazin-1-yl)-2-phenylacetate I-10b (6.3 g, 58.12%) as a brown oil. LCMS: m/z (ESI), [M+H]⁺=249.15.

Step 2: Into a 50 mL round-bottom flask were added ester I-10b (2.7 g, 10.87 mmol, 1 equiv) and LiOH·H₂O (0.91 g, 21.75 mmol, 2.0 equiv) in methanol (16 mL) and water (8 mL) at room temperature. The resulting mixture was stirred for 5 h at room temperature under nitrogen atmosphere. The mixture was acidified to pH 6 with HCl (1 M). The residue was purified by reverse flash chromatography with the following conditions: column, C18 silica; mobile phase, methanol in water, 0% to 5% gradient in 30 min; detector, UV220 nm to afford (4-methylpiperazin-1-yl) (phenyl) acetic acid I-10 (1.6 g, 62.81%) as a white solid. LCMS: m/z (ESI), [M+H]⁺=235.15.

### Synthesis of Intermediate I-11

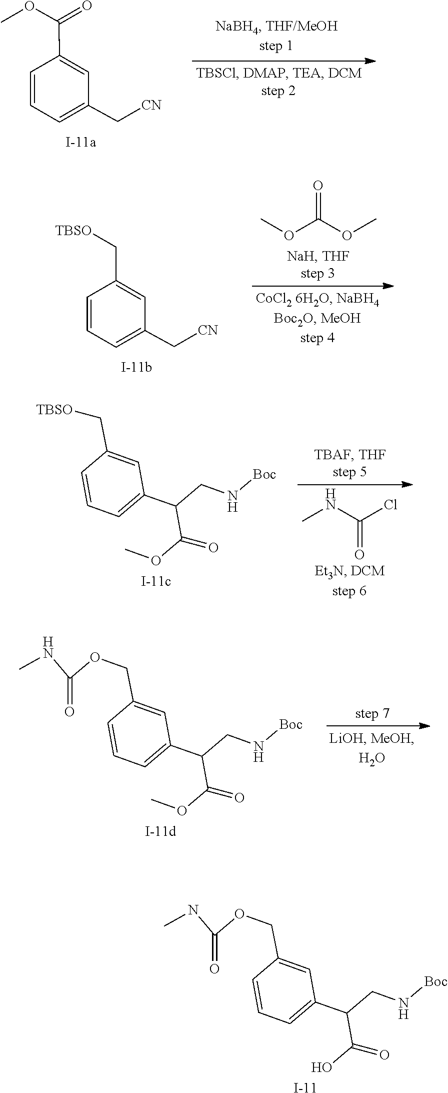

Step 1: To a stirred solution of methyl 3-(cyanomethyl)benzoate I-11a (15 g, 85.62 mmol, 1 equiv) in methanol (250 mL) were added NaBH₄ (16.20 g, 428.12 mmol, 5 equiv) in portions at 0° C. under air atmosphere. The resulting mixture was stirred for overnight at 70° C. under air atmosphere. The mixture was allowed to cool down to room temperature. The resulting mixture was concentrated under reduced pressure. The reaction was quenched by the addition of water (100 mL) at room temperature. The resulting mixture was extracted with ethyl acetate (3×50 mL). The combined organic layers were dried over anhydrous Na₂SO₄. After filtration, the filtrate was concentrated under reduced pressure. The residue was purified by silica column chromatography, eluted with PE/EA (1:10, v/v) to afford 2-[3-(hydroxymethyl)phenyl]acetonitrile (11.14 g, 88.40%) as a yellow oil. ¹H NMR (DMSO-d₆, 400 MHz) δ 4.10 (2H, s), 4.52 (2H, d), 5.28 (1H, t), 7.21 (1H, d), 7.27 (1H, d), 7.32-7.39 (2H, m).

Step 2: To a stirred solution of 2-[3-(hydroxymethyl)phenyl]acetonitrile (11.14 g, 75.69 mmol, 1 equiv) and TBSCl (17.11 g, 113.54 mmol, 1.5 equiv) in DCM (100 mL) were added DMAP (0.92 g, 7.569 mmol, 0.1 equiv) and triethylamine (22.98 g, 227.073 mmol, 3 equiv) dropwise at room temperature under air atmosphere. The resulting mixture was stirred for overnight at room temperature under air atmosphere. The reaction was quenched by the addition of water (100 mL) at room temperature. The resulting mixture was extracted with CH₂Cl₂ (3×100 mL). The combined organic layers were dried over anhydrous Na₂SO₄. After filtration, the filtrate was concentrated under reduced pressure. The residue was purified by silica column chromatography, eluted with PE/EA (1:1, v/v) to afford 2-(3-{[(tert-butyldimethylsilyl)oxy]methyl}phenyl) acetonitrile I-11b (19.5 g, 98.54%) as a colorless oil. ¹H NMR (DMSO-d₆, 400 MHz) δ 0.09 (6H, s), 0.92 (9H, s), 4.04 (2H, s), 4.73 (2H, s), 7.24 (2H, dd), 7.35 (2H, dd).

Step 3: To a stirred solution of nitrile I-11b (19.5 g, 74.59 mmol, 1 equiv) in THF (100 mL) were added sodium hydride (3.58 g, 149.17 mmol, 2 equiv) dropwise at 0° C. under air atmosphere. The resulting mixture was stirred for 1 h at room temperature under air atmosphere. To the above mixture was added dimethyl carbonate (53.75 g, 596.70 mmol, 8 equiv) dropwise at room temperature. The resulting mixture was stirred for additional 3 h at room temperature. The reaction was quenched by the addition of water/ice (100 mL) at room temperature. The resulting mixture was extracted with ethyl acetate (3×100 mL). The combined organic layers were dried over anhydrous Na₂SO₄. After filtration, the filtrate was concentrated under reduced pressure. The residue was purified by silica column chromatography, eluted with PE/EA (1:1, v/v) to afford methyl 2-(3-{[(tert-butyldimethylsilyl)oxy]methyl}phenyl)-2-cyanoacetate (10.1 g, 42.39%) as a yellow oil. ¹H NMR (CD₃OD, 400 MHZ) δ 0.13 (6H, d), 0.97 (9H, s), 3.78 (3H, s), 4.79 (2H, d), 7.22-7.46 (3H, m), 7.50 (1H, dq).

Step 4: To a stirred mixture of methyl 2-(3-{[(tert-butyldimethylsilyl)oxy]methyl}phenyl)-2-cyanoacetate (5 g, 15.65 mmol, 1 equiv) and CoCl₂·6H₂O (11.17 g, 46.95 mmol, 3 equiv) in methanol (100 mL) were added Boc₂O (10.25 g, 46.95 mmol, 3 equiv) at room temperature under air atmosphere. To the above mixture was added NaBH₄ (4.74 g, 125.21 mmol, 8 equiv) in portions at 0° C. The resulting mixture was stirred for additional 3 h at room temperature. The reaction was quenched by the addition of water (100 mL) at room temperature. The resulting mixture was extracted with CH₂Cl₂ (3×50 mL). The combined organic layers were dried over anhydrous Na₂SO₄. After filtration, the filtrate was concentrated under reduced pressure. The residue was purified by silica column chromatography, eluted with PE/EA (1:1, v/v) to afford methyl 3-[(tert-butoxycarbonyl)amino]-2-(3-{[(tert-butyldimethylsilyl)oxy]methyl}phenyl)propanoate I-11c (2282 mg, 34.42%) as a yellow oil. LCMS: m/z (ESI), [M+H-Boc]⁺=324.15. ¹H NMR (CD₃OD, 400 MHZ) δ 0.12 (6H, s), 0.97 (9H, d), 1.43 (9H, d), 3.39 (1H, dd), 3.61 (1H, dd), 3.69 (3H, s), 3.90 (1H, dd), 4.75 (2H, d), 7.19 (1H, d), 7.23-7.34 (3H, m).

Step 5: To a stirred solution of ester I-11c (200 mg, 0.472 mmol, 1 equiv) in THF (5 mL, 61.71 mmol) was added TBAF (370.32 mg, 1.42 mmol, 3 equiv) at room temperature under air atmosphere. The resulting mixture was stirred for 3 h at room temperature under air atmosphere. The resulting mixture was concentrated under reduced pressure. The residue was purified by Prep-TLC with CH₂Cl₂/methanol (40:1, v/v) to afford methyl 3-[(tert-butoxycarbonyl)amino]-2-[3-(hydroxymethyl)phenyl]propanoate (97 mg, 66.41%) as a white solid. LCMS: m/z (ESI), [M+H]⁺=310.20. ¹H NMR (DMSO-d₆, 400 MHz) δ 1.37 (9H, d), 3.23 (1H, dt), 3.50 (1H, ddd), 3.60 (3H, s), 3.86 (1H, dt), 4.48 (2H, t), 5.21 (1H, t), 6.85-7.08 (1H, m), 7.09-7.15 (1H, m), 7.23 (2H, d), 7.30 (1H, t).

Step 6: To a stirred solution of methyl 3-[(tert-butoxycarbonyl)amino]-2-[3-(hydroxymethyl)phenyl]propanoate (300 mg, 0.97 mmol, 1 equiv) in DCM (6 mL, 94.38 mmol, 97.33 equiv) was added N-methylcarbamoyl chloride (108.82 mg, 1.16 mmol, 1.2 equiv) and Et₃N (294.39 mg, 2.91 mmol, 3 equiv) dropwise at room temperature under air atmosphere. The resulting mixture was stirred for overnight at room temperature under air atmosphere. The reaction was quenched by the addition of water (10 mL) at room temperature. The resulting mixture was extracted with CH₂Cl₂ (3×10 mL). The combined organic layers dried over anhydrous Na₂SO₄. After filtration, the filtrate was concentrated under reduced pressure. The residue was purified by reverse flash chromatography with the following conditions: column, C18 silica; mobile phase, MeCN in water, 100% to 100% gradient in 10 min; detector, UV 254 nm to afford methyl 3-[(tert-butoxycarbonyl)amino]-2-(3-{[(methylcarbamoyl)oxy]methyl}-phenyl)propanoate I-11d (90 mg, 25.33%) as a colorless oil. LCMS: m/z (ESI), [M+H]⁺=367.15.

Step 7: To a stirred solution of ester I-11d (270 mg, 0.74 mmol, 1 equiv) in methanol (8 mL) and H₂O (2 mL) were added LiOH (70.59 mg, 2.95 mmol, 4 equiv) at room temperature under air atmosphere. The resulting mixture was stirred for 2 h at room temperature under air atmosphere. The resulting mixture was concentrated under reduced pressure. The residue was purified by reverse flash chromatography with the following conditions: column, C18 silica; mobile phase, MeCN in water, 50% to 60% gradient in 10 min; detector, UV 254 nm to afford 3-[(tert-butoxycarbonyl)amino]-2-(3-{[(methylcarbamoyl)oxy]methyl}phenyl)propanoic acid I-11 (90 mg, 34.66%) as a white solid. LCMS: m/z (ESI), [M+H]⁺=353.20.

### Synthesis of Intermediate I-12

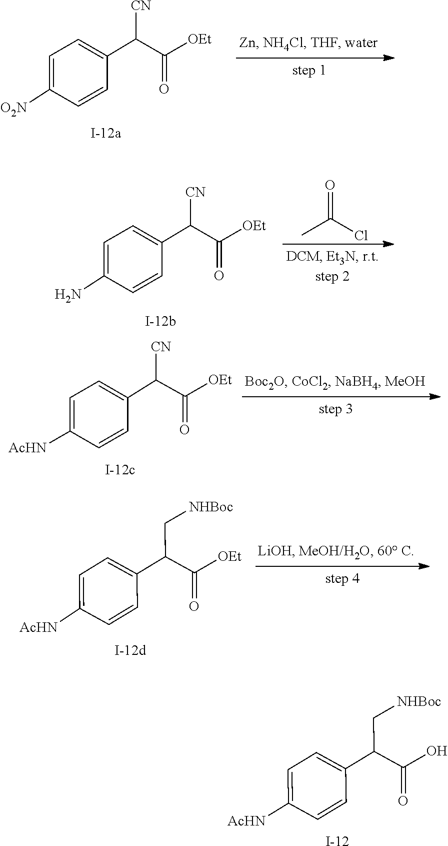

Step 1: To a stirred mixture of ethyl 2-cyano-2-(4-nitrophenyl)acetate I-12a (2.0 g, 8.539 mmol, 1 equiv) and zinc (4.47 g, 68.31 mmol, 8 equiv) in THF (40 mL) was added NH₄Cl (4.57 g, 85.39 mmol, 10 equiv) in water (10 mL) dropwise at 25° C. under nitrogen atmosphere. The resulting mixture was stirred for 48 h at room temperature under nitrogen atmosphere. After reaction, the resulting mixture was filtered, the filter cake was washed with methanol (3×20 mL). The filtrate was concentrated under reduced pressure. The residue was purified by silica column chromatography, eluted with CH₂Cl₂/methanol (60:1, v/v) to afford ethyl 2-(4-aminophenyl)-2-cyanoacetate I-12b (1.2 g, 68.81%) as a yellow solid. LCMS: m/z (ESI), [M+H]⁺=205.15.

Step 2: To a stirred mixture of nitrile I-12b (1.1 g, 5.39 mmol, 1 equiv) and triethylamine (1.64 g, 16.16 mmol, 3 equiv) in DCM (30 mL) was added triethylamine (1.64 g, 16.16 mmol, 3 equiv) dropwise at 0° C. under nitrogen atmosphere. The resulting mixture was stirred for 2 h at room temperature under nitrogen atmosphere. After reaction, the resulting mixture was diluted with water (80 mL). The resulting mixture was extracted with ethyl acetate (3×60 mL). The combined organic layers were washed with saturated brine (3×100 mL), dried over anhydrous Na₂SO₄. After filtration, the filtrate was concentrated under reduced pressure. The residue was dissolved in DCM (5 mL). The residue was purified by prep-TLC with PE/EA (1:1, v/v) to afford ethyl 2-cyano-2-(4-acetamidophenyl)acetate I-12c (1.2 g, 90.47%) as a yellow oil. LCMS: m/z (ESI), [M+H]⁺=247.15. ¹H NMR (DMSO-d₆, 400 MHZ) δ 1.18 (3H, t), 2.06 (3H, s), 4.18 (2H, qq), 5.56 (1H, s), 7.34 (2H, m), 7.65 (2H, m), 10.08 (1H, s).

Step 3: To a stirred mixture of ester I-12c (1.1 g, 4.47 mmol, 1 equiv) and CoCl₂·6H₂O (2.13 g, 8.93 mmol, 2 equiv) in methanol (40 mL) was added (Boc) 20 (1.95 g, 8.93 mmol, 2 equiv) and NaBH₄ (0.68 g, 17.87 mmol, 4 equiv) in portions at 0° C. under air atmosphere. The resulting mixture was stirred for overnight at room temperature under air atmosphere. After reaction, the resulting mixture was filtered, the filter cake was washed with water (3×30 mL). The resulting mixture was diluted with water (50 mL). The resulting mixture was extracted with ethyl acetate (3×40 mL). The combined organic layers were dried over anhydrous Na₂SO₄. After filtration, the filtrate was concentrated under reduced pressure. The residue was purified by prep-TLC with CH₂Cl₂/methanol (5:1, v/v) to afford ethyl 3-[(tert-butoxycarbonyl)amino]-2-(4-acetamidophenyl)propanoate I-12d (900 mg, 57.50%) as a yellow oil. LCMS: m/z (ESI), [M-Boc]⁺=251.15.

Step 4. To a stirred mixture of ester I-12d (500 mg, 2.03 mmol, 1 equiv) in methanol (4.0 mL) was added LiOH (194.51 mg, 8.12 mmol, 4 equiv) in water (1.0 mL) dropwise at room temperature under nitrogen atmosphere. The resulting mixture was stirred for overnight at 60° C. under nitrogen atmosphere. After cooling to room temperature, the resulting mixture was concentrated under vacuum. The resulting mixture was diluted with methanol (3.0 mL). The mixture was acidified to pH 5 with HCl (1 M). The residue was purified by reverse flash chromatography with the following conditions: column, C18 silica; mobile phase, MeCN in water, 10% to 50% gradient in 30 min; detector, UV 254 nm, and hold for 10 min at 16%. After lyophilization to afford 2-(4-acetamidophenyl)-3-((tert-butoxycarbonyl)amino) propanoic acid I-12 (260 mg, 36.54%) as a white solid. LCMS: m/z (ESI), [M-t-Bu]⁺=267.05.

### Synthesis of Intermediate I-13

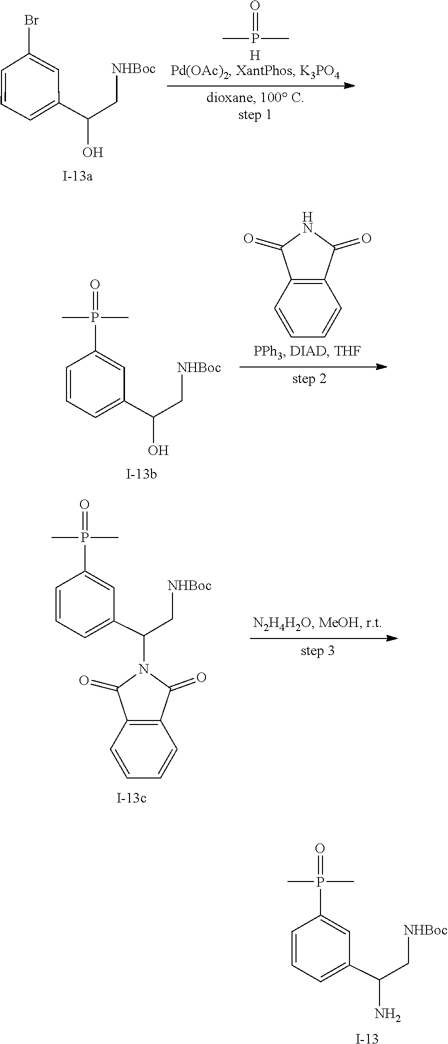

Step 1: A mixture of tert-butyl N-[2-(3-bromophenyl)-2-hydroxyethyl]carbamate I-13a (2.0 g, 6.33 mmol, 1 equiv) and (methylphosphonoyl)methane (0.59 g, 7.59 mmol, 1.2 equiv) in dioxane (25 mL) were added Pd(OAc)₂ (0.28 g, 1.27 mmol, 0.2 equiv), K₃PO₄ (2.69 g, 12.65 mmol, 2.0 equiv) and XantPhos (1.10 g, 1.90 mmol, 0.3 equiv) in portions at 100° C. under nitrogen atmosphere, the mixture were stirred overnight. The mixture cool down to room temperature. The reaction was quenched with water at room temperature. The resulting mixture was extracted with ethyl acetate (3×30 mL). The combined organic layers were dried over anhydrous Na₂SO₄. After filtration, the filtrate was concentrated under reduced pressure. The residue was purified by reverse flash chromatography with the following conditions: column, C18 silica; mobile phase, MeCN in water (0.1% FA), 0% to 100% gradient in 10 min; detector, UV 254 nm. This resulted in tert-butyl N-{2-[3-(dimethylphosphoryl)phenyl]-2-hydroxyethyl}carbamate I-13b (1.3 g, 65.59%) as a yellow oil. ¹H NMR (400 MHZ, DMSO-d₆) δ 1.35 (9H, s), 1.64 (6H, d), 3.11 (2H, m), 4.65 (1H, q), 5.48 (1H, d), 6.78 (1H, t), 7.48 (2H, dd), 7.64 (1H, ddd), 7.72 (1H, d).

Step 2: A mixture of phthalimide (169.05 mg, 1.15 mmol, 1.2 equiv), PPh₃ (753.39 mg, 2.87 mmol, 3 equiv) in THF (1 mL) was treated with DIAD (580.81 mg, 2.87 mmol, 3 equiv) for 0.5 h at 0° C. under nitrogen atmosphere, followed by the addition of alcohol I-13b (300 mg, 0.96 mmol, 1 equiv) in THF (2 mL) dropwise at 0° C. for 1 h. The reaction was quenched with water at room temperature. The resulting mixture was extracted with CH₂Cl₂ (3×20 mL), dried over anhydrous Na₂SO₄. After filtration, the filtrate was concentrated under reduced pressure. The residue was purified by prep-TLC with CH₂Cl₂/methanol (10:1, v/v) to afford tert-butyl N-{2-[3-(dimethylphosphoryl)phenyl]-2-(1,3-dioxoisoindol-2-yl)ethyl}carbamate I-13c (270 mg, 63.74%) as a white solid. ¹H NMR (400 MHZ, DMSO-d₆) δ 1.27 (9H, s), 1.64 (6H, dd), 3.89 (2H, m), 5.43 (1H, dd), 7.27 (1H, t), 7.54 (2H, m), 7.72 (2H, m), 7.87 (4H, m).

Step 3: A solution of phthalimide I-13c (240 mg, 0.54 mmol, 1 equiv) and hydrazine (216.97 mg, 4.34 mmol, 8 equiv) in methanol (5 mL) was stirred for 4 h at room temperature under nitrogen atmosphere. The resulting mixture was filtered, the filter cake was washed with methanol (3×5 mL). The filtrate was concentrated under reduced pressure. The residue was purified by reverse flash chromatography with the following conditions: column, C18 silica; mobile phase, MeCN in water, 10% to 50% gradient in 10 min; detector, UV 254 nm. This resulted in tert-butyl N-{2-amino-2-[3-(dimethylphosphoryl)phenyl]ethyl}-carbamate I-13 (82 mg, 48.40%) as a colorless oil. ¹H NMR (400 MHZ, DMSO-d₆) δ 1.35 (9H, s), 1.63 (6H, d), 3.09 (3H, m), 6.84 (1H, t), 7.54 (4H, m).

### Synthesis of Intermediate I-14

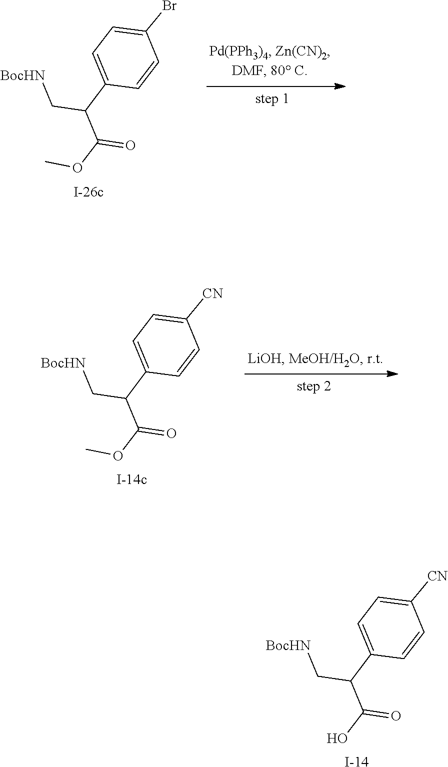

Step 1: To a stirred solution of ester I-26c (2.08 g, 5.81 mmol, 1 equiv) and Pd(PPh₃)₄ (67.10 mg, 0.058 mmol, 0.01 equiv) in DMF (10 mL) was added Zn(CN)₂ (688.59 mg, 5.86 mmol, 1.01 equiv) in portions at room temperature under nitrogen atmosphere. The resulting mixture was stirred for 2.0 h at 80° C. under nitrogen atmosphere. After cooling to room temperature, the resulting mixture was diluted with water (60 mL). The resulting mixture was extracted with ethyl acetate (3×40 mL). The combined organic layers were washed with water (3×10 mL), dried over anhydrous Na₂SO₄. After filtration, the filtrate was concentrated under reduced pressure. The residue was purified by Prep-TLC with PE/EA (2:1, v/v) to afford methyl 3-[(tert-butoxycarbonyl)amino]-2-(4-cyanophenyl)propanoate I-14c (1.3 g, 73.57%) as a yellow solid.

Step 2: To a stirred solution of nitrile I-14c (600 mg, 1.97 mmol, 1 equiv) in methanol (8.0 mL) and LiOH·H₂O (330.89 mg, 7.88 mmol, 4.0 equiv) in water (2.0 mL) was added dropwise at room temperature under nitrogen atmosphere. The resulting mixture was stirred for 2.0 h at 70° C. under nitrogen atmosphere. After cooling to room temperature, the resulting mixture was concentrated under vacuum. After evaporated, the residue was dissolved in methanol (2.0 mL) and basified to pH 9 with NH₃ aq. The residue was purified by reverse flash chromatography with the following conditions: column, C18 silica; mobile phase, MeCN in water, 10% to 50% gradient in 10 min; detector, UV 254 nm, hold for 20 min at 18.2% to afford 3-[(tert-butoxycarbonyl)amino]-2-(4-cyanophenyl)propanoic acid I-14 (400 mg, 69.89%) as a white solid. LCMS: m/z (ESI), [M-^{t}Bu]⁺=235.05.

### Synthesis of Intermediate I-15

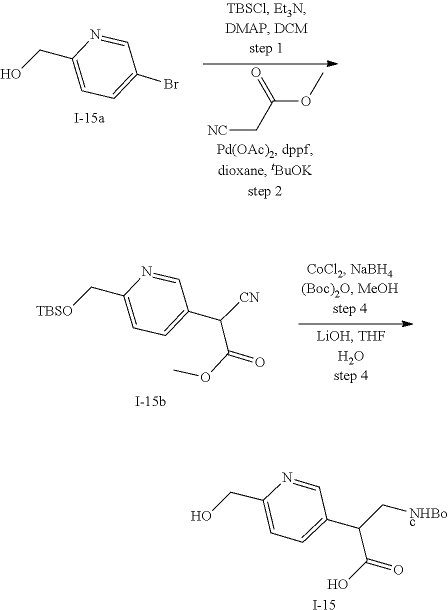

Step 1: Into a 250 mL round-bottom flask were added (5-bromopyridin-2-yl) methanol I-15a (5 g, 26.59 mmol, 1 equiv), TBSCl (4.81 g, 31.91 mmol, 1.2 equiv), triethylamine (8.07 g, 79.78 mmol, 3 equiv), DMAP (0.32 g, 2.66 mmol, 0.1 equiv) and DCM (80 mL) at 0° C. The resulting mixture was stirred for overnight at room temperature. The resulting mixture was extracted with CH₂Cl₂ (3×30 mL), dried over anhydrous Na₂SO₄. After filtration, the filtrate was concentrated under reduced pressure. The residue was purified by silica column chromatography, eluted with EA/CH₂Cl₂ (3:1, v/v) to afford 5-bromo-2-{[(tert-butyldimethylsilyl)oxy]methyl}pyridine (6.33 g, 78.23%) as a yellow oil. LCMS: m/z (ESI), [M+H]⁺=304.05. ¹H NMR (CD₃OD, 400 MHZ) δ 0.15 (6H, s), 0.93 (9H, d), 4.77 (2H, d), 7.50 (1H, m), 8.01 (1H, m), 8.55 (1H, m).

Step 2: Into six 500 mL round-bottom flask were added 5-bromo-2-{[(tert-butyldimethylsilyl)oxy]methyl}pyridine (6.3 g, 20.84 mmol, 1 equiv), methyl cyanoacetate (2.48 g, 25.01 mmol, 1.2 equiv), Pd(OAc)₂ (0.47 g, 2.08 mmol, 0.1 equiv), dppf (2.30 g, 4.17 mmol, 0.2 equiv), t-BuOK (7.02 g, 62.52 mmol, 3 equiv) and 1,4-dioxane (240 mL) at room temperature. The resulting mixture was stirred for 5 h at 70° C. under nitrogen atmosphere. The mixture was quenched with saturated brine (200 mL), extracted with EA (2×200 mL), the combined organic layer was dried over anhydrous Na₂SO₄, filtered and evaporated to afford a crude solid. The residue was purified by silica column chromatography, eluted with PE/EA (1:1, v/v) to afford methyl 2-(6-{[(tert-butyldimethylsilyl)oxy]methyl}pyridin-3-yl)-2-cyanoacetate I-15b (2.4 g, 33.38%) as a yellow oil. LCMS: m/z (ESI), [M+H]⁺=321.15. ¹H NMR (CD₃OD, 400 MHZ) δ 0.17 (6H, s), 0.99 (9H, s), 3.68 (1H, d), 3.83 (3H, s), 4.86 (2H, s), 7.66 (1H, d), 7.98 (1H, dd), 8.56 (1H, d).

Step 3: Into a 50 mL round-bottom flask were added nitrile I-15b (2.24 g, 6.99 mmol, 1 equiv), CoCl₂·6H₂O (2.49 g, 10.49 mmol, 1.5 equiv), Boc₂O (3.05 g, 13.98 mmol, 2.0 equiv) and methanol (20 mL) at room temperature was added NaBH₄ (1.06 g, 27.96 mmol, 4.0 equiv) in portions at 0° C. The resulting mixture was stirred for overnight at room temperature. The reaction was quenched with water (100 mL) at room temperature. The resulting mixture was filtered and the filtered cake was washed with CH₂Cl₂ (3×30 mL). The filtrate was extracted by DCM (2×100 mL), the combined organil layer was dried over anhydrous Na₂SO₄. After filtration, the filtrate was concentrated under reduced pressure. The residue was purified by prep-TLC with CH₂Cl₂/methanol 17:1, v/v) to afford methyl 3-[(tert-butoxycarbonyl)amino]-2-(6-{[(tert-butyldimethylsilyl)oxy]methyl}pyridin-3-yl)propanoate (625 mg, 14.74%) as a brown yellow solid. LCMS: m/z (ESI), [M+H]⁺=425.30.

Step 4: Into a 50 mL round-bottom flask were added methyl 3-[(tert-butoxycarbonyl)amino]-2-(6-{[(tert-butyldimethylsilyl)oxy]methyl}pyridin-3-yl)propanoate (600 mg, 1.41 mmol, 1 equiv), LiOH (67.69 mg, 2.83 mmol, 2 equiv), water (5 mL) and THF (5 mL) at room temperature. The resulting mixture was stirred for overnight at room temperature. The residue was purified by reverse flash chromatography with the following conditions: column, C18 silica; mobile phase, methanol in water, 0 to 10% gradient in 10 min; detector, UV 254 nm. to afford 3-[(tert-butoxycarbonyl)amino]-2-[6-(hydroxymethyl)pyridin-3-yl]propanoic acid I-15 (300 mg, 63.77%) as a light brown solid. LCMS: m/z (ESI), [M+H]⁺=297.10.

### Synthesis of Intermediate I-16

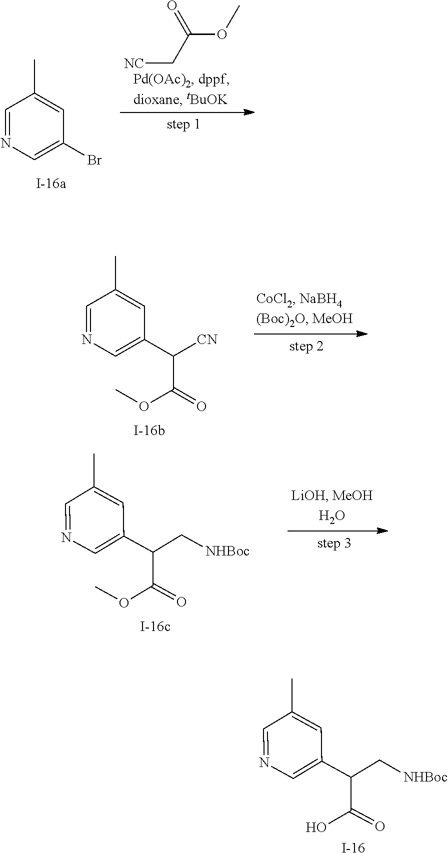

Step 1: A mixture of 3-bromo-5-methylpyridine I-16a (5 g, 29.07 mmol, 1 equiv) and methyl 2-cyanoacetate (3.46 g, 34.88 mmol, 1.2 equiv), Pd(OAc)₂ (0.65 g, 2.91 mmol, 0.1 equiv), dppf (3.21 g, 5.81 mmol, 0.2 equiv), potassium 2-methylpropan-2-olate (9.78 g, 87.20 mmol, 3 equiv) in 1,4-dioxane (100 mL) was stirred for 2 h at 70° C. under nitrogen atmosphere. The reaction was monitored by LCMS. The mixture was acidified to pH 9 with saturated NH₄Cl (aq.). The resulting mixture was stirred for 1 h at room temperature. The resulting mixture was extracted with ethyl acetate (3×300 mL). The combined organic layers were dried over anhydrous Na₂SO₄. After filtration, the filtrate was concentrated under reduced pressure. The residue was purified by silica column chromatography, eluted with CH₂Cl₂/methanol (30:1, v/v) to afford methyl 2-cyano-2-(5-methylpyridin-3-yl)acetate I-16b (710 mg, 12.84%) as a yellow solid. LCMS: m/z (ESI), [M+H]⁺=191.05.

Step 2: To a stirred mixture of nitrile I-16b (800 mg, 4.21 mmol, 1 equiv) and Boc₂O (3671.87 mg, 16.82 mmol, 4 equiv), CoCl₂·6H₂O (2601.83 mg, 10.94 mmol, 2.6 equiv) in methanol (50 mL) was added NaBH₄ (1272.92 mg, 33.65 mmol, 8 equiv) in portions at 0° C. under nitrogen atmosphere. The resulting mixture was stirred for 1 h at room temperature under nitrogen atmosphere. The reaction was quenched with water at room temperature. The resulting mixture was extracted with CH₂Cl₂ (2×100 mL). The combined organic layers were dried over anhydrous Na₂SO₄. After filtration, the filtrate was concentrated under reduced pressure. The residue was purified by prep-TLC with CH₂Cl₂/methanol (32:1, v/v) to afford methyl 3-[(tert-butoxycarbonyl)amino]-2-(5-methylpyridin-3-yl)propanoate I-16c (310 mg, 43.66%) as a yellow solid. LCMS: m/z (ESI), [M+H]⁺=295.15.

Step 3: A solution of ester I-16c (300 mg, 1.02 mmol, 1 equiv) in methanol (3 mL) and LiOH·H₂O (85.53 mg, 2.04 mmol, 2 equiv) in H₂O (1.5 mL) was stirred for 1 h at room temperature under nitrogen atmosphere. The residue was acidified to pH 5 with HCl (1M). The resulting mixture was concentrated under reduced pressure. The residue was purified by reverse flash chromatography with the following conditions: column, C18 silica; mobile phase, methanol in water, 10% to 23% gradient in 30 min; detector, UV 220 nm to afford 3-[(tert-butoxycarbonyl)amino]-2-(5-methylpyridin-3-yl)propanoic acid I-16 (140 mg, 49.00%) as a yellow solid. LCMS: m/z (ESI), [M+H]⁺=281.10.

### Synthesis of Intermediate I-17

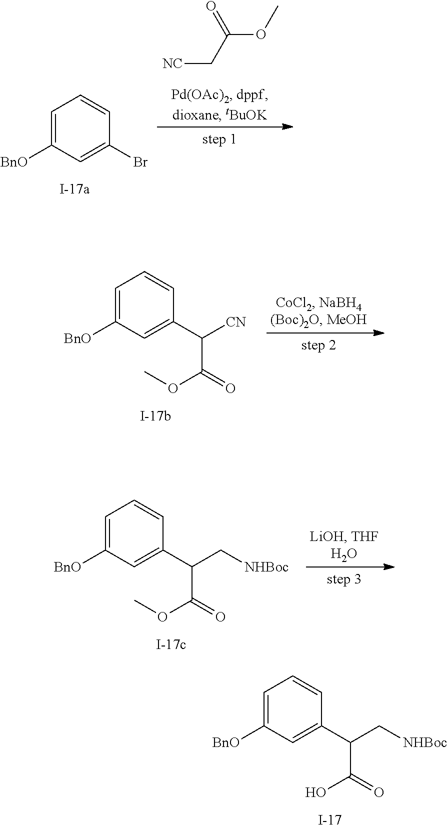

Step 1: Into a 250 mL round-bottom flask were added bromide I-17a (2.26 g, 22.80 mmol, 1.2 equiv), dppf (1.05 g, 1.90 mmol, 0.1 equiv), t-BuOK (6.40 g, 57.01 mmol, 3.0 equiv), palladium acetate (0.21 g, 0.95 mmol, 0.05 equiv) and methyl 2-cyano-2-methylacetate (2.58 g, 22.80 mmol, 1.2 equiv) in dioxane (70 mL) at room temperature. The resulting mixture was stirred for 3 h at 70° C. under nitrogen atmosphere. The residue was quenched with saturated NH₄Cl (100 mL). The resulting mixture was filtered and the filtrate was extracted with ethyl acetate (2×150 mL). The combined organic layers were dried over anhydrous Na₂SO₄, filtered and concentrated under reduced pressure. The residue was purified by silica column chromatography, eluted with PE/EA (10:1, v/v) to afford methyl 2-[3-(benzyloxy)phenyl]-2-cyanoacetate I-17b (1.8 g, 33.67%) as a yellow oil. ¹H NMR (DMSO-d₆, 400 MHz) δ 3.71 (2H, s), 3.73 (3H, s), 4.03 (1H, s), 5.13 (2H, s), 7.01 (1H, dt), 7.03-7.12 (2H, m), 7.31-7.44 (4H, m), 7.45-7.50 (2H, m).

Step 2: Into a 40 mL vial were added nitrile I-17b (2 g, 7.11 mmol, 1 equiv), CoCl₂·6H₂O (4.40 g, 18.49 mmol, 2.6 equiv), and Boc₂O (6.21 g, 28.44 mmol, 4.0 equiv) in methanol (5 mL) at 0° C. NaBH₄ (2.15 g, 56.88 mmol, 8.0 equiv) was added to the above mixture at 0° C. The resulting mixture was stirred for 2 h at room temperature. The reaction was quenched with water at 0° C. The precipitated solids were filtered out and washed with DCM (1×100 mL). The resulting mixture was extracted with DCM (3×100 mL). The combined organic layers were dried over anhydrous Na₂SO₄ filtered and concentrated under reduced pressure. The residue was purified by prep-TLC with CH₂Cl₂/methanol (10:1, v/v) to afford methyl 2-[3-(benzyloxy)phenyl]-3-[(tert-butoxycarbonyl)amino]propanoate I-17c (1.1 g, 40.14%) as a yellow solid. LCMS: m/z (ESI), [M+H−^{t}Bu]⁺=330.10. ¹H NMR (400 MHz, DMSO-d₆) δ 1.35 (9H, s), 3.25 (1H, dt), 3.48 (1H, ddd), 3.59 (3H, s), 3.83 (1H, t), 5.09 (2H, d), 6.83 (1H, d), 6.89 (1H, t), 6.94 (2H, dd), 7.26 (1H, t), 7.38-7.43 (2H, m), 7.46 (2H, d).

Step 3: Into a 50 mL round-bottom flask were added ester I-17c (1.1 g, 2.85 mmol, 1 equiv) in THF (2 mL) and LiOH·H₂O (239.49 mg, 5.71 mmol, 2 equiv) in water (2 mL) at room temperature. The resulting mixture was stirred for 1.5 h at room temperature. The mixture was acidified to pH 5 with HCl (aq.). The residue was purified by reverse flash chromatography with the following conditions: column, C18 silica; mobile phase, water in methanol, 0% to 100% gradient in 30 min; detector, UV 254 nm. The resulting mixture was concentrated under vacuum, to afford 2-[3-(benzyloxy)phenyl]-3-[(tert-butoxycarbonyl)amino]propanoic acid I-17 (500 mg, 47.17%) as a yellow solid. LCMS: m/z (ESI), [M+H−^{t}Bu]⁺=316.10. ¹H NMR (400 MHZ, DMSO-d₆) δ 1.34 (9H, s), 3.23 (1H, dt), 3.44 (1H, ddd), 3.74 (1H, t), 5.08 (2H, d), 6.85 (1H, t), 6.89-6.96 (2H, m), 7.25 (1H, t), 7.31-7.37 (1H, m), 7.37-7.43 (2H, m), 7.46 (2H, d).

### Synthesis of Intermediate I-18

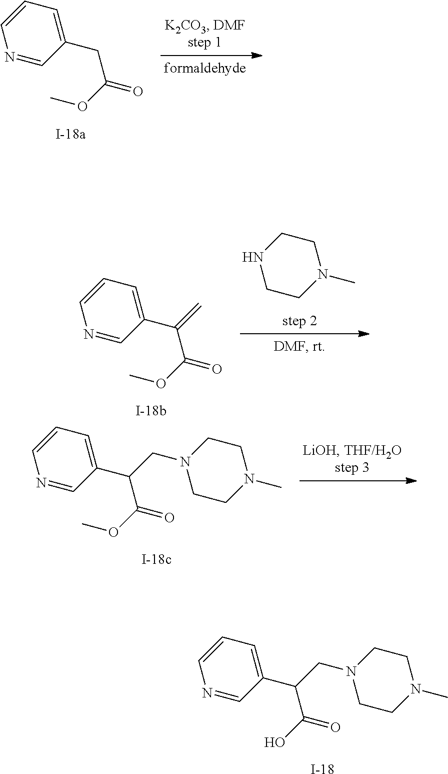

Step 1: A mixture of methyl 2-(pyridin-3-yl)acetate I-18a (4 g, 26.46 mmol, 1 equiv) and K₂CO₃ (10.97 g, 79.38 mmol, 3 equiv) and formaldehyde (1.59 g, 52.92 mmol, 2 equiv) in DMF (40 mL) was stirred for 2 h at 80° C. under nitrogen atmosphere. The mixture was allowed to cool down to room temperature. The reaction was quenched with water (60 mL) at room temperature. The resulting mixture was extracted with ethyl acetate (3×50 mL). The combined organic layers were washed with saturated sodium chloride solution (3×50 mL), dried over anhydrous Na₂SO₄. After filtration, the filtrate was concentrated under reduced pressure. The residue was purified by silica column chromatography, eluted with PE/EA (2:3, v/v) to afford methyl 2-(pyridin-3-yl)prop-2-enoate I-18b (757 mg, 17.53%) as a yellow liquid. LCMS: m/z (ESI), [M+H]⁺=164.25. ¹H NMR (CDCl₃, 400 MHZ) δ 3.87 (3H, s), 6.02 (1H, d), 6.54 (1H, d), 7.34 (1H, ddd), 7.81 (1H, ddd), 8.60 (1H, dd), 8.68 (1H, dd).

Step 2: To a stirred mixture of enolate I-18b (757 mg, 4.64 mmol, 1 equiv) in DMF (10 mL) was added 1-methylpiperazine (2323.41 mg, 23.20 mmol, 5 equiv) at room temperature under nitrogen atmosphere. The resulting mixture was stirred for 2 h at room temperature under nitrogen atmosphere. The residue was purified by reverse flash chromatography with the following conditions: column, C18 silica; mobile phase, MeCN in water, 10% to 50% gradient in 10 min; detector, UV 254 nm. The resulting mixture was concentrated under vacuum. This resulted in methyl 3-(4-methylpiperazin-1-yl)-2-(pyridin-3-yl)propanoate I-18c (884 mg, 72.36%) as a yellow solid. LCMS: m/z (ESI), [M+H]⁺=264.30.

Step 3: To a stirred mixture of piperazin I-18c (150 mg, 0.57 mmol, 1 equiv) in THF (8 mL) and H₂O (2 mL) was added LiOH (68.2 mg, 2.85 mmol, 5 equiv) in portions at room temperature under nitrogen atmosphere. The resulting mixture was stirred for overnight at room temperature under nitrogen atmosphere. The residue was purified by reverse flash chromatography with the following conditions: column, C18 silica; mobile phase, MeCN in water, 10% to 50% gradient in 10 min; detector, UV 254 nm. This resulted in 3-(4-methylpiperazin-1-yl)-2-(pyridin-3-yl)propanoic acid I-18 (120 mg, 84.5%) as a white solid. LCMS: m/z (ESI), [M+H]⁺=250.35.

### Synthesis of Intermediate I-19

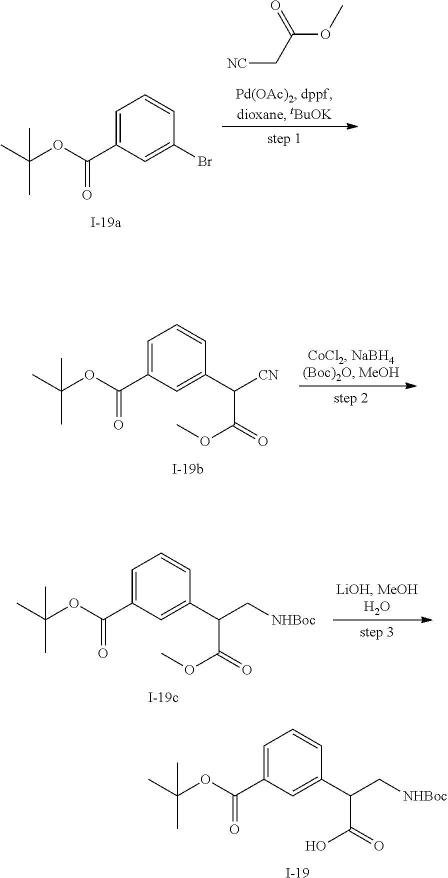

Step 1: Into a 500 mL round bottom flask were added tert-butyl 3-bromobenzoate I-19a (8 g, 31.11 mmol, 1 equiv) and methyl cyanoacetate (4624.44 mg, 46.67 mmol, 1.5 equiv), Pd(OAc)₂ (698.52 mg, 3.11 mmol, 0.1 equiv), dppf (3437.21 mg, 6.22 mmol, 0.2 equiv), t-BuOK (10.47 g, 93.34 mmol, 3 equiv) in dioxane (200 mL) at room temperature. The resulting mixture was stirred for 6 h at 70° C. under nitrogen atmosphere. The mixture was acidified to pH 6 with saturated NH₄Cl (aq.). The resulting mixture was extracted with ethyl acetate (3×50 mL). The combined organic layers were dried over anhydrous Na₂SO₄. After filtration, the filtrate was concentrated under reduced pressure. The residue was purified by silica column chromatography, eluted with PE/EA (7:1, v/v) to afford tert-butyl 3-(1-cyano-2-methoxy-2-oxoethyl)benzoate I-19b (3.7 g, 43.20%) as a light yellow oil. 1H NMR (CD₃OD, 400 MHZ) δ 1.63 (9H, s), 3.81 (3H, s), 7.56 (1H, td), 7.70 (1H, ddd), 8.02 (1H, dt), 8.05-8.09 (1H, m).

Step 2: Into a 100 mL round-bottom flask were added nitrile I-19b (3.7 g, 13.44 mmol, 1 equiv) and CoCl₂·6H₂O (2.62 g, 20.16 mmol, 1.5 equiv), Boc₂O (5.87 g, 26.88 mmol, 2 equiv) in methanol (20 mL) at room temperature. To the above mixture was added NaBH₄ (1.53 g, 40.32 mmol, 3 equiv) in portions over 30 min at 0° C. The resulting mixture was stirred for additional 15 h at room temperature. The reaction was quenched with water at room temperature. The resulting mixture was extracted with DCM (3×40 mL). The combined organic layers were dried over anhydrous Na₂SO₄. After filtration, the filtrate was concentrated under reduced pressure. The residue was purified by silica column chromatography, eluted with PE/EA (5:1, v/v) to afford tert-butyl 3-{3-[(tert-butoxycarbonyl)amino]-1-methoxy-1-oxopropan-2-yl}benzoate I-19c (2.07 g, 40.59%) as a white oil. LCMS: m/z (ESI), [M+H-Boc-^{t}Bu]⁺=223.95.

Step 3: Into a 100 mL round-bottom flask were added ester I-19c (2 g, 5.27 mmol, 1 equiv) and LiOH (252.47 mg, 10.54 mmol, 2 equiv) in water (15 mL) and methanol (15 mL) at room temperature. The resulting mixture was stirred for 2 h at room temperature under nitrogen atmosphere. The mixture was acidified to pH 5 with HCl (1M). The residue was purified by reverse flash chromatography with the following conditions: column, C18 silica; mobile phase, methanol in water, 12% to 22% gradient in 25 min; detector, UV 254 nm. This resulted in 3-[(tert-butoxycarbonyl)amino]-2-[3-(tert-butoxycarbonyl)phenyl]propanoic acid I-19 (650 mg, 33.75%) as an off-white solid. LCMS: m/z (ESI), [M+H]⁺=366.00.

### Synthesis of Intermediate I-20

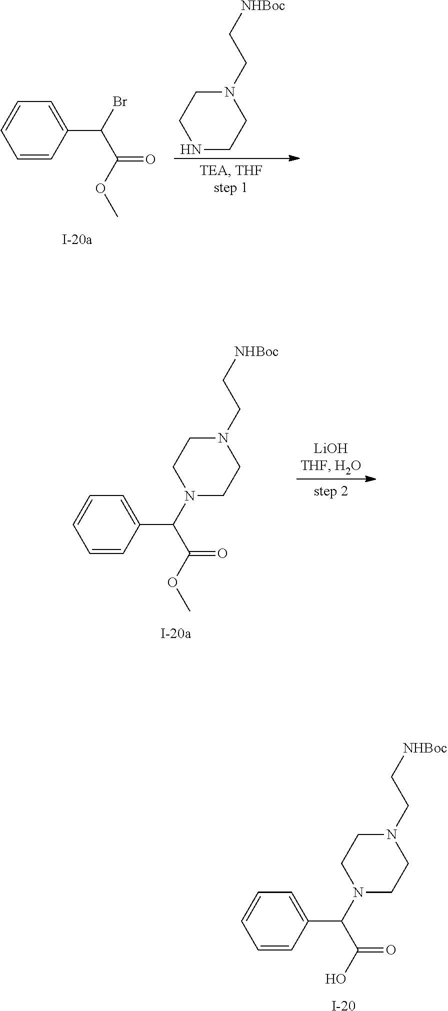

Step 1. Into a 40 mL vial were added methyl 2-bromo-2-phenylacetate I-20a (1 g, 4.37 mmol, 1 equiv) and tert-butyl N-[2-(piperazin-1-yl)ethyl]carbamate (1.20 g, 5.24 mmol, 1.2 equiv), triethylamine (1.33 g, 13.10 mmol, 3 equiv) in THF (20 mL) at room temperature. The resulting mixture was stirred for 15 h at 70° C. under nitrogen atmosphere. The reaction was quenched by the addition of water (60 mL) at room temperature. The resulting mixture was extracted with EA (3×50 mL). The combined organic layers were washed with saturated brine (1×100 mL), dried over anhydrous Na₂SO₄. After filtration, the filtrate was concentrated under reduced pressure. The residue was dissolved in DCM (4 mL). The residue was purified by prep-TLC with PE/EA (1:1, v/v) to afford methyl 2-(4-{2-[(tert-butoxycarbonyl)amino]ethyl}piperazin-1-yl)-2-phenylacetate I-20b (1.54 g, 93.45%) as a light yellow oil. LCMS: m/z (ESI), [M+H]⁺=378.10.

Step 2. Into a 40 mL vial were added piperazin I-20b (600 mg, 1.59 mmol, 1 equiv) and LiOH (76.14 mg, 3.18 mmol, 2 equiv) in THF (5 mL) and water (5 mL) at room temperature. The resulting mixture was stirred for 15 h at room temperature under nitrogen atmosphere. The mixture was acidified to pH 5 with HCl (aq.). The residue was purified by reverse flash chromatography with the following conditions: column, C18 silica; mobile phase, methanol in water, 5% to 10% gradient in 30 min; detector, UV 220 nm. The resulting mixture was concentrated under reduced pressure. This resulted in (4-{2-[(tert-butoxycarbonyl)amino]ethyl}piperazin-1-yl) (phenyl) acetic acid I-20 (469 mg, 81.18%) as an off-white solid. LCMS: m/z (ESI), [M+H]⁺=364.10. ¹H NMR (CD₃OD, 400 MHZ) δ 1.44 (8H, s), 2.63 (2H, s), 2.91 (5H, d), 3.22 (2H, t), 4.40 (1H, s), 7.18-7.49 (3H, m), 7.57 (2H, dd).

### Synthesis of Intermediate I-21

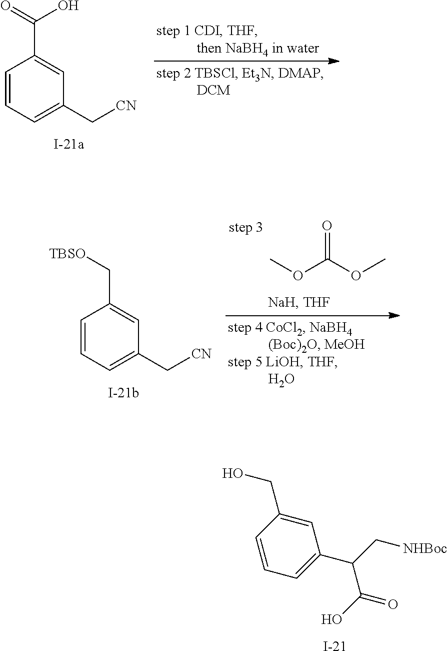

Step 1: A mixture of 3-(cyanomethyl)benzoic acid I-21a (5 g, 31.03 mmol, 1 equiv) and CDI (5.53 g, 34.13 mmol, 1.1 equiv) in THF (26 mL) was stirred at room temperature for 3 h. NaBH₄ (3.52 g, 93.08 mmol, 3 equiv) in H₂O (20 mL) was added to the above mixture dropwise at 0° C. under nitrogen atmosphere. The resulting mixture was stirred for overnight at room temperature under nitrogen atmosphere. The reaction was monitored by TLC with PE/EA (6:1, v/v). The reaction was quenched with water at room temperature. The resulting mixture was extracted with DCM (3×200 mL), the combined organic layer was dried over anhydrous Na₂SO₄. After filtration, the filtrate was concentrated under reduced pressure. The residue was purified by silica column chromatography, eluted with PE/EA (8:1, v/v) to afford 2-[3-(hydroxymethyl)phenyl]acetonitrile (2.3 g, 50.37%) as a light yellow liquid. ¹H NMR (CD₃OD, 400 MHZ) δ 3.91 (2H, s), 4.64 (2H, s), 7.00-7.07 (1H, m), 7.17 (1H, t), 7.39 (1H, q), 7.99 (1H, s).

Step 2: To a stirred mixture of 2-[3-(hydroxymethyl)phenyl]acetonitrile (2.3 g, 15.63 mmol, 1 equiv) and triethylamine (4.74 g, 46.88 mmol, 3 equiv), DMAP (0.02 g, 0.16 mmol, 0.01 equiv) in DCM (10 mL) was added TBSCl (2.83 g, 18.75 mmol, 1.2 equiv) in portions at 0° C. under nitrogen atmosphere. The resulting mixture was stirred for overnight at room temperature under nitrogen atmosphere. The reaction was monitored by TLC with PE/EA (6:1, v/v). The reaction was quenched with water at room temperature. The resulting mixture was extracted with CH₂Cl₂ (3×100 mL), the combined organic layer was dried over anhydrous Na₂SO₄. After filtration, the filtrate was concentrated under reduced pressure. The residue was purified by prep-TLC with PE/EA (6:1, v/v) to afford 2-(3-{[(tert-butyldimethylsilyl)oxy]methyl}phenyl) acetonitrile I-21b (2.1 g, 51.40%) as a light yellow liquid. ¹H NMR (CD₃OD, 400 MHZ) δ 0.13 (6H, s), 0.97 (9H, s), 3.91 (2H, s), 4.78 (2H, s), 7.25 (1H, d), 7.30 (1H, d), 7.36 (1H, d), 7.38 (1H, d).

Step 3. To a stirred mixture of nitrile I-21b (2.1 g, 8.03 mmol, 1 equiv) in THF (25 mL) was added sodium hydride (289.14 mg, 12.05 mmol, 1.5 equiv) in portions at 0° C. under nitrogen atmosphere. The resulting mixture was stirred for 1 h at room temperature under nitrogen atmosphere. Next added dimethyl carbonate (2.89 g, 32.13 mmol, 4 equiv) in portions at 0° C. under nitrogen atmosphere. The final reaction mixture was stirred for overnight at room temperature. The reaction was monitored by TLC with PE/EA (6:1, v/v). The reaction was quenched with water at room temperature. The resulting mixture was extracted with ethyl acetate (3×150 mL), the combined organic layer was dried over anhydrous Na₂SO₄. After filtration, the filtrate was concentrated under reduced pressure. The residue was purified by prep-TLC with PE/EA (6:1, v/v) to afford methyl 2-(3-{[(tert-butyldimethylsilyl)oxy]methyl}phenyl)-2-cyanoacetate (1.9 g, 74.04%) as a light yellow liquid. ¹H NMR (CD₃OD, 400 MHZ) δ 0.13 (6H, s), 0.97 (9H, s), 3.78 (3H, s), 4.80 (2H, q), 7.32-7.51 (4H, m).

Step 4. To a stirred mixture of methyl 2-(3-{[(tert-butyldimethylsilyl)oxy]methyl}phenyl)-2-cyanoacetate (1.9 g, 5.95 mmol, 1 equiv) and (Boc)₂O (5.19 g, 23.79 mmol, 4 equiv) and CoCl₂·6H₂O (8.9 g, 8.96 mmol, 1.5 equiv) in methanol (50 mL) was added NaBH₄ (1.80 g, 47.58 mmol, 8 equiv) in portions at 0° C. under nitrogen atmosphere. The resulting mixture was stirred for overnight at room temperature under nitrogen atmosphere. The resulting mixture was filtered. The filtrate was concentrated under reduced pressure. The residue was purified by prep-TLC with PE/EA (5:1, v/v) to afford methyl 3-[(tert-butoxycarbonyl)amino]-2-(3-{[(tertbutyldimethylsilyl)oxy]methyl}phenyl)propanoate (1.2 g, 47.63%) as a light yellow liquid. LCMS: m/z (ESI), [M+H]⁺=324.20.

Step 5. To A solution of methyl 3-[(tert-butoxycarbonyl)amino]-2-(3-{[(tert-butyldimethylsilyl)oxy]methyl}phenyl)propanoate (1.2 g, 2.83 mmol, 1 equiv) in methanol (1 mL) and LiOH·H₂O (237.72 mg, 5.67 mmol, 2 equiv) in H₂O (1 mL) was stirred for overnight at 60° C. under nitrogen atmosphere. The mixture was acidified to pH 5 with HCl (1M). The resulting mixture was concentrated under vacuum. The residue was purified by reverse flash chromatography with the following conditions: column, C18 silica; mobile phase, methanol in water, 0% to 5% gradient in 30 min; detector, UV 220 nm. This resulted in 3-[(tert-butoxycarbonyl)amino]-2-[3-(hydroxymethyl)phenyl]propanoic acid I-21 (530 mg, 63.35%) as an off-white solid. LCMS: m/z (ESI), [M-^{t}Bu]⁺=240.06.

### Synthesis of Intermediate I-22

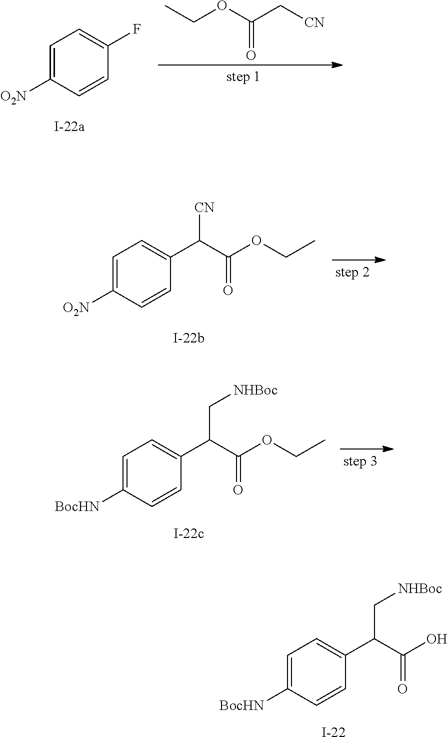

Step 1: A solution of ethyl cyanoacetate (10 g, 88.41 mmol, 1.00 equiv) in DMF (50 ml) was added sodium hydride (2.55 g, 106.09 mmol, 1.2 equiv) portion wise, and the resulting mixture was stirred for 1 h at 0° C. under nitrogen atmosphere followed by the addition of 4-fluoronitrobenzene I-22a (8.73 g, 61.87 mmol, 0.70 equiv) in dioxane (50 ml) dropwise at 0° C. The resulting mixture was stirred for 12 h at 80° C. The resulting mixture was concentrated under reduced pressure. The residue was dissolved in DCM (300 mL). The resulting mixture was washed with 2×250 mL of HCl (1M) and 2×250 ml of water and 1×250 mL of saturated brine. The resulting mixture was concentrated under reduced pressure. The residue was purified by silica column chromatography, eluted with PE/EA (8:1, v/v) to afford ethyl 2-cyano-2-(4-nitrophenyl)acetate I-22b (11.8 g, 55.37%) as a red oil. LCMS: m/z (ESI), [M+H]⁺=235.10. ¹H NMR (400 MHZ, DMSO-d₆) δ 1.20 (3H, t), 4.15 (2H, m), 5.97 (1H, s), 7.74 (2H, d), 8.29 (2H, d).

Step 2: A solution of nitrile I-22b (1 g, 4.54 mmol, 1 equiv) in methanol was treated with Boc₂O (3.96 g, 18.17 mmol, 4 equiv) and CoCl₂·6H₂O (2.81 g, 11.81 mmol, 2.6 equiv) for 5 min at 0° C. under nitrogen atmosphere followed by the addition of NaBH₄ (1.37 g, 36.34 mmol, 8 equiv) in portions at 0° C. The resulting mixture was stirred for 2 h at room temperature. The reaction was quenched with water at room temperature. The resulting mixture was filtered and the filtered cake was washed with methanol (3×20 mL). The aqueous layer was extracted with CH₂Cl₂ (3×100 mL). The resulting mixture was concentrated under reduced pressure. The residue was purified by prep-TLC with CH₂Cl₂/methanol (2:1, v/v) to afford ethyl 3-((tert-butoxycarbonyl)amino)-2-(4-((tert-butoxycarbonyl)amino)phenyl)propanoate I-22c (500 mg, 27.91%) as a yellow oil. LCMS: m/z (ESI), [M+H]⁺=409.25.

Step 3: Into a 100 mL round-bottom flask were added ester I-22c (500 mg, 1.27 mmol, 1 equiv) and LiOH·H₂O (106.37 mg, 2.54 mmol, 2 equiv) in THF (5 mL) and H₂O (5 mL) at room temperature. The resulting mixture was stirred for 15 h at room temperature under air atmosphere. The mixture was acidified to pH 5 with HCl (1M). The resulting mixture was concentrated under reduced pressure. The resulting oil was dried by lyophilization. This resulted in 3-[(tert-butoxycarbonyl)amino]-2-{4-[(tert-butoxycarbonyl)amino]phenyl}propanoic acid I-22 (420 mg, 68.74%) as a yellow solid. LCMS: m/z (ESI), [M−H]⁻=379.15.

### Synthesis of Intermediate I-23

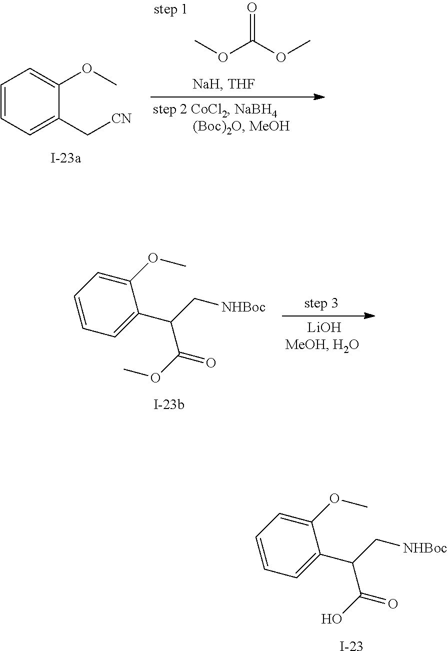

Step 1: A mixture of 2-(2-methoxyphenyl) acetonitrile I-23a (3 g, 20.38 mmol, 1 equiv) and sodium hydride (391.04 mg, 16.31 mmol, 1.2 equiv) in THF (30 mL) was stirred for 2 h at room temperature under nitrogen atmosphere. To the above mixture were added dimethyl carbonate (4.60 g, 50.96 mmol, 2.5 equiv) dropwise at room temperature under nitrogen atmosphere. The resulting mixture was stirred for 2 h at room temperature under nitrogen atmosphere. The resulting mixture was quenched with water (40 mL). The aqueous layer was extracted with CH₂Cl₂ (3×20 mL), dried over anhydrous Na₂SO₄. The resulting mixture was concentrated under reduced pressure. The crude product was purified by reverse phase flash to afford methyl 2-cyano-2-(2-methoxyphenyl)acetate (900 mg, 43.03%) as a yellow solid. LCMS: m/z (ESI), [M+H]⁺=206.21. ¹H NMR (400 MHZ, DMSO-d₆) δ 3.72 (3H, s), 3.83 (3H, d), 7.01 (1H, t), 7.12 (1H, d), 7.34 (1H, d), 7.42 (1H, d).

Step 2: To a stirred mixture of methyl 2-cyano-2-(2-methoxyphenyl)acetate (1.9 g, 9.26 mmol, 1 equiv) and Boc₂O (4.04 g, 18.52 mmol, 2 equiv) in methanol was added CoCl₂ (3.01 g, 23.15 mmol, 2.5 equiv) and NaBH₄ (2.80 g, 74.07 mmol, 8 equiv) in portions at room temperature under nitrogen atmosphere. The resulting mixture was stirred for 2 h at room temperature under nitrogen atmosphere. The resulting mixture was quenched with water (30 mL). The aqueous layer was extracted with CH₂Cl₂ (3×20 mL), dried over anhydrous Na₂SO₄. The resulting mixture was concentrated under reduced pressure. The crude product was purified by reverse phase flash with to afford methyl 3-[(tert-butoxycarbonyl)amino]-2-(2-methoxyphenyl)propanoate I-23b (1.3 g, 45.39%) as a yellow solid. LCMS: m/z (ESI), [M+H]⁺=310.10. ¹H NMR (400 MHZ, DMSO-d₆) δ 1.34 (9H, s), 3.20 (1H, d), 3.50 (1H, td), 3.56 (3H, s), 3.76 (3H, s), 4.11 (1H, t), 6.79 (1H, t), 6.91 (1H, t), 6.99 (1H, d), 7.15 (1H, d), 7.26 (1H, d).

Step 3: A mixture of methyl 3-[(tert-butoxycarbonyl)amino]-2-(2-methoxyphenyl)propanoate (1.3 g, 4.20 mmol, 1 equiv) and LiOH (0.40 g, 16.81 mmol, 4 equiv) in methanol (4 mL) and water (1 mL) was stirred for 2 h at room temperature under nitrogen atmosphere. The resulting mixture was concentrated under reduced pressure. The crude product was purified by reverse phase flash to afford 3-[(tert-butoxycarbonyl)amino]-2-(2-methoxyphenyl)propanoic acid I-23 (800 mg, 64.46%) as a yellow solid. ¹H NMR (400 MHz, DMSO-d₆) 1.33 (9H, s), 3.08-3.24 (2H, m), 3.75 (4H, s), 6.61 (1H, s), 6.78-6.87 (1H, m), 6.84-6.97 (1H, m), 7.13 (1H, td), 7.22 (1H, dd).

### Synthesis of Intermediate I-24

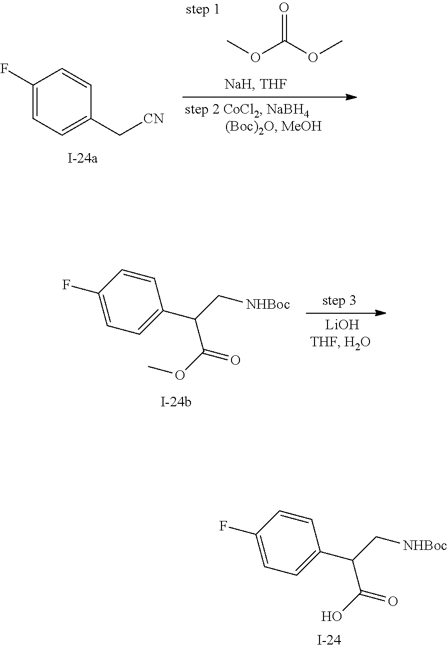

Step 1: A solution of 4-fluorobenzeneacetonitrile I-24a (3 g, 22.20 mmol, 1 equiv) in THF was treated with sodium hydride (1.1 g, 45.84 mmol, 2.06 equiv) for 1 h at 0° C. under nitrogen atmosphere followed by the addition of dimethyl carbonate (4.40 g, 48.84 mmol, 2.2 equiv) in portions at room temperature. The resulting mixture was stirred for 12 h at room temperature. The reaction was quenched with water at room temperature. The resulting mixture was extracted with ethyl acetate (3×30 mL), dried over anhydrous Na₂SO₄. After filtration, the filtrate was concentrated under reduced pressure. The residue was purified by prep-TLC with PE/EA (3:1, v/v) to afford methyl 2-cyano-2-(4-fluorophenyl)acetate (1.1 g, 15.01%) as a yellow oil. ¹H NMR (DMSO-d₆, 400 MHZ) δ 3.74 (3H, s), 5.73 (1H, s), 7.25-7.35 (2H, m), 7.46-7.55 (2H, m).

Step 2: A mixture of methyl 2-cyano-2-(4-fluorophenyl)acetate (1 g, 5.18 mmol, 1 equiv), Boc₂O (2.26 g, 10.36 mmol, 2.00 equiv) and CoCl₂·6H₂O (2.46 g, 10.34 mmol, 2.00 equiv) in methanol was stirred for 5 min at 0° C. under nitrogen atmosphere. Into the mixture added NaBH4 (1.57 g, 41.42 mmol, 8 equiv) at 0° C.˜rt. The resulting mixture was filtered and the filter cake was washed with CH₂Cl₂ (3×30 mL). The filtrate was concentrated under reduced pressure. The residue was purified by prep-TLC with PE/EA (5:1, v/v) to afford methyl 3-[(tert-butoxycarbonyl)amino]-2-(4-fluorophenyl)propanoate I-24b (400 mg, 25.68%) as a yellow oil. LCMS: m/z (ESI), [M+H]⁺=298.25.

Step 3. Into a 50 mL round-bottom flask were added methyl 3-[(tert-butoxycarbonyl)amino]-2-(4-fluorophenyl)propanoate I-24b (380 mg, 1.28 mmol, 1 equiv), LiOH (61.22 mg, 2.56 mmol, 2.00 equiv) and THF (2 mL), water (1 mL) at room temperature. The resulting mixture was stirred for 8 h at 60° C. The mixture was acidified to pH 4-6 with conc. HCl. The residue was purified by reverse flash chromatography with the following conditions: column, C18 silica; mobile phase, methanol in water, 10% to 50% gradient in 10 min; detector, UV 254 nm. This resulted in 3-[(tert-butoxycarbonyl)amino]-2-(4-fluorophenyl)propanoic acid I-24 (260 mg, 63.98%) as a yellow solid. LCMS: m/z (ESI), [M−56]⁺=228.05.

### Synthesis of Intermediate I-25

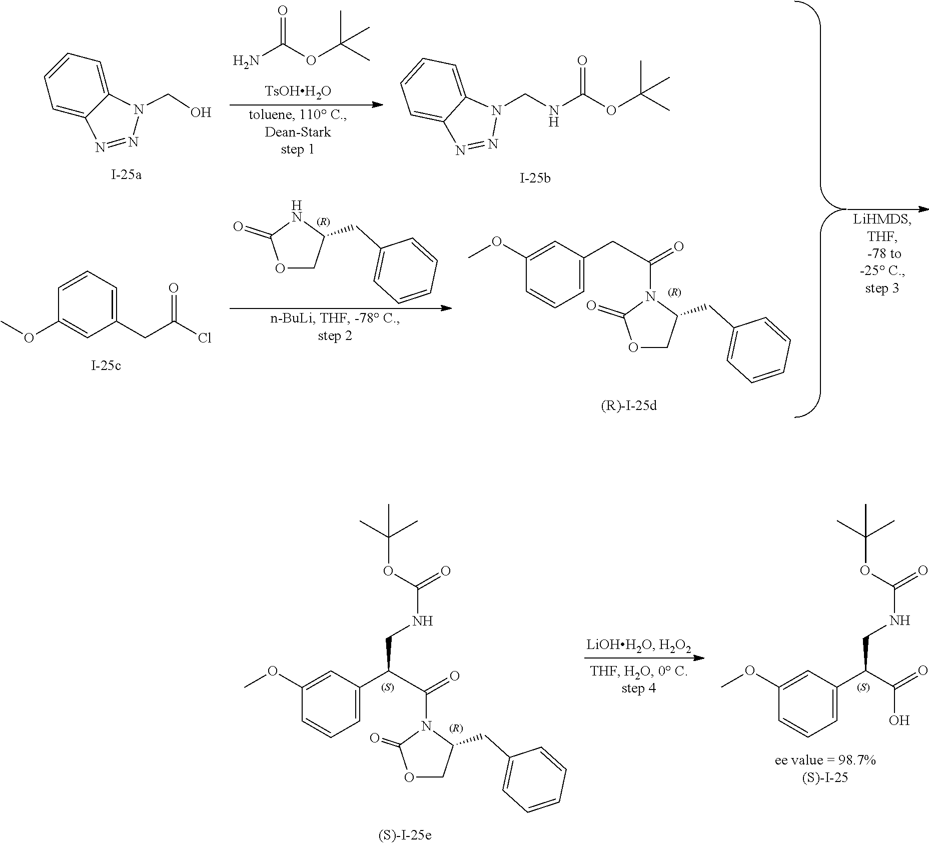

Step 1: To a solution of (1H-benzo[d][1,2,3]triazol-1-yl) methanol I-25a (25 g, 167.62 mmol) in toluene (400 mL) was added tert-butyl carbamate (19.64 g, 167.62 mmol) and 4-methylbenzenesulfonic acid hydrate (64 mg, 336.46 μmol, 51.61 μL) and the solution was refluxed at 120° C. using Dean-Stark trap for 24 h. Half of the toluene was evaporated under reduced pressure and the solution was cooled to 0° C. and the product was recrystallized to afford tert-butyl N-(benzotriazol-1-ylmethyl) carbamate I-25b (24 g, 58% yield) as white solid. LCMS: t_{R}=1.55 min in 3 min chromatography (3 min-5-95% MeCN in water (6 mmol/L NH₄HCO₃), Waters Acquity UPLC BEH C18 1.7 μm, 2.1*50 mm, 40° C.), MS (ESI) m/z=249.1 [M+H]⁺. ¹H NMR: (400 MHZ, DMSO-d₆) δ 8.42 (t, J=6.4 Hz, 1H), 8.04 (d, J=8.4 Hz, 1H), 7.96 (d, J=8.4 Hz, 1H), 7.57 (t, J=7.6 Hz, 1H), 7.44-7.38 (m, 1H), 5.88 (d, J=6.7 Hz, 2H), 1.37 (s, 9H).

Step 2: To (R)-4-benzyloxazolidin-2-one (9.1 g, 51.35 mmol) in THF (100 mL) at −78° C. was added 2.5 M of n-butyllithium (3.32 g, 51.87 mmol, 20.75 mL) slowly down the sides of the flask and the reaction was stirred at −78° C. for 20 min. Next, the solution containing the lithium anion of (R)-4-benzyloxazolidin-2-one in THF was cannulated into a precooled (−78° C.) solution of 2-(3-methoxyphenyl) acetyl chloride I-25c (10.05 g, 54.44 mmol) dissolved in THF (100 mL). The reaction was stirred at −78° C. for 1 h and then poured into NH₄Cl (sat.)/HCl (1 M) (3:1, v/v) and extracted with ethyl acetate. The combined extracts were dried over anhydrous sodium sulfate, filtered and evaporated under reduced pressure. The residue was purified by silica flash chromatography column, elution gradient from 0 to 40% ethyl acetate in petroleum ether. Pure fractions were lyophilized to dryness to afford (4R)-4-benzyl-3-[2-(3-methoxyphenyl) acetyl]oxazolidin-2-one I-25d (12.19 g, 73% yield) as white solid. LCMS: t_{R}=1.82 min in 3 min chromatography (3 min-5-95% MeCN in water (6 mmol/L NH₄HCO₃), Waters Acquity UPLC BEH C18 1.7 μm, 2.1*50 mm, 40° C.), MS (ESI) m/z=326.2 [M+H]⁺. ¹H NMR: (400 MHZ, DMSO-d₆) δ 7.32-7.20 (m, 4H), 7.19-7.08 (m, 2H), 6.86 (d, J=7.1 Hz, 3H), 4.68 (ddd, J=10.8, 7.6, 2.9 Hz, 1H), 4.34 (t, J=8.5 Hz, 1H), 4.25 (d, J=15.9 Hz, 1H), 4.20 (dd, J=8.9, 2.8 Hz, 1H), 4.10 (d, J=15.9 Hz, 1H), 3.75 (s, 3H), 2.95 (ddd, J=20.9, 13.5, 5.3 Hz, 2H).

Step 3: To a solution of 1M LiHMDS (10.68 g, 44.59 mmol) in THF (44.59 mL) cooled to −70° C.° C. was added a cooled (−70° C.) solution of (4R)-4-benzyl-3-[2-(3-methoxyphenyl) acetyl]oxazolidin-2-one I-25d (12.19 g, 37.47 mmol) in THF (60 mL) via cannula and this mixture was stirred at −70° C. for 40 min. Then a solution of tert-butyl N-(benzotriazol-1-ylmethyl) carbamate I-25b (11.16 g, 44.96 mmol) in THF (60 mL), also cooled to −70° C., was added via cannula. This solution was warmed to −25 to −30° C. and stirred for 2 h. The mixture was poured into ethyl acetate and NH₄Cl (sat.)/HCl (1 M) (3:1) and the aqueous phase was further extracted one more time with ethyl acetate. The combined organics were dried anhydrous sodium sulfate, filtered and evaporated under reduced pressure. The residue was purified by silica flash chromatography column, elution gradient from 0 to 4% ethyl acetate in DCM. Pure fractions were lyophilized to dryness to afford tert-butyl N-[(2S)-3-[(4R)-4-benzyl-2-oxo-oxazolidin-3-yl]-2-(3-methoxyphenyl)-3-oxo-propyl]carbamate I-25e (15 g, 88% yield) as a soft white solid. LCMS: t_{R}=1.95 min in 3 min chromatography (3 min-5-95% MeCN in water (0.02% TFA), Waters Acquity UPLC BEH C18 1.7 μm, 2.1*50 mm, 40° C.), MS (ESI) m/z=455.3 [M+H]⁺. ¹H NMR: (500 MHZ, DMSO-d₆) δ 7.37-7.17 (m, 6H), 7.08 (t, J=5.6 Hz, 1H), 6.83 (t, J=7.1 Hz, 2H), 6.78 (s, 1H), 5.16 (dd, J=8.4, 6.1 Hz, 1H), 4.68 (t, J=7.6 Hz, 1H), 4.25 (t, J=8.5 Hz, 1H), 4.17 (dd, J=12.4, 10.3 Hz, 1H), 3.72 (s, 3H), 3.67-3.57 (m, 1H), 3.29-3.17 (m, 1H), 3.02 (ddd, J=20.6, 13.5, 4.9 Hz, 2H), 1.35 (s, 9H).

Step 4: To tert-butyl N-[(2S)-3-[(4R)-4-benzyl-2-oxo-oxazolidin-3-yl]-2-(3-methoxyphenyl)-3-oxo-propyl]carbamate I-25e (15 g, 33.00 mmol) in THF (270 mL) and water (90 mL) cooled to 0° C. was added 30% of hydrogen peroxide (16.36 g, 144.26 mmol, 14.87 mL, 30% purity) and lithium hydroxide (1.03 g, 42.90 mmol). The solution was stirred at 0° C. for 0.75 h and 400 ml of saturated solution of sodium sulfite was added. The mixture was stirred for an additional 15 min, then acidified with 1 M of HCl until pH=5 (pH paper) and extracted with ethyl acetate (200 mL×3). The combined organics were dried with anhydrous sodium sulfate, filtered and evaporated under reduced pressure. The residue was purified by silica flash chromatography column, elution gradient from 0 to 40% ethyl acetate in petroleum ether with 1% acetic acid. Pure fractions were lyophilized to dryness to afford (2S)-3-(tert-butoxycarbonylamino)-2-(3-methoxyphenyl)propanoic acid I-25 (8.7 g, 89% yield) as white solid. ¹H NMR: (500 MHz, DMSO-d₆) δ 12.33 (s, 1H), 7.24 (t, J=7.9 Hz, 1H), 3.79-3.63 (m, 4H), 3.74-3.71 (m, 4H) 3.42 (ddd, J=13.3, 7.8, 5.4 Hz, 1H), 3.21 (dt, J=13.3, 6.7 Hz, 1H), 1.33 (s, 9H). SFC t_{R}=4.638 min, ee value=98.7%.

### Synthesis of Intermediate I-26

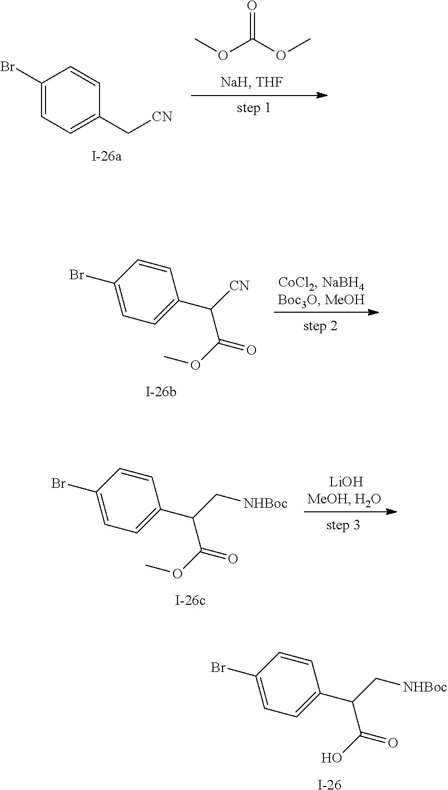

Step 1: To a stirred mixture of 4-bromo-benzeneacetonitrile I-26a (10 g, 51.01 mmol, l equiv) in THF (200 mL) was added sodium hydride (2.45 g, 102.02 mmol, 2 equiv) at 0° C. The resulting mixture was stirred at 0° C. for 2 hours under air atmosphere. Then dimethyl carbonate (18.38 g, 204.03 mmol, 4 equiv) was added to the above mixture. After reaction, the resulting mixture was quenched by ice water (200 mL). The resulting mixture was extracted with ethyl acetate (3×100 mL). The combined organic layers were dried over anhydrous Na₂SO₄. After filtration, the filtrate was concentrated under reduced pressure. The residue was purified by silica column chromatography, eluted with PE/EA (10:1, v/v) to afford methyl 2-(4-bromophenyl)-2-cyanoacetate I-26b (6.1 g, 47.07%) as a yellow oil. ¹H NMR (DMSO-d₆, 400 MHz) δ 3.74 (3H, s), 5.74 (1H, s), 7.41 (2H, m), 7.68 (2H, m).

Step 2: To a stirred mixture of nitrile I-26b (5.0 g, 19.68 mmol, 1 equiv) in methanol (35 mL) was added CoCl₂·6H₂O (1102.40 mg, 39.36 mmol, 2 equiv), (Boc)₂O (8589.67 mg, 39.36 mmol, 2 equiv) was added NaBH₄ (4466.65 mg, 118.07 mmol, 6 equiv) at 0° C. The resulting mixture was stirred at 25° C. for 4 hour under air atmosphere. After reaction, the resulting mixture was quenched by added ice water (100 mL). The resulting mixture was extracted with ethyl acetate (3×70 mL). The combined organic layers were dried over anhydrous Na₂SO₄. After filtration, the filtrate was concentrated under reduced pressure. The residue was purified by silica column chromatography, eluted with PE/EA (5:1, v/v) to afford methyl 2-(4-bromoph enyl)-3-[(tert-butoxycarbonyl)amino]propanoate I-26c (4.0 g, 56.74%) as a yellow oil. ¹H NMR (DMSO-d₆, 400 MHz) δ 1.34 (9H, d), 3.26 (1H, m), 3.48 (1H, m), 3.60 (3H, d), 3.85 (1H, t), 6.94 (1H, t), 7.24 (2H, m), 7.34 (1H, m), 7.53 (1H, m).

Step 3: To a stirred mixture of ester I-26c (4.0 g, 11.17 mmol, 1 equiv) in methanol (16 mL) was added LiOH·H₂O (1874.09 mg, 44.66 mmol, 4 equiv) in water (4 mL) The resulting mixture was stirred at 60° C. for 8 hour under nitrogen atmosphere. After cooled to 25° C., the resulting mixture was concentrated under reduced pressure. The reaction mixture was diluted with water (20 mL), and adjust pH to 2 with HCl (1 M, 30 mL). The residue was purified by reverse flash chromatography with the following conditions: column, C18 silica; mobile phase, phase A, water. Phase B ACN, from 0% to 50% gradient in 40 min; detector, UV 254 nm, and hold for 20 min at 30%. After evaporated the solvent, 2-(4-bromophenyl)-3-[(tert-butoxycarbonyl)amino]propanoic acid I-26 (3.2 g, 83.26%) was obtained as a yellow solid. LCMS: m/z (ESI), [M-Bu]⁺=289.95.

### Synthesis of Intermediate I-27

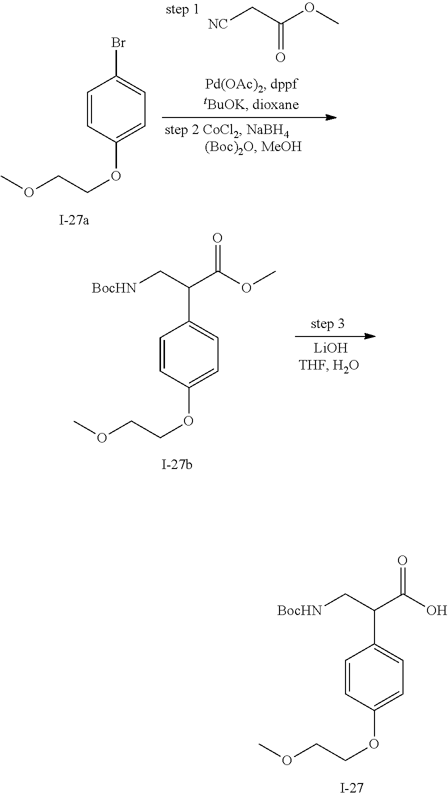

Step 1: To a stirred solution of 1-bromo-4-(2-methoxyethoxy)benzene I-27a (1 g, 4.35 mmol, 1 equiv) and methyl cyanoacetate (198.18 mg, 2.00 mmol, 0.46 equiv) and Pd(OAc)₂ (97.61 mg, 0.44 mmol, 0.1 equiv), t-BuOK (975.77 mg, 8.70 mmol, 2 equiv) in dioxane (15 mL) were added Dppf (480.33 mg, 0.87 mmol, 0.2 equiv) at room temperature under air atmosphere. The resulting mixture was stirred for overnight at 70° C. under nitrogen atmosphere. The resulting mixture was extracted with ethyl acetate (3×20 mL), dried over anhydrous Na₂SO₄. After filtration, the filtrate was concentrated under reduced pressure. The residue was purified by prep-TLC with PE/EA (1:1, v/v) to afford methyl 2-cyano-2-[4-(2-methoxyethoxy)phenyl]acetate (200 mg, 9.26%) as yellow oil. ¹H NMR (CDCl₃, 400 MHZ) δ 3.48 (3H, s), 3.76-3.80 (2H, m), 3.82 (3H, s), 4.12-4.17 (2H, m), 6.98 (2H, d), 7.39 (2H, d).

Step 2: To a stirred mixture of methyl 2-cyano-2-[4-(2-methoxyethoxy)phenyl]acetate (300 mg, 1.20 mmol, 1 equiv) and CoCl₂·6H₂O (572.69 mg, 2.41 mmol, 2 equiv) and (Boc)₂O (525.34 mg, 2.41 mmol, 2.00 equiv) in methanol (10 mL) was added NaBH₄ (364.24 mg, 9.63 mmol, 8 equiv) in portions at 0° C. under air atmosphere. The mixture in methanol was stirred for overnight at 0° C. under air atmosphere. The reaction was quenched with water (10 mL) at room temperature. The resulting mixture was extracted with ethyl acetate (3×15 mL), dried over anhydrous Na₂SO₄. After filtration, the filtrate was concentrated under reduced pressure. The residue was purified by prep-TLC with CH₂Cl₂/methanol (15:1, v/v) to afford methyl 3-[(tert-butoxycarbonyl)amino]-2-[4-(2-methoxyethoxy)phenyl]propanoate I-27b (100 mg, 23.51%) as a white solid. LCMS: m/z (ESI), [M+H−boc]⁺=254.

Step 3: To a stirred mixture of ester I-27b (200 mg, 0.57 mmol, 1 equiv) in THF (40 mL) and H₂O (10 mL) was added LiOH (40.66 mg, 1.70 mmol, 3 equiv) in portions at 0° C. under nitrogen atmosphere. The resulting mixture was stirred for overnight at 60° C. under nitrogen atmosphere. The mixture was allowed to cool down to room temperature. The residue was purified by reverse flash chromatography with the following conditions: column, C18 silica; mobile phase, MeCN in water (0.1% FA), 10% to 50% gradient in 10 min; detector, UV 254 nm. This resulted in 3-[(tert-butoxycarbonyl)amino]-2-[4-(2-methoxyethoxy)phenyl]propanoic acid I-27 (90 mg, 46.86%) as a white solid. LCMS: m/z (ESI), [M+H-boc]⁺=240.

### Synthesis of Intermediate I-28

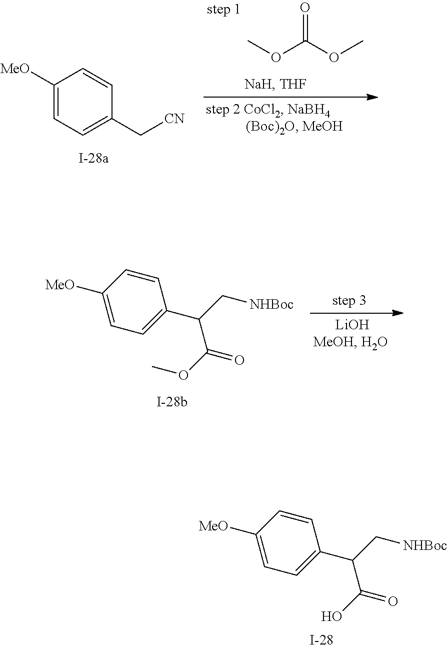

Step 1: To a solution of 2-(4-methoxyphenyl) acetonitrile I-28a (6 g, 40.77 mmol, 1 equiv) in THF (100 mL) was added sodium hydride (1.47 g, 61.15 mmol, 1.5 equiv) 60% in oil at 0° C. The mixture was stirred for 80 min. dimethyl carbonate (29.38 g, 326.14 mmol, 8 equiv) was added at 0° C. and the mixture was allowed to warm to RT and stirred for 4 h. The reaction mixture was quenched by water (100 mL) and extracted with DCM (3×100 mL). The residue was purified by silica column chromatography, eluted with PE/EA (8:1, v/v) to afford methyl 2-cyano-2-(4-methoxyphenyl)acetate (5.96 g, 70.03%) as a yellow oil.

Step 2: NaBH₄ (40.56 g, 107.21 mmol, 4 equiv) was added to a mixture of methyl 2-cyano-2-(4-methoxyphenyl)acetate (5.50 g, 26.80 mmol, 1 equiv), (Boc)₂O (11.7 g, 53.60 mmol, 2 equiv) and CoCl₂·6H₂O (8.29 mg, 34.84 mmol, 1.3 equiv) and in methanol (10 mL) portion wise, the mixture was stirred for overnight at room temperature under nitrogen atmosphere. The reaction was quenched with water (50 mL) at 0° C. The resulting mixture was extracted with DCM (3×40 mL). The combined organic layers were washed with saturated brine (1×100 mL), dried over anhydrous Na₂SO₄. After filtration, the filtrate was concentrated under reduced pressure. The residue was purified by silica column chromatography, eluted with PE/EA (5:1, v/v) to afford methyl 3-[(tert-butoxycarbonyl)amino]-2-(4-methoxyphenyl)propanoate I-28b (3.50 mg, 38.16%) as a colorless oil. LCMS: m/z (ESI), [M+H]⁺=310.15.

Step 3: Into a 50 mL round-bottom flask were added ester I-28b (2 g, 6.47 mmol, 1 equiv) and LiOH·H₂O (0.54 g, 12.93 mmol, 2 equiv) and H₂O (4 mL) and methanol (8 mL) at room temperature. The resulting mixture was stirred for 2 h at room temperature under air atmosphere. The residue was acidified to pH=5 with HCl (1M). The residue was purified by reverse flash chromatography with the following conditions: column, C18 silica; mobile phase, methanol in water, 0% to 15% gradient in 30 min; detector, UV 220 nm. The resulting liquid was dried by lyophilization. This resulted in 3-[(tert-butoxycarbonyl)amino]-2-(4-methoxyphenyl)propanoic acid I-28 (1.38 g, 70.83%) as a white solid. LCMS: m/z (ESI), [M+H−^{t}Bu]⁺=240.25.

### Synthesis of Intermediate I-29

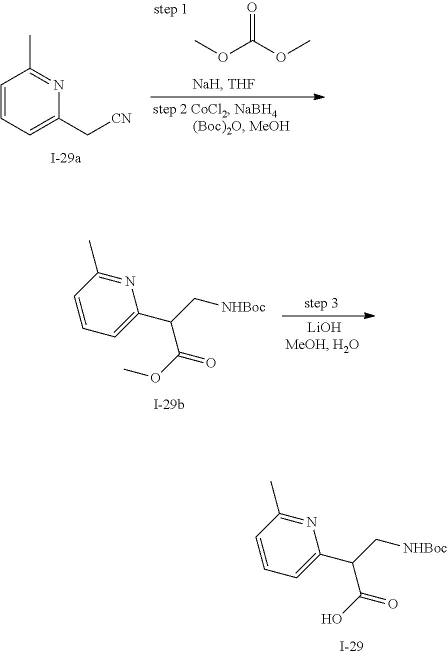

Step 1: To a stirred solution of 2-(6-methylpyridin-2-yl) acetonitrile I-29a (500 mg, 3.78 mmol, 1 equiv) in THF (20 mL) was added sodium hydride (181.57 mg, 7.57 mmol, 2 equiv) in portion at 0° C. under air atmosphere. The resulting mixture was stirred for 1 h at room temperature under air atmosphere. To the above mixture was added dimethylcarbonate (436.34 mg, 4.54 mmol, 1.2 equiv) at room temperature. The resulting mixture was stirred for additional 2 h at room temperature. The reaction was quenched by the addition of water/ice (20 mL) at room temperature. The resulting mixture was extracted with CH₂Cl₂ (3×20 mL). The combined organic layers were dried over anhydrous Na₂SO₄. After filtration, the filtrate was concentrated under reduced pressure. The residue was purified by prep-TLC with CH₂Cl₂/methanol (20:1, v/v) to afford methyl 2-cyano-2-(6-methylpyridin-2-yl)acetate (330 mg, 45.86%) as orange solid. LCMS: m/z (ESI), [M+H]⁺=191.20. ¹H NMR (CDCl₃, 400 MHz) δ 2.49 (3H, s), 3.82 (3H, s), 6.49 (1H, d), 7.18 (1H, d), 7.54 (1H, dd).

Step 2: To a stirred solution of methyl 2-cyano-2-(6-methylpyridin-2-yl)acetate (1.8 g, 9.46 mmol, 1 equiv) in methanol (20 mL) was added CoCl₂·6H₂O (6.75 g, 28.39 mmol, 3 equiv) and (Boc)₂O (6.20 g, 28.39 mmol, 3 equiv) dropwise at room temperature under air atmosphere. To the above mixture was added NaBH₄ (2.86 g, 75.71 mmol, 8 equiv) dropwise at 0° C. The resulting mixture was stirred for additional overnight at room temperature. The reaction was quenched by the addition of water (20 mL) at room temperature. The resulting mixture was extracted with CH₂Cl₂ (3×20 mL). The resulting mixture was concentrated under reduced pressure. The residue was purified by prep-TLC with CH₂Cl₂/methanol (50:1, v/v) to afford methyl 3-[(tert-butoxycarbonyl)amino]-2-(6-methylpyridin-2-yl)propanoate I-29b (1 g, 35.90%) as a yellow oil. ¹H NMR (DMSO-d₆, 400 MHz) δ 1.34 (9H, s), 2.43 (3H, s), 3.31-3.44 (1H, m), 3.50-3.60 (4H, m), 3.99 (1H, t), 6.87 (1H, t), 7.13 (2H, dd), 7.65 (1H, t).

Step 3: To a stirred solution of ester I-29b (1 g, 3.40 mmol, 1 equiv) in methanol (20 mL) and H₂O (10 mL) was added LiOH (0.16 g, 6.69 mmol, 1.97 equiv) at room temperature. The resulting mixture was stirred for 2 h at 60° C. under air atmosphere. The resulting mixture was concentrated under reduced pressure. The residue was purified by reverse flash chromatography with the following conditions: column, C18 silica; mobile phase, MeCN in water, 10% to 20% gradient in 10 min; detector, UV 254 nm. to afford 3-[(tert-butoxycarbonyl)amino]-2-(6-methylpyridin-2-yl)propanoic acid I-29 (865 mg, 90.83%) as a yellow solid. ¹H NMR (DMSO-d₆, 400 MHz) δ 1.34 (9H, s), 2.41 (3H, s), 3.34-3.38 (2H, m), 3.45 (1H, t), 6.51 (1H, t), 6.99 (1H, d), 7.09 (1H, d), 7.51 (1H, t).

### Synthesis of Intermediate I-30

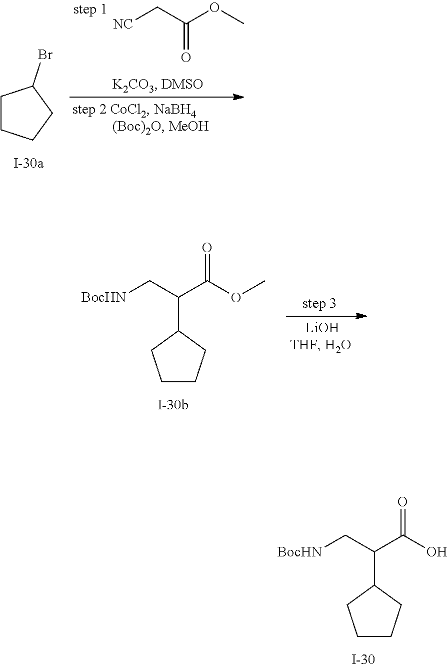

Step 1: To a stirred mixture of ethyl cyanoacetate (3 g, 26.52 mmol, 1 equiv) and bromocyclopentane I-30a (4.74 g, 31.83 mmol, 1.2 equiv) in DMSO (50 mL) was added K₂CO₃ (11.00 g, 79.56 mmol, 3 equiv) at room temperature under air atmosphere. The resulting mixture was stirred for 2 h at 60° C.° C. under air atmosphere. After cooled to room temperature, the reaction was quenched by the addition of water (20 mL) at room temperature. The resulting mixture was extracted with ethyl acetate (3×20 mL). The combined organic layers were washed with brine (3×20 mL), dried over anhydrous Na₂SO₄. After filtration, the filtrate was concentrated under reduced pressure. The residue was purified by reverse flash chromatography with the following conditions: column, C18 silica; mobile phase, MeCN in water, 40% to 50% gradient in 10 min; detector, UV 254 nm to afford ethyl 2-cyano-2-cyclopentylacetate (1.1 g, 22.89%) as a yellow oil. ¹H NMR (DMSO-d₆, 400 MHZ) δ 1.23 (3H, t), 1.28-1.41 (2H, m), 1.46-1.60 (2H, m), 1.60-1.71 (2H, m), 1.71-1.90 (2H, m), 2.40 (1H, ttd), 4.19-4.26 (2H, m).

Step 2: To a stirred mixture of ethyl 2-cyano-2-cyclopentylacetate (900 mg, 4.97 mmol, 1 equiv) and CoCl₂·6H₂O (3.54 g, 14.90 mmol, 3 equiv) in methanol (20 mL) was added (Boc) 20 (3.25 g, 14.90 mmol, 3 equiv) dropwise at room temperature under air atmosphere. To the above mixture was added NaBH₄ (1.50 g, 39.73 mmol, 8 equiv) in portion over 3 min at 0° C. The resulting mixture was stirred for overnight at room temperature under air atmosphere. The reaction was quenched by the addition of water/ice of (20 mL) at 0° C. and the mixture was concentrated under reduced pressure. The resulting mixture was extracted with ethyl acetate (3×20 mL). The combined organic layers were washed with brine (3×20 mL), dried over anhydrous Na₂SO₄. After filtration, the filtrate was concentrated under reduced pressure. The residue was purified by silica column chromatography, eluted with PE/EA (1:1, v/v) to afford ethyl 3-[(tert-butoxycarbonyl)amino]-2-cyclopentylpropanoate I-30b (900 mg, 63.51%) as a yellow oil. ¹H NMR (DMSO-d₆, 400 MHz) δ 1.18 (3H, s), 1.36 (9H, s), 1.50-1.67 (6H, m), 1.77-1.88 (2H, m), 2.35 (1H, td), 3.02-3.13 (2H, m), 3.13-3.19 (1H, m), 4.05 (2H, d), 6.86 (1H, t).

Step 3: To a stirred solution of ester I-30b (1.1 g, 3.85 mmol, 1 equiv) in methanol (10 mL) and H₂O (5 mL) was added LiOH (184.63 mg, 7.71 mmol, 2 equiv) at room temperature under air atmosphere. The resulting mixture was stirred for 2 h at 60° C.° C. under air atmosphere. After cooled to room temperature, the resulting mixture was concentrated under reduced pressure. The residue was purified by reverse flash chromatography with the following conditions: column, C18 silica; mobile phase, MeCN in water, 20% to 30% gradient in 10 min; detector, UV 254 nm to afford 3-[(tert-butoxycarbonyl)amino]-2-cyclopentylpropanoic acid I-30 (878 mg, 88.52%) as a white solid. ¹H NMR (DMSO-d₆, 400 MHz) δ 1.36 (9H, s), 1.57 (8H, d), 1.83 (1H, s), 3.00 (2H, s), 6.52 (1H, s).

### Synthesis of Intermediate I-31

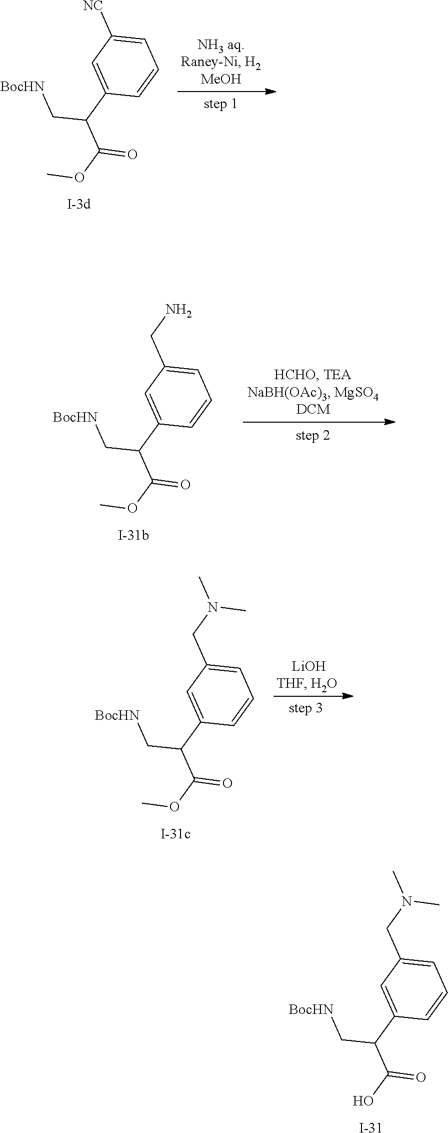

Step 1: Into a 50 mL round-bottom flask were added methyl 3-[(tert-butoxycarbonyl)amino]-2-(3-cyanophenyl)propanoate I-3d (300 mg, 0.99 mmol, 1 equiv), NH₃ aq (2.5 mL, 64.20 mmol, 65.13 equiv) and Raney Ni (10 mg, 0.12 mmol, 0.12 equiv) in methanol (10 mL) at room temperature. The resulting mixture was stirred for overnight at room temperature under H₂ atmosphere. The resulting mixture was filtered and the filtered cake was washed with methanol (2×20 mL). The filtrate was concentrated under reduced pressure, to afford methyl 2-[3-(aminomethyl)phenyl]-3-[(tert-butoxycarbonyl)amino]propanoate I-31b (260 mg, 77.94%) as a green oil. LCMS: m/z (ESI), [M+H]⁺=309.15.

Step 2: To a solution of amine I-31b (240 mg, 0.78 mmol, 1 equiv) and paraformaldehyde (1402.09 mg, 15.56 mmol, 20 equiv) in DCM (100 mL) were added triethylamine (157.51 mg, 1.56 mmol, 2 equiv) and MgSO₄ (93.67 mg, 0.78 mmol, 1 equiv) at 25° C. under N₂ atmosphere. The mixture was stirred for 30 minute at 25° C. And the resulting mixture was added NaBH(OAc)₃ (659.78 mg, 3.11 mmol, 4 equiv) at 25° C. The resulting mixture was stirred for 12 hours at 25° C. The resulting mixture was filtered and the filtered cake was washed with methanol (3×100 mL). The filtrate was concentrated under reduced pressure. The residue was purified by prep-TLC with PE/EA (3:1, v/v) to afford methyl 3-[(tert-butoxycarbonyl)amino]-2-{3-[(dimethylamino)methyl]phenyl}propanoate I-31c (115 mg, 37.24%) as a yellow oil. LCMS: m/z (ESI), [M+H]⁺=337.20.

Step 3: To a solution of ester I-31c (105 mg, 0.31 mmol, 1 equiv)) in THF (20 mL) and H₂O (4 mL) was added LiOH·H₂O (26.19 mg, 0.62 mmol, 2 equiv) at 25° C. under N₂ atmosphere. The mixture was stirred for 2 hours at 25° C. The resulting mixture was concentrated under reduced pressure. This resulted in 3-[(tert-butoxycarbonyl)amino]-2-{3-[(dimethylamino)methyl]phenyl}propanoic acid I-31 (100 mg, 69.15%) as a white solid. LCMS: m/z (ESI), [M+H]⁺=323.20.

### Synthesis of Intermediate I-32

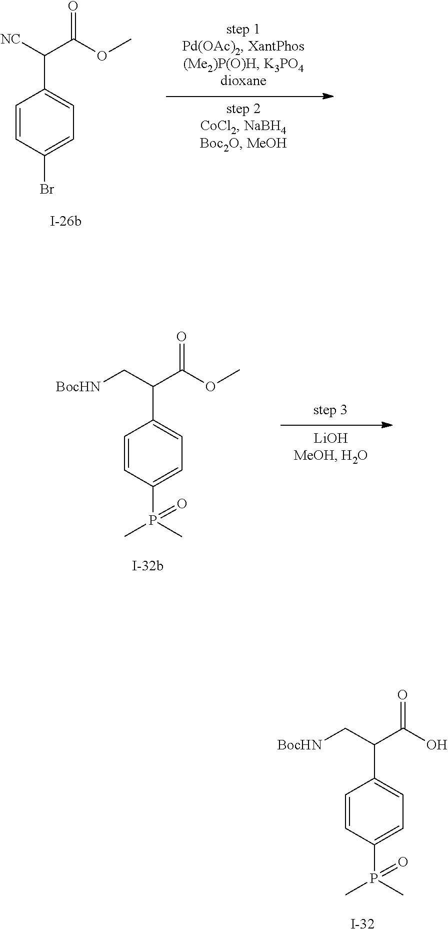

Step 1: To a stirred mixture of bromide I-26b (1.0 g, 3.94 mmol, 1 equiv) in 1,4-dioxane (10 mL) was added dimethylphosphine oxide (614.37 mg, 7.87 mmol, 2 equiv), Pd(OAc)₂ (176.72 mg, 0.79 mmol, 0.2 equiv), XantPhos (683.20 mg, 1.18 mmol, 0.3 equiv), and K₃PO₄ (2088.54 mg, 9.84 mmol, 2.5 equiv). The resulting mixture was stirred at 80° C. for 2 hr under nitrogen atmosphere. After cooled to 25° C., the resulting mixture was diluted with water (40 mL). The resulting mixture was extracted with ethyl acetate (3×50 mL). The combined organic layers were washed with water (3×30 mL), dried over anhydrous Na₂SO₄. After filtration, the filtrate was concentrated under reduced pressure. Then diluted with DMF (5 mL). The residue was purified by reverse flash chromatography with the following conditions: column, C18 silica; mobile phase, MeCN in water, 10% to 50% gradient in 10 min; detector, UV 220 nm, and hold for 15 min at 15%. After evaporated the solvent, methyl 2-cyano-2-[4-(dimethylphosphoryl)phenyl]acetate (220 mg, 22.25%) was obtained as a yellow solid. LCMS: m/z (ESI), [M+H]⁺=252.05.

Step 2: To a stirred mixture of methyl 2-cyano-2-[4-(dimethylphosphoryl)phenyl]acetate (660 mg, 2.63 mmol, 1 equiv), CoCl₂·6H₂O (1875.16 mg, 7.881 mmol, 3 equiv), (Boc)₂O (1146.75 mg, 5.25 mmol, 2 equiv) in MeOH (40 mL) was added NaBH₄ (795.08 mg, 21.02 mmol, 8 equiv) in portions at 0° C. under nitrogen atmosphere. The resulting mixture was stirred for overnight. After reaction, the reaction mixture was quenched by addition of ice water (40 mL). The resulting mixture was extracted with DCM (3×40 mL). The combined organic layers were dried over anhydrous Na₂SO₄. After filtration, the filtrate was concentrated under reduced pressure. The residue was purified by prep-TLC with PE/EA (5:1, v/v) to afford methyl 3-[(tert-butoxycarbonyl)amino]-2-[4-(dimethylphosphoryl)phenyl]propanoate I-32b (330 mg, 35.35%) as a yellow oil. LCMS: m/z (ESI), [M+H−^{t}Bu]⁺=300.10.

Step 3: To a stirred mixture of ester I-32b (310 mg, 0.87 mmol, 1 equiv) in methanol (4 mL) was added LiOH·H₂O (146.41 mg, 3.49 mmol, 4 equiv) in water (1 mL) dropwise at 25° C. under nitrogen atmosphere. The resulting mixture was stirred for overnight at 60° C. under nitrogen atmosphere. After cooled to 25° C., the resulting mixture was concentrated under reduced pressure and diluted with methanol (5 mL). The solution was acidified with HCl (1M, 10 mL). To the above solution was added DCM (10 mL). After extraction, the reaction mixture was evaporated under reduced pressure. The residue was diluted with DMF (2 mL) and purified by reverse flash chromatography with the following conditions: column, C18 silica; mobile phase, Phase B MeCN, Phase A water, from 10% to 50% gradient in 10 min; detector, UV 254 nm and hold for 20 min at 16.5%. After lyophilization, 3-[(tert-butoxycarbonyl)amino]-2-[4-(dimethylphosphoryl)phenyl]propanoic acid I-32 (200 mg, 67.17%) was obtained as a white solid. ¹H NMR (DMSO-d₆, 400 MHZ) δ 1.33 (9H, s), 1.60 (6H, d), 3.27 (2H, m), 7.37 (2H, dd), 7.60 (2H, m).

### Synthesis of Intermediate I-33

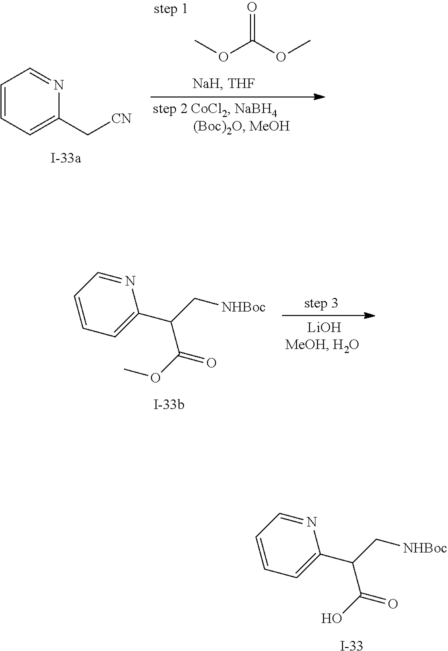

Step 1: To a stirred solution of 2-(pyridin-2-yl) acetonitrile I-33a (2.0 g, 16.93 mmol, 1.00 equiv) in THF (30 mL) was added sodium hydride (812.53 mg, 33.86 mmol, 2 equiv) at 0° C. The resulting mixture was stirred at 0° C. stirred for 30 min under nitrogen atmosphere. To the above mixture was added dimethyl carbonate (3049.90 mg, 33.86 mmol, 2 equiv) dropwise at 0° C. The resulting mixture was stirred for additional 3 hours at 25° C.° C. The reaction was quenched by the addition of water (80 mL) at 0° C. The resulting mixture was extracted with ethyl acetate (3×60 mL). The combined organic layers were washed with water (3×40 mL), dried over anhydrous Na₂SO₄. After filtration, the filtrate was concentrated under reduced pressure. The residue was purified by silica column chromatography, eluted with DCM/methanol (6%) to afford methyl 2-cyano-2-(pyridin-2-yl)acetate (2.3 g, 77.12%) as a yellow solid. LCMS: m/z (ESI), [M+H]⁺=176.95. ¹H NMR (DMSO-d₆, 400 MHz) δ 3.69 (3H, s), 6.88 (1H, t), 7.21 (1H, d), 7.84 (1H, ddd), 8.09 (1H, t).

Step 2: To a stirred mixture of methyl 2-cyano-2-(pyridin-2-yl)acetate (2.25 g, 12.77 mmol, 1 equiv), CoCl₂·6H₂O (6077.14 mg, 25.54 mmol, 2 equiv) and (Boc)₂O (5574.69 mg, 25.54 mmol, 2 equiv) in methanol (50 mL) was added NaBH₄ (2898.85 mg, 76.63 mmol, 6 equiv) at 0° C. The resulting mixture was stirred at 25° C. for 3 hour under air atmosphere. The reaction was quenched by the addition of water (80 mL) at 0° C. After filtration, the resulting mixture was extracted with DCM (60 mL×3). The combined organic layers were washed with water (3×50 mL), dried over anhydrous Na₂SO₄. After filtration, the filtrate was concentrated under reduced pressure. The residue was purified by prep-TLC with PE/EA (3/1, v/v) to afford methyl 3-[(tert-butoxycarbonyl)amino]-2-(pyridin-2-yl)propanoate I-33b (600 mg, 16.76%) as a yellow solid. LCMS: m/z (ESI), [M+H]⁺=281.05. ¹H NMR (DMSO-d₆, 400 MHZ) δ 1.34 (9H, s), 3.33 (1H, s), 3.42 (1H, dt), 3.55 (3H, dd), 4.04 (1H, q), 6.87 (1H, t), 7.30 (2H, m), 7.77 (1H, td), 8.51 (1H, m).

Step 3: To a stirred mixture of ester I-33b (575 mg, 2.05 mmol, 1 equiv) in methanol (4.0 mL) was added LiOH (196.50 mg, 8.20 mmol, 4 equiv) in water (1.0 mL) at 25° C. The resulting mixture was stirred at 70° C. for 3 hour under air atmosphere. After cooled to room temperature, the resulting mixture was concentrated under vacuum and diluted with methanol (5 mL). The solution was acidified with HCl (1M, 10 mL). To the above solution was added DCM (10 mL). After extraction, the reaction mixture was evaporated under reduced pressure. The residue was diluted with DMF (2 mL) The residue was purified by reverse flash chromatography with the following conditions: column, C18 silica; mobile phase, Phase B MeCN, Phase A water, 10% to 50% gradient in 10 min; detector, UV 254 nm and hold 20 min at 10.8%. After lyophilization to afford 3-[(tert-butoxycarbonyl)amino]-2-(pyridin-2-yl)propanoic acid I-33 (387 mg, 70.85%) as a white solid. LCMS: m/z (ESI), [M+H]⁺=267.00. ¹H NMR (DMSO-d₆, 400 MHZ) δ 1.33 (9H, s), 3.39 (1H, d), 3.43 (1H, m), 3.54 (1H, t), 6.50 (1H, t), 7.15 (1H, m), 7.32 (1H, dd), 7.63 (1H, td), 8.40 (1H, m).

### Synthesis of Intermediate I-34

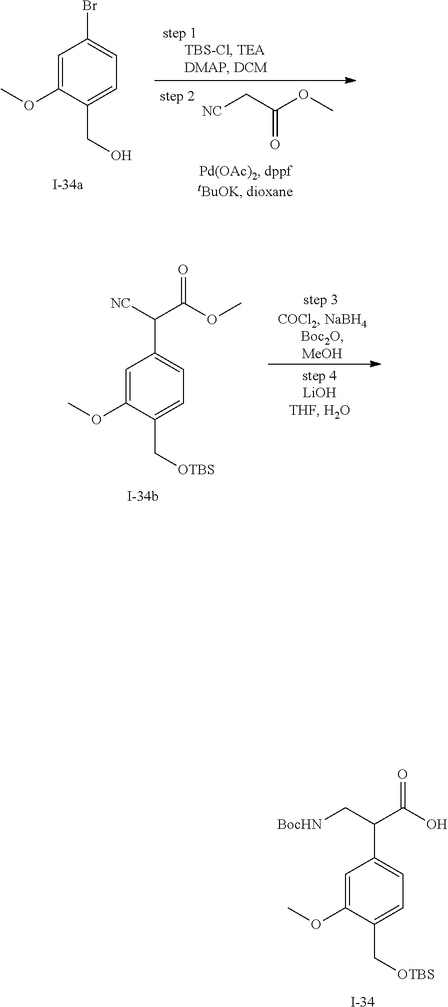

Step 1: A solution of (4-bromo-2-methoxyphenyl) methanol I-34a (2 g, 9.21 mmol, 1 equiv) in CH₂Cl₂ was treated with triethylamine (2.80 g, 27.64 mmol, 3 equiv) and DMAP (11.26 mg, 0.092 mmol, 0.01 equiv) for 5 min at 0° C. under air atmosphere followed by the addition of TBSCl (1.67 g, 11.06 mmol, 1.2 equiv) dropwise at 0° C. The resulting mixture was stirred for 1 h at room temperature under air atmosphere. The resulting mixture was diluted with water (100 mL). The aqueous layer was extracted with CH₂Cl₂ (3×100 mL). The combined organic layer was concentrated under reduced pressure. The residue was purified by prep-TLC with PE/EA (40:1, v/v) to afford [(4-bromo-2-methoxyphenyl) methoxy](tert-butyl)dimethylsilane (2.56 g, 83.86%) as a yellow oil. ¹H NMR (CD₃OD, 400 MHZ) δ 0.12 (6H, s), 0.97 (9H, s), 3.84 (3H, s), 4.70 (2H, d), 7.06-7.14 (2H, m), 7.31 (1H, dt).

Step 2: Into a 40 mL vial were added [(4-bromo-2-methoxyphenyl) methoxy](tert-butyl)dimethylsilane (1 g, 3.02 mmol, 1 equiv) and methyl cyanoacetate (358.88 mg, 3.62 mmol, 1.20 equiv) and Pd(OAc)₂ (67.76 mg, 0.30 mmol, 0.1 equiv) and dppf (333.43 mg, 0.60 mmol, 0.2 equiv) and t-BuOK (1.02 g, 9.05 mmol, 3 equiv) in 1,4-dioxane (8 mL) at room temperature. The resulting mixture was stirred for overnight at 90° C. under nitrogen atmosphere. The mixture was acidified to pH 5 with saturated NH₄Cl (aq.). The resulting mixture was stirred for 30 min at room temperature. The aqueous layer was extracted with ethyl acetate (3×100 mL). The combined organic layer was dried over anhydrous Na₂SO₄, filtered and concentrated under reduced pressure. The residue was purified by prep-TLC with PE/EA (4:1, v/v) to afford methyl 2-(4-{[(tert-butyldimethylsilyl)oxy]methyl}-3-methoxyphenyl)-2-cyanoacetate I-34b (350 mg, 33.18%) as a yellow oil. ¹H NMR (DMSO-d₆, 400 MHz) δ 0.09 (6H, d), 0.92 (9H, d), 3.74 (3H, d), 3.81 (3H, d), 4.68 (2H, s), 5.62 (1H, s), 6.99-7.05 (2H, m), 7.42 (1H, dd).

Step 3: Into a 40 mL vial were added nitrile I-34b (550 mg, 1.57 mmol, 1 equiv), CoCl₂·6H₂O (973.46 mg, 4.09 mmol, 2.6 equiv), and Boc₂O (1.37 g, 6.30 mmol, 4 equiv) in methanol (5 mL) at 0° C. NaBH₄ (476.25 mg, 12.59 mmol, 8 equiv) was added to the above mixture at 0° C. The resulting mixture was stirred for 2 h at room temperature. The reaction was quenched with water at room temperature. The resulting mixture was filtered and the filter cake was washed with DCM (3×30 mL). The filtrate was extracted with DCM (3×30 mL). The combined organic layers were dried over anhydrous Na₂SO₄, filtered and concentrated under reduced pressure. The residue was purified by prep-TLC with CH₂Cl₂/methanol (40:1, v/v) to afford methyl 3-[(tert-butoxycarbonyl)amino]-2-(4-{[(tert-butyldimethylsilyl)oxy]methyl}-3-methoxyphenyl)propanoate (410 mg, 57.43%) as a yellow oil. LCMS: m/z (ESI), [M+Na]⁺=476.15. ¹H NMR (DMSO-d₆, 400 MHZ) δ 0.08 (6H, s), 0.91 (9H, s), 1.34 (9H, s), 3.3 (1H, m), 3.41-3.54 (1H, m), 3.60 (3H, s), 3.78 (3H, s), 3.85 (1H, t), 4.59-4.67 (2H, m), 6.83 (2H, d), 6.94 (1H, t), 7.29 (1H, d).

Step 4: Into a 50 mL round-bottom flask were added methyl 3-[(tert-butoxycarbonyl)amino]-2-(4-{[(tert-butyldimethylsilyl)oxy]methyl}-3-methoxyphenyl)propanoate (390 mg, 0.86 mmol, 1 equiv), THF (2 mL, 24.69 mmol, 28.71 equiv) and LiOH·H₂O (72.15 mg, 1.72 mmol, 2.0 equiv) in water (1 mL) at room temperature. The resulting mixture was stirred for 1.5 h at room temperature. The residue was purified by reverse flash chromatography with the following conditions: column, C18 silica; mobile phase, methanol in water 0% to 100% gradient in 30 min; detector, UV 220 nm. The resulting mixture was concentrated under vacuum to afford 3-[(tert-butoxycarbonyl)amino]-2-(4-{[(tert-butyldimethylsilyl)oxy]methyl}-3-methoxyphenyl)propanoic acid I-34 (160 mg, 42.33%) as a grey solid. LCMS: m/z (ESI), [M+Na]⁺=462.15.

### Synthesis of Intermediate I-35

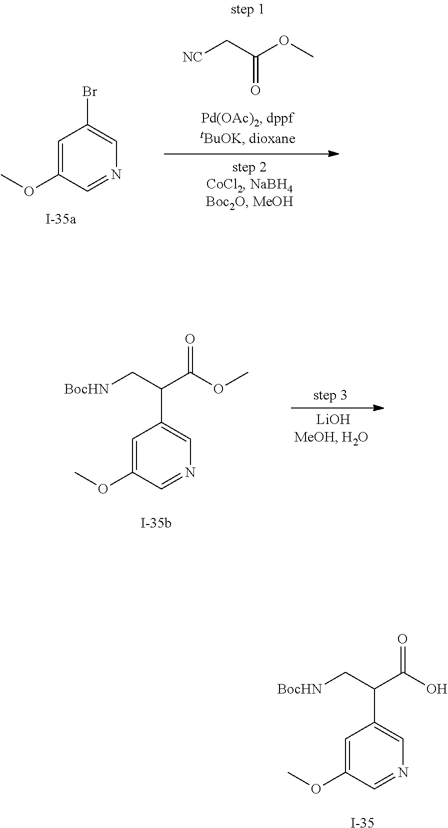

Step 1: Into a 100 mL round-bottom flask were added 3-bromo-5-methoxypyridine I-35a (4 g, 21.27 mmol, 1 equiv) and methyl cyanoacetate (2.53 g, 25.53 mmol, 1.2 equiv), Pd(OAc)₂ (477.62 mg, 2.13 mmol, 0.1 equiv), dppf (2.35 g, 4.26 mmol, 0.2 equiv), t-BuOK (7.16 g, 63.82 mmol, 3 equiv) in dioxane (40 mL, 590.14 mmol, 27.74 equiv) at room temperature. The resulting mixture was stirred for 5 h at 70° C. under nitrogen atmosphere. The mixture was acidified to pH 6 with saturated NH₄Cl (aq.). The resulting mixture was extracted with ethyl acetate (3×60 mL). The combined organic layers were washed with DCM (3×3 mL), dried over anhydrous Na₂SO₄. After filtration, the filtrate was concentrated under reduced pressure. The residue was purified by silica column chromatography, eluted with CH₂Cl₂/methanol (50:1, v/v) to afford methyl 2-cyano-2-(5-methoxypyridin-3-yl)acetate (3.8 g, 86.63%) as a light yellow solid. LCMS: m/z (ESI), [M+H]⁺=207.00.

Step 2: To a stirred mixture of methyl 2-cyano-2-(5-methoxypyridin-3-yl)acetate (700 mg, 3.40 mmol, 1 equiv) and CoCl₂·6H₂O (1.21 g, 5.09 mmol, 1.5 equiv) in methanol (15 mL) was added NaBH₄ (385.27 mg, 10.19 mmol, 3 equiv) in portions at 0° C. under nitrogen atmosphere. The resulting mixture was stirred for 1 h at room temperature under nitrogen atmosphere. The reaction was quenched with water at room temperature. The resulting mixture was filtered and the filter cake was washed with methanol (1×10 mL). The resulting mixture was extracted with DCM (3×100 mL). The combined organic layers were washed with DCM (2×3 mL), dried over anhydrous Na₂SO₄. After filtration, the filtrate was concentrated under reduced pressure. The residue was purified by prep-TLC with CH₂Cl₂/methanol (35:1, v/v) to afford methyl 3-[(tert-butoxycarbonyl)amino]-2-(5-methoxypyridin-3-yl)propanoate I-35b (250 mg, 23.73%) as a light yellow solid. LCMS: m/z (ESI), [M+H]⁺=311.15.

Step 3: Into a 50 mL round-bottom flask were added ester I-35b (80 mg, 0.26 mmol, 1 equiv) and LiOH (12.35 mg, 0.52 mmol, 2 equiv) in water (3 mL) and methanol (3 mL) at room temperature. The resulting mixture was stirred for 2 h at room temperature under nitrogen atmosphere. The mixture was acidified to pH 5 with conc. HCl. The resulting mixture was concentrated under reduced pressure. The residue was purified by reverse flash chromatography with the following conditions: column, C18 silica; mobile phase, methanol in water, 28% to 32% gradient in 15 min; detector, UV 254 nm. The resulting mixture was concentrated under vacuum. This resulted in 3-[(tert-butoxycarbonyl)amino]-2-(5-methoxypyridin-3-yl)propanoic acid I-35 (40 mg, 52.37%) as an off-white solid. LCMS: m/z (ESI), [M+H]⁺=297.05.

### Synthesis of Intermediate I-36

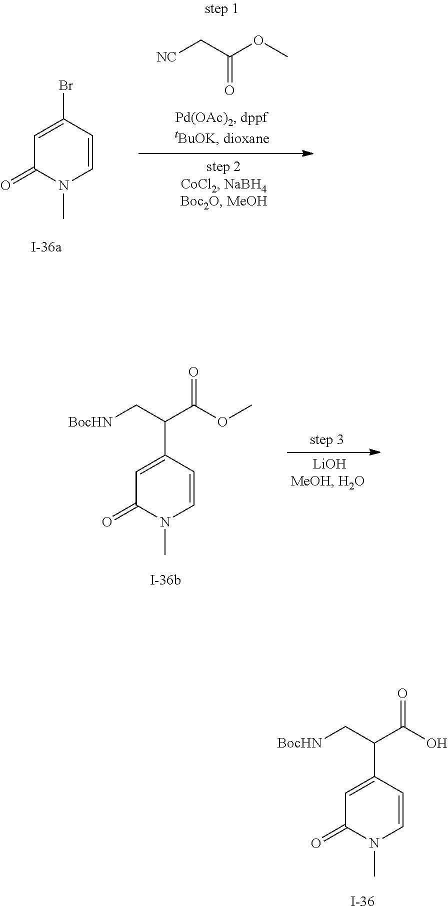

Step 1: Into a 250 mL round-bottom flask were added 4-bromo-1-methylpyridin-2-one I-36a (3 g, 15.96 mmol, 1 equiv) and methyl cyanoacetate (1.90 g, 19.15 mmol, 1.2 equiv) and Pd(OAc)₂ (358.21 mg, 1.60 mmol, 0.1 equiv) and Dppf (1.76 g, 3.19 mmol, 0.2 equiv) and t-BuOK (5.37 g, 47.87 mmol, 3 equiv) and 1,4-dioxane (100 mL) at room temperature. The resulting mixture was stirred for 1 h at 70° C. under nitrogen atmosphere. The reaction was quenched with water at room temperature. The mixture was acidified to pH 5 with HCl (1M). The residue was diluted with water (100 mL). The resulting mixture was washed with 3×100 mL of ethyl acetate. The resulting mixture was concentrated under reduced pressure. The residue was dissolved in methanol (5 mL). The residue was purified by reverse flash chromatography with the following conditions: column, C18 silica; mobile phase, methanol in water, 0% to 0% gradient in 15 min; detector, UV 254 nm. The resulting mixture was concentrated under reduced pressure. This resulted in methyl 2-cyano-2-(1-methyl-2-oxopyridin-4-yl)acetate (1.15 g, 34.95%) as a brown solid. LCMS: m/z (ESI), [M+H]⁺=207.05. ¹H NMR (CD₃OD, 400 MHZ) δ 3.37 (1H, s), 3.45 (3H, s), 3.63 (3H, s), 6.75 (1H, s), 6.91 (1H, s), 7.23 (1H, d).

Step 2: A solution of methyl 2-cyano-2-(1-methyl-2-oxopyridin-4-yl)acetate (1.1 g, 5.34 mmol, 1 equiv) in methanol (15 mL) was treated with Boc₂O (4.66 g, 21.34 mmol, 4 equiv) and CoCl₂·6H₂O (3.30 g, 13.87 mmol, 2.6 equiv) for 5 min at 0° C. under air atmosphere followed by the addition of NaBH₄ (1.61 g, 42.68 mmol, 8 equiv) in portions at 0° C. The resulting mixture was stirred for 15 h at 70° C. under air atmosphere. The reaction was quenched by the addition of water (80 mL) at room temperature. The resulting mixture was filtered and the filter cake was washed with DCM (3×10 mL). The aqueous layer was extracted with CH₂Cl₂ (3×80 mL). The resulting mixture was concentrated under reduced pressure. The residue was purified by prep-TLC with CH₂Cl₂/methanol (18:1, v/v) to afford methyl 3-[(tert-butoxycarbonyl)amino]-2-(1-methyl-2-oxopyridin-4-yl)propanoate I-36b (445 mg, 26.88%) as a white oil. LCMS: m/z (ESI), [M+H]⁺=311.15.

Step 3: Into a 100 mL round-bottom flask were added ester I-36b (445 mg, 1.43 mmol, 1 equiv) and LiOH·H₂O (120.33 mg, 2.87 mmol, 2 equiv) and THF (3 mL) and H₂O (3 mL) at room temperature. The resulting mixture was stirred for 2 h at room temperature under air atmosphere. The mixture was acidified to pH 5 with HCl (1M). The resulting mixture was concentrated under reduced pressure. The residue was dissolved in methanol (5 mL). The residue was purified by reverse flash chromatography with the following conditions: column, C18 silica; mobile phase, methanol in water, 0% to 10% gradient in 30 min; detector, UV 220 nm. The resulting mixture was concentrated under reduced pressure. The resulting liquid was dried by lyophilization. This resulted in 3-[(tert-butoxycarbonyl)amino]-2-(1-methyl-2-oxopyridin-4-yl)propanoic acid I-36 (350 mg, 82.37%) as an off-white solid. LCMS: m/z (ESI), [M+H]⁺=297.15. ¹H NMR (CD₃OD, 400 MHz) δ 1.43 (9H, d), 3.44 (2H, m), 3.56 (3H, d), 3.69 (1H, d), 6.43 (1H, d), 6.50 (1H, s), 7.63 (1H, d).

### Synthesis of Intermediate I-37

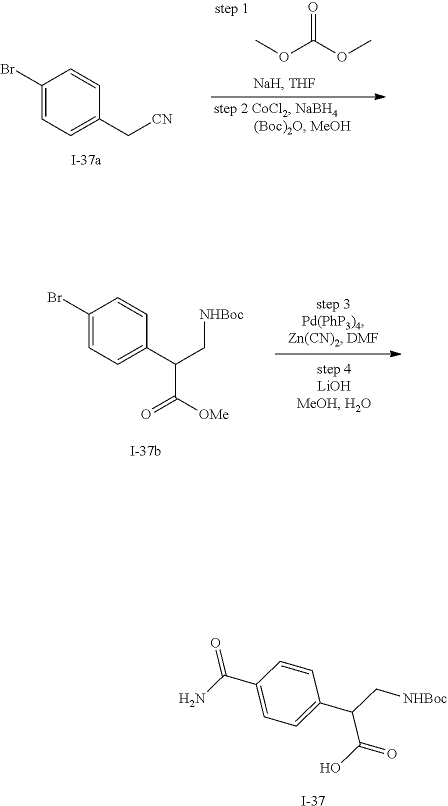

Step 1: To a stirred solution of 4-bromo-benzeneacetonitrile I-37a (10 g, 51.01 mmol, 1 equiv) in THF (120 mL) was added sodium hydride (4 g, 166.68 mmol, 3.27 equiv) at 0° C. The resulting mixture was stirred at 0° C. for 1.5 hour under air atmosphere. Then dimethyl carbonate (18.38 g, 204.03 mmol, 4 equiv) was added to the above mixture at 0° C. After reaction, the resulting mixture was diluted with water (20 mL). The resulting mixture was extracted with ethyl acetate (3×30 mL). The combined organic layers were dried over anhydrous Na₂SO₄. After filtration, the filtrate was concentrated under reduced pressure. The residue was purified by prep-TLC with PE/EA (2:1, v/v) to afford methyl 2-(4-bromophenyl)-2-cyanoacetate (8.923 g, 68.85%) as a yellow oil. ¹H NMR (CDCl₃, 400 MHz) δ 3.84 (3H, s), 4.72 (1H, s), 7.35-7.39 (2H, m), 7.56-7.62 (2H, m).

Step 2: Into a 250 mL round-bottom flask were added methyl 2-(4-bromophenyl)-2-cyanoacetate (2 g, 7.87 mmol, 1 equiv), Boc₂O (3.44 g, 15.74 mmol, 2 equiv) and CoCl₂·6H₂O (3.75 g, 15.74 mmol, 2 equiv) in methanol (70 mL) at room temperature. NaBH₄ (1.79 g, 47.23 mmol, 6 equiv) was added to the above mixture at 0° C. The resulting mixture was stirred for 2 h at 0° C. The resulting mixture was filtered and the filtered cake was washed with DCM (4×50 mL). The filtrate was extracted with CH₂Cl₂ (3×100 mL). The combined organic layers were concentrated under reduced pressure. The residue was purified by silica column chromatography, eluted with PE/EA with (1:1, v/v) to afford methyl 2-(4-bromophenyl)-3-[(tert-butoxycarbonyl)amino]propanoate I-37b (1.9 g, 67.38%) as a yellow oil. LCMS: m/z (ESI), [M+H−^{t}Bu]⁺=301.9/303.90. ¹H NMR (DMSO-d₆, 400 MHz) δ 1.33 (9H, s), 3.26 (1H, dt), 3.42-3.53 (1H, m), 3.60 (3H, s), 3.85 (1H, t), 7.19-7.25 (2H, m), 7.51-7.57 (2H, m).

Step 3: Into a 40 mL vial were added ester I-37b (1.9 g, 5.30 mmol, 1 equiv), Zn(CN)₂ (0.62 g, 5.30 mmol, 1.0 equiv) and Pd(PPh₃)₄ (0.61 g, 0.53 mmol, 0.1 equiv) in DMF (10 mL) at room temperature. The resulting mixture was stirred for 3 h at 80° C. under nitrogen atmosphere. The residue was washed with saturated NaHCO₃ (1×40 mL). The resulting mixture was extracted with ethyl acetate (3×200 mL). The combined organic layers were concentrated under reduced pressure. The residue was purified by prep-TLC with PE/EA (1:1, v/v) to afford methyl 3-[(tert-butoxycarbonyl)amino]-2-(4-cyanophenyl)propanoate (1.0 g, 61.95%) as a yellow solid. LCMS: m/z (ESI), [M+H−^{t}Bu]⁺=248.95. ¹H NMR (DMSO-d₆, 400 MHZ) δ 1.31 (9H, s), 3.51 (1H, ddd), 3.62 (3H, s), 4.01 (2H, dt), 7.44-7.50 (2H, m), 7.82 (2H, d).

Step 4: Into a 20 mL vial were added methyl 3-[(tert-butoxycarbonyl)amino]-2-(4-cyanophenyl)propanoate (900 mg, 2.96 mmol, 1 equiv), methanol (3.75 mL) and LiOH·H₂O (248.16 mg, 5.91 mmol, 2 equiv) in water (2.5 mL) at room temperature. The resulting mixture was stirred for 1.5 h at 70° C. The mixture was acidified to pH 5 with HCl (aq.). The residue was purified by reverse flash chromatography with the following conditions: column, C18 silica; mobile phase, methanol in water, 0% to 100% gradient in 30 min; detector, UV 254 nm. The resulting mixture was concentrated under vacuum, to afford 3-[(tert-butoxycarbonyl)amino]-2-(4-carbamoylphenyl)propanoic acid I-37 (800 mg, 87.74%) as a white solid. LCMS: m/z (ESI), [M+H−^{t}Bu]⁺=253.00.

### Synthesis of Intermediate I-38

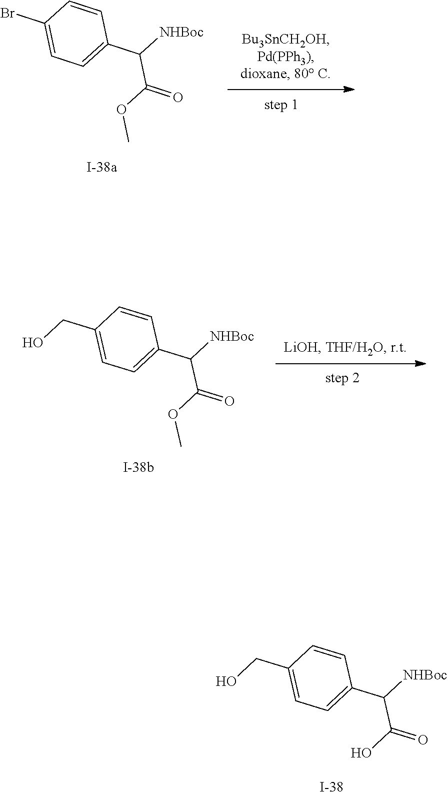

Step 1: To a solution of methyl 2-(4-bromophenyl)-2-[(tert-butoxycarbonyl)amino]acetate I-38a (500 mg, 1.45 mmol, 1 equiv) in dioxane (30 mL) were added (Bu)₃SnCH₂OH (700 mg, 2.18 mmol, 1.5 equiv) and Pd(PPh₃)₄ (84 mg, 0.073 mmol, 0.05 equiv) for 16 h at 80° C. under air atmosphere. The mixture was cooled to room temperature. The resulting mixture was quenched by water and extracted with ethyl acetate (3×30 mL). The combined organic layers were washed with sat. NaCl (3×30 mL), dried over anhydrous Na₂SO₄. After filtration, the filtrate was concentrated under reduced pressure. The residue was purified by prep-TLC eluted with petroleum ether/ethyl acetate (3:1, v/v) to give 325 mg of methyl 2-[(tert-butoxycarbonyl)amino]-2-[4-(hydroxymethyl)phenyl]acetate I-38b as an orange oil (76%). LCMS: m/z (ESI), [M+H]⁺=296.14. ¹H NMR (DMSO-d₆, 400 MHz) δ 1.39 (9H, s), 3.62 (3H, s), 5.22-5.26 (1H, d), 7.35-7.38 (2H, m), 7.52-7.59 (2H, m), 7.83-7.86 (1H, d).

Step 2: A mixture of methyl 2-[(tert-butoxycarbonyl)amino]-2-[4-(hydroxymethyl)phenyl]acetate (390 mg, 1.30 mmol, 1 equiv) and LiOH·H₂O (109 mg, 2.60 mmol, 2 equiv) in THF (4 mL) and H₂O (1 mL) for 5 h at room temperature under nitrogen atmosphere. The resulting mixture was concentrated under reduced pressure. The residue was purified by prep-HPLC with the following conditions (The mobile phase consisted of a mixture of solvent 0.1% NH₄HCO₃ in water and 0.1% NH₄OH in acetonitrile. A constant gradient from 95% aqueous/5% organic to 5% aqueous/95% organic mobile phase over the course of 25 minutes was utilized. The flow rate was constant at 40 mL/min.) to give 312 mg of [(tert-butoxycarbonyl)amino][4-(hydroxymethyl)phenyl]acetic acid I-38 (84%) as a yellow solid. LCMS: m/z (ESI), [M+H]⁺=282.13.

### Synthesis of Intermediate I-39

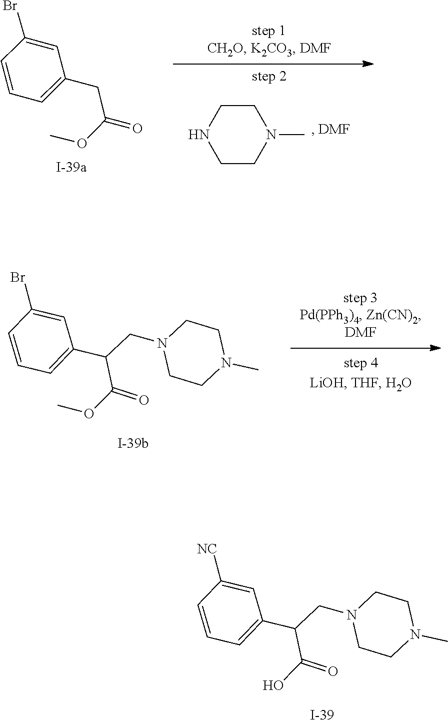

Step 1: A mixture of methyl 2-(3-bromophenyl)acetate I-39a (3.0 g, 13.10 mmol, 1 equiv) in DMF (4.0 mL) was added K₂CO₃ (2171.96 mg, 15.72 mmol, 1.20 equiv), polyoxymethylene (601.93 mg, 13.36 mmol, 1.02 equiv). The resulting mixture was stirred for 2 h at 70° C. under nitrogen atmosphere. After cooling to room temperature, the resulting mixture was diluted with water (40 mL). The resulting mixture was extracted with ethyl acetate (3×40 mL). The combined organic layers were washed with water (3×10 mL), dried over anhydrous Na₂SO₄. After filtration, the filtrate was concentrated under reduced pressure. The residue was purified by prep-TLC with PE/EA (3:1, v/v) to afford methyl 2-(3-bromophenyl)prop-2-enoate (850 mg, 26.92%) as a yellow oil. ¹H NMR (DMSO-d₆, 400 MHz) δ 3.76 (3H, s), 6.12 (1H, d), 6.33 (1H, d), 7.35 (1H, t), 7.44 (1H, ddd), 7.57 (1H, ddd), 7.64 (1H, t).

Step 2: A mixture of methyl 2-(3-bromophenyl)prop-2-enoate (800 mg, 3.32 mmol, 1 equiv) in DMF (5 mL) was added 1-methylpiperazine (1661.86 mg, 16.59 mmol, 5 equiv). The resulting mixture was stirred for 2 h at 25° C. under nitrogen atmosphere. The reaction mixture was diluted with EA (20 mL). The residue was washed with saturated NaHCO₃ (2×10 mL), then water (2×20 mL), and saturated NaCl (3×20 mL). The resulting organic layer was dried with Na₂SO₄. The resulting mixture was concentrated under reduced pressure. The residue was purified by reverse flash chromatography with the following conditions: column, C18 silica; mobile phase, MeCN in water, 10% to 50% gradient in 10 min; detector, UV 254 nm, hold for 30 min at 65%. After evaporated the water, methyl 2-(3-bromophenyl)-3-(4-methylpiperazin-1-yl)propanoate I-39b (740 mg, 65.35%) was obtained as a yellow oil. LCMS: m/z (ESI), [M+H]⁺=341.00, 343.00.

Step 3: To a stirred mixture of methylpiperazine I-39b (740 mg, 1.32 mmol, 1 equiv) and Zn(CN)₂ (154.84 mg, 1.32 mmol, 1.0 equiv) in DMF (3.0 mL) was added Pd(PPh₃)₄ (152.39 mg, 0.13 mmol, 0.1 equiv) in portions at room temperature under nitrogen atmosphere. The resulting mixture was stirred for 2.0 h at 80° C. under nitrogen atmosphere. The mixture was allowed to cool down to room temperature. The resulting mixture was diluted with water (20 mL). The resulting mixture was extracted with ethyl acetate (3×30 mL). The combined organic layers were washed with water (3×10 mL), dried over anhydrous Na₂SO₄. After filtration, the filtrate was concentrated under reduced pressure. The residue was purified by reverse flash chromatography with the following conditions: column, C18 silica; mobile phase, MeCN in water, 10% to 50% gradient in 10 min; detector, UV 254 nm, hold for 30 min at 28%, after evaporated the solvent, methyl 2-(3-cyanophenyl)-3-(4-methylpiperazin-1-yl)propanoate (400 mg, 52.50%) was obtained as a yellow oil. LCMS: m/z (ESI), [M+H]⁺=288.05.

Step 4: To a stirred mixture of methyl 2-(3-cyanophenyl)-3-(4-methylpiperazin-1-yl)propanoate (400 mg, 1.39 mmol, 1 equiv) in THF (8.0 mL) and LiOH·H₂O (175.22 mg, 4.18 mmol, 3.0 equiv) in water (2.0 mL) was added dropwise at room temperature under nitrogen atmosphere. The resulting mixture was stirred for 4.0 h at 25° C. under nitrogen atmosphere. After reaction, the resulting mixture was concentrated under vacuum. The resulting mixture was diluted with methanol (4.0 mL). The residue was purified by reverse flash chromatography with the following conditions: column, C18 silica; mobile phase, MeCN in water, 0% to 50% gradient in 10 min; detector, UV 254 nm, and hold for 20 min at 9.8% to afford 2-(3-cyanophenyl)-3-(4-methylpiperazin-1-yl)propanoic acid I-39 (240 mg, 63.08%) as a white solid. LCMS: m/z (ESI), [M+H]⁺=274.10.

### Synthesis of Intermediate I-40

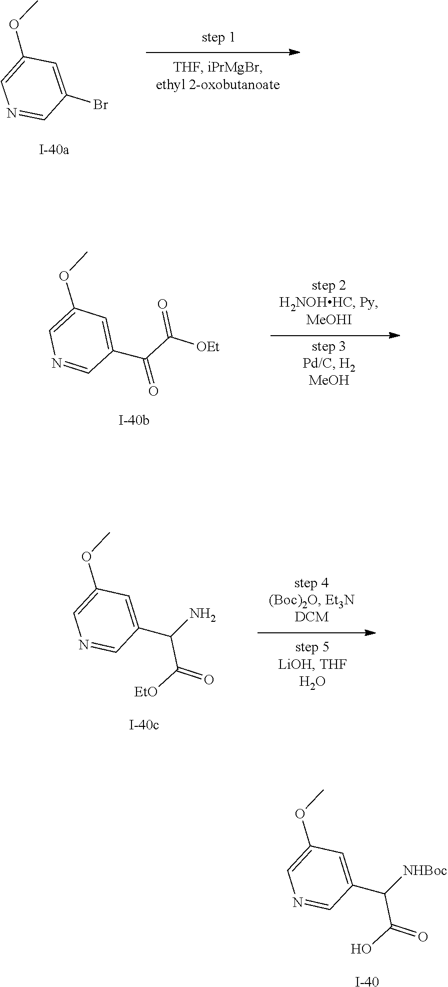

Step 1: Into a 100 mL 3-necked round-bottom flask were added 3-bromo-5-methoxypyridine I-40a (8 g, 42.55 mmol, 1 equiv) and THF (40 mL) at room temperature. Isopropylmagnesium bromide, 1 M solution in THF (17.61 mL, 51.06 mmol, 1.2 equiv) was added to the mixture at −10° C. under nitrogen atmosphere. The mixture was stirred for 30 min at −10° C. under nitrogen atmosphere. Ethyl 2-oxobutanoate (6.64 g, 51.06 mmol, 1.2 equiv) was added to the above mixture at −10° C. The resulting mixture was stirred for 3 h at −8° C. under nitrogen atmosphere. The reaction was quenched by the addition of sat. NH₄Cl (aq.) (50 mL) at 0° C. The resulting mixture was extracted with ethyl acetate (3×30 mL). The combined organic layer was dried over anhydrous Na₂SO₄, filtered and evaporated to afford a crude oil. The residue was purified by silica column chromatography, eluted with PE/EA (1:1, v/v) to afford ethyl 2-(5-methoxypyridin-3-yl)-2-oxoacetate I-40b (4.6 g, 45.48%) as a yellow oil. LCMS: m/z (ESI), [M+H]⁺=210.05. ¹H NMR (CD₃OD, 400 MHZ) δ 1.23 (3H, t), 3.91 (3H, s), 4.15-4.26 (2H, m), 7.61 (1H, dd), 8.23 (1H, d), 8.37 (1H, d).

Step 2: Into a 50 mL round-bottom flask were added ester I-40b (2 g, 9.56 mmol, 1 equiv), hydroxylamine hydrochloride (0.86 g, 12.43 mmol, 1.3 equiv), pyridine (0.83 g, 10.52 mmol, 1.1 equiv) and methanol (10 mL) at 0° C. The resulting mixture was stirred for 6 h at room temperature. The resulting mixture was diluted with CH₂Cl₂ (40 mL). The resulting mixture was washed with 1×40 mL of HCl (0.5M), 1×40 ml of water, and 1×40 mL of saturated NaCl (aq). The organic layer was concentrated under vacuum. The precipitated solids in water phase were collected by filtration and washed with water (3×10 mL). This resulted in ethyl (2E)-2-(N-hydroxyimino)-2-(5-methoxypyridin-3-yl)acetate (1.78 g, 83.04%) as a white solid. LCMS: m/z (ESI), [M+H]⁺=225.15.

Step 3: Into a 50 mL round-bottom flask were added ethyl-2-(N-hydroxyimino)-2-(5-methoxypyridin-3-yl)acetate (1.7 g, 7.58 mmol, 1 equiv), Pd/C (1.86 g, 17.44 mmol, 2.3 equiv) and methanol (100 mL) at room temperature. The resulting mixture was stirred for 6 h at room temperature under hydrogen atmosphere. The resulting mixture was filtered and the filtered cake was washed with methanol (6×30 mL). The filtrate was concentrated under reduced pressure. This resulted in ethyl 2-amino-2-(5-methoxypyridin-3-yl)acetate I-40c (1.6 g, 87.33%) as a yellow oil. LCMS: m/z (ESI), [M+H]⁺=211.10. ¹H NMR (CD₃OD, 400 MHz) δ 1.22 (3H, t), 4.19 (2H, m), 4.69 (1H, d), 4.88 (3H, s), 7.50 (1H, dq), 8.20 (2H, t).

Step 4: Into a 50 mL round-bottom flask were added amine I-40c (1.6 g, 7.61 mmol, 1 equiv), triethylamine (3.85 g, 38.06 mmol, 5 equiv), Boc₂O (3.32 g, 15.22 mmol, 2 equiv) and DCM (5 mL) at room temperature. The resulting mixture was stirred for 2 h at room temperature. The resulting mixture was quenched with water (50 mL), extracted with DCM (2×50 mL). The combined organic layers were washed with saturated brine (1×50 mL), the organic layer was dried over anhydrous Na₂SO₄. After filtration, the filtrate was concentrated under reduced pressure. The residue was purified by prep-TLC with CH₂Cl₂/methanol (25:1, v/v) to afford ethyl 2-[(tert-butoxycarbonyl)amino]-2-(5-methoxypyridin-3-yl)acetate (1.38 g, 49.19%) as a yellow oil. LCMS: m/z (ESI), [M+H]⁺=311.20. ¹H NMR (CD₃OD, 400 MHz) δ 1.19-1.29 (3H, m), 1.47 (9H, s), 3.91 (3H, s), 4.10-4.31 (2H, m), 7.46 (1H, q), 8.10-8.27 (2H, m).

Step 5: A solution of ethyl 2-[(tert-butoxycarbonyl)amino]-2-(5-methoxypyridin-3-yl)acetate (1.38 g, 4.45 mmol, 1 equiv) and LiOH (0.21 g, 8.89 mmol, 2 equiv) in THF (13 mL) and water (13 mL) was stirred for 6 h at 60° C. The mixture/residue was acidified to pH 5 with conc. HCl. The precipitated solids were collected by filtration and washed with methanol (2×10 mL). The filtrate was purified by reverse flash chromatography with the following conditions: column, C18 silica; mobile phase, methanol in water, 0 to 50 gradient in 30 min; detector, UV 254 nm. This resulted in [(tert-butoxycarbonyl)amino](5-hydroxypyridin-3-yl) acetic acid I-40 (500 mg, 41.41%) as a white solid. LCMS: m/z (ESI), [M+H]⁺=283.15.

### Synthesis of Intermediate I-41

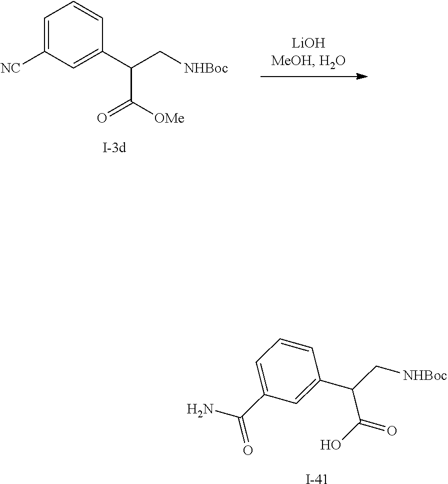

A mixture of methyl 3-[(tert-butoxycarbonyl)amino]-2-(3-cyanophenyl)propanoate I-3d (700 mg, 2.30 mmol, 1 equiv) and LiOH·H₂O (220.34 mg, 9.20 mmol, 4 equiv) in MeOH (8 mL) and H₂O (4 mL) was stirred for 4.5 h at 70° C. under air atmosphere. The residue was purified by reverse flash chromatography with the following conditions: column, C18 silica gel; mobile phase, MeCN in water, 10% to 100% gradient in 50 min; detector, UV 220 nm. This resulted in 3-[(tert-butoxycarbonyl)amino]-2-(3-carbamoylphenyl)propanoic acid I-41 (330 mg, 46.53%) as a yellow solid. LCMS: m/z (ESI), [M−H]⁺=307.05. ¹H NMR (CD₃OD, 400 MHz) δ 1.40 (9H, s), 3.50 (2H, m), 3.73 (1H, t), 7.40 (1H, t), 7.56 (1H, d), 7.73 (1H, d), 7.86 (1H, t).

### Synthesis of Intermediate I-42

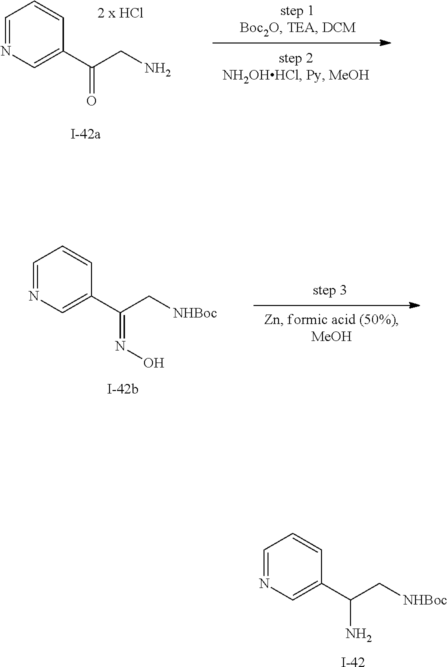

Step 1: To a stirred mixture of 2-amino-1-(pyridin-3-yl) ethanone I-42a (1.0 g, 7.35 mmol, 1 equiv) and Et₃N (2.23 g, 22.03 mmol, 3 equiv) in DCM (20 mL) was added Boc₂O (1.92 g, 8.81 mmol, 1.2 equiv) at room temperature under nitrogen atmosphere. The resulting mixture was stirred for 3 hours at room temperature. After reaction, the resulting mixture was diluted with water (50 mL), extracted with ethyl acetate (3×30 mL). The combined organic layers were dried over anhydrous Na₂SO₄. After filtration, the filtrate was concentrated under reduced pressure. The residue was purified by prep-TLC with PE/EA (1:1, v/v) to afford tert-butyl N-[2-oxo-2-(pyridin-3-yl)ethyl]carbamate (650 mg, 37.46%) as a yellow solid. LCMS: [M+H]⁺=237.05. ¹H NMR (CDCl₃, 400 MHZ) δ 1.52 (9H, s), 4.69 (2H, d), 5.50 (1H, s), 7.48 (1H, ddd), 8.25 (1H, dt), 8.85 (1H, dd), 9.19 (1H, m).

Step 2: To a stirred mixture of tert-butyl N-[2-oxo-2-(pyridin-3-yl)ethyl]carbamate (640 mg, 2.71 mmol, 1 equiv) and hydroxylamine hydrochloride (564.70 mg, 8.13 mmol, 3 equiv) in methanol (10 mL) was added pyridine (642.79 mg, 8.13 mmol, 3 equiv) dropwise at 0° C. under nitrogen atmosphere. The resulting mixture was stirred for overnight room temperature. After reaction, the resulting mixture was quenched by water (20 mL). The resulting mixture was extracted with ethyl acetate (3×20 mL). The combined organic layers were dried over anhydrous Na₂SO₄. After filtration, the filtrate was concentrated under reduced pressure to afford tert-butyl N-[2-(hydroxyimino)-2-(pyridin-3-yl)ethyl]carbamate I-42b (400 mg, 58.77%) as a yellow oil. LCMS: m/z (ESI), [M+H]⁺=252.00.

Step 3: To a stirred mixture of hydroxyimine I-42b (465 mg, 1.85 mmol, 1 equiv) and formic acid (170.34 mg, 3.70 mmol, 2 equiv) in methanol (4.0 mL) was added Zn (362.95 mg, 5.55 mmol, 3 equiv) in portions at room temperature under nitrogen atmosphere. The resulting mixture was stirred for 3 hours at room temperature. Desired product could be detected by LCMS. After reaction. The resulting mixture was concentrated under vacuum. Then diluted with methanol (4.0 mL). The residue was basified to pH 9 with K₂CO₃. The precipitated solids were filtered out and washed with DCM/methanol (10:1, v/v). The filtrate was evaporated and purified with reverse flash chromatography with the following conditions: column, C18 silica; mobile phase, MeCN in water, 10% to 50% gradient in 10 min; detector, UV 220 nm, hold for 20 min at 45%, after evaporated the solvent we afford tert-butyl N-[2-amino-2-(pyridin-3-yl)ethyl]carbamate I-42 (287 mg, 65.36%) as a yellow oil. LCMS: m/z (ESI), [M+H]⁺=238.10. ¹H NMR (DMSO-d₆, 400 MHZ) δ 1.32 (9H, s), 2.05 (2H, s), 3.06 (2H, m), 3.90 (1H, t), 6.86 (1H, t), 7.32 (1H, dd), 7.73 (1H, dt), 8.41 (1H, dd), 8.49 (1H, d).

### Synthesis of Intermediate I-43

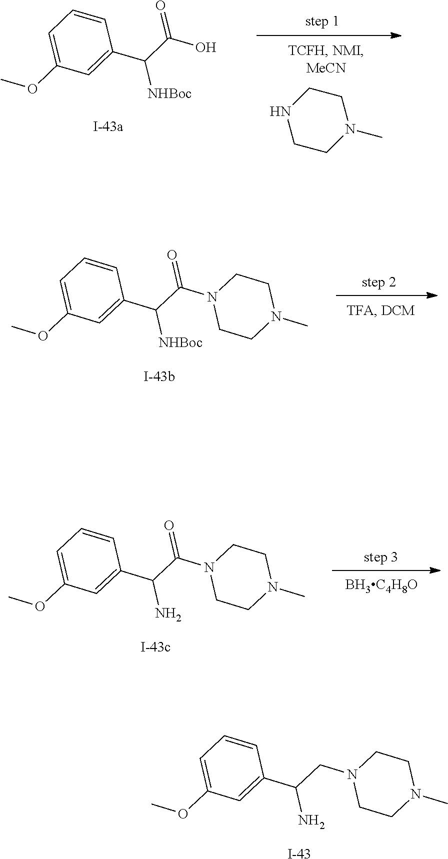

Step 1: Into a 250 mL round-bottom flask were added [(tert-butoxycarbonyl)amino](3-methoxyphenyl) acetic acid I-43a (3 g, 10.66 mmol, 1 equiv) and piperazine, 1-methyl-(1.28 g, 12.80 mmol, 1.2 equiv), TCFH (4.49 g, 16.00 mmol, 1.5 equiv), NMI (4.38 g, 53.32 mmol, 5 equiv) in MeCN (30 mL) at room temperature. The resulting mixture was stirred for 3 h at room temperature under nitrogen atmosphere. The residue was purified by reverse flash chromatography with the following conditions: column, C18 silica; mobile phase, methanol in water, 0% to 70% gradient in 40 min; detector, UV 254 nm to afford tert-butyl N-[1-(3-methoxyphenyl)-2-(4-methylpiperazin-1-yl)-2-oxoethyl]carbamate I-43b (2.5 g, 64.50%) as a yellow oil. LCMS: m/z (ESI), [M+H]⁺=364.20.

Step 2: Into a 50 mL round-bottom flask were added amide I-43b (2.2 g, 6.05 mmol, 1 equiv) and TFA (4 mL, 53.85 mmol, 8.90 equiv) in DCM (4 mL) at room temperature. The resulting mixture was stirred for 15 h at room temperature under nitrogen atmosphere. The resulting mixture was concentrated under reduced pressure. The residue was basified to pH 9 with NH₃—H₂O. The residue was purified by reverse flash chromatography with the following conditions: column, C18 silica; mobile phase, methanol in water (5 mmol/L NH₄HCO₃), 10% to 50% gradient in 10 min; detector, UV 254 nm to afford 2-amino-2-(3-methoxyphenyl)-1-(4-methylpiperazin-1-yl) ethanone I-43c (1.5 g, 94.10%) as a yellow oil. LCMS: m/z (ESI), [M+H−^{t}Bu]⁺=264.05. ¹H NMR (CD₃OD, 400 MHz) δ 2.62 (1H, d), 2.79 (3H, s), 3.16 (3H, d), 3.44 (1H, s), 3.85 (5H, s), 4.03 (1H, s), 5.55 (1H, s), 7.03-7.18 (3H, m), 7.45 (1H, t).

Step 3: Into a 40 mL vial were added amine I-43c (1.5 g, 5.70 mmol, 1 equiv) and BH₃·C₄H₈O (20 mL, 208.98 mmol, 36.69 equiv) at room temperature. The resulting mixture was stirred for 24 h at 70° C. under nitrogen atmosphere. The mixture was allowed to cool down to room temperature. The reaction was quenched by the addition of methanol (20 mL) at 0° C. And The resulting mixture was stirred for 2 h, concentrated under reduced pressure and The residue was purified by reverse flash chromatography with the following conditions: column, C18 silica; mobile phase, methanol in water, 0% to 50% gradient in 40 min; detector, UV 220 nm to afford 1-(3-methoxyphenyl)-2-(4-methylpiperazin-1-yl) ethanamine I-43 (500 mg, 35.20%) as a colorless oil. LCMS: m/z (ESI), [M+H−^{t}Bu]⁺=250.05.

### Synthesis of Intermediate I-44

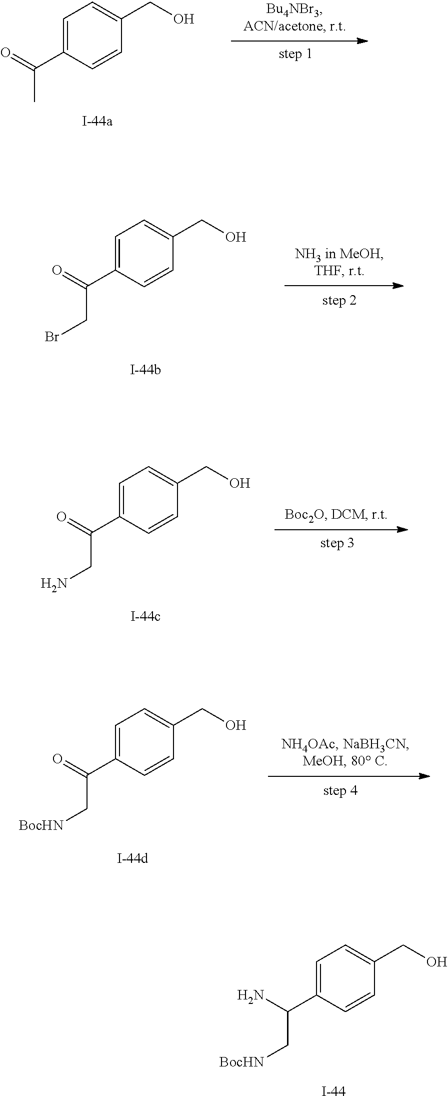

Step 1: A mixture of tribromane-tetrabutylamine (6.42 g, 13.32 mmol, 1 equiv) and 1-[4-(hydroxymethyl)phenyl]ethanone I-44a (2 g, 13.32 mmol, 1 equiv) in ACN (100 mL) and acetone (10 mL) was stirred for 1 h at room temperature under nitrogen atmosphere. The resulting mixture was concentrated under reduced pressure. The resulting mixture was diluted with water (10 mL). The resulting mixture was extracted with MTBE (2×20 mL). The combined organic layers were washed with water (7×20 mL), dried over anhydrous Na₂SO₄. After filtration, the filtrate was concentrated under reduced pressure to afford 2-bromo-1-[4-(hydroxymethyl)phenyl]ethanone I-44b (2 g, 65.56%) as a yellow oil. The resulting mixture was used in the next step directly.

Step 2: A mixture of NH₃ (g) in methanol (6.24 mL, 43.66 mmol, 5 equiv) and bromide I-44b (2 g, 8.73 mmol, 1 equiv) in THF (50 mL) was stirred for 2 h at room temperature under nitrogen atmosphere. The resulting mixture was concentrated under reduced pressure. The residue was purified by reverse flash chromatography with the following conditions: column, C18 silica; mobile phase, MeCN in water (0.1% NH₃·H₂O), 0% to 50% gradient in 60 min; detector, UV 254 nm to afford 2-amino-1-[4-(hydroxymethyl)phenyl]ethanone I-44c (1 g, 69.34%) as a colorless oil. ¹H NMR (400 MHZ, DMSO-d₆) δ 2.56 (2H, d), 4.62 (2H, s), 5.43 (1H, s), 7.53 (2H, d), 8.00 (2H, d), 8.21 (2H, s).

Step 3: A mixture of Boc₂O (1.32 g, 6.05 mmol, 1 equiv), triethylamine (1.84 g, 18.16 mmol, 3 equiv) and amine I-44c (1 g, 6.05 mmol, 1 equiv) in DCM (20 mL) was stirred for 2 h at room temperature under nitrogen atmosphere. The resulting mixture was concentrated under reduced pressure. The residue was purified by prep-TLC water PE/EA (1:2, v/v) to afford tert-butyl N-{2-[4-(hydroxymethyl)phenyl]-2-oxoethyl}carbamate I-44d (500 mg, 31.13%) as a yellow solid. ¹H NMR (400 MHZ, DMSO-d₆) δ 1.40 (9H, s), 4.43 (2H, d), 4.59 (2H, s), 5.38 (1H, s), 7.05 (1H, t), 7.47 (2H, d), 7.89-8.00 (2H, m).

Step 4: A mixture of NaBH₃CN (278.31 mg, 4.43 mmol, 2.5 equiv), NH₄OAc (682.77 mg, 8.86 mmol, 5 equiv) and ketone I-44d (470 mg, 1.77 mmol, 1 equiv) in methanol (10 mL) was stirred for 2 h at 80° C. under nitrogen atmosphere. The residue was purified by reverse flash chromatography with the following conditions: column, C18 silica; mobile phase, methanol in water (10 mmol/L NH₄HCO₃), 0% to 100% gradient in 60 min; detector, UV 254 nm to afford tert-butyl N-{2-amino-2-[4-(hydroxymethyl)phenyl]ethyl}carbamate I-44 (110 mg, 23.31%) as a yellow oil. ¹H NMR (400 MHZ, DMSO-d₆) δ 1.41 (9H, s), 4.43 (2H, d), 4.59 (3H, d), 5.37 (1H, t), 7.04 (1H, t), 7.47 (2H, d), 7.94 (2H, s).

### Synthesis of Intermediate I-45

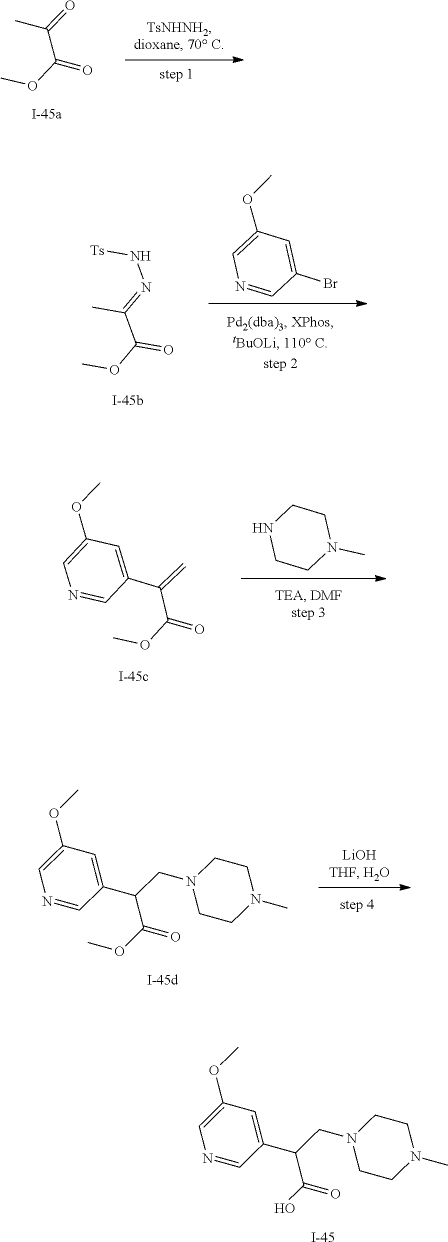

Step 1: A solution of methylpyruvate I-45a (5 g, 48.98 mmol, 1 equiv) and 4-methylbenzenesulfonohydrazide (10.03 g, 53.88 mmol, 1.1 equiv) in 1,4-dioxane (30 mL) was stirred for 2 h at 70° C. under nitrogen atmosphere. The resulting mixture was concentrated under reduced pressure. The residue was purified by trituration with methanol (20 mL), to afford methyl [N′-(4-methylbenzenesulfonyl) hydrazinecarbonyl]formate I-45b (12.8 g, 95.98%) as an off-white solid. LCMS: m/z (ESI), [M+NH₄]⁺=288.05.

Step 2: Into a 40 mL vial were added ester I-45b (5000 mg, 18.36 mmol, 1 equiv), Pd₂(dba)₃ (840.8 mg, 0.92 mmol, 0.05 equiv), XPhos (1750.9 mg, 3.67 mmol, 0.2 equiv), (tert-butoxy) lithium (3528.2 mg, 44.06 mmol, 2.4 equiv) and 3-bromo-5-methoxypyridine (3452.8 mg, 18.36 mmol, 1.0 equiv) in dioxane (100 mL) at room temperature. The resulting mixture was stirred for 20 min at 110° C. under nitrogen atmosphere. The reaction was quenched with sat. NH₄Cl (aq.) at 0° C. The resulting mixture was quenched with water (100 mL), extracted with ethyl acetate (3×100 mL). The combined organic layers were dried over anhydrous Na₂SO₄, filtered and evaporated to afford a crude solid. The residue was purified by prep-TLC with CH₂Cl₂/methanol (45:1, v/v) to afford methyl 2-(5-methoxypyridin-3-yl)prop-2-enoate I-45c (1500 mg, 42.28%) as a yellow oil. LCMS: m/z (ESI), [M+H]⁺=193.95.

Step 3: Into a 50 mL round-bottom flask were added enoate I-45c (1.5 g, 7.76 mmol, 1 equiv), triethylamine (2.36 g, 23.29 mmol, 3 equiv) and 1-methylpiperazine (777.65 mg, 7.76 mmol, 1 equiv) in DMF (3 mL) at room temperature. The resulting mixture was stirred for 1.5 h at room temperature under nitrogen atmosphere. The resulting mixture was concentrated under vacuum. The residue was purified by reverse flash chromatography with the following conditions: column, C18 silica; mobile phase, methanol in water, 0% to 100% gradient in 30 min; detector, UV 254 nm. The resulting mixture was concentrated under vacuum, to afford methyl 2-(5-methoxypyridin-3-yl)-3-(4-methylpiperazin-1-yl)propanoate I-45d (900 mg, 39.51%) as a white oil. LCMS: m/z (ESI), [M+H]⁺=294.10.

Step 4: Into a 50 mL round-bottom flask were added methylpiperazine I-45d (900 mg, 3.07 mmol, 1 equiv), THF (2 mL) and LiOH·H₂O (257.45 mg, 6.14 mmol, 2 equiv) in water (2 mL) at room temperature. The resulting mixture was stirred for 2 h at room temperature. The mixture was acidified to pH 5 with HCl (aq.). The residue was purified by reverse flash chromatography with the following conditions: column, C18 silica; mobile phase, methanol in water, 0% to 100% gradient in 30 min; detector, UV 254 nm. The resulting mixture was concentrated under vacuum, to afford 2-(5-methoxypyridin-3-yl)-3-(4-methylpiperazin-1-yl)propanoic acid I-45 (330 mg, 38.51%) as a white solid. LCMS: m/z (ESI), [M+H]⁺=280.10. ¹H NMR (DMSO-d₆, 400 MHZ) δ 2.70 (3H, s), 3.05 (3H, dd), 3.36 (7H, s), 3.84 (3H, s), 3.94 (1H, t), 7.34 (1H, dd), 8.16 (1H, d), 8.22 (1H, d).

### Synthesis of Intermediate I-46

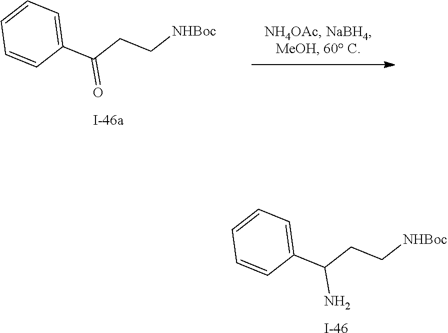

Into a 20 mL vial were added tert-butyl N-(3-oxo-3-phenylpropyl) carbamate I-46a (400 mg, 1.60 mmol, 1 equiv), sodium cyanoboranuide (403.30 mg, 6.42 mmol, 4 equiv) and NH₄OAc (1.24 g, 16.04 mmol, 10 equiv) in methanol (20.00 mL) at room temperature. The resulting mixture was stirred for overnight at 60° C. under nitrogen atmosphere. The mixture basified to pH 9 with NaOH (2 mol/L). The resulting mixture was extracted with ethyl acetate (3×100 mL). The combined organic layers were dried over anhydrous Na₂SO₄, filtered and concentrated under reduced pressure. The residue was purified by prep-TLC with CH₂Cl₂/methanol (15:1, v/v) to afford tert-butyl N-(3-amino-3-phenylpropyl) carbamate I-46 (250 mg, 62.24%) as a yellow oil. LCMS: m/z (ESI), [M+H]⁺=251.15. ¹H NMR (DMSO-d₆, 400 MHz) δ 1.37 (9H, s), 1.66-1.80 (2H, m), 2.89 (2H, p), 3.89 (1H, t), 7.25 (1H, ddt), 7.29-7.41 (4H, m).

### Synthesis of Intermediate I-47

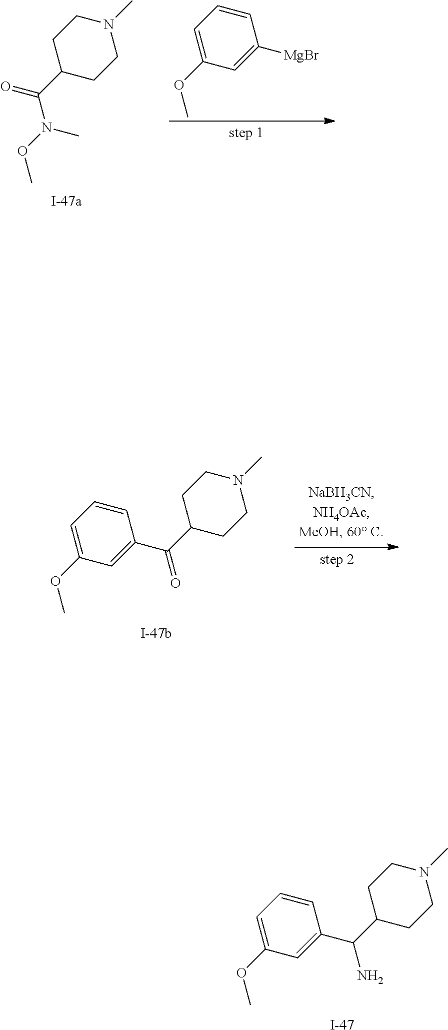

Step 1: To a stirred mixture of N-methoxy-N, 1-dimethylpiperidine-4-carboxamide I-47a (1.0 g, 5.37 mmol, 1 equiv) in THF (10 mL) was added bromo-(3-methoxyphenyl) magnesium (1.16 g, 5.48 mmol, 1.02 equiv) dropwise at 0° C. under air atmosphere. The resulting mixture was stirred for overnight at room temperature under air atmosphere. After reaction, the reaction was quenched with sat. NH₄Cl (aq.) (40 mL) at 0° C. The resulting mixture was extracted with ethyl acetate (3×40 mL). The combined organic layers were washed with water (3×40 mL), dried over anhydrous Na₂SO₄. After filtration, the filtrate was concentrated under reduced pressure. The residue was purified by silica column chromatography, eluted with CH₂Cl₂/methanol (50:1, v/v) to afford 4-(3-methoxybenzoyl)-1-methylpiperidine I-47b (900 mg, 71.85%) as a white solid. LCMS: m/z (ESI), [M+H]⁺=234.10. ¹H NMR (CDCl₃, 400 MHz) δ 1.90 (4H, m), 2.14 (2H, m), 2.34 (3H, s), 2.96 (2H, dd), 3.22 (1H, tt), 3.87 (3H, s), 7.12 (1H, ddd), 7.39 (1H, t), 7.50 (2H, m).

Step 2: To a stirred mixture of methylpiperidine I-47b (880 mg, 3.77 mmol, 1 equiv) in methanol (10 mL) was added NaBH₃CN (948.08 mg, 15.09 mmol, 4 equiv) and CH₃COONH₄ (3488.89 mg, 45.26 mmol, 12 equiv) in portions at 0° C. under air atmosphere. The resulting mixture was stirred for overnight at 70° C. under air atmosphere. After cooling to room temperature, the reaction was quenched by the addition of sat. NH₄Cl (aq.) (30 mL) at 0° C. The resulting mixture was extracted with CH₂Cl₂ (3×40 mL). The combined organic layers were washed with water (3×10 mL), dried over anhydrous Na₂SO₄. After filtration, the filtrate was concentrated under reduced pressure. The residue was purified by reverse flash chromatography with the following conditions: column, C18 silica; mobile phase, MeCN in water, 10% to 50% gradient in 10 min; detector, UV 254 nm hold for 25 min at 40% to afford 1-(3-methoxyphenyl)-1-(1-methylpiperidin-4-yl) methanamine I-47 (340 mg, 38.47%) as a yellow solid. LCMS: m/z (ESI), [M+H]⁺=235.15.

### Synthesis of Intermediate I-48

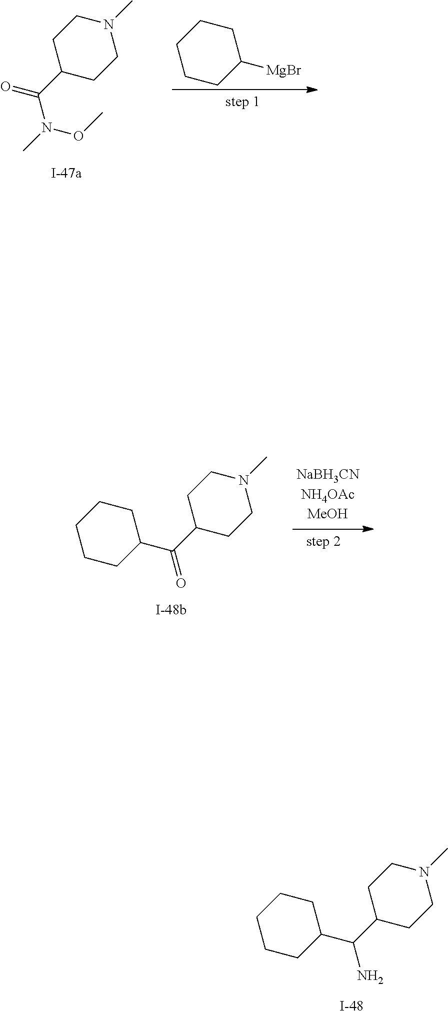

Step 1: Into a 40 mL vial were added N-methoxy-N, 1-dimethylpiperidine-4-carboxamide I-47a (2 g, 10.74 mmol, 1 equiv) and bromo (cyclohexyl) magnesium (21.48 mL, 21.48 mmol, 2 equiv) in THF (10.00 mL) at room temperature. The resulting mixture was stirred for overnight at room temperature under nitrogen atmosphere. The reaction was quenched with water at room temperature. The resulting mixture was extracted with ethyl acetate (3×20 mL). The combined organic layers were concentrated under reduced pressure. The residue was purified by silica column chromatography, eluted with CH₂Cl₂/methanol (20:1, v/v) to afford 4-cyclohexanecarbonyl-1-methylpiperidine I-48b (270 mg, 12.01%) as a yellow oil. LCMS: m/z (ESI), [M+H]⁺=210.20. ¹H NMR (DMSO-d₆, 400 MHz) δ 1.08-1.32 (5H, m), 1.35-1.48 (2H, m), 1.56-1.78 (7H, m), 1.86 (2H, td), 2.14 (3H, s), 2.46 (1H, ddt), 2.56 (1H, td), 2.75 (2H, dt).

Step 2: Into a 20 mL vial were added methylpiperidine I-48b (260 mg, 1.24 mmol, 1 equiv), NH₄OAc (1.34 g, 17.39 mmol, 14 equiv) and sodium cyanoboranuide (312.21 mg, 4.97 mmol, 4 equiv) in methanol (5 mL) at room temperature. The resulting mixture was stirred for overnight at 60° C. under nitrogen atmosphere. The residue was purified by reverse flash chromatography with the following conditions: column, C18 silica; mobile phase, water in MEOH, 0% to 100% gradient in 30 min; detector, UV 254 nm. The resulting mixture was concentrated under vacuum, to afford 1-cyclohexyl-1-(1-methylpiperidin-4-yl) methanamine I-48 (180 mg, 68.89%) as a brown oil. LCMS: m/z (ESI), [M+H]⁺=211.20. ¹H NMR (DMSO-d₆, 400 MHz) δ 0.96 (1H, qd), 1.04-1.12 (1H, m), 1.17 (4H, ddt), 1.22-1.34 (3H, m), 1.44 (2H, ddd), 1.54-1.79 (7H, m), 2.08 (1H, d), 2.11 (3H, s), 2.70-2.81 (2H, m).

### Synthesis of Intermediate I-49

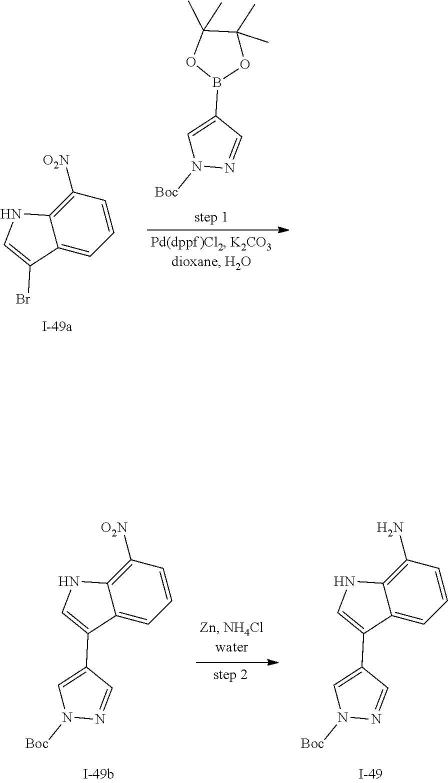

Step 1: Into a 250 mL round-bottom flask were added 3-bromo-7-nitro-1H-indole I-49a (20 g, 82.97 mmol, 1 equiv), tert-butyl 4-(4,4,5,5-tetramethyl-1,3,2-dioxaborolan-2-yl) pyrazole-1-carboxylate (26.85 g, 91.27 mmol, 1.1 equiv), K₂CO₃ (34.40 g, 248.92 mmol, 3 equiv), Pd(dppf)Cl₂ (3.04 g, 4.15 mmol, 0.05 equiv), 1,4-dioxane (80 mL, 0.023 mmol) and water (20 mL, 27.76 mmol) at room temperature. The resulting mixture was stirred for 2 h at 80° C. under nitrogen atmosphere. The residue was purified by trituration with water (30 mL). This resulted a crude solid (26 g), the crude solid was triturated with methanol (50 mL), and this resulted tert-butyl 4-(7-nitro-1H-indol-3-yl) pyrazole-1-carboxylate I-49b (17 g, 39.31%) as a brown yellow solid. LCMS: m/z (ESI), [M-Boc]⁺=229.05.

Step 2: Into a 100 mL round-bottom flask were added nitro-indole I-49b (2.5 g, 7.61 mmol, 1 equiv) and Zn (3.98 g, 60.91 mmol, 8 equiv) at 0° C. A solution of NH₄Cl (1.79 g, 33.51 mmol, 5 equiv) in water (10 mL) was added to the above mixture at 0° C. The resulting mixture was stirred for 1 h at 0° C. under nitrogen atmosphere. The resulting mixture was filtered and the filtered cake was washed with ethyl acetate (2×20 mL). The filtrate was diluted with water (50 mL) and the residue was extracted with EA (2×50 mL), the combined organic layer was dried over anhydrous Na₂SO₄, filtered and evaporated to afford a crude solid. The residue was purified by prep-TLC with CH₂Cl₂/methanol (25:1, v/v) to afford tert-butyl 4-(7-amino-1H-indol-3-yl) pyrazole-1-carboxylate I-49 (1 g, 44.02%) as a brown solid. LCMS: m/z (ESI), [M−^{t}Bu]⁺=243.10.

### Synthesis of Intermediate I-50

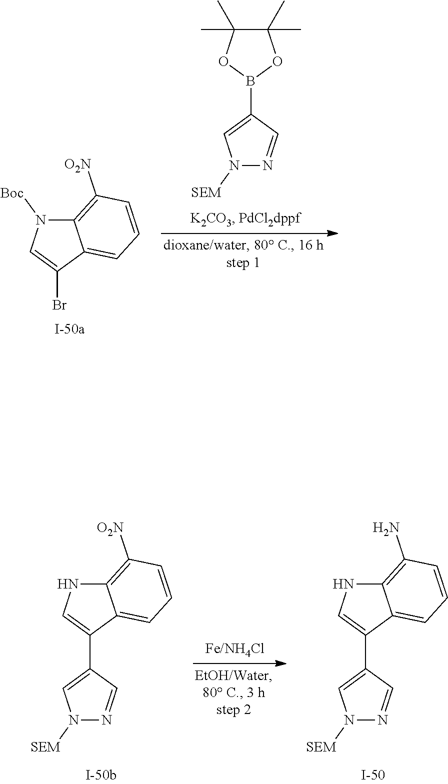

Step 1: tert-butyl 3-bromo-7-nitro-indole-1-carboxylate I-50a (219 mg, 641.93 μmol), 4-(4,4,5,5-tetramethyl-1,3,2-dioxaborolan-2-yl)-1-((2-(trimethylsilyl) ethoxy)methyl)-1H-pyrazole (249.81 mg, 770.32 μmol), potassium carbonate (266.16 mg, 1.93 mmol) and Pd(dppf)Cl₂ (52.42 mg, 64.19 μmol) in dioxane (5.00 mL) and water (1.00 mL) were stirred under nitrogen at 90° C. for 16 h. The solvent was removed under reduced pressure. The residue was purified by silica column chromatography, elution gradient from 0 to 10% methanol in DCM. Pure fractions were evaporated to dryness to afford trimethyl-[2-[[4-(7-nitro-1H-indol-3-yl) pyrazol-1-yl]methoxy]ethyl]silane I-50b (200 mg, 87% yield) as brown solid. LCMS: m/z (ESI), [M+H]⁺=359.3.

Step 2: To a mixture of nitro-indole I-50b (200 mg, 557.93 μmol) and ammonia hydrochloride (298.45 mg, 5.58 mmol) in ethanol (10 mL) and water (10 mL) was added Iron powder (155.79 mg, 2.79 mmol). Then the reaction mixture was stirred and heated at 80° C. for 3 h. The reaction mixture was filtered through celite and washed with ethanol (3×10 mL), and the filtrate was concentrated under reduced pressure. The residue was dissolved 50 mL of ethyl acetate, filtered and the filtration was concentrated under reduced pressure to afford 3-[1-(2-trimethylsilylethoxymethyl) pyrazol-4-yl]-1H-indol-7-amine I-50 (160 mg, 87% yield) as brown solid. LCMS: m/z (ESI), [M+H]⁺=329.3.

### Synthesis of Intermediate I-51

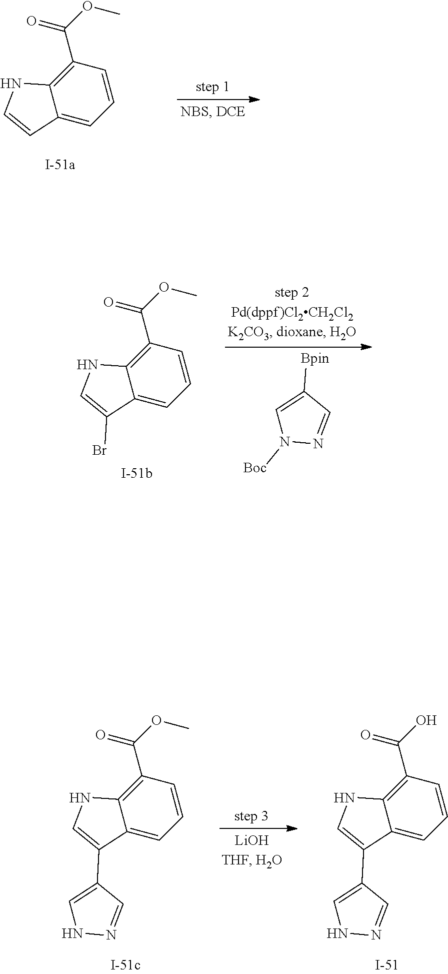

Step 1: Into a 50 mL 3-necked round-bottom flask were added methyl 1H-indole-7-carboxylate I-51a (5 g, 28.54 mmol, 1 equiv) and DCE (50 mL) at −10° C. To the stirred mixture added NBS (6.10 g, 34.25 mmol, 1.2 equiv) in portions at −10° C. under nitrogen atmosphere. The resulting mixture was quenched with water (80 mL), extracted with CH₂Cl₂ (3×50 mL). The combined organic layer was dried over anhydrous Na₂SO₄. After filtration, the filtrate was concentrated under reduced pressure. The residue was purified by silica column chromatography, eluted with PE/EA (5:1, v/v) to afford methyl 3-bromo-1H-indole-7-carboxylate I-51b (5.1 g, 68.71%) as a white solid. LCMS: m/z (ESI), [M+H]⁺=253.95. ¹H NMR (400 MHZ, DMSO-d₆) δ 3.95 (3H, s), 7.27 (1H, t), 7.60 (1H, d), 7.74 (1H, dt), 7.88 (1H, dd), 11.51 (1H, s).

Step 2: A solution of bromide I-51b (2 g, 7.87 mmol, 1 equiv), Pd(dppf)Cl₂·CH₂Cl₂ (0.64 g, 0.79 mmol, 0.1 equiv), K₂CO₃ (3.26 g, 23.61 mmol, 3 equiv), and 4-(4,4,5,5-tetramethyl-1,3,2-dioxaborolan-2-yl)-1H-pyrazole (1.53 g, 7.87 mmol, 1 equiv) in 1,4-dioxane (4 mL), water (1 mL) was stirred for 12 h at 80° C. under nitrogen atmosphere. The resulting mixture was diluted with water (30 mL), extracted with ethyl acetate (3×30 mL), dried over anhydrous Na₂SO₄. After filtration, the filtrate was concentrated under reduced pressure. The residue was purified by silica column chromatography, eluted with CH₂Cl₂/methanol (5:1, v/v) to afford methyl 3-[1-(tert-butoxycarbonyl) pyrazol-4-yl]-1H-indole-7-carboxylate I-51c (810 mg, 19.41%) as a yellow oil. LCMS: m/z (ESI), [M+H]⁺=242.00.

Step 3: Into a 50 mL round-bottom flask were added ester I-51c (390 mg, 1.14 mmol, 1 equiv), LiOH (136.81 mg, 5.71 mmol, 5 equiv), THF (2.5 mL) and water (2.5 mL) at room temperature. The resulting mixture was stirred for 8 h at room temperature. The mixture was acidified to pH 5 with HCl (1M). The residue was purified by reverse flash chromatography with the following conditions: column, C18 silica; mobile phase, methanol in water, 0% to 20% gradient in 30 min; detector, UV 254 nm. This resulted in 3-(1H-pyrazol-4-yl)-1H-indole-7-carboxylic acid I-51 (60 mg, 13.98%) as a yellow solid. LCMS: m/z (ESI), [M+H]⁺=242.10.

### Synthesis of Intermediate I-52

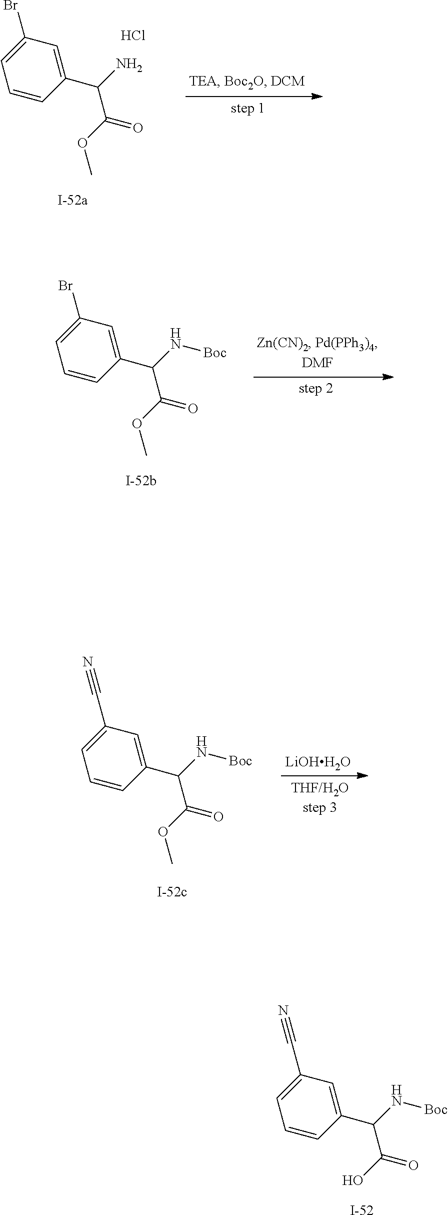

Step 1: A mixture of methyl 2-amino-2-(3-bromophenyl)acetate I-52a (2.0 g, 8.19 mmol, 1 equiv) in DCM (30 mL) was added (Boc)₂O (3576.56 mg, 16.39 mmol, 2 equiv), TEA (2487.46 mg, 24.58 mmol, 3 equiv). The resulting mixture was stirred for 4 h at room temperature under air atmosphere. After reaction, the resulting mixture was concentrated under vacuum and resolved with DMF (4 mL). The residue was purified by reverse flash chromatography with the following conditions: column, C18 silica; mobile phase, MeOH in water, 10% to 100% gradient in 10 min; detector, UV 254 nm, hold for 30 min at 90% to afford methyl 2-(3-bromophenyl)-2-[(tert-butoxycarbonyl)amino]acetate I-52b (1.65 g, 58.50% yield) as a white solid. LCMS: m/z (ESI), [M-Boc]⁺=243.95.

Step 2: A mixture of bromide I-52b (1.2 g, 3.49 mmol, 1 equiv) in DMF (4.0 mL) was added Zn(CN)₂ (417.55 mg, 3.56 mmol, 1.02 equiv), Pd(PPh₃)₄ (402.87 mg, 0.35 mmol, 0.1 equiv). The resulting mixture was stirred for 2 h at 70° C. under nitrogen atmosphere. After cooling to room temperature, the resulting mixture was diluted with water (40 mL). The resulting mixture was extracted with EtOAc (3×40 mL). The combined organic layers were washed with water (3×10 mL), dried over anhydrous Na₂SO₄. After filtration, the filtrate was concentrated under reduced pressure. The residue was purified by prep-TLC with PE/EA (3:1, v/v) to afford methyl 2-[(tert-butoxycarbonyl)amino]-2-(3-cyanophenyl)acetate I-52c (990 mg, 97.81% yield) as a white solid. LCMS: m/z (ESI), [M-Boc+H]⁺=191.00. ¹H NMR (DMSO-d₆, 400 MHz) δ 1.40 (9H, s), 3.72 (3H, s) 5.38 (1H, d), 7.58 (1H, t), 7.75 (1H, m), 7.81 (1H, d), 7.88 (1H, d), 7.94 (1H, d).

Step 3: A mixture of ester I-52c (990 mg, 3.41 mmol, 1 equiv) in THF (4.0 mL) was added LiOH·H₂O (572.34 mg, 13.64 mmol, 4 equiv) in water (1.0 mL). The resulting mixture was stirred for 6 h at room temperature under nitrogen atmosphere. After reaction, the resulting mixture was concentrated under vacuum and diluted with DMF (2 mL). The residue was purified by reverse flash chromatography with the following conditions: column, C18 silica; mobile phase, MeCN in water, 10% to 50% gradient in 10 min; detector, UV 254 nm, hold for 25 min at 25%, after evaporated the water to afford [(tert-butoxycarbonyl)amino](3-cyanophenyl) acetic acid I-52 (540 mg, 57.31% yield) as a white solid. LCMS: m/z (ESI), [M+H]⁺=277.00.

### Synthesis of Intermediate I-53

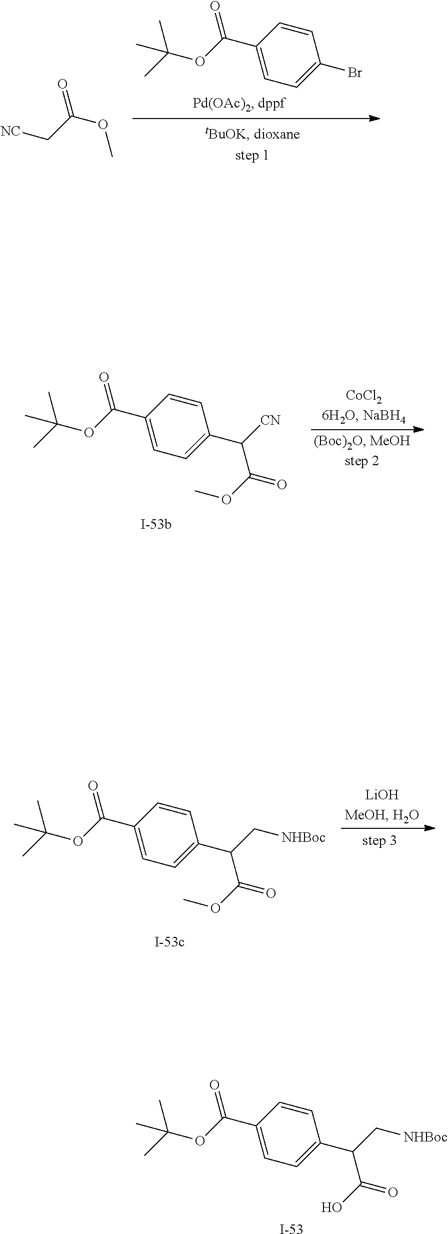

Step 1: To a stirred solution of tert-butyl 4-bromobenzoate (20.11 g, 78.21 mmol, 1.55 equiv) and methyl cyanoacetate (5 g, 50.46 mmol, 1.00 equiv) in dioxane (40 mL) were added t-BuOK (14.16 g, 126.19 mmol, 2.50 equiv) and Pd(OAc)₂ (1.13 g, 5.03 mmol, 0.10 equiv) dppf (5.57 g, 10.08 mmol, 0.20 equiv) at room temperature under air atmosphere. The resulting mixture was stirred for 3 h at 70° C. under nitrogen atmosphere. The reaction was quenched by the addition of water (100 mL) at room temperature. The resulting mixture was extracted with EtOAc (3×50 mL). The combined organic layers were dried over anhydrous Na₂SO₄. After filtration, the filtrate was concentrated under reduced pressure. The residue was purified by silica column chromatography, eluted with PE/EA (1:1, v/v) to afford tert-butyl 4-(1-cyano-2-methoxy-2-oxoethyl)benzoate I-53b (3.77 g, 27.14% yield) as a yellow oil. ¹H NMR: (DMSO-d₆, 400 MHz) δ 1.55 (9H, s), 3.72 (3H, d), 5.84 (1H, s), 7.54-7.61 (2H, m), 7.94-8.02 (2H, m).

Step 2: To a stirred solution of nitrile I-53b (3.77 g, 13.69 mmol, 1 equiv) and CoCl₂·6H₂O (9.77 g, 41.08 mmol, 3 equiv) and (Boc) 20 (8.97 g, 41.08 mmol, 3 equiv) in MeOH (50 mL) were added NaBH₄ (4.14 g, 109.55 mmol, 8 equiv) in portions at 0° C. The resulting mixture was stirred for additional overnight at room temperature. The reaction was quenched by the addition of ice/water (50 mL) at room temperature. The precipitated solids were filtered out and washed with DCM (3×20 mL). The resulting mixture was extracted with CH₂Cl₂ (3×50 mL). The combined organic layers were dried over anhydrous Na₂SO₄. After filtration, the filtrate was concentrated under reduced pressure. The residue was purified by silica column chromatography, eluted with PE/EA (1:1, v/v) to afford tert-butyl 4-{3-[(tert-butoxycarbonyl)amino]-1-methoxy-1-oxopropan-2-yl}benzoate I-53c (2947 mg, 56.71% yield) as a yellow oil. LCMS: m/z (ESI), [M+H]⁺=380.45. ¹H NMR: (CD₃OD, 400 MHz) δ 1.41 (9H, s), 1.61 (9H, s), 3.43 (1H, dd), 3.63 (1H, dd), 3.70 (3H, s), 3.98 (1H, t), 7.40 (2H, d), 7.89-7.97 (2H, m).

Step 3: To a stirred solution of ester I-53c (2937 mg, 7.74 mmol, 1 equiv) in MeOH (20 mL) and H₂O (10 mL) was added LiOH (741.50 mg, 30.96 mmol, 4 equiv) at room temperature under air atmosphere. The resulting mixture was stirred for 3 h at room temperature under air atmosphere. The resulting mixture was concentrated under reduced pressure. The residue was purified by reverse flash chromatography with the following conditions: column, C18 silica; mobile phase, MeCN in water, 90% to 100% gradient in 10 min; detector, UV 254 nm. This resulted in 3-[(tert-butoxycarbonyl)amino]-2-[4-(tert-butoxycarbonyl)phenyl]propanoic acid I-53 (2.5 g, 88.39% yield) as a white solid. LCMS: m/z (ESI), [M+H]⁺=366.20. ¹H NMR: (CD₃OD, 400 MHZ) δ 1.41 (9H, d), 1.60 (8H, s), 2.88 (1H, s), 3.01 (1H, s), 3.44 (1H, dd), 3.53 (1H, dd), 3.73 (1H, q), 7.46 (2H, d), 7.88 (2H, d).

### Synthesis of Intermediate I-54

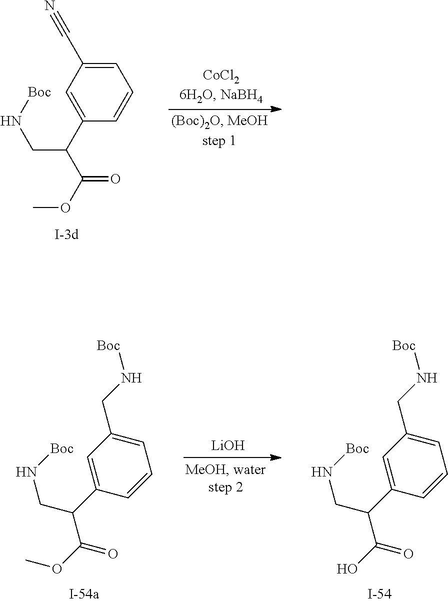

Step 1: To a solution of methyl 3-[(tert-butoxycarbonyl)amino]-2-(3-cyanophenyl)propanoate I-3d (200 mg, 0.66 mmol, 1 equiv) and CoCl₂·6H₂O (469.05 mg, 1.97 mmol, 3 equiv) in MeOH (10 mL) were added Boc₂O (430.26 mg, 1.97 mmol, 3 equiv) at 0° C. under nitrogen atmosphere. The mixture was added NaBH₄ (124.30 mg, 3.29 mmol, 5 equiv) at 0° C. The reaction was stirred for 2 h at 0° C. The resulting mixture was filtered, and the filter cake was washed with MeOH (3×20 mL). The filtrate was concentrated under reduced pressure. The residue was purified by prep-TLC with PE:EA (2:1, v/v) to afford methyl 3-[(tert-butoxycarbonyl)amino]-2-(3-{[(tert-butoxycarbonyl)amino]methyl}phenyl)-propanoate I-54a (180 mg, 47.94% yield) as a white solid. LCMS: m/z (ESI), [M+H]⁺=409.10.

Step 2: To a solution of ester I-54a (180 mg, 0.44 mmol, 1 equiv) in THF (10 mL) and H₂O (5 mL) was added LiOH·H₂O (36.98 mg, 0.88 mmol, 2 equiv) at room temperature under nitrogen atmosphere. The mixture was stirred for 2 h at room temperature. The resulting mixture was concentrated under reduced pressure. The residue was purified by reverse flash chromatography with the following conditions: column, C18 silica; mobile phase, ACN in water, 10% to 50% gradient in 10 min; detector, UV 220 nm. This resulted in 3-[(tert-butoxycarbonyl)amino]-2-(3-{[(tert-butoxycarbonyl)amino]methyl}phenyl)-propanoic acid I-54 (159 mg, 91.27% yield) as a white solid. LCMS: m/z (ESI), [M+H]⁺=395.10.

### Synthesis of Intermediate I-55

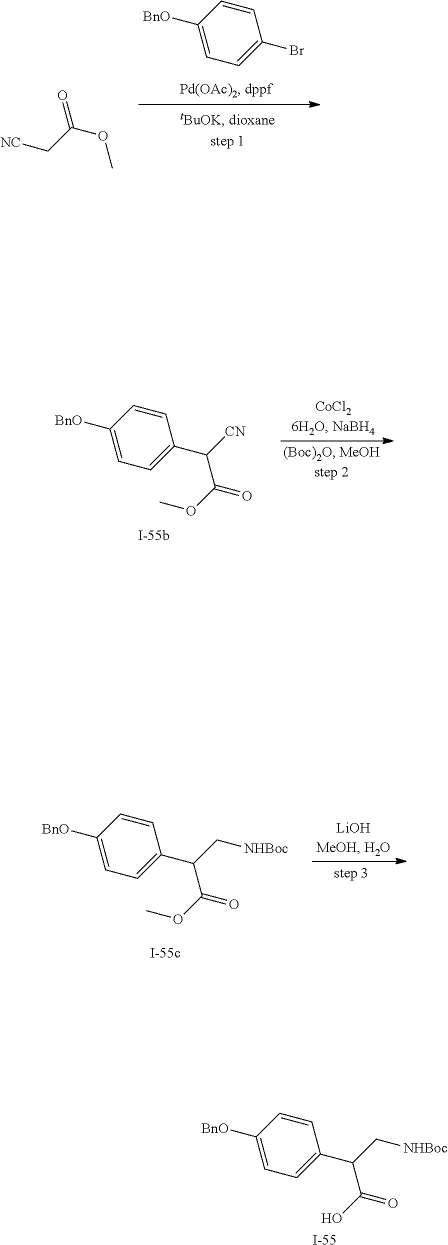

Step 1: To a stirred mixture of 1-(benzyloxy)-4-bromobenzene (8232.15 mg, 31.29 mmol, 1.55 equiv), t-BuOK (5662.23 mg, 50.46 mmol, 2.5 equiv) and Pd(OAc)₂ (453.15 mg, 2.02 mmol, 0.1 equiv) in dioxane (50.00 mL, 590.18 mmol) were added dppf (2229.81 mg, 4.04 mmol, 0.2 equiv) and methyl cyanoacetate (2 g, 20.18 mmol, 1 equiv) dropwise at room temperature under nitrogen atmosphere. The resulting mixture was stirred for 3 h at 70° C. under nitrogen atmosphere. The resulting mixture was concentrated under reduced pressure. The residue was purified by silica column chromatography, eluted with PE/EA (4:1, v/v) to afford methyl 2-[4-(benzyloxy)phenyl]-2-cyanoacetate I-55b (1.955 g, 34.43% yield) as a yellow solid. 1H-NMR: (DMSO-d₆, 400 MHZ) δ 3.72 (3H, s), 5.13 (2H, s), 5.59 (1H, s), 7.09 (2H, m), 7.40 (7H, m).

Step 2: To a stirred mixture of nitrile I-55b (500 mg, 1.78 mmol, 1 equiv) and CoCl₂·6H₂O (1268.63 mg, 5.33 mmol, 3 equiv) in MeOH (2 mL) were added Boc₂O (1163.74 mg, 5.33 mmol, 3 equiv) and NaBH₄ (168.10 mg, 4.44 mmol, 2.5 equiv) in portions at 0° C. under air atmosphere. The resulting mixture was stirred for 1 h at room temperature under air atmosphere. The reaction was quenched with Water at room temperature. The resulting mixture was filtered and the filter cake was washed with MeOH (2×4 mL). The filtrate was concentrated under reduced pressure. The residue was purified by silica column chromatography, eluted with PE/EA (3:1, v/v) to afford methyl 2-[4-(benzyloxy)phenyl]-3-[(tert-butoxycarbonyl)amino]propanoate I-55c (371 mg, 54.15% yield) as a white solid. LCMS: m/z (ESI), [M−^{t}Bu]⁺=330.15.

Step 3: A mixture of ester I-55c (371 mg, 0.96 mmol, 1 equiv) and LiOH·H₂O (121.16 mg, 2.89 mmol, 3 equiv) in THF (4 mL) and H₂O (1 mL) was stirred for 2 h at room temperature under air atmosphere. The resulting mixture was concentrated under reduced pressure. The residue was purified by reverse flash chromatography with the following conditions: column, C18 silica; mobile phase, MeCN in Water, 10% to 50% gradient in 50 min; detector, UV 254 nm. This resulted in 2-[4-(benzyloxy)phenyl]-3-[(tert-butoxycarbonyl)amino]propanoic acid I-55 (150 mg, 41.96% yield) as a yellow solid. LCMS: m/z (ESI), [M−H]=370.10. ¹H NMR: (DMSO-d₆, 400 MHZ) δ 1.34 (9H, s), 3.10-3.24 (3H, m), 5.05 (2H, s), 6.50 (1H, d), 6.82-6.92 (2H, m), 7.10-7.17 (2H, m), 7.28-7.35 (1H, m), 7.35-7.41 (2H, m), 7.41-7.46 (2H, m).

### Synthesis of Intermediate I-56

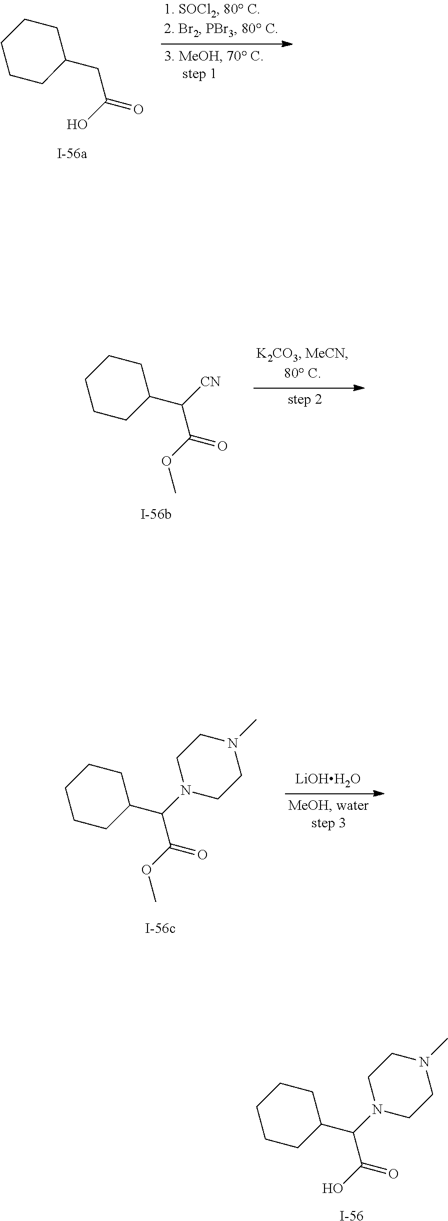

Step 1: Into a 50 mL round-bottom flask were added cyclohexaneacetic acid I-56a (2500 mg, 17.58 mmol, 1.0 equiv) and SOCl₂ (1.5 mL, 20.68 mmol) at room temperature. The resulting mixture was stirred for 1 h at 80° C. under nitrogen atmosphere. The reaction mixture was cooled down to room temperature. To the above mixture was added phosphorus tribromide (4.26 mL, 44.83 mmol, 2.55 equiv), bromine (3512.00 mg, 21.98 mmol, 1.25 equiv) at room temperature. The resulting mixture was stirred for additional 2 h at 80° C. The mixture was allowed to cool down to room temperature. Then MeOH (2.5 mL) was added to the above solution. The resulting mixture was stirred for 2 h at 70° C. under nitrogen atmosphere. The mixture was allowed to cool down to room temperature. The reaction was quenched with sat. NaS₂O₃ (aq) (50 mL) at room temperature. The resulting mixture was extracted with DCM (2×50 mL). The combined organic layer was dried over anhydrous Na₂SO₄, filtered and evaporated to afford a crude solid. The crude solid was purified by silica column chromatography, eluted with PE/EA (5:1, v/v) to afford methyl 2-bromo-2-cyclohexylacetate I-56b (2000 mg, 40.25% yield) as an off-white oil. ¹H NMR: (CDCl₃, 400 MHz) δ 0.90-1.21 (4H, m), 1.27 (2H, qt), 1.60-1.81 (5H, m), 1.88 (1H, tdt), 2.06 (1H, dtd), 3.77 (3H, s), 4.02 (1H, d).

Step 2: Into a 40 mL vial were added bromide I-56b (2000 mg, 8.52 mmol, 1 equiv), 1-methyl-piperazine (1022.44 mg, 10.21 mmol, 1.2 equiv), K₂CO₃ (3526.84 mg, 25.52 mmol, 3.0 equiv) in ACN (20.0 mL) at room temperature. The resulting mixture was stirred for 12 h at 80° C.° C. under nitrogen atmosphere. The mixture was allowed to cool down to room temperature. The resulting mixture was concentrated under reduced pressure. The residue was purified by silica column chromatography, eluted with CH₂Cl₂/MeOH (20:1, v/v) to afford methyl 2-cyclohexyl-2-(4-methylpiperazin-1-yl)acetate I-56c (950 mg, 24.23% yield) as an off-white solid. LCMS: m/z (ESI), [M+H]⁺=255.15. ¹H NMR: (CDCl₃, 400 MHz) δ 0.83-0.99 (3H, m), 1.15-1.32 (4H, m), 1.53 (1H, d), 1.78 (4H, dtd), 1.95 (1H, d), 2.30 (3H, s), 2.53 (6H, dd), 2.64 (2H, dt), 2.89 (1H, d), 3.68 (3H, s).

Step 3: A solution of methylpiperazine I-56c (590 mg, 2.32 mmol, 1 equiv) and LiOH·H₂O (194.66 mg, 4.64 mmol, 2 equiv) in MeOH (3.0 mL) and H₂O (3.0 mL) was stirred for 2 h at 70° C. under nitrogen atmosphere. The mixture was allowed to cool down to room temperature. The mixture was acidified to pH 5 with 1M HCl (aq). The resulting mixture was concentrated under vacuum. The residue was purified by reverse flash chromatography with the following conditions: column, C18 silica; mobile phase, MeOH in water, 10% to 50% gradient in 30 min; detector, UV 220 nm. This resulted in cyclohexyl (4-methylpiperazin-1-yl) acetic acid I-56 (240 mg, 35.8% yield) as an off-white solid. LCMS: m/z (ESI), [M+H]⁺=241.15.

### Synthesis of Intermediate I-57

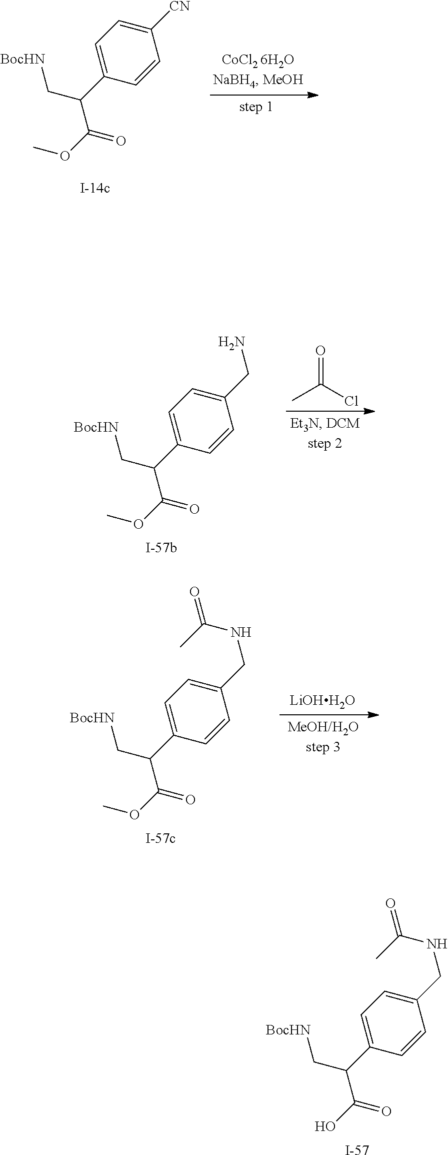

Step 1: To a stirred mixture of methyl 3-[(tert-butoxycarbonyl)amino]-2-(4-cyanophenyl)propanoate I-14c (740 mg, 2.43 mmol, 1 equiv) and CoCl₂·6H₂O (1156.98 mg, 4.86 mmol, 2 equiv) in MeOH (10 mL) was added NaBH₄ (919.81 mg, 24.31 mmol, 10 equiv) in portions at 0° C. under air atmosphere. The resulting mixture was stirred for 5 h at room temperature under air atmosphere. After reaction, the reaction was quenched with water (30 mL) at 0° C. The resulting mixture was filtered and the filter cake was washed with MeOH (3×10 mL). The filtrate was extracted with EtOAc (3×20 mL). The combined organic layers were dried over anhydrous Na₂SO₄. After filtration, the filtrate was concentrated under reduced pressure to afford the crude product. The crude was purified by reverse flash chromatography with the following conditions: column, C18 silica; mobile phase, MeCN in water, 10% to 50% gradient in 10 min; detector, UV 254 nm, hold for 20 min at 15%, after evaporated the solvent under reduced pressure to afford methyl 2-[4-(aminomethyl)phenyl]-3-[(tert-butoxycarbonyl)amino]propanoate I-57b (300 mg, 40.01% yield) as a white solid. LCMS: m/z (ESI), [2M+H]⁺=617.40.

Step 2: To a stirred mixture of amine I-57b (200 mg, 0.649 mmol, 1 equiv) and acetyl chloride (51.93 mg, 0.66 mmol, 1.02 equiv) in DCM (3.0 mL) was added Et₃N (196.89 mg, 1.95 mmol, 3 equiv) in portions at 0° C. under air atmosphere. The resulting mixture was stirred for 3 h at room temperature under air atmosphere. After reaction, the resulting mixture was diluted with water (10 mL). The resulting mixture was extracted with CH₂Cl₂ (3×20 mL). The combined organic layers were dried over anhydrous Na₂SO₄. After filtration, the filtrate was concentrated under reduced pressure. The residue was purified by prep-TLC with CH₂Cl₂/MeOH (30:1, v/v) to afford methyl 3-[(tert-butoxycarbonyl)amino]-2-[4-(acetamidomethyl)phenyl]propanoate I-57c (100 mg, 44.00% yield) as a white solid. LCMS: m/z (ESI), [M-Boc]⁺=251.15.

Step 3: To a stirred mixture of ester I-57c (150 mg, 0.43 mmol, 1 equiv) in MeOH (4.0 mL) was added LiOH·H₂O (71.85 mg, 1.71 mmol, 4 equiv) in water (1.0 mL) dropwise at room temperature under air atmosphere. The resulting mixture was stirred for overnight at 60° C. under air atmosphere. After cooling to room temperature, the resulting mixture was concentrated under reduced pressure. The resulting mixture was diluted with MeOH (2.0 mL). The residue was purified by reverse flash chromatography with the following conditions: column, C18 silica; mobile phase, MeCN in Water, 10% to 50% gradient in 10 min; detector, UV 254 nm, hold for 30 min at 26%. After evaporated solvent under reduced pressure to afford 3-[(tert-butoxycarbonyl)amino]-2-[4-(acetamidomethyl)phenyl]propanoic acid I-57 (92 mg, 63.89% yield) as a white solid. LCMS: m/z (ESI), [M+Na]⁺=359.00.

### Synthesis of Intermediate I-58

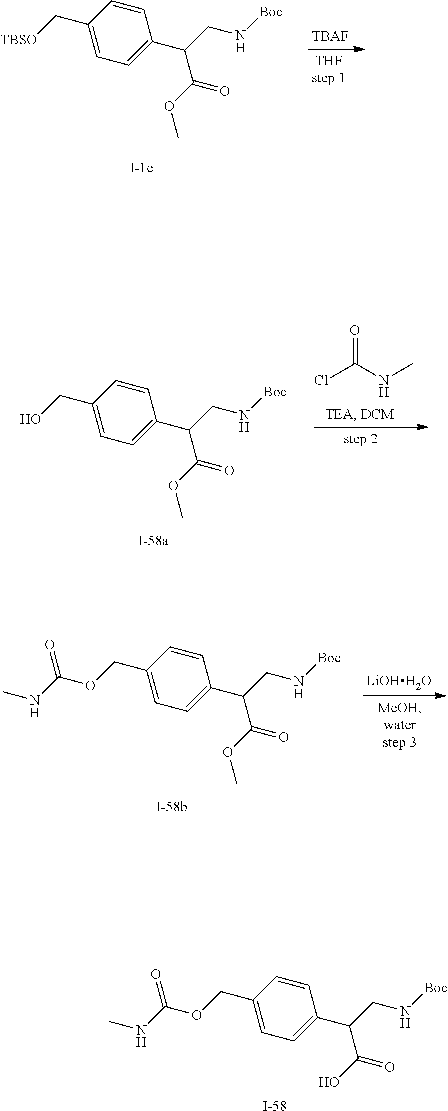

Step 1: Into a 40 mL vial were added methyl 3-[(tert-butoxycarbonyl)amino]-2-(4-{[(tert-butyldimethylsilyl)oxy]methyl}phenyl)propanoate I-1e (1.5 g, 3.54 mmol, 1 equiv) and TBAF (2.78 g, 10.62 mmol, 3 equiv) and THF (25 mL) at room temperature. The resulting mixture was stirred for 3 h at room temperature under air atmosphere. The reaction was quenched by the addition of water (50 mL) at room temperature. The aqueous layer was extracted with EtOAc (3×50 mL). The resulting mixture was concentrated under reduced pressure. The residue was purified by prep-TLC with CH₂Cl₂/EA (1:1, v/v) to afford methyl 3-[(tert-butoxycarbonyl)amino]-2-[4-(hydroxymethyl)phenyl]propanoate I-58a (975 mg, 89.01% yield) as a yellow oil. LCMS: m/z (ESI), [M+H]⁺=310.20. ¹H NMR (CD₃OD, 400 MHz) δ 1.42 (9H, s), 3.39 (1H, m), 3.64 (5H, m), 4.60 (2H, s), 5.51 (1H, s), 7.31 (4H, m).

Step 2: Into a 40 mL vial were added methyl 3-[(tert-butoxycarbonyl)amino]-2-[4-(hydroxymethyl)phenyl]propanoate (650 mg, 2.10 mmol, 1 equiv) and N-methylcarbamoyl chloride (432.24 mg, 4.62 mmol, 2.2 equiv) and TEA (1.06 g, 10.51 mmol, 5 equiv) and DCM (15 mL) at room temperature. The resulting mixture was stirred for 3 h at room temperature under air atmosphere. The resulting mixture was concentrated under reduced pressure. The residue was purified by prep-TLC with CH₂Cl₂/MeOH (40:1, v/v) to afford methyl 3-[(tert-butoxycarbonyl)amino]-2-(4-{[(methylcarbamoyl)oxy]methyl}phenyl)-propanoate I-58b (570 mg, 74.04% yield) as a yellow oil. LCMS: m/z (ESI), [M+H]⁺=367.05.

Step 3: Into a 100 mL round-bottom flask were added ester I-58b (570 mg, 1.56 mmol, 1.00 equiv) and LiOH·H₂O (163.18 mg, 3.89 mmol, 2.5 equiv) and MeOH (4 mL) and water (2 mL) at room temperature. The resulting mixture was stirred for 16 h at room temperature under air atmosphere. The resulting mixture was concentrated under reduced pressure. The residue was purified by reverse flash chromatography with the following conditions: column, C18 silica; mobile phase, MeOH in water (0.1% FA), 0% to 25% gradient in 20 min; detector, UV 254 nm. The resulting mixture was concentrated under reduced pressure to afford 3-[(tert-butoxycarbonyl)amino]-2-(4-{[(methylcarbamoyl)-oxy]methyl}phenyl)propanoic acid I-58 (520 mg, 94.86% yield) as an off-white solid. LCMS: m/z (ESI), [M+H-Boc]⁺=253.15.

### Synthesis of Intermediate I-59

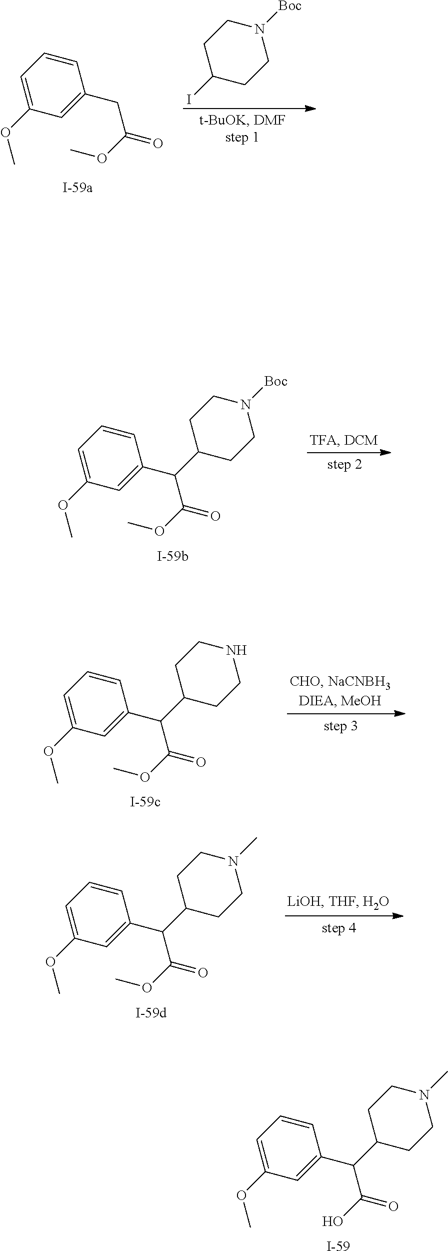

Step 1: Into a 50 mL round-bottom flask were added methyl 2-(3-methoxyphenyl)acetate I-59a (3 g, 16.65 mmol, 1 equiv), t-BuOK (3.74 g, 33.30 mmol, 2 equiv) and tert-butyl 4-iodopiperidine-1-carboxylate (6.73 g, 21.642 mmol, 1.3 equiv) in DMF (10 mL) at 0° C. The resulting mixture was stirred for 2 h at room temperature. The residue was purified by silica column chromatography, eluted with PE/EA (25:1, v/v) to afford piperidine I-59b (2.02 g, 33.38%) as a yellow oil. LCMS: m/z (ESI), [M+H−^{t}Bu]⁺=308.20. ¹H NMR (DMSO-d₆, 400 MHZ) δ 0.83-0.97 (1H, m), 1.04-1.17 (2H, m), 1.37 (9H, s), 1.65 (1H, dt), 2.07 (1H, qt), 2.66 (2H, d), 3.38 (1H, d), 3.59 (3H, s), 3.83 (1H, d), 3.90-3.98 (1H, m), 6.81-6.92 (3H, m), 7.19-7.30 (1H, m).

Step 2: Into a 50 mL round-bottom flask were added piperidine I-59b (2.0 g, 5.50 mmol, 1 equiv) and TFA (1.5 mL) in DCM (3 mL) at room temperature. The resulting mixture was stirred for 1.5 h at room temperature. The mixture was basified to pH 9 with saturated NaHCO₃ (aq.). The resulting mixture was extracted with DCM (3×50 mL). The combined organic layers were concentrated under reduced pressure to afford piperidine I-59c (1.3 g, 89.71%) as a yellow oil. LCMS: m/z (ESI), [M+H]⁺=264.20. ¹H NMR (DMSO-d₆, 400 MHz) δ 1.08-1.23 (1H, m), 1.23-1.33 (1H, m), 1.33-1.46 (1H, m), 1.81 (1H, dt), 2.22 (1H, qt), 2.77 (1H, td), 2.87 (1H, td), 3.15 (1H, dt), 3.26 (1H, dt), 3.43 (1H, d), 3.57 (3H, s), 3.75 (3H, s), 6.81-6.95 (3H, m), 7.28 (1H, t), 8.25 (2H, s).

Step 3: Into a 50 mL round-bottom flask were added piperidine I-59c (1.3 g, 4.94 mmol, 1 equiv), DIEA (3.19 g, 24.69 mmol, 5.0 equiv), sodium cyanoboranuide (620 mg, 9.87 mmol, 2 equiv) and CH₂O (296 mg, 9.88 mmol, 2 equiv) in MeOH (2.5 mL) at room temperature. The resulting mixture was stirred for overnight at room temperature. The reaction was quenched with water at room temperature. The resulting mixture was extracted with EtOAc (3×10 mL). The combined organic layers were concentrated under reduced pressure, to afford methylpiperidine I-59d (1.1 g, 80.34%) as a yellow oil. LCMS: m/z (ESI), [M+H]⁺=278.25.

Step 4: Into a 20 mL vial were added methylpiperidine I-59d (600 mg, 2.16 mmol, 1 equiv) in THF (2 mL) and LiOH (104 mg, 4.33 mmol, 2 equiv) in water (2 mL) at room temperature. The resulting mixture was stirred for 3 h at room temperature. The mixture acidified to pH 5 with HCl (1M). The residue was purified by reverse flash chromatography with the following conditions: column, C18 silica; mobile phase, water in MeOH, 0% to 30% gradient in 40 min; detector, UV 254 nm to afford acid I-59 (270 mg, 47.40%) as a white solid. LCMS: m/z (ESI), [M+H]⁺=264.20. ¹H NMR (DMSO-d₆, 400 MHZ) δ 0.95-1.16 (2H, m), 1.27 (1H, qd), 1.63-1.97 (4H, m), 2.14 (3H, s), 2.67 (1H, d), 2.79 (1H, d), 3.14 (1H, d), 3.73 (3H, s), 6.71-6.94 (3H, m), 7.23 (1H, t).

### Synthesis of Intermediate I-60

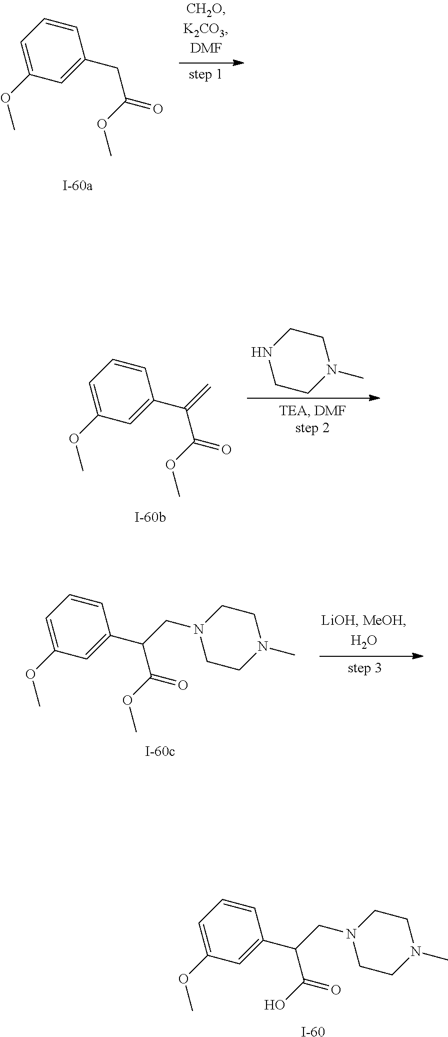

Step 1: Into a 20 mL vial were added ester I-60a (2 g, 11.10 mmol, 1 equiv), formaldehyde (0.67 g, 22.20 mmol, 2 equiv), K₂CO₃ (4.60 g, 33.30 mmol, 3 equiv) and DMF (2 mL) at room temperature. The resulting mixture was stirred for 1 h at 85° C. under. The mixture was allowed to cool down to room temperature. The resulting mixture was diluted with EtOAc (30 mL) and washed with water (3×30 mL), The combined organic layers were dried over anhydrous Na₂SO₄. After filtration, the filtrate was concentrated under reduced pressure. The residue was purified by prep-TLC with PE/EA (8:1, v/v) to afford enoate I-60b (1.3 g, 60.88%) as a light yellow oil. ¹H NMR (CD₃OD, 400 MHZ) δ 3.82 (6H, d), 5.95 (1H, d), 6.31 (1H, d), 6.88-6.95 (1H, m), 6.95-7.02 (2H, m), 7.23-7.31 (1H, m).

Step 2: Into a 100 mL round-bottom flask were added enolate I-60b (1.3 g, 6.76 mmol, 1 equiv), 1-methylpiperazine (2.71 g, 27.05 mmol, 4 equiv), TEA (2.05 g, 20.29 mmol, 3 equiv) and DMF (10 mL) at room temperature. The resulting mixture was stirred for 1 h at room temperature. The resulting mixture was diluted with EtOAc (50 mL) washed with 2×30 mL of water and 1×30 mL of saturated brine. The resulting mixture was concentrated under reduced pressure. This resulted in methylpiperidine I-60c (1.7 g, 81.33%) as a brown yellow oil. LCMS: m/z (ESI), [M+H]⁺=293.15.

Step 3. Into a 80 mL vial were added methylpiperidine I-60c (1 g, 3.42 mmol, 1 equiv), LiOH (163.83 mg, 6.84 mmol, 2 equiv), THF (5 mL) and water (5 mL) at room temperature. The resulting mixture was stirred for 1 h at room temperature. The mixture was acidified to pH 5 with HCl (1M). The residue was purified by reverse flash chromatography with the following conditions: column, C18 silica; mobile phase, MeOH in water, 10% to 50% gradient in 30 min; detector, UV 220 nm. This resulted in acid I-60 (497 mg, 51.16%) as a white solid. LCMS: m/z (ESI), [M+H]⁺=279.15.

### Synthesis of Compounds

### Example 1 (Racemic), Example 2 (Enantiomer 1), and Example 3 (enantiomer 2)

### Preparation of N-(3-(1H-pyrazol-4-yl)-1H-indol-7-yl)-3-amino-2-(4-(hydroxymethyl)phenyl)propanamide

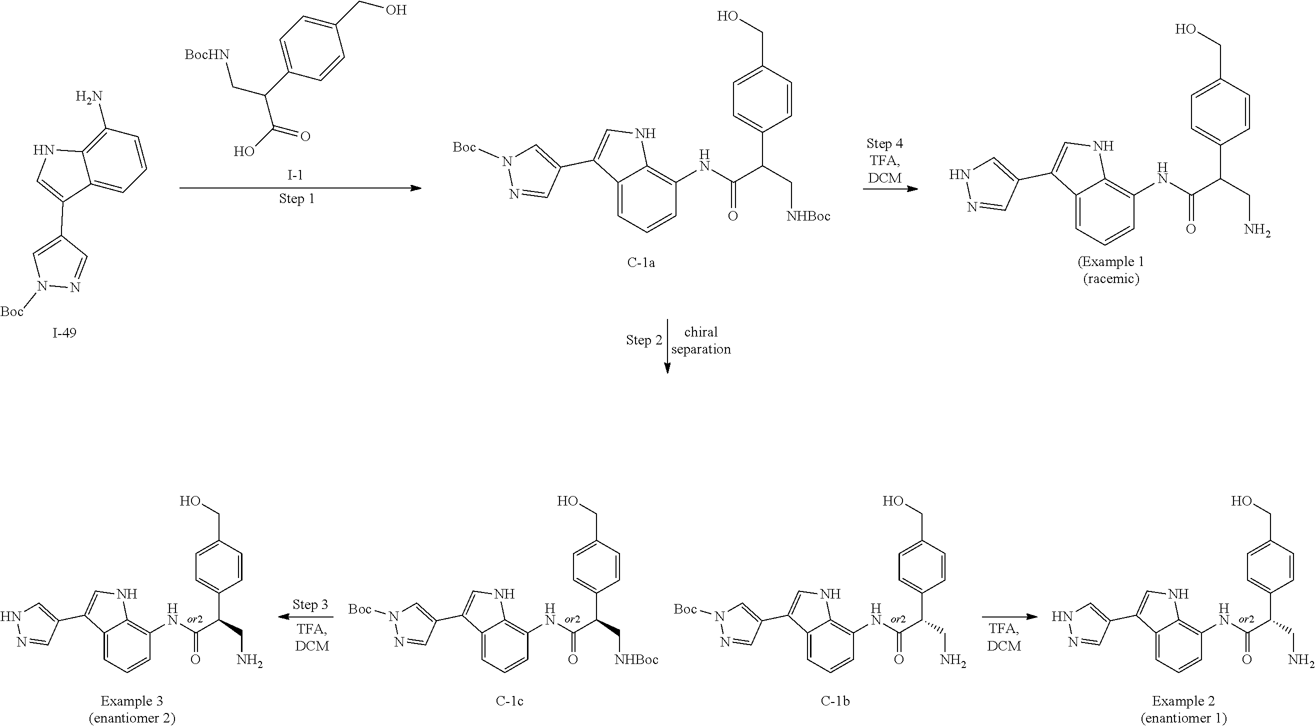

Step 1: A mixture of acid I-1 (498.91 mg, 1.69 mmol, 1.2 equiv) and amine I-49 (420 mg, 1.41 mmol, 1 equiv), HATU (802.92 mg, 2.11 mmol, 1.5 equiv), triethylamine (427.37 mg, 4.22 mmol, 3 equiv) in DMF (5 mL) was stirred for overnight at room temperature under nitrogen atmosphere. The resulting mixture was diluted with ethyl acetate (100 mL). The residue was washed with saturated NaHCO₃ (1×80 mL), water (2×80 mL), and saturated NaCl (1×80 mL). The residue was purified by reverse flash chromatography with the following conditions: column, C18 silica; mobile phase, MeCN in water, 35% to 65% gradient in 40 min; detector, UV 254 nm to afford amide C-1a (500 mg, 61.70%) as an off-white solid. LCMS: m/z (ESI), [M+H]⁺=576.5. ¹H NMR (DMSO-d₆, 400 MHz) δ 1.37 (9H, s), 1.62 (9H, s), 3.60 (1H, s), 4.08 (1H, s), 4.47 (2H, s), 5.13 (1H, s), 7.04 (1H, t), 7.10 (1H, s), 7.29 (2H, d), 7.38 (2H, d), 7.45 (1H, d), 7.60 (1H, d), 7.84 (1H, d), 8.26 (1H, s), 8.49 (1H, s), 9.97 (1H, s), 10.83 (1H, s).

Step 2: The amide C-1a (250 mg) was purified by prep-HPLC with the following conditions (Column: CHIRALPAK ID, 2*25 cm, 5 um; Mobile Phase A: Hex (0.2% DEA), Mobile Phase B: EtOH; Flow rate: 20 mL/min; Gradient: 50% B in 13 min; wavelength: 220/254 nm; sample solvent: EtOH) to afford enantiomer amide C-1b (80 mg, 32.00%) as an off-white solid. t_{R}=7.48 min. LCMS: m/z (ESI), [M+H]⁺=576.2. ¹H NMR (CD₃OD, 400 MHz) δ 1.44 (9H, s), 1.70 (9H, s), 3.48 (1H, dd), 3.79 (1H, dd), 4.11 (1H, dd), 4.62 (2H, s), 7.05-7.14 (2H, m), 7.38 (2H, d), 7.49 (2H, d), 7.64 (2H, q), 8.16 (1H, d), 8.42 (1H, d), SFC: t_{R}=1.41 min, ee=100%, and enantiomer C-1c (85.3 mg, 32.52%) as an off-white solid. t_{R}=9.78 min. LCMS: m/z (ESI), [M+H]⁺=576.2. ¹H NMR (CD₃OD, 400 MHZ) δ 1.44 (9H, s), 1.70 (9H, s), 3.48 (1H, dd), 3.79 (1H, dd), 4.11 (1H, dd), 4.62 (2H, s), 7.05-7.14 (2H, m), 7.38 (2H, d), 7.49 (2H, d), 7.64 (2H, q), 8.16 (1H, d), 8.42 (1H, d). SFC: t_{R}=1.89 min, ee=99.76%.

Step 3: (Example 3, enantiomer 2): To a solution of enantiomer C-1c (33 mg, 57.33 μmol) in DCM (1.48 mL) was cooled at 0° C. in ice/water bath, and stirred. Then added trifluoroacetic acid (261.45 mg, 2.29 mmol, 176.66 μL) dropwise into the reaction. The resulting mixture was stirred for 0.5 h. The reaction was concentrated under reduced pressure. The residue was purified by C18-flash chromatography column, elution gradient from 0% to 60% MeCN in water (6 mmol/L NH₄HCO₃). Pure fractions were lyophilized to dryness to afford amine Example 3, enantiomer 2 (20.76 mg, 93% yield) as white solid. LCMS: t_{R}=1.09 min in 3 min chromatography (5-95% MeCN in water (0.02% TFA), waters Acquity UPLC BEH C18 1.7 μm, 2.1*50 mm, 40° C.), MS (ESI) m/z=376.2 [M+H]⁺. ¹H NMR: (400 MHZ, DMSO-d₆) δ 12.80 (s, 1H), 10.83 (s, 1H), 7.94 (s, 2H), 7.57 (d, J=12.1 Hz, 2H), 7.49-7.31 (m, 3H), 7.29 (d, J=7.9 Hz, 2H), 6.98 (t, J=7.8 Hz, 1H), 5.14 (s, 1H), 4.46 (s, 2H), 3.92-3.84 (m, 1H), 2.89 (dd, J=11.7, 5.0 Hz, 1H).

Following the procedure above starting with amide C1-b, Example 2 (enantiomer 1) can be obtained.

Step 4: (Example 1, racemic): Into a 100 mL round-bottom flask were added amide C-1a (60 mg, 0.10 mmol, 1 equiv) in DCM (3 mL) and TFA (1 mL) at 0° C. The resulting mixture was stirred for 1 h at 0° C. under nitrogen atmosphere. The resulting mixture was concentrated under reduced pressure. The residue was dissolved in methanol (3 mL). The mixture was basified to pH 9 with NH₃ (aq). The crude product was purified by prep-HPLC with the following conditions (Column: Xselect CSH C18 OBD 30*150 mm; Mobile Phase A: water (0.1% FA), Mobile Phase B: ACN; Flow rate: 60 mL/min; Gradient: 5% B to 18% B in 7 min; wavelength: 254; 220 nm; t_{R}=6.05) to afford racemic Example 1 (21.5 mg, 54.94%) as a white solid. LCMS: m/z (ESI), [M+H]⁺=376.3. ¹H NMR (CD₃OD, 400 MHZ) δ 3.37 (1H, s), 3.68 (1H, dd), 4.26 (1H, dd), 4.65 (2H, s), 7.04-7.12 (2H, m), 7.43-7.55 (5H, m), 7.62-7.69 (1H, m), 7.92 (2H, s).

### Example 5 (Enantiomer 1) and Example 6 (Enantiomer 2)

### Preparation of 4-(1-((3-(1H-pyrazol-4-yl)-1H-indol-7-yl)amino)-3-amino-1-oxopropan-2-yl)benzyl 3,5-dimethylbenzoate

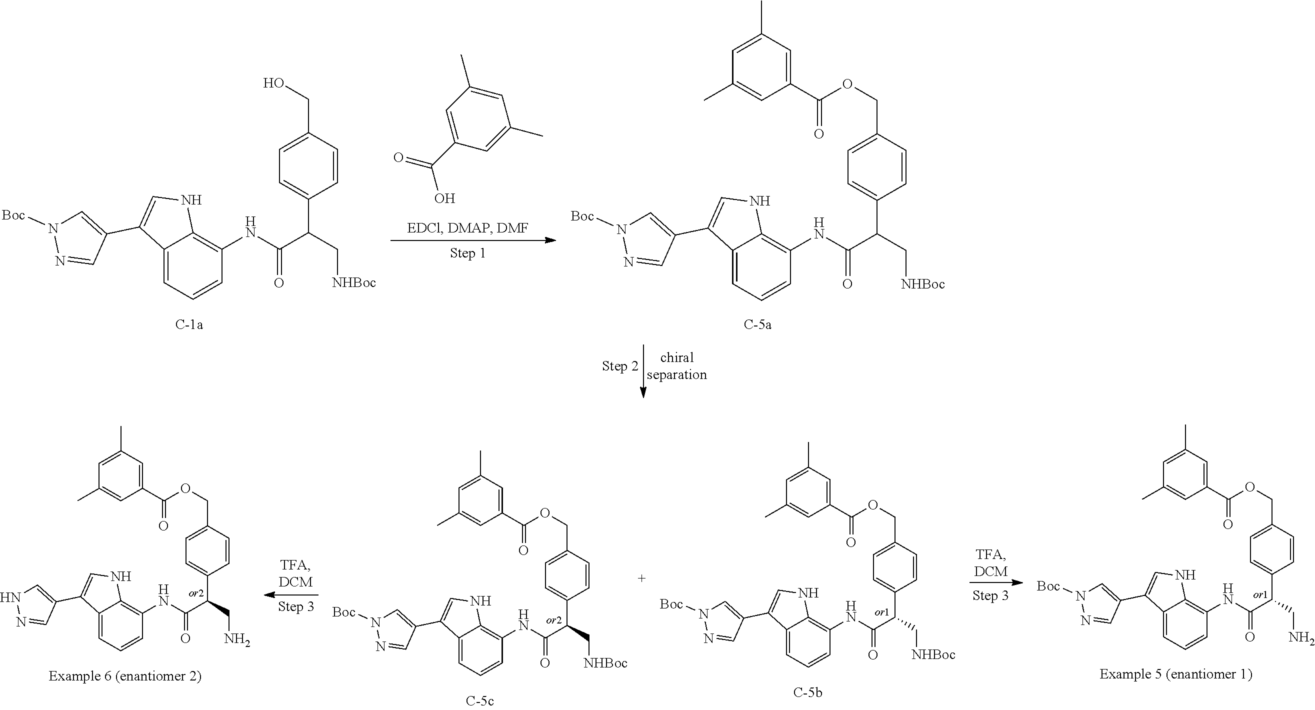

Step 1: Into a 50 mL round-bottom flask were added alcohol C-1a (146 mg, 0.25 mmol, 1 equiv) and 2,4-dimethylbenzoic acid (45.71 mg, 0.31 mmol, 1.2 equiv), EDCI (72.93 mg, 0.38 mmol, 1.5 equiv), DMAP (30.98 mg, 0.25 mmol, 1 equiv) in DMF (5 mL) at room temperature. The resulting mixture was stirred for 4 h at room temperature under nitrogen atmosphere. The product was precipitated by the addition of water. The crude product (146 mg) was purified by prep-HPLC with the following conditions (Column: XBridge Prep OBD C18 30*150 mm, 5 um; Mobile Phase A: water (10 mmol/L NH₄HCO₃), Mobile Phase B: ACN; Flow rate: 60 mL/min; Gradient: 55% B to 85% B in 7 min; wavelength: 220 nm; t_{R}=8.25 min) to afford racemic ester C-5a (130 mg, 72.42%) as an off-white solid. LCMS: m/z (ESI), [M+H]⁺=708.30. ¹H NMR (DMSO-d₆, 400 MHZ) δ 1.36 (9H, s), 1.62 (9H, s), 2.31 (3H, s), 2.50 (3H, s), 3.35-3.42 (1H, m), 3.61 (1H, dt), 4.12 (1H, t), 5.28 (2H, s), 6.97-7.16 (4H, m), 7.40-7.53 (5H, m), 7.61 (1H, d), 7.78 (1H, d), 7.84 (1H, d), 8.27 (1H, s), 8.49 (1H, s), 10.02 (1H, s), 10.83 (1H, s).

Step 2: The racemic ester C-5a (260 mg) was purified by prep-chiral HPLC with the following conditions (Column: (R, R) WHELK-O1, 4.6*50 mm, 3.5 um; Mobile Phase A: Hex (0.2% DEA): (EtOH/DCM=1:1, v/v)=65:35 (v/v); Flow rate: 1 mL/min; Injection Volume: 5 ul mL) to afford enantiomer ester C-5c, SFC: t_{R}=2.79 min, ee=100%, and enantiomer ester C-5b, SFC: t_{R}=3.567 min, ee=100%.

Step 3: Into a 100 mL vial were added enantiomer ester C-5c (110 mg, 0.16 mmol, 1 equiv) in DCM (3 mL) and TFA (1 mL) at room temperature. The resulting mixture was stirred for 30 min at room temperature under nitrogen atmosphere. The resulting mixture was concentrated under reduced pressure. The residue was dissolved in methanol (3 mL). The residue was basified to pH 8 with NH₃ aq. The resulting mixture was stirred for 1 h at 0° C. under nitrogen atmosphere. The crude product (80 mg) was purified by prep-HPLC with the following conditions (Column: XBridge Shield RP18 OBD 30*150 mm, 5 um; Mobile Phase A: water (10 mmol/L NH₄HCO₃), Mobile Phase B: ACN; Flow rate: 60 mL/min; Gradient: 30% B to 60% B in 8 min; wavelength: 220 nm; t_{R}=7.58 min) to afford Example 6, enantiomer 2 (28.8 mg, 36.51%) as a white solid. LCMS: m/z (ESI), [M+H]⁺=508.25. ¹H NMR (CD₃OD, 400 MHZ) δ 2.35 (3H, s), 2.54 (3H, s), 3.08 (1H, dd), 3.46 (1H, dd), 4.01 (1H, dd), 5.33 (2H, s), 6.98-7.08 (2H, m), 7.08-7.15 (2H, m), 7.45 (1H, s), 7.48-7.57 (4H, m), 7.64 (1H, dd), 7.83 (1H, d), 7.91 (2H, s). SFC: t_{R}=2.338 min, ee=99.28%.

Into a 100 mL vial were added enantiomer ester C-5b (110 mg, 0.16 mmol, 1 equiv) in DCM (3 mL) and TFA (1 mL) at room temperature. The resulting mixture was stirred for 30 min at room temperature under nitrogen atmosphere. The resulting mixture was concentrated under reduced pressure. The residue was dissolved in methanol (3 mL). The residue was basified to pH 8 with NH₃ aq. The resulting mixture was stirred for 1 h at 0° C. under nitrogen atmosphere. The crude product was purified by prep-HPLC with the following conditions (Column: XBridge Prep OBD C18 30*150 mm, 5 um; Mobile Phase A: water (10 mmol/L NH₄HCO₃), Mobile Phase B: ACN; Flow rate: 60 mL/min; Gradient: 34% B to 54% B in 8 min; wavelength: 254 nm; t_{R}=7.83 min) to afford Example 5, enantiomer 1 (24.5 mg, 42.71%) as a white solid. LCMS: m/z (ESI), [M+H]⁺=508.25. ¹H NMR (CD₃OD, 400 MHZ) δ 2.35 (3H, s), 2.55 (3H, s), 3.04 (1H, dd), 3.43 (1H, dd), 3.96 (1H, dd), 5.34 (2H, s), 7.03-7.15 (4H, m), 7.42-7.54 (5H, m), 7.61-7.85 (2H, m), 7.91 (2H, s). SFC: t_{R}=2.123 min, ee=100%.

### Example 7 (Racemic)

### Preparation of N-(3-(1H-pyrazol-4-yl)-1H-indol-7-yl)-2-amino-2-phenylacetamide (racemic)

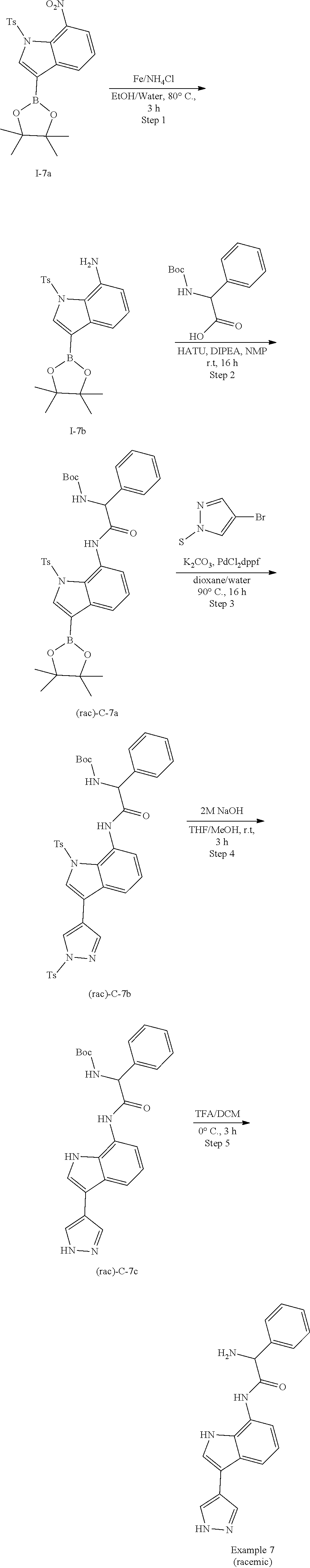

Step 1: To a mixture of nitro-indole I-7a (5 g, 11.30 mmol) and ammonia hydrochloride (6.05 g, 113.05 mmol) in EtOH (30 mL) and water (30 mL) was added iron powder (3.16 g, 56.52 mmol, 401.63 μL). Then the reaction mixture was stirred and heated at 80 CC for 3 h. The reaction mixture was filtered through celite and washed with ethanol (50 mL×3), and the filtrate was concentrated. The residue was dissolved into 200 ml of ethyl acetate, filtered and the filtrate was concentrated under reduced pressure to afford amine I-7b (3.10 g, 67% yield) as brown solid. LCMS: t_{R}=2.07 min in 3 min chromatography (3 min-5-95% MeCN in water (0.02% TFA), Waters Acquity UPLC BEH C18 1.7 μm, 2.1*50 mm, 40° C.), MS (ESI) m/z=413.2 [M+H]⁺.

Step 2: amine I-7b (3.1 g, 7.52 mmol), 2-(tert-butoxycarbonylamino)-2-phenyl-acetic acid (3.21 g, 12.78 mmol) and HATU (4.86 g, 12.78 mmol) in NMP (20 mL) were stirred at room temperature for 15 min. Then DIPEA (4.37 g, 33.83 mmol, 5.89 mL) was added at room temperature. The resulting suspension was stirred at room temperature for 16 h. The reaction mixture was diluted with 200 ml of water, then stirred at room temperature for 30 min., filtered and washed with water (10 mL×2). The solid was collected and dried to afford amide racemic C-7a (1.60 g, 33% yield) as brown solid. LCMS: t_{R}=2.13 min in 3 min chromatography (3 min-5-95% MeCN in water (0.02% TFA), Waters Acquity UPLC BEH C18 1.7 μm, 2.1*50 mm, 40° C.), MS (ESI) m/z=646.7 [M+H]⁺.

Step 3: racemic C-7a (200 mg, 309.80 μmol), 4-bromo-1-tosyl-1H-pyrazole (139.95 mg, 464.70 μmol), K₂CO₃ (128.45 mg, 929.41 μmol) and PdCl₂dppf (25.30 mg, 30.98 μmol) in dioxane (3.00 mL) and water (0.60 mL) were stirred under nitrogen at 90° C. for 16 h. The solvent was removed under reduced pressure. The residue was purified by silica flash chromatography column, elution gradient from 0 to 40% ethyl acetate in petroleum ether. Pure fractions were evaporated to dryness to afford racemic amide C-7b (136 mg, 59% yield) as brown solid. LCMS: t_{R}=2.01 min in 3 min chromatography (3 min-5-95% MeCN in water (0.02% TFA), Waters Acquity UPLC BEH C18 1.7 μm, 2.1*50 mm, 40° C.), MS (ESI) m/z=740.7 [M+H]⁺.

Step 4 and Step 5: racemic amide C-7b (136 mg, 183.82 μmol) was inserted into THF (0.60 mL)/MeOH (0.60 mL), after which 2N of sodium hydroxide (73.53 mg, 1.84 mmol, 34.52 μL) in water was added into the reaction. The reaction mixture was stirred at room temperature for 3 h. When the reaction was completed, the mixture was stirred and cooled down to 0° C. by ice/water bath, then saturated NH₄Cl aqueous solution (20 mL) was added and extracted with dichloromethane (10 mL×3) and the combined organic layer was washed with brine (50 mL×1), dried over anhydrous sodium sulfate, filtered and concentrated under reduced pressure to afford crude racemic amide C-7c, which was dissolved into DCM (2.00 mL). The reaction was stirred and cooled at 0° C. by ice/water bath. Then added trifluoroacetic acid (1.05 g, 9.19 mmol, 708.08 μL) dropwise into the reaction. The resulting mixture was stirred at 0° C. for 2 h. Then resulting mixture was concentrated under reduced pressure. The residue was purified by C18-flash chromatography, elution gradient from 0% to 30% MeCN in water (0.02% TFA). Pure fractions were lyophilized to dryness to afford racemic Example 7 (3.5 mg, 7.88 μmol, 4.28% yield, TFA salt) as brown solid. LCMS: t_{R}=0.93 min in 3 min chromatography (3 min-5-95% MeCN in water (0.02% TFA), Waters Acquity UPLC BEH C18 1.7 μm, 2.1*50 mm, 40° C.), MS (ESI) m/z=332.4 [M+H]⁺. ¹H NMR: (400 MHZ, DMSO-d₆) δ 8.42 (s, 2H), 8.36 (d, J=7.7 Hz, 1H), 8.29-8.25 (m, 1H), 8.16 (dd, J=7.5, 1.9 Hz, 1H), 8.10 (d, J=7.8 Hz, 1H), 7.96-7.84 (m, 5H), 7.60 (dd, J=7.4, 1.0 Hz, 1H), 7.46 (t, J=7.4 Hz, 1H), 6.35 (s, 1H).

### Example 8

### Preparation of (2R)-2-amino-2-phenyl-N-[3-(1H-pyrazol-4-yl)-1H-indol-7-yl]acetamide

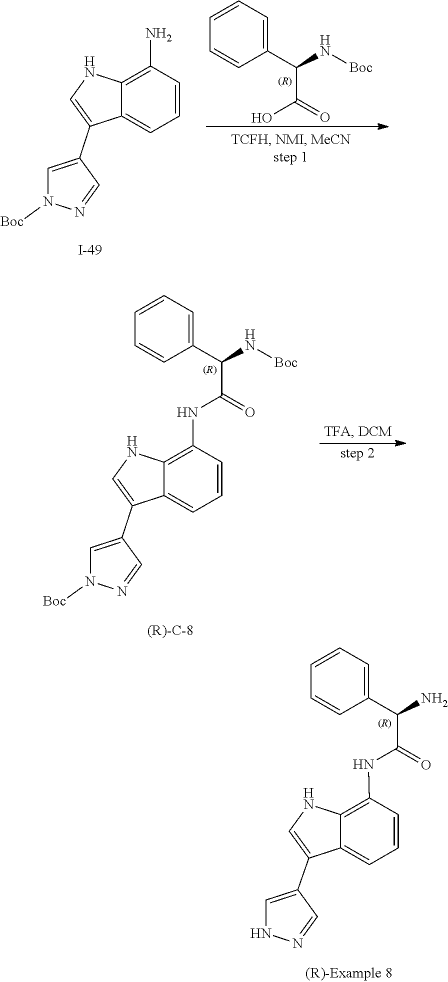

Step 1: Into a 80 mL vial were added amine I-49 (120 mg, 0.40 mmol, 1 equiv), (R)-[(tert-butoxycarbonyl)amino](phenyl) acetic acid (151.61 mg, 0.60 mmol, 1.5 equiv), TCFH (225.71 mg, 0.80 mmol, 2 equiv), NMI (165.12 mg, 2.01 mmol, 5 equiv) and MeCN (5 mL) at room temperature. The resulting mixture was stirred for 8 h at room temperature. The resulting mixture was concentrated under reduced pressure. The residue was purified by prep-TLC with CH₂Cl₂/methanol (40:1, v/v) to afford amide (R)-C-8 (160 mg, 83.39%) as a yellow solid. LCMS: m/z (ESI), [M+H−^{t}Bu]⁺=476.20.

Step 2: Into a 50 mL round-bottom flask were added amide (R)-C-8 (120 mg, 0.23 mmol, 1 equiv), TFA (1 mL) and DCM (3 mL) at room temperature. The resulting mixture was stirred for 1 h at room temperature. The resulting mixture was diluted with CH₂Cl₂ (20 mL). The mixture was allowed to cool down to 0° C. The mixture basified to pH 9 with NH₃ aq. The crude product (100 mg) was purified by prep-HPLC with the following conditions (Column: XBridge Shield RP18 OBD 30*150 mm, 5 um; Mobile Phase A: water (10 mmol/L NH₄HCO₃+0.1% NH₃ aq), Mobile Phase B: ACN; Flow rate: 60 mL/min; Gradient: 17% B to 40% B in 8 min; wavelength: 220 nm; t_{R}=7.67 min). The crude product (60 mg) was purified by prep-chiral-HPLC with the following conditions (Column: CHIRAL ART Cellulose-SB, 2*25 cm, 5 um; Mobile Phase A: Hex (0.2% DEA), Mobile Phase B: EtOH/DCM (1:1, v/v); Flow rate: 20 mL/min; Gradient: 45% B to 45% B in 16 min; wavelength: 220/254 nm; t_{R}-1=8.97 min; t_{R}-2=12.51 min; Sample Solvent: EtOH/DCM (1:1, v/v); Injection Volume: 0.7 mL). The crude product (32 mg) was purified by prep-HPLC with the following conditions (Column: XBridge Shield RP18 OBD 30*150 mm, 5 um; Mobile Phase A: water (10 mmol/L NH₄HCO₃+0.1% NH₃ aq), Mobile Phase B: ACN; Flow rate: 60 mL/min; Gradient: 17% B to 40% B in 8 min, 40% B; wavelength: 220 nm; t_{R}=7.67 min) to afford (R)-Example 8 (17.5 mg, 23.34%) as a white solid. LCMS: m/z (ESI), [M+H]⁺=332.10. ¹H NMR (CD₃OD, 400 MHZ) δ 4.76 (1H, s), 7.07 (1H, t), 7.14 (1H, dd), 7.31-7.39 (1H, m), 7.39-7.48 (3H, m), 7.59 (2H, dd), 7.65 (1H, dd), 7.92 (2H, s). SFC: t_{R}=0.938 min, ee=100%.

### Example 9

### Preparation of (2S)-2-amino-2-phenyl-N-[3-(1H-pyrazol-4-yl)-1H-indol-7-yl]acetamide

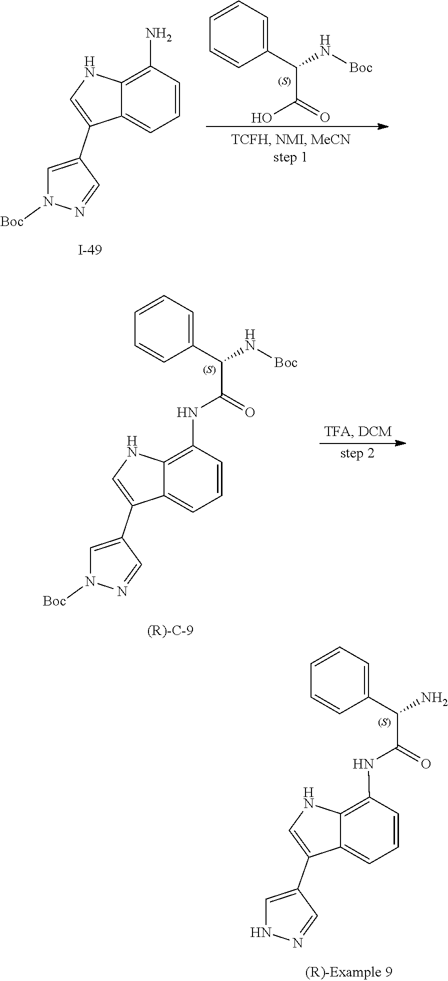

Step 1: A solution of amine I-49 (150 mg, 0.50 mmol, 1 equiv) and(S)-[(tert-butoxycarbonyl)amino](phenyl) acetic acid (189.51 mg, 0.75 mmol, 1.5 equiv), TCFH (211.60 mg, 0.75 mmol, 1.5 equiv), NMI (144.48 mg, 1.76 mmol, 3.5 equiv) in MeCN (3 mL) was stirred for 1 h at room temperature under nitrogen atmosphere. The residue was purified by prep-TLC with CH₂Cl₂/methanol (22:1, v/v) to afford amide(S)-C-9 (200 mg, 74.83%) as a brown yellow solid. LCMS: m/z (ESI), [M+H−^{t}Bu]⁺=476.25.

Step 2: A solution of amide(S)-C-9 (160 mg, 0.27 mmol, 1 equiv) and TFA (1.00 mL) in DCM (3 mL) were stirred for 1 h at room temperature under nitrogen atmosphere. The resulting mixture was concentrated under reduced pressure. The resulting mixture was diluted with methanol (2 mL). The mixture was basified to pH 9 with aqueous ammonia at 0° C., and stirred for 30 min. The crude product (200 mg) was purified by prep-HPLC with the following conditions (Column: XBridge Shield RP18 OBD 30*150 mm, 5 um; Mobile Phase A: water (10 mmol/L NH₄HCO₃+0.1% NH₃ aq), Mobile Phase B: ACN; Flow rate: 60 mL/min; Gradient: 17% B to 40% B in 8 min; wavelength: 220 nm; t_{R}=7.77 min) to afford(S)-Example 9 (46.6 mg, 20.74%) as an off-white solid. LCMS: m/z (ESI), [M+H]⁺=332.15. ¹H NMR (CD₃OD, 400 MHZ) δ 4.75 (1H, s), 7.07 (1H, t), 7.14 (1H, d), 7.31-7.39 (1H, m), 7.43 (3H, dd), 7.59 (2H, d), 7.65 (1H, d), 7.91 (2H, s). SFC: t_{R}=1.364 min, ee=100%.

### Example 10 (Enantiomer 1)

### Preparation of [4-[(1S)-1-(aminomethyl)-2-oxo-2-[[3-(1H-pyrazol-4-yl)-1H-indol-7-yl]amino]ethyl]phenyl]methyl 6-nitrooxyhexanoate

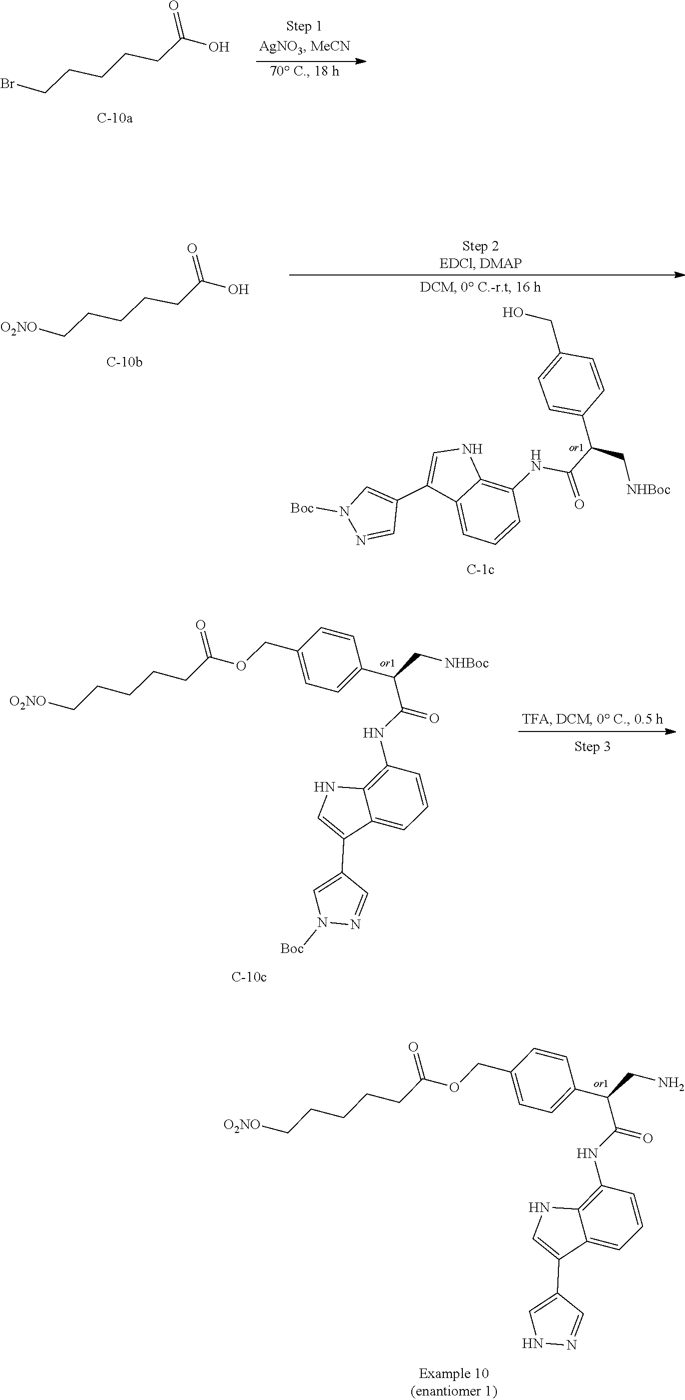

Step 1:6-bromohexanoic acid C-10a (500 mg, 2.56 mmol) and silver nitrate (566.09 mg, 3.33 mmol, 130.08 μL) in MeCN (12.5 mL) at 70° C. for 18 h. Then the reaction solution was evaporated under reduced pressure. Then added 10 mL of DCM. The suspension was stirred at room temperature for 15 min filtered and evaporated under reduced pressure to afford 6-nitrooxyhexanoic acid C-10b (440 mg, 97% yield) as brown oil. ¹H NMR: (400 MHz, CDCl₃) δ 11.18 (s, 1H), 4.45 (t, J=6.6 Hz, 2H), 2.38 (t, J=7.3 Hz, 2H), 1.83-1.63 (m, 4H), 1.56-1.40 (m, 2H).

Step 2:6-nitrooxyhexanoic acid C-10b (23.08 mg, 130.29 μmol) was dissolved into DCM (1.50 mL) at 0° C. by ice/water bath. Added EDCI (24.98 mg, 130.29 μmol), DMAP (1.06 mg, 8.69 μmol), and enantiomer alcohol C-1c (50 mg, 86.86 μmol). The reaction mixture was stirred and warmed slowly to 25° C. for 16 h. The resulting mixture was diluted with water (50 mL), extracted with ethyl acetate (20 mL×3). The combined organic layer was washed with brine (60 mL×1), dried over anhydrous sodium sulfate, filtered and evaporated under reduced pressure to afford enantiomer ester C-10c (62 mg, 97% yield) as brown solid. LCMS: t_{R}=2.07 min in 3 min chromatography (5-95% MeCN in water (6 mmol/L NH₄HCO₃), waters Acquity UPLC BEH C18 1.7 μm, 2.1*50 mm, 40° C.), MS (ESI) m/z=735.2 [M+H]⁺.

Step 3: To a solution of enantiomer C-10c (62.00 mg, 84.38 μmol) in DCM (3.01 mL) was cooled at 0° C. in ice/water bath, and stirred. Then added TFA (481.05 mg, 4.22 mmol, 325.03 μL) dropwise into the reaction. The resulting mixture was stirred for 2 h. The reaction mixture was concentrated under reduced pressure. The residue was purified by C18-flash chromatography, elution gradient from 0% to 50% MeCN in water (6 mmol/L NH₄HCO₃). Pure fractions were lyophilized to dryness to afford nitrooxy ester Example 10, enantiomer 1 (40 mg, 89% yield) as white solid. LCMS: t_{R}=1.63 min in 3 min chromatography (5-95% MeCN in water (6 mmol/L NH₄HCO₃), Waters Acquity UPLC BEH C18 1.7 μm, 2.1*50 mm, 40° C.), MS (ESI) m/z=535.3 [M+H]⁺. ¹H NMR: (500 MHZ, DMSO-d₆) δ 12.85 (s, 1H), 10.81 (s, 1H), 7.95 (s, 2H), 7.57 (d, J=8.8 Hz, 2H), 7.51-7.20 (m, 5H), 6.98 (t, J=7.5 Hz, 1H), 5.06 (s, 2H), 4.48 (t, J=6.2 Hz, 2H), 3.98-3.84 (m, 1H), 3.29-3.26 (m, 1H), 2.98-2.84 (m, 1H), 2.35 (t, J=7.0 Hz, 2H), 1.75-1.47 (m, 4H), 1.42-1.27 (m, 2H).

### Example 11 (Enantiomer 1)

### Preparation of 4-(1-((3-(1H-pyrazol-4-yl)-1H-indol-7-yl)amino)-3-amino-1-oxopropan-2-yl)benzyl 5-(nitrooxy) pentanoate

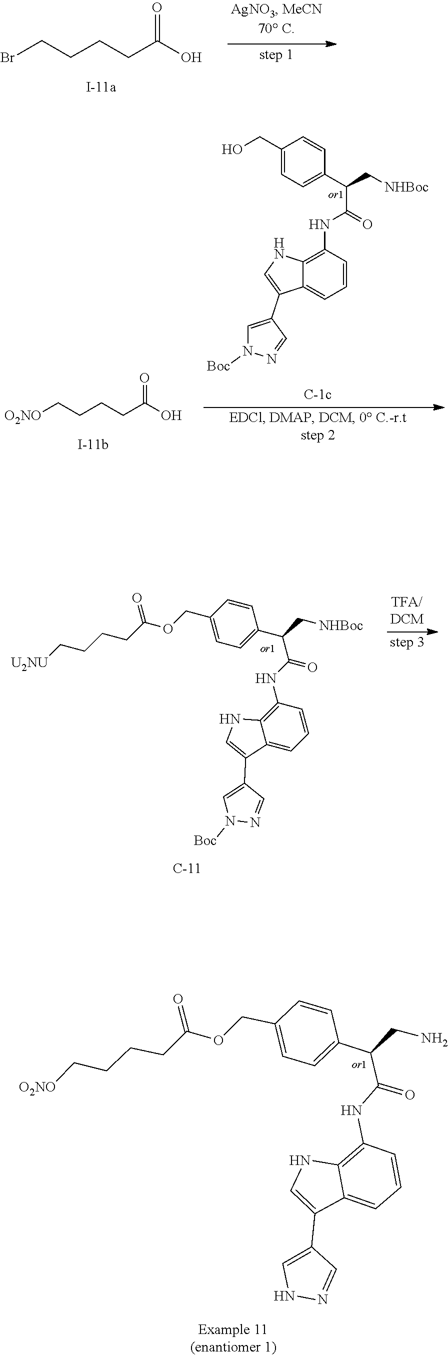

Step 1: A mixture of 5-bromopentanoic acid I-11a (1 g, 5.52 mmol, 1 equiv) and AgNO₃ (1.9 g, 11.05 mmol, 2 equiv) in MeCN (15 mL) was stirred for 24 h at 70° C. under nitrogen atmosphere. The reaction was light sensitive and the light should be avoided. The mixture was cooled to room temperature. The precipitated solids were collected by filtration and washed with dichloromethane (5×40 mL). After filtration, the filtrate was concentrated under reduced pressure to give 600 mg of 5-(nitrooxy) pentanoic acid I-11b as a colorless oil (67%). ¹H NMR (DMSO-d₆, 400 MHz) δ 1.58-1.63 (2H, m), 1.79-1.81 (2H, m), 2.23-2.29 (2H, m), 3.49-3.54 (2H, m), 12.08 (1H, s).

Step 2: To a mixture of 5-(nitrooxy) pentanoic acid I-11b (120 mg, 0.74 mmol, 5 equiv) in DCM (10 mL) were added EDCI (57 mg, 0.30 mmol, 2 equiv) and DMAP (2 mg, 0.016 mmol, 0.1 equiv) for 30 min at 0° C. under nitrogen atmosphere followed by the addition of alcohol C-1c (85 mg, 0.15 mmol, 1 equiv) dropwise at 0° C. The reaction was stirred for 16 h after the mixture warmed to room temperature. The reaction was quenched with water (20 mL) at room temperature and extracted with CH₂Cl₂ (3×20 mL). The combined organic layers were washed with sat. NaCl (3×20 mL), dried over anhydrous Na₂SO₄. After filtration, the filtrate was concentrated under reduced pressure. The residue was purified by prep-TLC eluted with dichloromethane/methanol (30:1, v/v) to give 60 mg of enantiomer C-11 as an orange solid (40%). LCMS: m/z (ESI), [M+H]⁺=721.31.

Step 3: A mixture of enantiomer C-11 (60 mg, 0.08 mmol, 1 equiv) and TFA (0.8 mL, 10.74 mmol) in DCM (3 mL) was stirred for 30 min at 0° C. under nitrogen atmosphere. The resulting mixture was concentrated under reduced pressure. The residue was neutralized to pH 7˜8 with sat. NaHCO₃ and extracted with CH₂Cl₂ (3×10 mL). The combined organic layers were washed with sat. NaCl (3×10 mL), dried over anhydrous Na₂SO₄. After filtration, the filtrate was concentrated under reduced pressure. The residue was purified by prep-HPLC with the following conditions (The mobile phase consisted of a mixture of solvent 0.1% NH₄HCO₃ in water and 0.1% NH₄OH in acetonitrile. A constant gradient from 70% aqueous/30% organic to 47% aqueous/53% organic mobile phase over the course of 8 minutes was utilized. The flow rate was constant at 60 mL/min.) to give 10.6 mg of Example 11, enantiomer 1 as an orange solid (24%). LCMS: m/z (ESI), [M+H]⁺=521.25. ¹H NMR (DMSO-d₆, 400 MHz) δ 1.60-1.69 (4H, m), 2.39 (2H, t), 2.88-2.93 (1H, m), 3.85-3.92 (1H, m), 4.50 (2H, t), 5.07 (2H, s), 6.96 (1H, t), 7.32-7.38 (3H, m), 7.43 (2H, d), 7.56-7.60 (2H, m), 7.96 (2H, d), 7.82-8.12 (2H, m), 10.78 (1H, s), 12.81-12.88 (1H, m).

### Example 12 (Enantiomer 1)

### Preparation of 4-(1-((3-(1H-pyrazol-4-yl)-1H-indol-7-yl)amino)-3-amino-1-oxopropan-2-yl)benzyl 4-(nitrooxy) butanoate

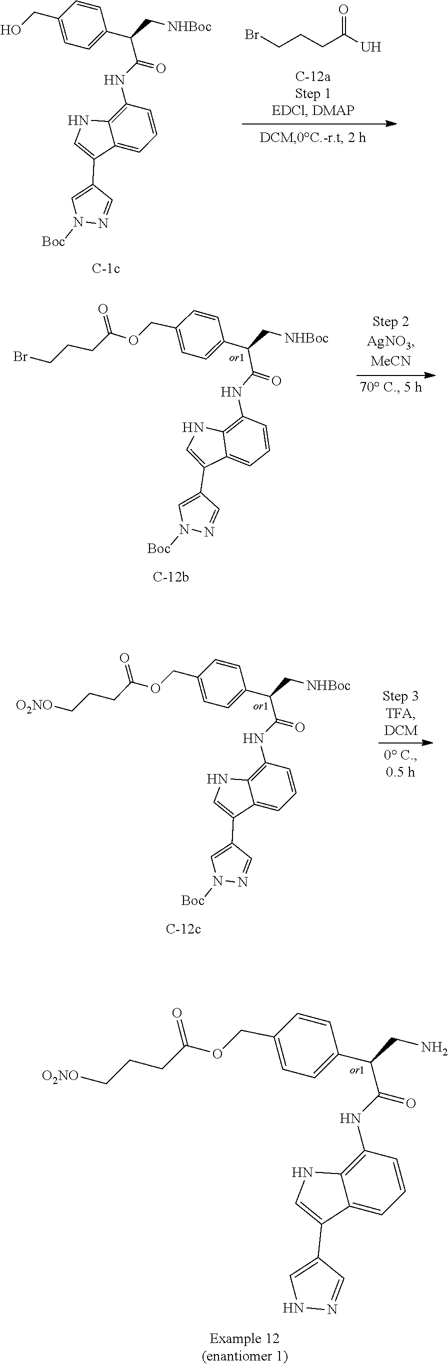

Step 1: To a mixture of 4-bromobutanoic acid C-12a (290 mg, 1.75 mmol, 5 equiv) in DCM (30 mL) were added EDCI (133 mg, 0.70 mmol, 2 equiv) and DMAP (4.2 mg, 0.04 mmol, 0.1 equiv) for 30 min at 0° C. under nitrogen atmosphere followed by the addition of alcohol C-1c (200 mg, 0.35 mmol, 1 equiv). The reaction was stirred for 2 h at room temperature. The mixture was quenched with water (30 mL), extracted with CH₂Cl₂ (3×30 mL), washed with sat. NaCl (3×20 mL) and dried over anhydrous sodium sulfate. After filtration, the filtrate was concentrated under reduced pressure. The residue was purified by prep-TLC eluted with dichloromethane/methanol (30/1, v/v) to afford 1H-indol-3-yl)-1H-bromoester C-12b as a yellow solid (220 mg, 88% yield). LCMS: m/z (ESI), [M+H]⁺=724.23.

Step 2: A mixture of bromoester C-12b (120 mg, 0.17 mmol, 1 equiv) and AgNO₃ (56 mg, 0.34 mmol, 2 equiv) in MeCN (15 mL) was stirred for 2 h at 70° C. under nitrogen atmosphere. The reaction was light sensitive and the light should be avoided. The mixture was cooled to room temperature. The resulting mixture was filtered and the filter cake was washed with CH₂Cl₂ (3×50 mL) and concentrated under reduced pressure to afford nitrooxy ester C-12c as an orange solid (25 mg, 26% yield). LCMS: m/z (ESI), [M+H]⁺=707.2.

Step 3: A mixture of nitrooxy ester C-12c (25 mg, 0.04 mmol, 1 equiv) and TFA (0.6 mL, 8 mmol) in DCM (3 mL) was stirred for 30 min at 0° C. under nitrogen atmosphere. The mixture was neutralized to pH 7˜8 with ammonia and concentrated under reduced pressure. The residue was further purified by prep-HPLC with the following conditions (The mobile phase consisted of a mixture of solvent 0.1% formic acid in water and acetonitrile. A constant gradient from 83% aqueous/17% organic to 70% aqueous/30% organic mobile phase over the course of 7 minutes was utilized. The flow rate was constant at 70 mL/min.) to afford Example 12, enantiomer 1 as a white solid (2 mg, 9% yield). LCMS: m/z (ESI), [M+H]⁺=507.35. ¹H NMR: (DMSO-d₆, 400 MHz) δ 1.92-1.96 (2H, m), 2.47-2.50 (2H, m), 3.03-3.07 (2H, m), 4.07-4.11 (1H, m), 4.54 (2H, t), 5.09 (2H, s), 6.97-7.01 (1H, m), 7.37 (3H, m), 7.46 (2H, d), 7.59 (2H, d), 7.95 (2H, s), 8.28 (1H, s), 10.08 (1H, br s), 10.83 (1H, s).

### Example 14

### Preparation of (R)-N-(3-(1H-pyrazol-4-yl)-1H-indol-7-yl)-3-amino-2-phenylpropanamide

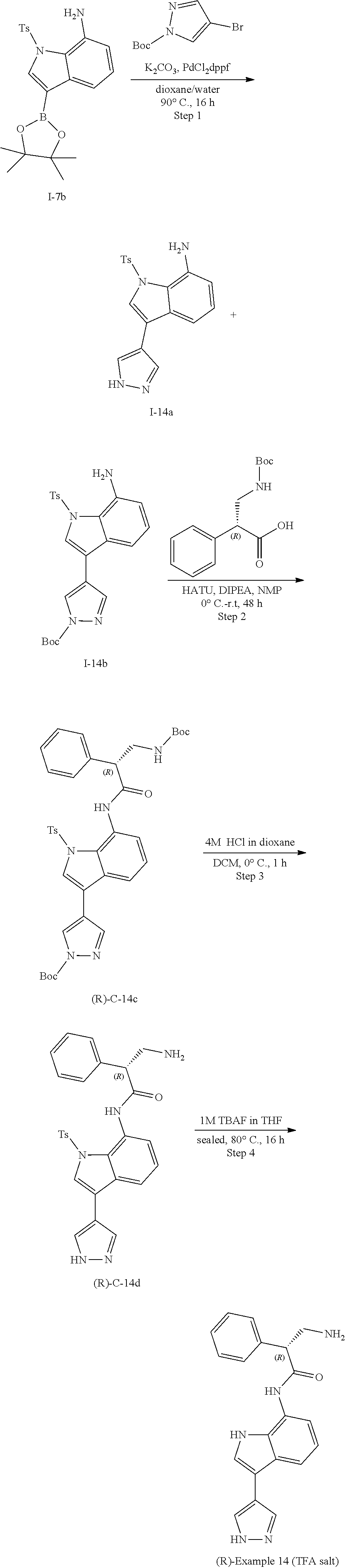

Step 1: amine I-7b (3.560 g, 8.63 mmol), tert-butyl 4-bromopyrazole-1-carboxylate (3.20 g, 12.95 mmol), potassium carbonate (3.58 g, 25.90 mmol) and PdCl₂dppf (705.11 mg, 863.43 μmol) in dioxane (60.00 mL) and water (12.00 mL) were stirred under nitrogen at 90° C. for 16 h. The solvent was removed under reduced pressure. The residue was purified by silica chromatography column, elution gradient from 10 to 50% ethyl acetate in petroleum ether. Pure fractions were evaporated to dryness to afford amine I-14b (0.471 g, 748.56 μmol, 9% yield, 72% purity). LCMS: t_{R}=1.77 min in 3 min chromatography (3 min-5-95% MeCN in water (0.02% TFA), Waters Acquity UPLC BEH C18 1.7 μm, 2.1*50 mm, 40° C.), MS (ESI) m/z=453.6 [M+H]⁺ and amine I-14a (3.19 g, 6.88 mmol, 80% yield, 76% purity) as brown solid. LCMS: t_{R}=1.43 min in 3 min chromatography (3 min-5-95% MeCN in water (0.02% TFA), Waters Acquity UPLC BEH C18 1.7 μm, 2.1*50 mm, 40° C.), MS (ESI) m/z=353.6 [M+H]⁺.

Step 2: To a stirred mixture of amine I-14b (85 mg, 135.09 μmol), (R)-3-((tert-butoxycarbonyl)amino)-2-phenylpropanoic acid (39.42 mg, 148.60 μmol) and DIPEA (26.19 mg, 202.64 μmol, 35.29 μL) in NMP (2.00 mL) was cooled and stirred at 0° C. Then HATU (56.80 mg, 149.39 μmol) was added in one portion at this temperature. The resulting mixture was stirred at room temperature for 17 h. Then detected the reaction by LCMS, the reaction was incomplete. More (R)-3-((tert-butoxycarbonyl)amino)-2-phenylpropanoic acid (107.52 mg, 405.27 μmol) was added into the reaction. The reaction mixture was cooled and stirred at 0° C. Then more DIPEA (69.84 mg, 540.36 μmol, 94.12 μL) was added in one portion at this temperature. The resulting mixture was stirred at room temperature for 31 h. The solution was purified by C18 flash chromatography column, elution gradient from 0 to 80% MeCN in water (6 mmol/L NH₄HCO₃). Pure fractions were lyophilized to dryness to afford amide (R)-enantiomer C-14c (15 mg, 16% yield) as white solid. LCMS: t_{R}=2.18 min in 3 min chromatography (3 min-5-95% MeCN in water (0.02% TFA), Waters Acquity UPLC BEH C18 1.7 μm, 2.1*50 mm, 40° C.), MS (ESI) m/z=700.5 [M+H]⁺.

Step 3: To a solution of (R)-enantiomer C-14c (15 mg, 21.43 μmol) in DCM (1.00 mL) was cooled at 0° C. in ice/water bath, and stirred. Then added 4 M HCl (72.76 mg, 2.00 mmol, 0.50 mL) dropwise into the reaction. The resulting mixture was stirred at 0° C. for 1 hr. The reaction mixture was concentrated under reduced pressure. The residue was purified by C18-flash chromatography, elution gradient from 0% to 50% MeCN in water (6 mmol/L NH₄HCO₃). Pure fractions were lyophilized to dryness to afford (R)-enantiomer C-14d (10 mg, 93% yield) as white solid. LCMS: t_{R}=1.59 min in 3 min chromatography (3 min-5-95% MeCN in water (0.02% TFA), Waters Acquity UPLC BEH C18 1.7 μm, 2.1*50 mm, 40° C.), MS (ESI) m/z=500.2 [M+H]⁺.

Step 4: A solution of (R)-enantiomer C-14d (10 mg, 20.02 μmol) was dissolved into 1 M TBAF in THF (104.67 mg, 400.34 μmol, 0.40 mL). The reaction was sealed and heated at 80° C. for 16 h. The reaction solution was evaporated under reduced pressure. The residue was purified by C18-flash chromatography column, elution gradient from 0 to 30% CH₃CN in water (0.02% TFA). Pure fractions were evaporated to dryness to afford (R)-enantiomer Example 14 (4 mg, 8.73 μmol, 44% yield, TFA salt) brown solid. LCMS: t_{R}=1.23 min in 3 min chromatography (3 min-5-95% MeCN in water (0.02% TFA), Waters Acquity UPLC BEH C18 1.7 μm, 2.1*50 mm, 40° C.), MS (ESI) m/z=346.3 [M+H]⁺. ¹H NMR: (400 MHZ, DMSO-d₆) δ 10.75 (s, 1H), 10.20 (s, 1H), 7.97 (d, J=12.6 Hz, 5H), 7.66-7.56 (m, 2H), 7.48 (d, J=7.4 Hz, 2H), 7.42 (t, J=7.4 Hz, 2H), 7.35 (d, J=7.5 Hz, 2H), 6.99 (t, J=7.9 Hz, 1H), 4.21 (dd, J=9.4, 4.6 Hz, 1H), 3.58-3.55 (m, 1H), 3.16-3.12 (m, 1H).

### Example 15

### Preparation of(S)—N-(3-(1H-pyrazol-4-yl)-1H-indol-7-yl)-3-amino-2-phenylpropanamide

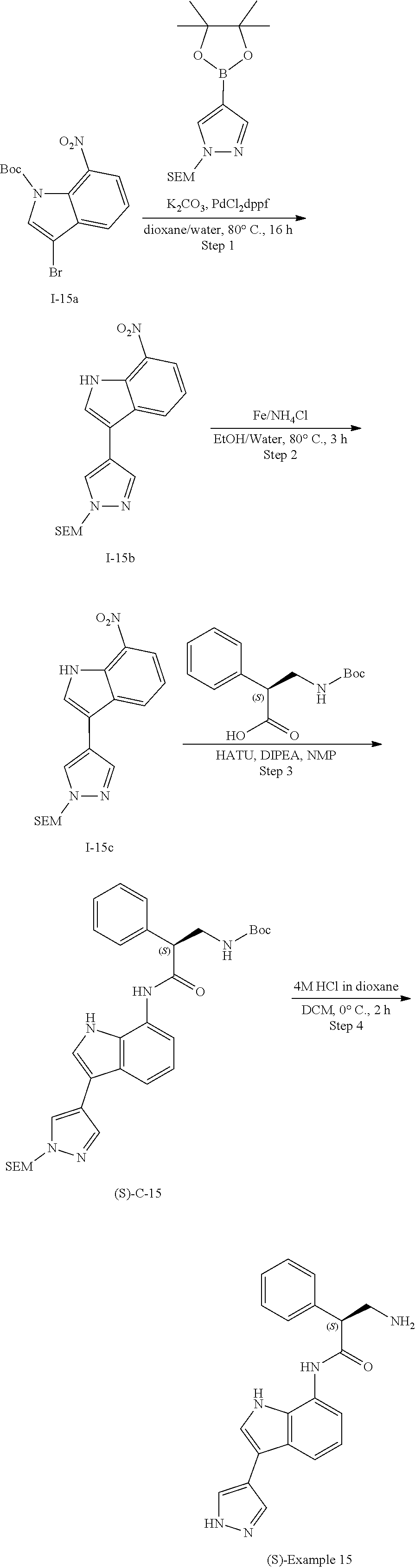

Step 1: nitro-indole I-15a (219 mg, 641.93 μmol), 4-(4,4,5,5-tetramethyl-1,3,2-dioxaborolan-2-yl)-1-((2-(trimethylsilyl) ethoxy)methyl)-1H-pyrazole (249.81 mg, 770.32 μmol), potassium carbonate (266.16 mg, 1.93 mmol, 116.23 μL) and PdCl₂dppf (52.42 mg, 64.19 μmol) in dioxane (5.00 mL) and water (1 mL) were stirred under nitrogen at 90° C. for 16 h. The solvent was removed under reduced pressure. The residue was purified by silica column chromatography, elution gradient from 0 to 10% methanol in DCM. Pure fractions were evaporated to dryness to afford pyrazole I-15b (200 mg, 87% yield) as brown solid. LCMS: t_{R}=2.01 min in 3 min chromatography (3 min-5-95% MeCN in water (0.02% TFA), Waters Acquity UPLC BEH C18 1.7 μm, 2.1*50 mm, 40° C.), MS (ESI) m/z=359.3 [M+H]⁺.

Step 2: To a mixture of pyrazole I-15b (200 mg, 557.93 μmol) and ammonia hydrochloride (298.45 mg, 5.58 mmol) in ethanol (10 mL) and water (10 mL) was added iron powder (155.79 mg, 2.79 mmol). Then the reaction mixture was stirred and heated at 80° C. for 3 h. The reaction mixture was filtered through celite and washed with ethanol (10 mL×3), and the filtrate was concentrated. The residue was dissolved 50 mL of ethyl acetate, filtered and the filtration was concentrated under reduced pressure to afford amine I-15c (160 mg, 87% yield) as brown solid. LCMS: t_{R}=1.56 min in 3 min chromatography (3 min-5-95% MeCN in water (0.02% TFA), Waters Acquity UPLC BEH C18 1.7 μm, 2.1*50 mm, 40° C.), MS (ESI) m/z=329.3 [M+H]⁺.

Step 3: To a stirred mixture of(S)-3-((tert-butoxycarbonyl)amino)-2-phenylpropanoic acid (40.38 mg, 152.21 μmol), amine I-15c (50 mg, 152.21 μmol) and DIPEA (59.02 mg, 456.64 μmol, 79.54 μL) in NMP (2 mL) was cooled and stirred at 0° C. Then HATU (69.82 mg, 183.63 μmol) was added in one portion at this temperature. The resulting mixture was stirred at 0° C. for 2 h. The solution was purified by C18 flash chloramatography column, elution gradient from 0 to 60% MeCN in water. Pure fractions were lyophilized to dryness to afford amide(S)-enantiomer C-15 (38 mg, 43% yield) as white solid. LCMS: t_{R}=2.08 min in 3 min chromatography (3 min-5-95% MeCN in water (0.02% TFA), Waters Acquity UPLC BEH C18 1.7 μm, 2.1*50 mm, 40° C.), MS (ESI) m/z=576.6 [M+H]⁺.

Step 4: To a solution of amide(S)-enantiomer C-15 (38 mg, 66.00 μmol) in DCM (2.00 mL) was cooled at 0° C. in ice/water bath, and stirred. Then added 4 M HCl (145.83 mg, 4.00 mmol, 1.00 mL) dropwise into the reaction. The resulting mixture was stirred at 0° C. for 2 h. The reaction mixture was concentrated under reduced pressure. The residue was purified by C18-flash chromatography, elution gradient from 0% to 50% MeCN in water (6 mmol/L NH₄HCO₃). Pure fractions were lyophilized to dryness to afford(S)-enantiomer Example 15 (3.2 mg, 14% yield) as white solid. LCMS: t_{R}=1.17 min in 3 min chromatography (3 min-5-95% MeCN in water (0.02% TFA), Waters Acquity UPLC BEH C18 1.7 μm, 2.1*50 mm, 40° C.), MS (ESI) m/z=346.2 [M+H]⁺. ¹H NMR: (400 MHZ, DMSO-d₆) δ 10.87 (s, 1H), 7.95 (s, 2H), 7.58 (d, J=8.1 Hz, 2H), 7.43 (t, J=8.8 Hz, 3H), 7.40-7.32 (m, 3H), 7.29 (t, J=7.1 Hz, 1H), 6.98 (t, J=7.7 Hz, 1H), 4.07-3.97 (m, 1H), 3.61-3.56 (m, 1H), 3.00-2.93 (m, 1H).

### Example 16 (Racemic), Example 17 (Enantiomer 1), and Example 18 (Enantiomer 2)

### Preparation of N-(3-(1H-pyrazol-4-yl)-1H-indol-7-yl)-3-amino-2-(3-methoxyphenyl)propanamide

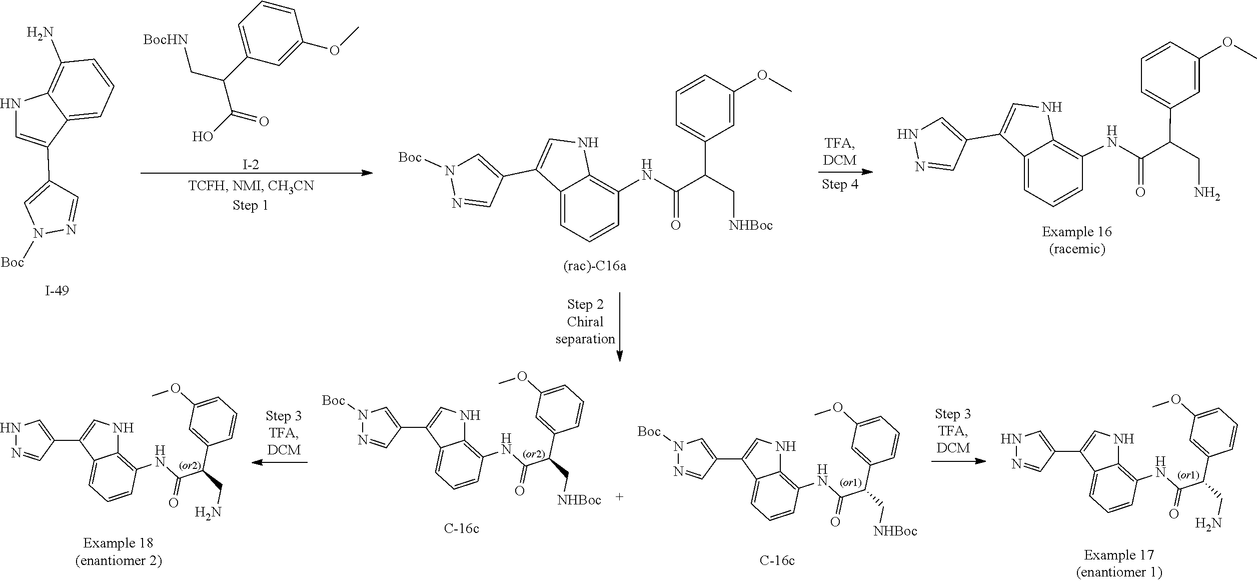

Step 1: Into a 20 mL vial were added acid I-2 (237.58 mg, 0.80 mmol, 1.2 equiv), TCFH (282.14 mg, 1.01 mmol, 1.5 equiv), NMI (220.16 mg, 2.68 mmol, 4 equiv) and amine I-49 (200 mg, 0.67 mmol, 1.00 equiv) in CH₃CN (1.5 mL) at room temperature. The resulting mixture was stirred for 2 h at room temperature under nitrogen atmosphere. The resulting mixture was concentrated under reduced pressure. The residue was purified by prep-TLC with DCM/methanol (30:1, v/v) to afford amide C-16a (150 mg, 38.87%) as a yellow solid. LCMS: m/z (ESI), [M+H]⁺=576.30.

Step 2: The racemic amide C-16a was purified by prep-chiral HPLC with the following conditions (Column: CHIRALPAK IG, 2*25 cm, 5 um; Mobile Phase A: Hex (0.2% DEA), Mobile Phase B: EtOH:DCM=1:1, v/v; Flow rate: 20 mL/min; Gradient: 25% B in 23 min; wavelength: 220/254 nm; t_{R}-1=14.66 min; t_{R}-2=19.11 min; Sample Solvent: EtOH:DCM=1:1, v/v; Injection Volume: 0.3 mL) to afford enantiomer C-16b and enantiomer C-16c as white solids.

Step 3: Into a 50 mL round-bottom flask were added enantiomer C-16c (50 mg, 0.087 mmol, 1 equiv) and DCM (2 mL) in TFA (1 mL) at room temperature. The resulting mixture was stirred for 1.5 h at room temperature. The mixture was basified to pH 9 with saturated NaHCO₃ (aq). The resulting mixture was extracted with CH₂Cl₂ (3×10 mL). The combined organic layers were concentrated under reduced pressure. The crude product was purified by prep-HPLC with the following conditions (Column: XBridge Prep OBD 30*150 mm, 5 um; Mobile Phase A: water (10 mmol/L NH₄HCO₃), Mobile Phase B: ACN; Flow rate: 60 mL/min; Gradient: 10% B to 40% B in 7 min; wavelength: 220 nm; t_{R}=6.25 min) to afford Example 18, enantiomer 2 (14.0 mg, 42.93%) as a white solid. LCMS: m/z (ESI), [M+H]⁺=376.15. ¹H NMR (CD₃OD, 400 MHZ) δ 3.05 (1H, dd), 3.43 (1H, dd), 3.83 (3H, s), 3.93 (1H, dd), 6.90 (1H, ddd), 7.00-7.13 (4H, m), 7.26-7.35 (1H, m), 7.47 (1H, s), 7.64 (1H, dd), 7.91 (2H, s). SFC: t_{R}=3.248, ee=97.14%.

Into a 50 mL round-bottom flask were added enantiomer C-16b (50 mg, 0.087 mmol, 1 equiv) and DCM (2 mL) in TFA (1 mL) at room temperature. The resulting mixture was stirred for 1.5 h at room temperature. The mixture was basified to pH 7 with saturated NaHCO₃ (aq). The resulting mixture was extracted with CH₂Cl₂ (3×10 mL). The combined organic layers were concentrated under reduced pressure. The crude product was purified by prep-HPLC with the following conditions (Column: XBridge Shield RP18 OBD Column, 30*150 mm, 5 um; Mobile Phase A: water (10 mmol/L NH₄HCO₃+0.1% NH₃·H₂O), Mobile Phase B: ACN; Flow rate: 60 mL/min; Gradient: 18% B to 40% B in 8 min; wavelength: 220 nm; t_{R}=7.52 min) to afford Example 17, enantiomer 1 (4.3 mg, 13.19%) as a white solid. LCMS: m/z (ESI), [M+H]⁺=376.15. ¹H NMR (CD₃OD, 400 MHz) δ 3.05 (1H, dd), 3.43 (1H, dd), 3.83 (3H, s), 3.93 (1H, dd), 6.90 (1H, ddd), 7.00-7.13 (4H, m), 7.26-7.35 (1H, m), 7.47 (1H, s), 7.64 (1H, dd), 7.91 (2H, s). SFC: t_{R}=3.744 min, ee=95.56%.

Step 4: Into a 25 mL round-bottom flask were added racemic amide C-16a (20 mg, 0.035 mmol, 1 equiv) and TFA (1 mL) in DCM (2 mL) at room temperature. The resulting mixture was stirred for 2 h at room temperature. The mixture was basified to pH 9 with saturated NaHCO₃. The resulting mixture was extracted with CH₂Cl₂ (3×50 mL). The crude product was purified by prep-HPLC with the following conditions (Column: XBridge Prep OBD C18 30*150 mm, 5 um; Mobile Phase A: water (10 mmol/L NH₄HCO₃), Mobile Phase B: ACN; Flow rate: 60 mL/min; Gradient: 14% B to 39% B in 8 min; wavelength: 220 nm; t_{R}=6.97 min) to afford amide Example 16, racemic (1.6 mg, 12.27%) as a white solid. LCMS: m/z (ESI), [M+H]⁺=376.20. ¹H NMR (CD₃OD, 400 MHZ) δ 3.04-3.08 (1H, m), 3.32-3.50 (1H, m), 3.83 (3H, s), 3.93 (2H, dd), 6.90 (1H, ddd), 6.99-7.18 (4H, m), 7.32 (1H, t), 7.47 (1H, s), 7.64 (1H, dd), 7.92 (2H, s).

### Example 19 (Racemic), Example 20 (Enantiomer 1), and Example 21 (Enantiomer 2)

### Preparation of N-(3-(1H-pyrazol-4-yl)-1H-indol-7-yl)-3-amino-2-(3-cyanophenyl)propanamide

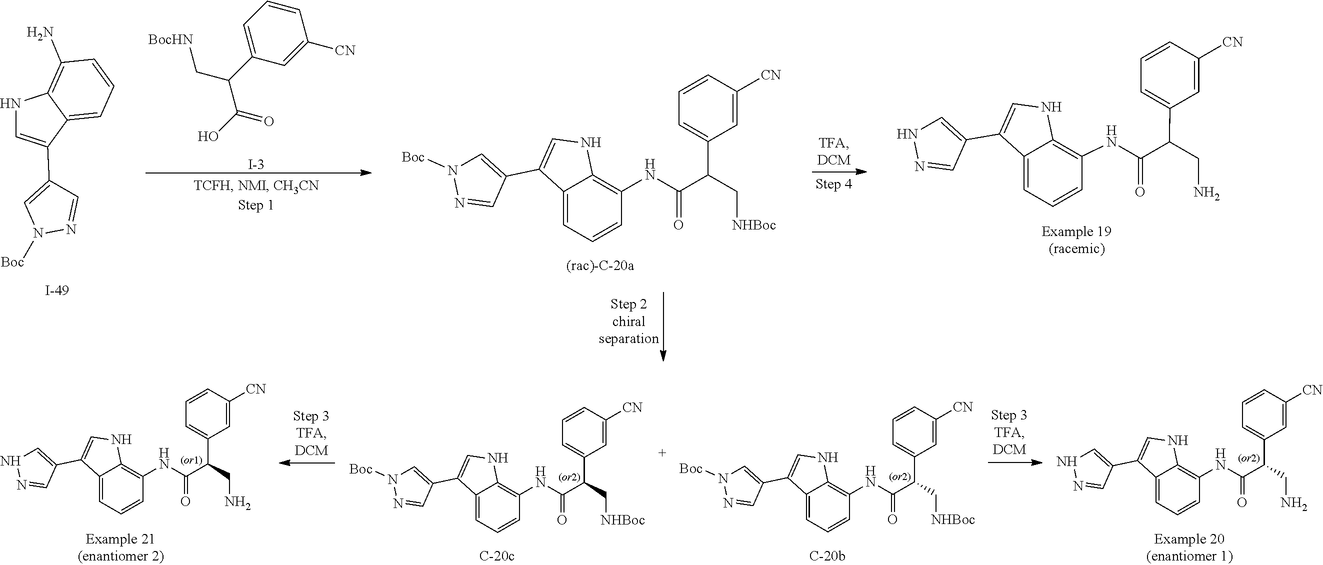

Step 1: To a solution of acid I-3 (379.51 mg, 1.31 mmol, 1.2 equiv) and amine I-49 (325 mg, 1.09 mmol, 1 equiv) in CH₃CN (20 mL) were added TCFH (366.78 mg, 1.31 mmol, 1.2 equiv) and NMI (357.77 mg, 4.36 mmol, 4 equiv) at 25° C. under N₂ atmosphere. The mixture was stirred for 2 hours at 25° C. The resulting mixture was filtered and the filtered cake was washed with methanol (3×100 mL). The filtrate was concentrated under reduced pressure. The residue was purified by prep-TLC with PE/EA (1:1, v/v) to afford racemic amide C-20a (350 mg, 54.46%) as a brown solid. LCMS: m/z (ESI), [M+H]⁺=571.30.

Step 2: The racemic amide C-20a (350 mg, 0.61 mmol, 1 equiv) was purified by Chiral-HPLC with the following conditions (Column: CHIRALPAK IG, 2*25 cm, 5 um; Mobile Phase A: Hex (0.5% 2M NH₃-methanol), Mobile Phase B: EtOH:DCM (1:1, v/v); Flow rate: 20 mL/min; Gradient: 40% B to 40% B in 8.5 min; wavelength: 220/254 nm; t_{R}-1=5.60 min; t_{R}-2=7.14 min. Sample Solvent: EtOH:DCM (1:1, v/v); Injection Volume: 0.6 mL) to afford enantiomer amide C-20c (112 mg, 31.68%) as a white solid. LCMS m/z (ESI), [M+H]⁺=571.30. Chiral HPLC: t_{R}=1.336 min, ee=100% and enantiomer amide C-20b (119 mg, 33.66%) as a white solid. LCMS: m/z (ESI), [M+H]⁺=571.30. Chiral HPLC: t_{R}=1.735 min, ee=100%.

Step 3: To a solution of enantiomer amide C-20c (102 mg, 0.18 mmol, 1 equiv) in DCM (15 mL) was added TFA (5 mL) at 25° C. under N₂ atmosphere. The mixture was stirred for 2 hours at 25° C. The resulting mixture was concentrated under reduced pressure. The residue was dissolved in methanol (50 mL). The mixture was basified to pH 8 with NH₃ (aq). The resulting mixture was concentrated under vacuum. The crude product was purified by prep-HPLC with the following conditions (Column: XBridge Shield RP18 OBD Column, 30*150 mm, 5 um; Mobile Phase A: water 10 mmol/L NH₄HCO₃), Mobile Phase B: ACN; Flow rate: 60 mL/min; Gradient: 13% B to 39% B in 8 min; wavelength: 220 nm; t_{R}=7.58 min) to afford Example 21, enantiomer 2 (51.4 mg, 76.95%) as a white solid. LCMS m/z (ESI), [M+H]⁺=371.10. ¹H NMR (CD₃OD, 400 MHZ) δ 3.03 (1H, m), 3.42 (1H, m), 3.98 (1H, dt), 7.06 (2H, m), 7.47 (1H, s), 7.58 (1H, m), 7.67 (2H, m), 7.80 (1H, m), 7.86 (1H, t), 7.91 (2H, s). SFC: t_{R}=2.432 min, ee=98.42%.

To a solution of enantiomer amide C-20b (109 mg, 0.19 mmol, 1 equiv) in DCM (15 mL) was added TFA (5 mL) at 25° C. under N₂ atmosphere. The mixture was stirred for 2 hours at 25° C. The resulting mixture was concentrated under reduced pressure. The residue was dissolved in methanol (50 mL). The mixture was basified to pH 8 with NH₃ (aq). The resulting mixture was concentrated under vacuum. The crude product was purified by prep-HPLC with the following conditions (Column: XBridge Shield RP18 OBD Column, 30*150 mm, 5 um; Mobile Phase A: water (10 mmol/L NH₄HCO₃), Mobile Phase B: ACN; Flow rate: 60 mL/min; Gradient: 13% B to 39% B in 8 min; wavelength: 220 nm; t_{R}=7.57 min) to afford Example 20, enantiomer 1 (37.4 mg, 52.10%) as a white solid. LCMS m/z (ESI), [M+H]⁺=371.10. ¹H NMR (CD₃OD, 400 MHZ) δ 3.04 (1H, dd), 3.43 (1H, dd), 3.98 (1H, dd), 7.06 (2H, m), 7.47 (1H, s), 7.58 (1H, t), 7.68 (2H, m), 7.81 (1H, dt), 7.87 (1H, d), 7.92 (2H, s). SFC: t_{R}=1.999 min, ee=100%.

Step 4: A mixture of TFA (2 mL) and racemic amide C-20a (170 mg, 0.30 mmol, 1 equiv) in DCM (6 mL) was stirred for 1 h at room temperature under air atmosphere. The resulting mixture was concentrated under reduced pressure. The resulting mixture was diluted with water (20 mL). The mixture was basified to pH 8 with saturated NaHCO₃ (aq.).

The resulting mixture was extracted with ethyl acetate (3×20 mL), dried over anhydrous Na₂SO₄. After filtration, the filtrate was concentrated under reduced pressure. The crude product (100 mg) was purified by prep-HPLC with the following conditions to afford racemic Example 19 (30 mg, 26.68%) as a white solid. LCMS: m/z (ESI), [M+H]⁺=371.10. ¹H NMR (400 MHZ, DMSO-d₆) δ 3.06 (1H, d), 3.45 (1H, d), 4.01 (1H, d), 7.02-7.10 (2H, m), 7.47 (1H, s), 7.59 (1H, t), 7.62-7.68 (1H, m), 7.71 (1H, t), 7.82 (1H, t), 7.86-7.94 (3H, m).

### Example 22 (Racemic)

### Preparation of 3-amino-2-cyclohexyl-N-[3-(1H-pyrazol-4-yl)-1H-indol-7-yl]propanamide (racemic)

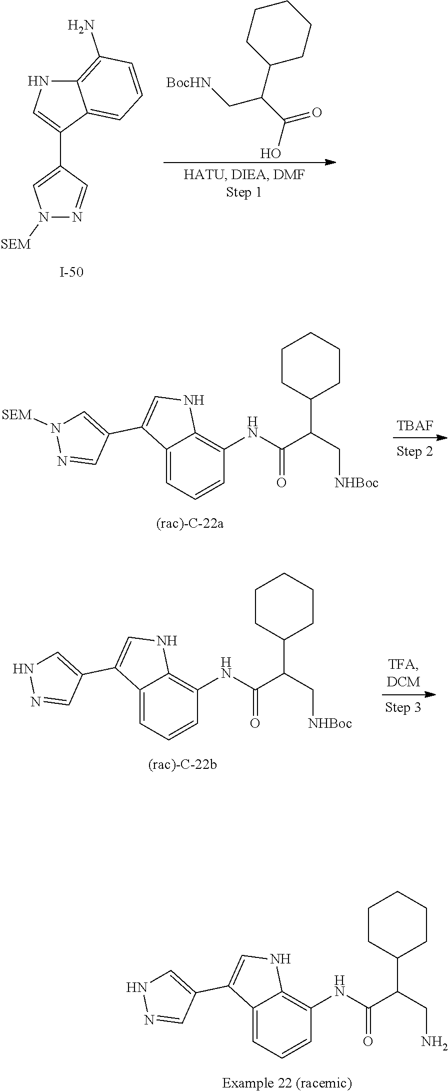

Step 1: To a stirred solution of amine I-50 (110 mg, 0.34 mmol, 1 equiv) and 3-[(tert-butoxycarbonyl)amino]-2-cyclohexylpropanoic acid (99.95 mg, 0.37 mmol, 1.1 equiv) in DMF (5 mL) was added HATU (190.99 mg, 0.50 mmol, 1.5 equiv) and DIEA (129.84 mg, 1.01 mmol, 3 equiv) in portions at room temperature under nitrogen atmosphere. The resulting mixture was stirred for 2 h at room temperature under nitrogen atmosphere. The crude product was purified by reverse flash chromatography with the following conditions: column, C18 silica; mobile phase, MeCN in water, 0% to 100% gradient in 20 min; detector, UV 254 nm. The resulting mixture was concentrated under vacuum. This resulted racemic amide C-22a (70 mg, 35.93%) as a white solid. LCMS: m/z (ESI), [M+H]⁺=582.50.

Step 2: A solution of racemic amide C-22a (40 mg, 0.069 mmol, 1 equiv) in TBAF (7M) in THF (2 mL) was stirred for overnight at room temperature under nitrogen atmosphere. The resulting mixture was diluted with water (10 mL). The resulting mixture was extracted with DCM (3×20 mL). The combined organic layers dried over anhydrous Na₂SO₄. After filtration, the filtrate was concentrated under reduced pressure. The residue was purified by prep-TLC with DCM/methanol (15:1, v/v) to afford pyrazole C-22b (15 mg, 48.32%) as a white solid. LCMS: m/z (ESI), [M+H]⁺=452.30.

Step 3: A solution of pyrazole C-22b (15 mg, 0.033 mmol, 1 equiv) and TFA (0.5 mL) in DCM (1 mL) was stirred for 2 h at room temperature under nitrogen atmosphere. The crude product was purified by prep-HPLC with the following conditions (Column: XBridge Prep OBD 30*150 mm, 5 um; Mobile Phase A: water (10 mmol/L NH₄HCO₃), Mobile Phase B: ACN; Flow rate: 60 mL/min; Gradient: 17% B to 42% B in 8 min; Wavelength: 254 nm; t_{R}=7.47 min) to afford racemic Example 22 (2 mg, 16.57%) as a white solid. LCMS: m/z (ESI), [M+H]⁺=352.20. ¹H NMR (400 MHZ, DMSO-d₆) δ 1.06-0.95 (1H, m), 1.31-1.10 (4H, m), 12.81 (1H, s), 1.91-1.50 (6H, m), 2.38 (1H, td), 2.96-2.81 (2H, m), 7.00 (1H, t), 7.43 (1H, d), 7.62-7.53 (2H, m), 7.95 (2H, s), 9.76 (1H, s), 10.92 (1H, s).

### Example 23 (Enantiomer 1) and Example 24 (Enantiomer 2)

### Preparation of N-(3-(1H-pyrazol-4-yl)-1H-indol-7-yl)-3-amino-2-cyclohexylpropanamide

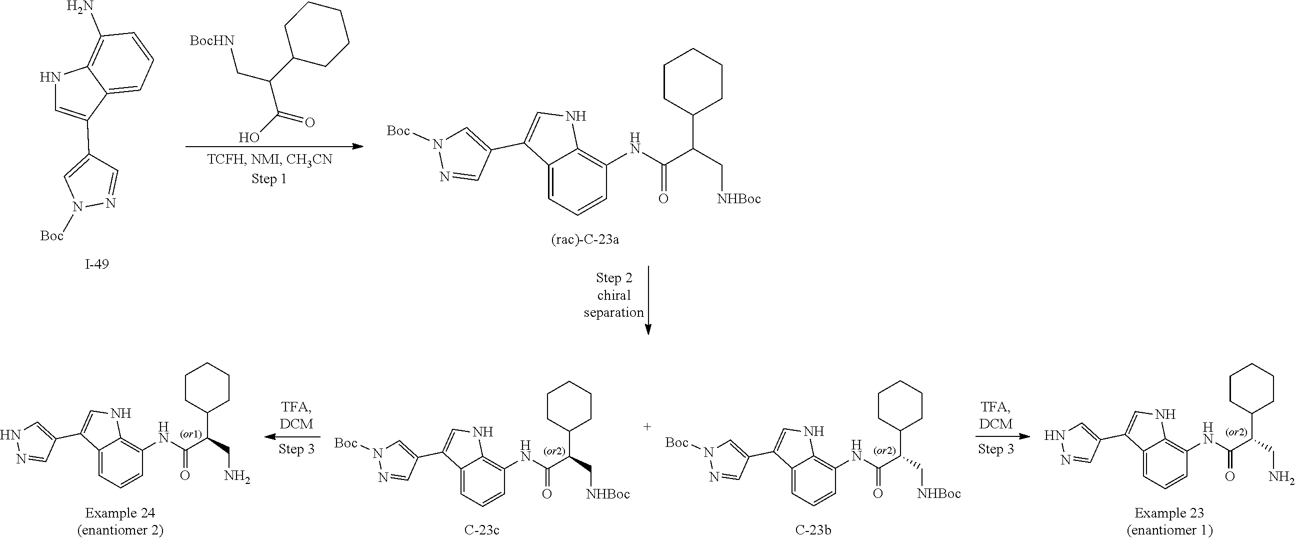

Step 1: Into a 40 mL vial were added amine I-49 (300 mg, 1.01 mmol, 1 equiv) and 3-[(tert-butoxycarbonyl)amino]-2-cyclohexylpropanoic acid (300.15 mg, 1.11 mmol, 1.1 equiv) and TCFH (338.56 mg, 1.21 mmol, 1.2 equiv) and NMI (330.24 mg, 4.02 mmol, 4 equiv) and MeCN (5 mL) at room temperature. The resulting mixture was stirred for 1.5 h at room temperature under nitrogen atmosphere. The reaction was monitored by LCMS. Desired product could be detected by LCMS. The resulting mixture was concentrated under reduced pressure. The residue was purified by prep-TLC (CH₂Cl₂/methanol 45:1) to afford amide C-23a (420 mg, 75.71%) as a yellow solid. LCMS: m/z (ESI), [M+H]⁺=552.40.

Step 2: The amide C-23a (420 mg) was purified by prep-chiral HPLC with the following conditions (Column: CHIRALPAK IE, 2*25 cm, 5 um; Mobile Phase A: MTBE (0.1% DEA), Mobile Phase B: ACN (0.1% IPA); Flow rate: 20 mL/min; Gradient: 10% B to 10% B in 16 min; wavelength: 220/254 nm; t_{R}-1=8.86 min; t_{R}-2=12.61 min; Sample Solvent: IPA; Injection Volume: 0.6 mL) to afford enantiomer C-23c and enantiomer C-23b (170 mg, 40.5%) as a yellow solid. LCMS: m/z (ESI), [M+H]⁺=552.40.

Step 3: Into a 100 mL round-bottom flask were added amide C-23c (200 mg, 0.36 mmol, 1 equiv) and TFA (1 mL) and DCM (3 mL) at room temperature. The resulting mixture was stirred for 1 h at room temperature under air atmosphere. The resulting mixture was concentrated under reduced pressure. The resulting mixture was diluted with DMF (2 mL). The mixture was basified to pH 9 with NH₃ (aq). The resulting mixture was stirred for 30 min at 0° C. under air atmosphere. The crude product (180 mg) was purified by prep-HPLC with the following conditions (Column: XBridge Shield RP18 OBD 30*150 mm, 5 um; Mobile Phase A: water (10 mmol/L NH₄HCO₃+0.1% NH₃ aq), Mobile Phase B: ACN; Flow rate: 60 mL/min; Gradient: 20% B to 45% B in 8 min; wavelength: 220 nm; t_{R}=7.67 min) to afford Example 24, enantiomer 2 (69.4 mg, 53.98%) as a white solid. LCMS: m/z (ESI), [M+H]⁺=352.15. ¹H NMR (DMSO-d₆, 400 MHZ) δ 0.98 (1H, q), 1.18 (4H, p), 1.64 (5H, d), 1.83 (1H, d), 2.37 (1H, s), 2.87 (1H, m), 3.22 (1H, s), 7.00 (1H, m), 7.44 (1H, d), 7.57 (2H, m), 7.95 (2H, s). SFC: t_{R}=1.971 min, ee=97.42%.

Into a 100 mL round-bottom flask were added amide C-23b (170 mg, 0.31 mmol, 1 equiv) and TFA (1 mL) and DCM (3 mL) at room temperature. The resulting mixture was stirred for 1 h at room temperature under air atmosphere. The resulting mixture was concentrated under reduced pressure. The resulting mixture was diluted with DMF (2 mL). The mixture was basified to pH 9 with NH₃ (aq). The resulting mixture was stirred for 30 min at 0° C. under air atmosphere. The crude product (150 mg) was purified by prep-HPLC with the following conditions (Column: YMC-Actus Triart C18 ExRS, 30*150 mm, 5 um; Mobile Phase A: water (10 mmol/L NH₄HCO₃), Mobile Phase B: ACN; Flow rate: 60 mL/min; Gradient: 18% B to 43% B in 7 min; wavelength: 220 nm; t_{R}=6.55 min) to afford Example 23, enantiomer 1 (57 mg, 52.26%) as a white solid. LCMS: m/z (ESI), [M+H]⁺=352.20. ¹H NMR (DMSO-d₆, 400 MHz) δ 1.21 (5H, m), 1.75 (6H, dd), 2.35 (1H, d), 2.86 (1H, m), 3.21 (1H, s), 7.00 (1H, m), 7.44 (1H, m), 7.57 (2H, m), 7.95 (2H, s). SFC: t_{R}=1.860 min, ee=100%.

### Example 26 (Enantiomer 1) and Example 27 (Enantiomer 2)

### Preparation of N-(3-(1H-pyrazol-4-yl)-1H-indol-7-yl)-3-amino-2-(tetrahydro-2H-pyran-4-yl)propanamide

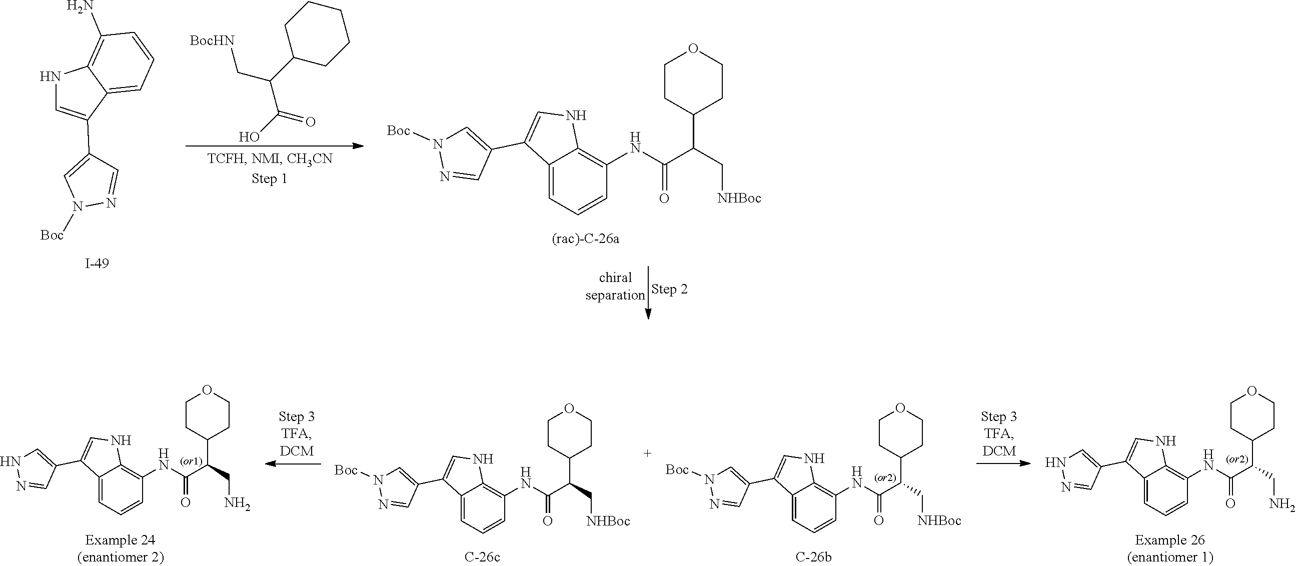

Step 1: Into a 40 mL vial were added 3-[(tert-butoxycarbonyl)amino]-2-(oxan-4-yl)propanoic acid (250 mg, 0.92 mmol, 1 equiv), amine I-49 (272.88 mg, 0.92 mmol, 1 equiv), TCFH (513.26 mg, 1.83 mmol, 2 equiv), NMI (375.49 mg, 4.58 mmol, 5 equiv) and CH₃CN (10 mL) at room temperature. The resulting mixture was stirred for 2 h at room temperature. The residue was purified by prep-TLC (CH₂Cl₂/methanol (25:1, v/v) to afford racemic amide C-26a (312 mg, 59.76%) as a brown solid. LCMS: m/z (ESI), [M−^{t}Bu+H]⁺=498.25.

Step 2: The racemic amide C-26a (312 mg, 59.76%) was purified by prep-chiral HPLC with the following conditions (Column: CHIRALPAK IF, 2*25 cm, 5 um; Mobile Phase A: Hex (0.2% DEA), Mobile Phase B: EtOH:DCM (1:1, v/v); Flow rate: 20 mL/min; Gradient: 30% B to 30% B in 12 min; wavelength: 220/254 nm; t_{R}-1=7.46 min; t_{R}-2=9.02 min; Sample Solvent: EtOH:DCM (1:1, v/v); Injection Volume: 0.2 mL) to afford enantiomer C-26c (130 mg, 41.6%), SFC: t_{R}=1.932 min, ee=100%, and enantiomer C-26b (120 mg, 38.4%) as a white solid, SFC: t_{R}=2.524 min, ee=99.634%.

Step 3: Into a 50 mL round-bottom flask were added enantiomer C-26c (120 mg, 0.22 mmol, 1 equiv) and DCM (3 mL) at room temperature. The resulting mixture was stirred for 1 h at room temperature. The mixture was basified to pH 9 with NH₃ (aq) and stirred for 1 h at 0° C. The crude product (100 mg) was purified by prep-HPLC with the following conditions (Column: XBridge Shield RP18 OBD Column, 30*150 mm, 5 um; Mobile Phase A: water 10 mmol/L NH₄HCO₃+0.1% NH₃ aq), Mobile Phase B: ACN; Flow rate: 60 mL/min; Gradient: 5% B to 30% B in 8 min; wavelength: 220 nm; t_{R}=7.95 min) to afford Example 27, enantiomer 2 (41.3 mg, 53.39%) as a white solid. LCMS: m/z (ESI), [M+H]⁺=354.2. ¹H NMR (CD₃OD, 400 MHZ) δ 1.46 (1H, qd), 1.54-1.73 (2H, m), 1.81 (1H, d), 1.95-1.99 (1H, m), 2.50 (1H, td), 3.00-3.14 (2H, m), 3.44 (2H, dd), 4.00 (2H, s), 7.10 (1H, t), 7.18 (1H, dd), 7.48 (1H, s), 7.67 (1H, dd), 7.93 (2H, s). SFC: t_{R}=2.418 min, ee=99.56%.

Into a 50 mL round-bottom flask were added enantiomer C-26b (120 mg, 0.22 mmol, 1 equiv), TFA (0.02 mL, 0.22 mmol, 1 equiv) and DCM (3 mL) at room temperature. The resulting mixture was stirred for 1 h at room temperature. The mixture was basified to pH 9 with NH₃ (aq). The crude product was purified by prep-HPLC with the following conditions (Column: XBridge Prep OBD 30*150 mm, 5 um; Mobile Phase A: water 10 mmol/L NH₄HCO₃), Mobile Phase B: ACN; Flow rate: 60 mL/min; Gradient: 5% B to 24% B in 9 min; wavelength: 254 nm; t_{R}=8.83 min) to afford Example 26, enantiomer 1 (45.5 mg, 59.24%) as a white solid. LCMS: m/z (ESI), [M+H]⁺=354.2. ¹H NMR (CD₃OD, 400 MHz) δ 1.46 (1H, qd), 1.61 (1H, qd), 1.70 (1H, d), 1.81 (1H, d), 1.90-2.08 (1H, m), 2.51 (1H, td), 3.01-3.15 (2H, m), 3.45 (2H, dt), 3.95-4.05 (2H, m), 7.10 (1H, t), 7.19 (1H, dd), 7.48 (1H, s), 7.67 (1H, dd), 7.92 (2H, s). SFC: t_{R}=2.527 min, ee=98.12%.

### Example 29 (Enantiomer 1) and Example 30 (Enantiomer 2)

### Preparation of N-(3-(1H-pyrazol-4-yl)-1H-indol-7-yl)-2-(3-methoxyphenyl)-2-(1-methylpiperidin-4-yl) acetamide

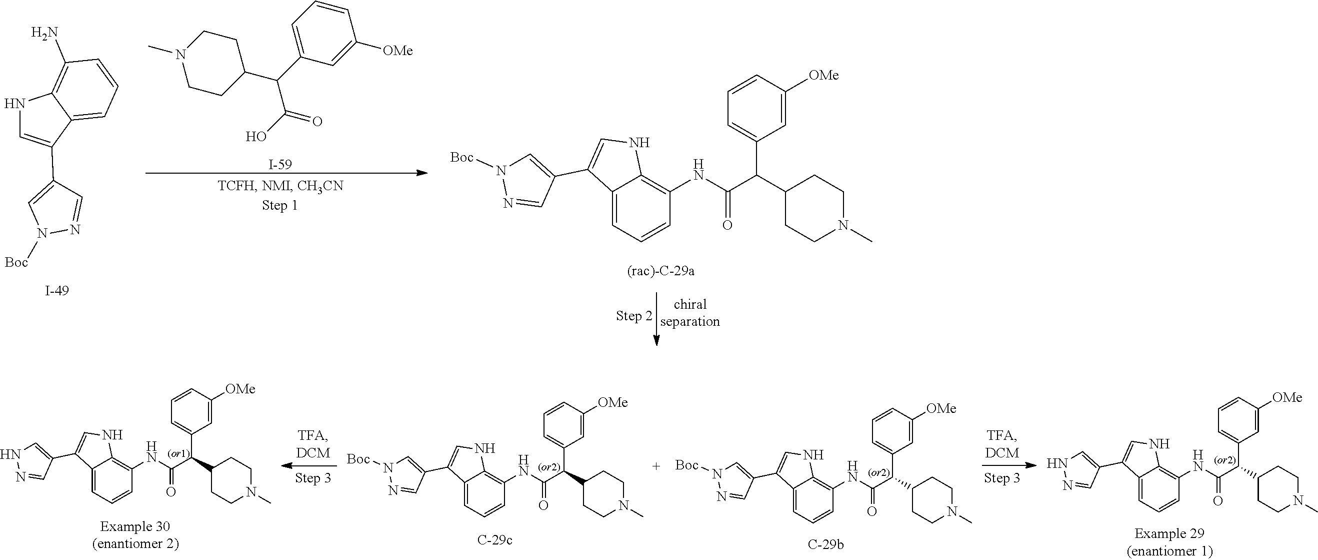

Step 1: Into a 20 mL vial were added acid I-59 (264.80 mg, 1.01 mmol, 1.5 equiv), TCFH (282.14 mg, 1.01 mmol, 1.5 equiv), NMI (220.16 mg, 2.680 mmol, 4.0 equiv) and amine I-49 (200 mg, 0.670 mmol, 1 equiv) in CH₃CN (5 mL) at room temperature. The resulting mixture was stirred for 1.5 h at room temperature under nitrogen atmosphere. The resulting mixture was concentrated under vacuum. The residue was purified by prep-TLC with CH₂Cl₂/methanol (10:1, v/v) to afford racemic amide C-29a (260 mg, 71.34%) as a brown solid. LCMS: m/z (ESI), [M+H]⁺=544.30.

Step 2: The racemic amide C-29a (260 mg) was purified by prep-chiral HPLC with the following conditions (Column: CHIRAL ART Amylose-SA, 2*25 cm, 5 um; Mobile Phase A: Hex (0.2% DEA), Mobile Phase B: EtOH/DCM (1:1, v/v); Flow rate: 20 mL/min; Gradient: 20% B to 20% B in 13 min; wavelength: 220/254 nm; t_{R}-1=5.15 min; t_{R}-2=7.57 min; Sample Solvent: EtOH/DCM (1:1, v/v); Injection Volume: 0.9 mL) to afford enantiomer C-29c, SFC: t_{R}=1.919 min, ee=100%, and enantiomer C-29b as a brown solid, SFC: t_{R}=2.434 min, ee=100%.

Step 3: Into a 50 mL round-bottom flask were added enantiomer C-29c (140 mg, 0.26 mmol, 1 equiv) and TFA (0.5 mL) in DCM (1 mL) at room temperature. The resulting mixture was stirred for 1 h at room temperature. The mixture was basified to pH 9 with NH₃ aq. The crude product was purified by prep-HPLC with the following conditions (Column: XBridge Prep OBD C18 30*150 mm, 5 um; Mobile Phase A: water (10 mmol/L NH₄HCO₃), Mobile Phase B: ACN; Flow rate: 60 mL/min; Gradient: 15% B to 45% B in 7 min; wavelength: 220 nm; t_{R}=7.98 min) to afford Example 30, enantiomer 2 (32.4 mg, 28.37%) as a white solid. LCMS: m/z (ESI), [M+H]⁺=444.20. ¹H NMR (CD₃OD, 400 MHZ) δ 1.18-1.30 (1H, m), 1.37 (1H, dt), 1.54 (1H, qd), 2.02 (2H, ddd), 2.18 (2H, qt), 2.32 (3H, s), 2.87 (1H, d), 2.99 (1H, d), 3.43 (1H, d), 3.83 (3H, s), 6.88 (1H, ddd), 7.01-7.14 (3H, m), 7.20 (1H, dd), 7.29 (1H, t), 7.44 (1H, s), 7.61 (1H, dd), 7.80-7.98 (2H, m). SFC: t_{R}=2.156, ee=100%.

Into a 50 mL round-bottom flask were added enantiomer C-29b (140 mg, 0.26 mmol, 1 equiv) and TFA (0.5 mL, 6.73 mmol, 26.14 equiv) in DCM (1 mL) at room temperature. The resulting mixture was stirred for 1 h at room temperature. The mixture was basified to pH 9 with NH₃ aq. The crude product was purified by prep-HPLC with the following conditions (Column: XBridge Prep OBD C18 30*150 mm, 5 um; Mobile Phase A: water (10 mmol/L NH₄HCO₃), Mobile Phase B: ACN; Flow rate: 60 mL/min; Gradient: 10% B to 45% B in 7 min; wavelength: 220 nm; t_{R}=8.58 min) to afford Example 29, enantiomer 1 (35.2 mg, 23.97%) as a white solid. LCMS: m/z (ESI), [M+H]⁺=444.25. ¹H NMR (CD₃OD, 400 MHZ) δ 1.15-1.29 (1H, m), 1.36 (1H, dt), 1.53 (1H, qd), 1.91-2.06 (2H, m), 2.06-2.26 (2H, m), 2.30 (3H, s), 2.85 (1H, d), 2.97 (1H, d), 3.42 (1H, d), 3.83 (3H, s), 6.88 (1H, ddd), 7.01-7.13 (3H, m), 7.20 (1H, dd), 7.29 (1H, t), 7.45 (1H, s), 7.61 (1H, dd), 7.91 (2H, s). SFC: t_{R}=2.036 min, ee=91.58%.

### Example 31 (Racemic), Example 32 (Enantiomer 1), and Example 33 (Enantiomer 2)

### Preparation of N-(3-(1H-pyrazol-4-yl)-1H-indol-7-yl)-3-amino-2-(pyridin-3-yl)propanamide

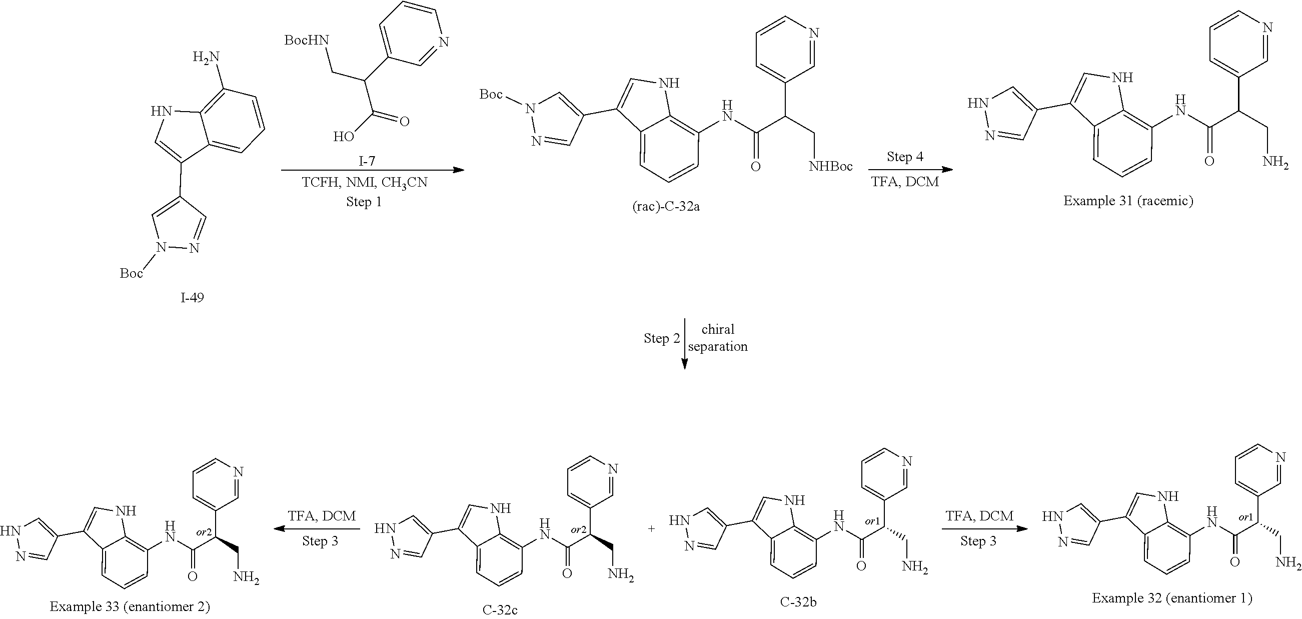

Step 1: To a stirred mixture of acid I-7 (190 mg, 0.71 mmol, 1 equiv) in CH₃CN (3.0 mL) was added NMI (175.75 mg, 2.14 mmol, 3 equiv), TCFH (400.38 mg, 1.43 mmol, 2 equiv) and amine I-49 (217.12 mg, 0.73 mmol, 1.02 equiv) in portion at 25° C.° C. under air atmosphere. The resulting mixture was stirred for overnight at 25° C. under air atmosphere. After reaction, the resulting mixture was diluted with water (10 mL). and extracted with ethyl acetate (3×20 mL). The combined organic layers were washed with water (3×10 mL), dried over anhydrous Na₂SO₄. After filtration, the filtrate was concentrated under reduced pressure. The residue was purified by prep-TLC with CH₂Cl₂/methanol (20:1, v/v) to afford racemic C-32a (220 mg, 56.41%) as a yellow solid. LCMS: m/z (ESI), [M+H]⁺=547.20.

Step 2: The racemic C-32a (220 mg) was purified by prep-HPLC with the following conditions (Column: CHIRALPAK IG, 2*25 cm, 5 um; Mobile Phase A: Hex (0.2% DEA), Mobile Phase B: EtOH:DCM (1:1, v/v); Flow rate: 20 mL/min; Gradient: 35% B to 35% B in 17 min; wavelength: 220/254 nm; t_{R}-1=9.63 min; t_{R}-2=13.49 min; Sample Solvent: EtOH:DCM (1:1, v/v); Injection Volume: 0.5 mL) to afford enantiomer C-32c (80 mg, 36.36%), SFC: t_{R}=2.452 min, ee=100%, and enantiomer C-32b (80 mg, 36.36%) as a white solid, SFC: t_{R}=3.466 min, ee=97.6%.

Step 3: A solution of enantiomer C-32c (80 mg, 0.15 mmol, 1 equiv) in TFA (0.5 mL) and DCM (2.0 mL) was stirred for 0.5 h at room temperature under air atmosphere. After reaction, the resulting mixture was concentrated under vacuum. The residue was dissolved in methanol (1 mL) and basified to pH 9 with NH₃ (aq). The crude product was purified by prep-HPLC with the following conditions (Column: DZ-CHIRALPAK IG-3, 4.6*50 mm, 3.0 um; Mobile Phase: Hex (0.2% DEA):(methanol: DCM (1:1, v/v)=60:40 (v/v); Flow rate: 1 mL/min; Injection Volume: 5 ul mL) to afford Example 33, enantiomer 2 (19 mg, 58.46%) as a white solid. LCMS: m/z (ESI), [M+H]⁺=347.10. ¹H NMR (CD₃OD, 400 MHZ) δ 3.05 (1H, d), 3.45 (1H, t), 3.99 (1H, dd), 7.07 (2H, q), 7.48 (2H, q), 7.65 (1H, dt), 7.92 (2H, s), 8.00 (1H, dt), 8.51 (1H, m), 8.67 (1H, d). Chiral-HPLC: t_{R}=2.308 min, ee=100%.

A solution of enantiomer C-32b (80 mg, 0.15 mmol, 1 equiv) in TFA (0.5 mL) and DCM (2.0 mL) was stirred for 0.5 h at room temperature under air atmosphere. After reaction, the resulting mixture was concentrated under vacuum. The residue was dissolved in methanol (1 mL) and basified to pH 9 with NH₃ (aq). The crude product was purified by prep-HPLC with the following conditions (Column: XBridge Shield RP18 OBD Column, 30*150 mm, 5 um; Mobile Phase A: water (10 mmol/L NH₄HCO₃+0.1% NH₃ aq), Mobile Phase B: ACN; Flow rate: 60 mL/min; Gradient: 8% B to 30% B in 8 min; wavelength: 220 nm; t_{R}=7.87 min) to afford Example 32, enantiomer 1 (20 mg, 39.45%) as a white solid. LCMS: m/z (ESI), [M+H]⁺=347.10. ¹H NMR (CD₃OD, 400 MHz) δ 3.06 (1H, dd), 3.47 (1H, m), 4.00 (1H, dd), 7.07 (2H, m), 7.48 (2H, m), 7.66 (1H, dd), 7.92 (2H, m), 8.01 (1H, d), 8.52 (1H, dd), 8.68 (1H, d). SFC: t_{R}=0.488 min, ee=100%.

Step 4: Into a 50 mL round-bottom flask were added racemic C-32a (60 mg, 0.11 mmol, l equiv), TFA (1 mL) and DCM (3.00 mL) at room temperature. The resulting mixture was stirred for 1 h at room temperature. The resulting mixture was diluted with CH₂Cl₂ (30 mL). The resulting mixture was concentrated under reduced pressure. The resulting mixture was diluted with methanol (2 mL). The mixture was basified to pH 9 with NH₃ (aq). The crude product (50 mg) was purified by prep-HPLC with the following conditions (Column: Xselect CSH F-Phenyl OBD column, 19*250 mm, 5 um; Mobile Phase A: water 10 mmol/L NH₄HCO₃), Mobile Phase B: ACN; Flow rate: 25 mL/min; Gradient: 31% B to 54% B in 8 min; wavelength: 254 nm; t_{R}=7.85 min) to afford Example 1, racemic (11.1 mg, 28.78%) as a white solid. LCMS: m/z (ESI), [M+H]⁺=347.15. ¹H NMR (CD₃OD, 400 MHZ) δ 3.07 (1H, dd), 3.47 (1H, dd), 4.01 (1H, dd), 7.07 (2H, q), 7.44-7.52 (2H, m), 7.65 (1H, dd), 7.92 (2H, s), 8.01 (1H, dt), 8.52 (1H, dd), 8.67 (1H, d).

### Example 35 (Enantiomer 1) and Example 36 (Enantiomer 2)

### Preparation of N-(3-(1H-pyrazol-4-yl)-1H-indol-7-yl)-2-(3-methoxyphenyl)-3-(4-methylpiperazin-1-yl)propanamide

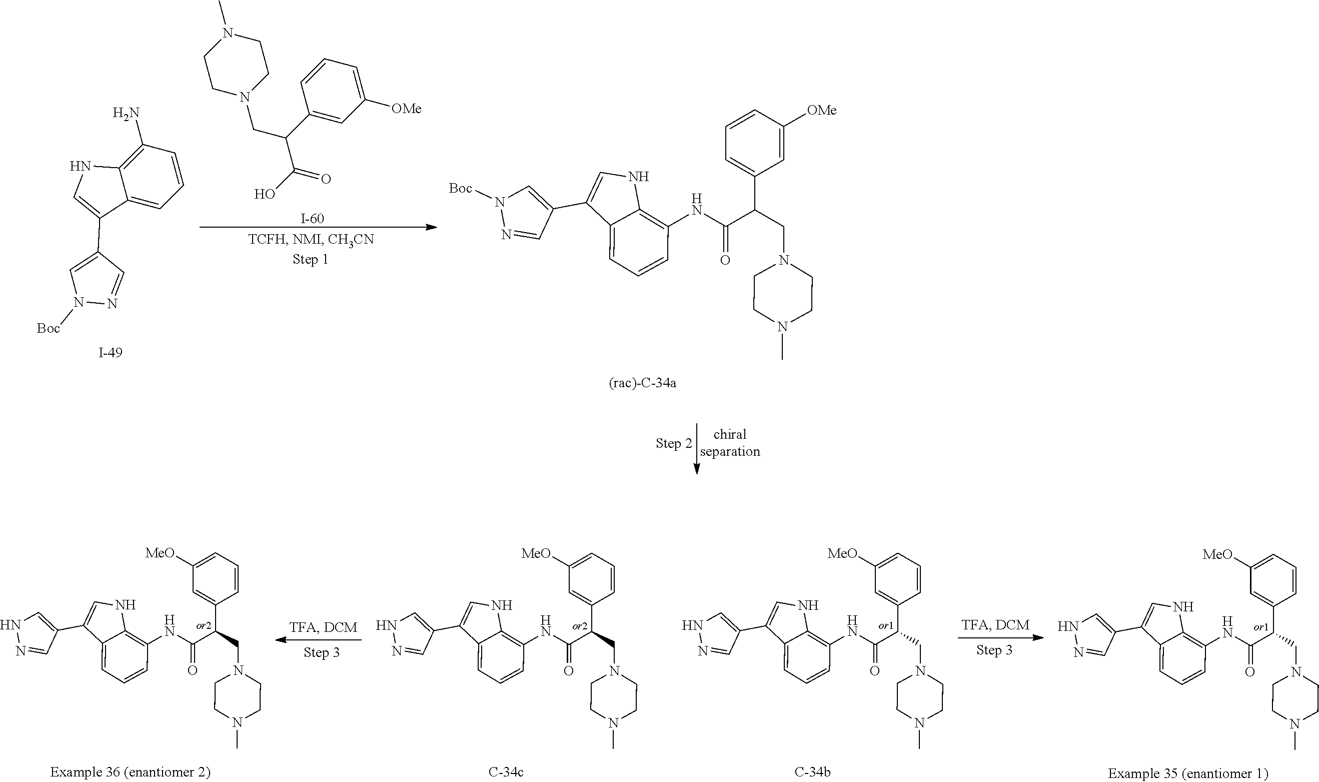

Step 1: Into a 50 mL round-bottom flask were added acid I-60 (233.25 mg, 0.84 mmol, 1 equiv), amine I-49 (250 mg, 0.84 mmol, 1 equiv), TCFH (470.23 mg, 1.676 mmol, 2 equiv), NMI (344.00 mg, 4.19 mmol, 5 equiv) and CH₃CN (10 mL, 190.242 mmol) at room temperature. The resulting mixture was stirred for 1 h at room temperature. The residue was purified by prep-TLC with CH₂Cl₂/MeOH (15:1, v/v) to afford racemic C-34a (600 mg, 115.60%) as a brown solid. LCMS: m/z (ESI), [M+H]⁺=559.35.

Step 2: The racemic C-34a (400 mg) was purified by prep-chiral HPLC with the following conditions (Column: CHIRALPAK IC, 2*25 cm, 5 um; Mobile Phase A: Hex (0.2% DEA), Mobile Phase B: EtOH:DCM (1:1, v/v); Flow rate: 20 mL/min; Gradient: 40% B to 40% B in 10.5 min; wavelength: 220/254 nm; t_{R}-1=6.34 min; t_{R}-1=7.94 min; Sample Solvent: EtOH:DCM (1:1, v/v); Injection Volume: 0.5 mL) to afford enantiomer C-34c (130 mg, 32.5%), SFC: t_{R}=1.787 min, ee=100%, and enantiomer C-34b (150 mg, 37.5%) as a white solid, SFC: t_{R}=2.160 min, ee=95.09%.

Step 3: Into a 50 mL round-bottom flask were added enantiomer C-34c (130 mg, 0.23 mmol, 1 equiv), TFA (1 mL) and DCM (3 mL) at room temperature. The resulting mixture was stirred for 1 h at room temperature. The mixture was basified to pH 9 with NH₃ aq. The crude product (100 mg) was purified by prep-HPLC with the following conditions (Column: XBridge Prep OBD C18 Column, 30*150 mm, 5 um; Mobile Phase A: water (10 mmol/L NH₄HCO₃), Mobile Phase B: ACN; Flow rate: 60 mL/min; Gradient: 15% B to 45% B in 7 min; wavelength: 220 nm; t_{R}=7.83 min) to afford Example 36, enantiomer 2 (66.1 mg, 61.89%) as a white solid. LCMS: m/z (ESI), [M+H]⁺=459.3. ¹H NMR (CD₃OD, 400 MHz) δ 2.31 (3H, d), 2.58 (6H, dd), 2.85 (2H, s), 3.33 (1H, s), 3.44 (1H, t), 3.82 (3H, s), 4.11 (1H, dd), 6.88 (1H, dd), 7.08 (4H, dd), 7.29 (1H, t), 7.50 (1H, s), 7.66-7.69 (1H, m), 7.91 (2H, s). SFC: t_{R}=1.511 min, ee=100%.

Into a 50 mL round-bottom flask were added enantiomer C-34b (160 mg, 0.29 mmol, 1 equiv), TFA (1 mL) and DCM (3 mL) at room temperature. The resulting mixture was stirred for 1 h at room temperature. The mixture was basified to pH 9 with NH₃—H₂O. The crude product (120 mg) was purified by prep-HPLC with the following conditions (Column: XBridge Prep OBD C18 Column, 30*150 mm, 5 um; Mobile Phase A: water (10 mmol/L NH₄HCO₃), Mobile Phase B: ACN; Flow rate: 60 mL/min; Gradient: 15% B to 45% B in 7 min; wavelength: 220 nm; t_{R}=7.78 min) to afford Example 35, enantiomer 1 (29.2 mg, 22.22%) as a white solid. LCMS: m/z (ESI), [M+H]⁺=459.3. ¹H NMR (CD₃OD, 400 MHz) δ 2.31 (3H, d), 2.58 (6H, dd), 2.85 (2H, s), 3.33 (1H, s), 3.44 (1H, t), 3.82 (3H, s), 4.11 (1H, dd), 6.88 (1H, dd), 7.08 (4H, dd), 7.29 (1H, t), 7.50 (1H, s), 7.66-7.69 (1H, m), 7.91 (2H, s). SFC: t_{R}=1.312 min, ee=95.6%.

### Example 37 (Racemic), Example 38 (Enantiomer 1), and Example 39 (Enantiomer 2)

### Preparation of N-(3-(1H-pyrazol-4-yl)-1H-indol-7-yl)-3-(4-methylpiperazin-1-yl)-2-phenylpropanamide

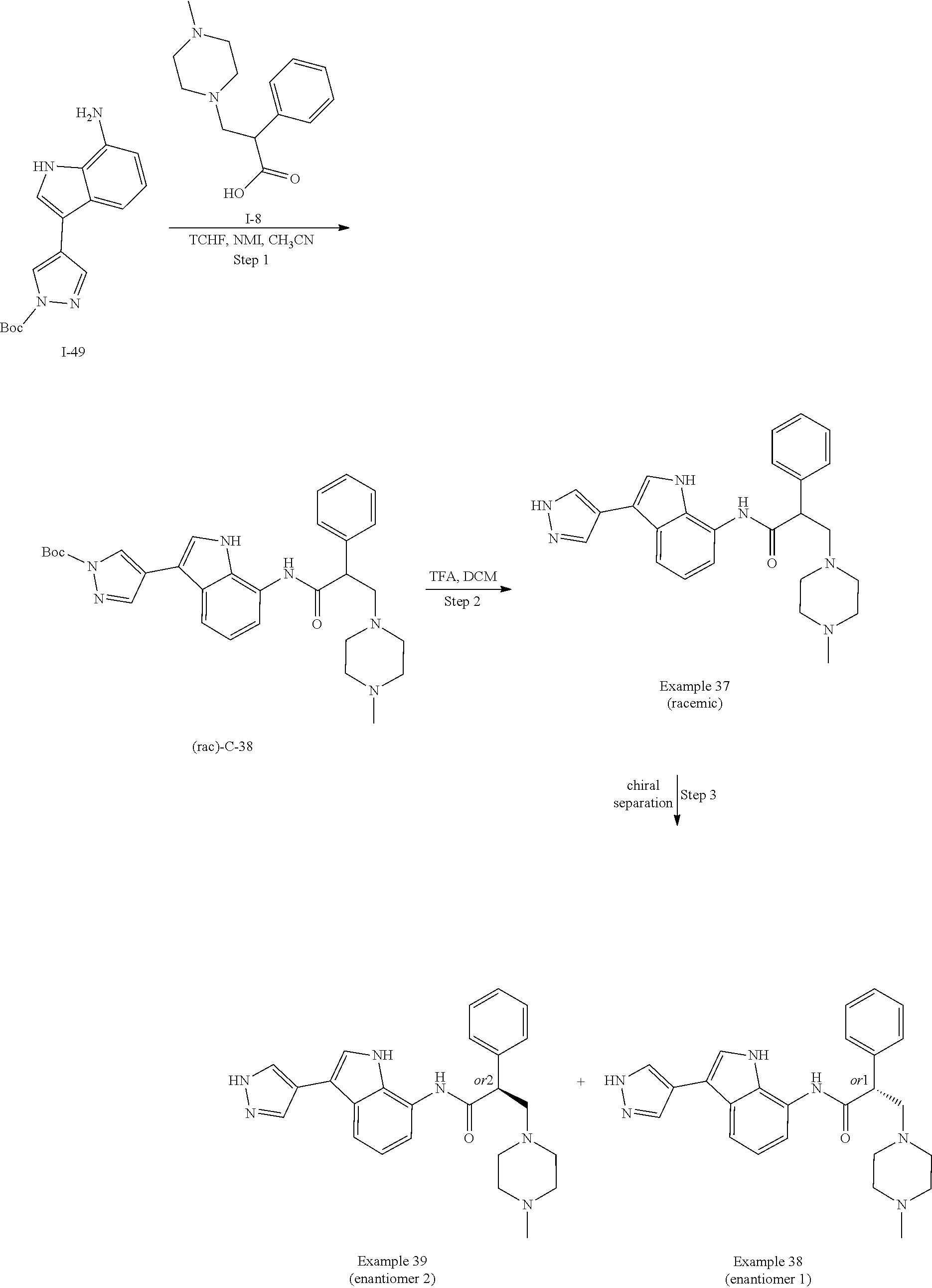

Step 1: To a stirred solution of acid I-8 (200 mg, 0.81 mmol, 1 equiv) and amine I-49 (317.48 mg) in CH₃CN (20 mL) were added TCFH (271.17 mg, 0.97 mmol, 1.2 equiv) and NMI (66.13 mg, 0.81 mmol, 1 equiv) dropwise at room temperature under air atmosphere. The resulting mixture was stirred for 3 h at room temperature under air atmosphere. The resulting mixture was concentrated under reduced pressure. The residue was purified by prep-TLC with CH₂Cl₂/methanol (12:1, v/v) to afford racemic amide C-38 (316 mg, 74.22%) as a brown oil. LCMS: m/z (ESI), [M+H]⁺=529.35.

Step 2: To a stirred solution of racemic amide C-38 (300 mg, 0.568 mmol, 1 equiv) in DCM (25 mL) was added TFA (5 mL) dropwise at room temperature under air atmosphere. The resulting mixture was stirred for 2 h at room temperature under air atmosphere. The reaction was concentrated under reduced pressure. The residue was basified to pH 9 with NH₃ (aq) at 0° C. The crude product was purified by prep-HPLC with the following conditions Column: XBridge Shield RP18 OBD Column, 30*150 mm, 5 um; Mobile Phase A: water (10 mmol/L NH₄HCO₃+0.1% NH₃ aq), Mobile Phase B: ACN; Flow rate: 60 mL/min; Gradient: 22% B to 43% B in 8 min; wavelength: 220 nm; t_{R}=7.80 min) to afford Example 37, racemic (200 mg, 82.24%) as a white solid. LCMS: m/z (ESI), [M+H]⁺=429.25. ¹H NMR (DMSO-d₆, 400 MHz) δ 2.14 (3H, s), 2.34 (4H, s), 2.48 (2H, d), 2.68 (2H, s), 3.22-3.31 (2H, m), 4.11 (1H, dd), 7.01 (1H, t), 7.27 (1H, d), 7.34-7.49 (5H, m), 7.57-7.64 (2H, m), 7.93 (2H, d), 10.03 (1H, s), 10.52 (1H, s), 12.82 (1H, s).

Step 3: The Example 37, racemic (200 mg) was purified by prep-HPLC with the following conditions (Column: DZ-CHIRALPAK IG-3, 4.6*50 mm, 3.0 um; Mobile Phase A: Hex (0.2% DEA): (EtOH:DCM (1:1, v/v))=60:40 (v/v); Flow rate: 1 mL/min; Injection Volume: 5 ul mL) to afford Example 39, enantiomer 2 (58.0 mg, 29.00%). LCMS: m/z (ESI), [M+H]⁺=429.15. ¹H NMR (DMSO-d₆, 400 MHZ) δ 2.14 (3H, s), 2.34 (4H, s), 2.48 (2H, d), 2.68 (2H, s), 3.22-3.31 (2H, m), 4.11 (1H, dd), 7.01 (1H, t), 7.27 (1H, d), 7.34-7.39 (3H, m), 7.41-7.49 (2H, t) 7.57-7.64 (2H, m), 7.93 (2H, d), 10.03 (1H, s), 10.52 (1H, s), 12.82 (1H, s). Chiral-HPLC: t_{R}=1.760 min, ee=100% and Example 38, enantiomer 1 (71.4 mg, 35.70%) as a white solid. LCMS: m/z (ESI), [M+H]⁺=429.15. ¹H NMR (DMSO-d₆, 400 MHz) δ 2.14 (3H, s), 2.34 (4H, s), 2.48 (2H, d), 2.68 (2H, s), 3.22-3.31 (2H, m), 4.11 (1H, dd), 7.01 (1H, t), 7.27 (1H, d), 7.34-7.39 (3H, m), 7.41-7.49 (2H, t) 7.57-7.64 (2H, m), 7.93 (2H, d), 10.03 (1H, s), 10.52 (1H, s), 12.82 (1H, s). Chiral-HPLC: t_{R}=2.520 min, ee=97.39%.

### Example 41

### Preparation of (2S)-2-amino-2-(3-methoxyphenyl)-N-[3-(1H-pyrazol-4-yl)-1H-indol-7-yl]acetamide

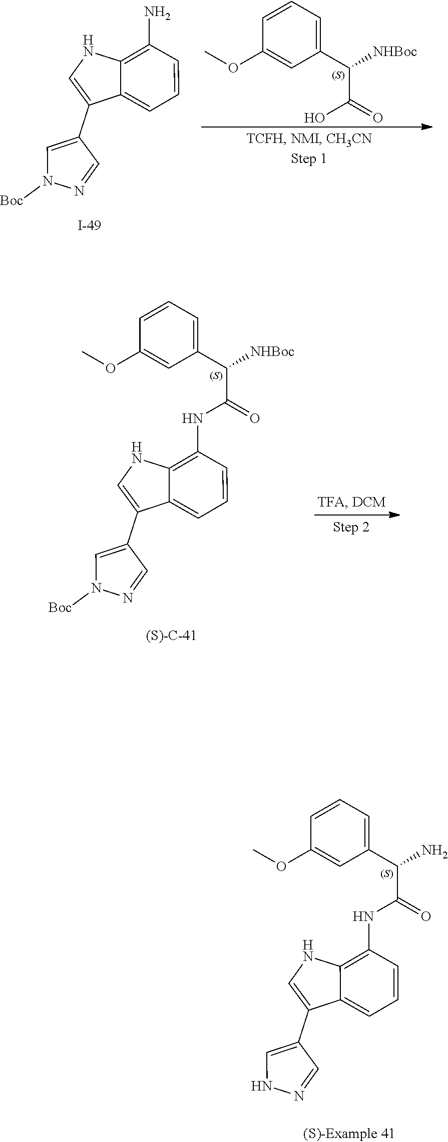

Step 1: Into a 20 mL vial were added amine I-49 (150 mg, 0.50 mmol, 1 equiv) and (S)-[(tert-butoxycarbonyl)amino](3-methoxyphenyl) acetic acid (155.58 mg, 0.55 mmol, 1.1 equiv) and TCFH (169.28 mg, 0.60 mmol, 1.2 equiv) and NMI (165.12 mg, 2.01 mmol, 4 equiv) and CH₃CN (5 mL) at room temperature. The resulting mixture was stirred for 40 min at room temperature under nitrogen atmosphere. The resulting mixture was concentrated under reduced pressure. The residue was purified by prep-TLC with CH₂Cl₂/methanol (25:1, v/v) to afford tert-butyl 4-{7-[(2S)-2-[(tert-butoxycarbonyl)amino]-2-(3-methoxyphenyl) acetamido]-1H-indol-3-yl}pyrazole-1-carboxylate. The crude product (250 mg) was purified by prep-chiral HPLC with the following conditions (Column: CHIRALPAK IE, 2*25 cm, 5 um; Mobile Phase A: Hex (0.2% DEA), Mobile Phase B: EtOH:DCM=1:1 (v/v); Flow rate: 20 mL/min; Gradient: 30% B to 30% B in 18 min; wavelength: 220/254 nm; t_{R}−1=10.60 min; t_{R}−2=14.56 min; Sample Solvent: EtOH:DCM=1:1 (v/v); Injection Volume: 0.9 mL) to afford tert-butyl 4-{7-[(2S)-2-[(tert-butoxycarbonyl)amino]-2-(3-methoxyphenyl) acetamido]-1H-indol-3-yl}pyrazole-1-carboxylate (180 mg, 63.74%) as a yellow solid. LCMS: m/z (ESI), [M+H−^{t}Bu]⁺=506.25. ¹H NMR (CD₃OD, 400 MHZ) δ 1.52 (9H, s), 1.71 (9H, s), 3.84 (3H, s), 5.51 (1H, s), 6.95 (1H, d), 7.14 (4H, dd), 7.34 (1H, t), 7.63 (1H, s), 7.67 (1H, m), 8.16 (1H, d), 8.43 (1H, s).

Step 2: Into a 100 mL round-bottom flask were added tert-butyl 4-{7-[(2S)-2-[(tert-butoxycarbonyl)amino]-2-(3-methoxyphenyl) acetamido]-1H-indol-3-yl}pyrazole-1-carboxylate (180 mg, 0.320 mmol, 1 equiv) and TFA (1 mL) and DCM (3 mL) at room temperature. The resulting mixture was stirred for 1 h at room temperature under air atmosphere. The resulting mixture was concentrated under reduced pressure. The residue was dissolved in DMF (2 mL). The mixture was basified to pH 9 with NH₃ (aq). The resulting mixture was stirred for 30 min at 0° C. under air atmosphere. The crude product (100 mg) was purified by prep-HPLC with the following conditions (Column: XBridge Prep OBD C18 30*150 mm, 5 um; Mobile Phase A: water (10 mmol/L NH₄HCO₃), Mobile Phase B: ACN; Flow rate: 60 mL/min; Gradient: 15% B to 45% B in 7 min; wavelength: 220 nm; t_{R}=6.92 min) to afford (2S)-2-amino-2-(3-methoxyphenyl)-N-[3-(1H-pyrazol-4-yl)-1H-indol-7-yl]acetamide (61.6 mg, 52.95%) as a white solid. LCMS: m/z (ESI), [M+H]⁺=362.15. ¹H NMR (DMSO-d₆, 400 MHZ) δ 3.71 (3H, s), 4.61 (1H, s), 6.84 (1H, m), 7.02 (1H, td), 7.10 (2H, m), 7.27 (1H, t), 7.37 (1H, d), 7.58 (2H, t), 7.96 (2H, s). SFC: t_{R}=1.460 min, ee=94.14%.

### Example 42

### Preparation of (2R)-2-amino-2-(3-methoxyphenyl)-N-[3-(1H-pyrazol-4-yl)-1H-indol-7-yl]acetamide

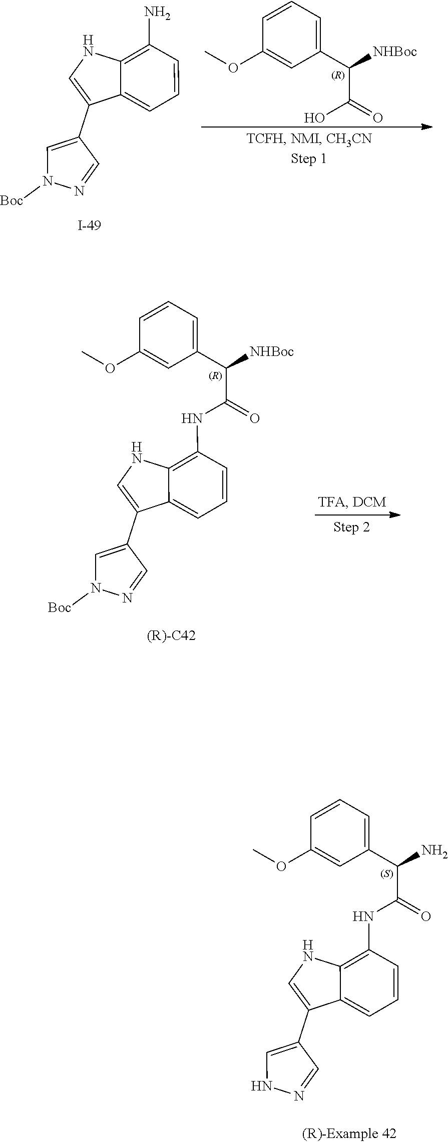

Step 1: Into a 20 mL vial were added amine I-49 (150 mg, 0.50 mmol, 1 equiv) and (R)-[(tert-butoxycarbonyl)amino](3-methoxyphenyl) acetic acid (155.58 mg, 0.55 mmol, 1.1 equiv) and TCFH (169.28 mg, 0.60 mmol, 1.2 equiv) and NMI (165.12 mg, 2.01 mmol, 4 equiv) and CH₃CN (5 mL) at room temperature. The resulting mixture was stirred for 40 min at room temperature under nitrogen atmosphere. The resulting mixture was concentrated under reduced pressure. The residue was purified by prep-TLC with CH₂Cl₂/methanol (25:1, v/v) to afford a crude solid. The crude product (280 mg) was purified by prep-chiral HPLC with the following conditions (Column: CHIRALPAK IE, 2*25 cm, 5 um; Mobile Phase A: Hex (0.2% DEA), Mobile Phase B: EtOH/DCM (1:1, v/v); Flow rate: 20 mL/min; Gradient: 30% B to 30% B in 18 min; wavelength: 220/254 nm; t_{R}−1=10.60 min; t_{R}−2=14.56 min; Sample Solvent: EtOH/DCM (1:1, v/v); Injection Volume: 0.9 mL) to afford tert-butyl 4-{7-[(2R)-2-[(tert-butoxycarbonyl)amino]-2-(3-methoxyphenyl) acetamido]-1H-indol-3-yl}pyrazole-1-carboxylate (240 mg, 84.99%) as a yellow solid. LCMS: m/z (ESI), [M+H−^{t}Bu]⁺=506.25. ¹H NMR (MeOH-d₄, 400 MHz) δ 1.52 (9H, s), 1.71 (9H, s), 3.84 (3H, s), 5.39 (1H, s), 7.14 (4H, m), 7.34 (2H, t), 7.63 (1H, s), 7.67 (1H, d), 8.16 (1H, s), 8.43 (1H, s).

Step 2: Into a 100 mL round-bottom flask were added tert-butyl 4-{7-[(2R)-2-[(tert-butoxycarbonyl)amino]-2-(3-methoxyphenyl) acetamido]-1H-indol-3-yl}pyrazole-1-carboxylate (240 mg, 0.43 mmol, 1 equiv) and TFA (1 mL) and DCM (3 mL) at room temperature. The resulting mixture was stirred for 1 h at room temperature under air atmosphere. The resulting mixture was concentrated under reduced pressure. The residue was dissolved in DMF (2 mL). The mixture was basified to pH 9 with NH₃ aq. The resulting mixture was stirred for 30 min at 0° C. under air atmosphere. The crude product (120 mg) was purified by prep-HPLC with the following conditions (Column: XBridge Prep OBD C18 30*150 mm, 5 um; Mobile Phase A: water (10 mmol/L NH₄HCO₃), Mobile Phase B: ACN; Flow rate: 60 mL/min; Gradient: 15% B to 45% B in 7 min; wavelength: 220 nm; t_{R}=7.02 min) to afford (2R)-2-amino-2-(3-methoxyphenyl)-N-[3-(1H-pyrazol-4-yl)-1H-indol-7-yl]acetamide (63.5 mg, 41.12%) as a white solid. LCMS: m/z (ESI), [M+H]⁺=362.15. ¹H NMR (DMSO-d₆, 400 MHZ) δ 3.75 (3H, d), 4.61 (1H, d), 6.84 (1H, dq), 7.02 (1H, td), 7.11 (2H, m), 7.27 (1H, td), 7.38 (1H, dd), 7.58 (2H, q), 7.95 (2H, s). SFC: t_{R}=1.110 min, ee=100%.

### Example 43 (Racemic), Example 44 (Enantiomer 1), and Example 45 (Enantiomer 2)

### Preparation of N-(3-(1H-pyrazol-4-yl)-1H-indol-7-yl)-2-(1-methylpiperidin-4-yl)-2-phenylacetamide

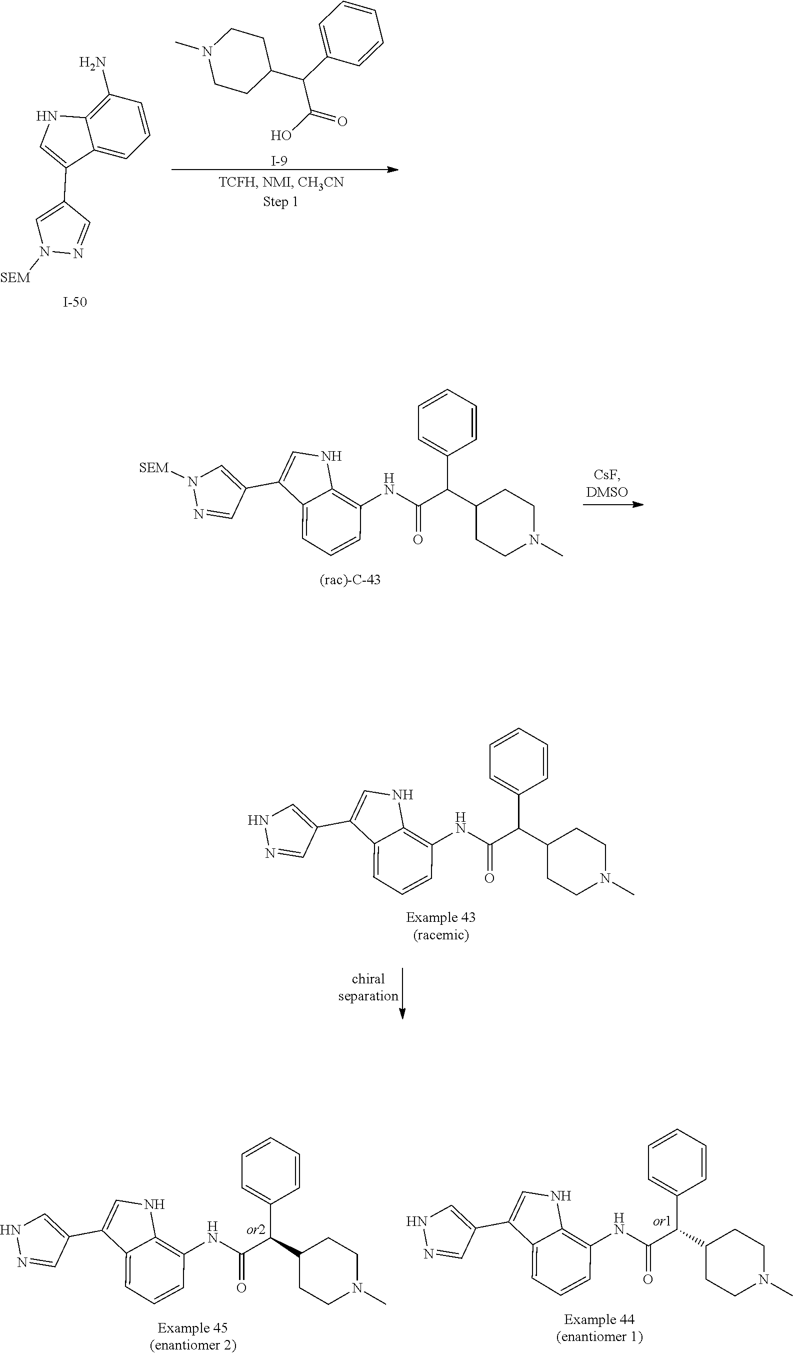

Step 1: Into a 10 mL vial were added amine I-50 (350 mg, 1.07 mmol, 1 equiv) and acid I-9 (248.59 mg, 1.07 mmol, 1 equiv) and TCFH (358.74 mg, 1.28 mmol, 1.2 equiv) and NMI (349.93 mg, 4.26 mmol, 4 equiv) in MeCN (8 mL) at room temperature. The resulting mixture was stirred for 2 h at room temperature under nitrogen atmosphere. The resulting mixture was concentrated under reduced pressure. The residue was purified by prep-TLC with CH₂Cl₂/methanol (10:1, v/v) to afford a crude solid (220 mg). The crude product (220 mg) was purified by prep-HPLC with the following conditions (Column: XBridge Prep OBD 30*150 mm, 5 um; Mobile Phase A: water (10 mmol/L NH₄HCO₃), Mobile Phase B: ACN; Flow rate: 60 mL/min; Gradient: 15% B to 45% B in 7 min; wavelength: 220 nm; t_{R}=7.65 min) to afford racemic C-43 (140 mg, 28.22%) as an off-white oil. LCMS: m/z (ESI), [M+H]⁺=544.50.

Step 2: Into a 8 mL vial were added racemic C-43 (70 mg, 0.13 mmol, 1 equiv) and CsF (58.66 mg, 0.39 mmol, 3 equiv) and DMSO (2.3 mL) at room temperature. The resulting mixture was stirred for 12 h at 100° C. under air atmosphere. The crude product was purified by Prep-HPLC with the following conditions (Column: XBridge Shield RP18 OBD Column, 30*150 mm, 5 um; Mobile Phase A: water (10 mmol/L NH₄HCO₃+0.1% NH₃ aq), Mobile Phase B: ACN; Flow rate: 60 mL/min; Gradient: 23% B to 48% B in 8 min; wavelength: 220 nm; t_{R}=7.37 min) to afford Example 43, racemic (22 mg) as a white solid. The solid was purified by prep-Chiral HPLC with the following conditions (Column: (R, R)-WHELK-01-Kromasi, 5*25 cm, 5 um; Mobile Phase A: Hex (0.2% DEA), Mobile Phase B: EtOH:DCM (1:1, v/v); Flow rate: 20 mL/min; Gradient: 50% B to 50% B in 10 min; wavelength: 220/254 nm; t_{R}−1=5.70 min; t_{R}−2=7.69 min; Sample Solvent: EtOH:DCM (1:1, v/v); Injection Volume: 0.7 mL) to afford Example 45, enantiomer 2 (9.5 mg, 17.78%) as a white solid. LCMS: m/z (ESI), [M+H]⁺=414.25. ¹H NMR (DMSO-d₆, 400 MHz) δ 0.85 (1H, d), 1.19 (2H, m), 1.44 (1H, d), 1.85 (1H, d), 2.08 (2H, s), 2.28 (3H, d), 2.70 (1H, m), 2.90 (1H, s), 3.50 (1H, d), 6.98 (1H, t), 7.27 (1H, t), 7.36 (2H, t), 7.49 (2H, s), 7.57 (3H, m), 7.95 (2H, s), 9.92 (1H, s), 10.72 (1H, s), 12.77 (1H, s). Chiral HPLC: t_{R}=1.743 min, ee=100% and Example 44, enantiomer 1 (5.4 mg, 9.82%) as a white solid. LCMS: m/z (ESI), [M+H]⁺=414.20. ¹H NMR (DMSO-d₆, 400 MHZ) δ 1.13 (3H, m), 1.47 (1H, d), 1.87 (1H, d), 2.00 (2H, m), 2.31 (3H, s), 2.92 (2H, d), 3.52 (1H, s), 6.98 (1H, t), 7.18 (1H, m), 7.28 (2H, m), 7.36 (2H, d), 7.52 (3H, m), 7.95 (2H, s), 9.95 (1H, s), 10.76 (1H, s), 12.77 (1H, s). Chiral HPLC: t_{R}=2.636 min, ee=99.06%.

### Example 46 (Racemic)

### Preparation of 2-(4-methylpiperazin-1-yl)-2-phenyl-N-[3-(1H-pyrazol-4-yl)-1H-indol-7-yl]acetamide

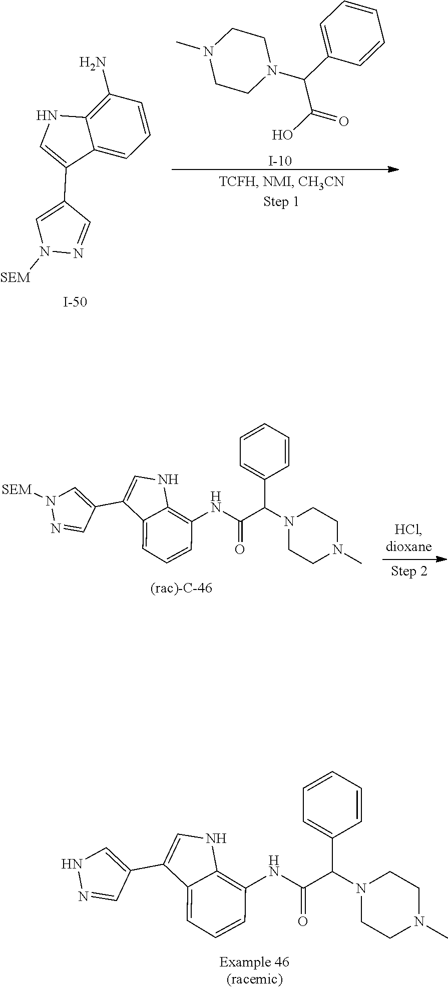

Step 1: Into a 40 mL vial were added amine I-50 (300 mg, 0.91 mmol, 1 equiv), NMI (374.92 mg, 4.57 mmol, 5 equiv), TCFH (512.49 mg, 1.83 mmol, 2 equiv), CH₃CN (10 mL) and acid I-10 (320.97 mg, 1.37 mmol, 1.5 equiv) at room temperature. The mixture was stirred for 12 h. The residue was purified by prep-TLC with CH₂Cl₂/methanol (20:1, v/v) to afford racemic amide C-46 (500 mg, 99.01%) as a light brown solid. LCMS: m/z (ESI), [M+H]⁺=545.25.

Step 2: Into a 50 mL round-bottom flask were added racemic amide C-46 (200 mg, 0.37 mmol, 1 equiv) and HCl (gas) in 1,4-dioxane (20 mL) at rt. The resulting mixture was stirred at 60° C. for 1 h under nitrogen atmosphere. The mixture was allowed to cool down to 0° C. and the mixture was basified to pH 9 with saturated NaHCO₃ (aq). The resulting mixture was extracted with CH₂Cl₂ (3×30 mL). The combined organic layer was dried over anhydrous Na₂SO₄. After filtration, the filtrate was concentrated under reduced pressure. The crude product (120 mg) was purified by prep-HPLC with the following conditions (Column: Xselect CSH C18 OBD 30*150 mm 5 um; Mobile Phase A: water (10 mmol/L NH₄HCO₃), Mobile Phase B: ACN; Flow rate: 25 mL/min; Gradient: 51% B to 64% B in 11 min; wavelength: 220 nm; t_{R}=10.3 min) to afford racemic Example 46 (2 mg, 1.27%) as a white solid. LCMS: m/z (ESI), [M+H]⁺=415.20. ¹H NMR (400 MHZ, CD₃OD) δ 2.33 (3H, s), 2.61 (8H, s), 4.15 (1H, s), 7.08 (1H, t), 7.17 (1H, dd), 7.42 (4H, m), 7.63 (3H, m), 7.91 (2H, s).

### Example 47 (Enantiomer 1) and Example 48 (Enantiomer 2)

### Preparation of N-(3-(1H-pyrazol-4-yl)-1H-indol-7-yl)-2-(4-methylpiperazin-1-yl)-2-phenylacetamide

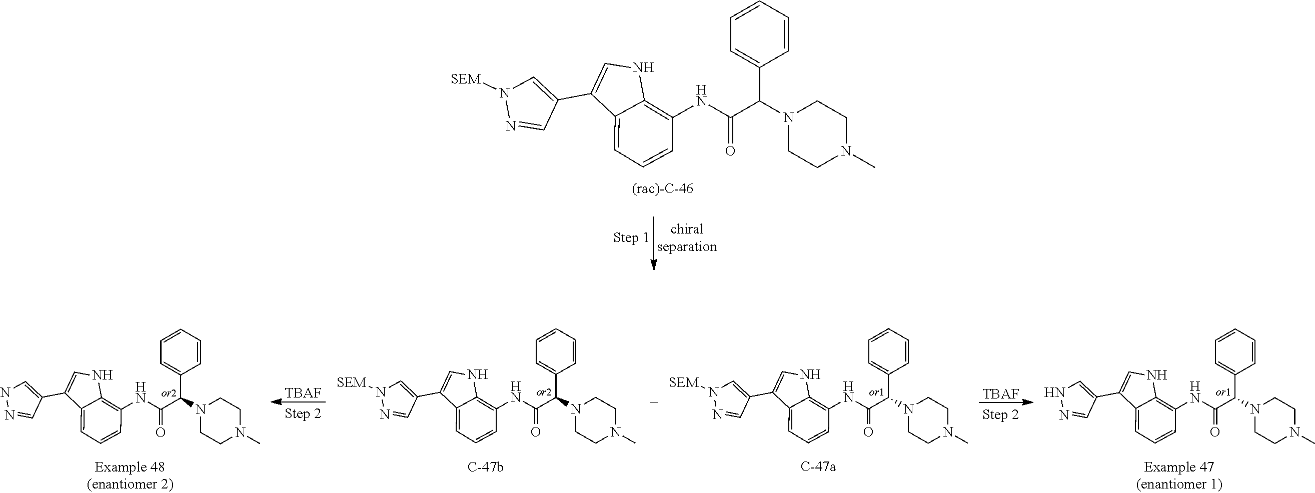

Step 1: The racemic amide C-46 (330 mg) was purified by prep-Chiral HPLC with the following conditions (Column: CHIRALPAK IF, 2*25 cm, 5 um; Mobile Phase A: Hex (0.2% DEA), Mobile Phase B: EtOH:DCM (1:1, v/v); Flow rate: 20 mL/min; Gradient: 30% B to 30% B in 12 min; wavelength: 220/254 nm; t_{R}−1=8.67 min; t_{R}−2=10.09 min; Sample Solvent: EtOH:DCM (1:1, v/v); Injection Volume: 0.25 mL) to afford enantiomer C-47b (124 mg, 37.6%), SFC: t_{R}=2.052 min, ee=100%, and enantiomer C-47a (110 mg, 33.3%) as a white solid, SFC: t_{R}=2.366 min, ee=99.02%.

Step 2: Into a 8 mL vial were added enantiomer C-47b (100 mg, 0.18 mmol, 1 equiv) and TBAF (2 mL, 0.008 mmol, 0.04 equiv) at room temperature. The resulting mixture was stirred for 8 h at 40° C. The residue was purified by reverse flash chromatography with the following conditions: column, C18 silica; mobile phase, MeCN in water, 10% to 50% gradient in 10 min; detector, UV 254 nm. The crude product (50 mg) was purified by prep-HPLC with the following conditions (Column: YMC-Actus Triart C18 ExRS, 30*150 mm, 5 um; Mobile Phase A: water (10 mmol/L NH₄HCO₃), Mobile Phase B: ACN; Flow rate: 60 mL/min; Gradient: 20% B to 45% B in 7 min; wavelength: 220 nm; t_{R}=6.53 min) to afford Example 48, enantiomer 2 (4.1 mg, 5.34%) as a white solid. LCMS: m/z (ESI), [M+H]⁺=415.25. ¹H NMR (400 MHZ, CD₃OD) δ 2.33 (s, 3H), 2.61 (s, 8H), 4.15 (s, 1H), 7.08 (t, 1H), 7.17 (dd, 1H), 7.42 (m, 4H), 7.63 (m, 3H), 7.91 (s, 2H). SFC: t_{R}=2.264 min, ee=97%.

Step 3: Into a 20 mL vial were added enantiomer C-47a (100 mg, 0.18 mmol, 1 equiv), DCM (4 mL) and HCl (gas) in 1,4-dioxane (4 mL) at room temperature. The resulting mixture was stirred for 8 h at 40° C. The resulting mixture was concentrated under reduced pressure. The mixture was basified to pH 9 with NH₃ (aq). The crude product (60 mg) was purified by prep-HPLC with the following conditions (Column: Xselect CSH C18 OBD 30*150 mm 5 um; Mobile Phase A: water (0.1% FA), Mobile Phase B: ACN; Flow rate: 60 mL/min; Gradient: 10% B to 22% B in 7 min; wavelength: 254; 220 nm; t_{R}=6.5 min) to afford Example 47, enantiomer 1 (17.9 mg, 22.84%) as a white solid. LCMS: m/z (ESI), [M+H]⁺=415.25. ¹H NMR (400 MHZ, CD₃OD) δ 2.33 (3H, s), 2.61 (4H, s), 3.16 (4H, s), 4.15 (1H, s), 7.08 (1H, t), 7.17 (1H, dd), 7.42 (4H, m), 7.63 (3H, m), 7.91 (2H, s), 8.49 (1H, s). SFC: t_{R}=2.074 min, ee=99.5%.

### Example 49 (Racemic), Example 50 (Enantiomer 1), and Example 51 (Enantiomer 2)

### Preparation of 3-(1-((3-(1H-pyrazol-4-yl)-1H-indol-7-yl)amino)-3-amino-1-oxopropan-2-yl)benzyl methylcarbamate

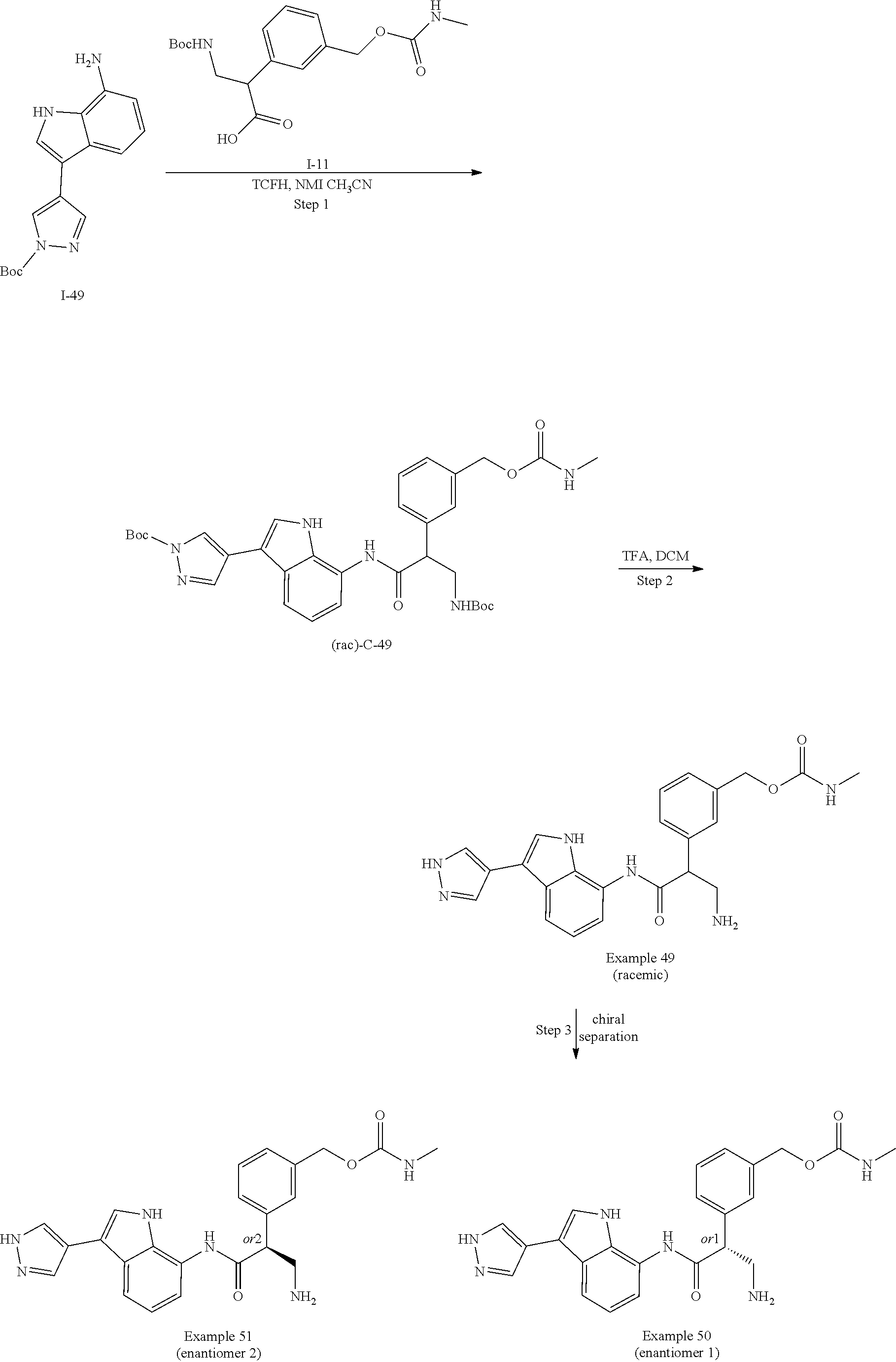

Step 1: To a stirred solution of acid I-11 (60 mg, 0.17 mmol, 1 equiv) and amine I-49 (60.96 mg, 0.20 mmol, 1.2 equiv) in CH₃CN (12 mL) were added NMI (13.98 mg, 0.17 mmol, 1 equiv) and TCFH (57.33 mg, 0.204 mmol, 1.2 equiv) at room temperature under air atmosphere. The resulting mixture was concentrated under reduced pressure. The resulting mixture was stirred for 2 h at room temperature under air atmosphere. The residue was purified by prep-TLC with PE/EA (1:1, v/v) to afford racemic amide C-49 (80 mg, 74.26% yield) as a yellow solid. LCMS: m/z (ESI), [M+H]⁺=633.35.

Step 2: To a stirred solution of racemic amide C-49 (80 mg, 0.13 mmol, 1 equiv) in DCM (5 mL) was added TFA (1 mL) at room temperature under air atmosphere. The resulting mixture was stirred for 2 h at room temperature under air atmosphere. The reaction was concentrated under reduced pressure, dissolved in methanol. The residue basified to pH 8 with NH₃ (aq). The crude product was purified by prep-HPLC with the following conditions (Column: Xselect CSH C18 OBD 30*150 mm 5 um; Mobile Phase A: water (0.1% FA), Mobile Phase B: ACN; Flow rate: 60 mL/min; Gradient: 10% B to 22% B in 7 min; wavelength: 254; 220 nm; t_{R}=6.03 min) to afford Example 49, racemic (29.1 mg, 48.10% yield) as a white solid. LCMS: m/z (ESI), [M+H]⁺=433.25. ¹H NMR: (MeOH-d₄, 400 MHZ) δ 2.70 (3H, s), 3.36 (1H, s), 3.67 (1H, dd), 4.26 (1H, dd), 5.14 (2H, s), 7.02-7.12 (2H, m), 7.42 (1H, d), 7.48 (3H, d), 7.52 (1H, s), 7.66 (1H, dd), 7.92 (2H, s), 8.5 (0.311H, s).

Step 3: The Example 49, racemic (188 mg) was purified by prep-HPLC with the following conditions (Column: CHIRALPAK IG, 2*25 cm, 5 um; Mobile Phase A: Hex (0.2% DEA)--HPLC, Mobile Phase B: EtOH:DCM=(1:1, v/v); Flow rate: 20 mL/min; Gradient: 50% B to 50% B in 10 min; wavelength: 220/254 nm; t_{R}−1=6.10 min; t_{R}−2=8.19 min; Sample Solvent: EtOH:DCM=(1:1, v/v); Injection Volume: 0.3 mL) to afford Example 51, enantiomer 2 (35.7 mg, 39.67%) after prep-HPLC (Column: XBridge Shield RP18 OBD Column, 30*150 mm, 5 um; Mobile Phase A: water (10 mmol/L NH₄HCO_{3+0.1}% NH₃·H₂O), Mobile Phase B: ACN; Flow rate: 60 mL/min; Gradient: 12% B to 40% B in 8 min, 40% B; wavelength: 220 nm; t_{R}=7.67 min) as a white solid. LCMS: m/z (ESI), [M+H]⁺=433.15. ¹H NMR: (CD₃OD, 400 MHZ) δ 2.69 (3H, s), 3.00 (1H, dd), 3.40 (1H, dd), 3.93 (1H, dd), 5.09 (2H, s), 6.97-7.09 (2H, m), 7.27-7.52 (5H, m), 7.62 (1H, dd), 7.89 (2H, s), SFC: t_{R}=1.580 min, ee=100%, and Example 50, enantiomer 1 (28.3 mg, 31.44%) after prep-HPLC (Column: XBridge Prep OBD C18 Column, 30*150 mm, 5 um; Mobile Phase A: water (10 mmol/L NH₄HCO₃), Mobile Phase B: ACN; Flow rate: 60 mL/min; Gradient: 5% B to 35% B in 7 min, 35% B; wavelength: 220 nm; t_{R}=8.08 min) as a white solid. LCMS: m/z (ESI), [M+H]⁺=433.15. ¹H NMR: (CD₃OD, 400 MHZ) δ 2.69 (3H, s), 3.00 (1H, dd), 3.40 (1H, dd), 3.93 (1H, dd), 5.09 (2H, s), 6.97-7.09 (2H, m), 7.27-7.52 (5H, m), 7.62 (1H, dd), 7.89 (2H, s). SFC: t_{R}=2.127 min, ee=97.47%.

### Example 52 (Racemic)

### Preparation of 3-amino-2-benzyl-N-[3-(1H-pyrazol-4-yl)-1H-indol-7-yl]propenamide

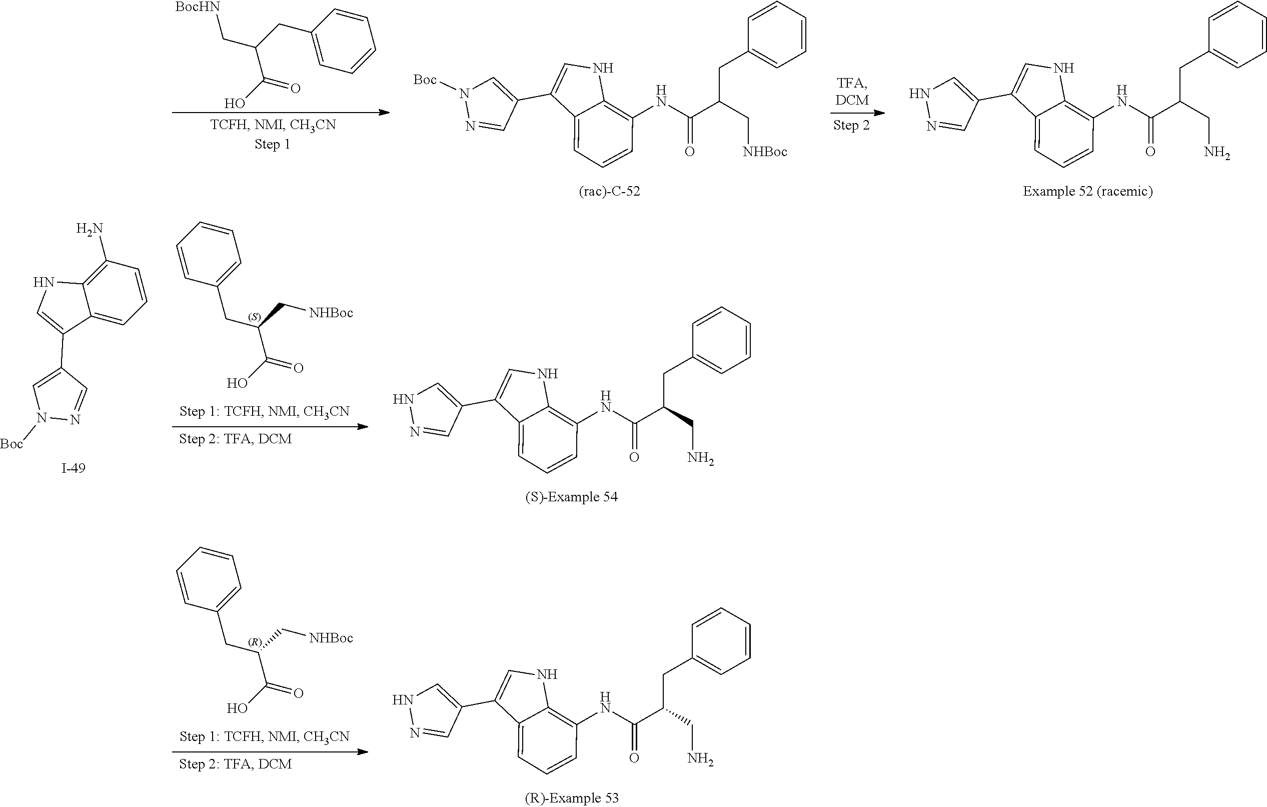

Step 1: Into a 20 mL vial were added amine I-49 (100 mg, 0.34 mmol, 1 equiv) and 2-benzyl-3-[(tert-butoxycarbonyl)amino]propanoic acid (140.44 mg, 0.50 mmol, 1.5 equiv), TCFH (141.07 mg, 0.50 mmol, 1.5 equiv) in NMI (137.60 mg, 1.68 mmol, 5.00 equiv) and MeCN (3 mL) at room temperature. The resulting mixture was stirred for 2 h at room temperature under nitrogen atmosphere. The resulting mixture was concentrated under reduced pressure. The residue was purified by prep-TLC with CH₂Cl₂/methanol (20:1, v/v) to afford tert-butyl 4-(7-{2-benzyl-3-[(tert-butoxycarbonyl)amino]propanamido}-1H-indol-3-yl) pyrazole-1-carboxylate (160 mg, 85.29%) as a off-white solid. LCMS: m/z (ESI), [M+H]⁺=560.2.

Step 2: Into a 20 mL round-bottom flask were added tert-butyl 4-(7-{2-benzyl-3-[(tert-butoxycarbonyl)amino]propanamido}-1H-indol-3-yl) pyrazole-1-carboxylate (150 mg, 0.27 mmol, 1 equiv) in DCM (3 mL) and TFA (1 mL) at room temperature. The resulting mixture was stirred for 1 h at room temperature under nitrogen atmosphere. The resulting mixture was concentrated under reduced pressure. The residue was dissolved in methanol (4 mL). The residue was basified to pH 9 with NH₃ (aq), and the resulting mixture was stirred at 0° C. for 30 min. The crude product (100 mg) was purified by prep-HPLC with the following conditions (Column: XBridge Shield RP18 OBD 30*150 mm, 5 um; Mobile Phase A: water (10 mmol/L NH₄HCO₃), Mobile Phase B: ACN; Flow rate: 60 mL/min; Gradient: 15% B to 40% B in 7 min; wavelength: 220 nm; t_{R}=6.28 min) to afford 3-amino-2-benzyl-N-[3-(1H-pyrazol-4-yl)-1H-indol-7-yl]propanamide (47 mg, 48.79%) as a white solid. LCMS: m/z (ESI), [M+H]⁺=360.2. ¹H NMR (CD₃OD, 400 MHZ) δ 2.80-3.05 (4H, m), 3.05-3.19 (1H, m), 6.90 (1H, dd), 7.03 (1H, t), 7.25 (1H, dt), 7.28-7.37 (4H, m), 7.43 (1H, d), 7.63 (1H, dd), 7.91 (2H, s).

### Example 53

### Preparation of (2R)-3-amino-2-benzyl-N-[3-(1H-pyrazol-4-yl)-1H-indol-7-yl]propanamide

Step 1: A solution of amine I-49 (120 mg, 0.402 mmol, 1 equiv) and (2R)-2-benzyl-3-[(tert-butoxycarbonyl)amino]propanoic acid (112.35 mg, 0.402 mmol, 1 equiv), TCFH (169.28 mg, 0.60 mmol, 1.5 equiv), NMI (115.59 mg, 1.41 mmol, 3.5 equiv) in MeCN (2 mL) was stirred for 1 h at room temperature under nitrogen atmosphere. The residue was purified by prep-TLC with CH₂Cl₂/methanol (30:1, v/v), the resulting mixture was concentrated under vacuum. The crude product (320 mg) was purified by prep-HPLC with the following conditions (Column: (R, R)-WHELK-O, 2.11*25 cm, 5 um; Mobile Phase A: Hex (0.2% DEA), Mobile Phase B: EtOH:DCM=1:1 (v/v); Flow rate: 20 mL/min; Gradient: 30% B to 30% B in 29 min; wavelength: 220/254 nm; t_{R}−1=18.45 min; t_{R}−2=22.38 min; Sample Solvent: EtOH:DCM=1:1 (v/v); Injection Volume: 1 mL) to afford tert-butyl 4-{7-[(2R)-2-benzyl-3-[(tert-butoxycarbonyl)amino]propanamido]-1H-indol-3-yl}pyrazole-1-carboxylate (140 mg, 62.19%) brown yellow solid. LCMS: m/z (ESI), [M+H]⁺=560.15. SFC: t_{R}=3.024 min, ee=100%.

Step 2: A solution of tert-butyl 4-{7-[(2R)-2-benzyl-3-[(tert-butoxycarbonyl)amino]propanamido]-1H-indol-3-yl}pyrazole-1-carboxylate (140 mg, 0.25 mmol, 1 equiv) and TFA (1 mL) in DCM (3 mL) was stirred for 1 h at room temperature under nitrogen atmosphere. The resulting mixture was concentrated under reduced pressure. The residue was dissolved in methanol (2 mL). The mixture was basified to pH 9 with NH₃ aq at 0° C. The resulting mixture was stirred for 30 min at 0° C. under nitrogen atmosphere. The crude product (100 mg) was purified by Prep-HPLC with the following conditions (Column: XBridge Shield RP18 OBD Column, 30*150 mm, 5 um; Mobile Phase A: water (10 mmol/L NH₄HCO₃), Mobile Phase B: ACN; Flow rate: 60 mL/min; Gradient: 12% B to 42% B in 8 min; wavelength: 220 nm; t_{R}=7.50 min) to afford (2R)-3-amino-2-benzyl-N-[3-(1H-pyrazol-4-yl)-1H-indol-7-yl]propanamide (37.0 mg, 40.95%) as a off-white solid. LCMS: m/z (ESI), [M+H]⁺=360.30. ¹H NMR (CD₃OD, 400 MHZ) δ 2.86-3.05 (4H, m), 3.10-3.18 (1H, m), 6.90 (1H, d), 7.03 (1H, t), 7.23-7.29 (1H, m), 7.33 (4H, d), 7.43 (1H, s), 7.64 (1H, dd), 7.91 (2H, s). SFC: t_{R}=2.712 min, ee=100%.

### Example 54

### Preparation of (2S)-3-amino-2-benzyl-N-[3-(1H-pyrazol-4-yl)-1H-indol-7-yl]propanamide

Step 1: A solution of amine I-49 (110 mg, 0.37 mmol, 1 equiv) and (2S)-2-benzyl-3-[(tert-butoxycarbonyl)amino]propanoic acid (102.99 mg, 0.37 mmol, 1 equiv), TCFH (155.17 mg, 0.55 mmol, 1.5 equiv), NMI (105.95 mg, 1.29 mmol, 3.5 equiv) in MeCN (3 mL) was stirred for 1 h at room temperature under nitrogen atmosphere. The resulting mixture was concentrated under reduced pressure. The residue was purified by prep-TLC with CH₂Cl₂/methanol (30:1, v/v) to afford tert-butyl 4-{7-[(2S)-2-benzyl-3-[(tert-butoxycarbonyl)amino]propanamido]-1H-indol-3-yl}pyrazole-1-carboxylate (200 mg, 96.92%) as a light pink solid. LCMS: m/z (ESI), [M+H−^{t}Bu]⁺=504.25.

Step 2: A solution of tert-butyl 4-{7-[(2S)-2-benzyl-3-[(tert-butoxycarbonyl)amino]propanamido]-1H-indol-3-yl}pyrazole-1-carboxylate (200 mg, 0.36 mmol, 1 equiv) and TFA (1 mL) in DCM (3 mL) was stirred for 1 h at room temperature under nitrogen atmosphere. The resulting mixture was concentrated under reduced pressure. The residue was dissolved in methanol (3 mL). The mixture was basified to pH 9 with NH₃—H₂O at 0° C. The resulting mixture was stirred for 30 min at 0° C. under nitrogen atmosphere. The crude product (160 mg) was purified by prep-HPLC with the following conditions (Column: XBridge Shield RP18 OBD 30*150 mm, 5 um; Mobile Phase A: water (10 mmol/L NH₄HCO₃+0.1% NH₃ aq), Mobile Phase B: ACN; Flow rate: 60 mL/min; Gradient: 15% B to 42% B in 8 min; wavelength: 220 nm; t_{R}=7.72 min) to afford (2S)-3-amino-2-benzyl-N-[3-(1H-pyrazol-4-yl)-1H-indol-7-yl]propanamide (43.6 mg, 33.69%) as a off-white solid. LCMS: m/z (ESI), [M+H]⁺=360.10. ¹H NMR (CD₃OD, 400 MHZ) δ 2.88-3.03 (4H, m), 3.12 (1H, dd), 6.90 (1H, dd), 7.03 (1H, t), 7.23-7.29 (1H, m), 7.33 (4H, d), 7.43 (1H, s), 7.64 (1H, dd), 7.91 (2H, s). SFC: t_{R}=2.462 min, ee=93.97%.

### Example 55

### Preparation of(S)-3-amino-N-(3-(3-methyl-1H-pyrazol-4-yl)-1H-indol-7-yl)-2-phenylpropanamide

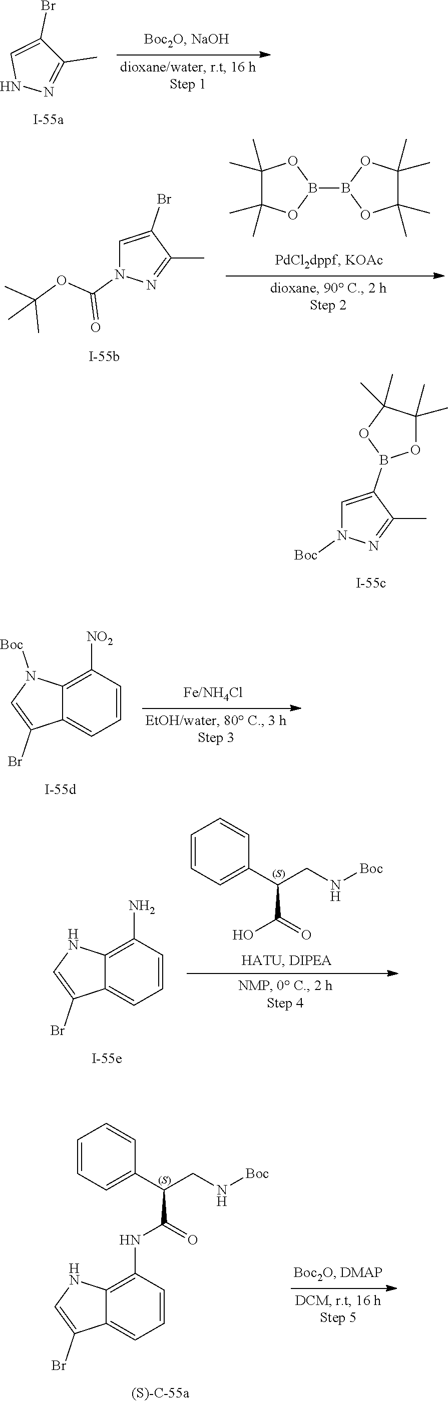

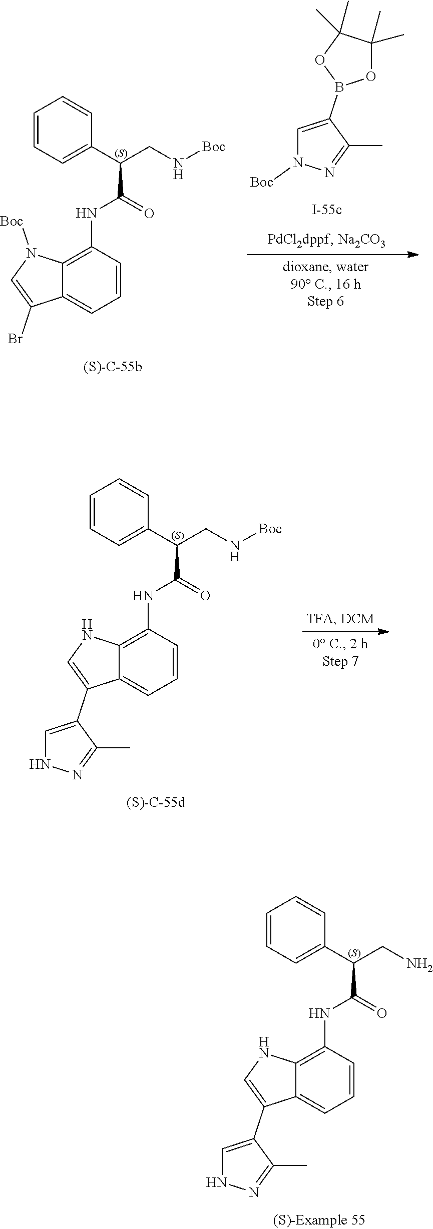

Step 1:4-bromo-3-methyl-1H-pyrazole I-55a (100 mg, 621.12 μmol), tert-butoxycarbonyl tert-butyl carbonate (216.89 mg, 993.79 μmol, 228.07 μL) and sodium hydroxide (27.33 mg, 683.23 μmol) were placed in water (1.50 mL)/dioxane (1.50 mL) and the reaction mixture was stirred at room temperature for 16 h. The solvent was removed and the residue treated with ethyl acetate and filtered. The filtrate was concentrated under reduced pressure to afford tert-butyl 4-bromo-3-methyl-pyrazole-1-carboxylate I-55b (160 mg, 99% yield) as brown solid. LCMS: t_{R}=1.80 min in 3 min chromatography (3 min-5-95% MeCN in water (0.02% TFA), Waters Acquity UPLC BEH C18 1.7 μm, 2.1*50 mm, 40° C.), MS (ESI) m/z=206.8 [M−55]⁺.

Step 2: A mixture of tert-butyl 4-bromo-3-methyl-pyrazole-1-carboxylate I-55b (160 mg, 612.76 μmol), 4,4,5,5-tetramethyl-2-(4,4,5,5-tetramethyl-1,3,2-dioxaborolan-2-yl)-1,3,2-dioxaborolane (186.72 mg, 735.31 μmol), potassium acetate (78.18 mg, 796.58 μmol) and PdCl₂dppf (50.04 mg, 61.28 μmol) in dioxane (2.00 mL) was stirred and heated at 90° C. for 2 h under nitrogen. The mixture was used for step 6 without further purification. LCMS: t_{R}=2.00 min in 3 min chromatography (3 min-5-95% MeCN in water (6 mmol/L NH₄HCO₃), Waters Acquity UPLC BEH C18 1.7 μm, 2.1*50 mm, 40° C.), MS (ESI) m/z=309.2 [M+H]⁺.

Step 3: To a mixture of tert-butyl 3-bromo-7-nitro-indole-1-carboxylate I-55d (820 mg, 2.40 mmol) and ammonia hydrochloride (1.29 g, 24.04 mmol) in ethanol (15 mL) and water (15 mL) was added iron powder (671.20 mg, 12.02 mmol, 85.39 μL). Then the reaction mixture was stirred and heated at 80° C. for 3 h. The reaction mixture was filtered through celite and washed with ethanol (50 mL×3), and the filtrate was concentrated. The residue was dissolved 200 ml of ethyl acetate, filtered and the filtration was concentrated under reduced pressure to afford 3-bromo-1H-indol-7-amine I-55e (393 mg, 77% yield) as brown solid. LCMS: t_{R}=1.25 min in 3 min chromatography (3 min-5-95% MeCN in water (6 mmol/L NH₄HCO₃), Warters Acquity UPLC BEH C18 1.7 μm, 2.1*50 mm, 40° C.), MS (ESI) m/z=210.9 [M+H]⁺.

Step 4:3-bromo-1H-indol-7-amine I-55e (393 mg, 1.02 mmol), (2S)-3-(tert-butoxycarbonylamino)-2-phenyl-propanoic acid (200 mg, 753.85 μmol) and DIPEA (292.28 mg, 2.26 mmol, 393.91 μL) in NMP (3.00 mL) were stirred at room temperature for 15 min. Then the reaction mixture was cooled at 0° C. by ice/water bath. Then HATU (374.60 mg, 980.01 μmol) was added at this temperature. The resulting suspension was stirred at 0° C. for 2 h. Then reaction mixture was poured into water (60 mL), and extracted with ethyl acetate (30 mL×3). The combined organic layers were washed with brine (80 mL×1), dried over sodium sulfate, filtered, and the filtrate was concentrated. The residue was purified by silica flash chromatography column, elution gradient from 15% to 50% ethyl acetate in petroleum ether. Pure fractions were evaporated to dryness to afford tert-butyl N-[(2S)-3-[(3-bromo-1H-indol-7-yl)amino]-3-oxo-2-phenyl-propyl]carbamate C-55a (280 mg, 81% yield) as light brown solid. LCMS: t_{R}=1.97 min in 3 min chromatography (3 min-5-95% MeCN in water (0.02% TFA), Watersarters Acquity UPLC BEH C18 1.7 μm, 2.1*50 mm, 40° C.), MS (ESI) m/z=458.1 [M+H]⁺.

Step 5: tert-butyl N-[(2S)-3-[(3-bromo-1H-indol-7-yl)amino]-3-oxo-2-phenyl-propyl]carbamate C-55a (242 mg, 527.98 μmol) was dissolved in DCM (5 mL). tert-butoxycarbonyl tert-butyl carbonate (126.75 mg, 580.78 μmol, 133.28 μL) and N,N-dimethylpyridin-4-amine (6.45 mg, 52.80 μmol) were added and the solution was stirred at 25° C. for 16 h. The reaction mixture was diluted with DCM (50 mL) and washed with water (30 mL×3). The organic layer was dried over anhydrous sodium sulfate, filtered and concentrated under reduced pressure to afford tert-butyl 3-bromo-7-[[(2S)-3-(tert-butoxycarbonylamino)-2-phenyl-propanoyl]amino]indole-1-carboxylate C-55b (270 mg, 92% yield) as brown solid. LCMS: t_{R}=2.35 min in 3 min chromatography (3 min-5-95% MeCN in water (0.02% TFA), Waters Acquity UPLC BEH C18 1.7 μm, 2.1*50 mm, 40° C.), MS (ESI) m/z=558.1 [M+H]⁺.

Step 6: tert-butyl 3-bromo-7-[[(2S)-3-(tert-butoxycarbonylamino)-2-phenyl-propanoyl]amino]indole-1-carboxylate C-55b (90 mg, 161.16 μmol), tert-butyl 3-methyl-4-(4,4,5,5-tetramethyl-1,3,2-dioxaborolan-2-yl) pyrazole-1-carboxylate I-55c (124.16 mg, 402.89 μmol), PdCl₂dppf (13.16 mg, 16.12 μmol) and sodium carbonate (51.24 mg, 483.47 μmol) in dioxane (2.00 mL) and water (0.40 mL) were stirred and heated at 90° C. for 16 h under nitrogen. The solvent was removed under reduced pressure. The residue was purified by silica column chromatography, elution gradient from 0 to 30% ethyl acetate in petroleum ether. Pure fractions were evaporated to dryness to afford tert-butyl N-[(2S)-3-[[3-(3-methyl-1H-pyrazol-4-yl)-1H-indol-7-yl]amino]-3-oxo-2-phenyl-propyl]carbamate C-55d (12 mg, 16% yield) as brown solid. LCMS: t_{R}=2.24 min in 3 min chromatography (3 min-5-95% MeCN in water (0.02% TFA), Waters Acquity UPLC BEH C18 1.7 μm, 2.1*50 mm, 40° C.), MS (ESI) m/z=460.2 [M+H]⁺.

Step 7: To a solution of tert-butyl N-[(2S)-3-[[3-(3-methyl-1H-pyrazol-4-yl)-1H-indol-7-yl]amino]-3-oxo-2-phenyl-propyl]carbamate C-55d (12 mg, 26.11 μmol) in DCM (0.30 mL) was stirred and cooled at 0° C. by ice/water bath. Then added trifluoroacetic acid (148.87 mg, 1.31 mmol, 100.59 μL) dropwise into the reaction. The resulting mixture was stirred at this temperature for 2 h. The reaction mixture was concentrated under reduced pressure. The residue was purified by C18-flash chromatography, elution gradient from 0% to 30% MeCN in water (0.02% TFA). Pure fractions were lyophilized to dryness to afford (2S)-3-amino-N-[3-(3-methyl-1H-pyrazol-4-yl)-1H-indol-7-yl]-2-phenyl-propanamide Example 55 (1.95 mg, 16% yield, TFA salt) as brown solid. LCMS: t_{R}=1.36 min in 3 min chromatography (3 min-5-95% MeCN in water (6 mmol/L NH₄HCO₃), Waters Acquity UPLC BEH C18 1.7 μm, 2.1*50 mm, 40° C.), MS (ESI) m/z=360.2 [M+H]⁺. ¹H NMR: (400 MHz, DMSO-d₆) δ 10.96 (s, 1H), 10.32 (s, 1H), 8.06 (s, 3H), 7.75 (s, 1H), 7.50 (d, J=7.3 Hz, 2H), 7.40 (d, J=8.0 Hz, 3H), 7.34 (d, J=7.1 Hz, 1H), 6.97 (t, J=7.8 Hz, 1H), 4.29 (dd, J=9.1, 4.6 Hz, 1H), 3.82-3.70 (m, 1H), 3.14 (dd, J=13.2, 6.8 Hz, 1H), 2.29 (s, 3H).

### Example 56

### Preparation of (2S)-3-amino-N-[3-(3,5-dimethyl-1H-pyrazol-4-yl)-1H-indol-7-yl]-2-phenyl-propanamide

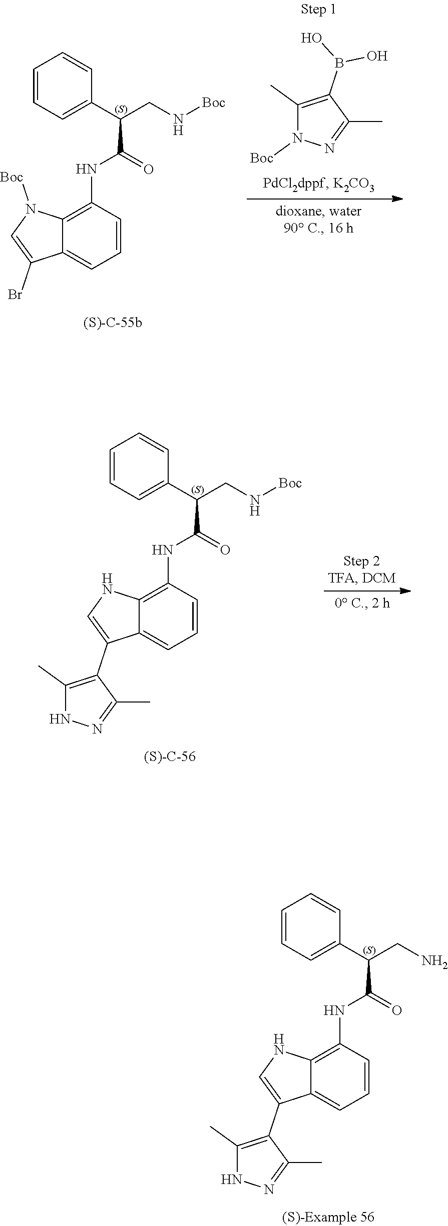

Step 1: tert-butyl 3-bromo-7-[[(2S)-3-(tert-butoxycarbonylamino)-2-phenyl-propanoyl]amino]indole-1-carboxylate C-55b (50 mg, 89.53 μmol), (1-tert-butoxycarbonyl-3,5-dimethyl-pyrazol-4-yl) boronic acid (32.24 mg, 134.30 μmol), K₂CO₃ (30.93 mg, 223.83 μmol, 13.51 μL) and PdCl₂dppf (6.55 mg, 8.95 μmol) in dioxane (2.00 mL) and water (0.40 mL) were stirred under nitrogen at 90° C. for 16 h. The solvent was removed under reduced pressure. The residue was purified by silica column chromatography, elution gradient from 0 to 10% methanol in DCM. Pure fractions were evaporated to dryness to afford tert-butyl N-[(2S)-3-[[3-(3,5-dimethyl-1H-pyrazol-4-yl)-1H-indol-7-yl]amino]-3-oxo-2-phenyl-propyl]carbamate C-56 (22 mg, 52% yield) as brown solid. LCMS: t_{R}=1.69 min in 3 min chromatography (3 min-5-95% MeCN in water (0.02% TFA), Warters Acquity UPLC BEH C18 1.7 μm, 2.1*50 mm, 40° C.), MS (ESI) m/z=474.4 [M+H]⁺.

Step 2: To a solution of tert-butyl N-[(2S)-3-[[3-(3,5-dimethyl-1H-pyrazol-4-yl)-1H-indol-7-yl]amino]-3-oxo-2-phenyl-propyl]carbamate C-56 (22 mg, 46.46 μmol) in DCM (0.60 mL) was cooled at 0° C. in ice/water bath, and stirred. Then added trifluoroacetic acid (264.85 mg, 2.32 mmol, 178.95 μL) dropwise into the reaction. The resulting mixture was stirred for 2 h. The reaction mixture was concentrated under reduced pressure. The residue was purified by C18-flash chromatography, elution gradient from 0% to 30% MeCN in water (0.02% TFA). Pure fractions were lyophilized to dryness to afford (2S)-3-amino-N-[3-(3,5-dimethyl-1H-pyrazol-4-yl)-1H-indol-7-yl]-2-phenyl-propanamide Example 56 (4 mg, 18% yield, TFA salt) as white solid. LCMS: t_{R}=1.47 min in 3 min chromatography (3 min-5-95% MeCN in water (6 mmol/L NH₄HCO₃), Waters Acquity UPLC BEH C18 1.7 μm, 2.1*50 mm, 40° C.), MS (ESI) m/z=374.3 [M+H]⁺. ¹H NMR: (400 MHZ, DMSO-d₆) δ 10.95 (d, J=1.1 Hz, 1H), 10.29 (s, 1H), 8.07 (s, 3H), 7.49 (d, J=7.2 Hz, 2H), 7.42 (t, J=7.5 Hz, 2H), 7.35 (dd, J=12.5, 4.9 Hz, 3H), 7.11 (d, J=7.9 Hz, 1H), 6.95 (t, J=7.8 Hz, 1H), 4.25 (dd, J=9.3, 5.0 Hz, 1H), 3.57 (ddd, J=14.6, 11.9, 5.6 Hz, 1H), 3.14 (dt, J=17.4, 5.6 Hz, 1H), 2.15 (s, 6H).

### Example 57

### Preparation of(S)-3-amino-N-(3-(3-fluoro-1H-pyrazol-4-yl)-1H-indol-7-yl)-2-phenylpropanamide

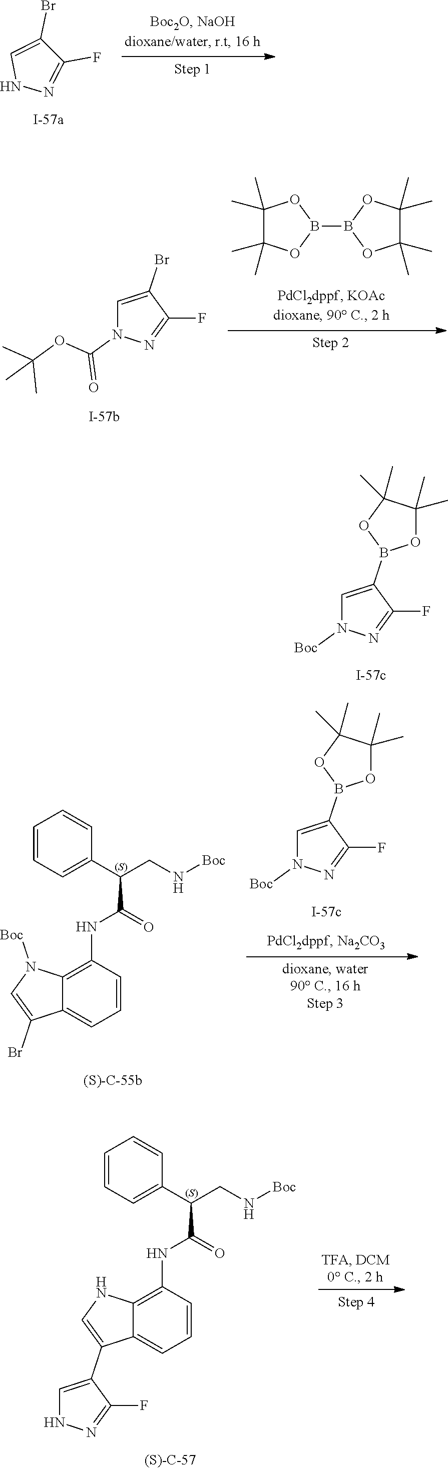

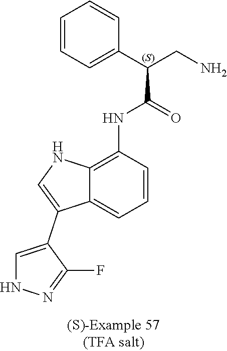

Step 1:4-bromo-3-fluoro-1H-pyrazole I-57a (100 mg, 606.19 μmol), tert-butoxycarbonyl tert-butyl carbonate (211.68 mg, 969.91 μmol, 222.59 μL) and sodium hydroxide (26.67 mg, 666.81 μmol) were placed in water (1.50 mL)/dioxane (1.50 mL) and the reaction mixture was stirred at room temperature for 16 h. The solvent was removed and the residue treated with ethyl acetate and filtered. The filtrate was concentrated under reduced pressure to afford tert-butyl 4-bromo-3-fluoro-pyrazole-1-carboxylate I-57b (160 mg, 99% yield) as brown solid. LCMS: t_{R}=1.84 min in 3 min chromatography (3 min-5-95% MeCN in water (0.02% TFA), Waters Acquity UPLC BEH C18 1.7 μm, 2.1*50 mm, 40° C.), MS (ESI) m/z=164.8 [M−100]⁺.

Step 2: A mixture of tert-butyl 4-bromo-3-fluoro-pyrazole-1-carboxylate I-57b (160 mg, 603.59 μmol), 4,4,5,5-tetramethyl-2-(4,4,5,5-tetramethyl-1,3,2-dioxaborolan-2-yl)-1,3,2-dioxaborolane (183.93 mg, 724.31 μmol), potassium acetate (77.01 mg, 784.67 μmol) and PdCl₂dppf (49.29 mg, 60.36 μmol) in dioxane (3.00 mL) was stirred and heated at 90° C. for 2 h under nitrogen. The mixture was used in step 3 without further purification. LCMS: t_{R}=1.88 min in 3 min chromatography (3 min-5-95% MeCN in water (6 mmol/L NH₄HCO₃), Waters Acquity UPLC BEH C18 1.7 μm, 2.1*50 mm, 40° C.), MS (ESI) m/z=257.1 [M−55]⁺.

Step 3: tert-butyl 3-bromo-7-[[(2S)-3-(tert-butoxycarbonylamino)-2-phenyl-propanoyl]amino]indole-1-carboxylate C-55b (90 mg, 161.16 μmol), tert-butyl 3-fluoro-4-(4,4,5,5-tetramethyl-1,3,2-dioxaborolan-2-yl) pyrazole-1-carboxylate I-57c (125.76 mg, 402.89 μmol), PdCl₂dppf (13.16 mg, 16.12 μmol) and sodium carbonate (51.24 mg, 483.47 μmol, 20.24 μL) in dioxane (2.00 mL) and water (0.40 mL) were stirred under nitrogen at 90° C. for 16 h. The solvent was removed under reduced pressure. The residue was purified by silica flash chromatography column, elution gradient from 0 to 30% ethyl acetate in petroleum ether. Pure fractions were evaporated to dryness to afford tert-butyl N-[(2S)-3-[[3-(3-fluoro-1H-pyrazol-4-yl)-1H-indol-7-yl]amino]-3-oxo-2-phenyl-propyl]carbamate C-57 (15 mg, 20% yield) as brown solid. LCMS: t_{R}=2.21 min in 3 min chromatography (3 min-5-95% MeCN in water (0.02% TFA), Waters Acquity UPLC BEH C18 1.7 μm, 2.1*50 mm, 40° C.), MS (ESI) m/z=464.2 [M+H]⁺.

Step 4: To a solution of tert-butyl N-[(2S)-3-[[3-(3-fluoro-1H-pyrazol-4-yl)-1H-indol-7-yl]amino]-3-oxo-2-phenyl-propyl]carbamate C-57 (15 mg, 26.02 μmol, TFA) in DCM (0.30 mL) was stirred and cooled at 0° C. by ice/water bath. Then added trifluoroacetic acid (148.33 mg, 1.30 mmol, 100.23 μL) dropwise into the reaction. The resulting mixture was stirred at this temperature for 2 h. The reaction mixture was concentrated under reduced pressure. The residue was purified by C18-flash chromatography column, elution gradient from 0% to 30% MeCN in water (0.02% TFA). Pure fractions were lyophilized to dryness to afford (2S)-3-amino-N-[3-(3-fluoro-1H-pyrazol-4-yl)-1H-indol-7-yl]-2-phenyl-propanamide Example 57 (1.85 mg, 20% yield, TFA salt) as off-white solid. LCMS: t_{R}=1.47 min in 3 min chromatography (3 min-5-95% MeCN in water (6 mmol/L NH₄HCO₃), Warters Acquity UPLC BEH C18 1.7 μm, 2.1*50 mm, 40° C.), MS (ESI) m/z=364.2 [M+H]⁺. ¹H NMR: (400 MHz, DMSO-d₆) δ 12.44 (s, 1H), 11.17 (s, 1H), 10.39 (s, 1H), 8.08 (s, 1H), 7.53-7.47 (m, 5H), 7.41 (t, J=7.3 Hz, 3H), 7.37-7.29 (m, 2H), 7.01 (t, J=7.8 Hz, 1H), 4.31 (qdd, J=6.1, 4.4, 2.8 Hz, 1H), 3.61-3.53 (m, 1H), 3.14-3.09 (m, 1H).

### Example 58 (Enantiomer 1) and Example 59 (Enantiomer 2)

### Preparation of N-(3-(1H-pyrazol-4-yl)-1H-indol-7-yl)-3-amino-2-(4-cyanophenyl)propanamide

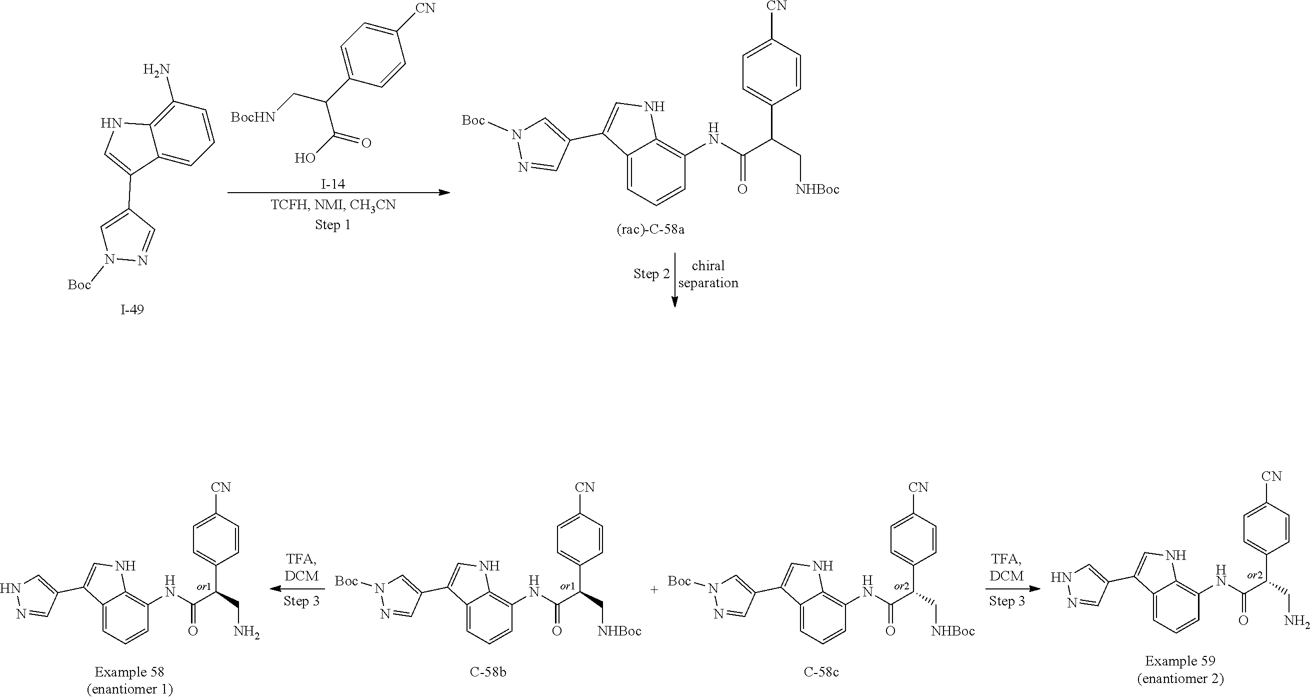

Step 1: To a stirred mixture of acid I-14 (400 mg, 1.38 mmol, 1 equiv) in CH₃CN (8.0 mL) was added NMI (339.38 mg, 4.13 mmol, 3.0 equiv), amine I-49 (419.28 mg, 1.41 mmol, 1.02 equiv) and TCFH (773.16 mg, 2.76 mmol, 2.0 equiv) in portion at 25° C. under air atmosphere. The resulting mixture was stirred for overnight at 25° C. under air atmosphere. The resulting mixture was diluted with water (10 mL). The resulting mixture was extracted with ethyl acetate (3×30 mL). The combined organic layers were washed with water (3×10 mL), dried over anhydrous Na₂SO₄. After filtration, the filtrate was concentrated under reduced pressure. The residue was purified by prep-TLC with CH₂Cl₂/methanol (20:1, v/v) to afford racemic amide C-58a (450 mg, 57.23%) as a yellow oil. LCMS: m/z (ESI), [M-Boc-^{t}Bu]⁺=471.25.

Step 2: The racemic amide C-58a (450 mg, 0.79 mmol, 1.0 equiv) was separated with the following condition (Column: CHIRALPAK IG, 2*25 cm, 5 um; Mobile Phase A: Hex (0.2% DEA), Mobile Phase B: EtOH:DCM (1:1, v/v); Flow rate: 20 mL/min; Gradient: 40% B to 40% B in 12 min; wavelength: 220/254 nm; t_{R}−1=3.71 min; t_{R}−2=6.23 min; Sample Solvent: EtOH:DCM (1:1, v/v); Injection Volume: 0.55 mL) to afford enantiomer C-58b (200 mg, 44.4%), SFC: t_{R}=1.488 min, ee=100%, and enantiomer C-58c (135 mg, 30.0%) as a white solid, SFC: t_{R}=2.431 min, ee=100%.

Step 3: To a stirred solution of enantiomer C-58b (200 mg, 0.35 mmol, 1 equiv) in DCM (5 mL) was added TFA (1 mL, 13.46 mmol) at room temperature under air atmosphere. The resulting mixture was stirred for 2 h at room temperature under air atmosphere. The reaction was quenched by the addition of water (5 mL) at room temperature. The resulting mixture was extracted with CH₂Cl₂ (3×10 mL). The combined organic layers were dried over anhydrous Na₂SO₄. After filtration, the filtrate was concentrated under reduced pressure. The mixture was basified to pH 9 with NH₃—H₂O. The crude product was purified by prep-HPLC with the following conditions (Column: XBridge Prep OBD C18 30*150 mm, 5 um; Mobile Phase A: water (10 mmol/L NH₄HCO₃), Mobile Phase B: ACN; Flow rate: 60 mL/min; Gradient: 18% B to 31% B in 8 min; wavelength: 254 nm; t_{R}=7.65 min) to afford Example 58, enantiomer 1 (26.6 mg, 20.49%) as a white solid. LCMS: m/z (ESI), [M+H]⁺=371.15. ¹H NMR (CD₃OD, 400 MHZ) δ 3.03 (1H, dd), 3.43 (1H, dd), 4.02 (1H, dd), 7.06 (2H, m), 7.47 (1H, s), 7.67 (3H, m), 7.77 (2H, dd), 7.92 (2H, s). Chiral HPLC: t_{R}=2.574 min, ee=97.7%.

To a stirred solution of enantiomer C-58c (135 mg, 0.24 mmol, 1 equiv) in DCM (5 mL) was added TFA (1 mL, 13.46 mmol) at room temperature under air atmosphere. The resulting mixture was stirred for 2 h at room temperature under air atmosphere. The reaction was quenched by the addition of water (10 mL) at room temperature. The resulting mixture was extracted with CH₂Cl₂ (3×10 mL). The combined organic layers were dried over anhydrous Na₂SO₄. After filtration, the filtrate was concentrated under reduced pressure. The mixture was basified to pH 9 with NH₃ (aq). The crude product was purified by prep-HPLC with the following conditions (Column: XBridge Prep OBD C18 30*150 mm, 5 um; Mobile Phase A: water (10 mmol/L NH₄HCO₃), Mobile Phase B: ACN; Flow rate: 60 mL/min; Gradient: 5% B to 40% B in 7 min; wavelength: 220 nm; t_{R}=8.05 min) to afford Example 59, enantiomer 2 (31.5 mg, 35.95%) as a white solid. LCMS: m/z (ESI), [M+H]⁺=371.15. ¹H NMR (CD₃OD, 400 MHZ) δ 3.05 (1H, dd), 3.45 (1H, dd), 4.04 (1H, dd), 7.06 (2H, m), 7.47 (1H, s), 7.66 (3H, m), 7.78 (2H, m), 7.92 (2H, s). Chiral HPLC: t_{R}=3.535 min, ee=95.8%.

### Example 60 (Enantiomer 1) and Example 61 (Enantiomer 2)

### Preparation of N-(3-(1H-pyrazol-4-yl)-1H-indol-7-yl)-3-amino-2-(6-(hydroxymethyl)pyridin-3-yl)propanamide

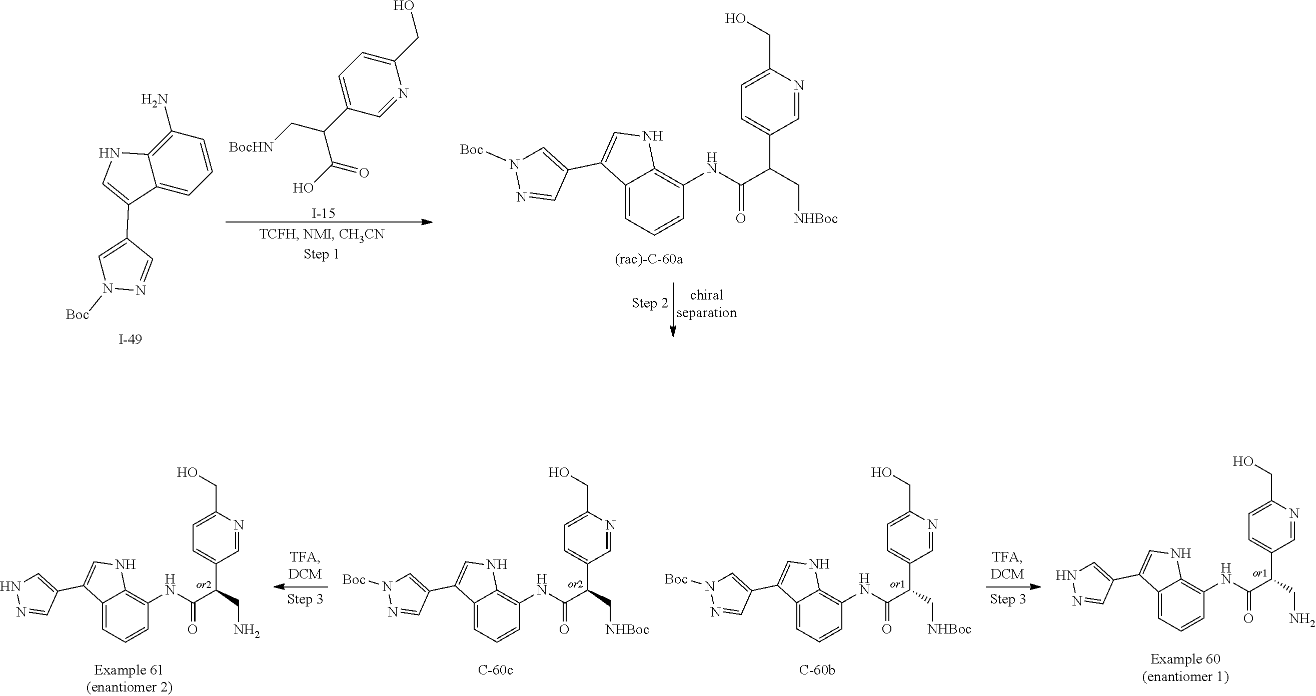

Step 1: Into a 50 mL round-bottom flask were added acid I-15 (297.97 mg, 1.01 mmol, 1 equiv), amine I-49 (300 mg, 1.01 mmol, 1 equiv), TCFH (423.20 mg, 1.51 mmol, 1.5 equiv), NMI (412.81 mg, 5.03 mmol, 5 equiv) and MeCN (10 mL) at room temperature. The resulting mixture was stirred for overnight at room temperature. The residue was purified by reverse flash chromatography with the following conditions: column, C18 silica; mobile phase, MeCN in water (0.5% NH₃ aq), 10% to 50% gradient in 10 min; detector, UV 254 nm to afford racemic amide C-60a (80 mg, 12.51%) as a brown yellow solid. LCMS: m/z (ESI), [M+H]⁺=577.35.

Step 2: The racemic amide C60a (80 mg, 12.51%) was purified by prep-Chiral HPLC with the following conditions (Column: CHIRALPAK IG, 2*25 cm, 5 um; Mobile Phase A: Hex (0.2% DEA), Mobile Phase B: EtOH:DCM (1:1, v/v); Flow rate: 20 mL/min; Gradient: 30% B to 30% B in 26 min; wavelength: 220/254 nm; t_{R}−1=14.51 min; t_{R}−2=20.12 min; Sample Solvent: EtOH:DCM (1:1, v/v); Injection Volume: 0.7 mL) to afford enantiomer C-60c (28 mg 35%), SFC: t_{R}=3.614 min, ee=100%, and enantiomer C-60b (29 mg, 36.25%) as brown yellow solid, SFC: t_{R}=4.986 min, ee=100%.

Step 3: Into a 50 mL round-bottom flask were added enantiomer C₆₀c (30 mg, 0.052 mmol, 1 equiv), TFA (1 mL) and DCM (3 mL) at room temperature. The resulting mixture was stirred for 1 h at room temperature. The mixture was basified to pH 9 with NH₃ aq. The crude product (28 mg) was purified by prep-HPLC with the following conditions (Column: XBridge Prep OBD C18 30*150 mm, 5 um; Mobile Phase A: water (10 mmol/L NH₄HCO₃), Mobile Phase B: ACN; Flow rate: 60 mL/min; Gradient: 2% B to 25% B in 8 min; wavelength: 254 nm; t_{R}=7.23 min) to afford Example 61, enantiomer 2 (10 mg, 50.40%) as a white solid. LCMS: m/z (ESI), [M+H]⁺=377.2. ¹H NMR (CD₃OD, 400 MHZ) δ 3.06 (1H, m), 3.47 (1H, m), 4.00 (1H, m), 4.73 (2H, s), 7.01-7.10 (2H, m), 7.48 (1H, s), 7.56-7.73 (2H, m), 7.92 (2H, s), 8.00 (1H, m), 8.60 (1H, d). SFC: t_{R}=4.285 min, ee=98.7%.

Into a 50 mL round-bottom flask were added enantiomer C-60b (30 mg, 0.052 mmol, 1 equiv), TFA (1 mL) and DCM (3 mL) at room temperature. The resulting mixture was stirred for 1 h at room temperature. The residue was basified to pH 9 with NH₃ aq. The crude product (29 mg) was purified by prep-HPLC with th following conditions (Column: XBridge Shield RP18 OBD 30*150 mm, 5 um; Mobile Phase A: water (10 mmol/L NH₄HCO₃), Mobile Phase B: ACN; Flow rate: 60 mL/min; Gradient: 3% B to 29% B in 8 min; wavelength: 220 nm; t_{R}=7.80 min) to afford Example 60, enantiomer 1 (7.8 mg, 39.72%) as a white solid. LCMS: m/z (ESI), [M+H]⁺=377.2. ¹H NMR (CD₃OD, 400 MHZ) δ 3.06 (1H, m), 3.47 (1H, m), 4.00 (1H, m), 4.73 (2H, s), 7.01-7.10 (2H, m), 7.48 (1H, s), 7.56-7.73 (2H, m), 7.92 (2H, s), 8.00 (1H, m), 8.60 (1H, d). SFC: t_{R}=3.815 min, ee=95.34%.

### Example 62 (Enantiomer 1) and Example 63 (Enantiomer 2)

### Preparation of N-(3-(1H-pyrazol-4-yl)-1H-indol-7-yl)-3-amino-2-(5-methylpyridin-3-yl)propanamide

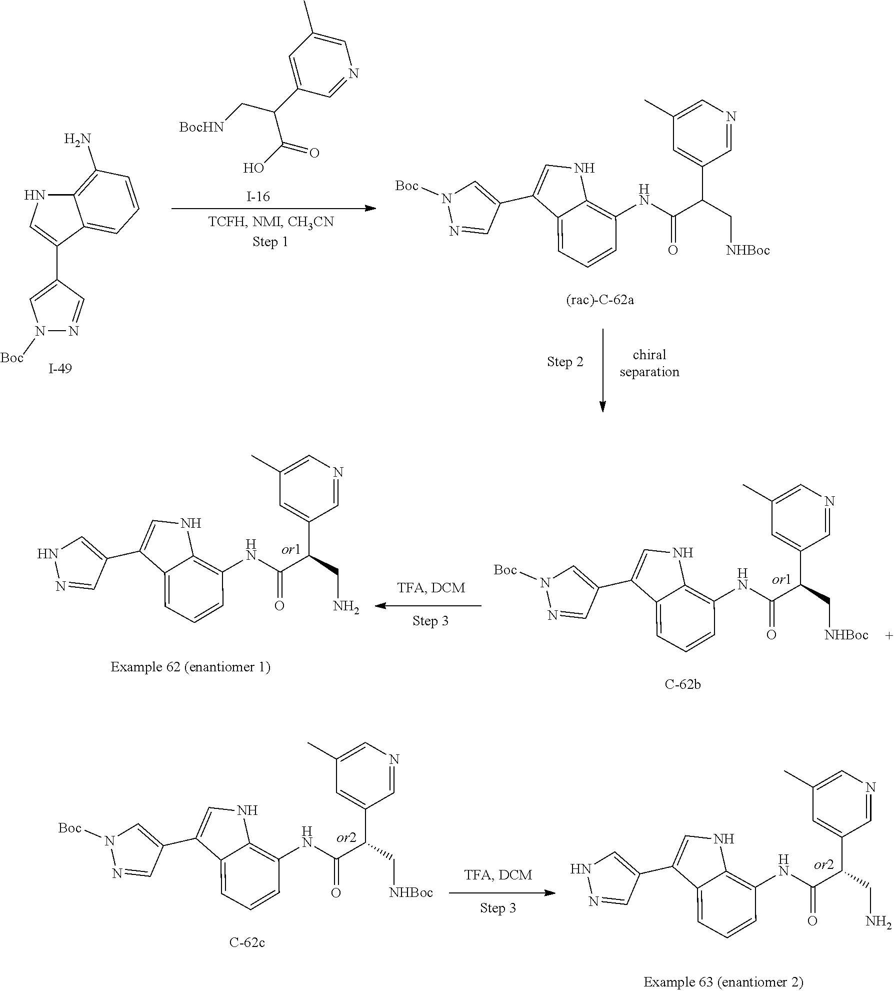

Step 1: A solution of acid I-16 (124.03 mg, 0.44 mmol, 1.2 equiv) and amine I-49 (110 mg, 0.37 mmol, 1.00 equiv), TCFH (155.17 mg, 0.55 mmol, 1.5 equiv), NMI (105.95 mg, 1.29 mmol, 3.5 equiv) in CH₃CN (3 mL) was stirred for 1 h at room temperature under nitrogen atmosphere. The residue was purified by prep-TLC with CH₂Cl₂/methanol (10:1, v/v) to afford racemic amide C-62a (287 mg, 90.46%) as a light yellow solid. LCMS: m/z (ESI), [M+H]⁺=561.35.

Step 2: The racemic amide C-62a (287 mg) was purified by prep-Chiral HPLC with the following conditions (Column: CHIRALPAK IC, 2*25 cm, 5 um; Mobile Phase A: Hex (0.2% DEA), Mobile Phase B: EtOH:DCM (1:1, v/v); Flow rate: 20 mL/min; Gradient: 40% B to 40% B in 13 min; wavelength: 220/254 nm; t_{R}−1=7.14 min; t_{R}−2=10.33 min; Sample Solvent: EtOH:DCM (1:1, v/v); Injection Volume: 0.6 mL) to afford enantiomer C-62b (100 mg, 34.84%), SFC: t_{R}=1.875 min, ee=100%, and enantiomer C-62c (80 mg, 27.87%) as a light yellow solid, SFC: t_{R}=2.41 min, ee=100%.

Step 3: A solution of enantiomer C-62c (80 mg, 0.14 mmol, 1 equiv) and TFA (0.8 mL) in DCM (2 mL) was stirred for 30 min at room temperature under nitrogen atmosphere. The resulting mixture was concentrated under reduced pressure. The residue was dissolved in methanol (2 mL). The mixture was basified to pH 9 with NH₃ (aq) at 0° C. The resulting mixture was stirred for 30 min at 0° C. under nitrogen atmosphere. The crude product (40 mg) was purified by prep-HPLC with the following conditions (Column: XBridge Shield RP18 OBD 30*150 mm, 5 um; Mobile Phase A: water (10 mmol/L NH₄HCO₃), Mobile Phase B: ACN; Flow rate: 60 mL/min; Gradient: 10% B to 30% B in 8 min; wavelength: 220 nm; t_{R}=7.78 min) to afford Example 63, enantiomer 2 (14.0 mg, 35.00%) as a white solid. LCMS: m/z (ESI), [M+H]⁺=361.20. ¹H NMR (CD₃OD, 400 MHZ) δ 2.41 (3H, s), 3.11 (1H, dd), 3.50 (1H, dd), 4.05 (1H, dd), 7.02-7.12 (2H, m), 7.47 (1H, s), 7.65 (1H, dd), 7.83 (1H, d), 7.91 (2H, s), 8.37 (1H, d), 8.48 (1H, d). SFC: t_{R}=1.928 min, ee=99.62%.

A solution of enantiomer C-62b (100 mg, 0.18 mmol, 1 equiv) and TFA (0.8 mL) in DCM (2 mL) was stirred for 30 min at room temperature under nitrogen atmosphere. The resulting mixture was concentrated under reduced pressure. The residue was dissolved in methanol (2 mL). The mixture was basified to pH 9 with NH₃ aq at 0° C. The resulting mixture was stirred for 30 min at 0° C. under nitrogen atmosphere. The crude product (40 mg) was purified by prep-HPLC with the following conditions (Column: XBridge Shield RP18 OBD 30*150 mm, 5 um; Mobile Phase A: water (10 mmol/L NH₄HCO₃), Mobile Phase B: ACN; Flow rate: 60 mL/min; Gradient: 7% B to 33% B in 8 min; wavelength: 220 nm; t_{R}=7.73 min) to afford Example 62, enantiomer 1 (22 mg, 55.00%) as a white solid. LCMS: m/z (ESI), [M+H]⁺=361.20. ¹H NMR (CD₃OD, 400 MHZ) δ 2.41 (3H, s), 3.11 (1H, dd), 3.50 (1H, dd), 4.05 (1H, dd), 7.02-7.12 (2H, m), 7.47 (1H, s), 7.65 (1H, dd), 7.83 (1H, d), 7.91 (2H, s), 8.37 (1H, d), 8.48 (1H, d). SFC: t_{R}=0.503 min, ee=100%.

### Example 64 (Enantiomer 1) and Example 65 (Enantiomer 2)

### Preparation of N-(3-(1H-pyrazol-4-yl)-1H-indol-7-yl)-3-amino-2-(3-hydroxyphenyl)propanamide

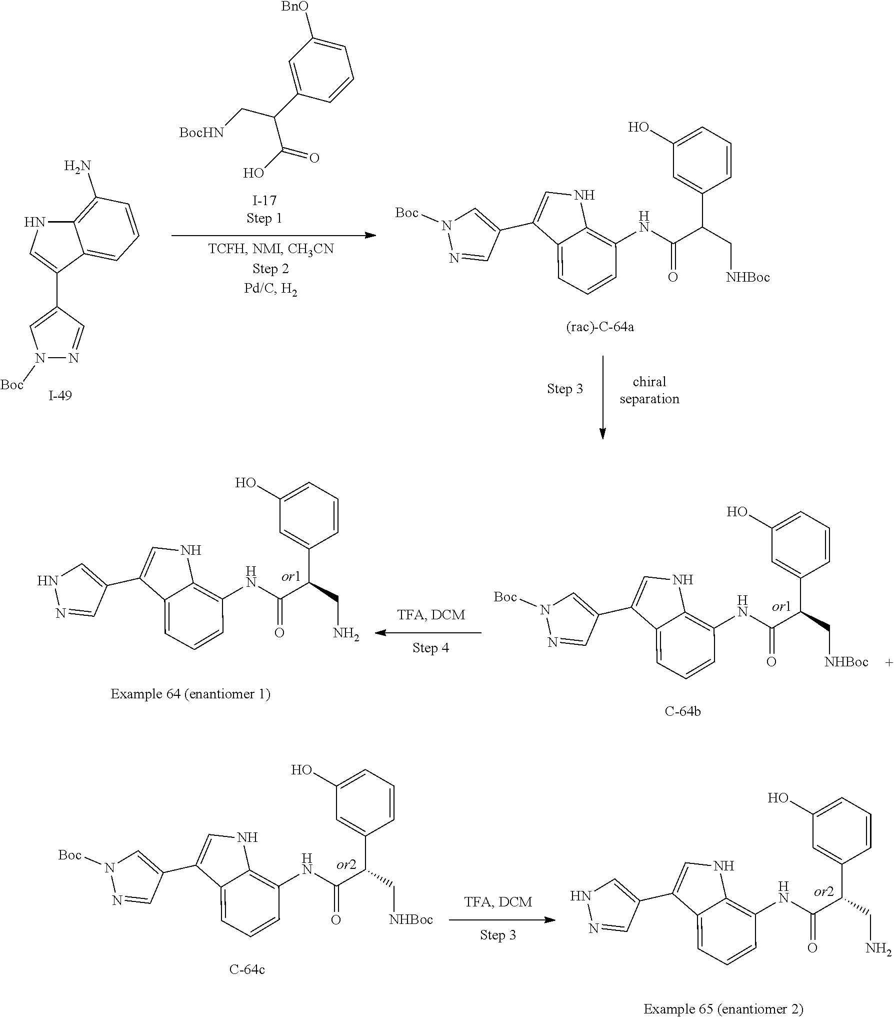

Step 1: Into a 20 mL vial were added acid I-17 (448.19 mg, 1.21 mmol, 1.2 equiv), TCFH (423.20 mg, 1.51 mmol, 1.5 equiv), NMI (330.24 mg, 4.024 mmol, 4.0 equiv) and amine I-49 (300 mg, 1.01 mmol, 1.00 equiv) in CH₃CN (3 mL) at room temperature. The resulting mixture was stirred for 2 h at room temperature under nitrogen atmosphere. The resulting mixture was concentrated under reduced pressure. The residue was purified by prep-TLC with CH₂Cl₂/methanol (40:1, v/v) to afford tert-butyl 4-(7-{2-[3-(benzyloxy)phenyl]-3-[(tert-butoxycarbonyl)amino]propanamido}-1H-indol-3-yl) pyrazole-1-carboxylate (350 mg, 53.40%) as a yellow solid. LCMS: m/z (ESI), [M+H]⁺=652.20. ¹H NMR (400 MHZ, DMSO-d₆) δ 1.37 (9H, s), 1.62 (9H, s), 3.39 (1H, d), 3.55-3.65 (1H, m), 4.07 (1H, t), 5.09 (2H, s), 6.93 (1H, dd), 7.00-7.06 (2H, m), 7.06-7.10 (1H, m), 7.27 (1H, d), 7.32-7.35 (1H, m), 7.35-7.41 (2H, m), 7.42-7.50 (3H, m), 7.61 (1H, d), 7.85 (1H, d), 8.27 (1H, s), 8.49 (1H, s).

Step 2: Into a 50 mL round-bottom flask were added tert-butyl 4-(7-{2-[3-(benzyloxy)phenyl]-3-[(tert-butoxycarbonyl)amino]propanamido}-1H-indol-3-yl) pyrazole-1-carboxylate (330 mg, 0.51 mmol, 1 equiv) and Pd/C (20 mg, 0.19 mmol, 0.37 equiv) in methanol (5 mL) at room temperature. The resulting mixture was stirred for overnight at room temperature under hydrogen atmosphere. The resulting mixture was filtered and the filtered cake was washed with methanol (4×20 mL). The filtrate was concentrated under reduced pressure. The residue was purified by reverse flash chromatography with the following conditions: column, C18 silica; mobile phase, methanol in water, 0% to 100% gradient in 30 min; detector, UV 254 nm. The resulting mixture was concentrated under vacuum to afford racemic amide C-64a (80 mg, 28.13%) as a white solid. LCMS: m/z (ESI), [M+H]⁺=562.20.

Step 3: The racemic amide C-64a (80 mg) was purified by prep-Chiral HPLC with the following conditions (Column: CHIRALPAK IC, 2*25 cm, 5 um; Mobile Phase A: Hex (0.2% DEA), Mobile Phase B: EtOH:DCM (1:1, v/v); Flow rate: 20 mL/min; Gradient: 20% B to 20% B in 21 min; wavelength: 220/254 nm; t_{R}−1=12.04 min; t_{R}−2=16.90 min; Sample Solvent: EtOH:DCM (1:1, v/v); Injection Volume: 0.8 mL) to afford enantiomer C-64b (25 mg, 31.25%), SFC: t_{R}=2.323 min, ee=100%, and enantiomer C-64c (23 mg, 28.75%) as a yellow solid, SFC: t_{R}=3.04 min, ce=98.84%.

Step 4: Into a 50 mL round-bottom flask were added enantiomer C-64b (20 mg, 0.036 mmol, 1 equiv) and TFA (0.5 mL) in DCM (1 mL) at room temperature. The resulting mixture was stirred for 1 h at room temperature. The resulting mixture was concentrated under reduced pressure. The mixture was basified to pH 9 with NH₃ (aq). The crude product was purified by prep-HPLC with the following conditions (Column: XBridge Prep OBD C18 30*150 mm, 5 um; Mobile Phase A: water (10 mmol/L NH₄HCO₃), Mobile Phase B: ACN; Flow rate: 60 mL/min; Gradient: 5% B to 35% B in 7 min; wavelength: 220 nm; t_{R}=7.08 min) to afford Example 64, enantiomer 1 (11.9 mg, 92.47%) as a white solid. LCMS: m/z (ESI), [M+H]⁺=362.05. ¹H NMR (CD₃OD, 400 MHZ) δ 3.03 (1H, dd), 3.41 (1H, dd), 3.89 (1H, dd), 6.74-6.78 (1H, m), 6.93-6.97 (2H, m), 7.03-7.12 (2H, m), 7.22 (1H, dd), 7.47 (1H, s), 7.64 (1H, dd), 7.92 (2H, s). SFC: t_{R}=3.572 min, ee=100.00%.

Into a 25 mL vial were added enantiomer C-64c (30 mg, 0.053 mmol, 1 equiv) and TFA (1.50 mL) in DCM (1 mL) at room temperature. The resulting mixture was stirred for 1 h at room temperature. The resulting mixture was concentrated under reduced pressure. The mixture was basified to pH 9 with NH₃ (aq). The crude product was purified by Prep-HPLC with the following conditions (Column: XBridge Shield RP18 OBD 30*150 mm, 5 um; Mobile Phase A: water (10 mmol/L NH₄HCO₃+0.1% NH₃ aq), Mobile Phase B: ACN; Flow rate: 60 mL/min; Gradient: 5% B to 35% B in 8 min; wavelength: 220 nm; t_{R}=8.30 min) to afford Example 65, enantiomer 2 (12.6 mg, 65.27%) as a white solid. LCMS: m/z (ESI), [M+H]⁺=362.05. ¹H NMR (CD₃OD, 400 MHz) δ 3.04 (1H, dd), 3.41 (1H, dd), 3.90 (1H, dd), 6.74-6.78 (1H, m), 6.93-6.97 (2H, m), 7.03-7.12 (2H, m), 7.22 (1H, t), 7.47 (1H, s), 7.64 (1H, dd), 7.92 (2H, s). SFC: t_{R}=4.347 min, ee=92.08%.

### Example 66 (Enantiomer 1) and Example 67 (Enantiomer 2)

### Preparation of N-(3-(1H-pyrazol-4-yl)-1H-indol-7-yl)-3-(4-methylpiperazin-1-yl)-2-(pyridin-3-yl)propanamide

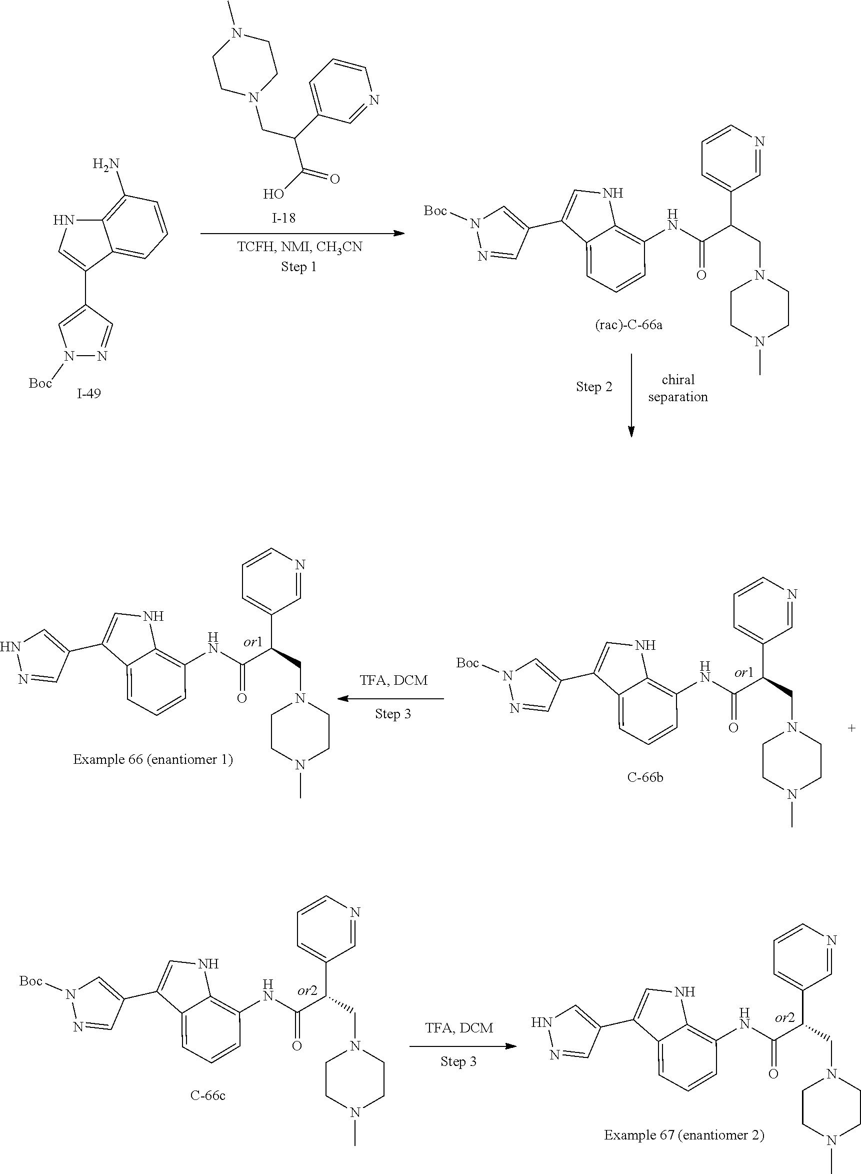

Step 1: To a stirred mixture of acid I-18 (300 mg, 1.20 mmol, 1 equiv) and amine I-49 (359.00 mg, 1.20 mmol, 1.00 equiv) in CH₃CN (30 mL) was added TCFH (675.24 mg, 2.41 mmol, 2 equiv) and NMI (493.99 mg, 6.02 mmol, 5 equiv) at room temperature under nitrogen atmosphere. The mixture was stirred for 2 h at room temperature under nitrogen atmosphere. The residue was purified by reverse flash chromatography with the following conditions: column, C18 silica; mobile phase, MeCN in water, 10% to 50% gradient in 10 min; detector, UV 254 nm. This resulted in racemic amide C-66a (151 mg, 23.69%) as a brown solid. LCMS: m/z (ESI), [M+H]⁺=530.35.

Step 2: The racemic amide C-66a (205 mg) was purified by prep-Chiral HPLC with the following conditions (Column: CHIRALPAK IC, 2*25 cm, 5 um; Mobile Phase A: Hex (0.2% DEA), Mobile Phase B: EtOH:DCM (1:1, v/v); Flow rate: 20 mL/min; Gradient: 40% B to 40% B in 14.5 min; wavelength: 220/254 nm; t_{R}−1=8.02 min; t_{R}−2=11.66 min; Sample Solvent: EtOH:DCM (1:1, v/v); Injection Volume: 0.8 mL) to afford enantiomer C-66c as a yellow solid, SFC: t_{R}=2.186, ee=100%, and enantiomer C-66b as yellow solid, SFC: t_{R}=3.308, ee=100%.

To a stirred mixture of enantiomer C-66b (80 mg, 0.15 mmol, 1 equiv) in DCM (6 mL) was added TFA (1.5 mL) at room temperature under nitrogen atmosphere. The mixture was stirred for 1.5 h at room temperature under nitrogen atmosphere. The reaction mixture was basified to pH 8 with NH₃ (aq). The mixture was purified by prep-HPLC with the following conditions (Column: XBridge Shield RP18 OBD 30*150 mm, 5 um; Mobile Phase A: water (10 mmol/L NH₄HCO₃+0.1% NH₃ aq), Mobile Phase B: ACN; Flow rate: 60 mL/min; Gradient: 13% B to 35% B in 8 min; wavelength: 220 nm; t_{R}=7.68 min) to afford Example 66, enantiomer 1 (37.8 mg, 58.26%) as a white solid. LCMS m/z (ESI), [M+H]⁺=430.20. ¹H NMR (CD₃OD, 400 MHZ) δ 2.31 (3H, s), 2.51-2.91 (9H, m), 3.35-3.4 (1H, m), 4.21 (1H, dd), 7.06-7.12 (2H, m), 7.45-7.53 (2H, m), 7.68 (1H, dd), 8.05 (3H, d), 8.50 (1H, dd), 8.66 (1H, d). SFC: t_{R}=1.958, ee=100%.

To a stirred mixture of enantiomer C-66c (80 mg, 0.15 mmol, 1 equiv) in DCM (6 mL) was added TFA (1.5 mL) at room temperature under nitrogen atmosphere. The mixture was stirred for 1.5 h at room temperature under nitrogen atmosphere. The reaction mixture was basified to pH 8 with NH₃ aq The mixture was purified by prep-HPLC with the following conditions (Column: XBridge Shield RP18 OBD 30*150 mm, 5 um; Mobile Phase A: water (10 mmol/L NH₄HCO₃+0.1% NH₃ aq), Mobile Phase B: ACN; Flow rate: 60 mL/min; Gradient: 13% B to 35% B in 8 min; wavelength: 220 nm; t_{R}=7.68 min) to afford Example 67, enantiomer 2 (37.8 mg, 58.26%) as a white solid. LCMS: m/z (ESI), [M+H]⁺=430.25. ¹H NMR (CD₃OD, 400 MHZ) δ 2.31 (3H, s), 2.46-2.90 (9H, m), 3.37-3.44 (1H, m), 4.21 (1H, dd), 7.05-7.14 (2H, m), 7.44-7.54 (2H, m), 7.68 (1H, dd), 7.93 (2H, d), 8.02-8.07 (1H, m), 8.50 (1H, dd), 8.66 (1H, d). SFC: t_{R}=2.053 min, ce=100%.

### Example 68 (Enantiomer 1), Example 69 (Enantiomer 2), and Example 100 (Racemic)

### Preparation of N-(3-(1H-pyrazol-4-yl)-1H-indol-7-yl)-2-amino-2-(3-cyanophenyl) acetamide

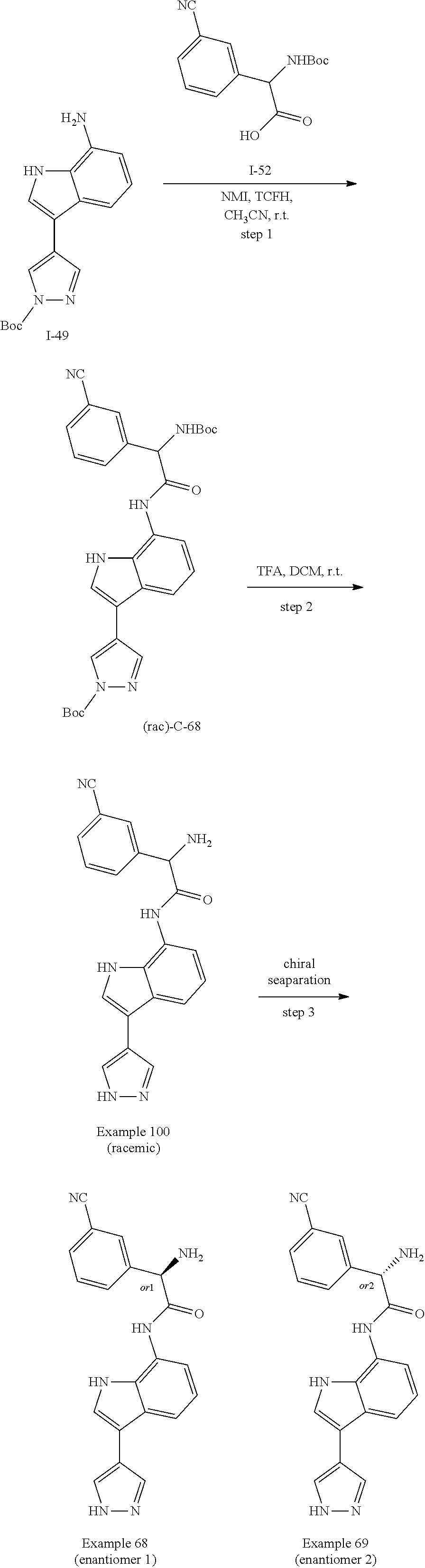

Step 1: A mixture of acid I-52 (300 mg, 1.09 mmol, 1 equiv) in CH₃CN (6.0 mL) was added NMI (267.45 mg, 3.26 mmol, 3 equiv), TCFH (609.31 mg, 2.17 mmol, 2 equiv) and amine I-49 (327.19 mg, 1.10 mmol, 1.01 equiv). The resulting mixture was stirred for 2 h at 25° C. under nitrogen atmosphere. The resulting mixture was diluted with water (15 mL). The resulting mixture was extracted with ethyl acetate (3×20 mL). The combined organic layers were dried over anhydrous Na₂SO₄. After filtration, the filtrate was concentrated under reduced pressure. The residue was purified by prep-TLC with CH₂Cl₂/methanol (20:1, v/v) to afford racemic amide C-68 (510 mg, 84.38%) as a yellow oil. LCMS: m/z (ESI), [M-Boc+H]⁺=457.15.

Step 2: A mixture of racemic amide C-68 (320 mg, 0.58 mmol, 1 equiv) in TFA (1.0 mL) and DCM (4.0 mL) was stirred for 1.0 h at room temperature under nitrogen atmosphere. After reaction, the resulting mixture was concentrated under vacuum and diluted with methanol (2.0 mL), the residue was basified to pH 9 with NH₃ aq. The crude product was purified by prep-HPLC with the following conditions (Column: XBridge Prep OBD C18 30*150 mm, 5 um; Mobile Phase A: water (10 mmol/L NH₄HCO₃), Mobile Phase B: ACN; Flow rate: 60 mL/min; Gradient: 15% B to 40% B in 8 min; wavelength: 254 nm; t_{R}=7.05 min) to afford Example 100, racemic (200 mg, 97.62%) as a white solid.

Step 3: Example 100, racemic (200 mg) was purified by chiral HPLC with the following conditions (Column: CHIRALPAK IG, 2*25 cm, 5 um; Mobile Phase A: Hex (0.2% DEA), Mobile Phase B: EtOH/DCM (1:1, v/v); Flow rate: 20 mL/min; Gradient: 50% B to 50% B in 8 min; wavelength: 220/254 nm; t_{R}−1=4.12 min; t_{R}−2=5.81 min; Sample Solvent: EtOH/DCM (1:1, v/v); Injection Volume: 0.3 mL) to afford Example 68, enantiomer 1 (11.7 mg, 22.08%) as a white solid after prep-HPLC (Column: XBridge Prep OBD C18 30*150 mm, 5 um; Mobile Phase A: water (10 mmol/L NH₄HCO₃), Mobile Phase B: ACN; Flow rate: 60 mL/min; Gradient: 15% B to 40% B in 8 min, 40% B; wavelength: 254 nm; t_{R}=7.05 min). LCMS: m/z (ESI), [M+H]⁺=357.15. ¹H NMR (CD₃OD, 400 MHZ) δ 4.82 (1H, s), 7.12 (2H, m), 7.47 (1H, s), 7.64 (2H, m), 7.73 (1H, d), 7.91 (1H, d), 7.98 (1H, s), SFC: t_{R}=5.974 min, ee=93.35%, and Example 69, enantiomer 2 (3.7 mg, 4.63%) as a white solid after prep-HPLC (Column: Xselect CSH F-Phenyl OBD column, 19*250 mm, 5 um; Mobile Phase A: water (10 mmol/L NH₄HCO₃), Mobile Phase B: methanol; Flow rate: 25 mL/min; Gradient: 38% B to 57% B in 11 min; wavelength: 254 nm; t_{R}=10.61 min). LCMS: m/z (ESI), [M+H]=357.15. ¹H NMR (CD₃OD, 400 MHZ) δ 4.82 (1H, s), 7.11 (2H, m), 7.64 (4H, m), 7.94 (4H, m). SFC: t_{R}=6.817, ce=81.22%.

### Example 70 (Racemic)

### Preparation of 3-(2-amino-1-{[3-(1H-pyrazol-4-yl)-1H-indol-7-yl]carbamoyl}ethyl)benzoic acid

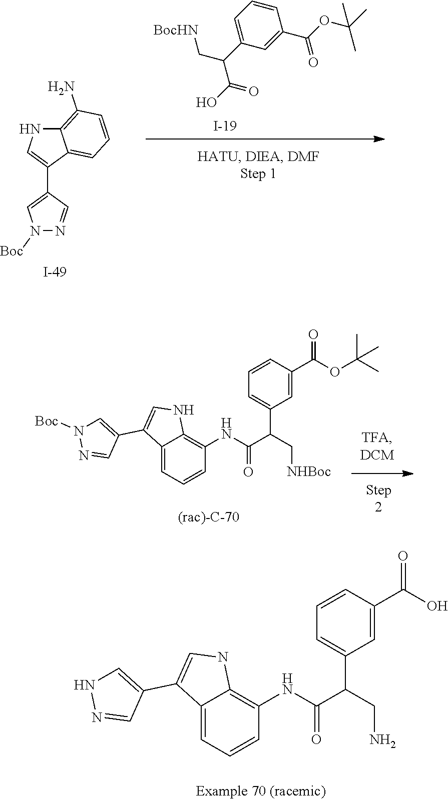

Step 1: Into a 8 mL vial were added acid I-19 (122.48 mg, 0.34 mmol, 1 equiv) and amine I-49 (100 mg, 0.34 mmol, 1 equiv), HATU (191.17 mg, 0.50 mmol, 1.5 equiv) in DMF (2 mL), DIEA (129.96 mg, 1.01 mmol, 3 equiv) was added to the above mixture at 0° C. The resulting mixture was stirred for 1 h at room temperature under nitrogen atmosphere. The resulting mixture was diluted with ethyl acetate (40 mL). The resulting mixture was washed with NaHCO₃ (1×40 mL), water (1×40 mL) and saturated brine (2×40 mL). The organic layer was dried over anhydrous Na₂SO₄. After filtration, the filtrate was concentrated under reduced pressure. The residue was purified by prep-TLC with CH₂Cl₂/methanol (30:1, v/v) to afford racemic amide C-70 (150 mg, 84.88%) as a off-white solid. LCMS: m/z (ESI), [M+H]⁺=646.45.

Step 2. Into a 50 mL round-bottom flask were added racemic amide C-70 (60 mg, 0.093 mmol, 1 equiv) in DCM (3 mL) and TFA (1 mL) at room temperature. The resulting mixture was stirred for 1 h at room temperature under nitrogen atmosphere. The mixture was concentrated under reduced pressure and dissolved in DMF (1 mL), basified to pH 9 with NH₃ (aq). The crude product (50 mg) was purified by prep-HPLC with the following conditions (Column: Xselect CSH C18 OBD 30*150 mm 5 um, n; Mobile Phase A: ACN, Mobile Phase B: ACN; Flow rate: 25 mL/min; Gradient: 61% B to 83% B in 8 min; wavelength: 254 nm; t_{R}=9.2 min) to afford Example 70, racemic (25.9 mg, 71.58%) as a white solid. LCMS: m/z (ESI), [M+H]⁺=390.15. ¹H NMR (DMSO-d₆, 400 MHz) δ 2.97 (1H, dd), 3.39 (1H, dd), 4.32 (1H, dd), 6.99 (1H, t), 7.38 (1H, t), 7.44-7.61 (3H, m), 7.69 (1H, d), 7.77-8.10 (3H, m), 8.22 (1H, d).

### Example 71 (Racemic)

### Preparation of 4-(2-amino-1-{[3-(1H-pyrazol-4-yl)-1H-indol-7-yl]carbamoyl}ethyl)benzoic acid

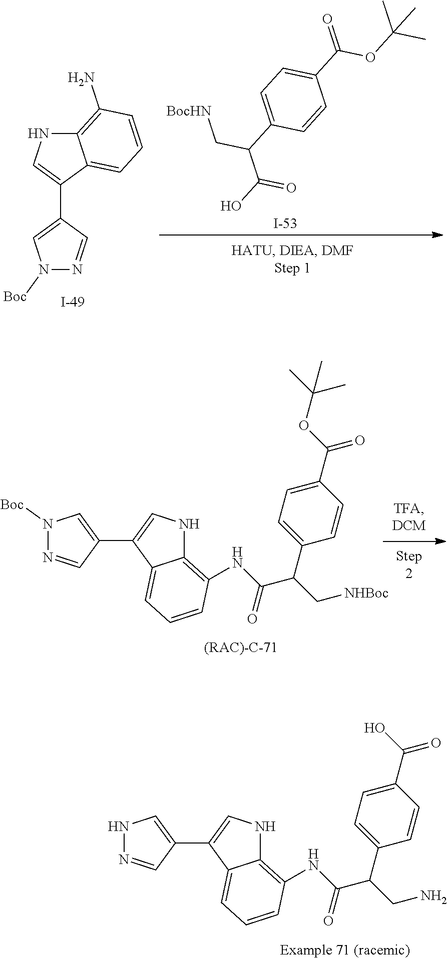

Step 1: To a stirred solution of acid I-53 (50 mg, 0.14 mmol, 1 equiv) and amine I-49 (48.99 mg, 0.16 mmol, 1.2 equiv) in DMF (4 mL) were added HATU (104.05 mg, 0.27 mmol, 2 equiv) and DIEA (53.05 mg, 0.411 mmol, 3 equiv) at room temperature under air atmosphere. The resulting mixture was stirred for 3 h at room temperature under air atmosphere. The reaction was quenched by the addition of water (5 mL) at room temperature. The resulting mixture was extracted with ethyl acetate (3×10 mL). The combined organic layers were washed with brine (3×10 mL), dried over anhydrous Na₂SO₄. After filtration, the filtrate was concentrated under reduced pressure. The residue was purified by pPrep-TLC with CH₂Cl₂/methanol (10:1, v/v) to afford racemic amide C-71 (50 mg, 56.59%) as a yellow oil. LCMS: m/z (ESI), [M+H]⁺=646.40.

Step 2: To a stirred solution of racemic amide C-71 (150 mg, 0.23 mmol, 1 equiv) in DCM (5 mL) was added TFA (1 mL) at room temperature under air atmosphere. The resulting mixture was stirred for 2 h at room temperature under air atmosphere. The reaction was concentrated under reduced pressure. The mixture was dissolved with DMF (1 mL), basified to pH 8 with NH₃ aq. The crude product was purified by prep-HPLC with the following conditions (Column: XBridge Shield RP18 OBD 30*150 mm, 5 um; Mobile Phase A: water (10 mmol/L NH₄HCO₃+0.1% NH₃ aq), Mobile Phase B: ACN; Flow rate: 60 mL/min; Gradient: 22% B to 43% B in 8 min; wavelength: 220 nm; t_{R}=7.80 min) to afford Example 71, racemic (15.7 mg, 17.36%) as a white solid. LCMS: m/z (ESI), [M+H]⁺=390.10. ¹H NMR (DMSO-d₆, 400 MHz) δ 2.99 (1H, dd), 3.20-3.25 (3H, m), 4.07-4.15 (1H, m), 6.99 (1H, t), 7.51 (3H, dd), 7.55-7.61 (2H, m), 7.95 (4H, d), 11.22 (1H, s).

### Example 72 (Racemic)

### Preparation of 4-(2-amino-1-{[3-(1H-pyrazol-4-yl)-1H-indol-7-yl]carbamoyl}ethyl)-N,N-dimethylbenzamide

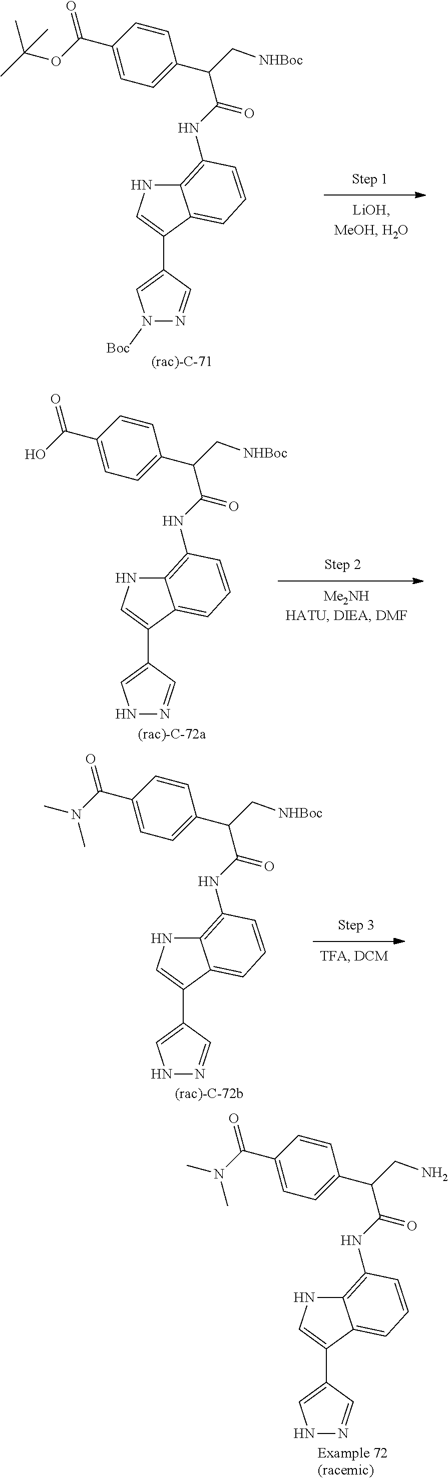 [SMILES: CC(C)(C)OC(=O)NCC(C(=O)Nc1cccc2c(-c3cnn(C(=O)OC(C)(C)C)c3)c[nH]c12)c1ccc(C(=O)OC(C)(C)C)cc1 ; CC(C)(C)OC(=O)NCC(C(=O)Nc1cccc2c(-c3cn[nH]c3)c[nH]c12)c1ccc(C(=O)O)cc1 ; CN(C)C(=O)c1ccc(C(CNC(=O)OC(C)(C)C)C(=O)Nc2cccc3c(-c4cn[nH]c4)c[nH]c23)cc1 ; CN(C)C(=O)c1ccc(C(CN)C(=O)Nc2cccc3c(-c4cn[nH]c4)c[nH]c23)cc1]

Step 1: To a stirred solution of racemic amide C-71 (350 mg, 0.54 mmol, 1 equiv) in methanol (20 mL), H₂O (5 mL) were added LiOH (51.92 mg, 2.17 mmol, 4 equiv) at room temperature. The mixture was stirred for 2 h at room temperature under air atmosphere. The resulting mixture was concentrated under reduced pressure. The residue was purified by reverse flash chromatography with the following conditions: column, C18 silica; mobile phase, MeCN in water, 50% to 60% gradient in 10 min; detector, UV 254 nm to afford racemic acid C-72a (300 mg, 113.07%) as a pink solid. LCMS: m/z (ESI), [M+H]⁺=490.53.

Step 2: To a stirred solution of racemic acid C-72a (120 mg, 0.25 mmol, 1 equiv) and dimethylamine (13.26 mg, 0.29 mmol, 1.2 equiv) in DMF (8 mL, 103.37 mmol) were added HATU (186.42 mg, 0.49 mmol, 2 equiv) and DIEA (95.05 mg, 0.74 mmol, 3 equiv) at room temperature under air atmosphere. The resulting mixture was stirred for 3 h at room temperature under air atmosphere. The reaction was quenched by the addition of water (10 mL) at room temperature. The resulting mixture was extracted with ethyl acetate (3×10 mL). The combined organic layers were washed with brine (3×10 mL), dried over anhydrous Na₂SO₄. After filtration, the filtrate was concentrated under reduced pressure. The resulting mixture was concentrated under reduced pressure. The residue was purified by prep-TLC with PE/EA (1:1, v/v) to afford racemic amide C-72b (90 mg, 71.07%) as a white solid. LCMS: m/z (ESI), [M+H]⁺=517.60.

Step 3: To a stirred solution of racemic amide C-72b (70 mg, 0.11 mmol, 1 equiv) in DCM (5 mL, 78.65 mmol) was added TFA (1 mL, 13.46 mmol) at room temperature under air atmosphere. The resulting mixture was stirred for 2 h at room temperature under air atmosphere. The reaction was quenched by the addition of water (5 mL) at room temperature. The resulting mixture was extracted with CH₂Cl₂ (3×5 mL). The combined organic layers were dried over anhydrous Na₂SO₄. After filtration, the filtrate was concentrated under reduced pressure. The residue was basified to pH 8 with NH₃ (aq). The crude product was purified by prep-HPLC with the following conditions (Column: Xselect CSH C18 OBD 30*150 mm 5 um; Mobile Phase A: water (0.1% FA), Mobile Phase B: ACN; Flow rate: 60 mL/min; Gradient: 5% B to 20% B in 7 min; wavelength: 254; 220 nm; t_{R}=6.5 min) to afford Example 72, racemic (2 mg, 4.44%) as a white solid. LCMS: m/z (ESI), [M+H]⁺=417.25. ¹H NMR (CD₃OD, 400 MHZ) δ 3.03 (3H, s), 3.13 (3H, s), 3.41 (1H, s), 3.66-3.73 (1H, m), 4.31 (1H, dd), 7.04-7.10 (2H, m), 7.48 (1H, s), 7.54 (2H, d), 7.60-7.70 (3H, m), 7.92 (2H, s).

### Example 73 (Racemic)

### Preparation of 4-(2-amino-1-{[3-(1H-pyrazol-4-yl)-1H-indol-7-yl]carbamoyl}ethyl)-N-methylbenzamide

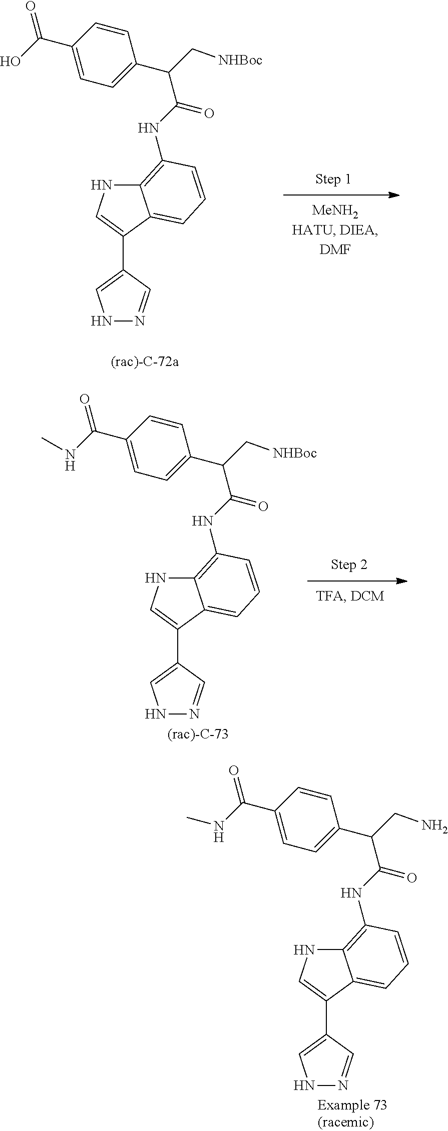 [SMILES: CC(C)(C)OC(=O)NCC(C(=O)Nc1cccc2c(-c3cn[nH]c3)c[nH]c12)c1ccc(C(=O)O)cc1 ; CNC(=O)c1ccc(C(CNC(=O)OC(C)(C)C)C(=O)Nc2cccc3c(-c4cn[nH]c4)c[nH]c23)cc1 ; CNC(=O)c1ccc(C(CN)C(=O)Nc2cccc3c(-c4cn[nH]c4)c[nH]c23)cc1]

Step 1: To a stirred solution of racemic acid C-72a (120 mg, 0.25 mmol, 1 equiv) and methylamine (9.14 mg, 0.29 mmol, 1.2 equiv) in DMF (8 mL, 103.37 mmol) were added HATU (186.42 mg, 0.49 mmol, 2 equiv) and DIEA (95.05 mg, 0.74 mmol, 3 equiv) at room temperature under air atmosphere. The resulting mixture was stirred for 2 h at room temperature under air atmosphere. The reaction was quenched by the addition of water (10 mL) at room temperature. The resulting mixture was extracted with ethyl acetate (3×10 mL). The combined organic layers were washed with brine (3×10 mL), dried over anhydrous Na₂SO₄. After filtration, the filtrate was concentrated under reduced pressure. The residue was purified by prep-TLC with CH₂Cl₂/methanol (10:1, v/v) to afford racemic amide C-73 (50 mg, 40.59%) as a white solid. LCMS: m/z (ESI), [M+H]⁺=503.45.

Step 2: A solution of racemic amide C-73 (50 mg, 0.099 mmol, 1 equiv) in HCl (gas) in 1,4-dioxane (4 mL) was stirred for 2 h at room temperature under air atmosphere. The resulting mixture was concentrated under reduced pressure. The residue was basified to pH 8 with NH₃ (aq). The crude product was purified by prep-HPLC with the following conditions (Column: Xselect CSH C18 OBD 30*150 mm 5 um; Mobile Phase A: water (10 mmol/L NH₄HCO₃), Mobile Phase B: methanol; Flow rate: 25 mL/min; Gradient: 41% B to 56% B in 8 min; wavelength: 254 nm; t_{R}=6.6 min) to afford Example 73, racemic (1.3 mg, 3.25%) as a white solid. LCMS: m/z (ESI), [M+H]⁺=403.45. ¹H NMR (DMSO-d₆, 400 MHz) δ 2.78 (3H, d), 3.17 (1H, d), 4.10 (1H, d), 4.12 (1H, d), 7.00 (1H, t), 7.34 (1H, d), 7.53 (2H, d), 7.57-7.64 (2H, m), 7.85 (2H, d), 7.96 (1H, s), 8.42 (1H, d), 10.69 (1H, s).

### Example 74 (Racemic)

### Preparation of 4-(1-((3-(1H-pyrazol-4-yl)-1H-indol-7-yl)amino)-3-amino-1-oxopropan-2-yl)benzamide

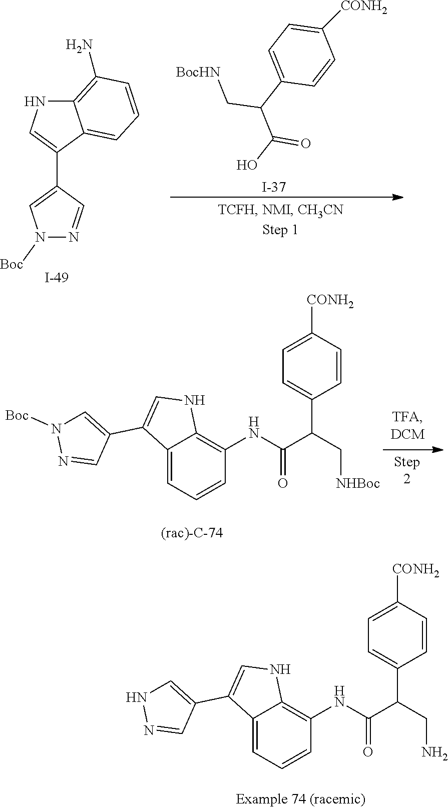

Step 1: Into a 40 mL vial were added acid I-37 (148.82 mg, 0.48 mmol, 1.2 equiv) and amine I-49 (120 mg, 0.40 mmol, 1 equiv) and TCFH (135.43 mg, 0.48 mmol, 1.2 equiv) and NMI (132.10 mg, 1.61 mmol, 4 equiv) and MeCN (8 mL) at room temperature. The resulting mixture was stirred for 1 h at room temperature under nitrogen atmosphere. The resulting mixture was concentrated under reduced pressure. The residue was purified by prep-TLC with CH₂Cl₂/methanol (18:1, v/v) to afford racemic amide C-74 (220 mg, 92.92%) as a brown solid. LCMS: m/z (ESI), [M+H]⁺=589.30.

Step 2: Into a 8 mL vial were added racemic amide C-74 (220 mg, 0.37 mmol, 1 equiv) and TFA (1 mL) and DCM (3 mL) at room temperature. The resulting mixture was stirred for 1 h at room temperature under air atmosphere. The resulting mixture was concentrated under reduced pressure. The residue was dissolved in DMF (2 mL). The mixture was basified to pH 9 with NH₃—H₂O. The resulting mixture was stirred for 30 min at 0° C. under air atmosphere. The crude product (150 mg) was purified by Prep-HPLC with the following conditions (Column: XBridge Prep OBD C18 30*150 mm, 5 um; Mobile Phase A: water (10 mmol/L NH₄HCO₃), Mobile Phase B: ACN; Flow rate: 60 mL/min; Gradient: 5% B to 22% B in 9 min; Wavelength: 254 nm; t_{R}=8.32 min) to afford Example 74, racemic (43.1 mg, 29.65%) as a white solid. LCMS: m/z (ESI), [M+H]⁺=389.15. ¹H NMR (DMSO-d₆, 400 MHZ) δ 2.94 (1H, m), 3.64 (1H, s), 3.95 (1H, s), 6.99 (1H, m), 7.33 (1H, s), 7.40 (1H, m), 7.50 (2H, d), 7.58 (2H, m), 7.85 (2H, d), 7.93 (3H, s).

### Example 75 (Racemic)

### Preparation of 3-amino-2-[3-(hydroxymethyl)phenyl]-N-[3-(1H-pyrazol-4-yl)-1H-indol-7-yl]propanamide

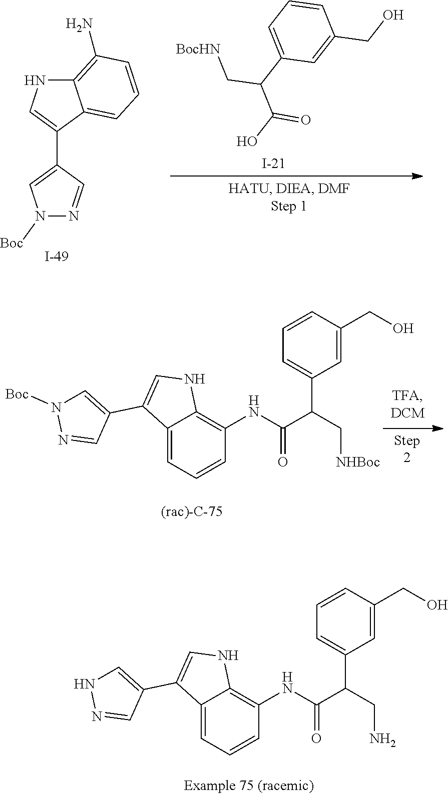

Step 1: Into a 50 mL round-bottom flask were added acid I-21 (100 mg, 0.34 mmol, 1 equiv), amine I-49 (151.53 mg, 0.51 mmol, 1.5 equiv), HATU (193.12 mg, 0.51 mmol, 1.5 equiv), triethylamine (171.32 mg, 1.70 mmol, 5 equiv) in DMF (5 mL) at room temperature. The resulting mixture was stirred for 8 h at room temperature. The resulting mixture was diluted with ethyl acetate (30 mL). The residue was washed with water (3×30 mL). The residue was purified by prep-TLC with CH₂Cl₂/methanol (20:1, v/v) to afford racemic amide C-75 (120 mg, 54.08%) as a yellow oil. LCMS: m/z (ESI), [M+H]⁺=576.25.

Step 2: Into a 50 mL round-bottom flask were added racemic amide C-75 (100 mg, 0.17 mmol, 1 equiv), TFA (1 mL, 13.46 mmol, 77.50 equiv) and DCM (3 mL) at room temperature. The resulting mixture was stirred for 1 h at room temperature. The resulting mixture was concentrated under reduced pressure. The resulting mixture was diluted with methanol (10 mL). The mixture was neutralized to pH 9 with NH₃ (aq). and the resulting mixture was stirred at 0° C. for 30 min. The crude product was purified by prep-HPLC with the following conditions (Column: Xselect CSH C18 OBD 30*150 mm 5 um; Mobile Phase A: water (10 mmol/L NH₄HCO₃), Mobile Phase B: ACN; Flow rate: 60 mL/min; Gradient: 20% B to 35% B in 8 min; wavelength: 254 nm; t_{R}=7.3 min) to afford Example 75, racemic (26 mg, 39.69%) as a white solid. LCMS: m/z (ESI), [M+H]⁺=376.15. ¹H NMR (400 MHZ, CD₃OD) δ 3.06 (dd, 1H), 3.45 (dd, 1H), 3.98 (dd, 1H), 4.65 (s, 2H), 7.07 (m, 2H), 7.33 (d, 1H), 7.40 (m, 2H), 7.49 (d, 2H), 7.64 (dd, 1H), 7.91 (s, 2H).

### Example 76 (Racemic)

### Preparation of 3-amino-2-[3-(aminomethyl)phenyl]-N-[3-(1H-pyrazol-4-yl)-1H-indol-7-yl]propanamide

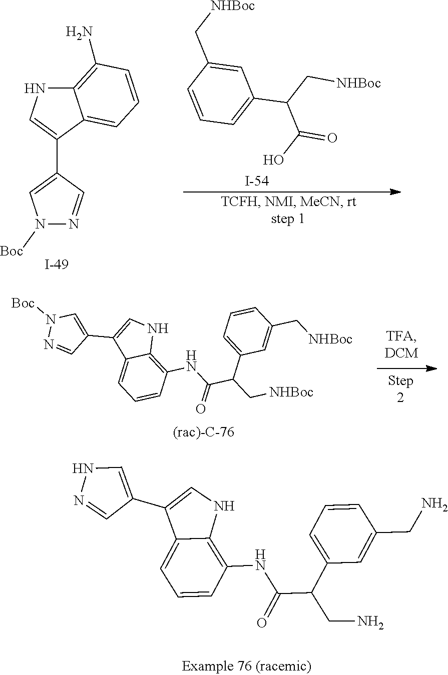

Step 1: To a solution of acid I-54 (126.93 mg, 0.32 mmol, 1.2 equiv) and amine I-49 (80 mg, 0.27 mmol, 1 equiv) in ACN (8 mL) were added TCFH (90.28 mg, 0.32 mmol, 1.2 equiv) and NMI (88.06 mg, 1.07 mmol, 4 equiv) at 25° C. under N₂ atmosphere. The mixture was stirred for 2 hours at 25° C. The resulting mixture was filtered and the filtered cake was washed with methanol (3×100 mL). The filtrate was concentrated under reduced pressure. The residue was purified by prep-TLC with DCM/methanol (10:1, v/v) to afford racemic amide C-76 (145 mg, 65.15%) as a brown solid. LCMS: m/z (ESI), [M+H]⁺=675.50.

Step 2: To a solution of racemic amide C-76 (180 mg, 0.27 mmol, 1 equiv) in DCM (15 mL) was added TFA (5 mL) at 25° C. under N₂ atmosphere. The mixture was stirred for 2 hours at 25° C. The resulting mixture was concentrated under reduced pressure. The residue was dissolved in water (50 mL). The mixture was basified to pH=8 with NaHCO₃. The resulting mixture was extracted with EA (3×50 mL). The aqueous layer was concentrated under vacuum. The crude product (100 mg) was purified by prep-HPLC with the following conditions (Column: YMC-Actus Triart C18 ExRS, 30*150 mm, 5 um; Mobile Phase A: water (50 mmol/L NH₄HCO₃), Mobile Phase B: ACN; Flow rate: 60 mL/min; Gradient: 5% B to 30% B in 7 min; wavelength: 220 nm; t_{R}=6.05 min) to afford Example 76, racemic (19 mg, 18.87%) as a white solid. LCMS: m/z (ESI), [M+H]⁺=375.25. ¹H NMR (CD₃OD, 400 MHz) δ 3.06 (1H, dd), 3.46 (1H, dd), 3.93 (2H, s), 3.99 (1H, m), 7.07 (2H, m), 7.35 (1H, dt), 7.43 (2H, m), 7.46 (1H, d), 7.50 (1H, d), 7.64 (1H, dd), 7.91 (2H, s).

### Example 77 (Racemic)

### Preparation of 3-amino-2-[4-(aminomethyl)phenyl]-N-[3-(1H-pyrazol-4-yl)-1H-indol-7-yl]propanamide

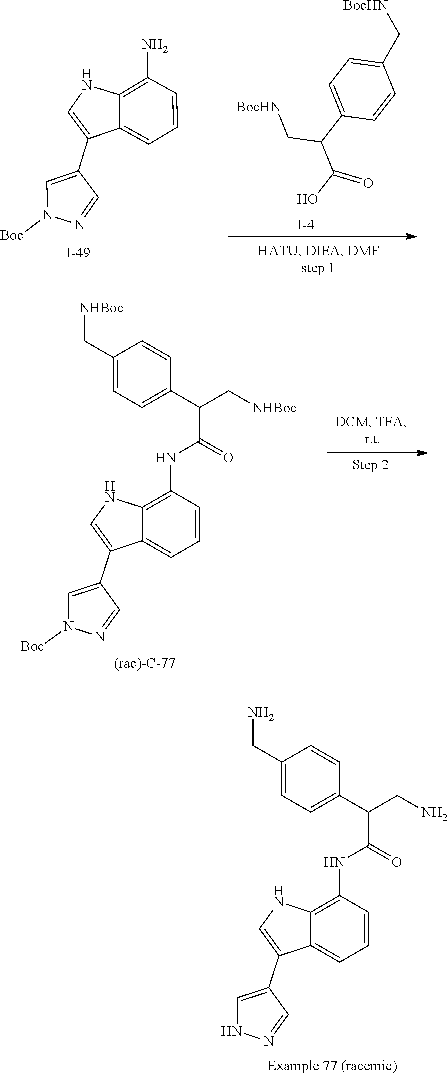

Step 1: To a stirred mixture of acid I-4 (80 mg, 0.20 mmol, 1 equiv) and DIEA (78.64 mg, 0.61 mmol, 3 equiv) in DMF (1.0 mL) was added amine I-49 (61.72 mg, 0.21 mmol, 1.02 equiv), and HATU (154.23 mg, 0.41 mmol, 2 equiv) in portions at room temperature under nitrogen atmosphere. The resulting mixture was stirred for 2.0 h at room temperature under nitrogen atmosphere. The resulting mixture was diluted with water (10 mL). The resulting mixture was extracted with CH₂Cl₂ (3×10 mL). The combined organic layers were washed with water (3×10 mL), dried over anhydrous Na₂SO₄. After filtration, the filtrate was concentrated under reduced pressure. The residue was purified by prep-TLC with CH₂Cl₂/methanol (20:1, v/v) to afford racemic amide C-77 (130 mg, 94.99%) as a yellow oil. LCMS: m/z (ESI), [M−+H]⁺=675.40.

Step 2: A solution of racemic amide C-77 (105 mg, 0.16 mmol, 1 equiv) and TFA (0.5 mL) in DCM (2.0 mL) was stirred for 2 h at room temperature under air atmosphere. After reaction, the resulting mixture was concentrated under reduced pressure to afford 80 mg crude. The crude was diluted with methanol (2 mL). The mixture was basified to pH 9 with NH₃ (aq). The crude product (80 mg) was purified by prep-HPLC with the following conditions (Column: XBridge Shield RP18 OBD 30*150 mm, 5 um; Mobile Phase A: water (10 mmol/L NH₄HCO₃+0.1% NH₃ aq), Mobile Phase B: ACN; Flow rate: 60 mL/min; Gradient: 5% B to 28% B in 8 min; wavelength: 220 nm; t_{R}=7.83 min) to afford Example 77, racemic (22.6 mg, 38.79%) as a white solid. LCMS: m/z (ESI), [M+H]⁺=375.15. ¹H NMR (CD₃OD, 400 MHZ) δ 3.02 (1H, dd), 3.43 (1H, dd), 3.88 (2H, s), 3.95 (1H, dd), 7.05 (2H, m), 7.41 (2H, d), 7.49 (3H, m), 7.64 (1H, dd), 7.91 (2H, s).

### Example 78 (Racemic)

### Preparation of 3-amino-2-{3-[(dimethylamino)methyl]phenyl}-N-[3-(1H-pyrazol-4-yl)-1H-indol-7-yl]propanamide

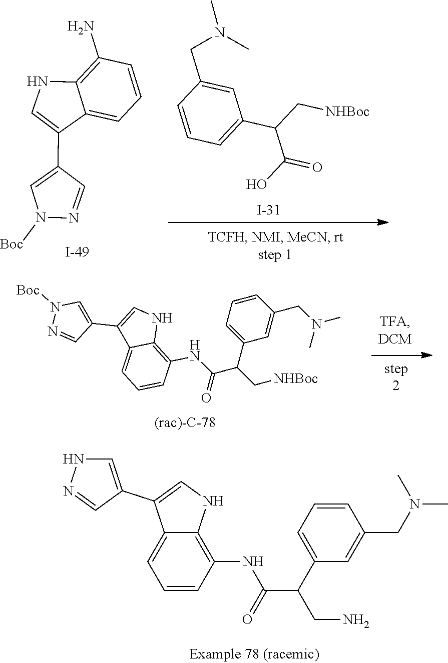

Step 1: To a solution of acid I-31 (90.55 mg, 0.29 mmol, 1.2 equiv) and amine I-49 (73 mg, 0.25 mmol, 1 equiv) in ACN (10 mL) were added TCFH (82.38 mg, 0.29 mmol, 1.2 equiv) and NMI (80.36 mg, 0.98 mmol, 4 equiv) at 25° C. under N₂ atmosphere. The mixture was stirred for 2 hours at 25° C. The resulting mixture was filtered and the filtered cake was washed with methanol (3×100 mL). The filtrate was concentrated under reduced pressure. The residue was purified by prep-TLC with DCM/methanol (10:1, v/v) to afford racemic amide C-78 (160 mg, 85.11%) as a brown solid. LCMS: m/z (ESI), [M+H]⁺=603.40.

Step 2: To a solution of racemic amide C-78 (160 mg, 0.27 mmol, 1 equiv) in DCM (15 mL) was added TFA (5 mL) at 25° C. under N₂ atmosphere. The mixture was stirred for 2 hours at 25° C. The resulting mixture was concentrated under reduced pressure. The residue was dissolved in water (50 mL). The mixture was basified to pH=8 with NaHCO₃. The resulting mixture was concentrated under vacuum. The crude product was purified by prep-HPLC with the following conditions (NH₄HCO₃) to afford Example 78, racemic (51.3 mg, 46.98%) as a white solid. LCMS: m/z (ESI), [M+H]⁺=403.20. ¹H NMR (CD₃OD, 400 MHZ) δ 2.35 (6H, s), 3.24 (1H, dd), 3.59 (2H, m), 3.66 (1H, d), 4.16 (1H, dd), 7.08 (2H, m), 7.35 (1H, dt), 7.48 (4H, m), 7.65 (1H, dd), 7.92 (2H, s).

### Example 79 (Racemic)

### Preparation of N-(3-(1H-pyrazol-4-yl)-1H-indol-7-yl)-3-amino-2-(4-hydroxyphenyl)-propanamide

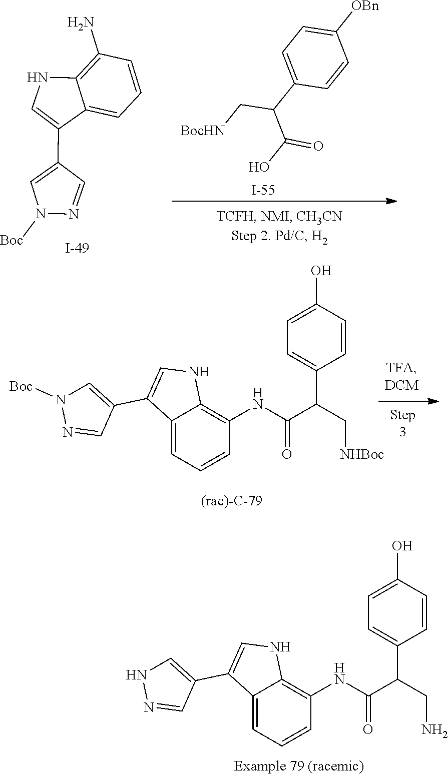

Step 1: To a stirred solution of acid I-55 (140 mg, 0.38 mmol, 1.00 equiv) and NMI (108.32 mg, 1.32 mmol, 3.5 equiv) in MeCN (3 mL) were added TCFH (158.63 mg, 0.57 mmol, 1.5 equiv) and amine I-49 (112.45 mg, 0.38 mmol, 1 equiv) in portions at room temperature under air atmosphere. The resulting mixture was stirred for 2 h at room temperature under air atmosphere. The resulting mixture was concentrated under reduced pressure. The residue was purified by silica column chromatography, eluted with PE/EA (1:1, v/v) to afford tert-butyl 4-(7-{2-[4-(benzyloxy)phenyl]-3-[(tert-butoxycarbonyl)amino]propanamido}-1H-indol-3-yl) pyrazole-1-carboxylate (226 mg, 92.00% yield) as a brown solid. LCMS: m/z (ESI), [M+H]⁺=652.40.

Step 2: A mixture of tert-butyl 4-(7-{2-[4-(benzyloxy)phenyl]-3-[(tert-butoxycarbonyl)amino]propanamido}-1H-indol-3-yl) pyrazole-1-carboxylate (216 mg, 0.33 mmol, 1 equiv) and Pd/C (10.58 mg, 0.10 mmol, 0.3 equiv) in MeOH (5 mL) was stirred for 2 h at room temperature under hydrogen atmosphere. The precipitated solids were filtered out by filtration and washed with MeOH (3×30 mL). The filtrate was concentrated under reduced pressure to afford racemic amide C-79 (72.4 mg, 38.90% yield) as a white solid. LCMS: m/z (ESI), [M+H]⁺=562.30. ¹H NMR: (DMSO-d₆, 400 MHz) δ 1.35 (9H, d), 1.62 (9H, s), 3.28 (1H, d), 3.54 (1H, s), 3.96 (1H, d), 6.73 (2H, d), 7.04 (2H, t), 7.22 (2H, d), 7.47 (1H, d), 7.60 (1H, d), 7.84 (1H, d), 8.26 (1H, s), 8.49 (1H, s), 9.32 (1H, d), 9.91 (1H, s), 10.85 (1H, s).

Step 3: A solution of racemic amide C-79 (72 mg, 0.13 mmol, 1 equiv) and TFA (1 mL) in DCM (3 mL) was stirred for 2 h at room temperature under air atmosphere. The resulting mixture was concentrated under reduced pressure. The residue was dissolved in DMF (1 mL). The mixture was basified to pH 9 with NH₃—H₂O. and stirred for 30 min. The crude product (72 mg) was purified by prep-HPLC with the following conditions (Column: XBridge Prep OBD C18 Column, 30*150 mm, 5 um; Mobile Phase A: water (10 mmol/L NH₄HCO₃), Mobile Phase B: ACN; Flow rate: 60 mL/min; Gradient: 5% B to 25% B in 7 min, 25% B; wavelength: 220 nm; t_{R}: 7.55 min) to afford Example 79, racemic (23 mg, 49.64% yield) as a white solid. LCMS: m/z (ESI), [M+H]⁺=362.15. ¹H NMR: (DMSO-d₆, 400 MHZ) δ 2.82 (1H, m), 3.19 (1H, m), 3.76 (1H, m), 6.73 (2H, m), 6.99 (1H, t), 7.22 (2H, m), 7.45 (1H, m), 7.57 (2H, q), 7.95 (2H, s), 9.65 (1H, d), 10.79 (1H, m), 12.81 (1H, s).

### Example 80 (Racemic)

### Preparation of 3-amino-2-(4-aminophenyl)-N-[3-(1H-pyrazol-4-yl)-1H-indol-7-yl]propanamide

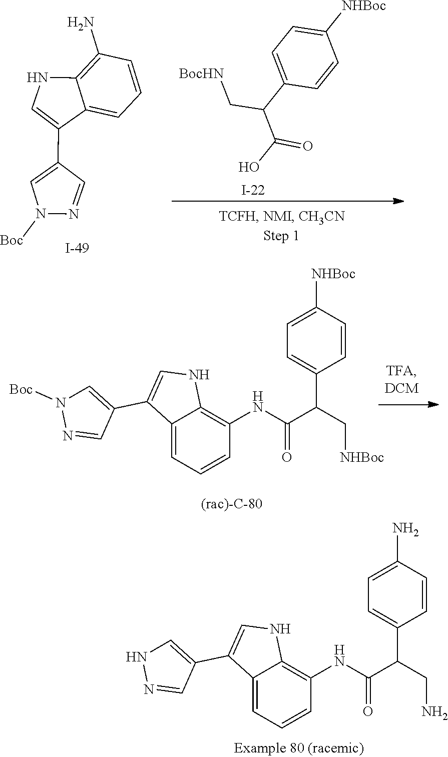

Step 1: Into a 8 mL vial were added I-22 acid (100 mg, 0.26 mmol, 1 equiv) and amine I-49 (78.42 mg, 0.26 mmol, 1 equiv) and TCFH (88.50 mg, 0.32 mmol, 1.2 equiv) and NMI (86.33 mg, 1.05 mmol, 4 equiv) in MeCN (5 mL) at room temperature. The resulting mixture was stirred for 2 h at room temperature under nitrogen atmosphere. The resulting mixture was concentrated under reduced pressure. The residue was purified by prep-TLC with CH₂Cl₂/methanol (38:1, v/v) to afford racemic amide C-80 (230 mg, 132.42%) as a yellow solid. LCMS: m/z (ESI), [M+H]⁺=661.40.

Step 2: Into a 8 mL vial were added racemic amide C-80 (220 mg, 0.33 mmol, 1 equiv) and DCM (3 mL) and TFA (1 mL) at room temperature. The resulting mixture was stirred for 1 h at room temperature under air atmosphere. The resulting mixture was concentrated under reduced pressure. The resulting mixture was diluted with DMF (2 mL) and basified to pH 9 with NH₃ (aq) at 0° C. The resulting mixture was stirred for 20 min at 0° C. under air atmosphere. The crude product (200 mg) was purified by prep-HPLC with the following conditions (Column: XBridge Prep OBD 30*150 mm, 5 um; Mobile Phase A: water (10 mmol/L NH₄HCO₃), Mobile Phase B: ACN; Flow rate: 60 mL/min; Gradient: 5% B to 25% B in 7 min; wavelength: 220 nm; t_{R}=7.03 min) to afford Example 80, racemic (31.9 mg, 25.93%) as a white solid. LCMS: m/z (ESI), [M+H]⁺=361.10. ¹H NMR (DMSO-d₆, 400 MHz) δ 2.80 (1H, dd), 3.18 (1H, m), 3.66 (1H, dd), 4.98 (2H, s), 6.53 (2H, d), 6.98 (1H, td), 7.07 (2H, d), 7.46 (1H, t), 7.56 (2H, dt), 7.95 (2H, s), 9.93 (1H, s), 10.82 (1H, d).

### Example 81 (Racemic)

### Preparation of 3-amino-2-(3-aminophenyl)-N-[3-(1H-pyrazol-4-yl)-1H-indol-7-yl]propanamide

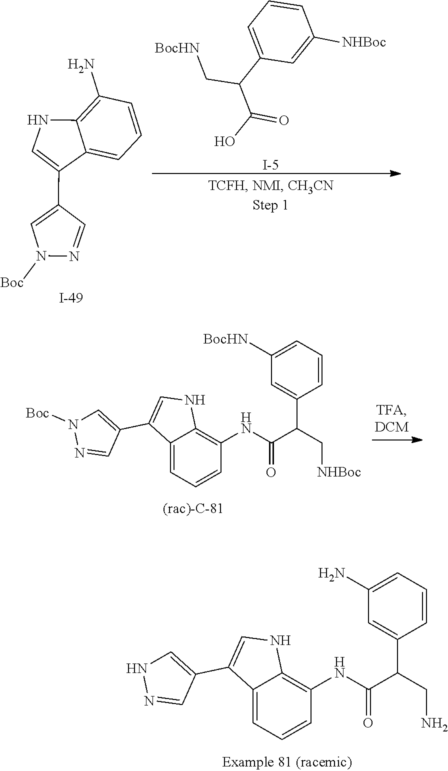

Step 1: Into a 20 mL vial were added acid I-5 (110 mg, 0.29 mmol, 1 equiv), TCFH (105.46 mg, 0.38 mmol, 1.3 equiv), NMI (94.96 mg, 1.16 mmol, 4 equiv) and amine I-49 (86.26 mg, 0.29 mmol, 1 equiv) in CH₃CN (1.5 mL) at room temperature. The resulting mixture was stirred for overnight at room temperature under nitrogen atmosphere. The resulting mixture was concentrated under reduced pressure. The residue was purified by prep-TLC with CH₂Cl₂/methanol (18:1, v/v) to afford racemic amide C-81 (70 mg, 36.64%) as a yellow solid. LCMS: m/z (ESI), [M+H]⁺=661.40. ¹H NMR (DMSO-d₆, 400 MHZ) 81.37 (9H, s), 1.48 (9H, s), 1.62 (9H, s), 3.56 (2H, s), 4.02 (1H, d), 7.05 (1H, s), 7.11 (1H, s), 7.22 (1H, d), 7.27 (1H, d), 7.48 (1H, d), 7.61 (1H, d), 7.68 (1H, s), 7.84 (1H, d), 8.26 (1H, s), 8.49 (1H, s), 9.37 (1H, s), 9.98 (1H, s), 10.82 (1H, s).

Step 2: Into a 50 mL round-bottom flask were added racemic amide C-81 (70 mg, 0.11 mmol, 1 equiv) and TFA (0.75 mL, 10.10 mmol) in DCM (1.5 mL) at room temperature. The resulting mixture was stirred for 1 h at room temperature. The mixture basified to pH 9 with NH₃ (aq). and continued to stir for 30 min. The crude product was purified by prep-HPLC with the following conditions (Column: XBridge Prep OBD C18 30*150 mm, 5 um; Mobile Phase A: water (10 mmol/L NH₄HCO₃), Mobile Phase B: ACN; Flow rate: 60 mL/min; Gradient: 5% B to 28% B in 8 min; wavelength: 220 nm; t_{R}=7.45) to afford Example 81, racemic (37.8 mg, 99.00%) as a white solid. LCMS: m/z (ESI), [M+H]⁺=361.20. ¹H NMR (CD₃OD, 400 MHZ) δ 3.03 (1H, dd), 3.40 (1H, dd), 3.85 (1H, dd), 6.69 (1H, ddd), 6.77-6.90 (2H, m), 7.01-7.19 (3H, m), 7.46 (1H, s), 7.64 (1H, dd), 7.91 (2H, s).

### Example 82 (Racemic)

### Preparation of 2-amino-2-(3,4-dimethoxyphenyl)-N-[3-(1H-pyrazol-4-yl)-1H-indol-7-yl]acetamide

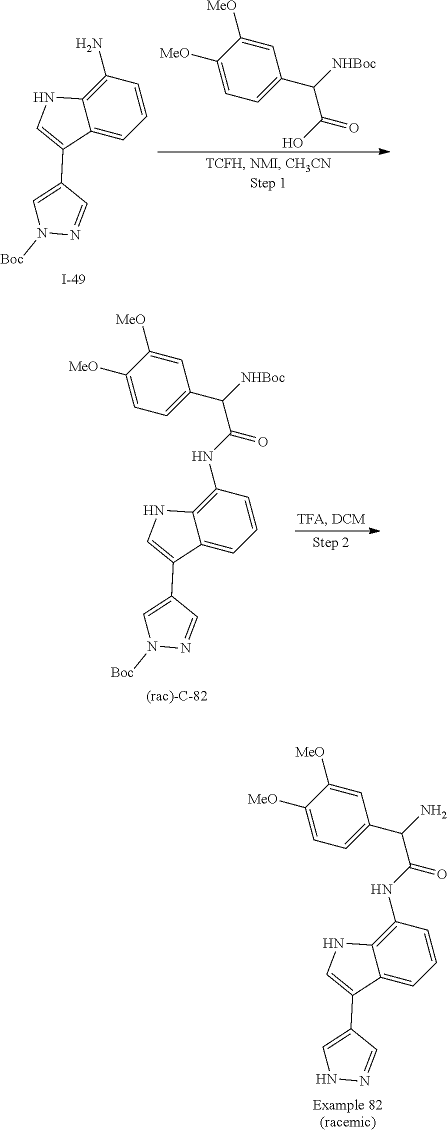

Step 1: Into a 20 mL vial were added amine I-49 (110 mg, 0.37 mmol, 1 equiv) and [(tert-butoxycarbonyl)amino](3,4-dimethoxyphenyl) acetic acid (137.75 mg, 0.44 mmol, 1.2 equiv), TCFH (124.14 mg, 0.44 mmol, 1.2 equiv), NMI (121.09 mg, 1.48 mmol, 4 equiv) in MeCN (5 mL) at room temperature. The resulting mixture was stirred for 2 h at room temperature under nitrogen atmosphere. The resulting mixture was concentrated under reduced pressure. The residue was dissolved in DCM (3 mL). The residue was purified by prep-TLC with CH₂Cl₂/methanol (30:1, v/v) to afford racemic amide C-82 (201 mg, 82.93%) as a light yellow solid. LCMS: m/z (ESI), [M+H]⁺=592.15. ¹H NMR (CD₃OD, 400 MHz) δ 1.52 (9H, s), 1.71 (9H, s), 3.86 (3H, s), 3.89 (3H, s), 5.33 (1H, s), 7.00 (1H, d), 7.13 (3H, t), 7.19 (1H, s), 7.64 (1H, s), 7.67 (1H, d), 8.16 (1H, s), 8.43 (1H, s).

Step 2: racemic amide C-82 (200 mg, 0.34 mmol, 1 equiv) in TFA (1 mL, 13.46 mmol, 39.83 equiv) and DCM (3 mL) at room temperature. The resulting mixture was stirred for 1 h at room temperature under nitrogen atmosphere. The resulting mixture was concentrated under reduced pressure. The residue was dissolved in methanol (3 mL). The mixture was basified to pH 9 with NH₃ aq. The resulting mixture was stirred for 1 h at 0° C. under nitrogen atmosphere. The crude product (120 mg) was purified by prep-HPLC with the following conditions (Column: XBridge Prep OBD C18 30*150 mm, 5 um; Mobile Phase A: water (10 mmol/L NH₄HCO₃), Mobile Phase B: ACN; Flow rate: 60 mL/min; Gradient: 5% B to 40% B in 7 min; wavelength: 220 nm; t_{R}=7.67 min) to afford Example 82, racemic (68.3 mg, 51.62%) as a white solid. LCMS: m/z (ESI), [M−NH₂]⁺=375.10. ¹H NMR (CD₃OD, 400 MHZ) δ 3.85 (3H, s), 3.89 (3H, s), 4.69 (1H, s), 6.98 (1H, s), 7.00 (1H, s), 7.20 (2H, d), 7.47 (1H, s), 7.64 (1H, d), 7.66 (1H, d), 7.92 (2H, s).

### Example 83 (Racemic)

### Preparation of 2-amino-2-(3,5-dimethoxyphenyl)-N-[3-(1H-pyrazol-4-yl)-1H-indol-7-yl]acetamide

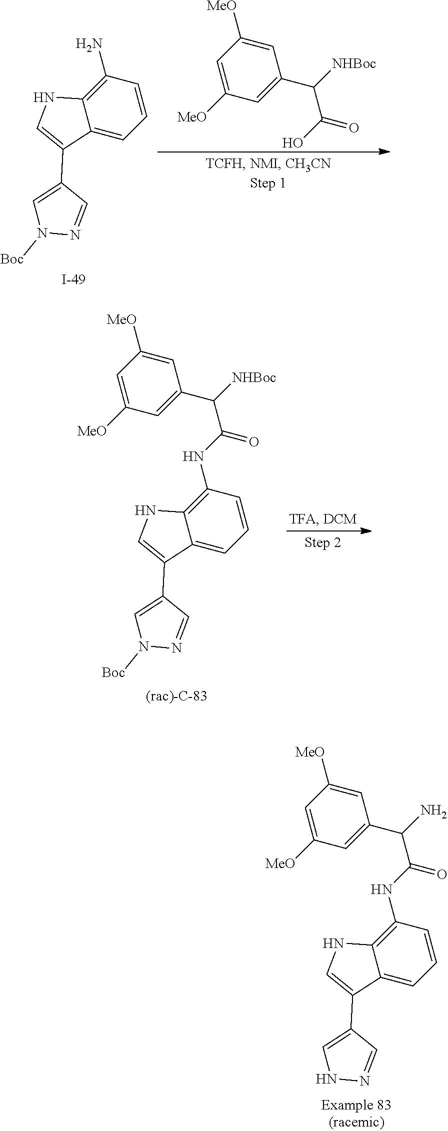

Step 1: Into a 40 mL vial were added amine I-49 (100 mg, 0.34 mmol, 1 equiv) and [(tert-butoxycarbonyl)amino](3,5-dimethoxyphenyl) acetic acid (114.79 mg, 0.37 mmol, 1.1 equiv), TCFH (112.85 mg, 0.40 mmol, 1.2 equiv), NMI (110.08 mg, 1.34 mmol, 4 equiv) in MeCN (5 mL) at room temperature. The resulting mixture was stirred for 2 h at room temperature under nitrogen atmosphere. The residue was purified by prep-TLC with CH₂Cl₂/methanol (35:1, v/v) to afford racemic amide C-83 (80 mg, 40.34%) as a light yellow solid. LCMS: m/z (ESI), [M+H]⁺=592.15.

Step 2: Into a 100 mL round-bottom flask were added racemic amide C-83 (80 mg, 0.14 mmol, 1 equiv) in DCM (3 mL) and TFA (1 mL) at room temperature. The resulting mixture was stirred for 1 h at room temperature under nitrogen atmosphere. The resulting mixture was concentrated under reduced pressure. The residue was dissolved in methanol (3 mL). The mixture was basified to pH 9 with NH₃ aq. The resulting mixture was stirred for 1 h at 0° C. under nitrogen atmosphere. The crude product (80 mg) was purified by prep-HPLC with the following conditions (Column: XBridge Prep OBD C18 30*150 mm, 5 um; Mobile Phase A: water (10 mmol/L NH₄HCO₃), Mobile Phase B: ACN; Flow rate: 60 mL/min; Gradient: 15% B to 45% B in 7 min; wavelength: 220 nm; t_{R}=7.33 min) to afford Example 83, racemic (44.8 mg, 84.65%) as a white solid. LCMS: m/z (ESI), [M+H]⁺=392.15. ¹H NMR (CD₃OD, 400 MHZ) δ 3.82 (6H, s), 4.68 (1H, s), 6.47 (1H, t), 6.76 (2H, d), 6.95-7.21 (2H, m), 7.47 (1H, s), 7.66 (1H, dd), 7.92 (2H, s).

### Example 84 (Racemic)

### Preparation of 3-amino-2-(2-methoxyphenyl)-N-[3-(1H-pyrazol-4-yl)-1H-indol-7-yl]propanamide

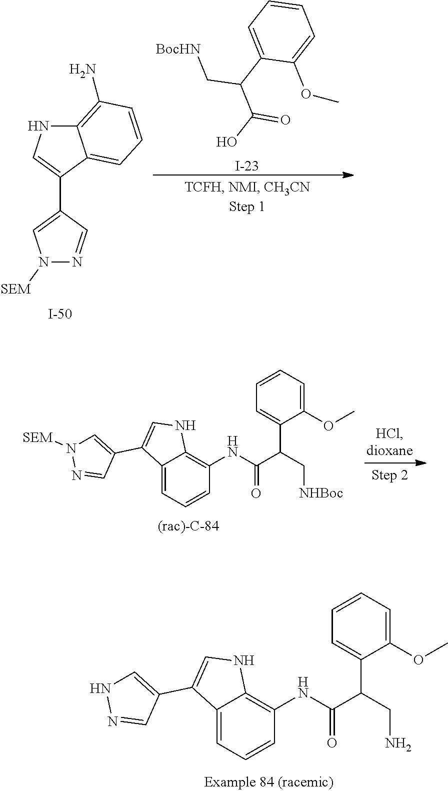

Step 1: To a stirred mixture of acid I-23 (210 mg, 0.71 mmol, 1 equiv) and NMI (175.15 mg, 2.13 mmol, 3 equiv) in DMF were added amine I-50 (256.93 mg, 0.782 mmol, 1.1 equiv) and TCFH (997.54 mg, 3.56 mmol, 5 equiv) at room temperature under nitrogen atmosphere. The resulting mixture was stirred for 2 h at room temperature under nitrogen atmosphere. The resulting mixture was diluted with water (30 mL). The aqueous layer was extracted with CH₂Cl₂ (3×20 mL). The resulting mixture was concentrated under reduced pressure. The crude product was purified by reverse phase flash with to afford racemic amide C-84 (230 mg, 53.39%) as a white solid. LCMS: m/z (ESI), [M+H]⁺=606.81.

Step 2: A mixture of racemic amide C-84 (70 mg, 0.12 mmol, 1 equiv) and HCl (gas) in 1,4-dioxane (6 mL, 0.002 mmol) was stirred for 4 h at room temperature under nitrogen atmosphere. The resulting mixture was concentrated under vacuum. The mixture/residue was basified to pH 7 with NH₃ (aq). The residue was purified by reverse flash chromatography with the following conditions: column, C18 silica; mobile phase, MeCN in water, 10% to 50% gradient in 10 min; detector, UV 254 nm to afford crude product. The crude product was purified by prep-HPLC with the following conditions (Column: XBridge Prep OBD 30*150 mm, 5 um; Mobile Phase A: water (10 mmol/L NH₄HCO₃), Mobile Phase B: ACN; Flow rate: 60 mL/min; Gradient: 15% B to 34% B in 8 min; wavelength: 254 nm; t_{R}=7.45 min) to afford Example 84, racemic (2.8 mg, 6.45%) as a white solid LCMS: m/z (ESI), [M+H]⁺=376.15. ¹H NMR (400 MHZ, DMSO-d₆) 2.88 (1H, dd), 3.13 (1H, dd), 3.87 (3H, s), 4.23 (1H, dd), 6.93 (1H, t), 6.96-7.07 (2H, m), 7.26 (1H, t), 7.37 (1H, dd), 7.47 (1H, d), 7.52-7.62 (2H, m), 7.95 (2H, s), 10.81 (1H, s), 12.81 (1H, s).

### Example 85 (Racemic)

### Preparation of 3-amino-2-(4-fluorophenyl)-N-[3-(1H-pyrazol-4-yl)-1H-indol-7-yl]propanamide

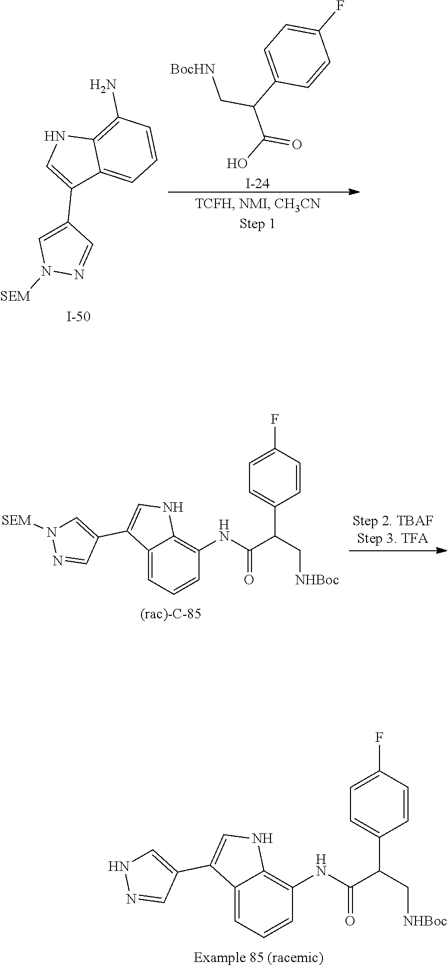

Step 1: Into a 50 mL round-bottom flask were added acid I-24 (400 mg, 1.41 mmol, 1 equiv), MeCN (10 mL), TCFH (792.32 mg, 2.82 mmol, 2 equiv), NMI (579.64 mg, 7.06 mmol, 5 equiv) and amine I-50 (417.70 mg, 2.12 mmol, 1.5 equiv) at room temperature. The resulting mixture was stirred for 8 h at room temperature. The residue was purified by prep-TLC with CH₂Cl₂/methanol (30:1, v/v) to afford racemic amide C-85 (260 mg, 39.09%) as an off-white solid. LCMS: m/z (ESI), [M+H]⁺=594.15.

Step 2: Into a 25 mL vial were added racemic amide C-85 (200 mg, 0.34 mmol, 1 equiv) and TBAF (2 mL, 0.019 mmol, 0.23 equiv) at room temperature. The resulting mixture was stirred for 8 h at 40° C. The residue was purified by prep-TLC with CH₂Cl₂/methanol (20:1, v/v) to afford tert-butyl N-[2-(4-fluorophenyl)-2-{[3-(1H-pyrazol-4-yl)-1H-indol-7-yl]carbamoyl}ethyl]carbamate (40 mg, 25.62%) as a light yellow solid. LCMS: m/z (ESI), [M+H]⁺=464.05.

Step 3. Into a 50 mL round-bottom flask were added tert-butyl N-[2-(4-fluorophenyl)-2-{[3-(1H-pyrazol-4-yl)-1H-indol-7-yl]carbamoyl}ethyl]carbamate (100 mg, 0.22 mmol, 1 equiv), TFA (1 mL) and DCM (3 mL) at room temperature. The resulting mixture was stirred for 1 h at room temperature. The resulting mixture was concentrated under reduced pressure. The resulting mixture was diluted with methanol (10 mL). The mixture was acidified to pH 7-8 with saturated NaHCO₃ (aq). The resulting mixture was extracted with CH₂Cl₂ (3×20 mL), dried over anhydrous Na₂SO₄. After filtration, the filtrate was concentrated under reduced pressure. The crude product (mg) was purified by prep-HPLC with the following conditions (Column: XBridge Shield RP18 OBD 30*150 mm, 5 um; Mobile Phase A: water 10 mmol/L NH₄HCO₃+0.1% NH₃ aq), Mobile Phase B: ACN; Flow rate: 60 mL/min; Gradient: 17% B to 40% B in 8 min; wavelength: 220 nm; t_{R}=7.83 min) to afford Example 85, racemic (2.9 mg, 3.37%) as a white solid. LCMS: m/z (ESI), [M+H]⁺=364.10. ¹H NMR (400 MHZ, CD₃OD) & 1.31 (1H, s), 3.03 (1H, dd), 3.39 (1H, s), 3.97 (1H, d), 7.07 (2H, d), 7.15 (2H, d), 7.47 (1H, s), 7.52 (2H, m), 7.64 (1H, m), 7.92 (2H, s).

### Example 86 (Racemic)

### Preparation of 3-amino-2-(4-cyanophenyl)-N-[3-(1H-pyrazol-4-yl)-1H-indol-7-yl]propanamide

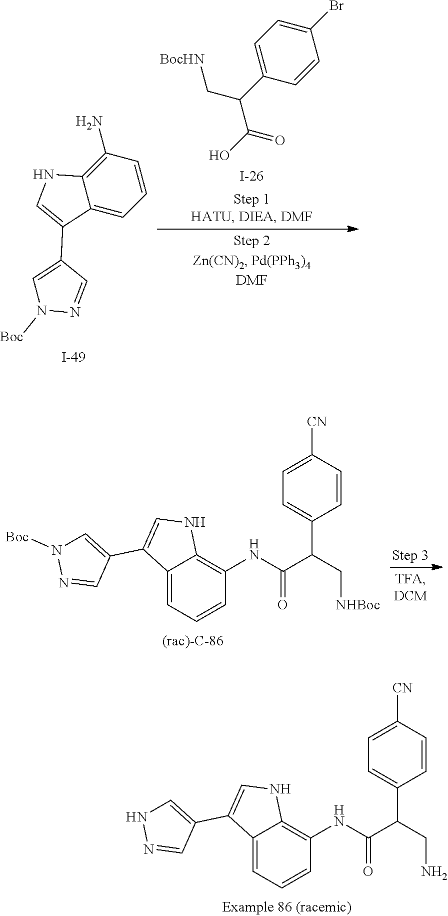

Step 1: To a stirred mixture of acid I-26 (320 mg, 0.93 mmol, 1 equiv) and amine I-49 (282.91 mg, 0.95 mmol, 1.02 equiv) in DMF (1.0 mL) was added HATU (706.99 mg, 1.86 mmol, 2 equiv) and DIEA (360.47 mg, 2.79 mmol, 3.0 equiv) dropwise at 25° C. under nitrogen atmosphere. The resulting mixture was stirred for 2 h at 25° C. under nitrogen atmosphere. The resulting mixture was diluted with water (20 mL). The resulting mixture was extracted with ethyl acetate (3×30 mL). The combined organic layers were washed with water (3×10 mL), dried over anhydrous Na₂SO₄. After filtration, the filtrate was concentrated under reduced pressure. The residue was purified by prep-TLC (CH₂Cl₂ with methanol (25:1, v/v) to afford tert-butyl 4-{7-[2-(4-bromophenyl)-3-[(tert-butoxycarbonyl)amino]propanamido]-1H-indol-3-yl}pyrazole-1-carboxylate (360 mg, 62.00%) as a yellow oil. LCMS m/z (ESI), [M-t-Bu]⁺=568.15.

Step 2: To a stirred solution of tert-butyl 4-{7-[2-(4-bromophenyl)-3-[(tert-butoxycarbonyl)amino]propanamido]-1H-indol-3-yl}pyrazole-1-carboxylate (290 mg, 0.46 mmol, 1 equiv) and Zn(CN)₂ (30.36 mg, 0.46 mmol, 1 equiv) in DMF (3.0 mL) was added Pd(PPh₃)₄ (53.66 mg, 0.046 mmol, 0.1 equiv) at 25° C. under nitrogen atmosphere. The resulting mixture was stirred for 16 h at 80° C. under nitrogen atmosphere. The resulting mixture was diluted with water (50 mL). The resulting mixture was extracted with ethyl acetate (3×40 mL). The combined organic layers were washed with water (3×10 mL), dried over anhydrous Na₂SO₄. After filtration, the filtrate was concentrated under reduced pressure. The residue was purified by prep-TLC with CH₂Cl₂/methanol (20:1, v/v) to afford racemic amide C-86 (200 mg, 75.4 8%) as a yellow solid. LCMS m/z (ESI), [M+H]⁺=471.20.

Step 3: A solution of racemic amide C-86 (110 mg, 0.19 mmol, 1 equiv) in TFA (0.5 mL) and DCM (2.0 mL) was stirred for 2 h at room temperature under nitrogen atmosphere. The resulting mixture was concentrated under reduced pressure. The resulting mixture was diluted with methanol (2.0 mL). The mixture was basified to pH 9 with NH₃ (aq). The crude product (60 mg) was purified by prep-HPLC with the following conditions (Column: XBridge Prep OBD 30*150 mm, 5 um; Mobile Phase A: water Column: XBridge Prep OBD 30*150 mm, 5 um; Mobile Phase A: water 10 (mmol/L NH₄HCO₃), Mobile Phase B: ACN; Flow rate: 60 mL/min; Gradient: 12% B to 37% B in 8 min; wavelength: 254 nm; t_{R}=7.33 min) to afford Example 86, racemic (13.2 mg, 33.00%) as a white solid. LCMS m/z (ESI), [M+H]⁺=371.10. ¹H NMR (CD₃OD, 400 MHZ) δ 3.04 (1H, dd), 3.42 (1H, m), 4.03 (1H, dd), 7.06 (2H, m), 7.47 (1H, s), 7.66 (3H, m), 7.77 (2H, d), 7.92 (2H, s).

### Example 87 (Racemic)

### Preparation of N-(3-(1H-pyrazol-4-yl)-1H-indol-7-yl)-3-amino-2-(m-tolyl)propanamide

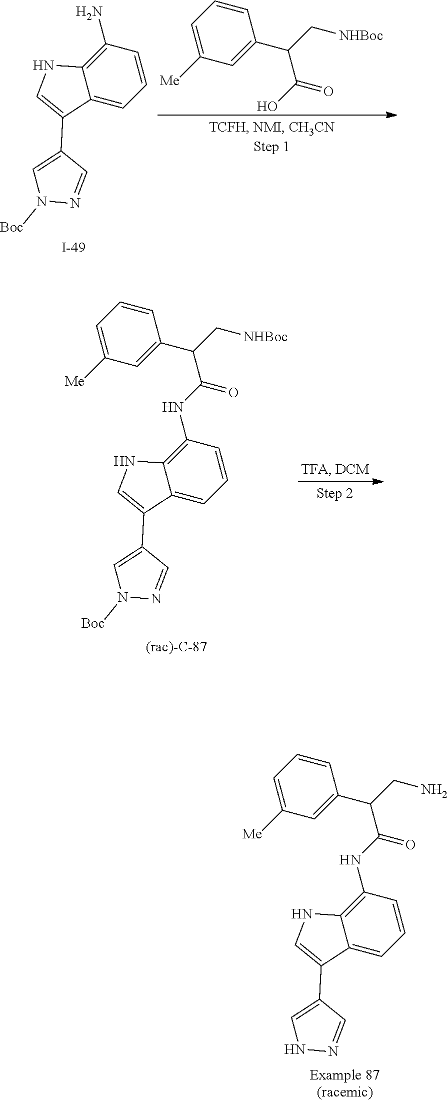

Step 1: Into a 8 mL vial were added amine I-49 (100 mg, 0.34 mmol, 1 equiv) and 4-[(tert-butoxycarbonyl)amino]-2-phenylbutanoic acid (93.63 mg, 0.34 mmol, 1.0 equiv), TCFH (141.07 mg, 0.50 mmol, 1.5 equiv) in MeCN (1 mL) was added NMI (9.63 mg, 0.12 mmol, 3.5 equiv) at room temperature. The resulting mixture was stirred for 1 h at room temperature under nitrogen atmosphere. The residue was purified by prep-TLC with CH₂Cl₂/methanol (30:1, v/v) to afford racemic amide C-87 (80 mg, 42.65%) as a yellow solid. LCMS: m/z (ESI), [M+H]⁺=560.45.

Step 2: Into a 50 mL round-bottom flask were added racemic amide C-87 (80 mg, 0.14 mmol, 1 equiv), TFA (1 mL) and DCM (3 mL) at room temperature. The resulting mixture was stirred for 1 h at room temperature. The resulting mixture was concentrated under reduced pressure. The mixture basified to pH 9 with NH₃ aq. The crude product (60 mg) was purified by prep-HPLC with the following conditions (Column: XBridge Prep OBD C18 30*150 mm, 5 um; Mobile Phase A: water (10 mmol/L NH₄HCO₃), Mobile Phase B: ACN; Flow rate: 60 mL/min; Gradient: 15% B to 45% B in 7 min; wavelength: 220 nm; t_{R}=7.10 min) to afford Example 87, racemic (40.6 mg, 78.43%) as a white solid. LCMS: m/z (ESI), [M+H]⁺=360.2. ¹H NMR (CD₃OD, 400 MHZ) δ 2.39 (3H, s), 3.09 (1H, dd), 3.47 (1H, dd), 3.98 (1H, dd), 7.03-7.10 (2H, m), 7.17 (1H, d), 7.26-7.35 (3H, m), 7.47 (1H, s), 7.64 (1H, dd), 7.91 (2H, s).

### Example 88 (Racemic)

### Preparation of 3-amino-2-[4-(2-methoxyethoxy)phenyl]-N-[3-(1H-pyrazol-4-yl)-1H-indol-7-yl]propanamide

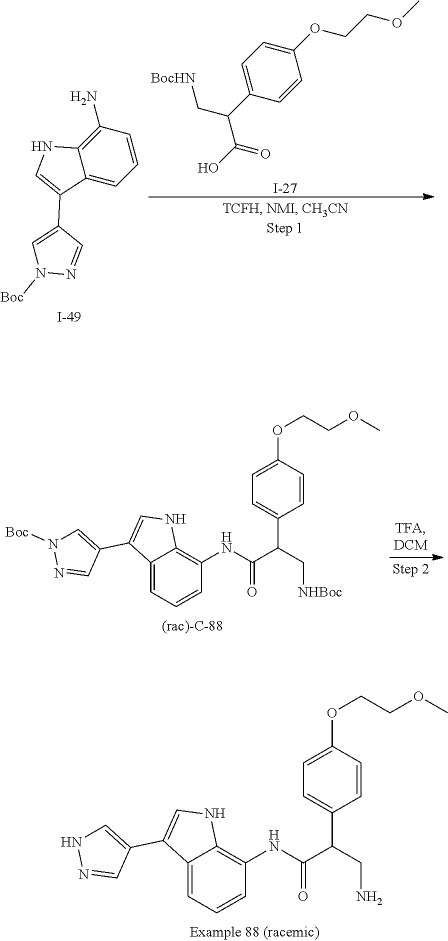

Step 1: A mixture of acid I-27 (90 mg, 0.27 mmol, 1 equiv) and amine I-49 (79.12 mg, 0.27 mmol, 1 equiv) and NMI (123.67 mg, 1.51 mmol, 5.68 equiv) and TCFH (148.81 mg, 0.53 mmol, 2 equiv) in CH₃CN (10 mL) was stirred for overnight at room temperature under nitrogen atmosphere. The resulting mixture was concentrated under reduced pressure. The reaction was quenched with water at room temperature. The resulting mixture was extracted with ethyl acetate (3×10 mL). The combined organic layers were dried over anhydrous Na₂SO₄. After filtration, the filtrate was concentrated under reduced pressure. The residue was purified by prep-TLC with CH₂Cl₂/methanol (20:1, v/v) to afford racemic amide C-88 (122 mg, 74.24%) as a yellow solid. LCMS: m/z (ESI), [M+H]⁺=620.

Step 2: A mixture of racemic amide C-88 (140 mg, 0.23 mmol, 1 equiv) and TFA (1.5 mL) in DCM (4.5 mL) was stirred for 0.5 h at room temperature under nitrogen atmosphere. The reaction mixture was basified to pH 8 with NH₃ (aq). The mixture was purified by prep-HPLC with the following conditions (Column: XBridge Prep OBD 30*150 mm, 5 um; Mobile Phase A: water (10 mmol/L NH₄HCO₃), Mobile Phase B: ACN; Flow rate: 60 mL/min; Gradient: 5% B to 40% B in 7 min; wavelength: 220 nm; t_{R}=7.550 min) to afford Example 88, racemic (42.9 mg, 42.90%) as a white solid. LCMS: m/z (ESI), [M+H]⁺=420.20. ¹H NMR (CD₃OD, 400 MHZ) δ 3.05 (1H, dd), 3.43 (4H, s), 3.70-3.80 (2H, m), 3.94 (1H, dd), 4.07-4.20 (2H, m), 6.93-7.03 (2H, m), 7.02-7.13 (2H, m), 7.34-7.45 (2H, m), 7.47 (1H, s), 7.64 (1H, dd), 7.92 (2H, s).

### Example 89 (Racemic)

### Preparation of 3-amino-2-(3-chlorophenyl)-N-[3-(1H-pyrazol-4-yl)-1H-indol-7-yl]propanamide

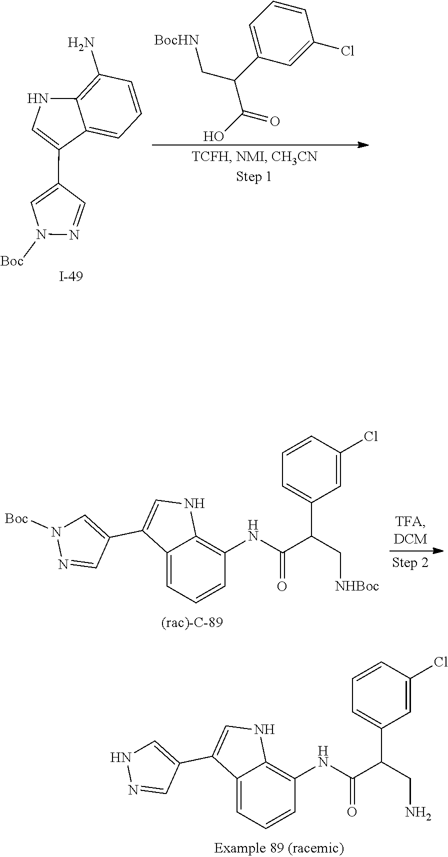

Step 1: Into a 20 mL vial were added amine I-49 (100 mg, 0.34 mmol, 1 equiv) and 3-[(tert-butoxycarbonyl)amino]-2-(3-chlorophenyl)propanoic acid (120.56 mg, 0.40 mmol, 1.2 equiv), TCFH (112.85 mg, 0.40 mmol, 1.2 equiv), NMI (110.08 mg, 1.34 mmol, 4 equiv) in MeCN (5 mL) at room temperature. The resulting mixture was stirred for 2 h at room temperature under nitrogen atmosphere. The resulting mixture was concentrated under reduced pressure. The residue was dissolved in DCM (3 mL). The residue was purified by prep-TLC with CH₂Cl₂/methanol (25:1, v/v) to afford racemic amide C-89 (170 mg, 87.43%) as a light yellow solid. LCMS: m/z (ESI), [M+H]⁺=580.15.

Step 2: Into a 25 mL round-bottom flask were added racemic amide C-89 (170 mg, 0.29 mmol, 1 equiv) in DCM (3 mL) and TFA (1 mL) at room temperature. The resulting mixture was stirred for 1 h at room temperature under nitrogen atmosphere. The resulting mixture was concentrated under reduced pressure. The mixture was basified to pH 9 with NH₃ (aq). The resulting mixture was stirred for 1 h at 0° C. under nitrogen atmosphere. The crude product (mg) was purified by prep-HPLC with the following conditions (Column: XBridge Shield RP18 OBD 19*250 mm, 10 um; Mobile Phase A: water (10 mmol/L NH₄HCO₃), Mobile Phase B: ACN; Flow rate: 60 mL/min; Gradient: 20% B to 43% B in 8 min; wavelength: 220 nm; t_{R}=7.77 min) to afford Example 89, racemic (45.5 mg, 40.87%) as a white solid. LCMS: m/z (ESI), [M+H]⁺=380.15. ¹H NMR (CD₃OD, 400 MHZ) δ 2.99-3.08 (1H, m), 3.43 (1H, dd), 3.94 (1H, ddd), 7.02-7.20 (2H, m), 7.28-7.37 (1H, m), 7.37-7.45 (2H, m), 7.47 (1H, s), 7.53 (1H, t), 7.65 (1H, dd), 7.92 (2H, s).

### Example 90 (Racemic)

### Preparation of 3-amino-2-(4-methoxyphenyl)-N-[3-(1H-pyrazol-4-yl)-1H-indol-7-yl]propanamide

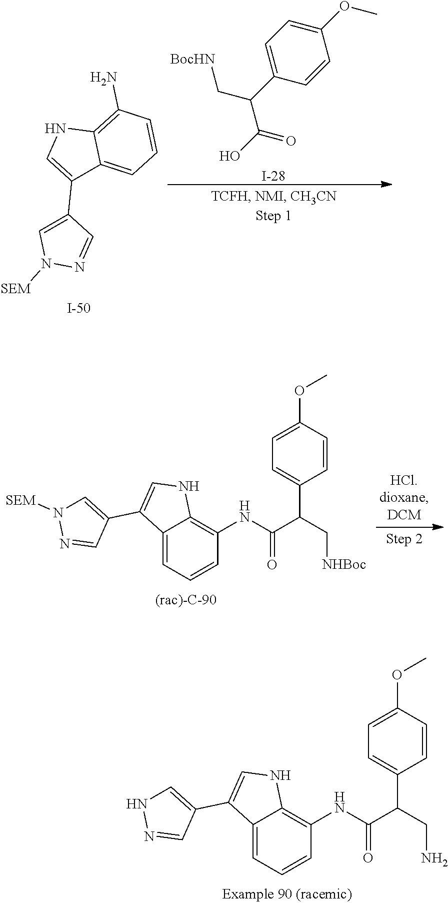

Step 1: Into a 40 mL vial were added acid I-28 (100 mg, 0.34 mmol, 1 equiv) and amine I-50 (111.23 mg, 0.34 mmol, 1 equiv) and TCFH (114.00 mg, 0.41 mmol, 1.2 equiv) and NMI (111.20 mg, 1.36 mmol, 4 equiv) in MeCN (8 mL) at room temperature. The resulting mixture was stirred for 1.5 h at room temperature under nitrogen atmosphere. The resulting mixture was concentrated under reduced pressure. The residue was purified by prep-TLC with CH₂Cl₂/methanol (35:1, v/v) to afford racemic amide C-90 (200 mg, 97.50%). LCMS: m/z (ESI), [M+H]⁺=606.45.

Step 2: Into a 100 mL round-bottom flask were added racemic amide C-90 (200 mg, 0.33 mmol, 1 equiv) and HCl (gas) in 1,4-dioxane (18 mL) at room temperature. The resulting mixture was stirred for 15 h at 40° C. under air atmosphere. The resulting mixture was concentrated under reduced pressure. The residue was dissolved in methanol (5 mL). The mixture was basified to pH 8 with saturated NaHCO₃ (aq). The aqueous layer was extracted with CH₂Cl₂/methanol (10:1, v/v) (3×50 mL). The crude product (280 mg) was purified by prep-HPLC with the following conditions to afford Example 90, racemic (32.9 mg, 25.33%) as a white solid. LCMS: m/z (ESI), [M+H]⁺=376.15. ¹H NMR (DMSO-d₆, 400 MHZ) δ 2.86 (1H, dd), 3.23 (1H, dd), 3.73 (3H, s), 3.77 (1H, dt), 6.91 (2H, m), 6.99 (1H, t), 7.35 (2H, d), 7.42 (1H, d), 7.57 (2H, m), 7.95 (2H, s), 10.04 (1H, s), 10.78 (1H, s), 12.78 (1H, s).

### Example 91 (mixture)

### Preparation of 3-amino-2-(oxan-3-yl)-N-[3-(1H-pyrazol-4-yl)-1H-indol-7-yl]propanamide

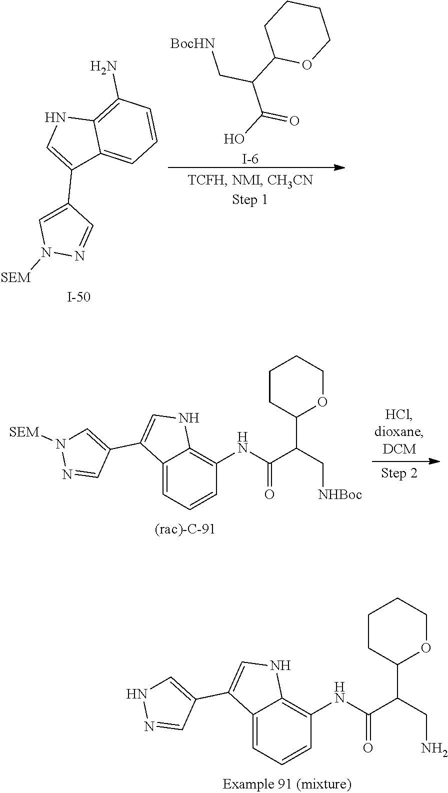

Step 1: Into a 40 mL vial were added acid I-6 (200 mg, 0.73 mmol, 1 equiv) and amine I-50 (240.36 mg, 0.73 mmol, 1 equiv) and TCFH (246.37 mg, 0.88 mmol, 1.2 equiv) and NMI (240.31 mg, 2.93 mmol, 4 equiv) in MeCN (5 mL) at room temperature. The resulting mixture was stirred for 2 h at room temperature under nitrogen atmosphere. The resulting mixture was concentrated under reduced pressure. The residue was purified by prep-TLC with CH₂Cl₂/methanol (25:1, v/v) to afford racemic amide C-91 (410 mg, 95.98%) as a white solid. LCMS: m/z (ESI), [M+H]⁺=584.45.

Step 2: A solution of racemic amide C-91 (120 mg, 0.21 mmol, 1 equiv) in DCM was stirred for 10 min at 0° C. under air atmosphere followed by the addition of HCl (gas) in 1,4-dioxane (2 mL) dropwise at 0° C. The resulting mixture was stirred for 2.3 h at room temperature under air atmosphere. The resulting mixture was concentrated under reduced pressure. The residue was dissolved in methanol (2 mL). The mixture was basified to pH 8 with NH₃ (aq). The resulting mixture was concentrated under reduced pressure. The crude product (120 mg) was purified by prep-HPLC with the following conditions (Column: Ultimate Diol, 2*25 cm, 5 um; Mobile Phase A: CO₂, Mobile Phase B: methanol (0.5% 2M NH₃-methanol); Flow rate: 50 mL/min; Gradient: isocratic 35% B; Column Temperature (35° C.); Back Pressure (100 bar); wavelength: 254 nm; t_{R}=4.5 min; Sample Solvent: methanol; Injection Volume: 1.3 mL) to afford Example 91, racemic (31.2 mg, 41.10%) as a white solid. LCMS: m/z (ESI), [M+H]⁺=354.35. ¹H NMR (CD₃OD, 400 MHz) δ 1.49 (1H, m), 1.70 (2H, m), 1.98 (2H, m), 2.56 (1H, dd), 3.05 (2H, t), 3.44 (3H, m), 3.90 (2H, m), 4.03 (1H, dt), 7.10 (1H, t), 7.18 (1H, m), 7.47 (1H, d), 7.67 (1H, d), 7.93 (2H, s).

### Example 92 (Racemic)

### Preparation of tert-butyl 4-{7-[3-amino-2-(4-acetamidophenyl)propanamido]-1H-indol-3-yl}pyrazol e-1-carboxylate

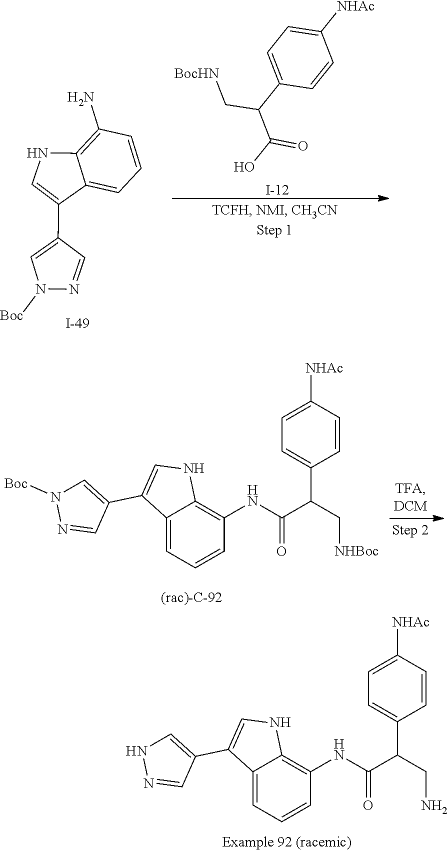

Step 1: To a stirred mixture of acid I-12 (130 mg, 0.40 mmol, 1 equiv) and NMI (99.33 mg, 1.21 mmol, 3 equiv) in CH₃CN (3.0 mL) was added TCFH (226.30 mg, 0.81 mmol, 2 equiv) and amine I-49 (122.72 mg, 0.41 mmol, 1.02 equiv) in portions at room temperature under nitrogen atmosphere. The resulting mixture was stirred for 2.0 h at room temperature under nitrogen atmosphere. The resulting mixture was diluted with water (10 mL). The resulting mixture was extracted with ethyl acetate (3×10 mL). The combined organic layers were washed with water (3×10 mL), dried over anhydrous Na₂SO₄. After filtration, the filtrate was concentrated under reduced pressure. The residue was purified by prep-TLC with CH₂Cl₂/methanol (20:1, v/v) to afford racemic amide C-92 (190 mg, 93.75%) as a yellow oil. LCMS: m/z (ESI), [M+H]⁺=603.40.

Step 2: A solution of racemic amide C-92 (100 mg, 0.20 mmol, 1 equiv) and TFA (0.5 mL) in DCM (2.0 mL) was stirred for 1.0 h at room temperature under air atmosphere. After reaction, The resulting mixture was concentrated under vacuum. The resulting mixture was diluted with methanol (1.0 mL). The mixture was basified to pH 9 with NH₃ aq (0.2 mL). The resulting solution was submitted to prep-HPLC, with the following conditions (Column: Xselect CSH C18 OBD 30*150 mm 5 um; Mobile Phase A: water (10 mmol/L NH₄HCO₃), Mobile Phase B: ACN; Flow rate: 25 mL/min; Gradient: 18% B to 37% B in 8 min; wavelength: 254 nm; t_{R}=7.28 min) to afford Example 92, racemic (28 mg, 34.97%) as a white solid. LCMS: m/z (ESI), [M+H]⁺=403.15. ¹H NMR (CD₃OD, 400 MHZ) & 2.13 (3H, s), 3.01 (1H, dd), 3.40 (1H, dd), 3.91 (1H, dd), 7.06 (2H, m), 7.44 (3H, m), 7.61 (3H, m), 7.91 (2H, s).

### Example 93 (Racemic)

### Preparation of 3-amino-2-(6-methylpyridin-2-yl)-N-[3-(1H-pyrazol-4-yl)-1H-indol-7-yl]propanamide

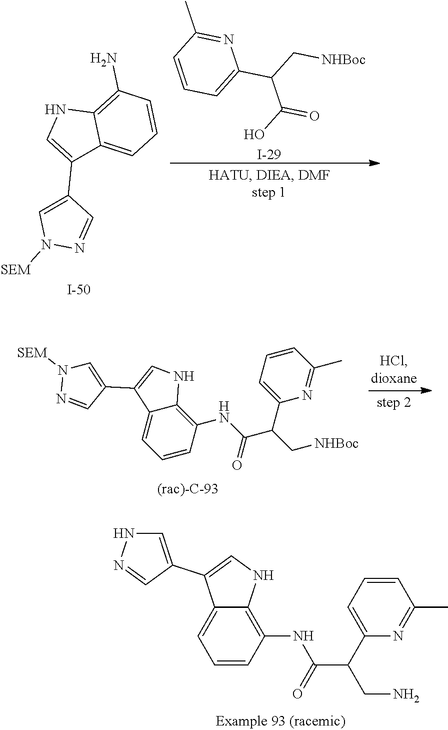

Step 1: To a stirred solution of acid I-29 (200 mg, 0.71 mmol, 1 equiv) and amine I-50 (281.24 mg, 0.856 mmol, 1.2 equiv) in DMF (5 mL) were added HATU (542.57 mg, 1.43 mmol, 2 equiv) and DIEA (276.64 mg, 2.14 mmol, 3 equiv) at room temperature under air atmosphere. The reaction mixture was stirred for overnight at room temperature under air atmosphere. The resulting mixture was concentrated under reduced pressure. The residue was purified by reverse flash chromatography with the following conditions: column, C18 silica; mobile phase, MeCN in water, 80% to 90% gradient in 10 min; detector, UV 254 nm to afford racemic amide C-93 (420 mg, 99.64%) as a yellow solid. LCMS: m/z (ESI), [M+H]⁺=591.80. ¹H NMR (DMSO-d₆, 400 MHZ) δ 0.06 (9H, s), 0.82-0.90 (2H, m), 1.36 (9H, s), 2.50 (3H, s), 3.51 (1H, dt), 3.55-3.62 (2H, m), 3.66 (1H, dd), 4.25 (1H, t), 5.44 (2H, s), 6.97-7.08 (2H, m), 7.17 (1H, d), 7.30 (1H, d), 7.45 (1H, d), 7.58 (1H, d), 7.62-7.72 (2H, m), 7.90 (1H, s), 8.27 (1H, s), 10.05 (1H, s), 10.70 (1H, s).

Step 2: A solution of racemic amide C-93 (100 mg, 0.17 mmol, 1 equiv) in HCl (gas) in 1,4-dioxane (10 mL) was stirred for overnight at room temperature under air atmosphere. The reaction mixture was concentrated under reduced pressure. The residue was purified by reverse flash chromatography with the following conditions: column, C18 silica; mobile phase, MeCN in water, 0% gradient in 10 min; detector, UV 254 nm to afford crude solid. The crude product was purified by prep-HPLC with the following conditions (Column: YMC-Actus Triart C18 ExRS, 30*150 mm, 5 um; Mobile Phase A: water (50 mmol/L NH₄HCO₃), Mobile Phase B: ACN; Flow rate: 60 mL/min; Gradient: 10% B to 30% B in 8 min; wavelength: 220 nm; t_{R}=7.89 min) to afford Example 93, racemic (5.7 mg, 9.34% yield) as a white solid. LCMS: m/z (ESI), [M+H]⁺=361.42. ¹H NMR: (DMSO-d₆, 400 MHz) δ 2.47 (3H, s), 3.04-3.15 (1H, m), 3.29 (1H, s), 4.06 (1H, dt), 7.01 (1H, t), 7.17 (1H, d), 7.31 (1H, d), 7.42 (1H, d), 7.54-7.63 (2H, m), 7.68 (1H, t), 7.96 (2H, s), 10.69-10.91 (1H, m), 12.84 (1H, s).

### Example 95 (Racemic)

### Preparation of 3-amino-2-cyclopentyl-N-[3-(1H-pyrazol-4-yl)-1H-indol-7-yl]propanamide

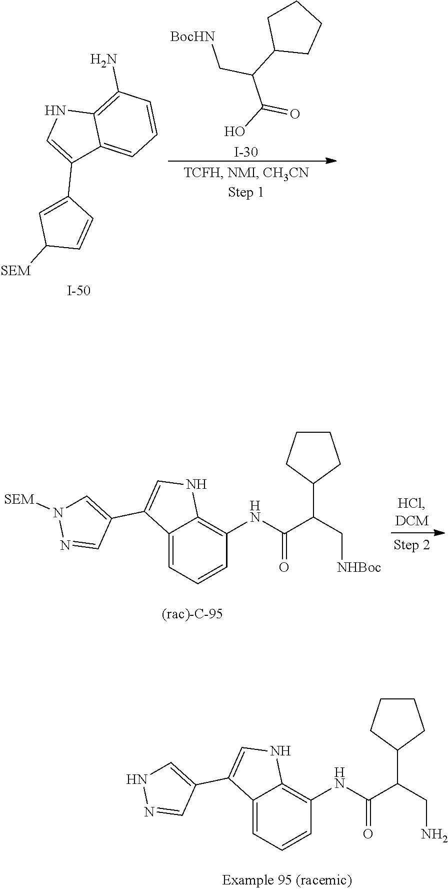

Step 1: To a stirred mixture of acid I-30 (200 mg, 0.78 mmol, 1 equiv) and amine I-50 (306.37 mg, 0.93 mmol, 1.2 equiv) in CH₃CN (10 mL) were added NMI (63.81 mg, 0.78 mmol, 1 equiv) and TCFH (261.68 mg, 0.93 mmol, 1.2 equiv) dropwise at room temperature under air atmosphere. The resulting mixture was stirred for overnight at room temperature under air atmosphere. The reaction was quenched by the addition of water (10 mL) at room temperature. The resulting mixture was extracted with CH₂Cl₂ (3×20 mL). The combined organic layers were dried over anhydrous Na₂SO₄. After filtration, the filtrate was concentrated under reduced pressure. The residue was purified by prep-TLC with DCM/methanol (20:1, v/v) to afford racemic amide C-95 (385 mg, 87.24%) as a yellow solid. LCMS: m/z (ESI), [M+H]⁺=568.80.

Step 2: A solution of racemic amide C-95 (85 mg, 0.15 mmol, 1 equiv) in HCl (gas) in 1,4-dioxane (5 mL) was stirred for 4 h at room temperature under air atmosphere. The resulting mixture was concentrated under reduced pressure. The crude product was purified by prep-HPLC with the following conditions (Column: Xselect CSH C18 OBD 30*150 mm 5 um; Mobile Phase A: water (0.1% FA), Mobile Phase B: ACN; Flow rate: 60 mL/min; Gradient: 10% B to 21% B in 8 min, 21% B; Wavelength: 254; 220 nm; t_{R}=7.18 min) to afford Example 95, racemic (12.4 mg, 24.55%) as a white solid. LCMS: m/z (ESI), [M+H]⁺=338.42. ¹H NMR (DMSO-d₆, 400 MHz) δ 1.22 (1H, s), 1.47 (3H, dd), 1.63 (3H, d), 1.81-1.86 (1H, m), 1.98-2.07 (1H, m), 2.69 (1H, d), 2.98 (2H, dd), 7.01 (1H, t), 7.51 (1H, d), 7.54-7.63 (2H, m), 7.96 (2H, s), 8.42 (1H, s), 10.27 (1H, s), 11.25 (1H, s).

### Example 94 (mixture 1) and Example 96 (mixture 2)

### Preparation of N-(3-(1H-pyrazol-4-yl)-1H-indol-7-yl)-3-amino-2-(tetrahydrofuran-3-yl)propanamide

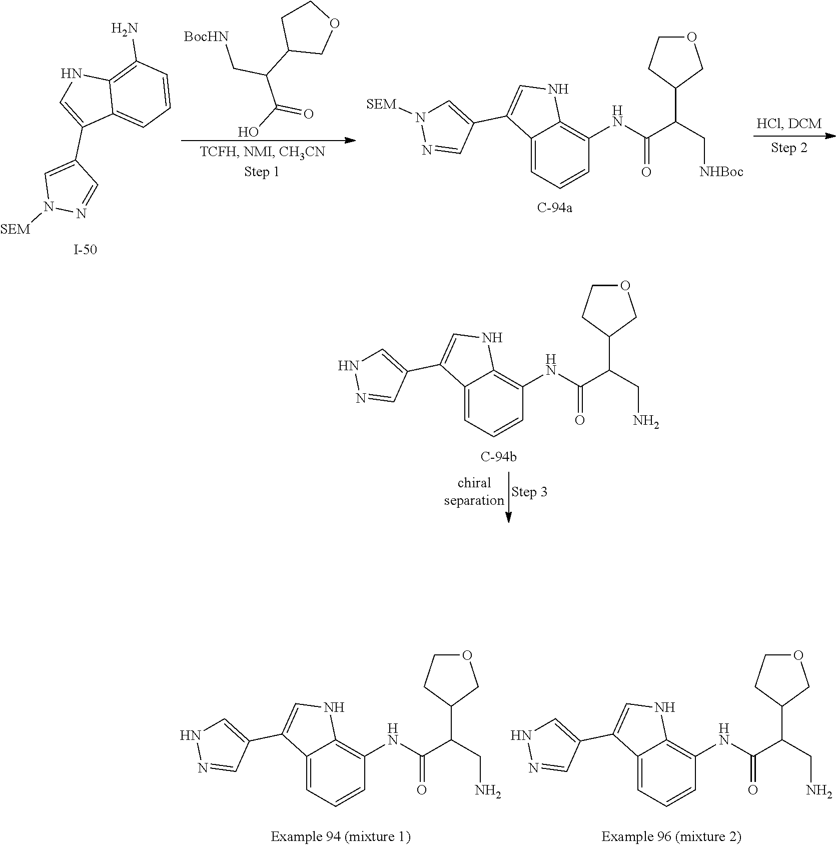

Step 1: A mixture of amine I-50 (250 mg, 0.76 mmol, 1 equiv) and 3-[(tert-butoxycarbonyl)amino]-2-(oxolan-3-yl)propanoic acid (256.55 mg, 0.99 mmol, 1.3 equiv), TCFH (427.07 mg, 1.52 mmol, 2 equiv), NMI (312.44 mg, 3.81 mmol, 5 equiv) in MeCN (4 mL) was stirred for overnight at room temperature under nitrogen atmosphere. The resulting mixture was diluted with water (20 mL), extracted with CH₂Cl₂ (3×100 mL), the combined organic layer dried over anhydrous Na₂SO₄. After filtration, the filtrate was concentrated under reduced pressure. The residue was purified by prep-TLC with CH₂Cl₂/methanol (20:1, v/v) to afford racemic amide C-94a (200 mg, 46.12%) as a off-white solid. LCMS: m/z (ESI), [M+H]⁺=570.45.

Step 2: To a stirred solution of racemic amide C-94a (100 mg, 0.18 mmol, 1 equiv) in DCM (2 mL) was added HCl (gas) in 1,4-dioxane (2 mL) dropwise at 0° C. under nitrogen atmosphere. The resulting mixture was stirred for 2 h at room temperature under nitrogen atmosphere. The resulting mixture was concentrated under vacuum. The residue was basified to pH 8 with saturated NH₃ (aq) at 0° C.

Step 3: The crude product C-94b (150 mg) was purified by prep-HPLC with the following conditions (Column: XBridge Shield RP18 OBD 30*150 mm, 5 um; Mobile Phase A: water 10 mmol/L NH₄HCO₃+0.1% NH₃ aq), Mobile Phase B: ACN; Flow rate: 60 mL/min; Gradient: 8% B to 20% B in 10 min; wavelength: 220 nm; t_{R}−1=8.88, t_{R}−2=9.53) to afford Example 94, mixture 1 (6 mg, 9.70%) as a off-white solid. LCMS: m/z (ESI), [M+H]⁺=340.10. ¹H NMR (CD₃OD, 400 MHz) δ 1.73-1.91 (1H, m), 2.22 (1H, td), 2.54-2.67 (2H, m), 2.98-3.21 (2H, m), 3.70-3.84 (2H, m), 3.86-4.02 (2H, m), 7.05-7.19 (2H, m), 7.47 (1H, s), 7.68 (1H, dd), 7.92 (2H, s) and Example 96, mixture 2 (7.6 mg, 12.06%) as a off-white solid. LCMS: m/z (ESI), [M+H]⁺=340.10. ¹H NMR (CD₃OD, 400 MHZ) δ 1.91-2.02 (1H, m), 2.07-2.20 (1H, m), 2.46-2.69 (2H, m), 2.89 (1H, dd), 3.07 (1H, dd), 3.63 (1H, dd), 3.78 (1H, q), 3.89-4.05 (2H, m), 7.05-7.21 (2H, m), 7.48 (1H, s), 7.68 (1H, dd), 7.93 (2H, s).

### Example 97 (Racemic)

### Preparation of 3-amino-2-[4-(dimethylphosphoryl)phenyl]-N-[3-(1H-pyrazol-4-yl)-1H-Indol-7-yl]propanamide

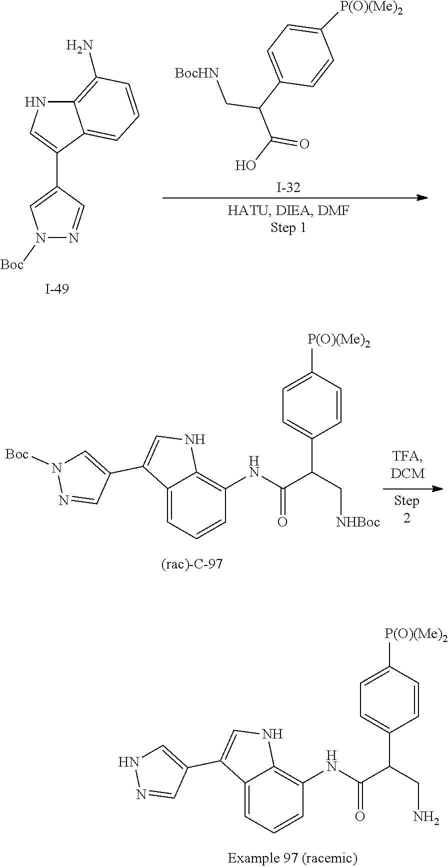

Step 1: To a stirred mixture of acid I-32 (80 mg, 0.029 mmol, 1 equiv) and amine I-49 (96.14 mg, 0.32 mmol, 1.1 equiv) in DMF (4 mL) were added HATU (222.79 mg, 0.586 mmol, 2 equiv) and DIEA (113.59 mg, 0.88 mmol, 3 equiv) in portions at 25° C. under nitrogen atmosphere. The resulting mixture was stirred for 2 h at 25° C. under nitrogen atmosphere. The resulting mixture was diluted with water (10 mL) and extracted with ethyl acetate (3×20 mL). The combined organic layers were washed with water (3×10 mL), dried over anhydrous Na₂SO₄. After filtration, the filtrate was concentrated under reduced pressure. The residue was purified by prep-TLC with CH₂Cl₂/methanol (25:1, v/v) to afford racemic amide C-97 (80 mg, 54.91%) as a yellow solid. LCMS: m/z (ESI), [M−156]⁺=466.20.

Step 2: To a stirred mixture of racemic amide C-97 (70 mg, 0.097 mmol, 1 equiv) in DCM (2.0 mL) was added TFA (0.5 mL) at 25° C. under nitrogen atmosphere. The resulting mixture was stirred for 2 h at room temperature under nitrogen atmosphere. The resulting mixture was concentrated under vacuum. The resulting mixture was diluted with methanol (2.0 mL). The mixture was neutralized to pH 7 with NH₃ aq (0.5 mL). The mixture was purified by prep-HPLC with the following conditions (Column: XBridge Prep OBD 30*150 mm, 5 um; Mobile Phase A: water (10 mmol/L NH₄HCO₃), Mobile Phase B: ACN; Flow rate: 60 mL/min; Gradient: 41% B to 61% B in 8 min; wavelength: 254 nm; t_{R}=7.65 min). After lyophilization to afford Example 97, racemic (13.2 mg, 32.45%) as a white solid. LCMS: m/z (ESI), [M+H]⁺=422.15. ¹H NMR (DMSO-d₆, 400 MHz) δ 1.63 (6H, d), 2.93 (1H, dd), 3.30 (2H, dd), 3.5-3.97 (2H, m), 7.00 (1H, t), 7.41 (1H, d), 7.57 (4H, m), 7.75 (2H, dd), 7.96 (2H, s), 10.80 (1H, m).

### Example 98 (Racemic)

### Preparation of 3-amino-N-[3-(1H-pyrazol-4-yl)-1H-indol-7-yl]-2-(pyridin-2-yl)propanamide

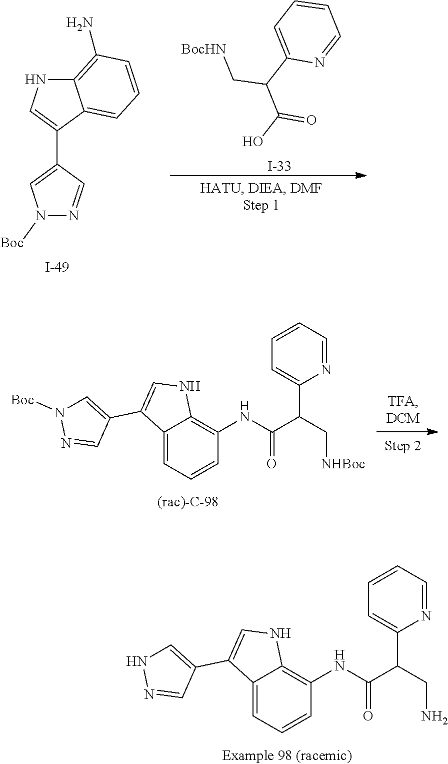

Step 1: To a stirred mixture of acid I-33 (80 mg, 0.30 mmol, 1 equiv) in DMF (1.2 mL) was added HATU (228.46 mg, 0.60 mmol, 2 equiv), DIEA (155.31 mg, 1.20 mmol, 4 equiv) and amine I-49 (90.52 mg, 0.30 mmol, 1.01 equiv) was added at 25° C. The resulting mixture was stirred at 25° C. for 2 hour under air atmosphere. The resulting mixture was diluted with water (20 mL). The resulting mixture was extracted with ethyl acetate (3×30 mL). The combined organic layers were washed with water (3×20 mL), dried over anhydrous Na₂SO₄. After filtration, the filtrate was concentrated under reduced pressure and diluted with DCM (2 mL). The residue was purified by prep-TLC with CH₂Cl₂/methanol (20:1, v/v) to afford racemic amide C-98 (80 mg, 48.72%) as a yellow solid. LCMS: m/z (ESI), [M+H]⁺=577.30.

Step 2: A mixture of racemic amide C-98 (80 mg, 0.15 mmol, 1 equiv) and in DCM (2.0 mL) and TFA (0.5 mL) was stirred for 2 h at 25° C. under nitrogen atmosphere. The resulting mixture was concentrated under reduced pressure. The resulting mixture was diluted with methanol (2.0 mL). The mixture was basified to pH 7 with NH₃ aq (0.2 mL). The crude was purified by prep-HPLC with the following conditions (Column: XBridge Prep OBD 30*150 mm, 5 um; Mobile Phase A: water (10 mmol/L NH₄HCO₃), Mobile Phase B: ACN; Flow rate: 60 mL/min; Gradient: 5% B to 30% B in 8 min; wavelength: 254 nm; t_{R}=7.4 min). After lyophilization to afford Example 98, racemic (5.2 mg, 10.26%) as a white solid. LCMS: m/z (ESI), [M+H]⁺=347.10. ¹H NMR (CD₃OD, 400 MHZ) δ 3.26 (1H, dd), 3.51 (1H, dd), 4.19 (1H, dd), 7.10 (2H, m), 7.38 (1H, m), 7.49 (1H, s), 7.59 (1H, dt), 7.66 (1H, dd), 7.88 (3H, m), 8.63 (1H, m).

### Example 99 (Racemic)

### Preparation of N-(3-(1H-pyrazol-4-yl)-1H-indol-7-yl)-2-(4-(acetamidomethyl)phenyl)-3-aminopropanamide

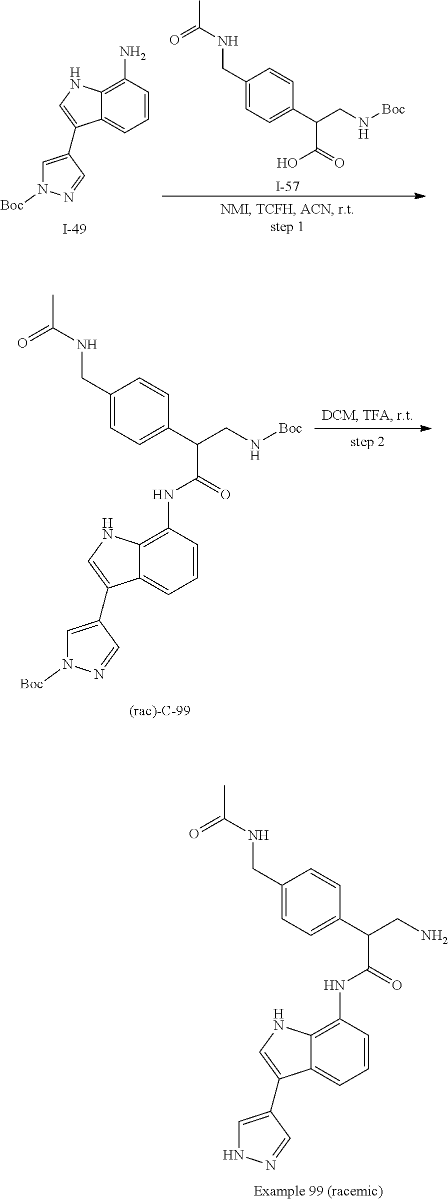

Step 1: To a stirred mixture of acid I-57 (82 mg, 0.24 mmol, 1 equiv) and NMI (60.04 mg, 0.73 mmol, 3 equiv) in MeCN (3.0 mL) was added amine I-49 (87.27 mg, 0.29 mmol, 1.2 equiv) and TCFH (136.79 mg, 0.49 mmol, 2 equiv) in portions at room temperature under air atmosphere. The resulting mixture was stirred for 2 h at room temperature under air atmosphere. After reaction, the resulting mixture was diluted with water (20 mL). The resulting mixture was extracted with EtOAc (3×10 mL). The combined organic layers were washed with water (3×10 mL), dried over anhydrous Na₂SO₄. After filtration, the filtrate was concentrated under reduced pressure. The residue was purified by prep-TLC with CH₂Cl₂/MeOH (20:1, v/v) to afford racemic amide C-99 (42 mg, 27.94% yield) as a yellow oil. LCMS: m/z (ESI), [M+Na]⁺=639.35.

Step 2: A mixture of racemic amide C-99 (42 mg, 0.068 mmol, 1 equiv) in TFA (0.5 mL) and DCM (2 mL) was stirred for 2 h at room temperature under air atmosphere. After reaction, the resulting mixture was concentrated under vacuum. The resulting mixture was diluted with MeOH (1.2 mL). The mixture was basified to pH 9 with NH₃—H₂O (0.4 mL). The crude product (40 mg) was purified by prep-HPLC with the following conditions (Column: YMC-Actus Triart C18 ExRS, 30*150 mm, 5 um; Mobile Phase A: water (10 mmol/L NH₄HCO₃), Mobile Phase B: ACN; Flow rate: 60 mL/min; Gradient: 5% B to 27% B in 8 min, 27% B; wavelength: 254 nm; t_{R}=8.07 min) to afford Example 99, racemic (8.7 mg, 30.67% yield) as a white solid. LCMS: m/z (ESI), [M+H]⁺=417.15. ¹H NMR: (CD₃OD, 400 MHz) δ 2.00 (3H, s), 3.01 (1H, dd), 3.42 (1H, dd), 3.93 (1H, dd), 4.37 (2H, s), 7.05 (2H, m), 7.33 (2H, d), 7.46 (3H, t), 7.64 (1H, dd), 7.91 (2H, s).

### Example 101 (Racemic)

### Preparation of 3-amino-2-[4-(hydroxymethyl)-3-methoxyphenyl]-N-[3-(1H-pyrazol-4-yl)-1H-indol-7-yl]propanamide

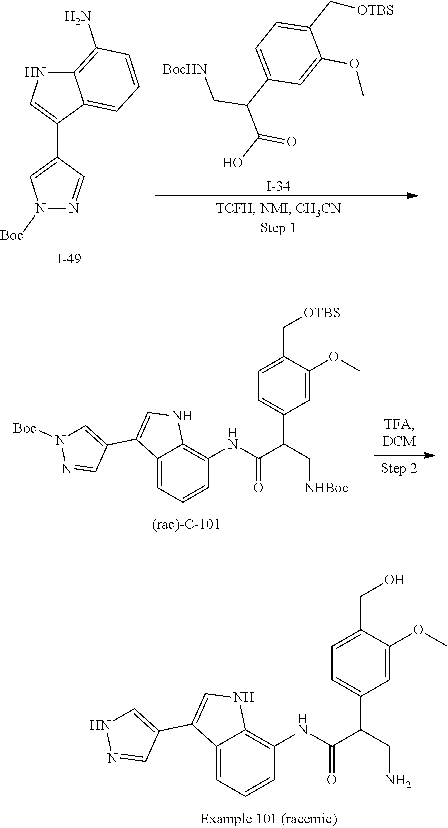

Step 1: Into a 20 mL vial were added acid I-34 (140 mg, 0.32 mmol, 1 equiv), TCFH (134.03 mg, 0.48 mmol, 1.5 equiv), NMI (104.59 mg, 1.27 mmol, 4 equiv) and amine I-49 (95.01 mg, 0.32 mmol, 1 equiv) in CH₃CN (2 mL) at room temperature. The resulting mixture was stirred for 4 h at room temperature under nitrogen atmosphere. The resulting mixture was concentrated under reduced pressure. The residue was purified by prep-TLC with CH₂Cl₂/methanol (42:1, v/v) to afford racemic amide C-101 (50 mg, 21.81%) as a yellow oil. LCMS: m/z (ESI), [M+H]⁺=720.30. ¹H NMR (DMSO-d₆, 400 MHZ) δ 0.07 (6H, d), 0.90 (9H, d), 1.36 (9H, s), 1.62 (9H, s), 3.70 (1H, s), 3.75 (1H, s), 3.81 (3H, s), 4.08 (1H, t), 4.65 (2H, s), 6.79 (1H, s), 7.31 (2H, d), 7.44 (2H, d), 7.61 (1H, d), 7.84 (1H, d), 8.26 (1H, s), 8.49 (1H, s).

Step 2: Into a 50 mL round-bottom flask were added racemic amide C-101 (50 mg, 0.069 mmol, 1 equiv) and TFA (0.75 mL) in DCM (1.5 mL) at room temperature. The resulting mixture was stirred for 1 h at room temperature. The resulting mixture was concentrated under reduced pressure. The mixture was basified to pH 9 with NH₃ (aq). The crude product was purified by prep-HPLC with the following conditions (Column: Xselect CSH C18 OBD 30*150 mm 5 um; Mobile Phase A: water (0.1% FA), Mobile Phase B: ACN; Flow rate: 60 mL/min; Gradient: 7% B to 18% B in 7 min; wavelength: 254; 220 nm; t_{R}=6.5 min) to afford a crude solid. The crude solid was repurified by prep-HPLC with the following conditions (Column: XBridge Shield RP18 OBD 30*150 mm, 5 um; Mobile Phase A: water 10 mmol/L NH₄HCO₃+0.1% NH₃ aq), Mobile Phase B: ACN; Flow rate: 60 mL/min; Gradient: 5% B to 35% B in 8 min; wavelength: 220 nm; t_{R}=8.55 min) to afford Example 101, racemic (3.5 mg, 12.43%) as a yellow solid. LCMS: m/z (ESI), [M+H]⁺=406.20. ¹H NMR (CD₃OD, 400 MHZ) & 3.07 (1H, dd), 3.46 (1H, d), 3.89 (3H, s), 3.96 (1H, dd), 4.64 (2H, s), 7.04-7.11 (4H, m), 7.39 (1H, d), 7.47 (1H, s), 7.65 (1H, dd), 7.92 (2H, s).

### Example 102 (Racemic)

### Preparation of 3-amino-2-(5-methoxypyridin-3-yl)-N-[3-(1H-pyrazol-4-yl)-1H-indol-7-yl]propanamide

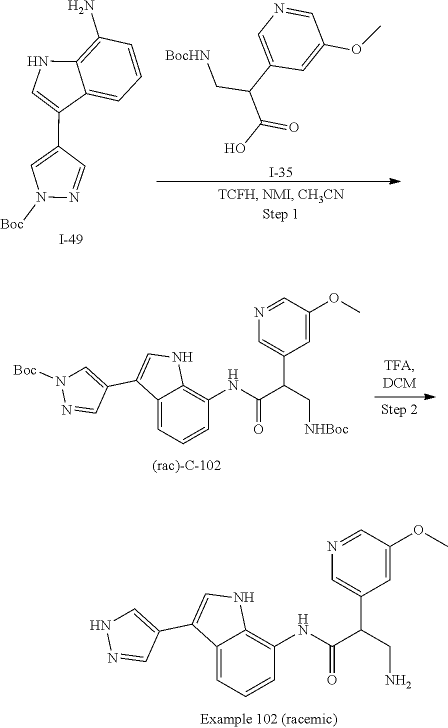

Step 1: Into a 40 mL vial were added acid I-35 (35.00 mg, 0.12 mmol, 1 equiv) and amine I-49 (35.24 mg, 0.12 mmol, 1 equiv), TCFH (39.77 mg, 0.14 mmol, 1.2 equiv), NMI (38.79 mg, 0.47 mmol, 4 equiv) in MeCN (5 mL) at room temperature. The resulting mixture was stirred for 2 h at room temperature under nitrogen atmosphere. The resulting mixture was concentrated under reduced pressure. The residue was dissolved in DCM (2 mL). The residue was purified by prep-TLC with (CH₂Cl₂/methanol (8:1, v/v) to afford racemic amide C-102 (60 mg, 88.09%) as a light yellow solid. LCMS: m/z (ESI), [M+H]⁺=477.20.

Step 2: Into a 100 mL round-bottom flask were added racemic amide C-102 (80 mg, 0.14 mmol, 1 equiv) in DCM (1 mL) and TFA (0.3 mL) at room temperature. The resulting mixture was stirred for h at room temperature under nitrogen atmosphere. The resulting mixture was concentrated under reduced pressure. The residue was dissolved in methanol (1 mL). The mixture was basified to pH 9 with NH₃ (aq). The crude product was purified by prep-HPLC with the following conditions (Column: XBridge Shield RP18 OBD 30*150 mm, 5 um; Mobile Phase A: water 10 mmol/L NH₄HCO₃), Mobile Phase B: ACN; Flow rate: 60 mL/min; Gradient: 10% B to 32% B in 8 min; wavelength: 220 nm; t_{R}=7.17 min) to afford Example 102, racemic (15 mg, 28.72%) as a white solid. LCMS: m/z (ESI), [M+H]⁺=377.20. ¹H NMR (CD₃OD, 400 MHZ) δ 3.08 (1H, dd), 3.47 (1H, dd), 3.91 (3H, dd), 4.00 (1H, dd), 6.95-7.15 (2H, m), 7.48 (1H, s), 7.55 (1H, t), 7.61-7.71 (1H, m), 7.92 (2H, s), 8.22 (1H, d), 8.26 (1H, d).

### Example 103 (Racemic)

### Preparation of 3-amino-2-(1-methyl-2-oxopyridin-4-yl)-N-[3-(1H-pyrazol-4-yl)-1H-indol-7-yl]propanamide

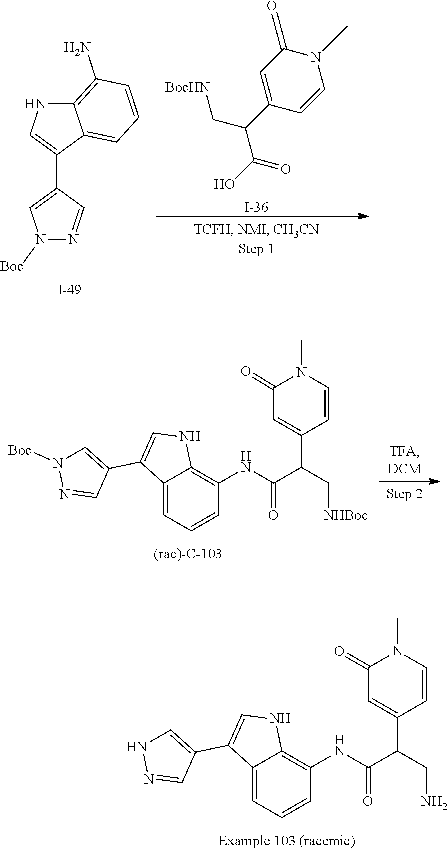

Step 1: Into a 20 mL vial were added acid I-36 (158.92 mg, 0.54 mmol, 1 equiv) and amine I-49 (160 mg, 0.54 mmol, 1.00 equiv) and HATU (305.87 mg, 0.80 mmol, 1.5 equiv) and triethylamine (162.81 mg, 1.61 mmol, 3 equiv) and DMF (5 mL) at room temperature. The resulting mixture was stirred for 1.5 h at room temperature under nitrogen atmosphere. The resulting mixture was diluted with ethyl acetate (100 mL). The resulting mixture was washed with 1×60 mL of saturated NaHCO₃ (aq) and 2×60 mL of water and 1×40 mL of saturated NaCl (aq). The organic layer was dried over anhydrous Na₂SO₄, concentrated under reduced pressure. The residue was purified by prep-TLC with CH₂Cl₂/methanol (17:1, v/v) to afford racemic amide C-103 (100 mg, 32.34%) as a yellow solid. LCMS: m/z (ESI), [M+H]⁺=577.40.

Step 2: Into a 100 mL vial were added racemic amide C-103 (80 mg, 0.14 mmol, 1 equiv) and HCl (gas) in 1,4-dioxane (1 mL) and DCM (3 mL) at room temperature. The resulting mixture was stirred for 30 min at room temperature under air atmosphere. The resulting mixture was concentrated under reduced pressure. The residue was dissolved in DMF (2 mL). The mixture was basified to pH 9 with NH₃ (aq). The resulting mixture was stirred for 30 min at 0° C. under air atmosphere. The crude product (60 mg) was purified by prep-HPLC with the following conditions (Column: XBridge Prep OBD 30*150 mm, 5 um; Mobile Phase A: water (10 mmol/L NH₄HCO₃), Mobile Phase B: ACN; Flow rate: 25 mL/min; Gradient: 2% B to 25% B in 8 min; wavelength: 254 nm; t_{R}=9.73 min) to afford Example 103, racemic (14.3 mg, 25.30%) as a white solid. LCMS: m/z (ESI), [M+H]⁺=377.20. ¹H NMR (DMSO-d₆, 400 MHZ) δ 2.88 (1H, dd), 3.19 (1H, dd), 3.38 (3H, s), 3.64 (1H, dd), 6.30 (1H, dd), 6.38 (1H, d), 7.00 (1H, td), 7.38 (1H, d), 7.60 (3H, m), 7.96 (2H, s), 10.75 (1H, s), 12.83 (1H, s).

### Example 104 (Racemic)

### Preparation of 2-amino-2-(4-methoxyphenyl)-N-[3-(1H-pyrazol-4-yl)-1H-indol-7-yl]acetamide

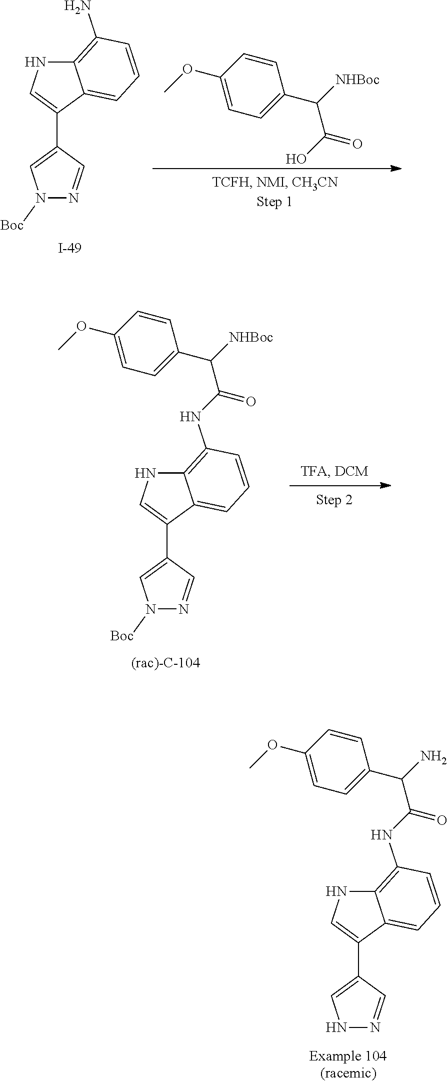

Step 1: A solution of amine I-49 (110 mg, 0.37 mmol, 1 equiv) and [(tert-butoxycarbonyl)amino](4-methoxyphenyl) acetic acid (124.46 mg, 0.44 mmol, 1.2 equiv), TCFH (155.17 mg, 0.55 mmol, 1.5 equiv), NMI (105.95 mg, 1.29 mmol, 3.5 equiv) in MeCN (3 mL) was stirred for 1 h at room temperature under nitrogen atmosphere. The residue was purified by prep-TLC with CH₂Cl₂/methanol (30:1, v/v) to afford racemic amide C-104 (140 mg, 67.61%) as a brown yellow solid. LCMS: m/z (ESI), [M+H−^{t}Bu]⁺=506.25. ¹H NMR (CD₃OD, 400 MHZ) δ 1.51 (9H, s), 1.71 (9H, s), 2.83 (1H, s), 3.82 (3H, s), 6.98 (2H, d), 7.12 (2H, d), 7.50 (2H, d), 7.65 (2H, d), 8.16 (1H, s), 8.43 (1H, s).

Step 2. A solution of racemic amide C-104 (260 mg, 0.46 mmol, 1 equiv) and TFA (1 mL) in DCM (3 mL) was stirred for 40 min at room temperature under nitrogen atmosphere. The resulting mixture was concentrated under reduced pressure. The residue was dissolved in methanol (3 mL). The mixture was basified to pH 9 with NH₃ aq at 0° C. The resulting mixture was stirred for 30 min at 0° C.° C. under nitrogen atmosphere. The crude product (230 mg) was purified by prep-HPLC with the following conditions (Column: XBridge Prep OBD C18 30*150 mm, 5 um; Mobile Phase A: water (10 mmol/L NH₄HCO₃), Mobile Phase B: ACN; Flow rate: 60 mL/min; Gradient: 10% B to 40% B in 8 min; wavelength: 220 nm; t_{R}=7.77 min) to afford Example 104, racemic (75.0 mg, 44.49%) as an off-white solid. LCMS: m/z (ESI), [M+H−NH₂]⁺=345.10. ¹H NMR (CD₃OD, 400 MHZ) δ 3.81 (3H, s), 4.70 (1H, s), 6.95-6.99 (2H, m), 7.07 (1H, t), 7.13 (1H, dd), 7.46 (1H, s), 7.49 (1H, d), 7.50 (1H, d), 7.65 (1H, dd), 7.92 (2H, s).

### Example 105 (Racemic)

### Preparation of 2-[4-(2-aminoethyl) piperazin-1-yl]-2-phenyl-N-[3-(1H-pyrazol-4-yl)-1H-indol-7-yl]acetamide

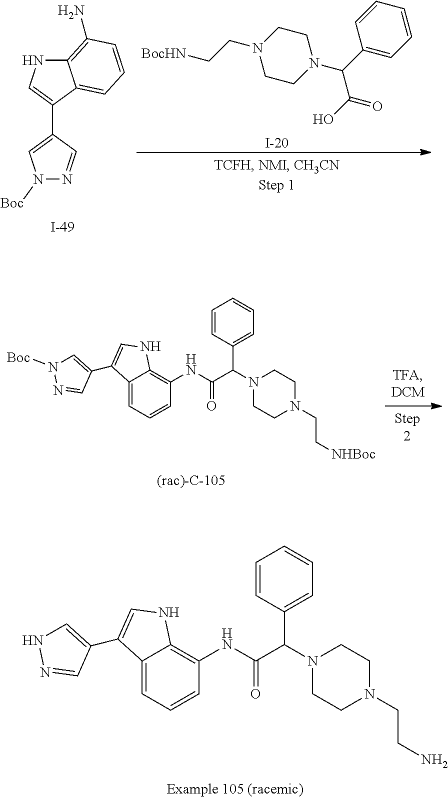

Step 1: Into a 20 mL vial were added acid I-20 (160.81 mg, 0.44 mmol, 1.2 equiv) and amine I-49 (110 mg, 0.37 mmol, 1 equiv), TCFH (124.14 mg, 0.44 mmol, 1.2 equiv), NMI (121.09 mg, 1.48 mmol, 4 equiv) in MeCN (3 mL) at room temperature. The resulting mixture was stirred for 1.5 h at room temperature under nitrogen atmosphere. The resulting mixture was concentrated under reduced pressure. The residue was dissolved in DCM (4 mL). The residue was purified by prep-TLC with CH₂Cl₂/methanol (25:1, v/v) to afford racemic amide C-105 (256 mg, 88.44%) as a light brown oil. LCMS: m/z (ESI), [M+H]⁺=644.30.

Step 2: Into a 50 mL round-bottom flask were added racemic amide C-105 (250 mg, 0.39 mmol, 1 equiv) in TFA (1 mL, 13.46 mmol) and DCM (3 mL, 47.19 mmol) at room temperature. The resulting mixture was stirred for 40 min at room temperature under nitrogen atmosphere. The resulting mixture was concentrated under reduced pressure. The residue was dissolved in methanol (5 mL). The mixture was basified to pH 9 with NH₃ aq. The crude product (200 mg) was purified by Prep-HPLC with the following conditions (Column: XBridge Shield RP18 OBD 30*150 mm, 5 um; Mobile Phase A: water (10 mmol/L NH₄HCO₃), Mobile Phase B: ACN; Flow rate: 60 mL/min; Gradient: 12% B to 37% B in 8 min; Wavelength: 220 nm; t_{R}=7.45 min) to afford Example 105, racemic (77.7 mg, 44.94%) as an off-white solid. LCMS: m/z (ESI), [M+H]⁺=444.20. ¹H NMR (CD₃OD, 400 MHZ) δ 2.33-2.69 (10H, m), 2.83 (2H, t), 4.15 (1H, s), 6.96-7.19 (2H, m), 7.24-7.51 (4H, m), 7.58-7.68 (3H, m), 7.91 (2H, s).

### Example 106 (Racemic)

### Preparation of 4-amino-2-phenyl-N-[3-(1H-pyrazol-4-yl)-1H-indol-7-yl]butanamide

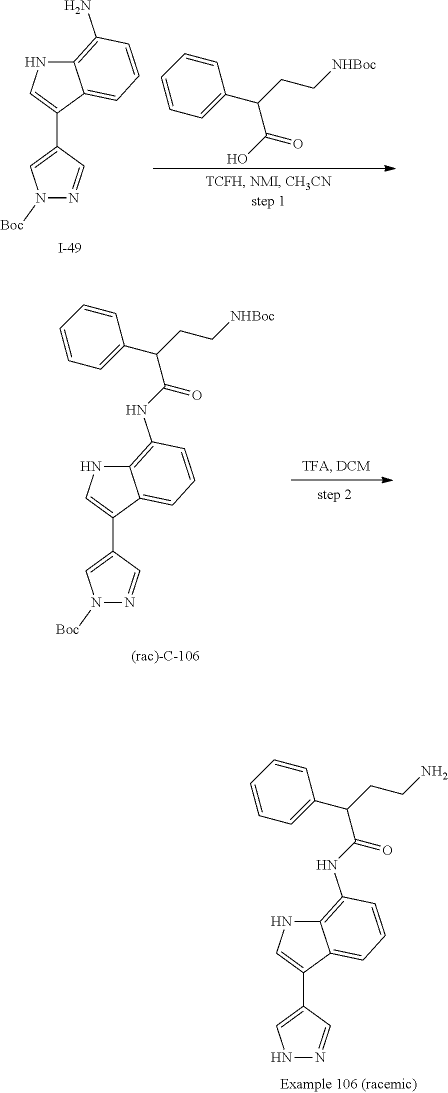

Step 1: Into a 40 mL vial were added amine I-49 (110 mg, 0.369 mmol, 1 equiv), 4-[(tert-butoxycarbonyl)amino]-2-phenylbutanoic acid (102.99 mg, 0.37 mmol, 1 equiv), TCFH (206.90 mg, 0.74 mmol, 2 equiv), NMI (151.36 mg, 1.85 mmol, 5 equiv) and MeCN (0.02 mL) at room temperature. The resulting mixture was stirred for 1 h at room temperature. The residue was purified by prep-TLC with CH₂Cl₂/methanol (45:1, v/v) to afford racemic amide C-106 (200 mg, 87.33%) as a yellow solid. LCMS: m/z (ESI), [M+H]⁺=560.3.

Step 2: Into a 40 mL vial were added racemic amide C-106 (180 mg, 0.32 mmol, 1 equiv), TFA (1 mL, 13.46 mmol) and DCM (3 mL) at room temperature. The resulting mixture was stirred for 1 h at room temperature. The mixture was basified to pH 9 with NH₃ aq. The crude product (150 mg) was purified by prep-HPLC with the following conditions (Column: XBridge Prep OBD C18 30*150 mm, 5 um; Mobile Phase A: water (10 mmol/L NH₄HCO₃), Mobile Phase B: ACN; Flow rate: 60 mL/min; Gradient: 10% B to 35% B in 8 min; wavelength: 254 nm; t_{R}=7.42 min) to afford Example 106, racemic (65.8 mg, 56.89%) as a white solid. LCMS: m/z (ESI), [M]⁺=360.20. ¹H NMR (DMSO-d₆, 400 MHZ) δ 1.81-1.91 (1H, m), 2.23 (1H, d), 2.60 (1H, d), 3.04 (1H, d), 4.00 (1H, s), 6.98 (1H, t), 7.26 (1H, d), 7.35 (2H, t), 7.46 (2H, t), 7.51-7.59 (3H, m), 7.94 (2H, s).

### Example 107 (Racemic)

### Preparation of 2-amino-2-phenyl-N-[3-(1H-pyrazol-4-yl)-1H-indol-7-yl]propanamide

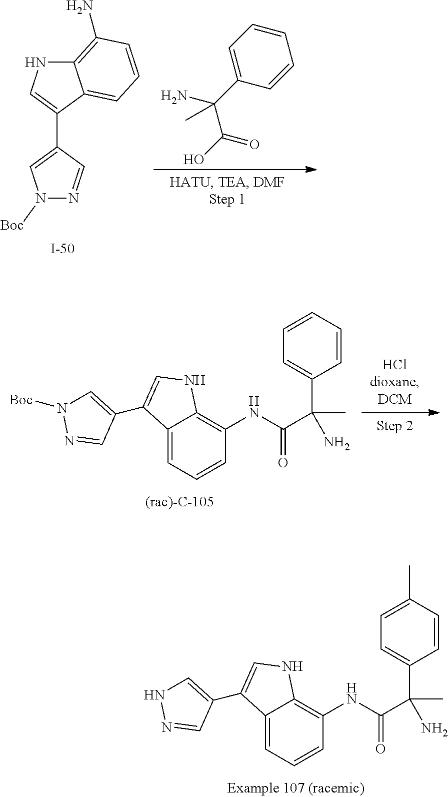

Step 1: Into a 8 mL vial were added amine I-50 (150 mg, 0.46 mmol, 1 equiv) HATU (260.44 mg, 0.69 mmol, 1.50 equiv), triethylamine (138.62 mg, 1.37 mmol, 3 equiv) and 2-amino-2-phenylpropanoic acid (105.61 mg, 0.64 mmol, 1.4 equiv) in DMF (2 mL) at room temperature. The resulting mixture was stirred for 4 h at room temperature under nitrogen atmosphere. The residue was purified by prep-TLC with CH₂Cl₂/methanol (30:1, v/v) to afford racemic amide C-107 (110 mg, 50.64%) as a yellow oil. LCMS: m/z (ESI), [M+H]⁺=476.35.

Step 2: Into a 50 mL vial were added racemic amide C-107 (100 mg, 0.21 mmol, 1 equiv) and HCl (gas) in 1,4-dioxane (2.5 mL, 0.53 mmol, 2.5 equiv) at room temperature. The resulting mixture was stirred for 1 h at room temperature. The residue was purified by reverse flash chromatography with the following conditions: column, C18 silica; mobile phase, ACN in water, 0% to 100% gradient in 40 min; detector, UV 254 nm. The crude product was purified by prep-HPLC with the following conditions (Column: Xselect CSH C18 OBD 30*150 mm 5 um; Mobile Phase A: water (0.1% FA), Mobile Phase B: ACN; Flow rate: 60 mL/min; Gradient: 6% B to 18% B in 7 min; wavelength: 254; 220 nm; t_{R}=6.66 min) to afford Example 107, racemic (4.0 mg, 5.51%) as a white solid. LCMS: m/z (ESI), [M+H]⁺=346.10. ¹H NMR (CD₃OD, 400 MHZ) δ 2.04 (3H, s), 7.00-7.15 (2H, m), 7.42 (1H, t), 7.46-7.53 (3H, m), 7.68 (3H, td), 7.92 (2H, s), 8.53 (1H, s).

### Example 108

### Preparation of (2S)-2-amino-3-(1H-imidazol-4-yl)-N-[3-(1H-pyrazol-4-yl)-1H-indol-7-yl]propanamide

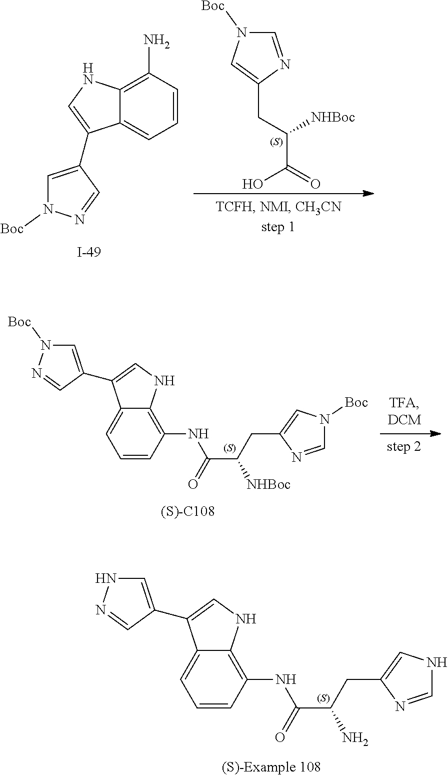

Step 1: Into a 20 mL vial were added amine I-49 (150 mg, 0.50 mmol, 1 equiv) and (2S)-2-[(tert-butoxycarbonyl)amino]-3-[1-(tert-butoxycarbonyl) imidazol-4-yl]propanoic acid (214.42 mg, 0.60 mmol, 1.2 equiv) and TCFH (169.28 mg, 0.60 mmol, 1.2 equiv) and NMI (165.12 mg, 2.01 mmol, 4 equiv) and MeCN (5 mL) at room temperature. The resulting mixture was stirred for 1 h at room temperature under nitrogen atmosphere. The resulting mixture was concentrated under reduced pressure. The residue was purified by prep-TLC with CH₂Cl₂/methanol (35:1, v/v) to afford a crude solid (250 mg). The crude product (250 mg) was purified by prep-chiral HPLC with the following conditions (Column: CHIRAL ART Cellulose-SB, 2*25 cm, 5 um; Mobile Phase A: Hex (0.2% DEA), Mobile Phase B: EtOH/DCM (1:1, v/v); Flow rate: 20 mL/min; Gradient: 25% B to 25% B in 10 min; wavelength: 220/254 nm; t_{R}−1=4.20 min; t_{R}−2=6.60 min; Sample Solvent: EtOH/DCM (1:1, v/v); Injection Volume: 0.6 mL) to afford(S)-enantiomer amide C-108 (180 mg, 56.32%) as a yellow oil. SFC: t_{R}=1.879 min, ee=97.33%.

Step 2: Into a 100 mL round-bottom were added(S)-enantiomer amide C-108 (180 mg, 0.28 mmol, 1 equiv) and TFA (1 mL) and DCM (3 mL) at room temperature. The resulting mixture was stirred for 1 h at room temperature under air atmosphere. The resulting mixture was concentrated under reduced pressure. The residue was dissolved in DMF (2 mL). The mixture was basified to pH 9 with NH₃ aq. The resulting mixture was stirred for 30 min at 0° C. under air atmosphere. The crude product (100 mg) was purified by prep-HPLC with the following conditions (Column: XBridge Shield RP18 OBD 30*150 mm, 5 um; Mobile Phase A: water (10 mmol/L NH₄HCO₃), Mobile Phase B: ACN; Flow rate: 60 mL/min; Gradient: 5% B to 25% B in 8 min; wavelength: 220 nm; t_{R}=7.53 min) to afford (2S)-2-amino-3-(1H-imidazol-4-yl)-N-[3-(1H-pyrazol-4-yl)-1H-indol-7-yl]propanamide Example 108 (40.1 mg, 41.84%) as a white solid. LCMS: m/z (ESI), [M+H]⁺=336.15. ¹H NMR (CD₃OD, 400 MHZ) δ 3.05 (1H, dd), 3.15 (1H, dd), 3.86 (1H, t), 6.99 (1H, d), 7.09 (2H, m), 7.49 (1H, s), 7.66 (1H, dd), 7.70 (1H, d), 7.92 (2H, s). SFC: t_{R}=0.414 min, ee=100%.

### Example 109

### Preparation of (2S)-2-amino-3-(1H-indol-3-yl)-N-[3-(1H-pyrazol-4-yl)-1H-indol-7-yl]propanamide

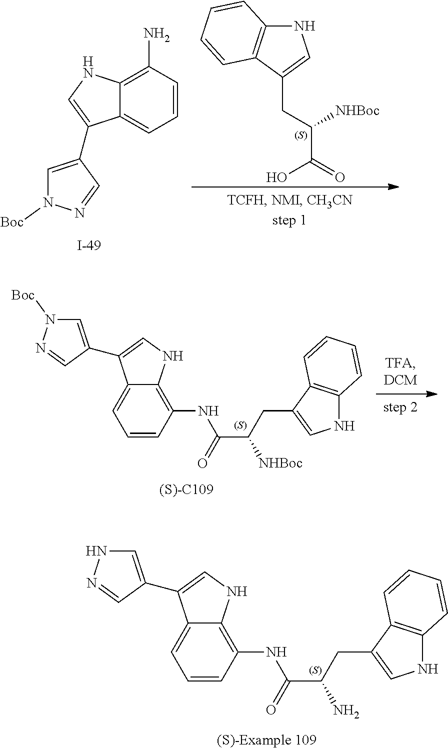

Step 1: A solution of amine I-49 (120 mg, 0.40 mmol, 1 equiv) and boc-tryptophan (183.62 mg, 0.60 mmol, 1.5 equiv), NMI (115.59 mg, 1.41 mmol, 3.5 equiv), TCFH (169.28 mg, 0.60 mmol, 1.5 equiv), in MeCN (3 mL) was stirred for 2 h at room temperature under nitrogen atmosphere. The residue was purified by prep-TLC with CH₂Cl₂/methanol (20:1, v/v) to afford tert-butyl 4-{7-[(2S)-2-[(tert-butoxycarbonyl)amino]-3-(1H-indol-3-yl)propanamido]-1H-indol-3-yl}pyrazole-1-carboxylate C-109 (160 mg, 68.04%) as a brown yellow solid. LCMS: m/z (ESI), [M+H−^{t}Bu]⁺=529.10.

Step 2: A solution of tert-butyl 4-{7-[(2S)-2-[(tert-butoxycarbonyl)amino]-3-(1H-indol-3-yl)propanamido]-1H-indol-3-yl}pyrazole-1-carboxylate C-109 (160 mg, 0.27 mmol, 1 equiv) and TFA (1.00 mL) in DCM (3 mL) was stirred for 1 h at room temperature under nitrogen atmosphere. The resulting mixture was concentrated under reduced pressure. The resulting mixture was diluted with methanol (2 mL). The mixture was basified to pH 9 with aqueous ammonia at 0° C., and continued to stir for 30 min. The crude product (120 mg) was purified by prep-HPLC with the following conditions (Column: XBridge Shield RP18 OBD 30*150 mm, 5 um; Mobile Phase A: water (10 mmol/L NH₄HCO₃+0.1% NH₃ aq), Mobile Phase B: ACN; Flow rate: 60 mL/min; Gradient: 20% B to 41% B in 8 min; wavelength: 220 nm; t_{R}=7.77 min) to afford (2S)-2-amino-3-(1H-indol-3-yl)-N-[3-(1H-pyrazol-4-yl)-1H-indol-7-yl]propanamide Example 109 (43.7 mg, 41.31%) as an off-white solid. LCMS: m/z (ESI), [M+H]⁺=385.15. ¹H NMR (CD₃OD, 400 MHZ) δ 3.22 (1H, dd), 3.37 (1H, d), 3.93 (1H, t), 6.97 (1H, dd), 7.00-7.09 (2H, m), 7.15 (1H, ddd), 7.20 (1H, s), 7.34-7.41 (2H, m), 7.63 (1H, dd), 7.69-7.74 (1H, m), 7.91 (2H, s). SFC: t_{R}=4.207, ee=100%.

### Example 110 (Enantiomer 1) and Example 111 (Enantiomer 2)

### Preparation of N-(3-(1H-pyrazol-4-yl)-1H-indol-7-yl)-2-(5-methoxypyridin-3-yl)-3-(4-methylpiperazin-1-yl)propanamide

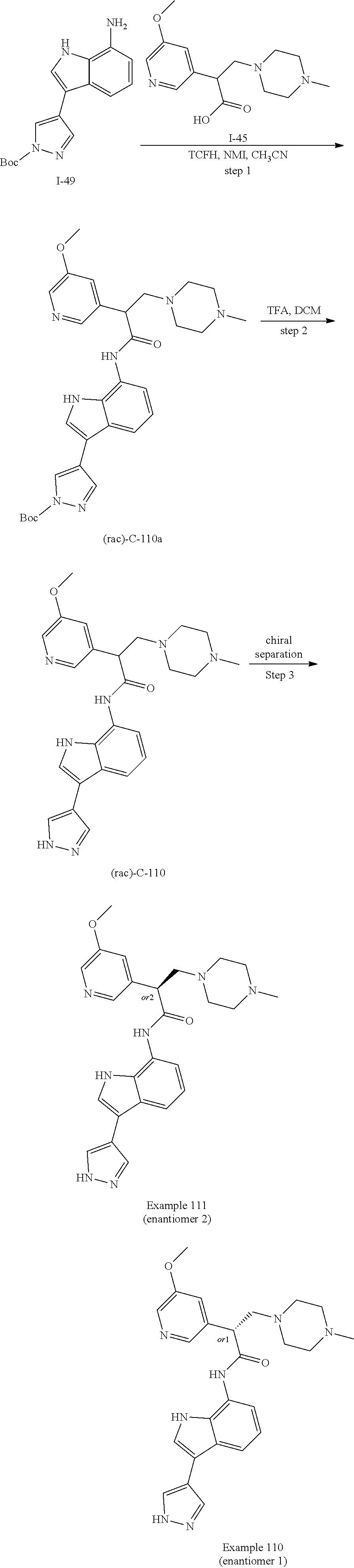

Step 1: Into a 20 mL vial were added acid I-45 (230 mg, 0.82 mmol, 1 equiv), TCFH (300.33 mg, 1.07 mmol, 1.3 equiv), NMI (270.41 mg, 3.29 mmol, 4 equiv) and amine I-49 (245.65 mg, 0.82 mmol, 1 equiv) in ACN (3 mL) at room temperature. The resulting mixture was stirred for 2 h at room temperature under nitrogen atmosphere. The resulting mixture was concentrated under vacuum. The residue was purified by prep-TLC with CH₂Cl₂/methanol (10:1, v/v) to afford racemic amide C-110a (190 mg, 41.23%) as a yellow oil. LCMS: m/z (ESI), [M+H]⁺=560.20.

Step 2: Into a 50 mL round-bottom flask were added racemic amide C-110a (190 mg, 0.16 mmol, 1 equiv) and TFA (1 mL) in DCM (1 mL) at room temperature. The resulting mixture was stirred for 1 h at room temperature. The mixture was concentrated under reduced pressure, dissolve in methanol (5 mL), basified to pH 9 with NH₃ aq. The crude product as purified by prep-HPLC with the following conditions (Column: XBridge Shield RP18 OBD 30*150 mm, 5 um; Mobile Phase A: water (10 mmol/L NH₄HCO₃), Mobile Phase B: ACN; Flow rate: 60 mL/min; Gradient: 14% B to 37% B in 8 min; wavelength: 220 nm; t_{R}=7.42 min) to afford racemic pyrazole C-110 (150 mg, 96.15%) as a white solid.

Step 3: The racemic pyrazole C-110 (150 mg) was purified by prep-HPLC with the following conditions (Column: DZ-CHIRALPAK IC-3, 4.6*50 mm, 3.0 um; Mobile Phase A: Hex (0.2% DEA): (EtOH:DCM=1:1, v/v)=65:35 (v/v); Flow rate: 1 mL/min; Gradient: 0% B to 0% B; Injection Volume: 5 ul mL) to afford Example 111, enantiomer 2 (52.8 mg, 71.45%) as a white solid. LCMS: m/z (ESI), [M+H]⁺=460.30. ¹H NMR (CD₃OD, 400 MHz) δ 2.34 (3H, s), 2.53-2.90 (9H, m), 3.36-3.42 (1H, m), 3.89 (3H, s), 4.17 (1H, dd), 7.04-7.10 (2H, m), 7.48 (1H, s), 7.58 (1H, dd), 7.65 (1H, p), 7.91 (2H, s), 8.18 (1H, d), 8.23 (1H, d), SFC: t_{R}=3.360 min, ee=100%, and Example 110, enantiomer 1 (38.1 mg, 46.40%) as a white solid. LCMS: m/z (ESI), [M+H]⁺=460.30. ¹H NMR (CD₃OD, 400 MHZ) δ 2.34 (3H, s), 2.66 (7H, dd), 2.82 (2H, s), 3.36-3.42 (1H, m), 3.89 (3H, s), 4.17 (1H, dd), 7.04-7.10 (2H, m), 7.48 (1H, s), 7.58 (1H, dd), 7.65 (1H, p), 7.91 (2H, s), 8.18 (1H, d), 8.23 (1H, d). SFC: t_{R}=4.521, ee=99.13%.

### Example 112 (Enantiomer 1) and 113 (Enantiomer 2)

### Preparation of 4-(1-((3-(1H-pyrazol-4-yl)-1H-indol-7-yl)amino)-3-amino-1-oxopropan-2-yl)benzamide

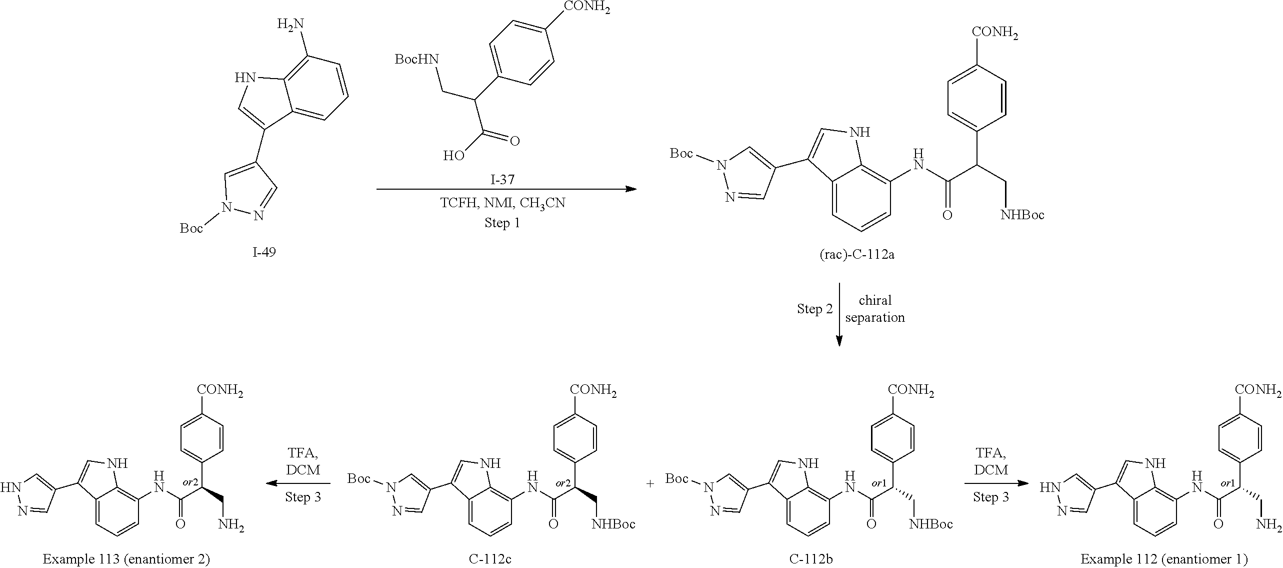

Step 1: Into a 40 mL vial were added acid I-37 (434.06 mg, 1.41 mmol, 1.2 equiv) and I-49 (350 mg, 1.17 mmol, 1.00 equiv) and TCFH (394.99 mg, 1.41 mmol, 1.2 equiv) and NMI (385.29 mg, 4.69 mmol, 4 equiv) in MeCN (8 mL) at room temperature. The resulting mixture was stirred for 1 h at room temperature under nitrogen atmosphere. The resulting mixture was concentrated under reduced pressure. The residue was purified by prep-TLC with CH₂Cl₂/methanol (15:1, v/v) to afford racemic amide C-112a (220 mg, 31.86%) as a brown solid. LCMS: m/z (ESI), [M+H]⁺=589.35.

Step 2: The racemic amide C-112a (220 mg) was purified by prep-chiral HPLC with the following conditions (Column: DZ-CHIRALPAK IC-3, 4.6*50 mm, 3.0 um; Mobile Phase A: Hex (0.2% DEA): (EtOH:DCM=1:1, v/v)=50:50 (v/v); Flow rate: 1 mL/min; Injection Volume: 5 ul mL) to afford enantiomer C-112c as a yellow solid, SFC: t_{R}=2.550 min, ee=100%, and enantiomer C-112b as a yellow solid, SFC: t_{R}=3.573 min, ce=99.47.

Step 3: Into a 50 mL round-bottom flask were added enantiomer C-112c (35 mg, 0.059 mmol, 1 equiv) and TFA (0.58 mL) in DCM (1 mL) at room temperature. The resulting mixture was stirred for 1 h at room temperature. The mixture was basified to pH 9 with NH₃ (aq). The crude product was purified by prep-HPLC with the following conditions (Column: YMC-Actus Triart C18 ExRS, 30*150 mm, 5 um; Mobile Phase A: water (10 mmol/L NH₄HCO₃), Mobile Phase B: ACN; Flow rate: 60 mL/min; Gradient: 5% B to 25% B in 7 min; wavelength: 220 nm; t_{R}=8.07 min) to afford Example 113, enantiomer 2 (19.6 mg, 99.01%) as a white solid. LCMS: m/z (ESI), [M+H]⁺=389.15. ¹H NMR (DMSO-d₆, 400 MHz) δ 2.89 (1H, dd), 3.26 (1H, dd), 3.92 (1H, dd), 6.99 (1H, td), 7.36 (1H, d), 7.50 (2H, d), 7.58 (2H, d), 7.84 (2H, d), 7.94 (2H, s).

Into a 50 mL round-bottom flask were added enantiomer C-112b (40 mg, 0.068 mmol, 1 equiv) and TFA (0.5 mL) in DCM (1 mL) at room temperature. The resulting mixture was stirred for 1 h at room temperature. The mixture was basified to pH 9 with NH₃ (aq). The crude product was purified by prep-HPLC with the following conditions (Column: XBridge Prep OBD 30*150 mm, 5 um; Mobile Phase A: water (10 mmol/L NH₄HCO₃), Mobile Phase B: ACN; Flow rate: 60 mL/min; Gradient: 2% B to 25% B in 7 min; wavelength: 220 nm; t_{R}=7.35 min) to afford Example 112, enantiomer 1 (20.8 mg, 70.05%) as a white solid. LCMS: m/z (ESI), [M+H]⁺=389.10. ¹H NMR (DMSO-d₆, 400 MHz) δ 2.90 (1H, dd), 3.27 (1H, dd), 3.92 (1H, dd), 6.99 (1H, td), 7.40 (1H, dd), 7.50 (2H, dd), 7.58 (2H, d), 7.85 (2H, d), 7.95 (2H, s).

### Example 114 (Enantiomer 1) and Example 115 (Enantiomer 2)

### Preparation of 4-(1-((3-(1H-pyrazol-4-yl)-1H-indol-7-yl)amino)-3-amino-1-oxopropan-2-yl)benzyl methylcarbamate

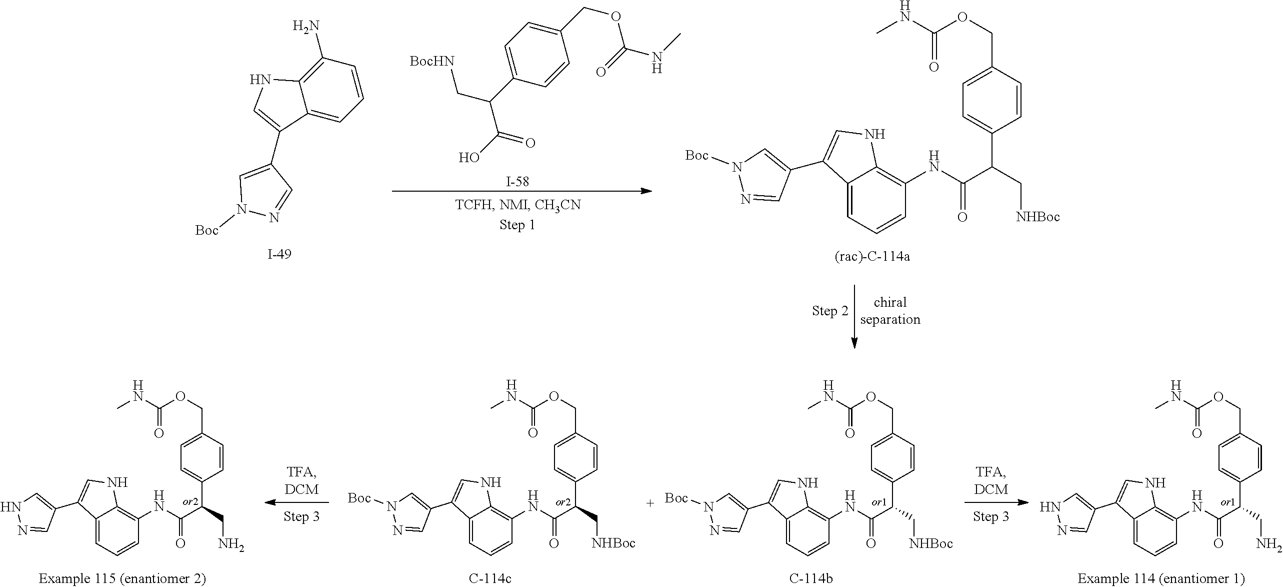

Step 1: Into a 20 mL vial were added acid I-58 (454.74 mg, 1.29 mmol, 1.1 equiv) and amine I-49 (350 mg, 1.17 mmol, 1 equiv) and TCFH (394.99 mg, 1.41 mmol, 1.2 equiv) and NMI (192.64 mg, 2.35 mmol, 2 equiv) and MeCN (5 mL) at room temperature. The resulting mixture was stirred for 2 h at room temperature under nitrogen atmosphere. The resulting mixture was concentrated under reduced pressure. The residue was purified by prep-TLC with CH₂Cl₂/methanol (25:1, v/v) to afford racemic amide C-114a (310 mg, 41.76%) as a yellow oil. LCMS: m/z (ESI), [M+H]⁺=633.40.

Step 2: The racemic amide C-114a (310 mg) was purified by prep-Chiral-HPLC with the following conditions (Column: CHIRALPAK IE, 2*25 cm, 5 um; Mobile Phase A: Hex (0.2% DEA), Mobile Phase B: EtOH:DCM, (1:1, v/v); Flow rate: 20 mL/min; Gradient: 30% B to 30% B in 31 min; wavelength: 220/254 nm; t_{R}−1=18.21 min; t_{R}−2=24.77 min; Sample Solvent: EtOH:DCM, (1:1, v/v); Injection Volume: 1.25 mL) to afford enantiomer C-114c (120 mg, 38.71%), SFC: t_{R}=4.743 min, ee=100%, and enantiomer C-114b (90 mg, 29.03%) as a yellow solid, SFC: t_{R}=6.999 min, ee=100%.

Step 3: Into a 100 mL round-bottom flask were added enantiomer C-114c (120 mg, 0.19 mmol, 1 equiv) and TFA (1 mL) and DCM (3 mL) at room temperature. The resulting mixture was stirred for 1 h at room temperature under air atmosphere. The resulting mixture was concentrated under reduced pressure. The residue was dissolved in DMF (2 mL). The mixture was basified to pH 9 with NH₃ (aq). The resulting mixture was stirred for 30 min at 0° C. The crude product (120 mg) was purified by prep-HPLC with the following conditions (Column: XBridge Shield RP18 OBD 30*150 mm, 5 um; Mobile Phase A: water (10 mmol/L NH₄HCO₃), Mobile Phase B: ACN; Flow rate: 60 mL/min; Gradient: 10% B to 35% B in 8 min; wavelength: 220 nm; t_{R}=7.80 min) to afford Example 115, enantiomer 2 (39.5 mg, 48.10%) as a white solid. LCMS: m/z (ESI), [M+H]⁺=433.20. ¹H NMR (CD₃OD, 400 MHZ) δ 2.71 (3H, s), 3.05 (1H, dd), 3.44 (1H, dd), 3.99 (1H, td), 5.09 (2H, s), 7.05 (2H, m), 7.45 (5H, m), 7.63 (1H, dt), 7.91 (2H, s). SFC: t_{R}=1.753 min, ee=92.58%.

Into a 100 mL round-bottom flask were added enantiomer C-114b (90 mg, 0.14 mmol, 1 equiv) and TFA (1 mL) and DCM (3 mL) at room temperature. The resulting mixture was stirred for 1 h at room temperature under air atmosphere. The resulting mixture was concentrated under reduced pressure. The residue was dissolved in DMF (2 mL). The mixture was basified to pH 9 with NH₃ (aq). The resulting mixture was stirred for 30 min at 0° C. The crude product (90 mg) was purified by prep-HPLC with the following conditions (Column: XBridge Prep OBD 30*150 mm, 5 um; Mobile Phase A: water (10 mmol/L NH₄HCO₃), Mobile Phase B: ACN; Flow rate: 60 mL/min; Gradient: 5% B to 35% B in 7 min; wavelength: 220 nm; t_{R}=8.00 min) to afford Example 114, enantiomer 1 (37.2 mg, 60.22%) as a white solid. LCMS: m/z (ESI), [M+H]⁺=433.10. ¹H NMR (CD₃OD, 400 MHz) δ 2.71 (3H, s), 3.04 (1H, dd), 3.44 (1H, dd), 3.98 (1H, dd), 5.09 (2H, s), 7.05 (2H, m), 7.40 (2H, d), 7.47 (3H, m), 7.64 (1H, m), 7.91 (2H, s). SFC: t_{R}=1.914 min, ee=98.90%.

### Example 116 (Enantiomer 1) and Example 117 (Enantiomer 2)

### Preparation of N-(3-(1H-pyrazol-4-yl)-1H-indol-7-yl)-2-(3-cyanophenyl)-3-(4-methylpiperazin-1-yl)propanamide

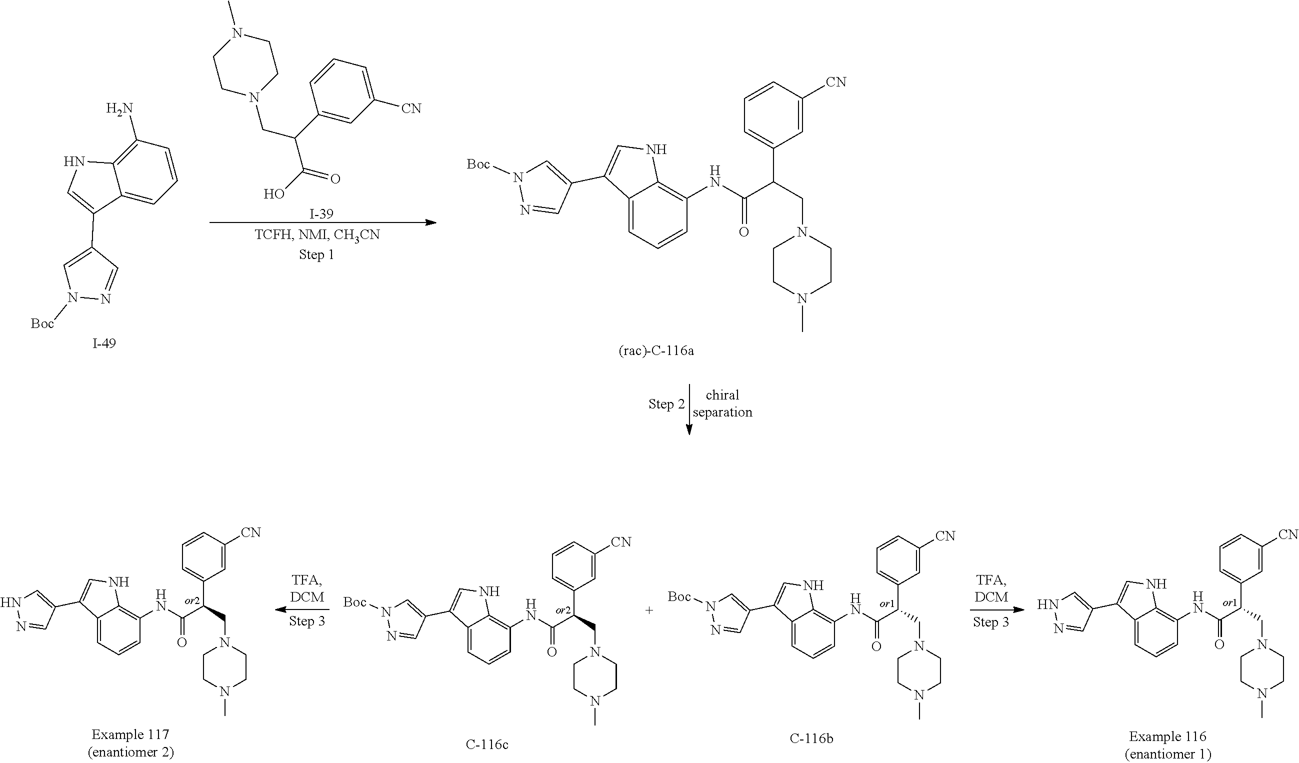

Step 1: To a stirred mixture of acid I-39 (200 mg, 0.37 mmol, 1 equiv) in CH₃CN (6.0 mL) was added NMI (141.86 mg, 1.10 mmol, 3 equiv), TCFH (278.22 mg, 0.73 mmol, 2 equiv) and amine I-49 (110.24 mg, 0.37 mmol, 1.01 equiv) in portions at room temperature under nitrogen atmosphere. After reaction, the resulting mixture was diluted with water (20 mL). The resulting mixture was extracted with ethyl acetate (3×20 mL). The combined organic layers were washed with water (3×10 mL), dried over anhydrous Na₂SO₄. After filtration, the filtrate was concentrated under reduced pressure. The residue was purified by reverse flash chromatography with the following conditions: column, C18 silica; mobile phase, methanol in water, 10% to 100% gradient in 10 min; detector, UV 254 nm and hold for 30 min at 90%. After evaporated we afford racemic amide C-116a (180 mg, 44.44%) as a yellow oil. LCMS m/z (ESI), [M+H]⁺=554.25.

Step 2: racemic amide C-116a (180 mg) was separated by prep-chiral HPLC with the following condition (Column: DZ-CHIRALPAK IE-3, 4.6*50 mm, 3.0 um; Mobile Phase A: Hex (0.2% DEA): (EtOH:DCM=1:1, v/v)=70:30 (v/v); Flow rate: 1 mL/min; Gradient: 0% B to 0% B; Injection Volume: 5 ul mL) to afford enantiomer C-116c (60 mg, 33.33%), SFC: t_{R}=4.527 min, ee=97.5%, and enantiomer C-116b (60 mg, 33.33%) as a white solid, SFC: t_{R}=5.122 min, ee=95.32%.

Step 3: A solution of enantiomer C-116c (55 mg, 0.099 mmol, 1 equiv) and TFA (0.5 mL) in DCM (2.0 mL) was stirred for 0.5 h at room temperature under nitrogen atmosphere. After reaction, the resulting mixture was concentrated under vacuum. The residue was dissolved in methanol (1.2 mL). The residue was basified to pH 9 with NH₃ aq (0.5 mL). The resulting mixture was purified by prep-HPLC with the following conditions (Column: XBridge Shield RP18 OBD 30*150 mm, 5 um; Mobile Phase A: water (10 mmol/L NH₄HCO₃), Mobile Phase B: ACN; Flow rate: 60 mL/min; Gradient: 20% B to 45% B in 8 min; wavelength: 220 nm; t_{R}=7.27 min) to afford Example 117, enantiomer 2 (13.6 mg, 30.22%) as a white solid. LCMS: m/z (ESI), [M+H]⁺=454.25. ¹H NMR (CD₃OD, 400 MHz) δ 2.31 (3H, s), 2.67 (9H, m), 3.40 (1H, m), 4.21 (1H, dd), 7.08 (2H, m), 7.51 (1H, s), 7.58 (1H, t), 7.68 (2H, m), 7.86 (4H, m). SFC: t_{R}=2.013 min, ee=96.46%.

A solution of enantiomer C-116b (55 mg, 0.099 mmol, 1 equiv) and trifluoroacetic acid (0.5 mL) in CH₂Cl₂ (2.0 mL) was stirred for 0.5 h at room temperature under nitrogen atmosphere. After cooling to room temperature, the resulting mixture was concentrated under vacuum. The residue was dissolved in methanol (1.2 mL). The residue was basified to pH 9 with NH₃ aq (0.5 mL). The resulting mixture was purified by prep-HPLC with the following conditions (Column: XBridge Shield RP18 OBD 30*150 mm, 5 um; Mobile Phase A: water (10 mmol/L NH₄HCO₃), Mobile Phase B: ACN; Flow rate: 60 mL/min; Gradient: 20% B to 45% B in 8 min; wavelength: 220 nm; t_{R}=7.27 min) to afford Example 116, enantiomer 1 (11.9 mg, 26.44%) as a white solid. LCMS: m/z (ESI), [M+H]⁺=454.25. ¹H NMR (CD₃OD, 400 MHZ) δ 2.31 (3H, s), 2.67 (9H, m), 3.40 (1H, dd), 4.21 (1H, dd), 7.09 (2H, m), 7.51 (1H, s), 7.58 (1H, t), 7.68 (2H, m), 7.86 (4H, m). SFC: t_{R}=2.232 min, ee=95.84%.

### Example 118 (Racemic)

### Preparation of N-(3-(1H-pyrazol-4-yl)-1H-indol-7-yl)-2-amino-2-(4-(hydroxymethyl)phenyl) acetamide

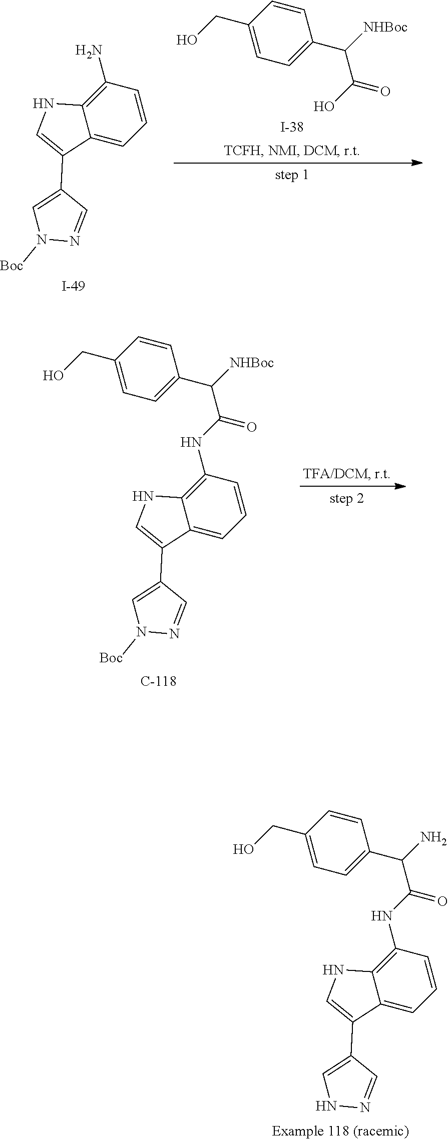

Step 1: To a solution of acid I-38 (300 mg, 1.07 mmol, 1 equiv) in DCM (20 mL) were added amine I-49 (340 mg, 1.14 mmol, 1 equiv), TCFH (360 mg, 1.28 mmol, 1.2 equiv) and NMI (140 mg, 1.70 mmol, 1.5 equiv) for 5 h at room temperature. The resulting mixture was concentrated under reduced pressure. The residue was purified by prep-HPLC with the following conditions (The mobile phase consisted of a mixture of solvent 0.1% NH₄HCO₃ in water and 0.1% NH₄OH in acetonitrile. A constant gradient from 70% aqueous/30% organic to 47% aqueous/53% organic mobile phase over the course of 8 minutes was utilized. The flow rate was constant at 60 mL/min.) to give 38 mg of racemic amide C-118 as a brown oil (6%). LCMS: m/z (ESI), [M+H]⁺=562.26. ¹H NMR (DMSO-d₆, 400 MHZ) δ 1.42 (9H, s), 1.63 (9H, s), 4.46 (2H, d), 5.18 (1H, t), 5.44 (1H, d), 7.06 (1H, t), 7.31 (3H, d), 7.42-7.68 (6H, m), 7.84 (1H, d), 8.27 (1H, s), 8.50 (1H, s), 10.05 (1H, d), 10.92 (1H, s).

Step 2: A solution of racemic amide C-118 (35 mg, 0.06 mmol, 1 equiv) and TFA (1 mL, 13.46 mmol) in DCM (5 mL) was stirred for 30 min at 0° C. under nitrogen atmosphere. The resulting mixture was concentrated under reduced pressure. The residue was neutralized to pH 7˜8 with sat. NaHCO₃ and extracted with CH₂Cl₂ (3×10 mL). The combined organic layers were dried over anhydrous Na₂SO₄. After filtration, the filtrate was concentrated under reduced pressure. The residue was purified by prep-HPLC with the following conditions (the mobile phase consisted of a mixture of solvent 0.1% NH₄HCO₃ in water and 0.1% NH₄OH in acetonitrile. A constant gradient from 70% aqueous/30% organic to 47% aqueous/53% organic mobile phase over the course of 8 minutes was utilized. The flow rate was constant at 60 mL/min) to give 9 mg of Example 118, racemic as a yellow solid (36%). LCMS: m/z (ESI), [M+H]⁺=362.15. ¹H NMR (DMSO-d₆, 400 MHZ) δ 4.46 (2H, d), 4.63 (1H, s), 5.14 (1H, t), 6.83 (1H, t), 7.29 (2H, d), 7.42-7.52 (3H, m), 7.52-7.62 (2H, m), 7.84 (1H, s), 8.06 (1H, s), 10.83 (1H, s), 12.81 (1H, s).

### Example 119 (Enantiomer 1) and Example 120 (Enantiomer 2)

### Preparation of tert-butyl (2-((3-(1H-pyrazol-4-yl)-1H-indol-7-yl)amino)-1-(5-methoxypyridin-3-yl)-2-oxoethyl) carbamate

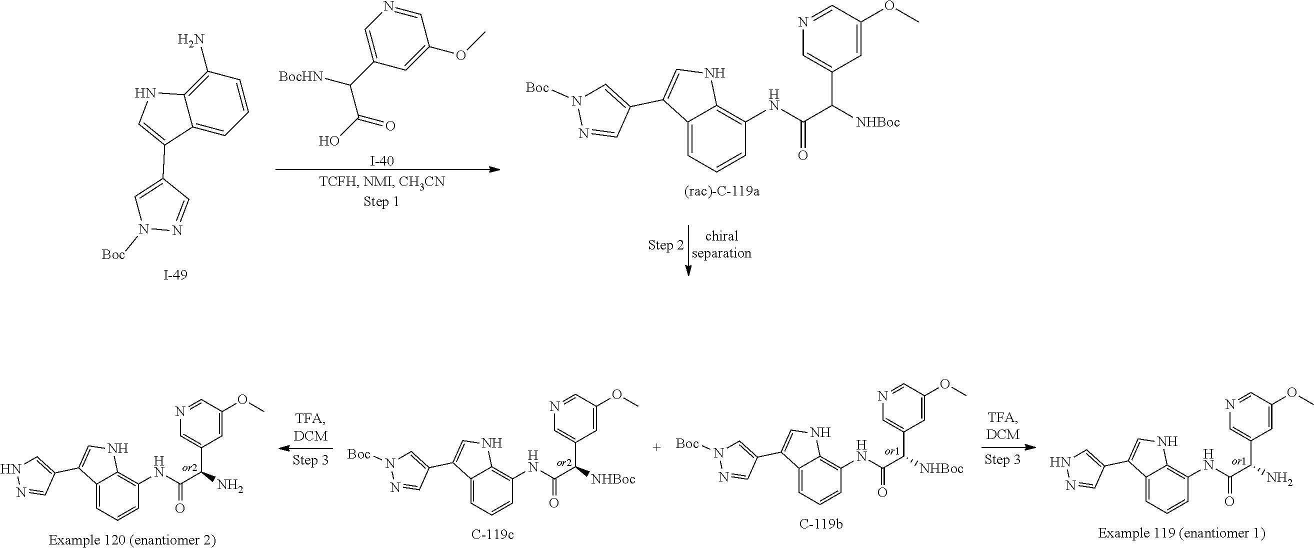

Step 1: Into a 50 mL round-bottom flask were added acid I-40 (250 mg, 0.89 mmol, 1 equiv), amine I-49 (264.21 mg, 0.89 mmol, 1 equiv), TCFH (496.96 mg, 1.77 mmol, 2 equiv), NMI (145.43 mg, 1.77 mmol, 2 equiv) and MeCN (5 mL) at room temperature. The resulting mixture was stirred for 6 h at room temperature. The residue was purified by reverse flash chromatography with the following conditions: column, C18 silica; mobile phase, methanol in water, 61.5% gradient in 35 min; detector, UV 254 nm. This resulted in racemic amide C-119a (259 mg, 44.18%) as a yellow solid. LCMS: m/z (ESI), [M+H]⁺=563.35.

Step 2: The racemic amide C-119a (259 mg, 44.18%) was purified by prep-chiral HPLC with the following conditions (Column: CHIRALPAK IG, 2*25 cm, 5 um; Mobile Phase A: Hex (0.2% DEA), Mobile Phase B: EtOH:DCM (1:1, v/v); Flow rate: 20 mL/min; Gradient: 50% B to 50% B in 9.5 min; wavelength: 220/254 nm; t_{R}−1=5.32 min; t_{R}−2=7.56 min; Sample Solvent: EtOH:DCM (1:1, v/v); Injection Volume: 0.5 mL) to afford enantiomer C-119c (120.5 mg, 46.52%), SFC: t_{R}=1.195 min, ee=95.32%, and enantiomer C-119b (120.1 mg, 46.3%) as a white solid, SFC: t_{R}=1.711 min, ee=95.02%.

Step 3: Into a 50 mL round-bottom flask were added enantiomer C-119c (120 mg, 0.21 mmol, 1 equiv), TFA (1 mL) and DCM (3 mL) at room temperature. The resulting mixture was stirred for 1 h at room temperature. The mixture was concentrated under reduced pressure, dissolved in methanol, basified to pH 9 with NH₃ (aq) at 0° C. The crude product (90 mg) was purified by prep-HPLC with the following conditions (Column: XBridge Prep OBD 30*150 mm, 5 um; Mobile Phase A: water (10 mmol/L NH₄HCO₃), Mobile Phase B: ACN; Flow rate: 60 mL/min; Gradient: 7% B to 37% B in 7 min; wavelength: 220 nm; t_{R}=6.73 min) to afford Example 120, enantiomer 2 (26.3 mg, 33.92%) as a white solid. LCMS: m/z (ESI), [M+H]⁺=363.20. ¹H NMR (CD₃OD, 400 MHZ) δ 3.93 (3H, s), 4.83 (1H, s), 7.04-7.16 (2H, m), 7.48 (1H, s), 7.63-7.70 (2H, m), 7.92 (2H, s), 8.23 (1H, d), 8.36 (1H, d). SFC: t_{R}=2.687 min, ee=97.22%.

Into a 50 mL round-bottom flask were added enantiomer C-119b (120 mg, 0.21 mmol, 1 equiv), TFA (1 mL) and DCM (3 mL) at room temperature. The resulting mixture was stirred for 1 h at room temperature. The mixture was concentrated under reduced pressure, dissolved in methanol (2 mL), basified to pH 9 with NH₃ (aq) at 0° C. The crude product (90 mg) was purified by prep-HPLC with the following conditions (Column: XBridge Shield RP18 OBD 30*150 mm, 5 um; Mobile Phase A: water (10 mmol/L NH₄HCO₃), Mobile Phase B: ACN; Flow rate: 60 mL/min; Gradient: 9% B to 34% B in 8 min; wavelength: 220 nm; t_{R}=7.45 min) to afford Example 119, enantiomer 1 (23.4 mg, 30.18%) as a white solid. LCMS: m/z (ESI), [M+H]⁺=363.15. ¹H NMR (CD₃OD, 400 MHz) δ 3.93 (3H, s), 4.83 (1H, s), 7.04-7.16 (2H, m), 7.48 (1H, s), 7.63-7.70 (2H, m), 7.92 (2H, s), 8.23 (1H, d), 8.36 (1H, d). SFC: t_{R}=2.515, ce=92.72%.

### Example 121

### Preparation of (2R)-3-methoxy-2-(4-methylpiperazin-1-yl)-N-[3-(1H-pyrazol-4-yl)-1H-indol-7-yl]propanamide

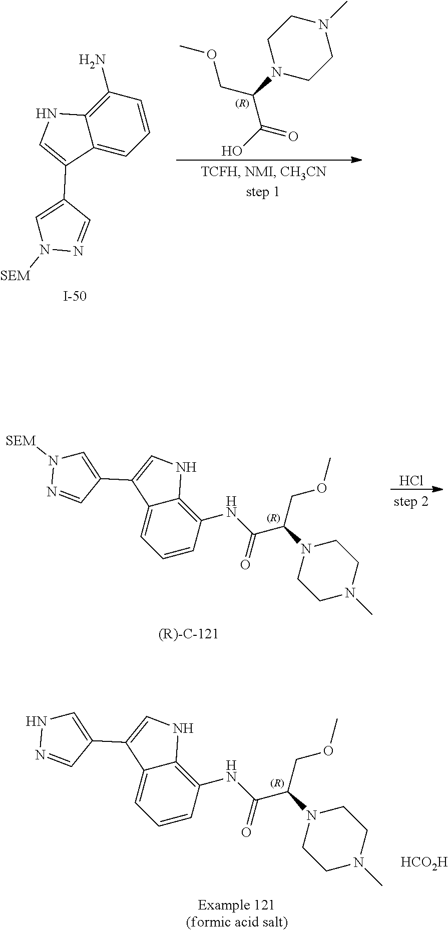

Step 1: Into a 8 mL vial were added 3-(1-{[2-(trimethylsilyl) ethoxy]methyl}pyrazol-4-yl)-1H-indol-7-amine I-50 (200 mg, 0.61 mmol, 1 equiv), TCFH (256.24 mg, 0.91 mmol, 1.5 equiv), NMI (199.96 mg, 2.44 mmol, 4 equiv) and (2R)-3-methoxy-2-(4-methylpiperazin-1-yl)propanoic acid (147.77 mg, 0.73 mmol, 1.2 equiv) in ACN (2 mL) at room temperature. The resulting mixture was stirred for overnight at room temperature under nitrogen atmosphere. The resulting mixture was extracted with EA (2×10 mL). The combined organic layers were concentrated under reduced pressure. The residue was purified by prep-TLC with CH₂Cl₂/methanol (15:1, v/v) to afford (2R)-3-methoxy-2-(4-methylpiperazin-1-yl)-N-[3-(1-{[2-(trimethylsilyl) ethoxy]methyl}pyrazol-4-yl)-1H-indol-7-yl]propanamide C-121 (140 mg, 44.85%) as a purple solid. LCMS: m/z (ESI), [M+H]⁺=513.15.

Step 2: Into a 8 mL vial were added (2R)-3-methoxy-2-(4-methylpiperazin-1-yl)-N-[3-(1-{[2-(trimethylsilyl) ethoxy]methyl}pyrazol-4-yl)-1H-indol-7-yl]propanamide C-121 (90 mg, 0.18 mmol, 1 equiv) and HCl (gas) in 1,4-dioxane (1.5 mL) at room temperature. The resulting mixture was stirred for 1 h at room temperature. The residue was purified by reverse flash chromatography with the following conditions: column, C18 silica; mobile phase, methanol in water, 0% to 100% gradient in 40 min; detector, UV 254 nm. The crude product was purified by prep-HPLC with the following conditions (Column: Xselect CSH C18 OBD 30*150 mm 5 um; Mobile Phase A: water (0.1% FA), Mobile Phase B: ACN; Flow rate: 60 mL/min; Gradient: 5% B to 15% B in 7 min; wavelength: 254; 220 nm; t_{R}=5.98 min) to afford (2R)-3-methoxy-2-(4-methylpiperazin-1-yl)-N-[3-(1H-pyrazol-4-yl)-1H-indol-7-yl]propanamide Example 121 (1.2 mg, 1.79%) as a white solid. LCMS: m/z (ESI), [M+H]⁺=383.15. ¹H NMR (CD₃OD, 400 MHz) δ 2.73 (3H, d), 3.08 (8H, d), 3.45 (3H, s), 3.67 (1H, t), 3.86-3.92 (1H, m), 3.95 (1H, dd), 7.11 (1H, t), 7.15-7.21 (1H, m), 7.48 (1H, s), 7.7 (1H, d), 7.93 (2H, s), 8.46 (1H, s).

### Example 122 (Racemic)

### Preparation of N-(3-(1H-pyrazol-4-yl)-1H-indol-7-yl)-2-amino-2-cyclohexylacetamide

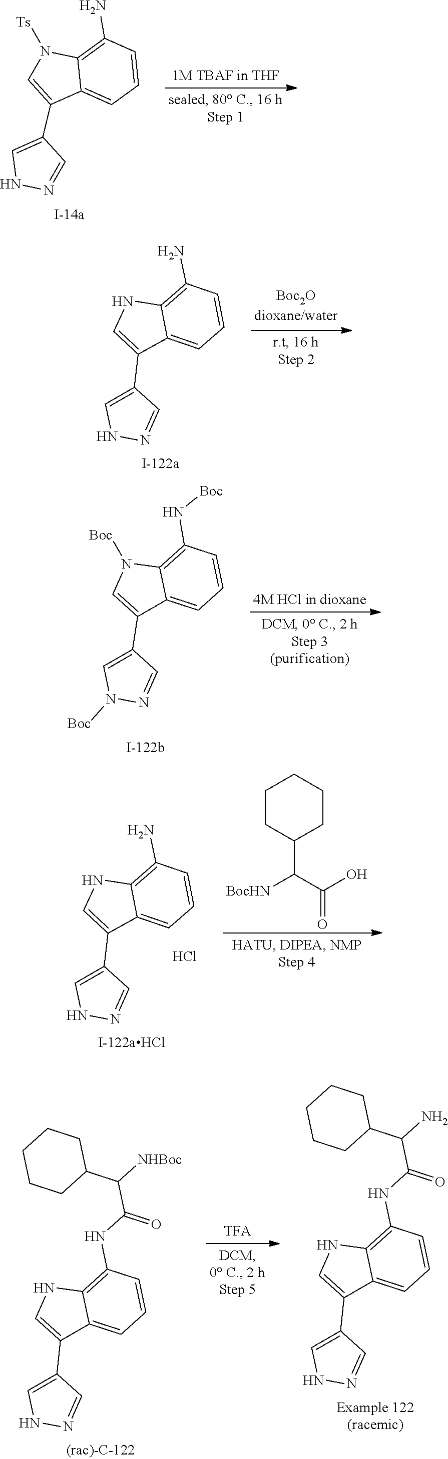

Step 1 and Step 2: A solution of amine I-14a (2.73 g, 7.75 mmol) was dissolved into 1 M TBAF (10.47 g, 40.05 mmol, 11.60 mL). The reaction was sealed and heated at 80° C. for 16 hr. The reaction solution was evaporated under reduced pressure. LCMS: t_{R}=0.67 min in 3 min chromatography (3 min-5-95% MeCN in water (0.02% TFA), Waters Acquity UPLC BEH C18 1.7 μm, 2.1*50 mm, 40° C.), MS (ESI) m/z=199.5 [M+1]⁺. The residue was dissolved in dioxane (25 mL)/water (25 mL). Then added tert-butoxycarbonyl tert-butyl carbonate (10.14 g, 46.48 mmol, 10.67 mL) and sodium hydrogen carbonate (6.51 g, 77.47 mmol, 3.01 mL). The resulting mixture was stirred at room temperature for 16 h. Then the organic solution was evaporated under reduced pressure. Extracted with ethyl acetate (30 mL×3), the combined organic was washed with water (50 mL×3) and brine (50 mL×1), dried over anhydrous sodium sulfate, filtered and evaporated under reduced pressure. The residue was purified by silica flash chromatography column, elution gradient from 0 to 30% ethyl acetate in petroleum ether. Pure fractions were evaporated to dryness to afford tert-butyl 7-(tert-butoxycarbonylamino)-3-(1-tert-butoxycarbonylpyrazol-4-yl) indole-1-carboxylate I-122b (800 mg, 21% yield) brown solid. LCMS: t_{R}=2.14 min in 3 min chromatography (3 min-5-95% MeCN in water (0.02% TFA), Waters Acquity UPLC BEH C18 1.7 μm, 2.1*50 mm, 40° C.), MS (ESI) m/z=443.7 [M−55]⁺.

Step 3: A solution of tert-butyl 7-(tert-butoxycarbonylamino)-3-(1-tert-butoxycarbonylpyrazol-4-yl) indole-1-carboxylate I-122b (800 mg, 1.60 mmol) in DCM (20 mL) was stirred and cooled at 0° C. by ice/water bath. Then added 4M HCl (1.76 g, 48.14 mmol) in dioxane (12 mL) dropwise into the reaction. The reaction mixture was stirred at this temperature for 2 h. The solution was evaporated under reduced pressure to afford 3-(1H-pyrazol-4-yl)-1H-indol-7-amine I-122a (519 mg, 94% yield, HCl salt) as light brown solid. LCMS: t_{R}=2.31 min in 7 min chromatography (3 min-5-95% MeCN in water (0.02% TFA), Waters Acquity UPLC BEH C18 1.7 μm, 2.1*50 mm, 40° C.), MS (ESI) m/z=199.6 [M+1]⁺.

Step 4: To a stirred mixture of 3-(1H-pyrazol-4-yl)-1H-indol-7-amine I-122a (75 mg, 217.98 μmol, HCl salt), 2-(tert-butoxycarbonylamino)-2-cyclohexyl-acetic acid (20 mg, 77.72 μmol) and N-ethyl-N-isopropyl-propan-2-amine (100.45 mg, 777.23 μmol, 135.38 μL) in NMP (1.50 mL) was stirred and cooled at 0° C. by ice/water bath. Then HATU (35.65 mg, 93.76 μmol) was added in one portion at this temperature. The resulting mixture was stirred and warmed slowly to room temperature for 2 h. The reaction was purified by C18 flash chromatography column, elution gradient from 0 to 80% MeCN in water (6 mmol/L NH₄HCO₃). Pure fractions were lyophilized to dryness to afford racemic amide C-122 (20 mg, 59% yield) as brown solid. LCMS: t_{R}=2.08 min in 3 min chromatography (3 min-5-95% MeCN in water (0.02% TFA), Waters Acquity UPLC BEH C18 1.7 μm, 2.1*50 mm, 40° C.), MS (ESI) m/z=438.4 [M+H]⁺.

Step 5: To a solution of racemic amide C-122 (20 mg, 45.71 μmol) in DCM (1.00 mL) was stirred and cooled at 0° C. by ice/water bath. Then added TFA (521.21 mg, 4.57 mmol, 352.17 μL) dropwise into the reaction. The resulting mixture was stirred at 0° C. for 2 h. The reaction mixture was concentrated under reduced pressure. The residue was purified by C18-flash chromatography, elution gradient from 0% to 30% MeCN in water (0.02% TFA). Pure fractions were lyophilized to dryness to afford Example 122, racemic (4.56 mg, 22% yield, TFA) as brown solid. LCMS: t_{R}=1.27 min in 3 min chromatography (3 min-5-95% MeCN in water (0.02% TFA), Waters Acquity UPLC BEH C18 1.7 μm, 2.1*50 mm, 40° C.), MS (ESI) m/z=338.4 [M+H]⁺. ¹H NMR: (400 MHZ, DMSO-d₆) δ 10.72 (s, 1H), 10.35 (s, 1H), 8.26 (s, 3H), 7.97 (s, 2H), 7.66 (d, J=7.9 Hz, 1H), 7.63 (d, J=2.4 Hz, 1H), 7.50 (d, J=7.5 Hz, 1H), 7.06 (t, J=7.8 Hz, 1H), 3.85 (d, J=7.1 Hz, 1H), 1.67-1.55 (m, 5H), 1.13-1.00 (m, 6H).

### Example 123 (Racemic), Example 124 (Enantiomer 1), and Example 125 (Enantiomer 2)

### Preparation of 3-(1-((3-(1H-pyrazol-4-yl)-1H-indol-7-yl)amino)-3-amino-1-oxopropan-2-yl)benzamide

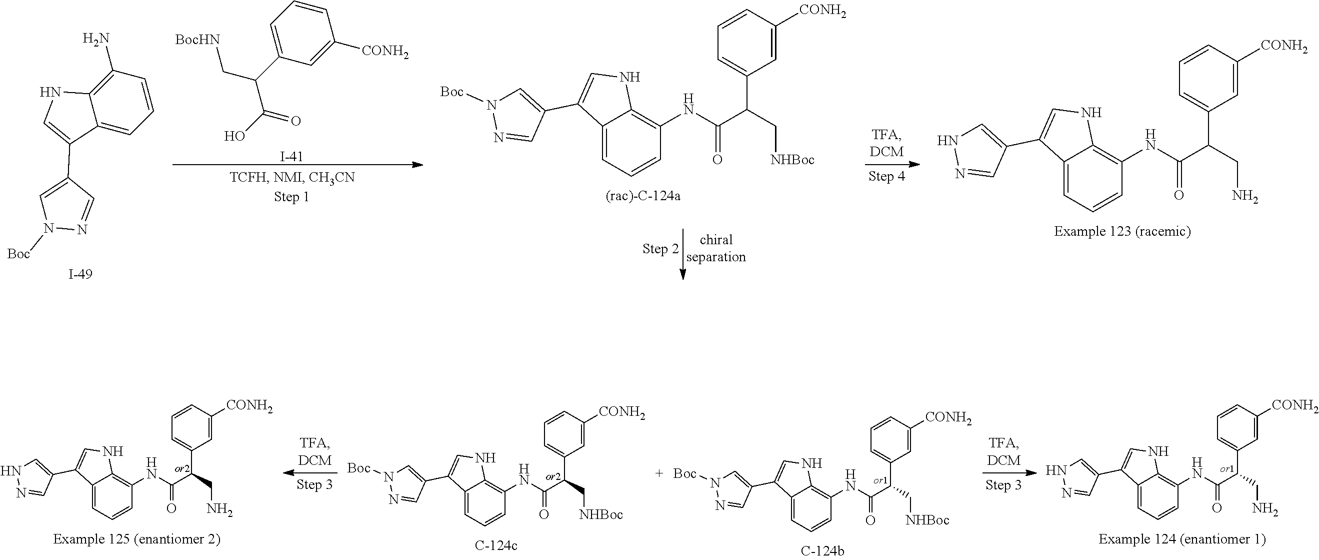

Step 1: To a stirred solution of acid I-41 (330 mg, 1.07 mmol, 1 equiv) and amine I-49 (319.31 mg, 1.07 mmol, 1 equiv) and TCFH (450.44 mg, 1.61 mmol, 1.5 equiv) in MeCN (3 mL) were added NMI (307.56 mg, 3.75 mmol, 3.5 equiv) dropwise at room temperature under air atmosphere. The resulting mixture was stirred for 2 h at room temperature under air atmosphere. The resulting mixture was concentrated under reduced pressure. The residue was purified by reverse flash chromatography with the following conditions: column, C18 silica; mobile phase, MeCN in water, 10% to 50% gradient in 50 min; detector, UV 254 nm. This resulted in racemic amide C-124a (489 mg, 77.62%) as a white solid. LCMS: m/z (ESI), [M+H]⁺=589.35. ¹H NMR (CD₃OD, 400 MHZ) δ 1.43 (9H, s), 1.71 (9H, s), 3.54 (1H, m), 3.83 (1H, m), 4.18 (1H, m), 7.12 (2H, m), 7.50 (1H, t), 7.65 (2H, d), 7.71 (1H, d), 7.82 (1H, d), 8.02 (1H, t), 8.16 (1H, s), 8.43 (1H, s).

Step 2. The racemic amide C-124a (360 mg) was purified by prep-chiral HPLC with the following conditions (Column: CHIRALPAK IC, 2*25 cm, 5 um; Mobile Phase A: Hex (0.2% DEA), Mobile Phase B: EtOH:DCM (1:1, v/v); Flow rate: 20 mL/min; Gradient: 30% B to 30% B in 18 min; wavelength: 220/254 nm; t_{R}−1=10.22 min; t_{R}−2=14.67 min; Sample Solvent: EtOH:DCM (1:1, v/v); Injection Volume: 0.5 mL) to afford enantiomer C-124c (100 mg, 27.78%) as a white solid, SFC: t_{R}=1.404 min, ee=100%, and enantiomer C-124b (210 mg, 58.33%) as a white solid, SFC: t_{R}=1.819 min, ee=100%.

Step 3: A solution of enantiomer C-124c (100 mg, 0.17 mmol, 1 equiv) and TFA (1 mL) in DCM (3 mL) was stirred for 1 h at room temperature under air atmosphere. The resulting mixture was concentrated under reduced pressure. The resulting mixture was diluted with DMF (0.5 mL). The residue was basified to pH 9 with NH₃ (aq) at 0° C. The crude product (80 mg) was purified by prep-HPLC with the following conditions (Column: XBridge Prep OBD 30*150 mm, 5 um; Mobile Phase A: water (10 mmol/L NH₄HCO₃), Mobile Phase B: ACN; Flow rate: 60 mL/min; Gradient: 2% B to 30% B in 7 min; wavelength: 220 nm; t_{R}=7.18 min) to afford Example 125, enantiomer 2 (29.7 mg, 45.01%) as a white solid. LCMS: m/z (ESI), [M+H]⁺=389.15. ¹H NMR (CD₃OD, 400 MHz) δ 3.04 (1H, m), 3.44 (1H, m), 3.99 (1H, m), 7.06 (2H, m), 7.47 (2H, m), 7.63 (2H, m), 7.80 (1H, d), 7.89 (2H, s), 7.98 (1H, d). SFC: t_{R}=0.650, ee=100%.

A solution of enantiomer C-124b (210 mg, 0.36 mmol, 1 equiv) and TFA (1 mL) in DCM (3 mL) was stirred for 1 h at room temperature under air atmosphere. The resulting mixture was concentrated under reduced pressure. The resulting mixture was diluted with DMF (0.5 mL). The residue was basified to pH 9 with NH₃ (aq). The crude product (100 mg) was purified by prep-HPLC with the following conditions (Column: XBridge Shield RP18 OBD 30*150 mm, 5 um; Mobile Phase A: water (10 mmol/L NH₄HCO₃), Mobile Phase B: ACN; Flow rate: 60 mL/min; Gradient: 3% B to 30% B in 8 min; wavelength: 220 nm; t_{R}=7.83 min) to afford Example 124, enantiomer 1 (70.1 mg, 50.59%) as a white solid. LCMS: m/z (ESI), [M+H]⁺=389.15. ¹H NMR (CD₃OD, 400 MHz) δ 3.04 (1H, m), 3.44 (1H, m), 3.99 (1H, m), 7.06 (2H, m), 7.47 (2H, m), 7.63 (2H, m), 7.80 (1H, d), 7.89 (2H, s), 7.98 (1H, d). SFC: t_{R}=1.140 min, ee=100%.

Step 4: A solution of tert-butyl 4-(7-{3-[(tert-butoxycarbonyl)amino]-2-(3-carbamoylphenyl)propanamido}-1H-indol-3-yl) pyrazole-1-carboxylate (100 mg, 0.17 mmol, 1 equiv) and TFA (1 mL) in DCM (2 mL) was stirred for 2 h at room temperature under air atmosphere. The resulting mixture was concentrated under reduced pressure. The residue was basified to pH 9 with NH₃ (aq). The crude product (50 mg) was purified by prep-HPLC with the following conditions (Column: XBridge Prep OBD 30*150 mm, 5 um; Mobile Phase A: water (10 mmol/L NH₄HCO₃), Mobile Phase B: ACN; Flow rate: 60 mL/min; Gradient: 5% B to 25% B in 7 min; wavelength: 220 nm; t_{R}=6.72 min) to afford 3-(1-((3-(1H-pyrazol-4-yl)-1H-indol-7-yl)amino)-3-amino-1-oxopropan-2-yl)benzamide (35.5 mg, 53.80%) as a white solid. LCMS: m/z (ESI), [M+H]⁺=389.25.

### Example 126

### Preparation of (R)-N-(3-(1H-pyrazol-4-yl)-1H-indol-7-yl)-2-amino-3-phenylpropanamide

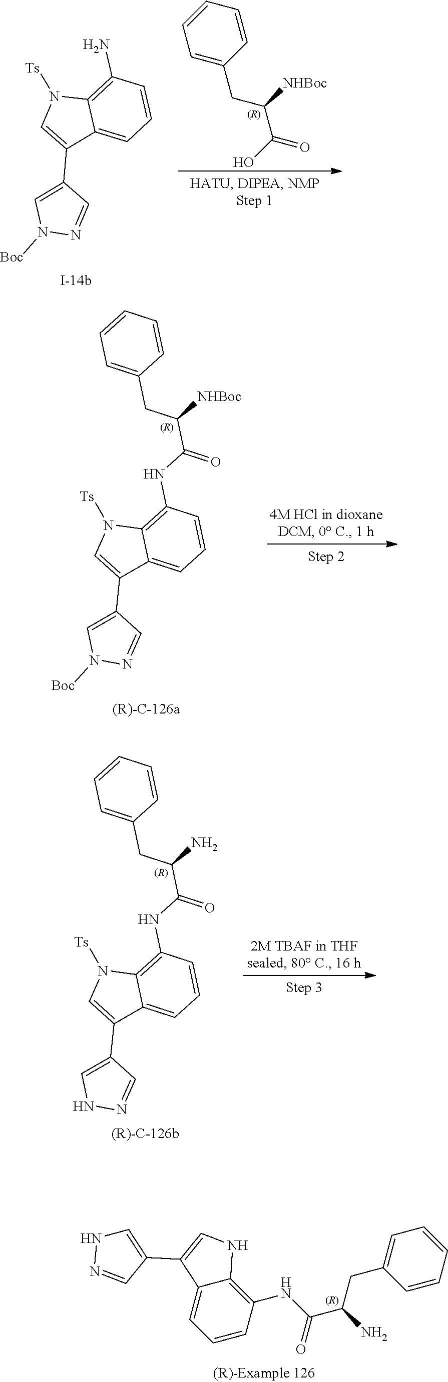

Step 1: A solution of tert-butyl 4-[7-amino-1-(p-tolylsulfonyl) indol-3-yl]pyrazole-1-carboxylate I-14b (142 mg, 313.79 μmol, TFA), (2R)-2-(tert-butoxycarbonylamino)-3-phenyl-propanoic acid (249.75 mg, 941.38 μmol) and DIPEA (243.33 mg, 1.88 mmol, 327.94 μL) in NMP (2.00 mL) was stirred and cooled at 0° C. by ice/water bath for 15 min. Then HATU (395.82 mg, 1.04 mmol) was added into the reaction at this temperature. The resulting suspension was stirred and warmed slowly to room temperature for 16 h. Then the reaction mixture was poured into water (50 mL), and extracted with ethyl acetate (30 mL×3). The combined organic layers were washed with brine (60 mL×1), dried over sodium sulfate, filtered, and the filtrate was concentrated under reduced pressure. The residue was purified by silica flash chromatography column, elution gradient from 15% to 40% ethyl acetate in petroleum ether. Pure fractions were evaporated to dryness to afford racemic amide C-126a (25 mg, 11% yield) as white solid. LCMS: t_{R}=2.23 min in 3 min chromatography (3 min-5-95% MeCN in water (0.02% TFA), Waters Acquity UPLC BEH C18 1.7 μm, 2.1*50 mm, 40° C.), MS (ESI) m/z=700.5 [M+H]⁺.

Step 2: To a solution of racemic amide C-126a (25 mg, 35.21 μmol) in DCM (2 mL) was stirred and cooled at 0° C. Then added 4 M HCl (128.38 mg, 3.52 mmol) dropwise into the reaction. The resulting mixture was stirred and warmed slowly to room temperature for 1 h. Then the organic solution was evaporated under reduced pressure to afford (2R)-2-amino-3-phenyl-N-[1-(p-tolylsulfonyl)-3-(1H-pyrazol-4-yl) indol-7-yl]propanamide C-126b (20 mg, 97% yield, HCl salt) as brown oil. LCMS: t_{R}=1.45 min in 3 min chromatography (3 min-5-95% MeCN in water (0.02% TFA), Waters Acquity UPLC BEH C18 1.7 μm, 2.1*50 mm, 40° C.), MS (ESI) m/z=500.4 [M+H]⁺.

Step 3: A solution of (2R)-2-amino-3-phenyl-N-[1-(p-tolylsulfonyl)-3-(1H-pyrazol-4-yl) indol-7-yl]propanamide C-126b (20 mg, 19.17 μmol, HCl salt) was dissolved into 1 M TBAF in THF (125.32 mg, 479.30 μmol, 0.48 mL). The reaction was sealed and heated at 80° C. for 16 h. The reaction solution was evaporated under reduced pressure. The residue was purified by C18-flash chromatography column, elution gradient from 0 to 30% CH₃CN in water (0.02% TFA). Pure fractions were evaporated to dryness to afford (2R)-2-amino-3-phenyl-N-[3-(1H-pyrazol-4-yl)-1H-indol-7-yl]propanamide Example 126 (3.0 mg, 45% yield) brown solid. LCMS: t_{R}=1.19 min in 3 min chromatography (3 min-5-95% MeCN in water (0.02% TFA), Waters Acquity UPLC BEH C18 1.7 μm, 2.1*50 mm, 40° C.), MS (ESI) m/z=346.3 [M+H]⁺. ¹H NMR: (400 MHZ, DMSO-d₆) δ 10.68 (s, 1H), 10.34 (s, 1H), 8.34 (s, 2H), 7.97 (s, 1H), 7.66 (d, J=8.3 Hz, 1H), 7.62 (d, J=2.4 Hz, 1H), 7.36 (d, J=4.4 Hz, 4H), 7.32-7.27 (m, 2H), 7.20 (s, 1H), 7.05 (t, J=7.8 Hz, 1H), 6.66 (s, 1H), 5.32 (t, J=4.5 Hz, 1H), 3.25-3.10 (m, 2H).

### Example 127 (Racemic)

### Preparation of Methyl 2-(4-bromophenyl)-3-[(tert-butoxycarbonyl)amino]propanoate

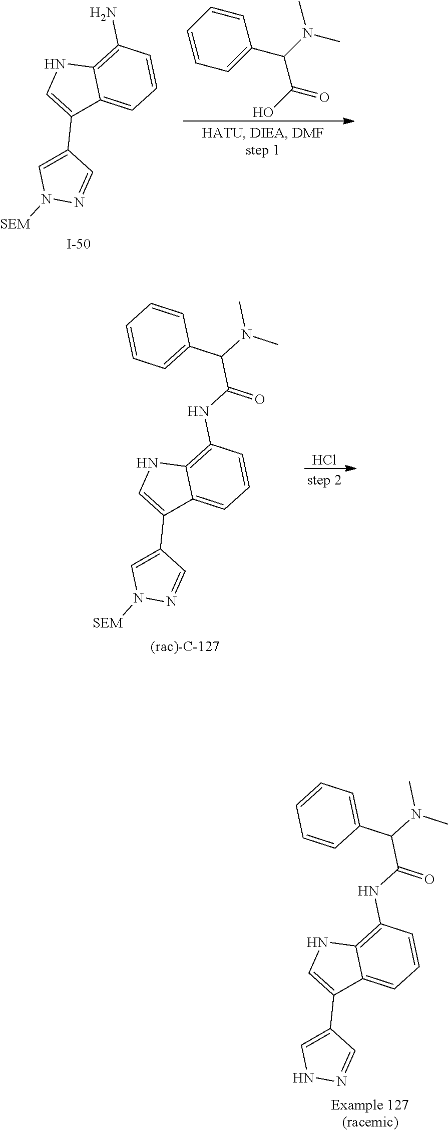

Step 1: A mixture of amine I-50 (100 mg, 0.30 mmol, 1 equiv), 2-(dimethylamino)-2-phenylacetic acid (60.01 mg, 0.33 mmol, 1.1 equiv), HATU (231.50 mg, 0.61 mmol, 2 equiv) and DIEA (118.04 mg, 0.91 mmol, 3 equiv) in DMF (3 mL) was stirred for 2 h at room temperature under nitrogen atmosphere. The residue was purified by reverse flash chromatography with the following conditions: column, C18 silica; mobile phase, MeCN in water (0.1% NH₃·H₂O), 10% to 50% gradient in 10 min; detector, UV 254 nm to afford racemic amide C-127 (100 mg, 67.08%) as a white solid. LCMS: m/z (ESI), [M−^{t}Bu+H]⁺=490.30. ¹H NMR (DMSO-d₆, 400 MHZ) δ 0.05 (9H, d), δ 0.86 (2H, m), 2.26 (6H, s), 3.59 (2H, dd), 4.02 (1H, s), 5.44 (2H, s), 7.01 (1H, t), 7.33 (1H, m), 7.39 (2H, m), 7.58 (5H, m), 7.89 (1H, d), 8.27 (1H, d), 9.92 (1H, s), 10.84 (1H, s).

Step 2: A solution of racemic amide C-127 (77 mg, 0.16 mmol, 1 equiv) was added HCl (gas) in 1,4-dioxane (10 mL) at 25° C. The mixture was stirred for 12 h at 40° C. The resulting mixture was concentrated under reduced pressure. The crude product (50 mg) was purified by prep-HPLC with the following conditions (Column: XBridge Prep OBD C18 30*150 mm, 5 um; Mobile Phase A: water (10 mmol/L NH₄HCO₃), Mobile Phase B: ACN; Flow rate: 60 mL/min; Gradient: 12% B to 37% B in 8 min; wavelength: 254 nm; t_{R}=7.33 min) to afford Example 127, racemic (1.5 mg, 2.56%) as a white solid. LCMS: m/z (ESI), [M+H]⁺=360.05. ¹H NMR (DMSO-d₆, 400 MHz) δ 2.26 (6H, s), 4.03 (1H, d), 6.99 (1H, t), 7.33 (1H, m), 7.39 (2H, dd), 7.52 (1H, d), 7.58 (4H, dt), 8.05 (2H, d), 9.93 (1H, s), 10.81 (1H, s), 12.82 (1H, s).

### Example 128

### Preparation of (2S)-N-[3-(1H-pyrazol-4-yl)-1H-indol-7-yl]pyrrolidine-2-carboxamide

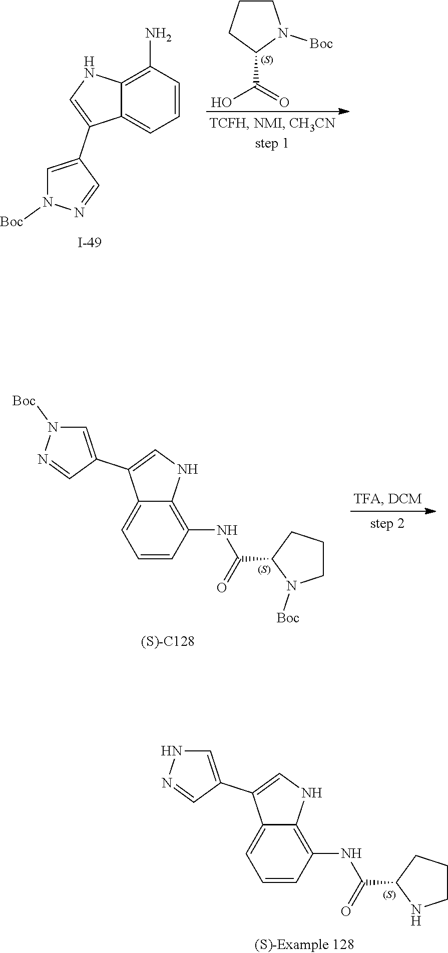

Step 1: Into a 20 mL vial were added amine I-49 (150 mg, 0.50 mmol, 1 equiv) and (2S)-1-(tert-butoxycarbonyl) pyrrolidine-2-carboxylic acid (129.87 mg, 0.60 mmol, 1.2 equiv) and TCFH (169.28 mg, 0.60 mmol, 1.2 equiv) and NMI (165.12 mg, 2.01 mmol, 4 equiv) and MeCN (5 mL) at room temperature. The resulting mixture was stirred for 1 h at room temperature under nitrogen atmosphere. The resulting mixture was concentrated under reduced pressure. The resulting mixture was diluted with water (40 mL). The aqueous layer was extracted with CH₂Cl₂ (3×40 mL). The combined organic layer was dried over anhydrous Na₂SO₄, filtered and evaporated to afford a crude solid. The residue was purified by prep-TLC with CH₂Cl₂/methanol (37:1, v/v) to afford tert-butyl 4-{7-[(2S)-1-(tert-butoxycarbonyl) pyrrolidine-2-amido]-1H-indol-3-yl}pyrazole-1-carboxylate C-128 (240 mg, 96.32%) as a yellow oil. LCMS: m/z (ESI), [M+H−^{t}Bu]⁺=440.20.

Step 2: Into a 50 mL vial were added tert-butyl 4-{7-[(2S)-1-(tert-butoxycarbonyl) pyrrolidine-2-amido]-1H-indol-3-yl}pyrazole-1-carboxylate C-128 (240 mg, 0.48 mmol, 1 equiv) and TFA (1 mL) and DCM (3 mL) at room temperature. The resulting mixture was stirred for 1 h at room temperature under air atmosphere. The resulting mixture was concentrated under reduced pressure. The residue was dissolved in DMF (2 mL). The mixture was basified to pH 9 with NH₃ aq. The resulting mixture was stirred for 30 min at 0° C. under air atmosphere. The crude product (150 mg) was purified by prep-HPLC with the following conditions (Column: XBridge Shield RP18 OBD 30*150 mm, 5 um; Mobile Phase A: water (50 mmol/L NH₄HCO₃), Mobile Phase B: ACN; Flow rate: 60 mL/min; Gradient: 5% B to 30% B in 7 min; wavelength: 220 nm; t_{R}=6.35 min) to afford (2S)-N-[3-(1H-pyrazol-4-yl)-1H-indol-7-yl]pyrrolidine-2-carboxamide Example 128 (92.8 mg, 64.11%) as a white solid. LCMS: m/z (ESI), [M+H]⁺=296.25. ¹H NMR (DMSO-d₆, 400 MHz) δ 1.73 (2H, m), 2.08 (2H, d), 2.93 (1H, m), 3.30 (1H, d), 4.35 (1H, s), 7.01 (1H, q), 7.51 (3H, m), 7.94 (2H, s). SFC: t_{R}=2.070 min, ee=99.56%.

### Example 129 (Racemic)

### Preparation of 2-cyclohexyl-2-(4-methylpiperazin-1-yl)-N-[3-(1H-pyrazol-4-yl)-1H-indol-7-yl]acetamide

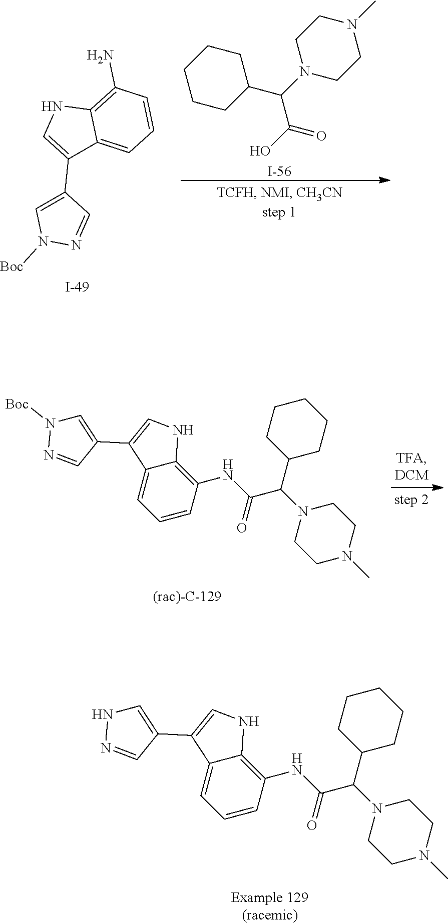

Step 1: Into a 20 mL vial were added amine I-49 (150 mg, 0.50 mmol, 1 equiv) and acid I-56 (181.26 mg, 0.75 mmol, 1.5 equiv) and TCFH (705.34 mg, 2.52 mmol, 5 equiv) and NMI (330.24 mg, 4.02 mmol, 8 equiv) and MeCN (5 mL) at room temperature. The resulting mixture was stirred for 2 h at room temperature under nitrogen atmosphere. The resulting mixture was concentrated under reduced pressure. The residue was purified by prep-TLC with CH₂Cl₂/methanol (27:1, v/v) to afford racemic amide C-129 (440 mg, 168.08%) (crude) as a brown oil. LCMS: m/z (ESI), [M+H]⁺=521.45.

Step 2: Into a 50 mL round-bottom flask were added racemic amide C-129 (430 mg, 0.83 mmol, 1 equiv) and TFA (1 mL) and DCM (3 mL) at room temperature. The resulting mixture was stirred for 2 h at room temperature under air atmosphere. The resulting mixture was concentrated under reduced pressure. The residue was dissolved in DMF (2 mL). The mixture was basified to pH 9 with NH₃ aq. The resulting mixture was stirred for 30 min at 0° C. under air atmosphere. The crude product (150 mg) was purified by prep-HPLC with the following conditions (Column: XBridge Shield RP18 OBD 30*150 mm, 5 um; Mobile Phase A: water (10 mmol/L NH₄HCO₃+0.1% NH₃ aq), Mobile Phase B: ACN; Flow rate: 60 mL/min; Gradient: 28% B to 50% B in 8 min; wavelength: 220 nm; t_{R}=7.75 min) to afford Example 129, racemic (26.9 mg, 7.70%) as a white solid. LCMS: m/z (ESI), [M+H]⁺=307.04. ¹H NMR (CD₃OD, 400 MHZ) δ 1.12 (2H, dt), 1.33 (3H, m), 1.77 (4H, d), 2.02 (2H, m), 2.31 (3H, s), 2.56 (4H, s), 2.83 (4H, m), 3.09 (1H, d), 7.09 (1H, t), 7.20 (1H, dd), 7.48 (1H, s), 7.65 (1H, dd), 7.93 (2H, s).

### Example 131 (Enantiomer 1) and Example 132 (Enantiomer 2)

### Preparation of N-(3-(1H-pyrazol-4-yl)-1H-indol-7-yl)-2-amino-2-(pyridin-3-yl) acetamide

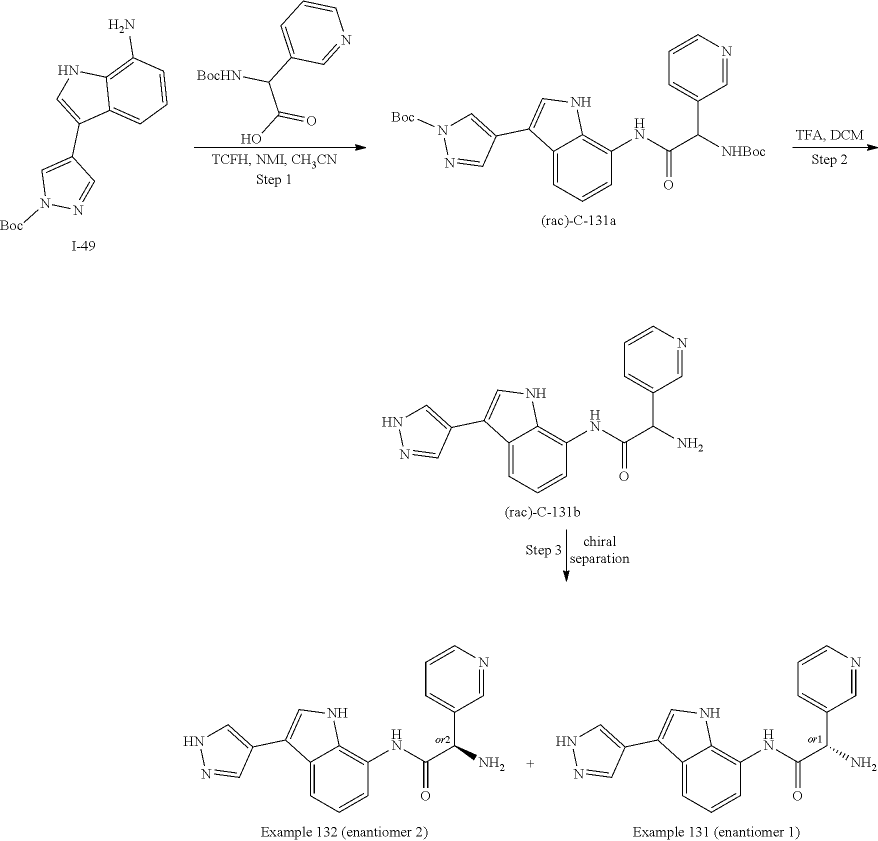

Step 1: Into a 40 mL vial were added [(tert-butoxycarbonyl)amino](pyridin-3-yl) acetic acid (250 mg, 0.99 mmol, 1 equiv) and amine I-49 (295.66 mg, 0.99 mmol, 1 equiv) and TCFH (333.67 mg, 1.19 mmol, 1.2 equiv) and NMI (325.47 mg, 3.96 mmol, 4 equiv) and MeCN (5 mL) at room temperature. The resulting mixture was stirred for 1 h at room temperature under nitrogen atmosphere. The resulting mixture was concentrated under reduced pressure. The residue was purified by prep-TLC with CH₂Cl₂/methanol (18:1, v/v) to afford racemic amide C131a (200 mg, 37.89%) as a Brown yellow solid. LCMS: m/z (ESI), [M+H]⁺=533.35.

Step 2: Into a 100 mL round-bottom flask were added racemic amide C131a (300 mg, 0.56 mmol, 1 equiv) and DCM (3 mL) and TFA (0.86 mL) at room temperature. The resulting mixture was stirred for 1 h at room temperature under air atmosphere. The resulting mixture was concentrated under reduced pressure. The residue was dissolved in DMF (2 mL). The mixture was basified to pH 9 with NH₃ (aq). The resulting mixture was stirred for 30 min at 0° C. under air atmosphere. The crude product (250 mg) was purified by prep-HPLC with the following conditions (Column: XBridge Shield RP18 OBD 30*150 mm, 5 um; Mobile Phase A: water (10 mmol/L NH₄HCO₃), Mobile Phase B: ACN; Flow rate: 60 mL/min; Gradient: 5% B to 30% B in 8 min; wavelength: 220 nm; t_{R}=7.93 min) to afford racemic amine C-131b (150 mg, 80.21%) as a white solid. LCMS: m/z (ESI), [M+H]⁺=333.3.

Step 3: The racemic amine C-131b (120 mg) was purified by prep-chiral HPLC with the following conditions (Column: CHIRALPAK IG, 2*25 cm, 5 um; Mobile Phase A: Hex (0.2% DEA), Mobile Phase B: EtOH:DCM (1:1, v/v); Flow rate: 20 mL/min; Gradient: 50% B to 50% B in 22 min; wavelength: 220/254 nm; t_{R}=12.23 min; t_{R}=14.06 min; Sample Solvent: EtOH:DCM (1:1, v/v); Injection Volume: 0.6 mL) to afford Example 132, enantiomer 2 (38 mg, 31.66%) as a white solid. LCMS: m/z (ESI), [M+H]⁺=333.20. ¹H NMR (CD₃OD, 400 MHZ) δ 5.11 (1H, q), 7.09 (1H, t), 7.17 (1H, td), 7.48 (1H, s), 7.56 (1H, ddd), 7.67 (1H, dd), 7.92 (2H, s), 8.13 (1H, dt), 8.62 (1H, dq), 8.83 (1H, d), SFC: t_{R}=2.398, ce=97.40%, and Example 131, enantiomer 1 (18.5 mg, 15.41%) as a white solid. LCMS: m/z (ESI), [M+H]⁺=333.15. ¹H NMR (CD₃OD, 400 MHZ) δ 5.11 (1H, q), 7.09 (1H, t), 7.17 (1H, td), 7.48 (1H, s), 7.56 (1H, ddd), 7.67 (1H, dd), 7.92 (2H, s), 8.13 (1H, dt), 8.62 (1H, dq), 8.83 (1H, d). SFC: t_{R}=2.682, ee=95.58%.

### Example 133 (Racemic)

### Preparation of N-(3-(1H-pyrazol-4-yl)-1H-indol-7-yl)-1,2,3,4-tetrahydroisoquinoline-4-carboxamide

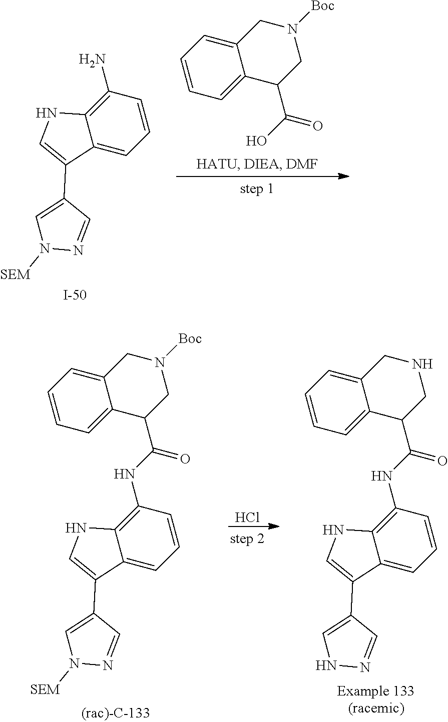

Step 1: A mixture of amine I-50 (100 mg, 0.30 mmol, 1 equiv), 2-(tert-butoxycarbonyl)-1,2,3,4-tetrahydroisoquinoline-4-carboxylic acid (1 equiv), HATU (231.50 mg, 0.61 mmol, 2 equiv) and DIEA (118.04 mg, 0.91 mmol, 3 equiv) in DMF (3 mL) was stirred for 2 h at room temperature under nitrogen atmosphere. The residue was purified by reverse flash chromatography with the following conditions: column, C18 silica; mobile phase, MeCN in water (0.1% NH₃·H₂O), 10% to 50% gradient in 10 min; detector, UV 254 nm to afford racemic amide C-133 (120 mg, 67.06%) as a yellow solid. LCMS: m/z (ESI), [M+H]⁺=588.25.

Step 2: To a solution of racemic amide C-133 (102 mg, 0.17 mmol, 1 equiv) was added HCl (gas) in 1,4-dioxane (10 mL) at 25° C. The mixture was stirred for 3 h at 40° C.° C. The resulting mixture was concentrated under reduced pressure. The crude product (80 mg) was purified by prep-HPLC with the following conditions (Column: XBridge Prep OBD C18 30*150 mm, 5 um; Mobile Phase A: water (10 mmol/L NH₄HCO₃), Mobile Phase B: ACN; Flow rate: 60 mL/min; Gradient: 12% B to 37% B in 8 min; wavelength: 254 nm; t_{R}=7.33 min) to afford Example 133, racemic (6.7 mg, 10.59%) as a white solid. LCMS: m/z (ESI), [M+H]⁺=358.10. ¹H NMR (DMSO-d₆, 400 MHz) δ 3.22 (1H, dd), 3.38 (2H, dd), 3.83 (1H, t), 3.90 (1H, d), 3.99 (1H, d), 7.00 (1H, t), 7.11 (1H, dd), 7.18 (2H, dt), 7.34 (1H, m), 7.42 (1H, d), 7.59 (2H, m), 7.94 (2H, s), 10.39 (1H, s), 10.69 (1H, m), 12.81 (1H, s).

### Example 134 (Racemic)

### Preparation of N-(2-amino-1-phenylethyl)-3-(1H-pyrazol-4-yl)-1H-indole-7-carboxamide

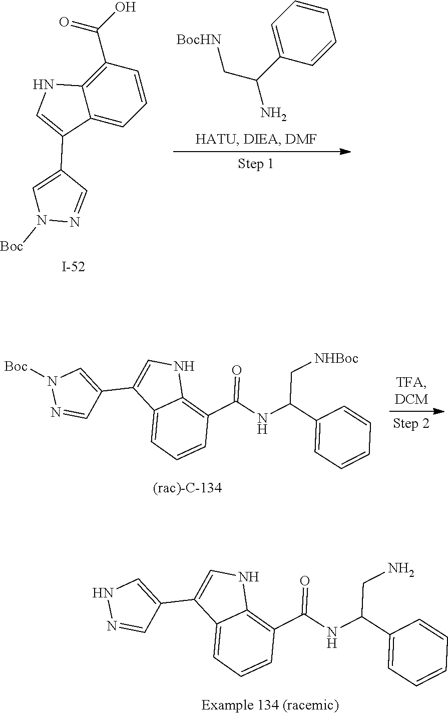

Step 1: Into a 40 mL vial were added acid I-52 (100 mg, 0.44 mmol, 1 equiv), tert-butyl N-(2-amino-2-phenylethyl) carbamate (155.97 mg, 0.66 mmol, 1.5 equiv), HATU (250.01 mg, 0.62 mmol, 1.5 equiv), TEA (150.26 mg, 1.32 mmol, 3 equiv) and DMF (20 mL) at room temperature. The resulting mixture was stirred for 8 h at room temperature, The resulting mixture was diluted with EtOAc (30 mL). The resulting mixture was washed with 3×30 ml of water. The organic layer was dried over anhydrous Na₂SO₄. After filtration, the filtrate was concentrated under reduced pressure. The residue was purified by prep-TLC with CH₂Cl₂/MeOH (15:1, v/v) to afford racemic amide C-134 (120 mg, 61.22%) as a light yellow solid. LCMS: m/z (ESI), [M+H]⁺=446.40.

Step 5: Into a 50 mL round-bottom flask were added racemic amide C-134 (100 mg, 0.23 mmol, 1 equiv), TFA (1 mL) and DCM (3 mL) at room temperature. The resulting mixture was stirred for 1 h at room temperature. The mixture was concentrated under reduced pressure and basified to pH 9 with saturated NaHCO₃ (aq.). The resulting mixture was extracted with CH₂Cl₂/MeOH (10:1, v/v) (3×30 mL), dried over anhydrous Na₂SO₄. After filtration, the filtrate was concentrated under reduced pressure. The crude product (80 mg) was purified by prep-HPLC with the following conditions (Column: XBridge Shield RP18 OBD Column, 30*150 mm, 5 um; Mobile Phase A: water (10 mmol/L NH₄HCO₃+0.1% NH₃·H₂O), Mobile Phase B: ACN; Flow rate: 60 mL/min; Gradient: 17% B to 37% B in 8 min, 37% B; wavelength: 220 nm; t_{R}=7.85 min) to afford Example 134, racemic (21.6 mg, 27.87%) as a white solid. LCMS: m/z (ESI), [M+H]⁺=346.15. ¹H NMR (400 MHZ, CD₃OD) δ 3.12 (2H, m), 5.28 (1H, dd), 7.23 (1H, t), 7.30 (1H, m), 7.39 (2H, m), 7.48 (3H, m), 7.82 (1H, dd), 7.97 (2H, m), 8.00 (1H, d).

### Example 135

### Preparation of(S)—N-(2-amino-1-phenylethyl)-3-(1H-pyrazol-4-yl)-1H-indole-7-carboxamide

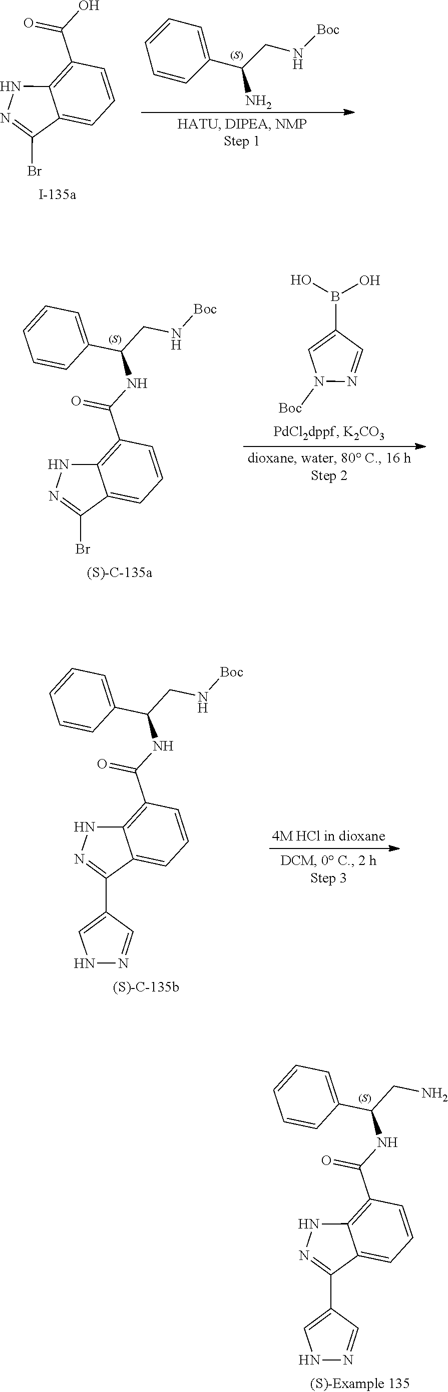

Step 1: To a mixture of acid I-135a (48.20 mg, 199.95 μmol), tert-butyl(S)-(2-amino-2-phenylethyl) carbamate (45 mg, 190.43 μmol) and N,N-diethylethanamine (57.81 mg, 571.29 μmol, 79.63 μL) in NMP (2.00 mL) was stirred and cooled at 0° C. by ice/water bath. Then added HATU (86.89 mg, 228.51 μmol) in one portion at this temperature. The resulting mixture was stirred and warmed slowly to room temperature for 2 h. The reaction solution was purified by C18 flash chromatography column, elution gradient from 0 to 50% acetonitrile in water. Pure fractions were evaporated to dryness to afford tert-butyl N-[(2S)-2-[(3-bromo-1H-indazole-7-carbonyl)amino]-2-phenyl-ethyl]carbamate C-135a (82 mg, 94% yield) as yellow solid. LCMS: t_{R}=1.91 min in 3 min chromatography (3 min-5-95% MeCN in water (0.02% TFA), Waters Acquity UPLC BEH C18 1.7 μm, 2.1*50 mm, 40° C.), MS (ESI) m/z=459.1 [M+H]⁺.

Step 2: tert-butyl N-[(2S)-2-[(3-bromo-1H-indazole-7-carbonyl)amino]-2-phenyl-ethyl]carbamate C-135a (82 mg, 178.52 μmol), (1-(tert-Butoxycarbonyl)-1H-pyrazol-4-yl) boronic acid (45.42 mg, 214.22 μmol), Potassium carbonate (74.02 mg, 535.56 μmol, 32.32 μL) and PdCl₂dppf (13.06 mg, 17.85 μmol) in dioxane (1.50 mL) and water (0.30 mL) were stirred and heated at 80° C. for 16 h under nitrogen. The reaction solution was evaporated under reduced pressure. The residue was purified by silica flash chromatography column, elution gradient from 10 to 50% under nitrogen tert-butyl N-[(2S)-2-phenyl-2-[[3-(1H-pyrazol-4-yl)-1H-indazole-7-carbonyl]amino]ethyl]carbamate C-135b (75 mg, 94% yield) as brown solid. LCMS: t_{R}=1.61 min in 3 min chromatography (3 min-5-95% MeCN in water (0.02% TFA), Waters Acquity UPLC BEH C18 1.7 μm, 2.1*50 mm, 40° C.), MS (ESI) m/z=447.4 [M+H]⁺.

Step 3: To a solution of tert-butyl N-[(2S)-2-phenyl-2-[[3-(1H-pyrazol-4-yl)-1H-indazole-7-carbonyl]amino]ethyl]carbamate C-135b (75 mg, 167.97 μmol) in DCM (3.00 mL) was stirred and cooled at 0° C. by ice/water bath. Then added 4 M HCl (145.76 mg, 4.00 mmol) in dioxane (1.00 mL) dropwise into the reaction. The resulting mixture was stirred at this temperature for 2 h. The reaction mixture was concentrated under reduced pressure. The residue was purified by C18 flash chromatography column, elution gradient from 0% to 60% MeCN in water (6 mmol/L NH₄HCO₃). Pure fractions were lyophilized to dryness to afford N-[(1S)-2-amino-1-phenyl-ethyl]-3-(1H-pyrazol-4-yl)-1H-indazole-7-carboxamide Example 135 (37 mg, 64% yield) as white solid. LCMS: t_{R}=1.16 min in 3 min chromatography (3 min-5-95% MeCN in water (6 mmol/L NH₄HCO₃), Waters Acquity UPLC BEH C18 1.7 μm, 2.1*50 mm, 40° C.), MS (ESI) m/z=347.2 [M+H]⁺. ¹H NMR: (400 MHz, DMSO-d₆) δ 13.12 (s, 1H), 8.99 (s, 1H), 8.25 (d, J=8.0 Hz, 2H), 8.11 (d, J=7.2 Hz, 1H), 7.41 (d, J=7.3 Hz, 2H), 7.33 (t, J=7.6 Hz, 2H), 7.25 (dt, J=11.8, 7.4 Hz, 2H), 5.05 (qd, J=6.9, 3.1 Hz, 1H), 2.95 (ddd, J=18.2, 13.0, 7.0 Hz, 2H).

### Example 136 (Enantiomer 1) and Example 139 (Enantiomer 2)

### Preparation of N-(2-amino-1-(3-methoxyphenyl)ethyl)-3-(1H-pyrazol-4-yl)-1H-indole-7-carboxamide

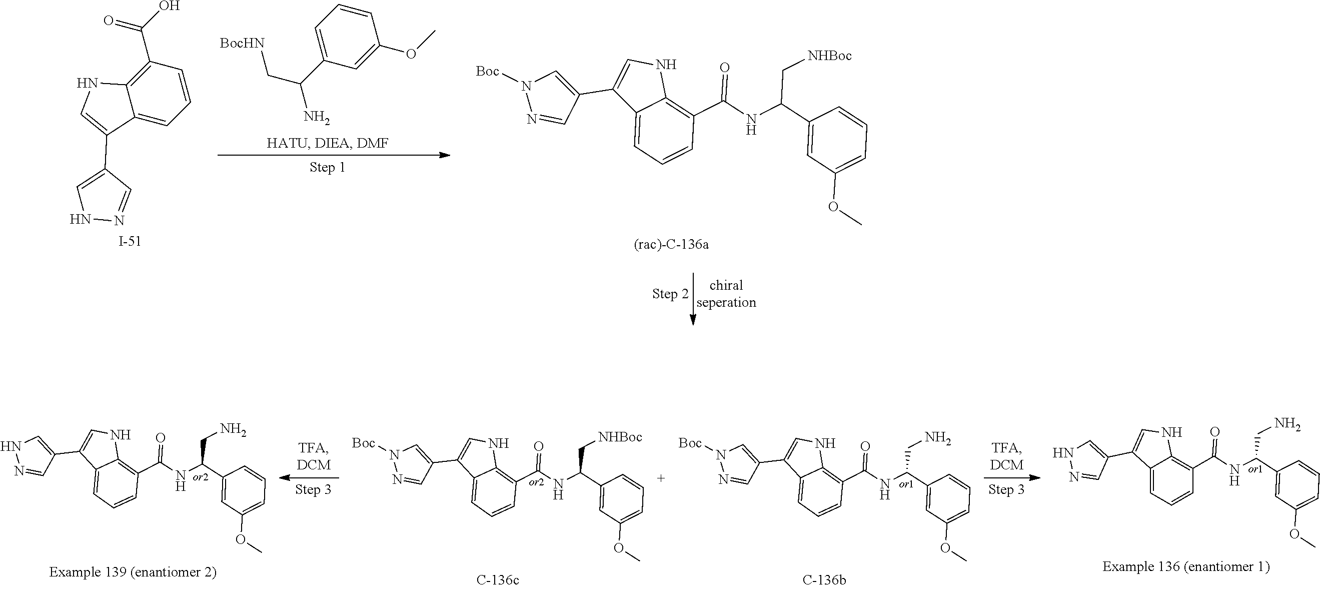

Step 1: To a stirred solution of acid I-51 (100 mg, 0.44 mmol, 1 equiv) and tert-butyl N-[2-amino-2-(3-methoxyphenyl)ethyl]carbamate (117.22 mg, 0.44 mmol, 1 equiv) in DMF (5 mL) were added HATU (251.01 mg, 0.66 mmol, 1.5 equiv) and DIEA (170.64 mg, 1.32 mmol, 3 equiv) in portions at room temperature under nitrogen atmosphere. The resulting mixture was stirred for 2 h at room temperature under nitrogen atmosphere. The resulting mixture was concentrated under vacuum. The crude product was purified by reverse flash chromatography with the following conditions: column, C18 silica; mobile phase, MeCN in water, 0% to 100% gradient in 25 min; detector, UV 254 nm. The resulting mixture was concentrated under vacuum. This resulted in racemic amide C-136a (70 mg, 33.45%) as a white solid. LCMS: m/z (ESI), [M+H]⁺=476.10.

Step 2: The racemic amide C-136a was purified by chiral HPLC with the following conditions (Column: CHIRALPAK IG, 2*25 cm, 5 um; Mobile Phase A: Hex (0.2% DEA), Mobile Phase B: EtOH:DCM (1:1, v/v); Flow rate: 20 mL/min; Gradient: 80% B to 80% B in 12 min; wavelength: 220/254 nm; t_{R}−1=6.18 min; t_{R}−2=9.16 min; Sample Solvent: EtOH:DCM (1:1, v/v); Injection Volume: 0.55 mL) to afford enantiomer C-136c (25 mg) as a white solid. SCF: t_{R}=1.464 min, ee=100% and enantiomer C-136b (25 mg) as a white solid. SCF: t_{R}=2.09 min, ee=100%.

Step 3: A solution of enantiomer C-136b (25 mg, 0.053 mmol, 1 equiv) and TFA (1 mL) in DCM (2 mL) was stirred for 2 h at room temperature under nitrogen atmosphere. The resulting mixture was concentrated under vacuum. The crude product was purified by prep-HPLC with the following conditions (Column: XBridge Shield RP18 OBD 30*150 mm, 5 um; Mobile Phase A: water (10 mmol/L NH₄HCO₃+0.1% NH₃ aq), Mobile Phase B: ACN; Flow rate: 60 mL/min; Gradient: 15% B to 40% B in 8 min; wavelength: 220 nm; t_{R}=7.82 min) to afford Example 136, enantiomer 1 (4.9 mg, 24.83%) as a white solid. LCMS: m/z (ESI), [M+H]⁺=376.10. ¹H NMR (400 MHZ, DMSO-d₆) δ 2.94 (2H, qd), 3.74 (3H, s), 5.11-4.97 (1H, m), 6.85-6.76 (1H, m), 6.99 (2H, dd), 7.16 (1H, d), 7.25 (1H, t), 7.51 (1H, s), 8.14-7.79 (4H, m), 8.77 (1H, d), 11.21 (1H, d), 12.81 (1H, s).

A solution of enantiomer C-136c (25 mg, 0.053 mmol, 1 equiv) and TFA (1 mL) in DCM (2 mL) was stirred for 2 h at room temperature under nitrogen atmosphere. The resulting mixture was concentrated under vacuum. The crude product was purified by prep-HPLC with the following conditions (Column: XBridge Prep OBD 30*150 mm, 5 um; Mobile Phase A: water (10 mmol/L NH₄HCO₃), Mobile Phase B: ACN; Flow rate: 60 mL/min; Gradient: 14% B to 39% B in 8 min; wavelength: 220 nm; t_{R}=6.82 min) to afford Example 139, enantiomer 2 (4.9 mg, 19.08%) as a white solid. LCMS: m/z (ESI), [M+H]⁺=376.10. ¹H NMR (400 MHZ, DMSO-d₆) δ 3.15 (2H, dd), 3.75 (3H, s), 5.24 (1H, s), 6.84-6.34 (2H, m), 6.86 (1H, dd), 7.09-6.98 (2H, m), 7.19 (1H, t), 7.29 (1H, t), 7.52 (1H, s), 7.89 (2H, d), 8.03 (2H, d), 8.90 (1H, d), 11.14 (1H, s), 12.84 (1H, s).

### Example 137 (Enantiomer 1) and Example 138 (Enantiomer 2)

### Preparation of N-(1-(3-methoxyphenyl)-2-(4-methylpiperazin-1-yl)ethyl)-3-(1H-pyrazol-4-yl)-1H-indole-7-carboxamide

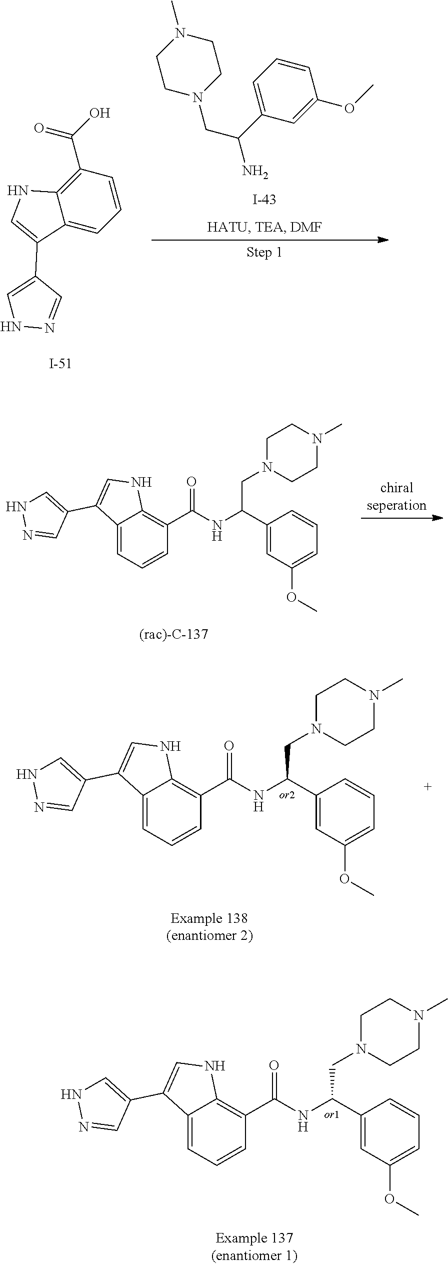

Step 1: Into a 8 mL vial were added amine I-43 (109.74 mg, 0.44 mmol, 1 equiv), acid I-51 (100 mg, 0.44 mmol, 1 equiv), triethylamine (133.60 mg, 1.32 mmol, 3 equiv) in DMF (2 mL) and HATU (251.01 mg, 0.66 mmol, 1.5 equiv) was added to the above mixture at room temperature. The resulting mixture was stirred for 1 h at room temperature under nitrogen atmosphere. The resulting mixture was diluted with EtOAc (20 mL), and washed with saturated NaHCO₃ (aq) (20 mL), water (20 mL) and saturated brine (2×20 mL), and the organic layer was dried over anhydrous Na₂SO₄, filtered and evaporated to afford a crude solid. The crude product (150 mg) was purified by reverse flash chromatography with the following conditions: column, C18 silica; mobile phase, methanol in water (10 mmol/L, NH₄HCO₃), 0% to 100% gradient in 60 min; detector, UV 254 nm. to afford racemic amide C-137 (80 mg, 39.64%) as a yellow solid. LCMS: m/z (ESI), [M+H]⁺=459.10.

Step 2: The racemic amide C-137 (80 mg) was purified by prep-chiral HPLC with the following conditions (Column: DZ-CHIRALPAK IC-3, 4.6*50 mm, 3.0 um; Mobile Phase A: Hex (0.2% DEA): (EtOH:DCM=1:1, v/v)=60:40 (v/v); Flow rate: 1 mL/min; Injection Volume: 5 ul mL) to afford Example 137, enantiomer 1 (19.5 mg, 23.52%) as a white solid. LCMS: m/z (ESI), [M+H]⁺=459.20. ¹H NMR (CD₃OD, 400 MHZ) & 2.32 (3H, s), 2.45-2.86 (9H, m), 2.98 (1H, d), 3.81 (3H, s), 5.40 (1H, dd), 6.84 (1H, d), 7.04 (2H, d), 7.26 (2H, dt), 7.54 (1H, s), 7.74 (1H, d), 7.89 (1H, s), 7.99 (2H, d). Chiral HPLC: t_{R}=1.655 min, ee=100% and Example 138, enantiomer 2 (16.5 mg, 20.37%) as a white solid. LCMS: m/z (ESI), [M+H]⁺=459.20. ¹H NMR (CD₃OD, 400 MHZ) δ 2.32 (3H, s), 2.45-2.86 (9H, m), 2.98 (1H, d), 3.81 (3H, s), 5.40 (1H, dd), 6.84 (1H, d), 7.04 (2H, d), 7.26 (2H, dt), 7.54 (1H, s), 7.74 (1H, d), 7.89 (1H, s), 7.99 (2H, d). Chiral HPLC: t_{R}=2.002 min, ee=97.88%.

### Example 140 (Racemic)

### Preparation of N-(3-amino-1-phenylpropyl)-3-(1H-pyrazol-4-yl)-1H-indole-7-carboxamide

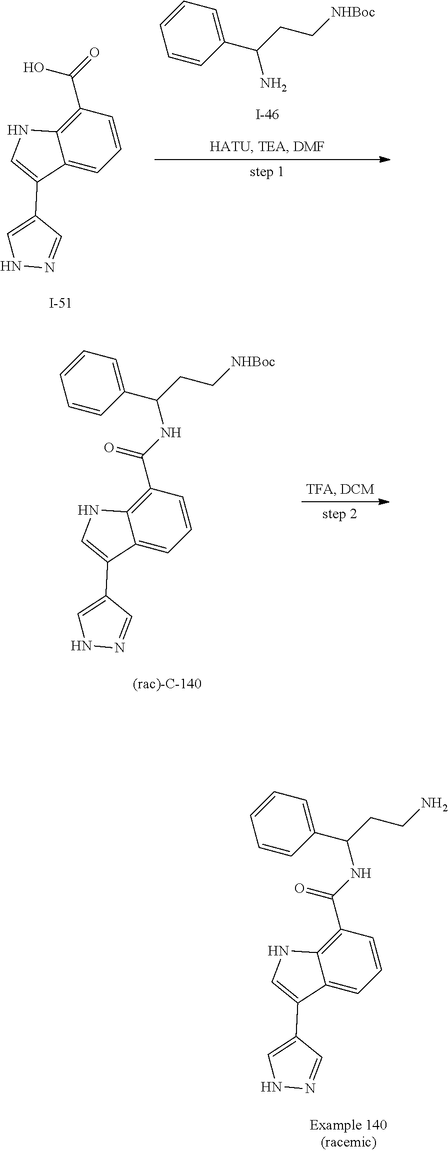

Step 1: Into a 20 mL vial were added amine I-46 (250 mg, 1.00 mmol, 1 equiv), HATU (379.72 mg, 1.00 mmol, 1 equiv), triethylamine (303.16 mg, 3.00 mmol, 3 equiv), and acid I-51 (226.91 mg, 1.00 mmol, 1 equiv) in DMF (2.50) at room temperature. The resulting mixture was stirred for 1 h at room temperature under nitrogen atmosphere. The residue was diluted with EtOAc (20 mL), washed with saturated NaHCO₃ (aq.) (1×20 mL) and saturated brine (3×20 mL), The organic layer was concentrated under reduced pressure. The residue was purified by prep-TLC with CH₂Cl₂/methanol (10:1, v/v) to afford racemic amide C-140 (250 mg, 54.48%) as a yellow solid. LCMS: m/z (ESI), [M+H]⁺=460.35. ¹H NMR (DMSO-d₆, 400 MHz) δ 1.38 (9H, s), 2.01 (2H, dddd), 3.01 (2H, dq), 5.03-5.18 (1H, m), 6.92 (1H, t), 7.16 (1H, t), 7.20-7.27 (1H, m), 7.34 (2H, t), 7.42-7.47 (2H, m), 7.51 (1H, d), 7.84 (1H, d), 8.00 (1H, d).

Step 2: Into a 50 mL round-bottom flask were added racemic amide C-140 (250 mg, 0.54 mmol, 1 equiv) and TFA (1 mL) in DCM (3 ml) at room temperature. The resulting mixture was stirred for 1.5 h at room temperature. The resulting mixture was concentrated under reduced pressure. The mixture was basified to pH 9 with NH₃ aq. The crude product was purified by prep-HPLC with the following conditions (Column: XBridge Shield RP18 OBD 30*150 mm, 5 um; Mobile Phase A: water (10 mmol/L NH₄HCO₃), Mobile Phase B: ACN; Flow rate: 60 mL/min; Gradient: 9% B to 39% B in 8 min; wavelength: 220 nm; t_{R}=7.82 min) to afford Example 140, racemic (90.7 mg, 46.39%) as a white solid. LCMS: m/z (ESI), [M+H]⁺=360.20. ¹H NMR (CD₃OD, 400 MHZ) δ 2.12-2.27 (2H, m), 2.77-3.03 (2H, m), 5.37 (1H, dd), 7.21 (1H, t), 7.25-7.33 (1H, m), 7.36-7.43 (2H, m), 7.45-7.54 (3H, m), 7.75 (1H, dd), 7.94 (2H, s), 7.99 (1H, dd).

### Example 141 (Enantiomer 1) and Example 142 (Enantiomer 2)

### Preparation of N-((3-methoxyphenyl) (1-methylpiperidin-4-yl)methyl)-3-(1H-pyrazol-4-yl)-1H-indole-7-carboxamide

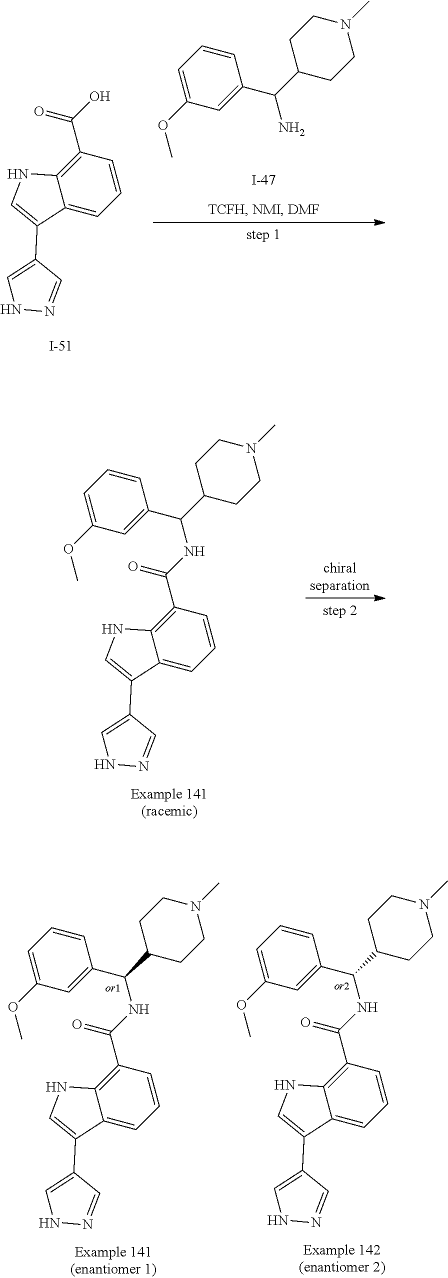

Step 1: To a stirred mixture of acid I-51 (148.35 mg, 0.65 mmol, 1.02 equiv) in CH₃CN (4.0 mL) was added amine I-47 (150 mg, 0.64 mmol, 1 equiv), NMI (157.67 mg, 1.92 mmol, 3 equiv) and TCFH (359.19 mg, 1.28 mmol, 2 equiv) in portions at 25° C. under air atmosphere. The resulting mixture was stirred for 2.0 h at 25° C. under air atmosphere. After reaction, the resulting mixture was diluted with water (20 mL). The resulting mixture was extracted with ethyl acetate (3×30 mL). The combined organic layers were washed with water (3×10 mL), dried over anhydrous Na₂SO₄. After filtration, the filtrate was concentrated under reduced pressure. The residue was purified by prep-TLC with CH₂Cl₂/methanol (20:1, v/v) to afford racemic amide C-141 (200 mg, 70.44%) as a white solid. LCMS: m/z (ESI), [M+H]⁺=444.20.

Step 2: The racemic amide C-141 (200 mg) was purified by prep-chiral HPLC with the following conditions (Column: DZ-CHIRALPAK IG-3, 4.6*50 mm, 3.0 um; Mobile Phase A: Hex (0.2% DEA)/(EtOH:DCM, 1:1, v/v)=50:50 (v/v); Flow rate: 1 mL/min; Injection Volume: 5 ul mL) to afford Example 141, enantiomer 1 (42 mg, 21.66%). LCMS: m/z (ESI), [M+H]⁺=444.25. ¹H NMR (CD₃OD, 400 MHZ) δ 1.35 (2H, d), 1.51 (1H, dt), 1.97 (2H, m), 2.09 (2H, d), 2.31 (3H, d), 2.88 (1H, d), 3.00 (1H, d), 3.82 (3H, s), 4.98 (1H, m), 6.84 (1H, m), 7.04 (2H, m), 7.20 (1H, t), 7.28 (1H, t), 7.50 (1H, s), 7.73 (1H, dd), 7.96 (3H, m). SCF: t_{R}=2.644 min, ee=100% and Example 142, enantiomer 2 (42.8 mg, 22.08%) as a white solid. LCMS: m/z (ESI), [M+H]⁺=444.20. ¹H NMR (CD₃OD, 400 MHZ) δ 1.35 (2H, d), 1.51 (1H, dt), 1.97 (2H, m), 2.09 (2H, d), 2.31 (3H, d), 2.88 (1H, d), 3.00 (1H, d), 3.82 (3H, s), 4.97 (1H, m) 6.84 (1H, m), 7.04 (2H, m), 7.20 (1H, t), 7.28 (1H, t), 7.50 (1H, s), 7.73 (1H, dd), 7.96 (3H, m). SFC: t_{R}=3.725 min, ee=100%.

### Example 143 (Racemic)

### Preparation of N-[cyclohexyl (1-methylpiperidin-4-yl)methyl]-3-(1H-pyrazol-4-yl)-1H-indole-7-carboxamide

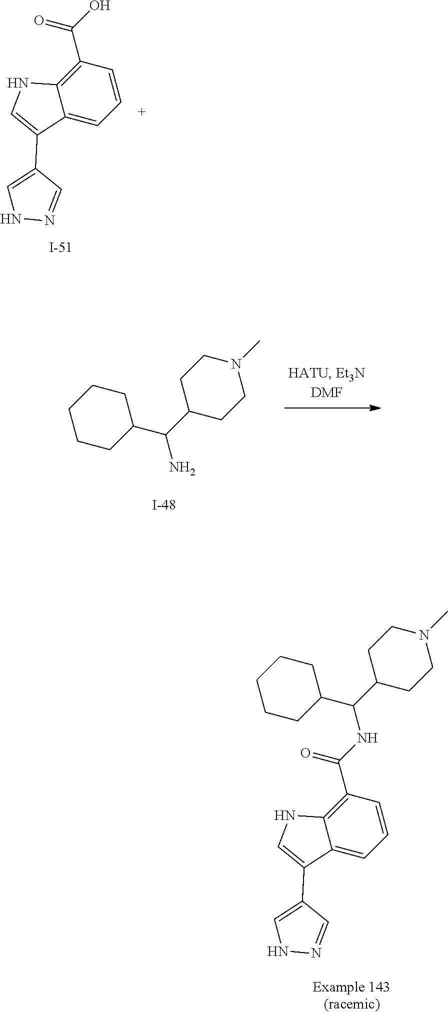

Into a 20 mL vial were added amine I-48 (111.10 mg, 0.53 mmol, 1.50 equiv) HATU (133.87 mg, 0.35 mmol, 1 equiv), triethylamine (106.88 mg, 1.06 mmol, 3 equiv) and acid I-51 (80 mg, 0.35 mmol, 1 equiv) in DMF (1.5 mL) at room temperature. The resulting mixture was stirred for 1.5 h at room temperature under nitrogen atmosphere. The residue was diluted with EA (50 mL), and washed with saturated NaHCO₃ (aq.) (1×50 mL), and saturated brine (2×50 mL). The organic layer was concentrated under reduced pressure. The residue was purified by prep-TLC with CH₂Cl₂/methanol (5:1, v/v). The crude product was purified by prep-HPLC with the following conditions (Column: XBridge Shield RP18 OBD 30*150 mm, 5 um; Mobile Phase A: water (10 mmol/L NH₄HCO₃+0.1% NH₃ aq), Mobile Phase B: ACN; Flow rate: 60 mL/min; Gradient: 25% B to 48% B in 8 min; wavelength: 220 nm; t_{R}=8.10 min) to afford Example 143, racemic (28.3 mg, 19.16%) as a white solid. LCMS: m/z (ESI), [M+H]⁺=420.30. ¹H NMR (CD₃OD, 400 MHZ) & 1.02-1.41 (5H, m), 1.51 (2H, p), 1.63-1.94 (9H, m), 2.01-2.19 (2H, m), 2.30 (3H, s), 2.98 (2H, d), 3.98 (1H, t), 7.20 (1H, t), 7.53 (1H, s), 7.84-8.03 (3H, m).

### Example 144 (Enantiomer 1) and Example 145 (Enantiomer 2)

### Preparation of N-(2-amino-1-(4-methoxyphenyl)ethyl)-3-(1H-pyrazol-4-yl)-1H-indole-7-carboxamide

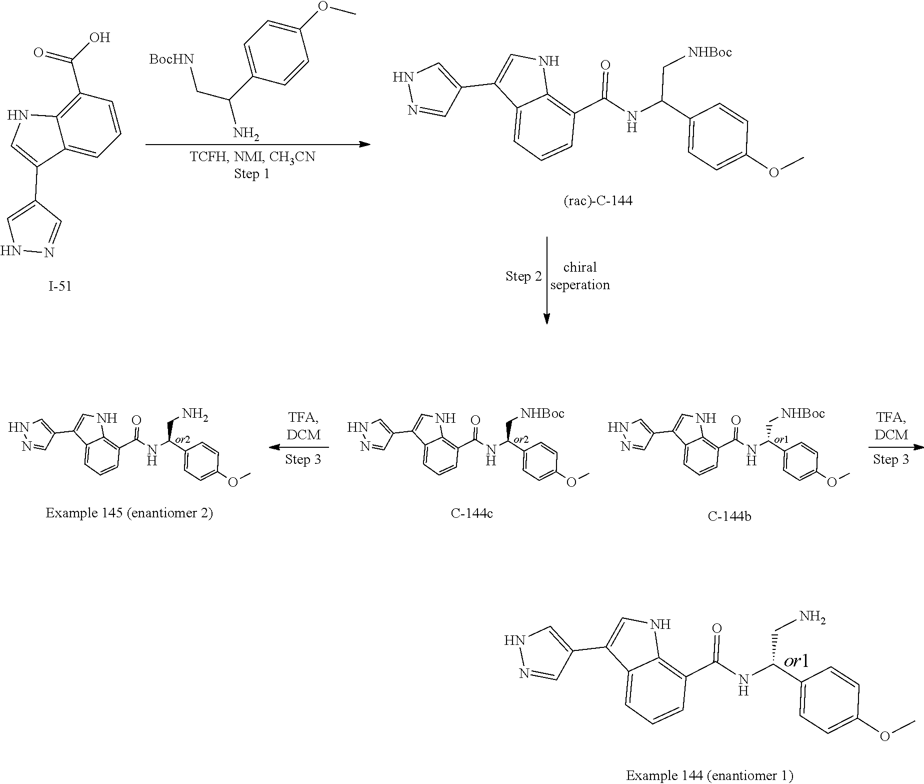

Step 1: A mixture of acid I-51 (300 mg, 1.32 mmol, 1 equiv) and tert-butyl N-[2-amino-2-(4-methoxyphenyl)ethyl]carbamate (351.65 mg, 1.32 mmol, 1 equiv), TCFH (555.67 mg, 1.98 mmol, 1.5 equiv), NMI (379.41 mg, 4.62 mmol, 3.5 equiv) in MeCN (5 mL) was stirred for overnight at room temperature under nitrogen atmosphere. The resulting mixture was diluted with water (80 mL). The resulting mixture was extracted with CH₂Cl₂ (3×100 mL). The combined organic layers were dried over anhydrous Na₂SO₄. After filtration, the filtrate was concentrated under reduced pressure. The residue was purified by prep-TLC with CH₂Cl₂/methanol (18:1, v/v) to afford racemic C-144a (370 mg, 58.93%) as a brown solid. LCMS: m/z (ESI), [M+H]⁺=476.25.

Step 2: racemic C-144a (370 mg) (crude, purity=82%) was purified with prep-chiral HPLC with the following conditions (Column: CHIRALPAK IG, 2*25 cm, 5 um; Mobile Phase A: Hex (0.2% DEA), Mobile Phase B: EtOH:DCM (1:1, v/v); Flow rate: 20 mL/min; Gradient: 80% B, 15 min; wavelength: 220/254 nm; t_{R}−1=5.17 min; t_{R}−2=11.31 min; Sample Solvent: EtOH:DCM (1:1, v/v); Injection Volume: 1.4 mL) to afford enantiomer C-144b (100 mg, 31.02%) and enantiomer C-144c (110 mg, 35.46%) as a white solid.

Step 3: Into a 50 mL round-bottom flask were added enantiomer C-144b (60 mg, 0.13 mmol, 1 equiv) in DCM (3 mL) and TFA (1 mL) at room temperature. The resulting mixture was stirred for 1 h at room temperature under nitrogen atmosphere. The resulting mixture was concentrated under reduced pressure. The residue was dissolved in DCM (3 mL). The resulting mixture was concentrated under reduced pressure. The mixture was basified to pH 9 with NH₃ (aq). The crude product (50 mg) was purified by prep-HPLC with the following conditions (Column: XBridge Shield RP18 OBD 30*150 mm, 5 um; Mobile Phase A: water (10 mmol/L NH₄HCO₃+0.1% NH₃·H₂O), Mobile Phase B: ACN; Flow rate: 60 mL/min; Gradient: 18% B to 38% B in 8 min; wavelength: 220 nm; t_{R}=7.60 min) to afford Example 144, enantiomer 1 (24.8 mg, 52.36%) as a white solid. LCMS: m/z (ESI), [M+H]⁺=376.05. ¹H NMR (DMSO-d₆, 400 MHz) δ 2.88 (1H, dd), 2.92-3.03 (1H, m), 3.73 (3H, s), 4.86-5.26 (1H, m), 6.82-6.95 (2H, m), 7.16 (1H, td), 7.26-7.41 (2H, m), 7.51 (1H, d), 7.84 (1H, dd), 8.00 (2H, d), 8.53-9.03 (1H, m). SFC: t_{R}=2.027 min, ee=98.92%.

Into a 50 mL round-bottom flask were added enantiomer C-144c (60 mg, 0.13 mmol, 1 equiv) and TFA (1 mL, 13.46 mmol), DCM (3 mL, 47.19 mmol) at room temperature. The resulting mixture was stirred for 1 h at room temperature. The resulting mixture was concentrated under reduced pressure. The resulting mixture was diluted with DMF (5 mL). The residue was basified to pH 9 with saturated NH₃ (aq). The crude product was purified by prep-HPLC with the following conditions (Column: XBridge Prep OBD 30*150 mm, 5 um; Mobile Phase A: water (10 mmol/L NH₄HCO₃), Mobile Phase B: ACN; Flow rate: 60 mL/min; Gradient: 13% B to 38% B in 8 min; wavelength: 220 nm) to afford Example 145, enantiomer 2 (46.9 mg, 98.55%) as a white solid. LCMS: m/z (ESI), [M+H]⁺=376.2. ¹H NMR (400 MHZ, DMSO-d₆) δ 2.87 (dd, 1H), 2.96 (dd, 1H), 3.73 (s, 3H), 4.99 (d, 1H), 6.90 (m, 2H), 7.15 (td, 1H), 7.34 (dd, 2H), 7.51 (s, 1H), 7.85 (d, 1H), 8.00 (d, 3H), 8.74 (d, 1H), 11.13 (s, 1H). SCF: t_{R}=1.697 min, ee=100%.

### Example 146 (Racemic)

### Preparation of N-(2-amino-1-(4-(hydroxymethyl)phenyl)ethyl)-3-(1H-pyrazol-4-yl)-1H-indole-7-carboxamide

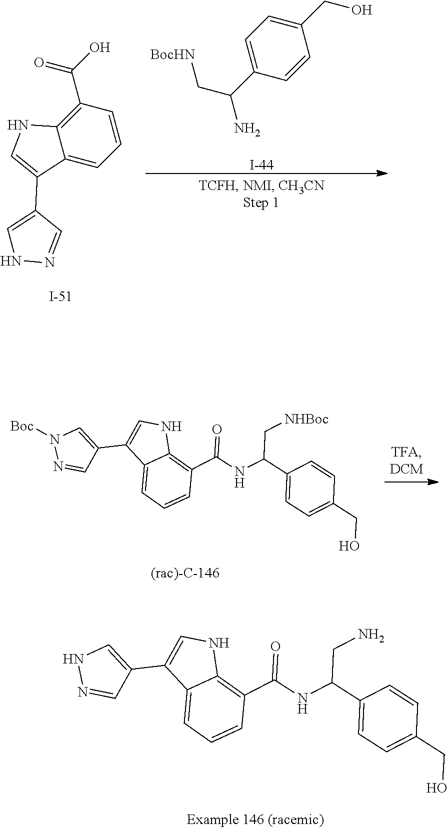

Step 1: To a stirred mixture of amine I-44 (120 mg, 0.45 mmol, 1 equiv) and acid I-51 (102.38 mg, 0.45 mmol, 1 equiv) in MeCN (3 mL) were added TCFH (189.62 mg, 0.68 mmol, 1.5 equiv) and NMI (147.97 mg, 1.80 mmol, 4 equiv) in portions at room temperature under nitrogen atmosphere for 1 h. The resulting mixture was concentrated under vacuum. The residue was purified by reverse flash chromatography with the following conditions: column, C18 silica; mobile phase, MeCN in water, 0% to 100% gradient in 60 min; detector, UV 254 nm. The resulting mixture was concentrated under vacuum. The crude product was purified by prep-HPLC with the following conditions to afford racemic amide C-146 (30 mg, 14.00%) as a white solid. ¹H NMR (400 MHZ, DMSO-d₆) δ 1.36 (9H, s), 3.30-3.48 (1H, m), 4.47 (2H, d), 5.13-5.21 (1H, m), 7.05-7.23 (2H, m), 7.27 (2H, d), 7.30-7.45 (3H, m), 7.52 (1H, dd), 7.82 (1H, d), 8.00 (3H, s), 8.01 (1H, s), 8.76 (1H, d), 11.12 (1H, d).

Step 2: To a stirred mixture of racemic amide C-146 (20 mg, 0.042 mmol, 1 equiv) and TFA (4.80 mg, 0.042 mmol, 1 equiv) in DCM (1 mL) at room temperature under nitrogen atmosphere for 1.5 h. The mixture was basified to pH 8 with saturated Na₂CO₃ (aq.). The precipitated solids were collected by filtration and washed with DCM (20 mL). The resulting mixture was concentrated under reduced pressure. The crude product was purified by prep-HPLC with the following conditions (Column: XBridge Prep OBD C18 30*150 mm, 5 um; Mobile Phase A: water (10 mmol/L NH₄HCO₃), Mobile Phase B: ACN; Flow rate: 60 mL/min; Gradient: 5% B to 25% B in 7 min; wavelength: 220 nm; t_{R}=7.63 min) to afford Example 146, racemic (8.3 mg, 52.57%) as a white solid. LCMS: m/z (ESI), [M+H]⁺=376.15. ¹H NMR (400 MHZ, DMSO-d₆) δ 2.89-3.06 (1H, m), 4.46 (2H, d), 5.06 (1H, s), 5.12 (1H, t), 7.17 (1H, t), 7.27 (2H, d), 7.36 (2H, d), 7.51 (1H, s), 7.88 (1H, d), 8.01 (1H, d), 8.80 (1H, d).

### Example 147 (Racemic)

### Preparation of N-(1-amino-3-phenylpropan-2-yl)-3-(1H-pyrazol-4-yl)-1H-indole-7-carboxamide

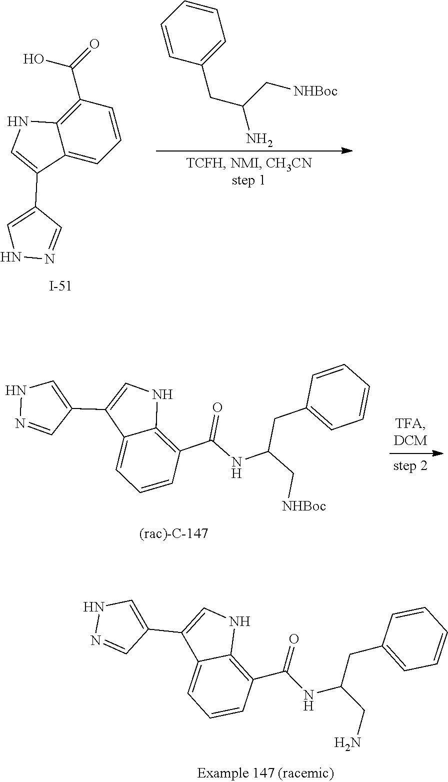

Step 1: Into a 20 mL vial were added acid I-51 (150 mg, 0.66 mmol, 1 equiv) and tert-butyl N-(2-amino-3-phenylpropyl) carbamate (247.89 mg, 0.99 mmol, 1.5 equiv), TCFH (222.27 mg, 0.79 mmol, 1.2 equiv), NMI (216.81 mg, 2.64 mmol, 4 equiv) in MeCN (5.00 mL) at room temperature. The resulting mixture was stirred for 2 h at room temperature under nitrogen atmosphere. The resulting mixture was concentrated under reduced pressure. The residue was dissolved in DCM (5 mL). The residue was purified by prep-TLC with CH₂Cl₂/methanol (15:1, v/v) to afford racemic amide C147 (302 mg, 89.59%) as a light yellow oil. LCMS: m/z (ESI), [M+H]⁺=460.10.

Step 2: Into a 50 mL round-bottom flask were added racemic amide C-147 (300 mg, 0.65 mmol, 1 equiv) in TFA (1 mL) and DCM (3 mL) at room temperature. The resulting mixture was stirred for 1 h at room temperature under nitrogen atmosphere. The resulting mixture was concentrated under reduced pressure. The residue was dissolved in methanol (3 mL). The mixture was basified to pH 9 with NH₃ aq. The crude product (150 mg) was purified by prep-HPLC with the following conditions (Column: XBridge Shield RP18 OBD 30*150 mm, 5 um; Mobile Phase A: water (10 mmol/L NH₄HCO₃+0.1% NH₃ aq), Mobile Phase B: ACN; Flow rate: 60 mL/min; Gradient: 18% B to 38% B in 8 min; wavelength: 220 nm; t_{R}=7.77 min) to afford Example 147, racemic (105.3 mg, 44.88%) as a white solid. LCMS: m/z (ESI), [M+H]⁺=360.15. ¹H NMR (DMSO-d₆, 400 MHZ) δ 2.74 (1H, s), 2.81-3.02 (2H, m), 3.19 (1H, d), 4.05-4.32 (1H, m), 7.01-7.21 (2H, m), 7.21-7.39 (4H, m), 7.52 (1H, s), 7.68 (1H, dd), 8.12 (3H, dd).

### Example 148

### Preparation of N-[(1S)-2-amino-1-phenyl-ethyl]-3-(1H-pyrazol-4-yl)-1H-pyrrolo[3,2-c]pyridine-7-carboxamide

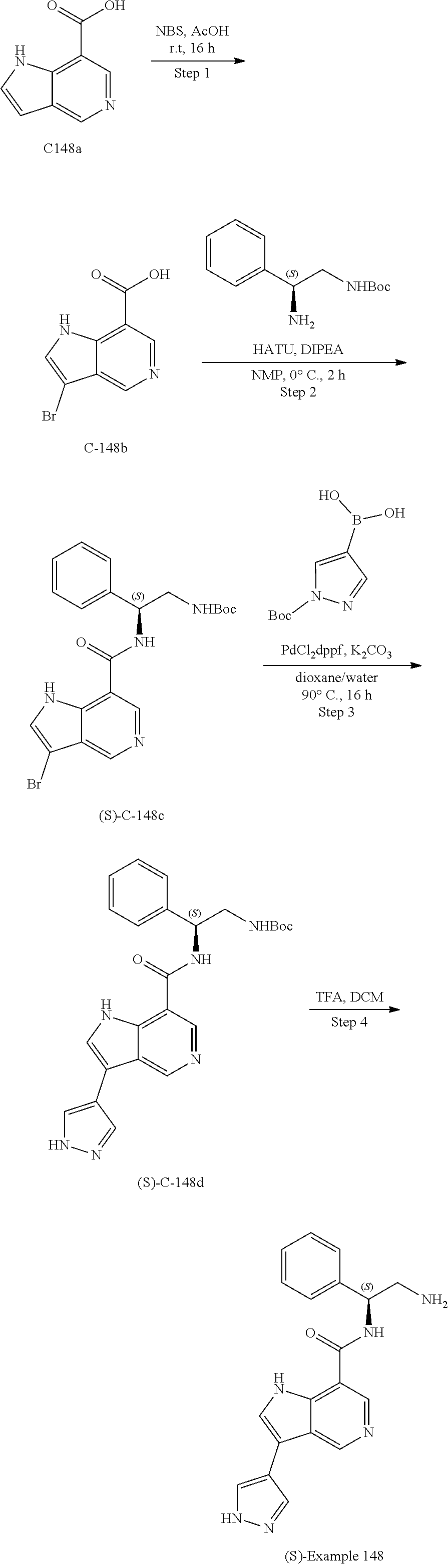

Step 1: To a solution of acid C₁₄₈a (250 mg, 1.54 mmol) in AcOH (10 mL) was added N-bromosuccinimide (329.31 mg, 1.85 mmol, 156.96 μL), the reaction mixture was stirred at room temperature for 16 h. The reaction solution was evaporated under reduced pressure. The residue was purified by C18 flash chromatography column, elution gradient from 0 to 20% MeCN in water. Pure fractions were lyophilized to dryness to afford 3-bromo-1H-pyrrolo[3,2-c]pyridine-7-carboxylic acid C₁₄₈b (200 mg, 54% yield) as white solid. LCMS: t_{R}=0.77 min in 3 min chromatography (5-95% MeCN in water (0.02% TFA), waters Acquity UPLC BEH C18 1.7 μm, 2.1*50 mm, 40° C.), MS (ESI) m/z=240.8 [M+H]⁺.

Step 2: To a stirred mixture of 3-bromo-1H-pyrrolo[3,2-c]pyridine-7-carboxylic acid C-148b (50 mg, 207.43 μmol), tert-butyl N-[(2S)-2-amino-2-phenyl-ethyl]carbamate (49.02 mg, 207.43 μmol) and N,N-diethylethanamine (104.95 mg, 1.04 mmol, 144.56 μL) in DMF (1.44 mL) was cooled and stirred at 0° C. Then HATU (118.31 mg, 311.15 μmol) was added in one portion at this temperature. The resulting mixture was stirred at 0° C. for 2 h. The solution was purified by C18 flash chloramatography column, elution gradient from 0 to 60% MeCN in water. Pure fractions were lyophilized to dryness to afford tert-butyl N-[(2S)-2-[(3-bromo-1H-pyrrolo[3,2-c]pyridine-7-carbonyl)amino]-2-phenyl-ethyl]carbamate C-148c (60 mg, 63% yield) as yellow solid. LCMS: t_{R}=1.45 min in 3 min chromatography (5-95% MeCN in water (0.02% TFA), waters Acquity UPLC BEH C18 1.7 μm, 2.1*50 mm, 40° C.), MS (ESI) m/z=459.1 [M+H]⁺.

Step 3: tert-butyl N-[(2S)-2-[(3-bromo-1H-pyrrolo[3,2-c]pyridine-7-carbonyl)amino]-2-phenyl-ethyl]carbamate C-148c (60 mg, 130.62 μmol), (1-(tert-butoxycarbonyl)-1H-pyrazol-4-yl) boronic acid (41.54 mg, 195.94 μmol), K₂CO₃ (54.16 mg, 391.87 μmol) and Pd(dppf)Cl₂ (10.67 mg, 13.06 μmol) in dioxane (0.5 mL) and water (3 mL) were stirred under nitrogen at 90° C. for 16 h. The solvent was removed under reduced pressure. The residue was purified by silica flash chromatography column, elution gradient from 0 to 10% methanol in DCM. Pure fractions were evaporated to dryness to afford tert-butyl N-[(2S)-2-phenyl-2-[[3-(1H-pyrazol-4-yl)-1H-pyrrolo[3,2-c]pyridine-7-carbonyl]amino]ethyl]carbamate C-148d (40 mg, 69% yield) as brown solid. LCMS: t_{R}=1.39 min in 3 min chromatography (5-95% MeCN in water (0.02% TFA), waters Acquity UPLC BEH C18 1.7 μm, 2.1*50 mm, 40° C.), MS (ESI) m/z=447.3 [M+H]⁺.

Step 4: To a solution of tert-butyl N-[(2S)-2-phenyl-2-[[3-(1H-pyrazol-4-yl)-1H-pyrrolo[3,2-c]pyridine-7-carbonyl]amino]ethyl]carbamate C-148d (40 mg, 89.59 μmol) in DCM (1.00 mL) was cooled at 0° C. in ice/water bath, and stirred. Then added trifluoroacetic acid (510.74 mg, 4.48 mmol, 345.10 μL) dropwise into the reaction. The resulting mixture was stirred for 2 h. The reaction mixture was concentrated under reduced pressure. The residue was purified by C18-flash chromatography, elution gradient from 0% to 50% MeCN in water (6 mmol/L NH₄HCO₃). Pure fractions were lyophilized to dryness to afford N-[(1S)-2-amino-1-phenyl-ethyl]-3-(1H-pyrazol-4-yl)-1H-pyrrolo[3,2-c]pyridine-7-carboxamide Example 148 (25 mg, 81% yield) as white solid. LCMS: t_{R}=0.87 min in 3 min chromatography (5-95% MeCN in water (0.02% TFA), waters Acquity UPLC BEH C18 1.7 μm, 2.1*50 mm, 40° C.), MS (ESI) m/z=347.5 [M+H]⁺. ¹H NMR: (400 MHZ, DMSO-d₆) δ 12.90 (s, 1H), 9.22 (s, 1H), 8.99 (d, J=7.2 Hz, 1H), 8.91 (s, 1H), 8.09 (s, 2H), 7.59 (s, 1H), 7.41 (d, J=7.5 Hz, 2H), 7.33 (t, J=7.5 Hz, 2H), 7.23 (t, J=7.2 Hz, 1H), 5.03 (dd, J=11.8, 6.1 Hz, 1H), 3.05-2.84 (m, 2H).

### Example 149 (Racemic)

### Preparation of N-[2-amino-1-(pyridin-3-yl)ethyl]-3-(1H-pyrazol-4-yl)-1H-indole-7-carboxamide

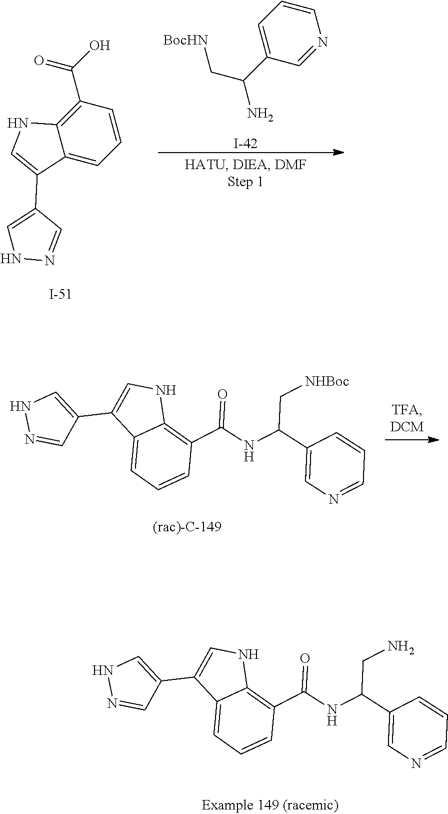

Step 1: To a stirred mixture of acid I-51 (130 mg, 0.572 mmol, 1.00 equiv) and DIEA (221.84 mg, 1.716 mmol, 3.00 equiv), amine I-42 (149.34 mg, 0.629 mmol, 1.1 equiv) in DMF (2.0 mL) was added and HATU (337.19 mg, 0.887 mmol, 1.55 equiv) in portions at room temperature under nitrogen atmosphere. The resulting mixture was stirred for 1 hour at room temperature. After reaction, 10 mL water was added to the mixture. The resulting mixture was extracted with ethyl acetate (3×30 mL). The combined organic layers were washed with water (3×10 mL), dried over anhydrous Na₂SO₄. After filtration, the filtrate was concentrated under reduced pressure. The residue was purified by prep-TLC with CH₂Cl₂/methanol (12:1, v/v) to afford racemic amide C-149 (130 mg, 50.89%) as a yellow oil. LCMS: m/z (ESI), [M+H]⁺=447.15.

Step 2. To a stirred solution of racemic amide C-149 (125 mg, 0.280 mmol, 1 equiv) in DCM (4.0 mL) was added TFA (1.0 mL) dropwise at room temperature under nitrogen atmosphere. After reaction, the resulting mixture was concentrated under vacuum and dissolved in methanol (2.0 mL). The mixture was basified to pH 9 with NH₃ aq (1.0 mL) at 0° C. The resulting mixture was submitted to prep-HPLC with the following conditions (Column: XBridge Shield RP18 OBD 30*150 mm, 5 um; Mobile Phase A: water 10 mmol/L NH₄HCO₃), Mobile Phase B: ACN; Flow rate: 60 mL/min; Gradient: 5% B to 28% B in 8 min; wavelength: 220/254 nm; t_{R}=7.97 min) to afford Example 149, racemic (26.7 mg, 26.70%) as a white solid. LCMS: m/z (ESI), [M+H]⁺=347.20. ¹H NMR (CD₃OD, 400 MHz) δ 3.16 (2H, m), 5.32 (1H, dd), 7.23 (1H, t), 7.49 (2H, m), 7.83 (1H, m), 7.98 (4H, m), 8.48 (1H, dd), 8.68 (1H, d).

### Example 151 (Racemic)

### Preparation of (rac)-N-{2-amino-1-[3-(dimethylphosphoryl)phenyl]ethyl}-3-(1H-pyrazol-4-yl)-1H-indole-7-carboxamide

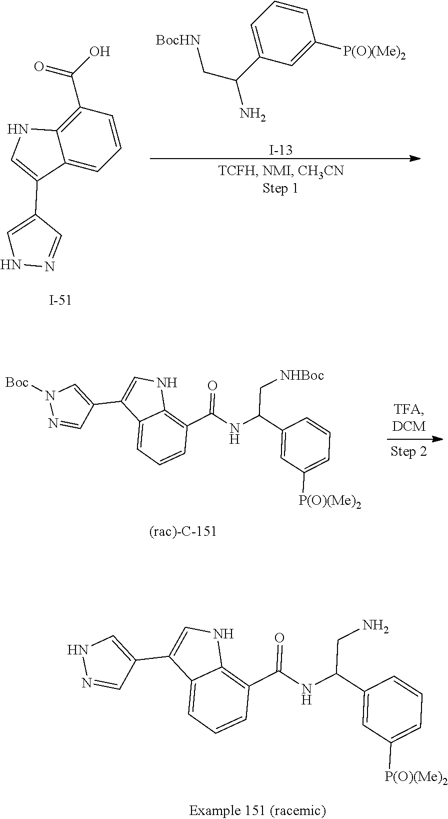

Step 1: To a stirred mixture of amine I-13 (50 mg, 0.16 mmol, 1 equiv) and acid I-51 (36.37 mg, 0.16 mmol, 1 equiv) in MeCN (3 mL) were added TCFH (53.90 mg, 0.19 mmol, 1.2 equiv) and NMI (52.57 mg, 0.64 mmol, 4 equiv) in portions at room temperature under nitrogen atmosphere. The resulting mixture was stirred for 1 h at room temperature under nitrogen atmosphere. The resulting mixture was concentrated under vacuum. The residue was purified by reverse flash chromatography with the following conditions: column, C18 silica; mobile phase, MeCN in water (0.1% FA), 0% to 100% gradient in 10 min; detector, UV 254 nm. This resulted in racemic amide C-151 (30 mg, 35.93%) as a white solid. ¹H NMR (400 MHZ, DMSO-d₆) δ 1.31 (9H, s), 1.64 (6H, d), 3.30 (1H, s), 3.44 (1H, dd), 5.26 (1H, q), 7.16 (2H, dt), 7.44-7.53 (2H, m), 7.62 (2H, dt), 7.81-7.90 (2H, m), 8.01 (1H, d), 8.09 (1H, s), 8.89 (1H, d), 11.14 (1H, d), 12.81 (1H, s).

Step 2: To a stirred mixture of racemic amide C-151 (20 mg, 0.038 mmol, 1 equiv) in CH₂Cl₂ (2 mL) was added TFA (4.37 mg, 0.038 mmol, 1.0 equiv) at room temperature under nitrogen atmosphere. The resulting mixture was stirred for 1 h at room temperature. The resulting mixture was concentrated under vacuum. The crude product was purified by prep-HPLC with the following conditions (Column: XBridge Prep OBD 30*150 mm, 5 um; Mobile Phase A: water (10 mmol/L NH₄HCO₃), Mobile Phase B: ACN; Flow rate: 60 mL/min; Gradient: 5% B to 25% B in 7 min; wavelength: 220 nm; t_{R}=7.63 min) to afford Example 151, racemic (5.2 mg, 32.18%) as a white solid. LCMS: m/z (ESI), [M+H]⁺=422.25. ¹H NMR (400 MHZ, DMSO-d₆) δ 1.62 (3H, s), 1.65 (3H, s), 2.89-3.06 (2H, m), 5.08 (1H, d), 7.18 (1H, t), 7.37-7.54 (2H, m), 7.54-7.68 (2H, m), 7.74-7.94 (3H, m), 8.01 (2H, d), 8.88 (1H, d), 11.17 (1H, s), 12.82 (1H, s).

### Example 152 (Racemic)

### Preparation of 3-amino-2-(3-cyanophenyl)-N-[7-(1H-pyrazol-4-yl)-5H-pyrrolo[3,2-d]pyrimidin-4-yl]propanamide

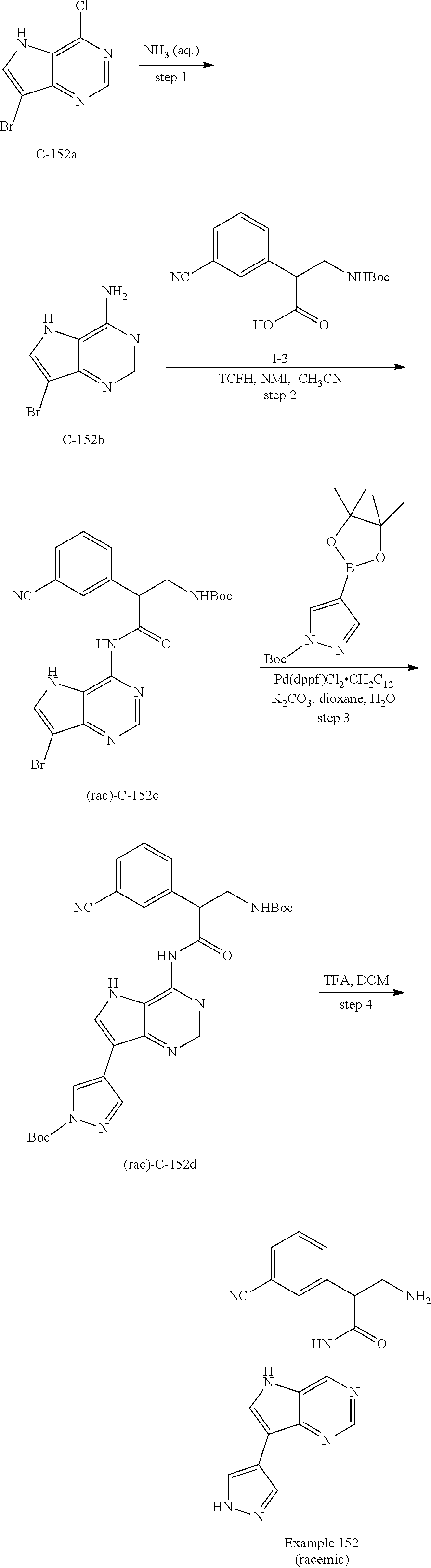

Step 1: Into a 40 mL vial were added 7-bromo-4-chloro-5H-pyrrolo[3,2-d]pyrimidine C-152a (1 g, 4.30 mmol, 1 equiv) and NH₃ aq (20 mL) at room temperature. The resulting mixture was stirred for 72 h at 100° C. under air atmosphere. The product was precipitated by the addition of water. The precipitated solids were collected by filtration and washed with water (3×10 mL). The resulting solid was dried by lyophilization. This resulted in 7-bromo-5H-pyrrolo[3,2-d]pyrimidin-4-amine C-152b (0.84 g, 91.66%) as a brown solid. LCMS: m/z (ESI), [M+H]⁺=212.95. ¹H NMR (CD₃OD, 400 MHZ) δ 7.60 (1H, s), 8.19 (1H, s).

Step 2: Into a 40 mL vial were added amine C-152b (232.37 mg, 1.09 mmol, 1 equiv) and acid I-3 (380.00 mg, 1.31 mmol, 1.2 equiv) and TCFH (1.84 g, 6.545 mmol, 6 equiv) and NMI (716.46 mg, 8.73 mmol, 8 equiv) and MeCN (8 mL) at room temperature. The resulting mixture was stirred for 1 h at room temperature under nitrogen atmosphere. The resulting mixture was filtered and the filtered cake was washed with CH₂Cl₂ (3×10 mL). The filtrate was concentrated under reduced pressure. The residue was purified by prep-TLC with CH₂Cl₂/methanol (30:1, v/v) to afford a crude solid. The residue was purified by reverse flash chromatography with the following conditions: column, C18 silica; mobile phase, methanol in water (0.1% FA), 50% to 70% gradient in 20 min; detector, UV 254 nm. The resulting mixture was concentrated under reduced pressure. This resulted in racemic amide C-152c (290 mg, 54.78%) as a brown yellow solid. LCMS: m/z (ESI), [M+H]⁺=487.10.

Step 3: Into a 20 mL vial were added racemic amide C-152c (250 mg, 0.52 mmol, 1 equiv) and tert-butyl 4-(4,4,5,5-tetramethyl-1,3,2-dioxaborolan-2-yl) pyrazole-1-carboxylate (393.96 mg, 1.34 mmol, 2.6 equiv) and Pd(dppf)Cl₂·CH₂Cl₂ (125.88 mg, 0.15 mmol, 0.3 equiv) and K₂CO₃ (213.57 mg, 1.55 mmol, 3 equiv) and 1,4-dioxane (8 mL) and H₂O (2 mL) at room temperature. The resulting mixture was stirred for 2 h at 80° C. under nitrogen atmosphere. The resulting mixture was diluted with water (100 mL). The aqueous layer was extracted with ethyl acetate (3×100 mL). The resulting mixture was concentrated under reduced pressure. The residue was purified by prep-TLC with CH₂Cl₂/methanol (20:1, v/v) to afford racemic pyrazole C-152d (100 mg, 33.90%) as a yellow solid. LCMS: m/z (ESI), [M+H]⁺=573.15.

Step 4: Into a 8 mL vial were added racemic pyrazole C-152d (100 mg, 0.17 mmol, 1 equiv) and TFA (1 mL, 13.46 mmol, 77.93 equiv) and DCM (3 mL) at room temperature. The resulting mixture was stirred for 1 h at room temperature under air atmosphere. The resulting mixture was concentrated under reduced pressure. The residue was dissolved in DMF (1 mL). The mixture was basified to pH 9 with NH₃ aq. The resulting mixture was stirred for 30 min at 0° C. The crude product (65 mg) was purified by prep-HPLC with the following conditions (Column: XBridge Shield RP18 OBD 30*150 mm, 5 um; Mobile Phase A: water (10 mmol/L NH₄HCO₃), Mobile Phase B: ACN; Flow rate: 60 mL/min; Gradient: 9% B to 34% B in 8 min; wavelength: 220 nm; t_{R}=7.47 min) to afford Example 152, racemic (4 mg, 55.89%) as a white solid. LCMS: m/z (ESI), [M+H]⁺=373.15. ¹H NMR (CD₃OD, 400 MHz) δ 3.04 (1H, dd), 3.43 (1H, dd), 4.10 (1H, dd), 7.56 (1H, t), 7.68 (1H, dt), 7.79 (1H, dt), 7.87 (1H, d), 7.93 (1H, s), 8.18 (2H, m), 8.54 (1H, s).

### Example 153 (Enantiomer 1) and Example 154 (Enantiomer 2)

### Preparation of N-(7-(1H-pyrazol-4-yl)-5H-pyrrolo[3,2-d]pyrimidin-4-yl)-3-amino-2-(3-methoxyphenyl)propanamide

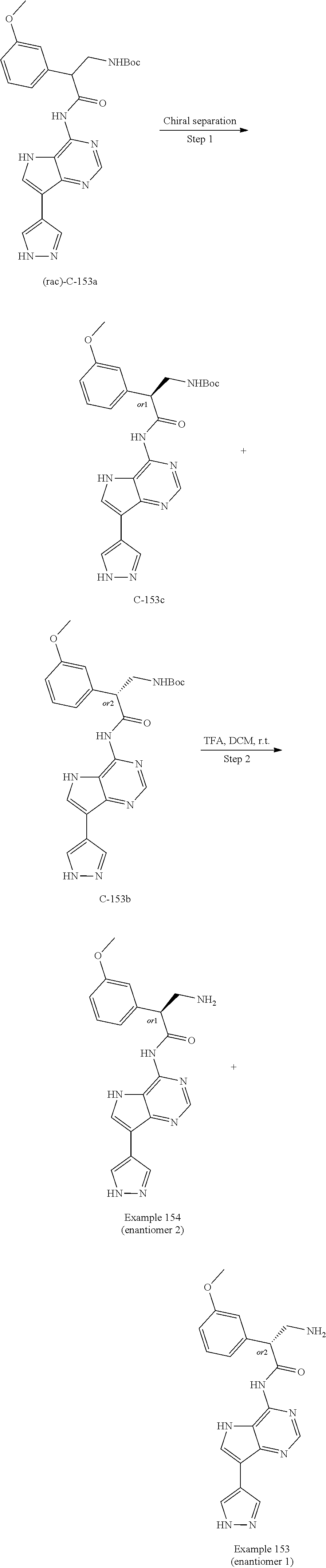 [SMILES: COc1cccc(C(CNC(=O)OC(C)(C)C)C(=O)Nc2ncnc3c(-c4cn[nH]c4)c[nH]c23)c1 ; COc1cccc([C@@H](CNC(=O)OC(C)(C)C)C(=O)Nc2ncnc3c(-c4cn[nH]c4)c[nH]c23)c1 ; COc1cccc([C@H](CNC(=O)OC(C)(C)C)C(=O)Nc2ncnc3c(-c4cn[nH]c4)c[nH]c23)c1 ; COc1cccc([C@@H](CN)C(=O)Nc2ncnc3c(-c4cn[nH]c4)c[nH]c23)c1 ; COc1cccc([C@H](CN)C(=O)Nc2ncnc3c(-c4cn[nH]c4)c[nH]c23)c1]

Step 1: The racemic amide C-153a (220 mg, 0.46 mmol, 1 equiv) was purified by prep-chiral HPLC with the following conditions (Column: CHIRAL ART Cellulose-SB, 4.6*100 mm, 3 um; Mobile Phase A: Hex (0.2% DEA): (EtOH:DCM=1:1, v/v)=70:30 (v/v); Flow rate: 1 mL/min; Gradient: 0% B to 0% B; Injection Volume: 5 ul mL) to afford enantiomer C-153c (64.5 mg, 29.32%) as a yellow solid, SFC: t_{R}=2.862 min, ee=100%, and enantiomer C-153b (74.3 mg, 33.77%) as a yellow solid, SFC: t_{R}=3.979 min, ee=97.83%.

Step 2: A solution of enantiomer C-153c (55.0 mg, 0.12 mmol, 1 equiv) and TFA (1.0 mL) in DCM (4.0 mL) was stirred for 30 min at room temperature under nitrogen atmosphere. After reaction. The resulting mixture was concentrated under vacuum and basified to pH 9 with NH₃ aq (0.5 mL) at 0° C. The resulting mixture was purified by prep-HPLC with the following conditions (Column: XBridge Prep OBD 30*150 mm, 5 um; Mobile Phase A: water (10 mmol/L NH₄HCO₃), Mobile Phase B: ACN; Flow rate: 60 mL/min; Gradient: 10% B to 33% B in 7 min; wavelength: 220 nm) to afford Example 154, enantiomer 2 (15.8 mg, 36.35%) as a yellow solid. LCMS m/z (ESI), [M+H]⁺=378.15. ¹H NMR (CD₃OD, 400 MHz) δ 3.05 (1H, dd), 3.45 (1H, dd), 3.82 (3H, s), 4.05 (1H, dd), 6.89 (1H, m), 7.06 (2H, dt), 7.31 (1H, t), 7.95 (1H, s), 8.15 (2H, s), 8.54 (1H, s). SFC: t_{R}=2.027 min, ee=51.30%.

A solution of enantiomer C-153b (55.0 mg, 0.12 mmol, 1 equiv) and TFA (1.0 mL) in DCM (4.0 mL) was stirred for 30 min at room temperature under nitrogen atmosphere. The resulting mixture was concentrated under vacuum and basified to pH 9 with NH₃ aq (0.5 mL). The resulting mixture was purified by prep-HPLC with the following conditions (Column: XBridge Prep OBD 30*150 mm, 5 um; Mobile Phase A: water (10 mmol/L NH₄HCO₃), Mobile Phase B: ACN; Flow rate: 60 mL/min; Gradient: 10% B to 33% B in 7 min; wavelength: 220 nm; t_{R}=6.55 min) to afford Example 153, enantiomer 1 (19.2 mg, 44.17%) as a yellow solid. LCMS: m/z (ESI), [M+H]⁺=378.15. ¹H NMR (CD₃OD, 400 MHz) δ 2.95 (1H, dd), 3.05 (1H, dd), 3.26 (1H, dd), 3.45 (1H, dd), 3.70 (2H, s), 3.81 (5H, d), 4.05 (1H, dd), 6.89 (3H, m), 7.05 (2H, m), 7.28 (2H, m), 7.95 (1H, s), 8.18 (2H, d), 8.55 (1H, s). SFC: t_{R}=2.143 min, ee=54.82%.

### Example 155

### Preparation of (2S)-3-amino-2-phenyl-N-[7-(1H-pyrazol-4-yl)-5H-pyrrolo[3,2-d]pyrimidin-4-yl]propanamide

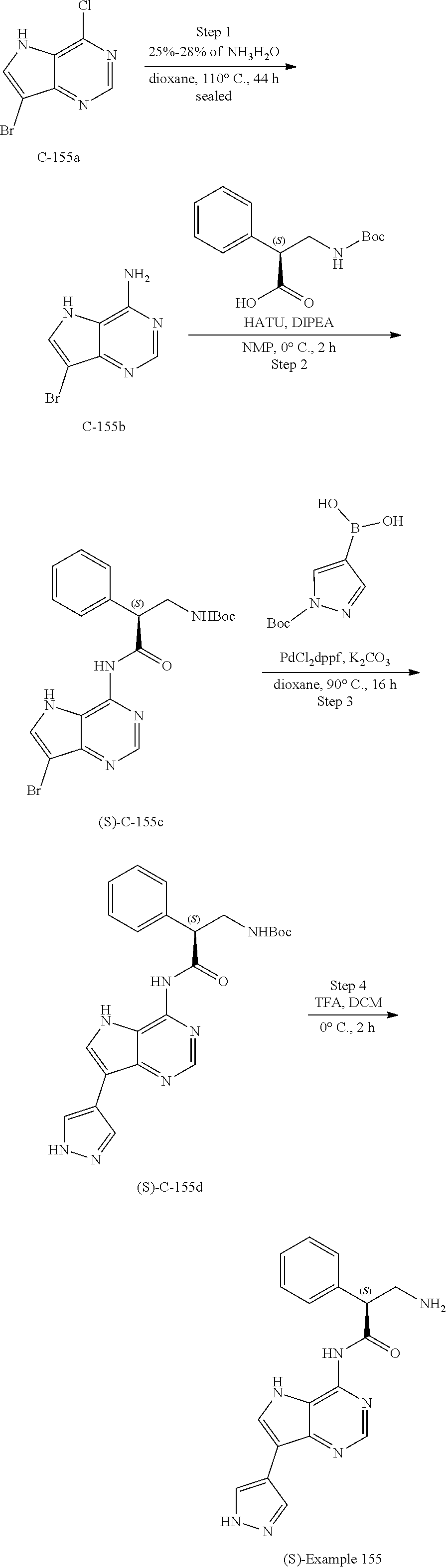

Step 1: A solution of 7-Bromo-4-chloro-5H-pyrrolo[3,2-d]pyrimidine C-155a (204 mg, 877.55 μmol) and 25%-28% of NH₃ aq (900.00 mg, 25.68 mmol, 1.00 mL) in dioxane (3 mL) was sealed and heated at 110° C. for 44 h. The reaction was evaporated under reduced pressure. The residue was purified by C18 flash chromatography column, elution gradient from 0 to 10% acetonitrile in water. Pure fractions were lyophilized to dryness to afford 7-bromo-5H-pyrrolo[3,2-d]pyrimidin-4-amine C-155b (120 mg, 64% yield) as brown solid. LCMS: t_{R}=0.75 min in 3 min chromatography (5-95% MeCN in water (0.02% TFA), waters Acquity UPLC BEH C18 1.7 μm, 2.1*50 mm, 40° C.), MS (ESI) m/z=212.9 [M+H]⁺.

Step 2: (2S)-3-(tert-butoxycarbonylamino)-2-phenyl-propanoic acid (224.16 mg, 844.93 μmol), 7-bromo-5H-pyrrolo[3,2-d]pyrimidin-4-amine C-155b (120 mg, 563.29 μmol), N-ethyl-N-isopropyl-propan-2-amine (218.40 mg, 1.69 mmol, 294.34 μL) in NMP (2.95 mL) The reaction mixture was stirred and cooled at 0° C. by ice/water bath. Then added HATU (278.43 mg, 732.28 μmol) into the reaction. The resulting mixture was stirred at this temperature for 2 h. The reaction solution was purified by C18 flash chromatography column, elution gradient from 0% to 80% MeCN in water. Pure fractions were lyophilized to dryness to afford tert-butyl N-[(2S)-3-[(7-bromo-5H-pyrrolo[3,2-d]pyrimidin-4-yl)amino]-3-oxo-2-phenyl-propyl]carbamate C-155c (45 mg, 17% yield) as brown solid. LCMS: t_{R}=1.65 min in 3 min chromatography (5-95% MeCN in water (0.02% TFA), waters Acquity UPLC BEH C18 1.7 μm, 2.1*50 mm, 40° C.), MS (ESI) m/z=460.1 [M+H]⁺.

Step 3: tert-butyl N-[(2S)-3-[(7-bromo-5H-pyrrolo[3,2-d]pyrimidin-4-yl)amino]-3-oxo-2-phenyl-propyl]carbamate C-155c (45 mg, 97.76 μmol), (1-(tert-Butoxycarbonyl)-1H-pyrazol-4-yl) boronic acid (31.09 mg, 146.64 μmol), K₂CO₃ (40.53 mg, 293.27 μmol) and Pd(dppf)Cl₂ (7.98 mg, 9.78 μmol) in dioxane (3.00 mL) and water (0.50 mL) were stirred and heated at 90° C. for 16 h under nitrogen. The solvent was removed under reduced pressure. The residue was purified by silica flash chromatography column, elution gradient from 0 to 10% methanol in DCM. Pure fractions were evaporated to dryness to afford tert-butyl N-[(2S)-3-oxo-2-phenyl-3-[[7-(1H-pyrazol-4-yl)-5H-pyrrolo[3,2-d]pyrimidin-4-yl]amino]propyl]carbamate C-155d (10 mg, 23% yield) as brown solid. LCMS: t_{R}=1.58 min in 3 min chromatography (5-95% MeCN in water (0.02% TFA), waters Acquity UPLC BEH C18 1.7 μm, 2.1*50 mm, 40° C.), MS (ESI) m/z=448.3 [M+H]⁺.

Step 4: To a solution of tert-butyl N-[(2S)-3-oxo-2-phenyl-3-[[7-(1H-pyrazol-4-yl)-5H-pyrrolo[3,2-d]pyrimidin-4-yl]amino]propyl]carbamate C-155d (10 mg, 22.35 μmol) in DCM (0.50 mL) was cooled at 0° C. in ice/water bath, then added trifluoroacetic acid (127.40 mg, 1.12 mmol, 86.08 μL) dropwise into the reaction. The resulting mixture was stirred for 2 h. The reaction mixture was concentrated under reduced pressure. The residue was purified by C18-flash chromatography, elution gradient from 0% to 50% MeCN in water (6 mmol/L NH₄HCO₃). Pure fractions were lyophilized to dryness to afford (2S)-3-amino-2-phenyl-N-[7-(1H-pyrazol-4-yl)-5H-pyrrolo[3,2-d]pyrimidin-4-yl]propanamide Example 155 (2.00 mg, 26% yield) as white solid. LCMS: t_{R}=0.83 min in 3 min chromatography (5-95% MeCN in water (0.02% TFA), waters Acquity UPLC BEH C18 1.7 μm, 2.1*50 mm, 40° C.), MS (ESI) m/z=348.6 [M+H]⁺. ¹H NMR: (400 MHZ, DMSO-d₆) δ 12.85 (s, 1H), 11.06 (s, 1H), 8.56 (s, 1H), 8.18 (s, 2H), 7.97 (s, 1H), 7.42 (d, J=7.3 Hz, 2H), 7.35 (t, J=7.5 Hz, 2H), 7.27 (t, J=7.3 Hz, 1H), 4.07 (dd, J=9.3, 5.1 Hz, 1H), 3.30-3.27 (m, 1H), 2.88 (dd, J=12.5, 5.0 Hz, 1H).

### Example 156

### Preparation of (R)-N-(2-amino-1-phenylethyl)-3-(1H-pyrazol-4-yl)-1H-indole-7-carboxamide

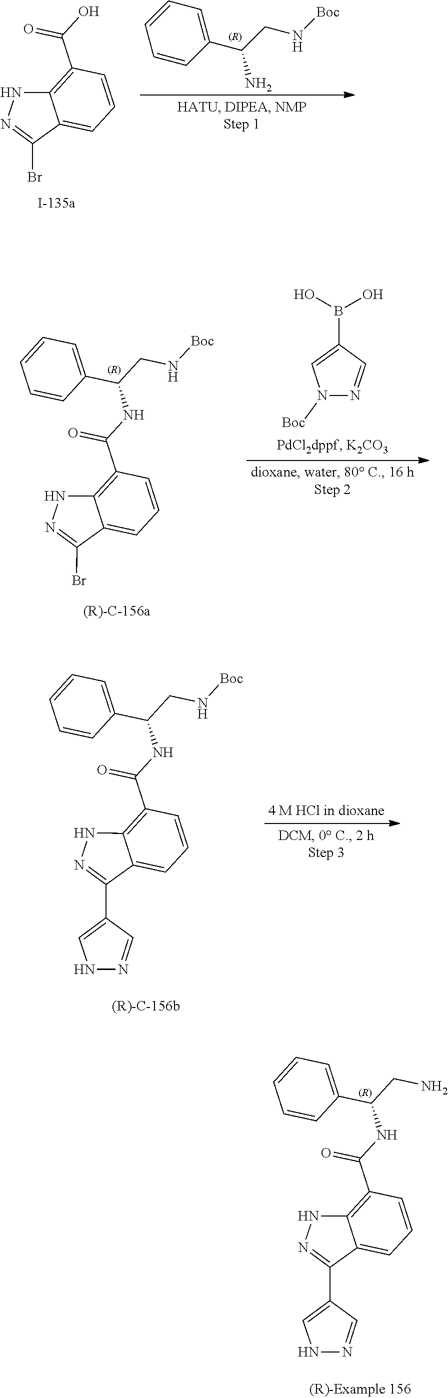

Step 1: To a mixture of acid I-135a (48.20 mg, 199.95 μmol), tert-butyl N-[(2R)-2-amino-2-phenyl-ethyl]carbamate (45.00 mg, 190.43 μmol) and DIPEA (57.81 mg, 571.29 μmol, 79.63 μL) in NMP (2.00 mL) was stirred and cooled at 0° C. by ice/water bath. Then added HATU (86.89 mg, 228.51 μmol) in one portion at this temperature. The resulting mixture was stirred and warmed slowly to room temperature for 2 h. The reaction solution was purified by C18 flash chromatography column, elution gradient from 0 to 50% acetonitrile in water. Pure fractions were evaporated to dryness to afford tert-butyl N-[(2R)-2-[(3-bromo-1H-indazole-7-carbonyl)amino]-2-phenyl-ethyl]carbamate C-156a (60 mg, 69% yield) as yellow solid. LCMS: t_{R}=1.91 min in 3 min chromatography (3 min-5-95% MeCN in water (0.02% TFA), Waters Acquity UPLC BEH C18 1.7 μm, 2.1*50 mm, 40° C.), MS (ESI) m/z=459.0 [M+H]⁺.

Step 2: tert-butyl N-[(2R)-2-[(3-bromo-1H-indazole-7-carbonyl)amino]-2-phenyl-ethyl]carbamate C-156a (60 mg, 130.62 μmol), (1-(tert-Butoxycarbonyl)-1H-pyrazol-4-yl) boronic acid (33.23 mg, 156.75 μmol), Potassium carbonate (54.16 mg, 391.87 μmol, 23.65 μL) and PdCl₂dppf (10.67 mg, 13.06 μmol) in dioxane (1.50 mL) and water (0.30 mL) were stirred and heated at 80° C. for 16 h under nitrogen. The reaction solution was evaporated under reduced pressure. The residue was purified by silica flash chromatography column, elution gradient from 10% to 50% under nitrogen tert-butyl N-[(2R)-2-phenyl-2-[[3-(1H-pyrazol-4-yl)-1H-indazole-7-carbonyl]amino]ethyl]carbamate C-156b (50 mg, 86% yield) as brown solid. LCMS: t_{R}=1.61 min in 3 min chromatography (3 min-5-95% MeCN in water (0.02% TFA), Waters Acquity UPLC BEH C18 1.7 μm, 2.1*50 mm, 40° C.), MS (ESI) m/z=447.3 [M+H]⁺.

Step 3: To a solution of tert-butyl N-[(2R)-2-phenyl-2-[[3-(1H-pyrazol-4-yl)-1H-indazole-7-carbonyl]amino]ethyl]carbamate C-156b (50 mg, 111.98 μmol) in DCM (2.10 mL) was stirred and cooled at 0° C. by ice/water bath. Then added 4 M HCl (102.07 mg, 2.80 mmol) in dioxane (0.70 mL) dropwise into the reaction. The resulting mixture was stirred at this temperature for 2 h. The reaction mixture was concentrated under reduced pressure. The residue was purified by C18 flash chromatography column, elution gradient from 0% to 60% acetronitrile in water (6 mmol/L NH₄HCO₃). Pure fractions were lyophilized to dryness to afford N-[(1R)-2-amino-1-phenyl-ethyl]-3-(1H-pyrazol-4-yl)-1H-indazole-7-carboxamide Example 156 (31 mg, 90% yield) as white solid. LCMS: t_{R}=1.16 min in 3 min chromatography (3 min-5-95% MeCN in water (6 mmol/L NH₄HCO₃), Waters Acquity UPLC BEH C18 1.7 μm, 2.1*50 mm, 40° C.), MS (ESI) m/z=347.2 [M+H]⁺. ¹H NMR: (400 MHz, DMSO-d₆) δ 13.16 (s, 1H), 8.99 (s, 1H), 8.25 (d, J=7.9 Hz, 2H), 8.10 (d, J=7.2 Hz, 1H), 7.41 (d, J=7.2 Hz, 2H), 7.33 (t, J=7.6 Hz, 2H), 7.25 (dt, J=11.9, 7.5 Hz, 2H), 5.05 (s, 1H), 2.94 (ddd, J=18.5, 13.1, 7.1 Hz, 2H).

### Biological Examples

Exemplary compounds disclosed herein have been characterized in one or more of the following biological assays.

### Biological Example 1: IC₅₀ Assay

Recombinant ROCK1 and ROCK2 was purchased from Thermo Fisher with catalogue numbers PV3691 and PR71168. The inhibition potency of compounds against these enzymes was assessed using Lance Ultra Kinase Assay. In brief, recombinant kinases were pre-incubated in the presence or absence of compound at room temperature for 30 minutes. The reaction was initiated by the addition of the ATP (Km) and substrate peptide which could be phosphorylated by kinases in the reaction. After 1 h incubation, the reaction was stopped by the addition of the detection reagent mix containing EDTA. The fluorescence was measured at 615 nm and 665 nm, respectively with excitation wavelength at 320 nm. The calculated signal ratio of 665 nm/615 nm is proportional to the kinase activity. The concentration of compound producing 50% inhibition of the respective kinase (IC₅₀) was calculated using four-parameter logistic.

### Biological Example 2: Cellular PD Assay

PASMC (Human Pulmonary Artery Smooth Muscle Cells) were grown in Smooth Muscle Cell Growth medium (Promocell #C-22162). The cells were resuspended at 3×105 cells per well in 6-well plate in Smooth Muscle Cell Growth medium (Promocell #C-22162) and incubated compound concentration range: (10000/1111/333/111/37/12.3/4.1/0.41/0 nM) for 30 min.

Cells were then placed on ice and washed with ice-cold PBS. Cell lysates were prepared with 2×SDS. Separate proteins by SDS-PAGE 2 hr, transfer of proteins form gel to membrane 90 min, Blocking the membrane with 5% milk 1 hr and incubation in primary antibody overnight at 4° C., Incubate the membrane in HRP-conjugated secondary antibody diluted in blocking buffer (1:2000) for 1 hour at room temperature. Detection using Amersham ECL plus western blotting detection system (LAS4000). The purified product was analysed by SDS PAGE and the amount of target protein calculated by gray scanning by Multi-Gauge V3.0 software.

### Biological Example 3: Cell-Based Phospholipidosis Assay

HepG2 cells were dispensed into Collagen I-coated 96-well black wall/clear bottom assay plates at 5000 cells/90 μL/well.

The assay plates were incubated at 37° C. for 4 h to allow the cells to attach to wells, followed by addition of 30 μL of 1:250 LipidTOX Red dye (1:1000 final, then 120 nL Test compounds were added for the final concentration (50 uM start, 3-fold dilution, 8 doses, in duplicate). The 20 uM of Amiodarone and DMSO (0.1% final) were used for maximum and minimum control.

The assay plates were then incubated at 37° C. for 24 h, 120 μL of 8% paraformaldehyde (4% final) fixative solution containing 8 μg/ml Hoechst 33342 (4 ug/mL final) in DPBS was added to each well. After incubation at room temperature for 25 min, the assay plates were washed with DPBS. The assay plates were sealed and stored at 4° C. before imaging.

The fluorescence intensities (595 nm excitation, 615 nm emission for LipidTOX Red; 352 nm excitation, 461 nm emission for Hoechst 33342) were measured using an High Content Screening System (Operetta, PerkinElmer) with a 10×Fluor objective. Images were acquired for 4 fields in each well and analyzed with the software. Average intensities were subjected to Prism and EC₅₀ were calculated. The present disclosure provided compounds that are potent ROCK1 and ROCK2 inhibitors without liability of inducing phospholipidosis.

The results for some exemplary compounds of the present disclosure are shown in Tables 2-4 below.

TABLE 2
IC₅₀ for ROCK1 and ROCK2 inhibition

| Example | ROCK1 IC₅₀ (nM) | ROCK2 IC₅₀ (nM) |
|---|---|---|
| 1 | 0.59 | 0.27 |
| 3 | 0.33 | 0.17 |
| 5 | 8.0 | 4.3 |
| 6 | 442 | 247 |
| 7 | 34 | 16 |
| 8 | 20 | 11 |
| 9 | 0.24 | 0.14 |
| 10 | 1.6 | 0.63 |
| 14 | 2.2 | 1.1 |
| 15 | 1.4 | 0.74 |
| 16 | 0.84 | 0.37 |
| 17 | 31 | 15 |
| 18 | 0.30 | 0.14 |
| 19 | 2.2 | 0.92 |
| 20 | 2.8 | 1.3 |
| 21 | 125 | 70 |
| 22 | 1.1 | 0.88 |
| 23 | 0.19 | 0.13 |
| 24 | 4.0 | 3.0 |
| 26 | 1.4 | 0.96 |
| 27 | 87 | 66 |
| 29 | 59 | 23 |
| 30 | 3.2 | 1.1 |
| 31 | 1.2 | 0.48 |
| 32 | 0.55 | 0.24 |
| 33 | 97 | 50 |
| 35 | 1.8 | 0.76 |
| 36 | 321 | 174 |
| 37 | 0.71 | 0.43 |
| 38 | 0.44 | 0.26 |
| 39 | 175 | 116 |
| 40 | 0.33 | 0.18 |
| 41 | 5.4 | 3.5 |
| 42 | 0.18 | 0.10 |
| 43 | 1.3 | 0.66 |
| 44 | 111 | 70 |
| 45 | 0.53 | 0.27 |
| 46 | 4.0 | 2.3 |
| 47 | 201 | 121 |
| 48 | 1.6 | 0.84 |
| 49 | 0.86 | 0.65 |
| 50 | 0.32 | 0.22 |
| 51 | 33 | 22 |
| 52 | 0.93 | 0.59 |
| 53 | 5.8 | 4.1 |
| 54 | 0.18 | 0.09 |
| 55 | 2.9 | 1.9 |
| 56 | 1194 | 1098 |
| 57 | 2.7 | 1.6 |
| 58 | 136 | 48 |
| 59 | 2.7 | 0.83 |
| 60 | 2.3 | 0.70 |
| 61 | 26 | 8.9 |
| 62 | 68 | 31 |
| 63 | 0.43 | 0.24 |
| 64 | 0.37 | 0.19 |
| 65 | 4.4 | 2.5 |
| 66 | 604 | 310 |
| 67 | 2.6 | 1.6 |
| 68 | 4.0 | 1.5 |
| 69 | 21 | 9.2 |
| 70 | 2.2 | 0.6 |
| 71 | 227 | 64 |
| 72 | 173 | 67 |
| 73 | 82 | 20 |
| 74 | 2.0 | 0.62 |
| 75 | 204 | 71 |
| 76 | 14 | 7.9 |
| 77 | 84 | 32 |
| 78 | 34 | 16 |
| 79 | 2.9 | 1.7 |
| 80 | 0.39 | 0.18 |
| 81 | 1.8 | 0.91 |
| 82 | 12 | 3.3 |
| 83 | 7.3 | 3.1 |
| 84 | 1.3 | 0.96 |
| 85 | 0.81 | 0.52 |
| 86 | 2.7 | 0.98 |
| 87 | 0.29 | 0.18 |
| 88 | 17 | 9.3 |
| 89 | 1.2 | 0.66 |
| 90 | 5.3 | 0.91 |
| 91 | 1.2 | 0.75 |
| 92 | 16 | 6.0 |
| 93 | 20 | 13 |
| 94 | 6.5 | 3.0 |
| 95 | 3.7 | 2.3 |
| 96 | 7.3 | 4.0 |
| 97 | 122 | 28 |
| 98 | 9.2 | 4.3 |
| 99 | 3.2 | 1.1 |
| 101 | 1.6 | 0.81 |
| 102 | 1.3 | 0.56 |
| 103 | 1.4 | 0.53 |
| 104 | 2.0 | 0.90 |
| 105 | 0.42 | 0.22 |
| 107 | 6.0 | 3.7 |
| 108 | 98 | 51 |
| 109 | 15 | 11 |
| 110 | 6.6 | 3.3 |
| 111 | 297 | 167 |
| 112 | 1.3 | 0.47 |
| 113 | 110 | 43 |
| 121 | 75 | 42 |
| 122 | 11 | 8.9 |
| 126 | 6.3 | 3.0 |
| 127 | 2.7 | 0.99 |
| 128 | 111 | 61 |
| 129 | 1.7 | 1.7 |
| 131 | 22 | 12 |
| 132 | 0.76 | 0.39 |
| 133 | 44 | 31 |
| 134 | 2.6 | 0.88 |
| 135 | 4.5 | 1.2 |
| 136 | 0.62 | 0.24 |
| 137 | 149 | 65 |
| 138 | 73 | 23 |
| 139 | 5.5 | 3.1 |
| 140 | 2.8 | 0.86 |
| 141 | 331 | 163 |
| 142 | 113 | 37 |
| 143 | 397 | 315 |
| 144 | 60 | 32 |
| 145 | 46 | 20 |
| 146 | 18 | 4.9 |
| 147 | 0.88 | 0.40 |
| 148 | 21 | 5.7 |
| 151 | 204 | 71 |
| 155 | 0.96 | 0.63 |

TABLE 3
Cellular Pharmacodynamic Assay Data

| | pMLC2 IC₅₀ (nM) | pMYPT1 IC₅₀ (nM) |
|---|---|---|
| Example | in PASMC | in PASMC |
| Netasudil-M1 | 14 | 22 |
| 3 | 46 | 99 |
| 7 | 13 | — |
| 9 | 15 | 2.2 |
| 10 | 8.1 | 10 |
| 18 | 4.2 | 5.6 |
| 20 | 38 | 41 |
| 22 | 13 | 1.5 |
| 23 | 3.5 | 4.3 |
| 26 | 85 | 153 |
| 31 | 31 | — |
| 32 | 42 | 62 |
| 37 | 8 | 6.2 |
| 38 | 8.2 | 13 |
| 42 | 3 | 2.8 |
| 43 | 102 | 18 |
| 45 | 29 | 11 |
| 52 | 12 | — |
| 59 | 61 | 155 |
| 105 | 95 | — |
| 135 | 1.3 | 26 |
| 136 | 5.1 | 1.9 |
| 154 | 8.3 | — |

TABLE 4
Cell-based Phospholipidosis Assay Data

| Example | EC₅₀ (uM) |
|---|---|
| Amiodarone | 1.37 |
| Netarsudil | 0.89 |
| 3 | >200 |
| 10 | >66.6 |
| 18 | 10.9 |
| 19 | >50 |
| 20 | >200 |
| 26 | >200 |
| 31 | >50 |
| 32 | >200 |
| 38 | 6.23 |
| 42 | >200 |
| 49 | >200 |
| 59 | >66.6 |
| 60 | >50 |
| 74 | >200 |
| 91 | 24.4 |
| 105 | 23.6 |

The foregoing description is considered as illustrative only of the principles of the present disclosure. Further, since numerous modifications and changes will be readily apparent to those skilled in the art, it is not desired to limit the invention to the exact construction and process shown as described above. Accordingly, all suitable modifications and equivalents may be considered to fall within the scope of the invention as defined by the claims that follow.

1. A compound of formula I 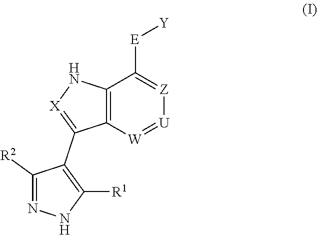
or a pharmaceutically acceptable salt thereof, wherein:
each of R¹ and R² is independently selected from the group consisting of hydrogen, hydroxy, halogen, cyano, amino, alkyl, alkenyl, alkynyl, heteroalkyl, heteroalkenyl, and heteroalkynyl, wherein the alkyl, alkenyl, alkynyl, heteroalkyl, heteroalkenyl, and heteroalkynyl are each optionally substituted with one or more groups independently selected from hydroxyl, halogen, cyano or amino;
each of X, W, Z and U is independently N or C(R³);
R³ is selected from the group consisting of hydrogen, hydroxy, halogen, cyano, amino, alkyl, alkenyl, alkynyl, heteroalkyl, heteroalkenyl, and heteroalkynyl, wherein the alkyl, alkenyl, alkynyl, heteroalkyl, heteroalkenyl, and heteroalkynyl are each optionally substituted with one or more groups independently selected from hydroxyl, halogen, cyano or amino;
E is —N(R⁴)C(═O)—, —C(═O)N(R⁴)—, —N(R⁴)SO₂—, or —SO₂N(R⁴)—;
R⁴ is selected from the group consisting of hydrogen, alkyl, alkenyl, alkynyl, heteroalkyl, heteroalkenyl, and heteroalkynyl;
Y is —Y¹—Y²—(Y³)ₙ, wherein
Y¹ is null or —C(R⁵)₂—,
each R⁵ is independently selected from the group consisting of hydrogen, —N(R^{a})₂, alkyl, alkenyl, alkynyl, heteroalkyl, heteroalkenyl, heteroalkynyl, cycloalkyl, heterocyclyl, aryl, heteroaryl, and -alkyl-heterocyclyl, wherein the alkyl, alkenyl, alkynyl, heteroalkyl, heteroalkenyl, heteroalkynyl, cycloalkyl, heterocyclyl, aryl, heteroaryl and -alkyl-heterocyclyl are optionally substituted with one or more R⁶,
each R⁶ is independently selected from the group consisting of hydroxy, halogen, cyano, amino, alkyl, alkenyl, alkynyl, heteroalkyl, heteroalkenyl, heteroalkynyl, and -alkyl-N(R^{a}) 2;
each R^{a} is independently selected from hydrogen or alkyl;
Y² is selected from —(CH₂)ₚ-cycloalkyl-*, —(CH₂)ₚ-heterocyclyl-*, —(CH₂)ₚ-aryl-*, or —(CH₂)ₚ-heteroaryl-*, each of which can be optionally substituted with one or more groups independently selected from hydroxy, halogen, cyano, amino, alkyl, alkenyl, alkynyl, heteroalkyl, heteroalkenyl, or heteroalkynyl, and wherein p is 0, 1 or 2, and * end of Y² is connected to Y³;
Y³ is null or —Y³¹—Y³²—Y³³; wherein
Y³¹ is selected from null, alkyl, alkenyl or alkynyl;
Y³² is selected from null, —O—#, —OC(—O)—#, —C(—O)O—#, —P(═O)(R^{b})—#, —OC(═O)N(R^{b})—#, —N(R^{b})C(═O)—#, or —C(═O)N(R^{b})—#, wherein each R^{b} is independently selected from hydrogen or alkyl, and #end of Y³² is connected to Y³³;
Y³³ is selected from a group consisting of hydrogen, hydroxyl, cyano, halogen, —N(R^{c})₂, alkyl, alkenyl, alkynyl, heteroalkyl, heteroalkenyl, heteroalkynyl, cycloalkyl, heterocyclyl, aryl, and heteroaryl, wherein the alkyl, alkenyl, alkynyl, heteroalkyl, heteroalkenyl, heteroalkynyl, cycloalkyl, heterocyclyl, aryl, and heteroaryl are optionally substituted with one or more R⁷, wherein each R⁷ is independently selected from halogen, hydroxy, amino, cyano, nitrooxy, alkyl, alkenyl, alkynyl, heteroalkyl, heteralkenyl, heteroalkynyl, or haloalkyl, and each R^{c} is independently selected from hydrogen or alkyl;
and
n is an integer from 1 to 5.

2. The compound of claim 1, or a pharmaceutically acceptable salt thereof, wherein each of R¹ and R² is independently hydrogen, halogen, hydrogen, cyano, amino, or alkyl optionally substituted with one or more halogens.

3. The compound of claim 2, or a pharmaceutically acceptable salt thereof, wherein R¹ is;
hydrogen or alkyl, and R² is hydrogen, halogen or alkyl; or
R¹ and R² are both hydrogen; or
R¹ and R² are both alkyl; or,
R¹ is methyl, and R² is halogen; or
R¹ is hydrogen, and R² is halogen; or
R¹ is hydrogen, and R² is fluoro; or
R¹ is hydrogen, and R² is alkyl; or
R¹ is hydrogen, and R² is methyl.

4-10. (canceled)

11. The compound of claim 1, or a pharmaceutically acceptable salt thereof, wherein,
X, W, U and Z are C(R³), or
X and U are C(R³), W and Z are N.

12. (canceled)

13. The compound of claim 11, or a pharmaceutically acceptable salt thereof, wherein R³ is hydrogen.

14. The compound of claim 1, or a pharmaceutically acceptable salt thereof, wherein E is —N(R⁴)C(═O)— or —C(═O)N(R⁴)—.

15. The compound of claim 14, or a pharmaceutically acceptable salt thereof, wherein R⁴ is hydrogen.

16. (canceled)

17. (canceled)

18. The compound of claim 1, or a pharmaceutically acceptable salt thereof, wherein one R⁵ is hydrogen, the other R⁵ is selected from —N(R^{a})₂, alkyl, cycloalkyl, heterocyclyl or -alkyl-heterocyclyl, wherein the alkyl, cycloalkyl, heterocyclyl and -alkyl-heterocyclyl are optionally substituted with one or more R⁶.

19. The compound of claim 1, or a pharmaceutically acceptable salt thereof, wherein one R⁵ is alkyl, the other R⁵ is selected from —N(R^{a})₂, alkyl, cycloalkyl, or heterocyclyl, wherein the alkyl, cycloalkyl, and heterocyclyl are optionally substituted with one or more R⁶.

20. The compound of claim 18, or a pharmaceutically acceptable salt thereof, wherein R⁶ is selected from amino, alkyl or -alkyl-N(R^{a})₂.

21. The compound of claim 1, or a pharmaceutically acceptable salt thereof, wherein Y¹ is selected from the group consisting of:

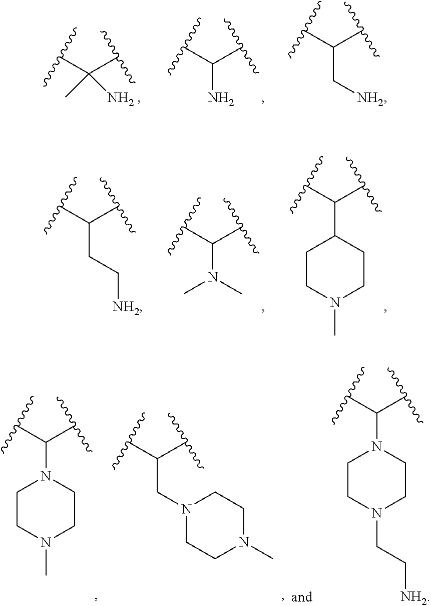

22. The compound of claim 1, or a pharmaceutically acceptable salt thereof, wherein Y² is:
—(CH₂)ₚ-cycloalkyl-*; or
Y² is —(CH₂)ₚ—(C₃₋₁₀ cycloalkyl)-*, and p is 0 or 1; or
Y² is cyclopentyl or cyclohexyl; or
Y² is —(CH₂)ₚ-heterocyclyl-*; or
Y² is —(CH₂)ₚ-(5- to 12-membered heterocyclyl)-*, and p is 0 or 1; or
Y² is tetrahydrofuranyl, pyrrolidinyl, or tetrahydropyranyl; or
Y² is —(CH₂)ₚ-aryl-*; or
Y² is —(CH₂)ₚ—(C₅₋₁₂ aryl)-*, and p is 0 or 1; or
Y² is phenyl or —CH₂-phenyl-*; or
Y² is —(CH₂)ₚ-heteroaryl-*; or
Y² is —(CH₂)ₚ-(5- to 12-membered heteroaryl)-*, and p is 0 or 1; or
Y² is pyridinyl, tetrahydroisoquinolinyl, —CH₂-imidazolyl-* or —CH₂-indolyl-*.

23-33. (canceled)

34. The compound of claim 1, or a pharmaceutically acceptable salt thereof, wherein Y³¹ is null or alkyl.

35. (canceled)

36. The compound of claim 1, or a pharmaceutically acceptable salt thereof, wherein Y³² is selected from null, —O—#, —C(═O)O—#, —P(═O)(R^{b})—#, —N(R^{b})C(═O)—#, or —C(═O)N(R^{b})—#.

37. The compound of claim 1, or a pharmaceutically acceptable salt thereof, wherein Y³³ is selected from hydrogen, hydroxyl, cyano, halogen, —N(R^{c})₂, alkyl, or heteroalkyl, wherein the alkyl and heteroalkyl are optionally substituted with one or more R⁷.

38. The compound of claim 37, or a pharmaceutically acceptable salt thereof, wherein Y³³ is selected from hydrogen, hydroxyl, cyano, halogen, —NH₂, methyl, —CH₂CH₂OCH₃.

39. The compound of claim 1, or a pharmaceutically acceptable salt thereof, wherein Y³¹ is alkyl.

40. The compound of claim 38, or a pharmaceutically acceptable salt thereof, wherein Y³² is selected from null, —O—#, —OC(═O)—#, —OC(═O)N(R^{b})—#, or —N(R^{b})C(═O)—#.

41. The compound of claim 38, or a pharmaceutically acceptable salt thereof, wherein Y³³ is selected from —N(R^{c})₂, alkyl, or aryl, wherein the alkyl and aryl are optionally substituted with one or more R⁷.

42. The compound of claim 41, or a pharmaceutically acceptable salt thereof, wherein each R⁷ is independently selected from halogen, hydroxy, amino, cyano, nitrooxy, or alkyl.

43. The compound of claim 42, or a pharmaceutically acceptable salt thereof, wherein Y³³ is selected from —NH₂, —N(CH₃)₂, methyl, dimethylphenyl, or nitrooxypentyl.

44. The compound of claim 1, or a pharmaceutically acceptable salt thereof, wherein n is 1 or 2.

45. The compound of claim 1, or a pharmaceutically acceptable salt thereof, wherein the compound is selected from the group consisting of:

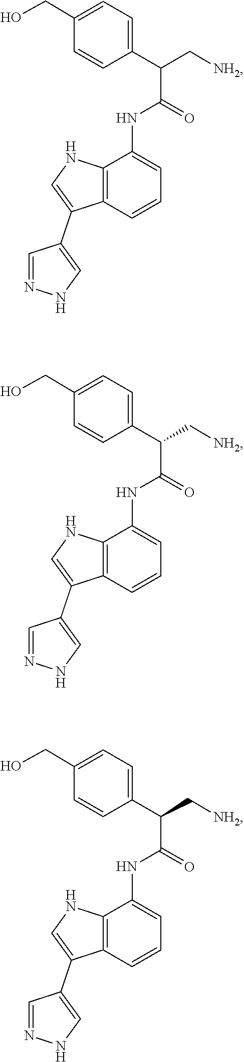 [SMILES: NCC(C(=O)Nc1cccc2c(-c3cn[nH]c3)c[nH]c12)c1ccc(CO)cc1 ; NC[C@H](C(=O)Nc1cccc2c(-c3cn[nH]c3)c[nH]c12)c1ccc(CO)cc1 ; NC[C@@H](C(=O)Nc1cccc2c(-c3cn[nH]c3)c[nH]c12)c1ccc(CO)cc1]

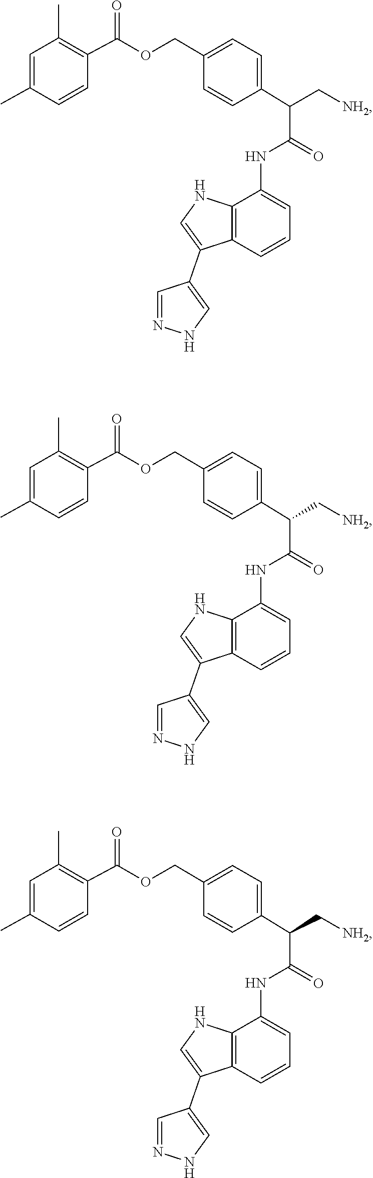 [SMILES: Cc1ccc(C(=O)OCc2ccc(C(CN)C(=O)Nc3cccc4c(-c5cn[nH]c5)c[nH]c34)cc2)c(C)c1 ; Cc1ccc(C(=O)OCc2ccc([C@H](CN)C(=O)Nc3cccc4c(-c5cn[nH]c5)c[nH]c34)cc2)c(C)c1 ; Cc1ccc(C(=O)OCc2ccc([C@@H](CN)C(=O)Nc3cccc4c(-c5cn[nH]c5)c[nH]c34)cc2)c(C)c1]

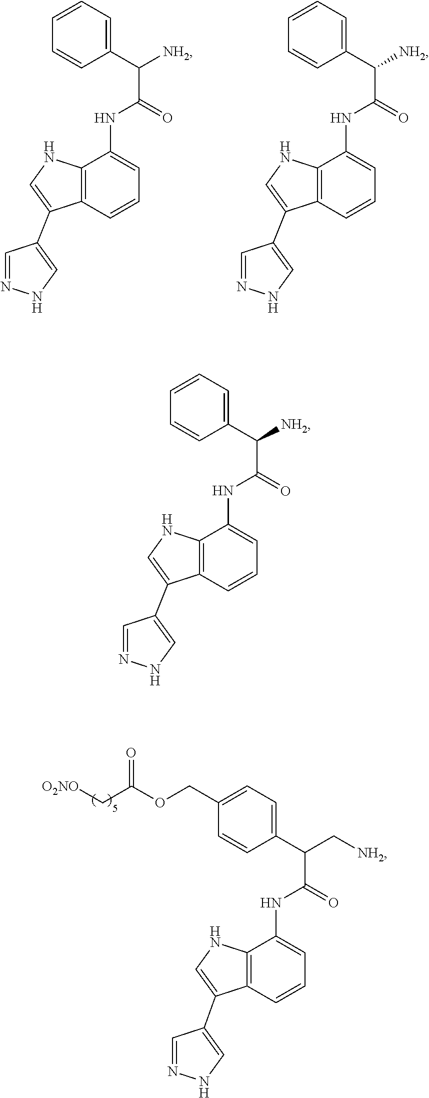

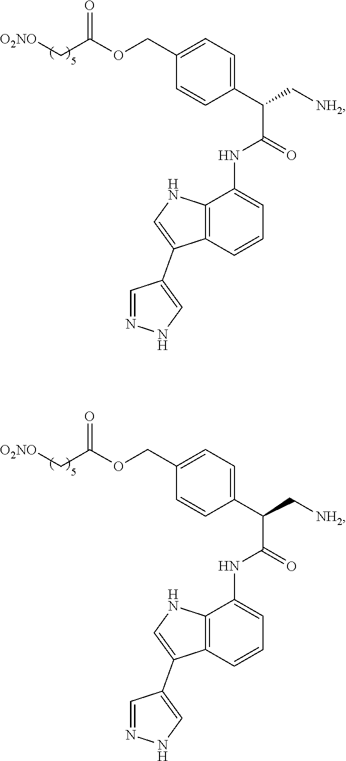

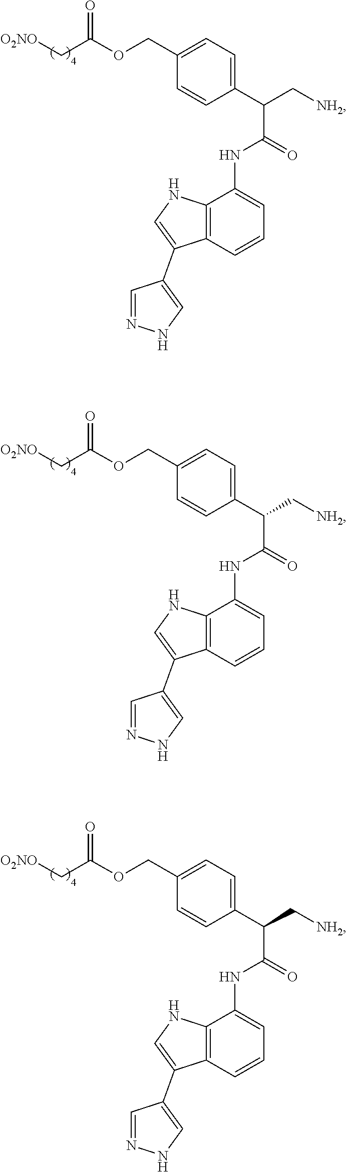

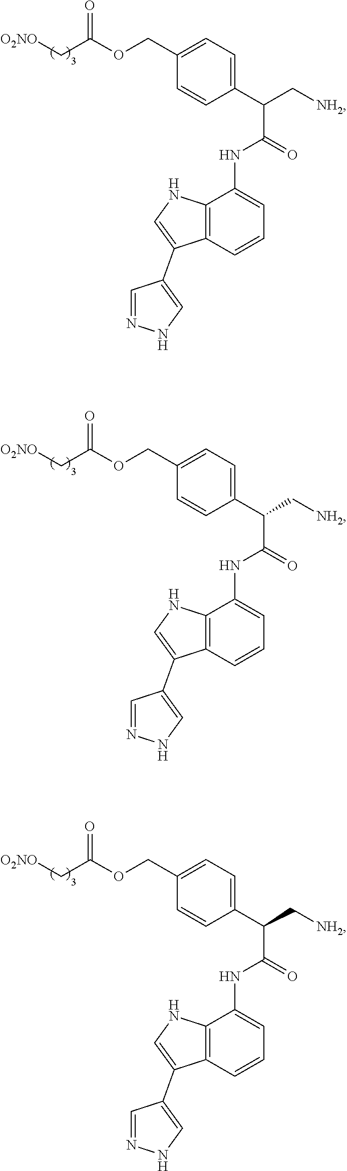

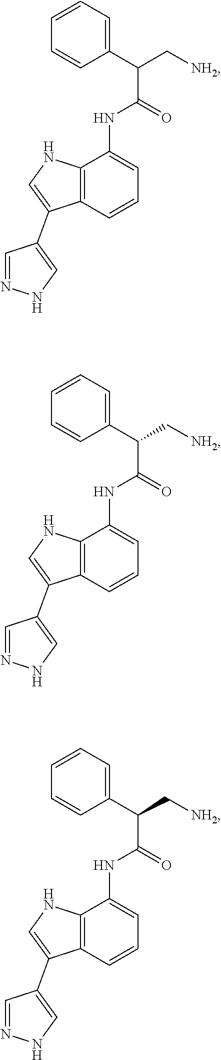 [SMILES: NCC(C(=O)Nc1cccc2c(-c3cn[nH]c3)c[nH]c12)c1ccccc1 ; NC[C@H](C(=O)Nc1cccc2c(-c3cn[nH]c3)c[nH]c12)c1ccccc1 ; NC[C@@H](C(=O)Nc1cccc2c(-c3cn[nH]c3)c[nH]c12)c1ccccc1]

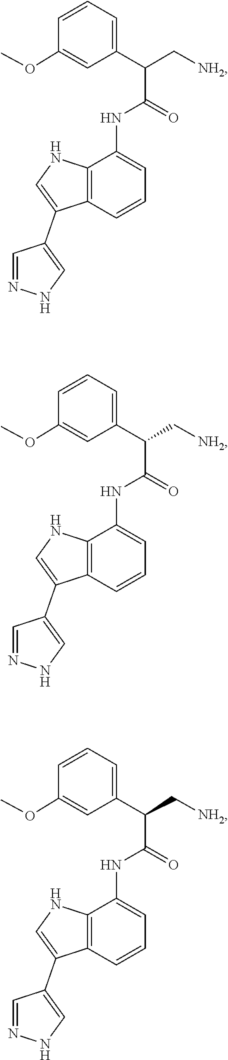 [SMILES: COc1cccc(C(CN)C(=O)Nc2cccc3c(-c4cn[nH]c4)c[nH]c23)c1 ; COc1cccc([C@H](CN)C(=O)Nc2cccc3c(-c4cn[nH]c4)c[nH]c23)c1 ; COc1cccc([C@@H](CN)C(=O)Nc2cccc3c(-c4cn[nH]c4)c[nH]c23)c1]

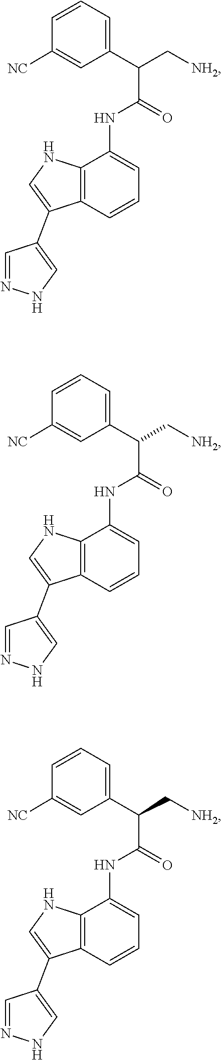 [SMILES: N#Cc1cccc(C(CN)C(=O)Nc2cccc3c(-c4cn[nH]c4)c[nH]c23)c1 ; N#Cc1cccc([C@H](CN)C(=O)Nc2cccc3c(-c4cn[nH]c4)c[nH]c23)c1 ; N#Cc1cccc([C@@H](CN)C(=O)Nc2cccc3c(-c4cn[nH]c4)c[nH]c23)c1]

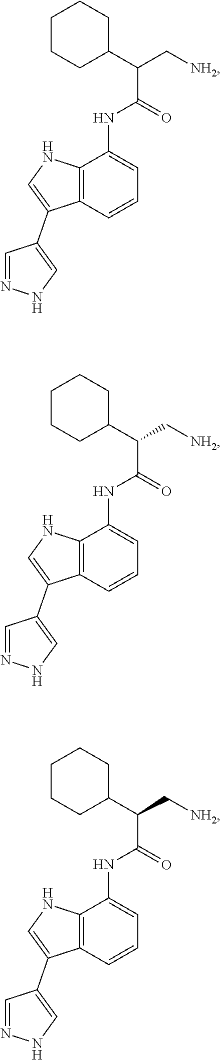 [SMILES: NCC(C(=O)Nc1cccc2c(-c3cn[nH]c3)c[nH]c12)C1CCCCC1 ; NC[C@H](C(=O)Nc1cccc2c(-c3cn[nH]c3)c[nH]c12)C1CCCCC1 ; NC[C@@H](C(=O)Nc1cccc2c(-c3cn[nH]c3)c[nH]c12)C1CCCCC1]

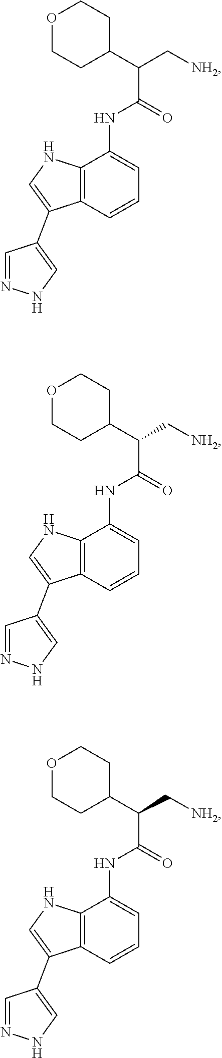 [SMILES: NCC(C(=O)Nc1cccc2c(-c3cn[nH]c3)c[nH]c12)C1CCOCC1 ; NC[C@H](C(=O)Nc1cccc2c(-c3cn[nH]c3)c[nH]c12)C1CCOCC1 ; NC[C@@H](C(=O)Nc1cccc2c(-c3cn[nH]c3)c[nH]c12)C1CCOCC1]

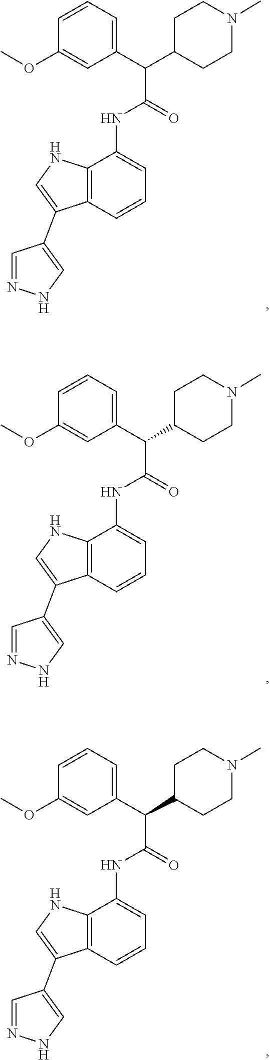

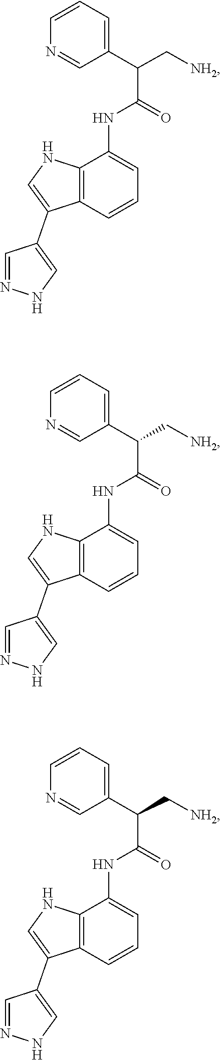 [SMILES: NCC(C(=O)Nc1cccc2c(-c3cn[nH]c3)c[nH]c12)c1cccnc1 ; NC[C@H](C(=O)Nc1cccc2c(-c3cn[nH]c3)c[nH]c12)c1cccnc1 ; NC[C@@H](C(=O)Nc1cccc2c(-c3cn[nH]c3)c[nH]c12)c1cccnc1]

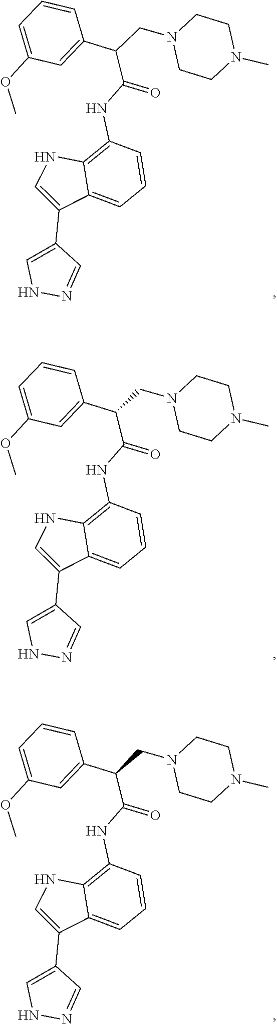 [SMILES: COc1cccc(C(CN2CCN(C)CC2)C(=O)Nc2cccc3c(-c4cn[nH]c4)c[nH]c23)c1 ; COc1cccc([C@H](CN2CCN(C)CC2)C(=O)Nc2cccc3c(-c4cn[nH]c4)c[nH]c23)c1 ; COc1cccc([C@@H](CN2CCN(C)CC2)C(=O)Nc2cccc3c(-c4cn[nH]c4)c[nH]c23)c1]

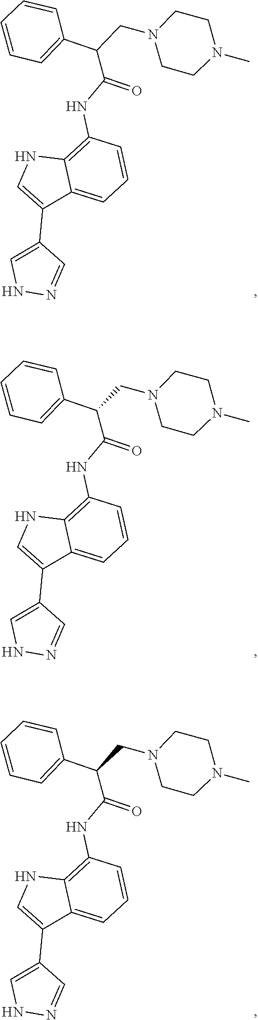 [SMILES: CN1CCN(CC(C(=O)Nc2cccc3c(-c4cn[nH]c4)c[nH]c23)c2ccccc2)CC1 ; CN1CCN(C[C@H](C(=O)Nc2cccc3c(-c4cn[nH]c4)c[nH]c23)c2ccccc2)CC1 ; CN1CCN(C[C@@H](C(=O)Nc2cccc3c(-c4cn[nH]c4)c[nH]c23)c2ccccc2)CC1]

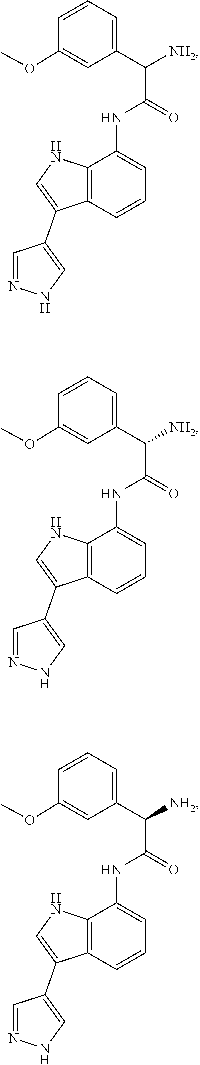 [SMILES: COc1cccc(C(N)C(=O)Nc2cccc3c(-c4cn[nH]c4)c[nH]c23)c1 ; COc1cccc([C@H](N)C(=O)Nc2cccc3c(-c4cn[nH]c4)c[nH]c23)c1 ; COc1cccc([C@@H](N)C(=O)Nc2cccc3c(-c4cn[nH]c4)c[nH]c23)c1]

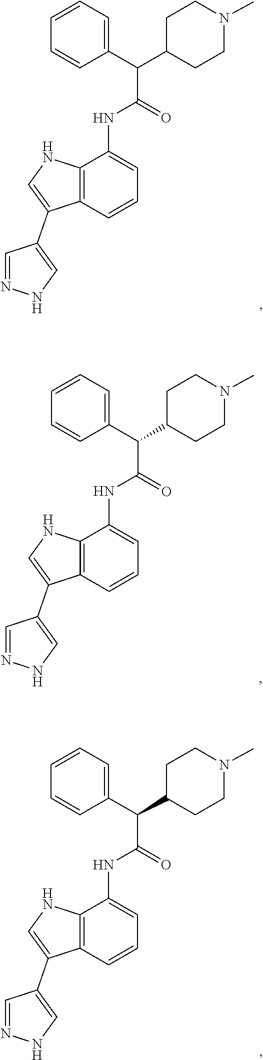 [SMILES: CN1CCC(C(C(=O)Nc2cccc3c(-c4cn[nH]c4)c[nH]c23)c2ccccc2)CC1 ; CN1CCC([C@H](C(=O)Nc2cccc3c(-c4cn[nH]c4)c[nH]c23)c2ccccc2)CC1 ; CN1CCC([C@@H](C(=O)Nc2cccc3c(-c4cn[nH]c4)c[nH]c23)c2ccccc2)CC1]

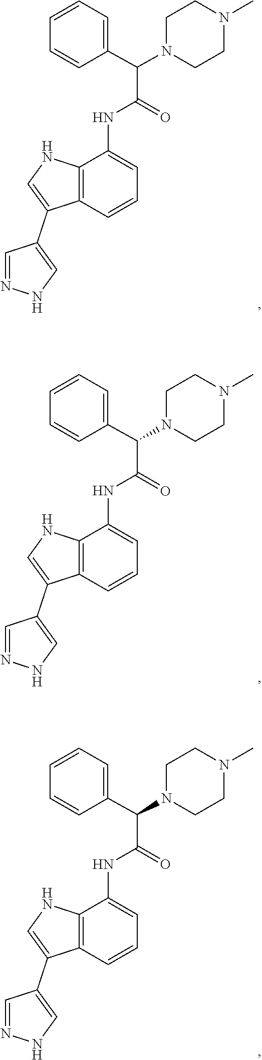 [SMILES: CN1CCN(C(C(=O)Nc2cccc3c(-c4cn[nH]c4)c[nH]c23)c2ccccc2)CC1 ; CN1CCN([C@H](C(=O)Nc2cccc3c(-c4cn[nH]c4)c[nH]c23)c2ccccc2)CC1 ; CN1CCN([C@@H](C(=O)Nc2cccc3c(-c4cn[nH]c4)c[nH]c23)c2ccccc2)CC1]

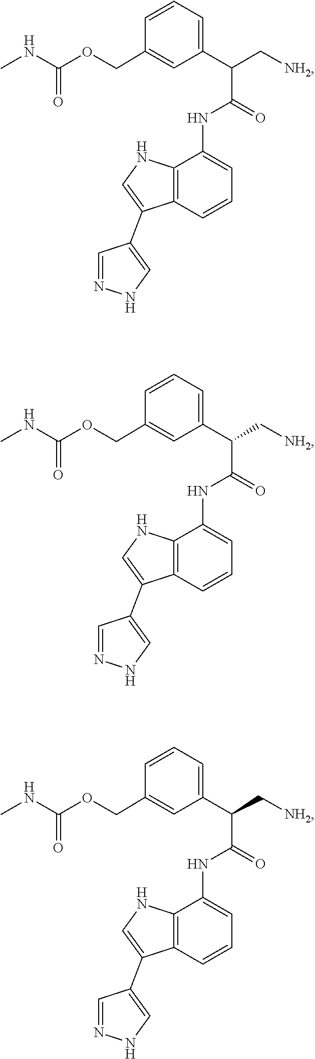 [SMILES: CNC(=O)OCc1cccc(C(CN)C(=O)Nc2cccc3c(-c4cn[nH]c4)c[nH]c23)c1 ; CNC(=O)OCc1cccc([C@H](CN)C(=O)Nc2cccc3c(-c4cn[nH]c4)c[nH]c23)c1 ; CNC(=O)OCc1cccc([C@@H](CN)C(=O)Nc2cccc3c(-c4cn[nH]c4)c[nH]c23)c1]

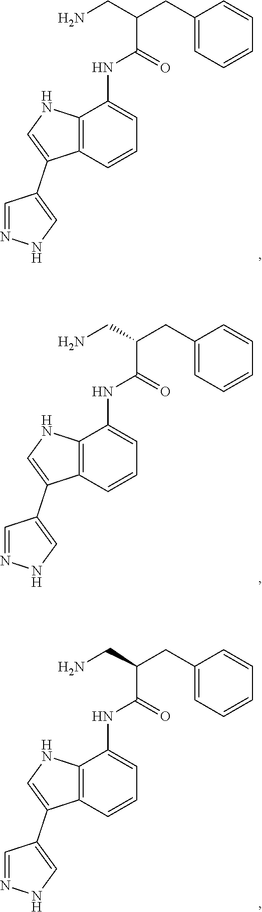 [SMILES: NCC(Cc1ccccc1)C(=O)Nc1cccc2c(-c3cn[nH]c3)c[nH]c12 ; NC[C@H](Cc1ccccc1)C(=O)Nc1cccc2c(-c3cn[nH]c3)c[nH]c12 ; NC[C@@H](Cc1ccccc1)C(=O)Nc1cccc2c(-c3cn[nH]c3)c[nH]c12]

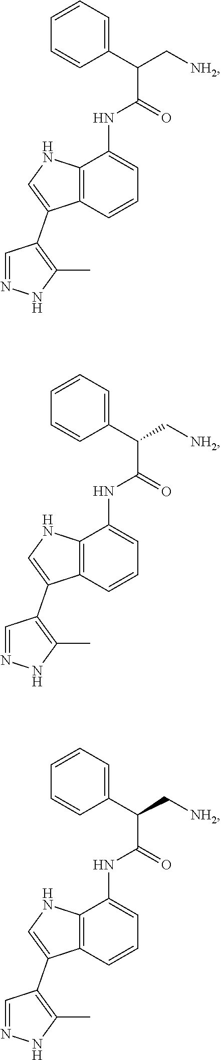 [SMILES: Cc1[nH]ncc1-c1c[nH]c2c(NC(=O)C(CN)c3ccccc3)cccc12 ; Cc1[nH]ncc1-c1c[nH]c2c(NC(=O)[C@@H](CN)c3ccccc3)cccc12 ; Cc1[nH]ncc1-c1c[nH]c2c(NC(=O)[C@H](CN)c3ccccc3)cccc12]

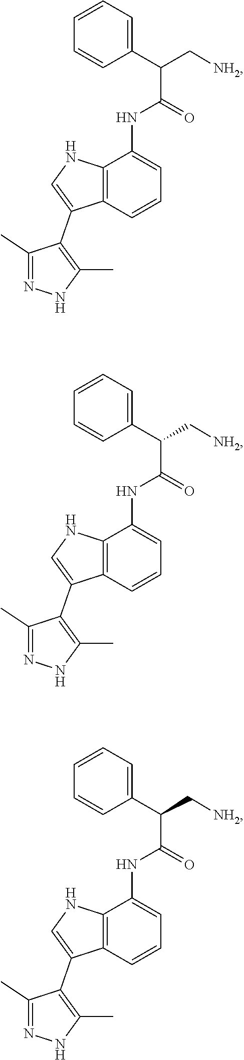 [SMILES: Cc1n[nH]c(C)c1-c1c[nH]c2c(NC(=O)C(CN)c3ccccc3)cccc12 ; Cc1n[nH]c(C)c1-c1c[nH]c2c(NC(=O)[C@@H](CN)c3ccccc3)cccc12 ; Cc1n[nH]c(C)c1-c1c[nH]c2c(NC(=O)[C@H](CN)c3ccccc3)cccc12]

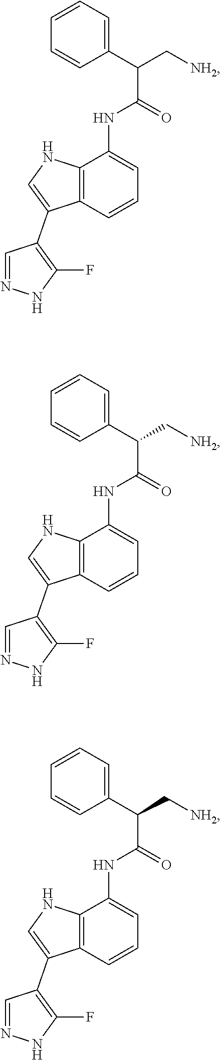 [SMILES: NCC(C(=O)Nc1cccc2c(-c3cn[nH]c3F)c[nH]c12)c1ccccc1 ; NC[C@H](C(=O)Nc1cccc2c(-c3cn[nH]c3F)c[nH]c12)c1ccccc1 ; NC[C@@H](C(=O)Nc1cccc2c(-c3cn[nH]c3F)c[nH]c12)c1ccccc1]

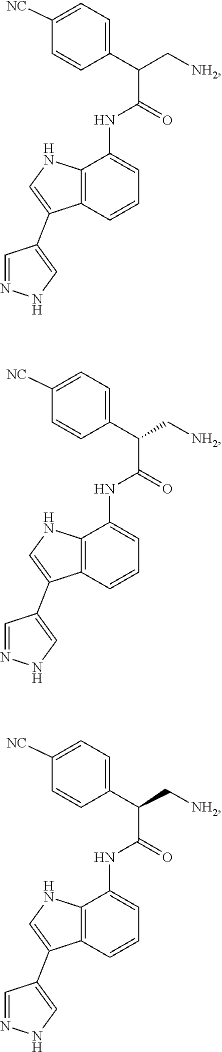 [SMILES: N#Cc1ccc(C(CN)C(=O)Nc2cccc3c(-c4cn[nH]c4)c[nH]c23)cc1 ; N#Cc1ccc([C@H](CN)C(=O)Nc2cccc3c(-c4cn[nH]c4)c[nH]c23)cc1 ; N#Cc1ccc([C@@H](CN)C(=O)Nc2cccc3c(-c4cn[nH]c4)c[nH]c23)cc1]

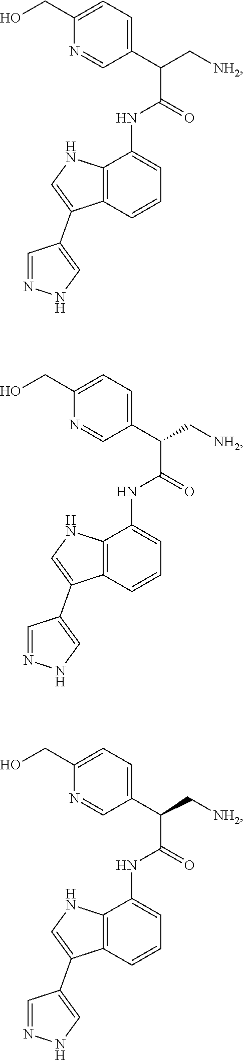 [SMILES: NCC(C(=O)Nc1cccc2c(-c3cn[nH]c3)c[nH]c12)c1ccc(CO)nc1 ; NC[C@H](C(=O)Nc1cccc2c(-c3cn[nH]c3)c[nH]c12)c1ccc(CO)nc1 ; NC[C@@H](C(=O)Nc1cccc2c(-c3cn[nH]c3)c[nH]c12)c1ccc(CO)nc1]

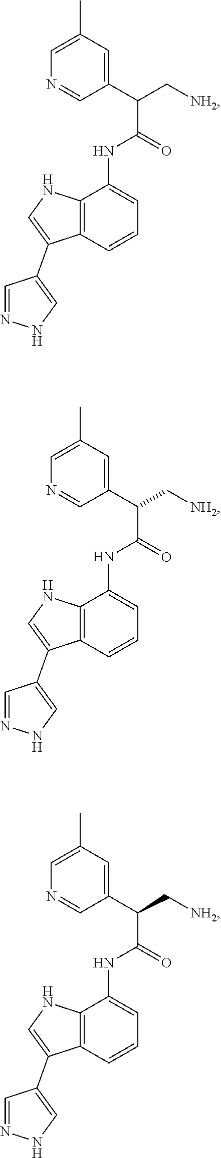 [SMILES: Cc1cncc(C(CN)C(=O)Nc2cccc3c(-c4cn[nH]c4)c[nH]c23)c1 ; Cc1cncc([C@H](CN)C(=O)Nc2cccc3c(-c4cn[nH]c4)c[nH]c23)c1 ; Cc1cncc([C@@H](CN)C(=O)Nc2cccc3c(-c4cn[nH]c4)c[nH]c23)c1]

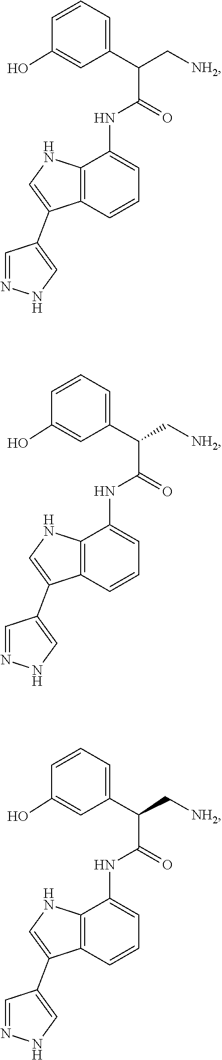 [SMILES: NCC(C(=O)Nc1cccc2c(-c3cn[nH]c3)c[nH]c12)c1cccc(O)c1 ; NC[C@H](C(=O)Nc1cccc2c(-c3cn[nH]c3)c[nH]c12)c1cccc(O)c1 ; NC[C@@H](C(=O)Nc1cccc2c(-c3cn[nH]c3)c[nH]c12)c1cccc(O)c1]

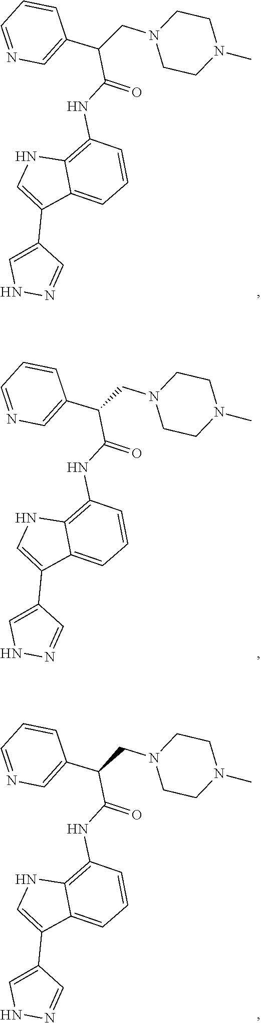 [SMILES: CN1CCN(CC(C(=O)Nc2cccc3c(-c4cn[nH]c4)c[nH]c23)c2cccnc2)CC1 ; CN1CCN(C[C@H](C(=O)Nc2cccc3c(-c4cn[nH]c4)c[nH]c23)c2cccnc2)CC1 ; CN1CCN(C[C@@H](C(=O)Nc2cccc3c(-c4cn[nH]c4)c[nH]c23)c2cccnc2)CC1]

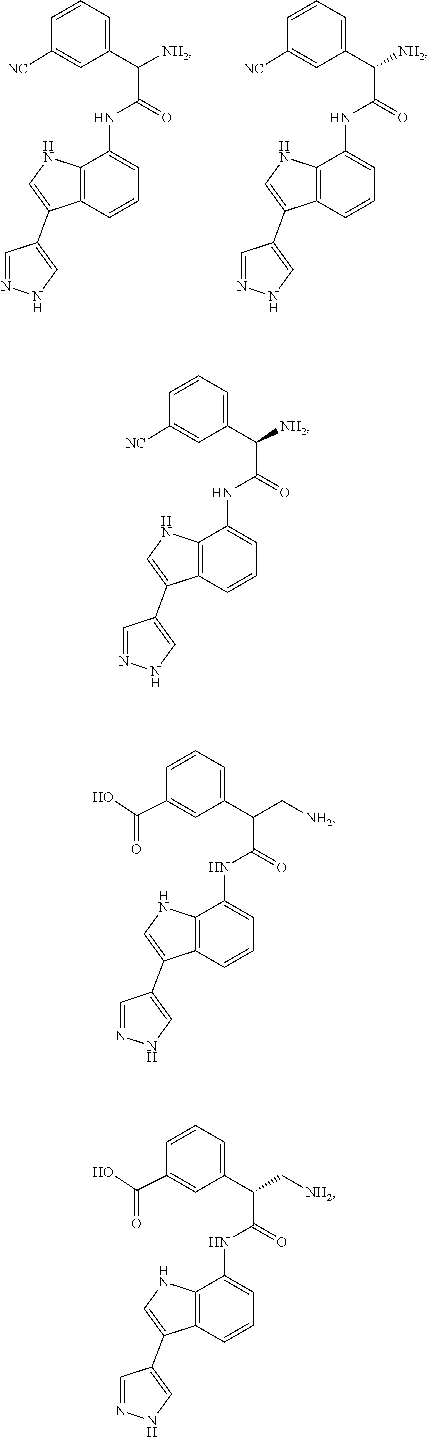 [SMILES: N#Cc1cccc(C(N)C(=O)Nc2cccc3c(-c4cn[nH]c4)c[nH]c23)c1 ; N#Cc1cccc([C@H](N)C(=O)Nc2cccc3c(-c4cn[nH]c4)c[nH]c23)c1 ; N#Cc1cccc([C@@H](N)C(=O)Nc2cccc3c(-c4cn[nH]c4)c[nH]c23)c1 ; NCC(C(=O)Nc1cccc2c(-c3cn[nH]c3)c[nH]c12)c1cccc(C(=O)O)c1 ; NC[C@H](C(=O)Nc1cccc2c(-c3cn[nH]c3)c[nH]c12)c1cccc(C(=O)O)c1]

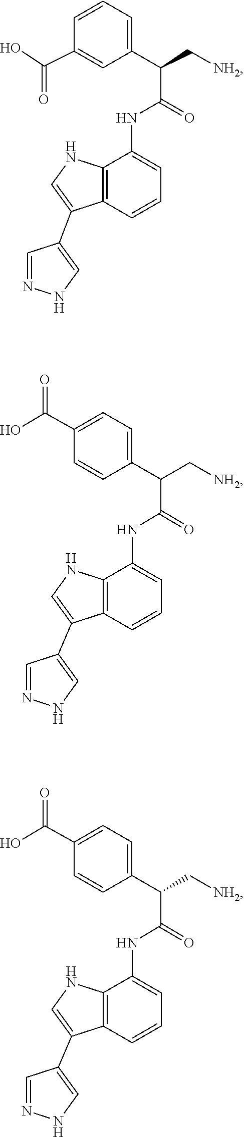 [SMILES: NC[C@@H](C(=O)Nc1cccc2c(-c3cn[nH]c3)c[nH]c12)c1cccc(C(=O)O)c1 ; NCC(C(=O)Nc1cccc2c(-c3cn[nH]c3)c[nH]c12)c1ccc(C(=O)O)cc1 ; NC[C@H](C(=O)Nc1cccc2c(-c3cn[nH]c3)c[nH]c12)c1ccc(C(=O)O)cc1]

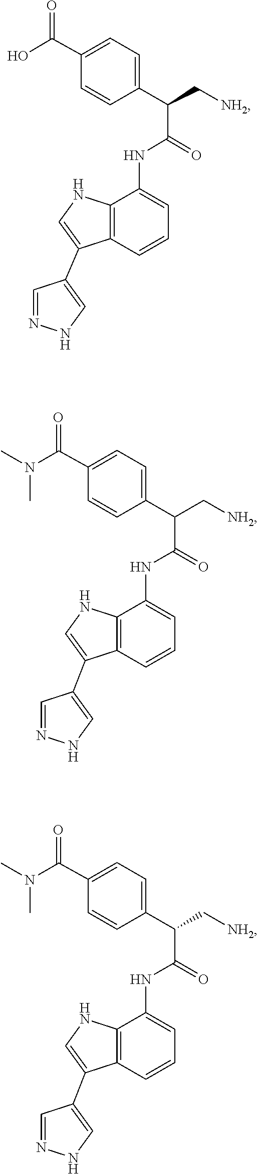 [SMILES: NC[C@@H](C(=O)Nc1cccc2c(-c3cn[nH]c3)c[nH]c12)c1ccc(C(=O)O)cc1 ; CN(C)C(=O)c1ccc(C(CN)C(=O)Nc2cccc3c(-c4cn[nH]c4)c[nH]c23)cc1 ; CN(C)C(=O)c1ccc([C@H](CN)C(=O)Nc2cccc3c(-c4cn[nH]c4)c[nH]c23)cc1]

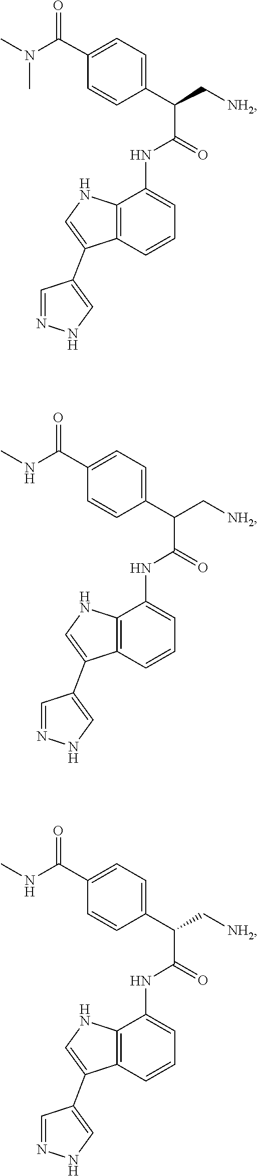 [SMILES: CN(C)C(=O)c1ccc([C@@H](CN)C(=O)Nc2cccc3c(-c4cn[nH]c4)c[nH]c23)cc1 ; CNC(=O)c1ccc(C(CN)C(=O)Nc2cccc3c(-c4cn[nH]c4)c[nH]c23)cc1 ; CNC(=O)c1ccc([C@H](CN)C(=O)Nc2cccc3c(-c4cn[nH]c4)c[nH]c23)cc1]

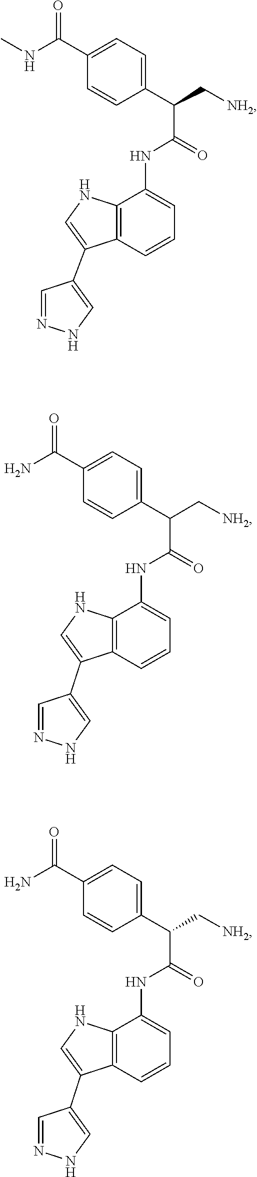 [SMILES: CNC(=O)c1ccc([C@@H](CN)C(=O)Nc2cccc3c(-c4cn[nH]c4)c[nH]c23)cc1 ; NCC(C(=O)Nc1cccc2c(-c3cn[nH]c3)c[nH]c12)c1ccc(C(N)=O)cc1 ; NC[C@H](C(=O)Nc1cccc2c(-c3cn[nH]c3)c[nH]c12)c1ccc(C(N)=O)cc1]

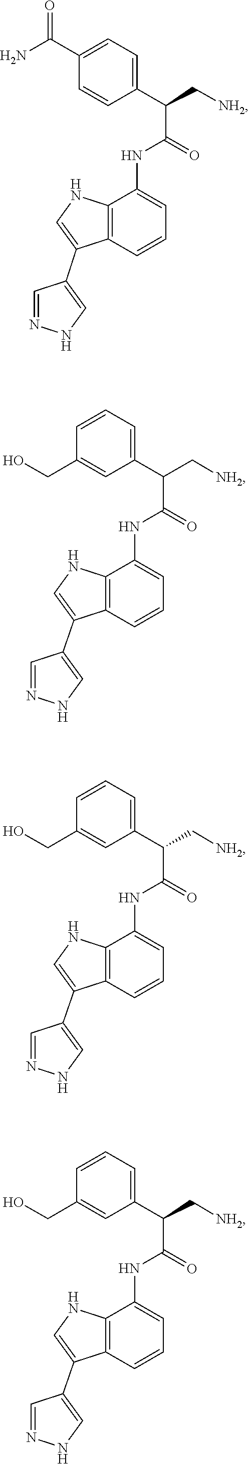 [SMILES: NC[C@@H](C(=O)Nc1cccc2c(-c3cn[nH]c3)c[nH]c12)c1ccc(C(N)=O)cc1 ; NCC(C(=O)Nc1cccc2c(-c3cn[nH]c3)c[nH]c12)c1cccc(CO)c1 ; NC[C@H](C(=O)Nc1cccc2c(-c3cn[nH]c3)c[nH]c12)c1cccc(CO)c1 ; NC[C@@H](C(=O)Nc1cccc2c(-c3cn[nH]c3)c[nH]c12)c1cccc(CO)c1]

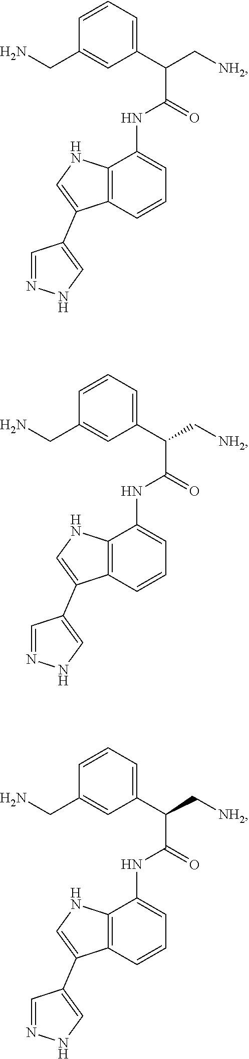 [SMILES: NCc1cccc(C(CN)C(=O)Nc2cccc3c(-c4cn[nH]c4)c[nH]c23)c1 ; NCc1cccc([C@H](CN)C(=O)Nc2cccc3c(-c4cn[nH]c4)c[nH]c23)c1 ; NCc1cccc([C@@H](CN)C(=O)Nc2cccc3c(-c4cn[nH]c4)c[nH]c23)c1]

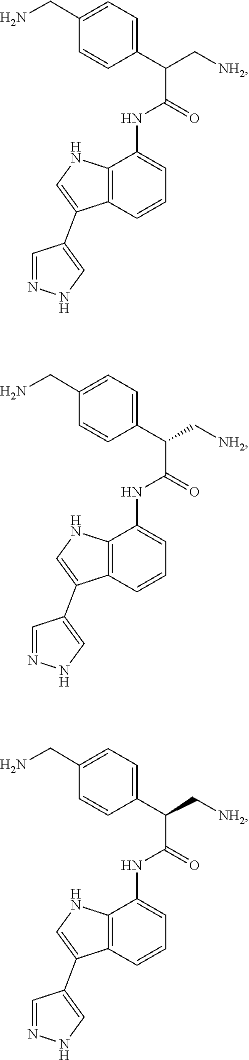 [SMILES: NCc1ccc(C(CN)C(=O)Nc2cccc3c(-c4cn[nH]c4)c[nH]c23)cc1 ; NCc1ccc([C@H](CN)C(=O)Nc2cccc3c(-c4cn[nH]c4)c[nH]c23)cc1 ; NCc1ccc([C@@H](CN)C(=O)Nc2cccc3c(-c4cn[nH]c4)c[nH]c23)cc1]

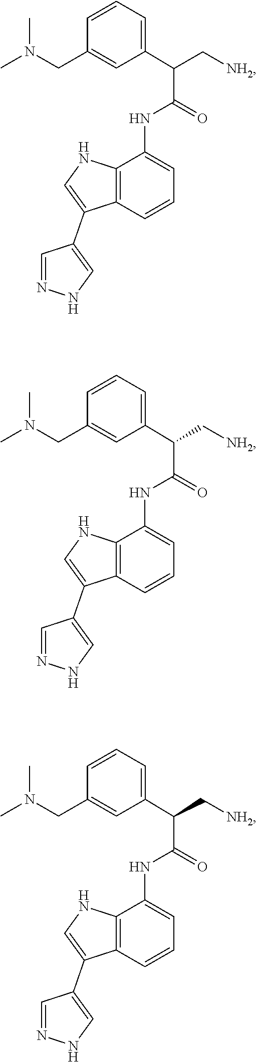 [SMILES: CN(C)Cc1cccc(C(CN)C(=O)Nc2cccc3c(-c4cn[nH]c4)c[nH]c23)c1 ; CN(C)Cc1cccc([C@H](CN)C(=O)Nc2cccc3c(-c4cn[nH]c4)c[nH]c23)c1 ; CN(C)Cc1cccc([C@@H](CN)C(=O)Nc2cccc3c(-c4cn[nH]c4)c[nH]c23)c1]

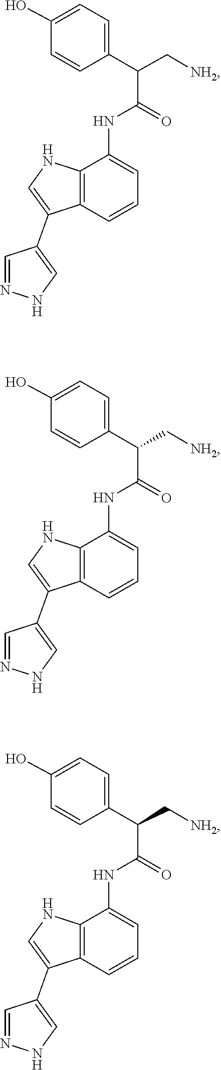 [SMILES: NCC(C(=O)Nc1cccc2c(-c3cn[nH]c3)c[nH]c12)c1ccc(O)cc1 ; NC[C@H](C(=O)Nc1cccc2c(-c3cn[nH]c3)c[nH]c12)c1ccc(O)cc1 ; NC[C@@H](C(=O)Nc1cccc2c(-c3cn[nH]c3)c[nH]c12)c1ccc(O)cc1]

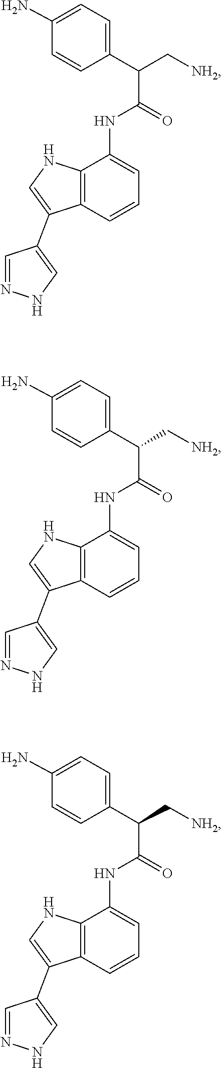 [SMILES: NCC(C(=O)Nc1cccc2c(-c3cn[nH]c3)c[nH]c12)c1ccc(N)cc1 ; NC[C@H](C(=O)Nc1cccc2c(-c3cn[nH]c3)c[nH]c12)c1ccc(N)cc1 ; NC[C@@H](C(=O)Nc1cccc2c(-c3cn[nH]c3)c[nH]c12)c1ccc(N)cc1]

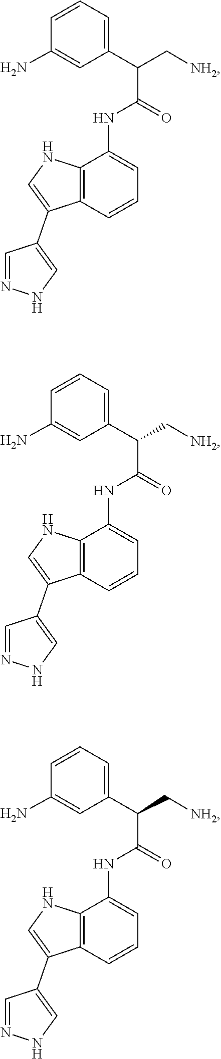 [SMILES: NCC(C(=O)Nc1cccc2c(-c3cn[nH]c3)c[nH]c12)c1cccc(N)c1 ; NC[C@H](C(=O)Nc1cccc2c(-c3cn[nH]c3)c[nH]c12)c1cccc(N)c1 ; NC[C@@H](C(=O)Nc1cccc2c(-c3cn[nH]c3)c[nH]c12)c1cccc(N)c1]

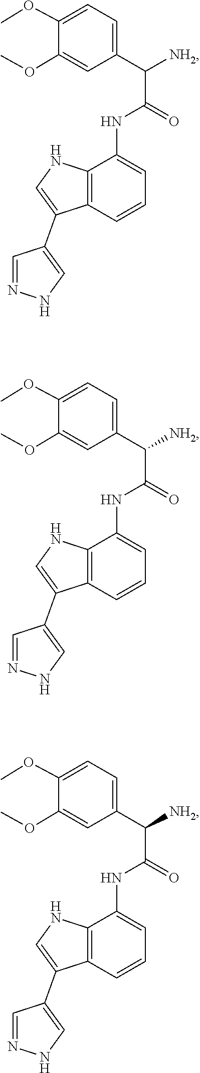 [SMILES: COc1ccc(C(N)C(=O)Nc2cccc3c(-c4cn[nH]c4)c[nH]c23)cc1OC ; COc1ccc([C@H](N)C(=O)Nc2cccc3c(-c4cn[nH]c4)c[nH]c23)cc1OC ; COc1ccc([C@@H](N)C(=O)Nc2cccc3c(-c4cn[nH]c4)c[nH]c23)cc1OC]

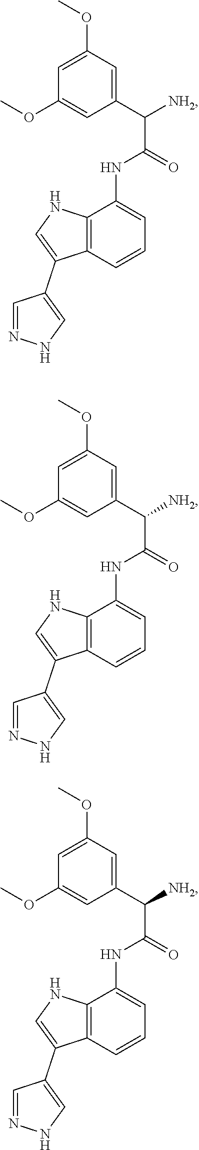

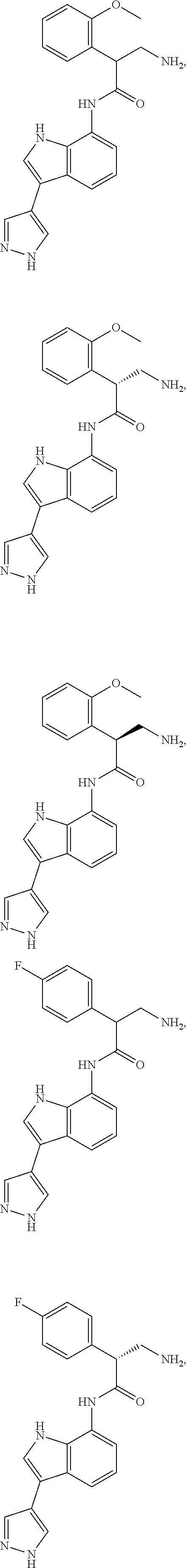

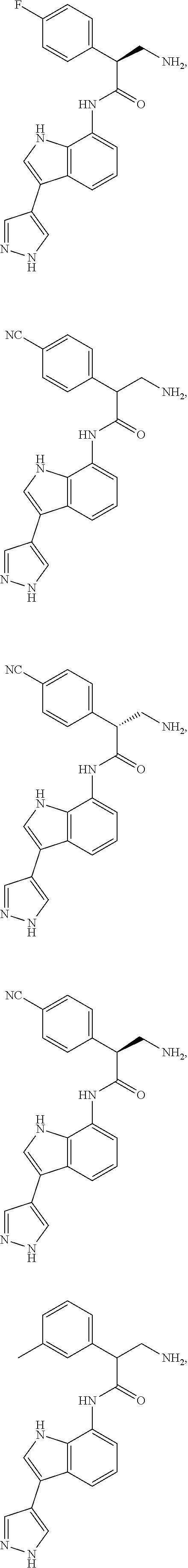 [SMILES: NC[C@@H](C(=O)Nc1cccc2c(-c3cn[nH]c3)c[nH]c12)c1ccc(F)cc1 ; N#Cc1ccc(C(CN)C(=O)Nc2cccc3c(-c4cn[nH]c4)c[nH]c23)cc1 ; N#Cc1ccc([C@H](CN)C(=O)Nc2cccc3c(-c4cn[nH]c4)c[nH]c23)cc1 ; N#Cc1ccc([C@@H](CN)C(=O)Nc2cccc3c(-c4cn[nH]c4)c[nH]c23)cc1 ; Cc1cccc(C(CN)C(=O)Nc2cccc3c(-c4cn[nH]c4)c[nH]c23)c1]

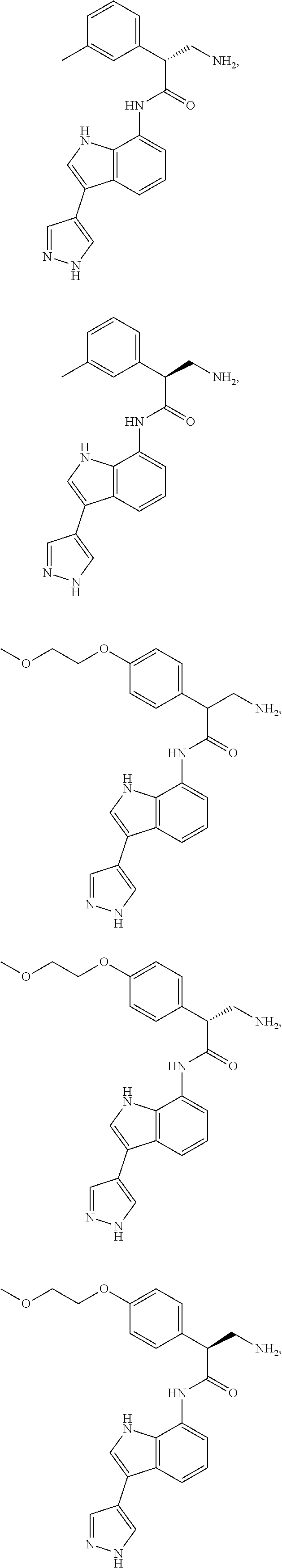 [SMILES: Cc1cccc([C@H](CN)C(=O)Nc2cccc3c(-c4cn[nH]c4)c[nH]c23)c1 ; Cc1cccc([C@@H](CN)C(=O)Nc2cccc3c(-c4cn[nH]c4)c[nH]c23)c1 ; COCCOc1ccc(C(CN)C(=O)Nc2cccc3c(-c4cn[nH]c4)c[nH]c23)cc1 ; COCCOc1ccc([C@H](CN)C(=O)Nc2cccc3c(-c4cn[nH]c4)c[nH]c23)cc1 ; COCCOc1ccc([C@@H](CN)C(=O)Nc2cccc3c(-c4cn[nH]c4)c[nH]c23)cc1]

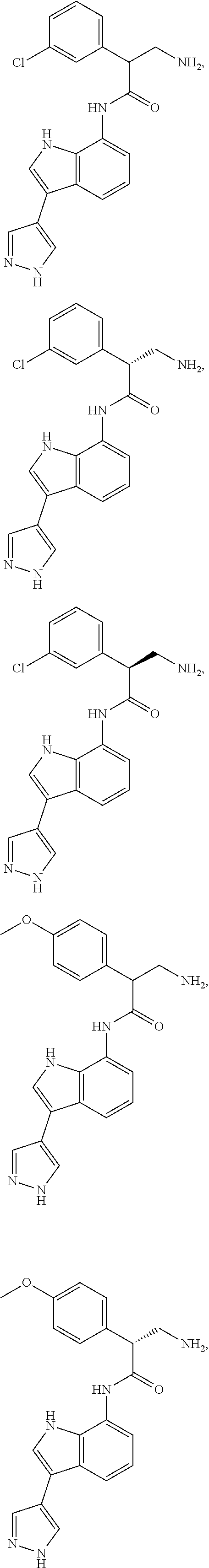 [SMILES: NCC(C(=O)Nc1cccc2c(-c3cn[nH]c3)c[nH]c12)c1cccc(Cl)c1 ; NC[C@H](C(=O)Nc1cccc2c(-c3cn[nH]c3)c[nH]c12)c1cccc(Cl)c1 ; NC[C@@H](C(=O)Nc1cccc2c(-c3cn[nH]c3)c[nH]c12)c1cccc(Cl)c1 ; COc1ccc(C(CN)C(=O)Nc2cccc3c(-c4cn[nH]c4)c[nH]c23)cc1 ; COc1ccc([C@H](CN)C(=O)Nc2cccc3c(-c4cn[nH]c4)c[nH]c23)cc1]

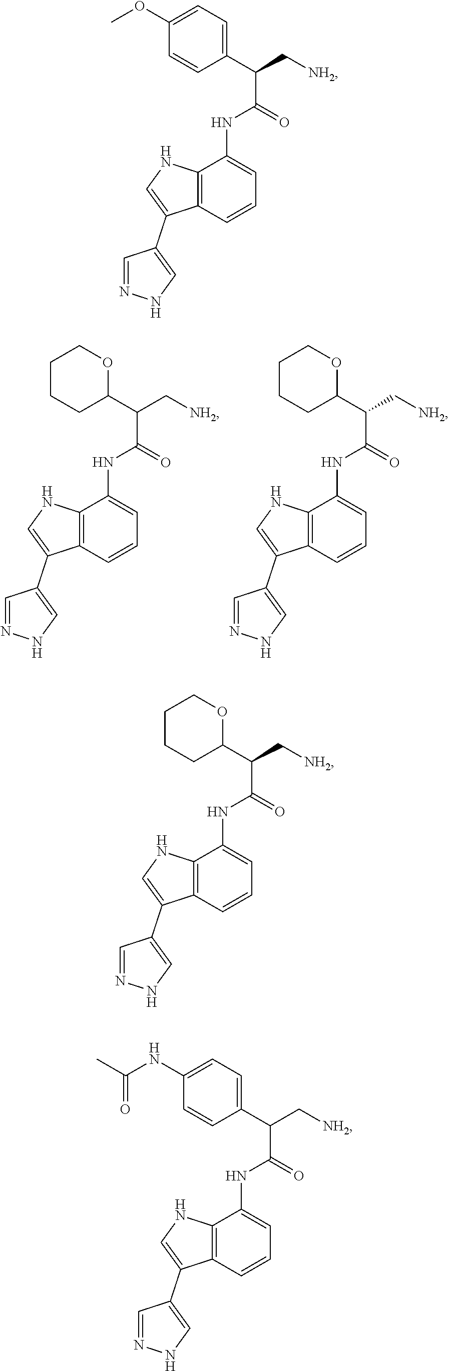 [SMILES: COc1ccc([C@@H](CN)C(=O)Nc2cccc3c(-c4cn[nH]c4)c[nH]c23)cc1 ; NCC(C(=O)Nc1cccc2c(-c3cn[nH]c3)c[nH]c12)C1CCCCO1 ; NC[C@H](C(=O)Nc1cccc2c(-c3cn[nH]c3)c[nH]c12)C1CCCCO1 ; NC[C@@H](C(=O)Nc1cccc2c(-c3cn[nH]c3)c[nH]c12)C1CCCCO1 ; CC(=O)Nc1ccc(C(CN)C(=O)Nc2cccc3c(-c4cn[nH]c4)c[nH]c23)cc1]

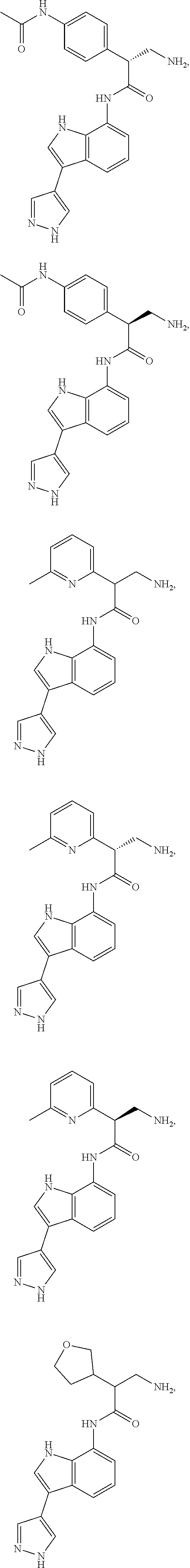 [SMILES: CC(=O)Nc1ccc([C@H](CN)C(=O)Nc2cccc3c(-c4cn[nH]c4)c[nH]c23)cc1 ; CC(=O)Nc1ccc([C@@H](CN)C(=O)Nc2cccc3c(-c4cn[nH]c4)c[nH]c23)cc1 ; Cc1cccc(C(CN)C(=O)Nc2cccc3c(-c4cn[nH]c4)c[nH]c23)n1 ; Cc1cccc([C@H](CN)C(=O)Nc2cccc3c(-c4cn[nH]c4)c[nH]c23)n1 ; Cc1cccc([C@@H](CN)C(=O)Nc2cccc3c(-c4cn[nH]c4)c[nH]c23)n1 ; NCC(C(=O)Nc1cccc2c(-c3cn[nH]c3)c[nH]c12)C1CCOC1]

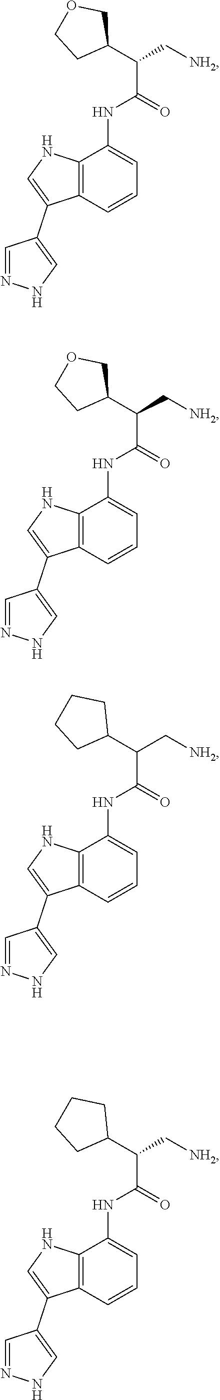 [SMILES: NC[C@H](C(=O)Nc1cccc2c(-c3cn[nH]c3)c[nH]c12)[C@H]1CCOC1 ; NC[C@@H](C(=O)Nc1cccc2c(-c3cn[nH]c3)c[nH]c12)[C@H]1CCOC1 ; NCC(C(=O)Nc1cccc2c(-c3cn[nH]c3)c[nH]c12)C1CCCC1 ; NC[C@H](C(=O)Nc1cccc2c(-c3cn[nH]c3)c[nH]c12)C1CCCC1]

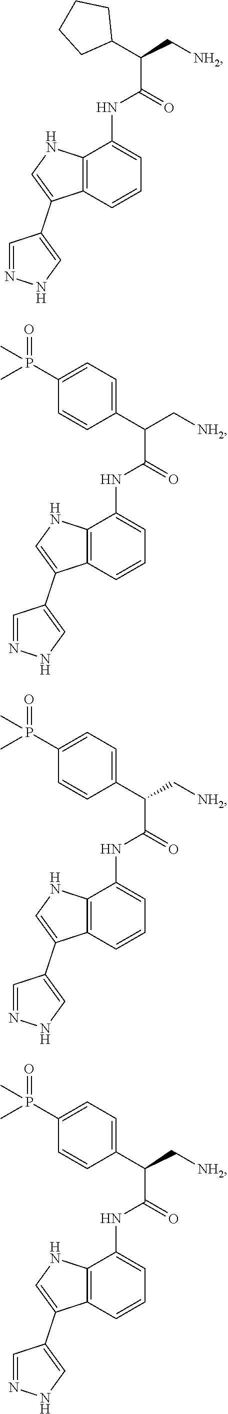

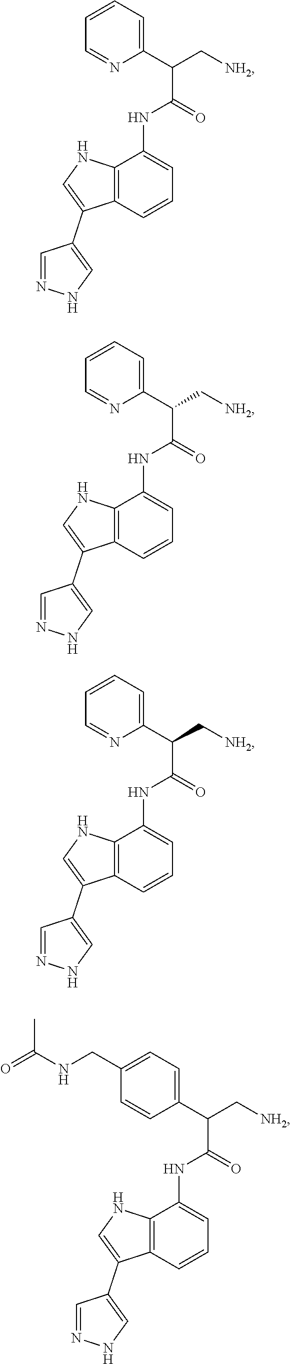 [SMILES: NCC(C(=O)Nc1cccc2c(-c3cn[nH]c3)c[nH]c12)c1ccccn1 ; NC[C@H](C(=O)Nc1cccc2c(-c3cn[nH]c3)c[nH]c12)c1ccccn1 ; NC[C@@H](C(=O)Nc1cccc2c(-c3cn[nH]c3)c[nH]c12)c1ccccn1 ; CC(=O)NCc1ccc(C(CN)C(=O)Nc2cccc3c(-c4cn[nH]c4)c[nH]c23)cc1]

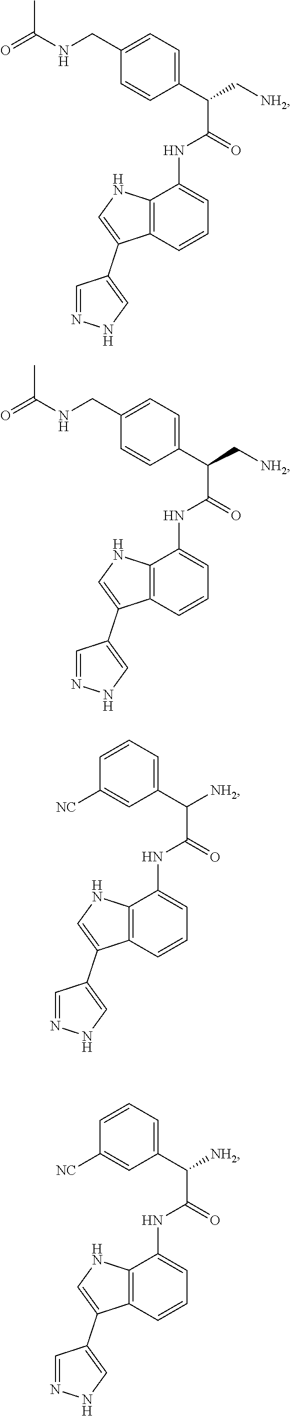 [SMILES: CC(=O)NCc1ccc([C@H](CN)C(=O)Nc2cccc3c(-c4cn[nH]c4)c[nH]c23)cc1 ; CC(=O)NCc1ccc([C@@H](CN)C(=O)Nc2cccc3c(-c4cn[nH]c4)c[nH]c23)cc1 ; N#Cc1cccc(C(N)C(=O)Nc2cccc3c(-c4cn[nH]c4)c[nH]c23)c1 ; N#Cc1cccc([C@H](N)C(=O)Nc2cccc3c(-c4cn[nH]c4)c[nH]c23)c1]

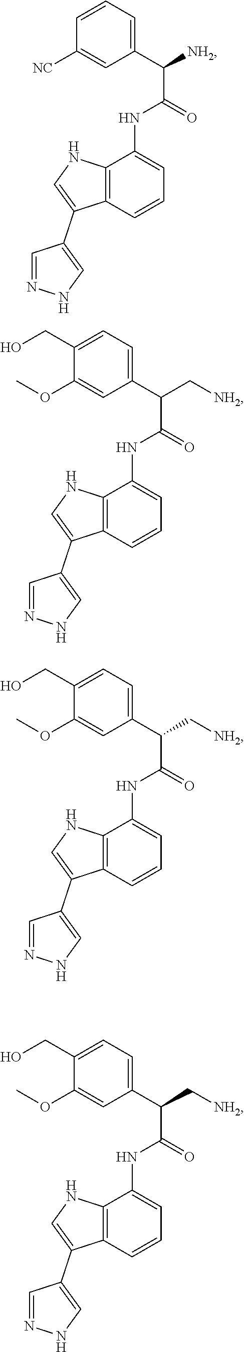

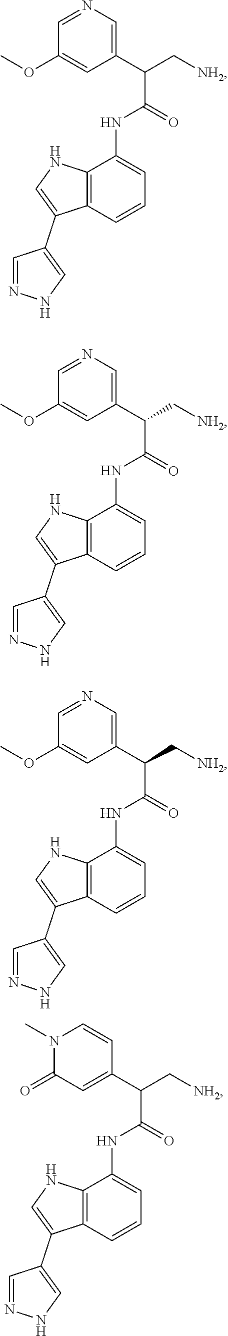

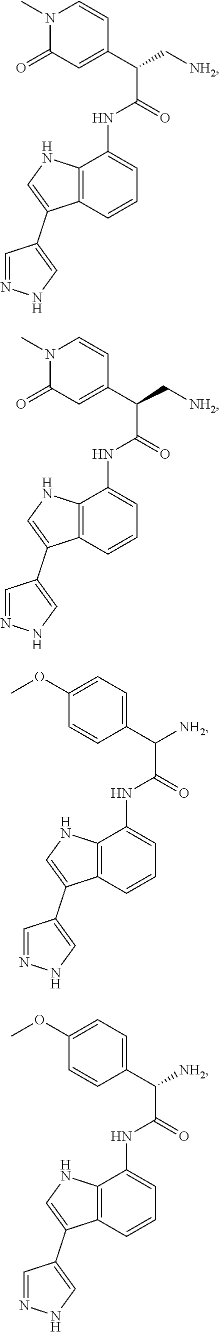

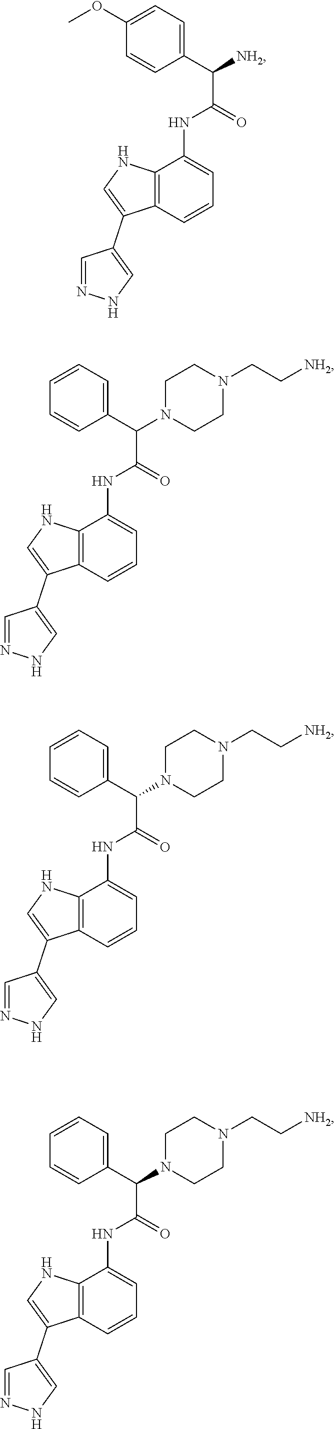 [SMILES: COc1ccc([C@@H](N)C(=O)Nc2cccc3c(-c4cn[nH]c4)c[nH]c23)cc1 ; NCCN1CCN(C(C(=O)Nc2cccc3c(-c4cn[nH]c4)c[nH]c23)c2ccccc2)CC1 ; NCCN1CCN([C@H](C(=O)Nc2cccc3c(-c4cn[nH]c4)c[nH]c23)c2ccccc2)CC1 ; NCCN1CCN([C@@H](C(=O)Nc2cccc3c(-c4cn[nH]c4)c[nH]c23)c2ccccc2)CC1]

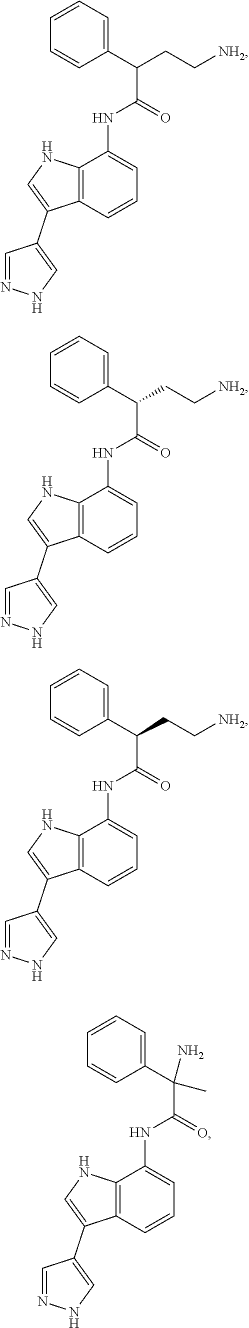 [SMILES: NCCC(C(=O)Nc1cccc2c(-c3cn[nH]c3)c[nH]c12)c1ccccc1 ; NCC[C@H](C(=O)Nc1cccc2c(-c3cn[nH]c3)c[nH]c12)c1ccccc1 ; NCC[C@@H](C(=O)Nc1cccc2c(-c3cn[nH]c3)c[nH]c12)c1ccccc1 ; CC(N)(C(=O)Nc1cccc2c(-c3cn[nH]c3)c[nH]c12)c1ccccc1]

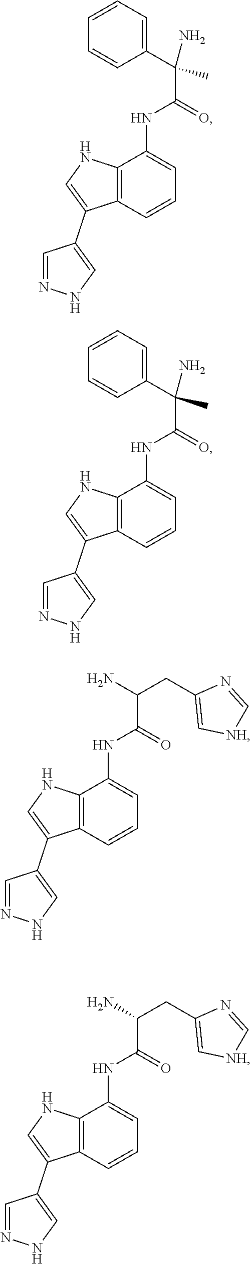 [SMILES: C[C@](N)(C(=O)Nc1cccc2c(-c3cn[nH]c3)c[nH]c12)c1ccccc1 ; C[C@@](N)(C(=O)Nc1cccc2c(-c3cn[nH]c3)c[nH]c12)c1ccccc1 ; NC(Cc1c[nH]cn1)C(=O)Nc1cccc2c(-c3cn[nH]c3)c[nH]c12 ; N[C@H](Cc1c[nH]cn1)C(=O)Nc1cccc2c(-c3cn[nH]c3)c[nH]c12]

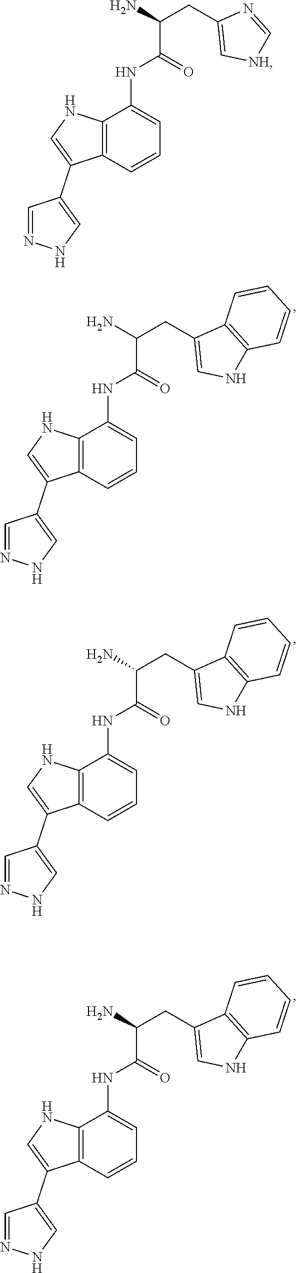 [SMILES: N[C@@H](Cc1c[nH]cn1)C(=O)Nc1cccc2c(-c3cn[nH]c3)c[nH]c12 ; NC(Cc1c[nH]c2ccccc12)C(=O)Nc1cccc2c(-c3cn[nH]c3)c[nH]c12 ; N[C@H](Cc1c[nH]c2ccccc12)C(=O)Nc1cccc2c(-c3cn[nH]c3)c[nH]c12 ; N[C@@H](Cc1c[nH]c2ccccc12)C(=O)Nc1cccc2c(-c3cn[nH]c3)c[nH]c12]

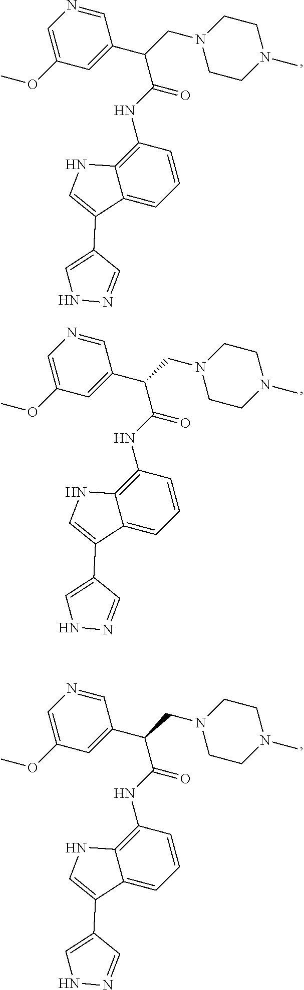

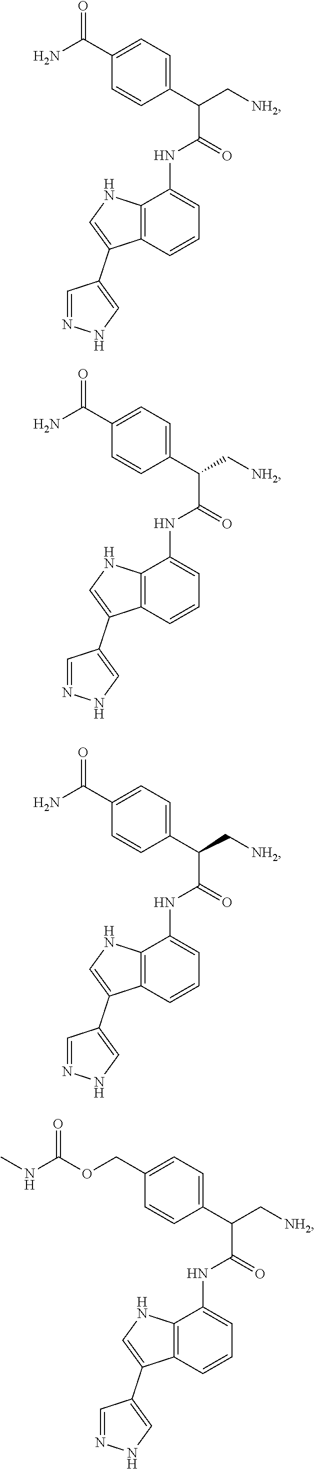

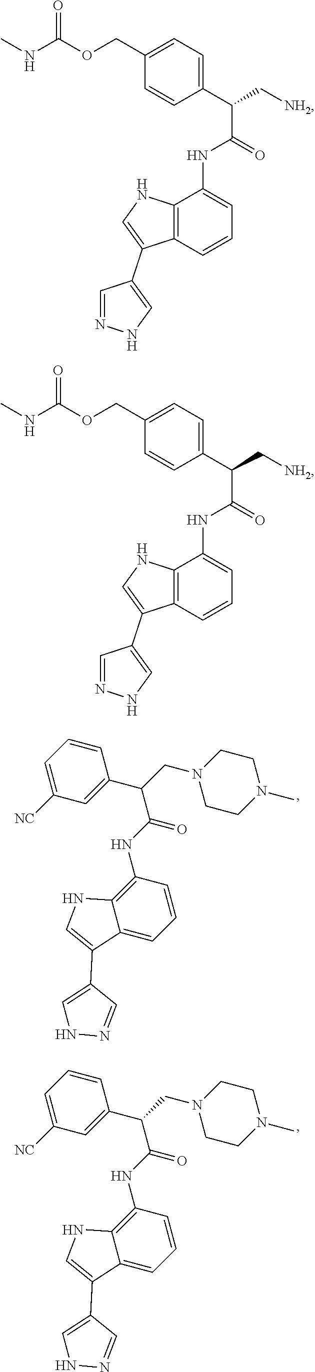

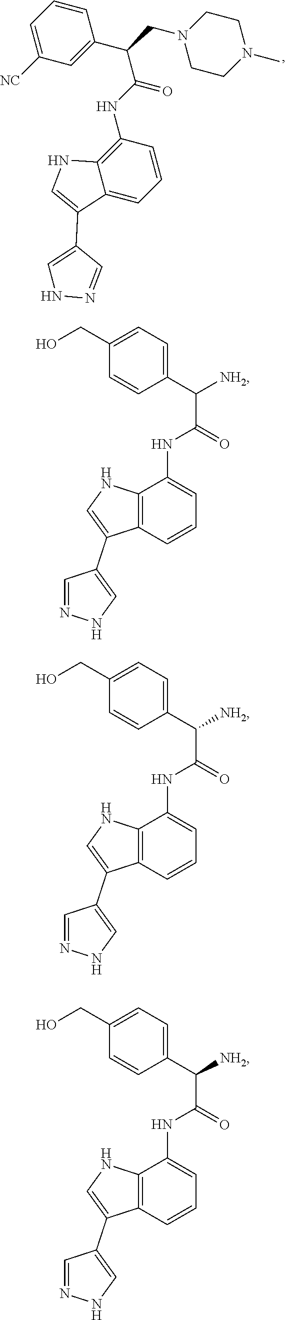

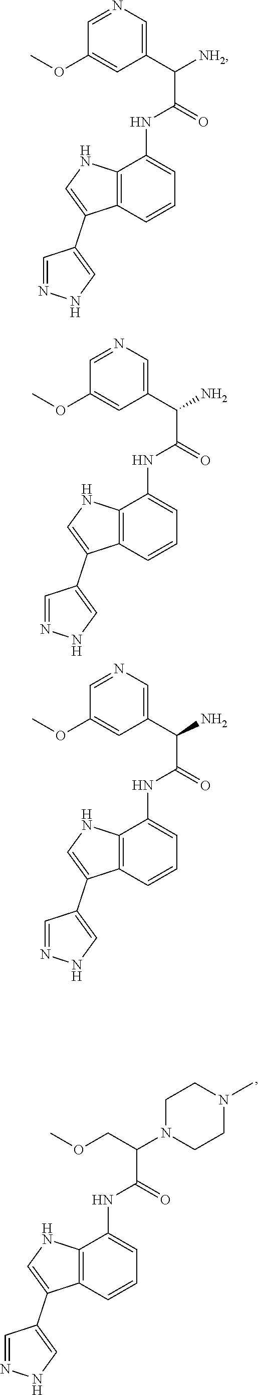 [SMILES: COc1cncc(C(N)C(=O)Nc2cccc3c(-c4cn[nH]c4)c[nH]c23)c1 ; COc1cncc([C@H](N)C(=O)Nc2cccc3c(-c4cn[nH]c4)c[nH]c23)c1 ; COc1cncc([C@@H](N)C(=O)Nc2cccc3c(-c4cn[nH]c4)c[nH]c23)c1 ; COCC(C(=O)Nc1cccc2c(-c3cn[nH]c3)c[nH]c12)N1CCN(C)CC1]

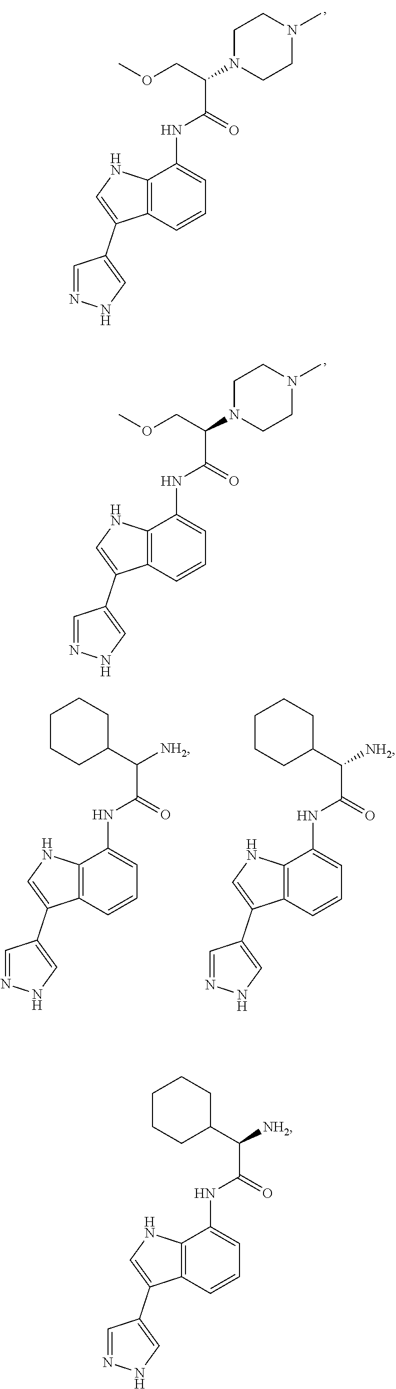

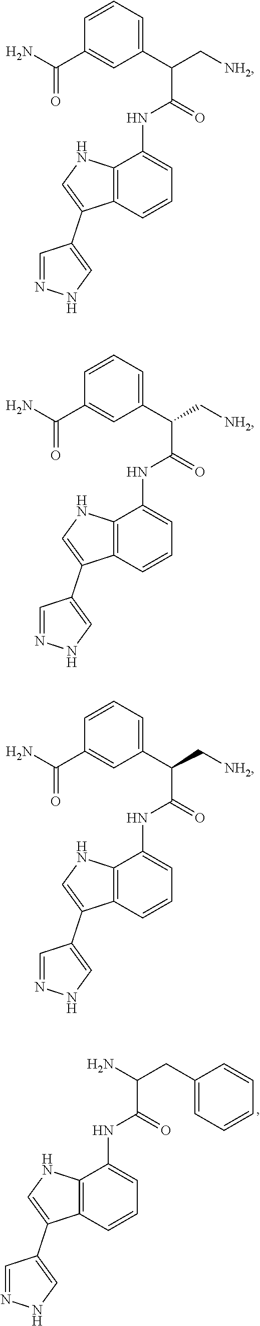 [SMILES: NCC(C(=O)Nc1cccc2c(-c3cn[nH]c3)c[nH]c12)c1cccc(C(N)=O)c1 ; NC[C@H](C(=O)Nc1cccc2c(-c3cn[nH]c3)c[nH]c12)c1cccc(C(N)=O)c1 ; NC[C@@H](C(=O)Nc1cccc2c(-c3cn[nH]c3)c[nH]c12)c1cccc(C(N)=O)c1 ; NC(Cc1ccccc1)C(=O)Nc1cccc2c(-c3cn[nH]c3)c[nH]c12]

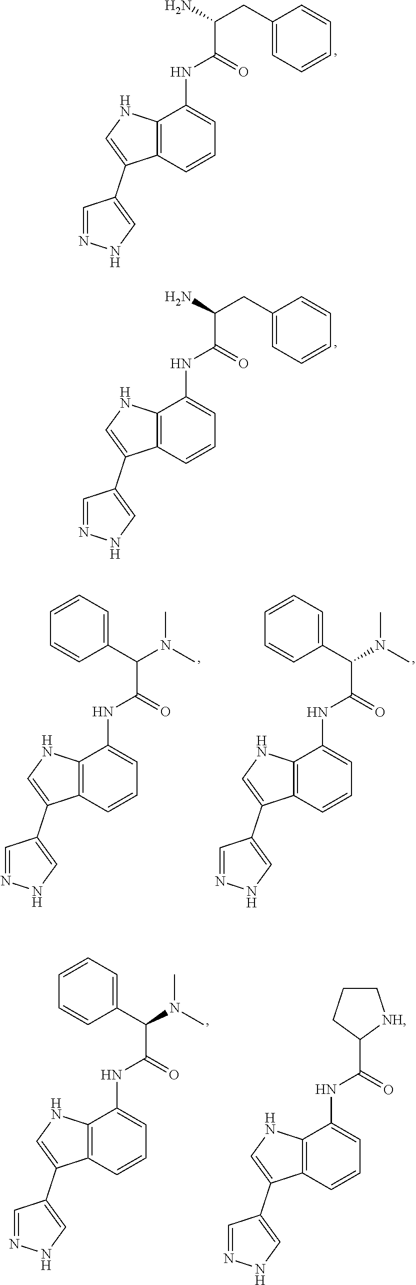 [SMILES: N[C@H](Cc1ccccc1)C(=O)Nc1cccc2c(-c3cn[nH]c3)c[nH]c12 ; N[C@@H](Cc1ccccc1)C(=O)Nc1cccc2c(-c3cn[nH]c3)c[nH]c12 ; CN(C)C(C(=O)Nc1cccc2c(-c3cn[nH]c3)c[nH]c12)c1ccccc1 ; CN(C)[C@H](C(=O)Nc1cccc2c(-c3cn[nH]c3)c[nH]c12)c1ccccc1 ; CN(C)[C@@H](C(=O)Nc1cccc2c(-c3cn[nH]c3)c[nH]c12)c1ccccc1 ; O=C(Nc1cccc2c(-c3cn[nH]c3)c[nH]c12)C1CCCN1]

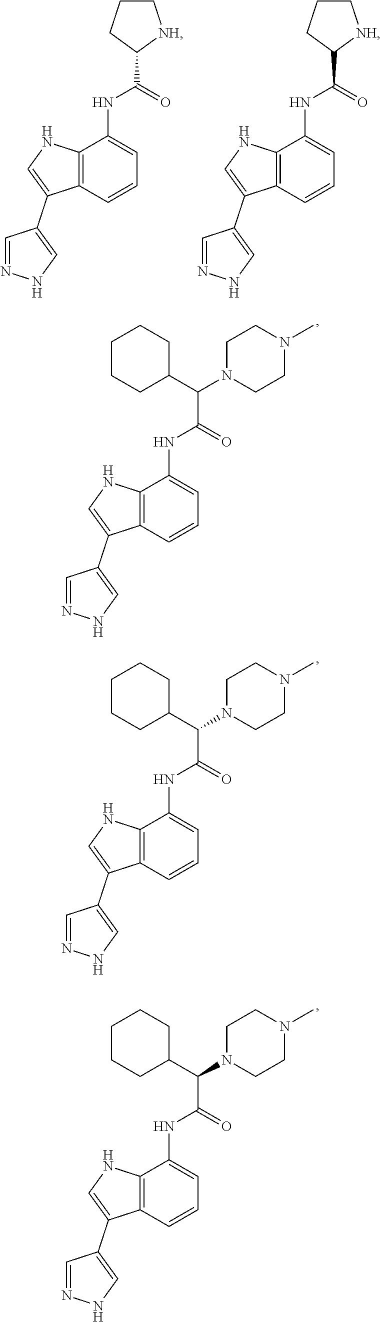

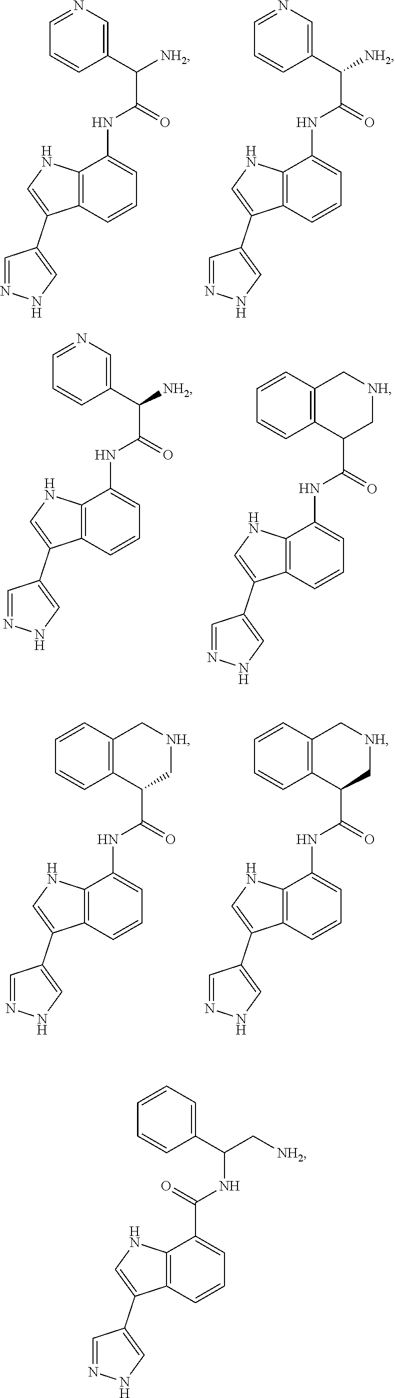

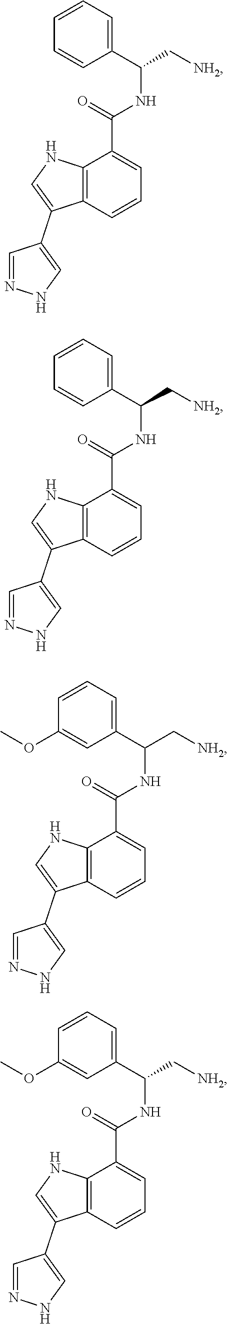 [SMILES: NC[C@H](NC(=O)c1cccc2c(-c3cn[nH]c3)c[nH]c12)c1ccccc1 ; NC[C@@H](NC(=O)c1cccc2c(-c3cn[nH]c3)c[nH]c12)c1ccccc1 ; COc1cccc(C(CN)NC(=O)c2cccc3c(-c4cn[nH]c4)c[nH]c23)c1 ; COc1cccc([C@H](CN)NC(=O)c2cccc3c(-c4cn[nH]c4)c[nH]c23)c1]

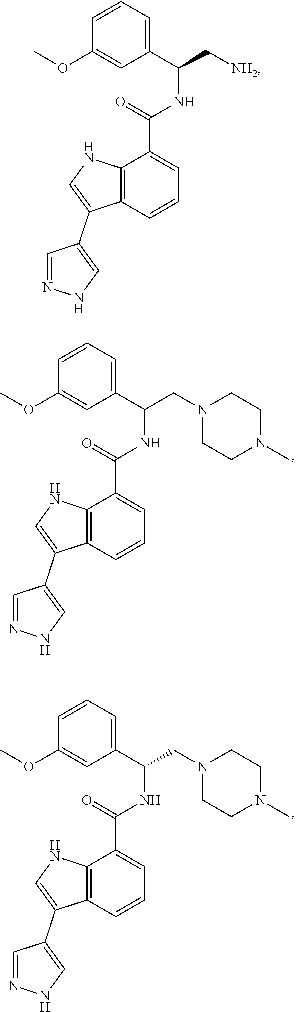

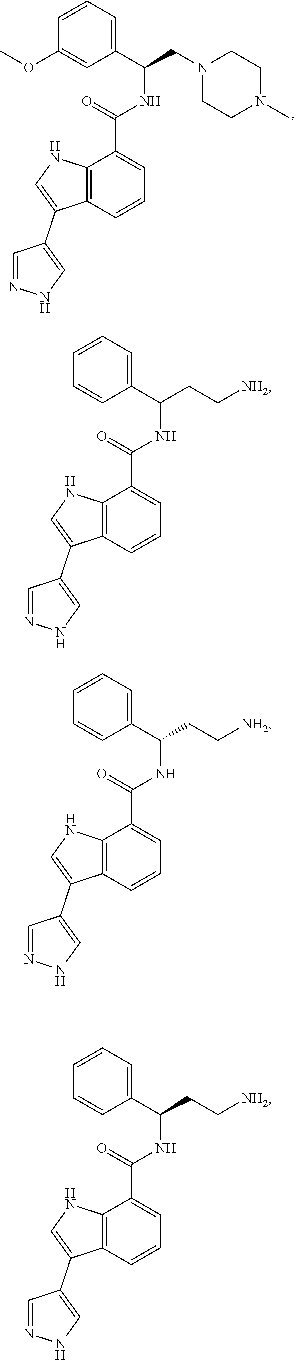

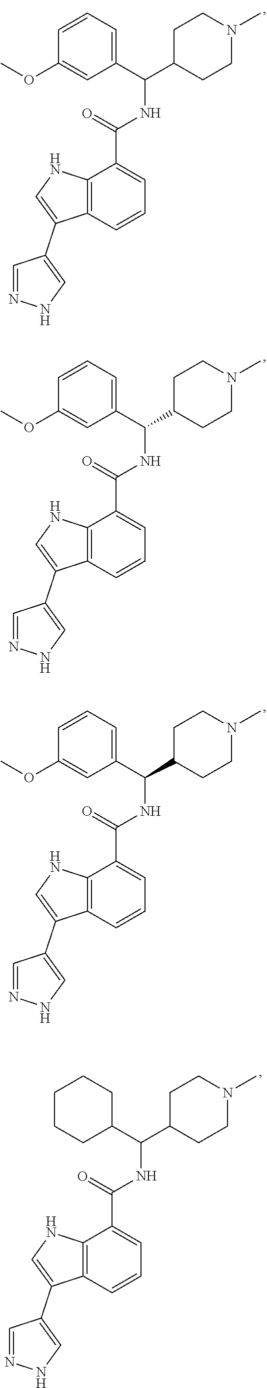

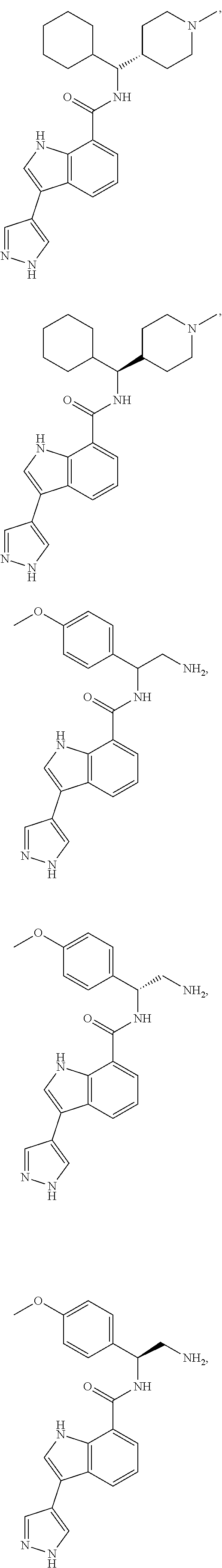

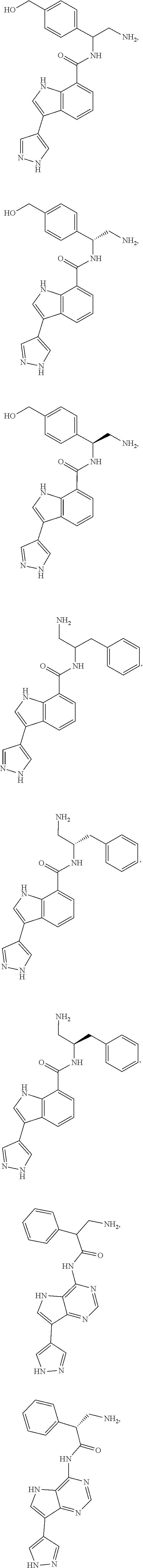

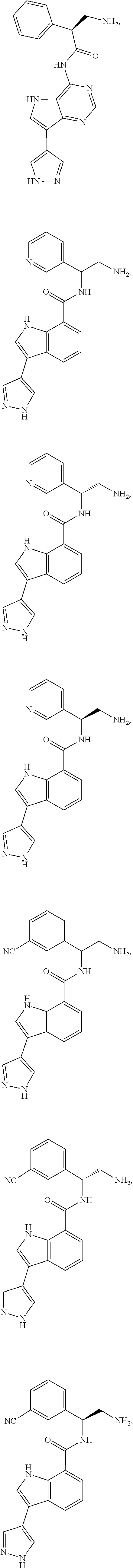

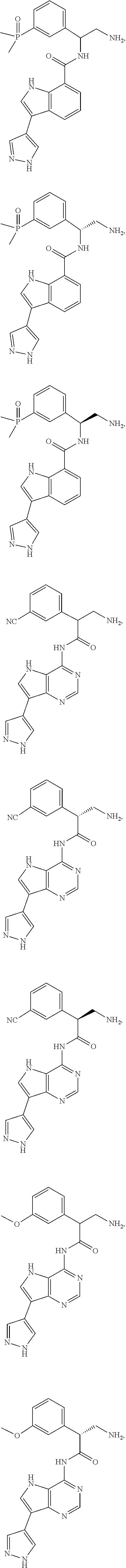

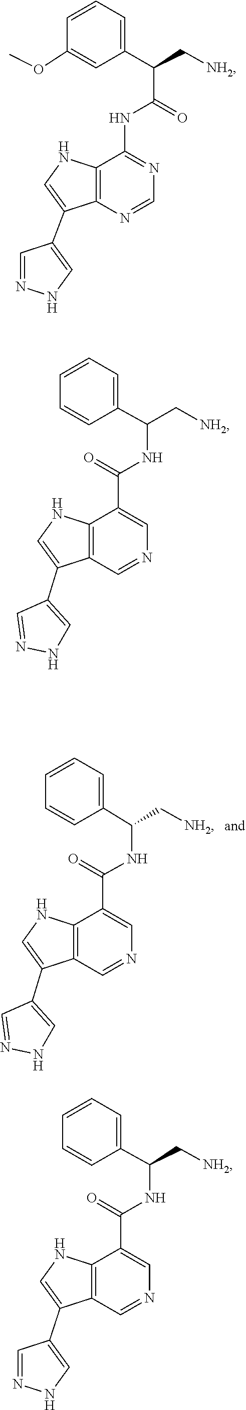 [SMILES: COc1cccc([C@@H](CN)C(=O)Nc2ncnc3c(-c4cn[nH]c4)c[nH]c23)c1 ; NCC(NC(=O)c1cncc2c(-c3cn[nH]c3)c[nH]c12)c1ccccc1 ; NC[C@H](NC(=O)c1cncc2c(-c3cn[nH]c3)c[nH]c12)c1ccccc1 ; NC[C@@H](NC(=O)c1cncc2c(-c3cn[nH]c3)c[nH]c12)c1ccccc1]

46. A pharmaceutical composition comprising the compound or a pharmaceutically acceptable salt thereof of claim 1, and a pharmaceutically acceptable carrier.

47. The pharmaceutical composition of claim 46, which is formulated as eye-drops.

48. A method of inhibiting ROCK activity or treating a ROCK-related disorder in a subject in need thereof, comprising administering an effective amount of the compound or a pharmaceutically acceptable salt thereof of claim 1.

49. (canceled)

50. The method according to claim 4948, wherein the ROCK-related disorder is eye disease including glaucoma and retinal diseases such as Wet AMD, Dry AMD and DME, bone disorder including osteoporosis, vascular disease including cerebral vasospasm, coronary vasospasm, hypertension, pulmonary hypertension, sudden death syndrome, angina, myocardial infarction, restenosis, stroke, hypertensive vascular disease, heart failure, cardiac allograft vasculopathy, vein graft disease, pulmonary disease including chronic obstructive pulmonary disease (COPD) and asthma, neurological disorder including spinal cord injury, Alzheimer's disease, multiple sclerosis, depression, attention deficit-hyperactivity disorder and neuropathic pain, neovascular disorders and cancer, obesity, and erectile dysfunction.

51. The method according to claim 49, wherein the ROCK-related disorder is glaucoma.

52. The method according to claim 48, wherein the compound is administered simultaneously, separately or sequentially with one or more additional therapeutic agents selected from beta blockers, alpha-agonists, carbonic anhydrase inhibitors, prostaglandin-like compounds, miotic or cholinergic agents, or epinephrine compounds.

53. (canceled)
